SEQUENCE LISTING (1325 sequences)

SEQ ID NO: 1 - protein, 21 aa, Borrelia burgdorferi
KASLEVASSSQNLSSSALQQA

SEQ ID NO: 2 - protein, 12 aa, Borrelia burgdorferi
NVFDFQFLFAMK

SEQ ID NO: 3 - protein, 16 aa, Borrelia burgdorferi
HSESVITPVVIANSDE

SEQ ID NO: 4 - protein, 14 aa, Borrelia burgdorferi
NAEEYFDGKANESE

SEQ ID NO: 5 - protein, 273 aa, unidentified
MKKYLLGIGLILALIACKQNVSSLDEKNSVSVDLPGEIKVLVSKEKDKDGKYSLMATVDKLELKGTSDKNNGSGVLEGVKADKSKVKLTVSDDLSKTTLEVLKEDGKTLVSRKVTSKDKSSTEEKFNEKGELAEKTMTRADETRLEYTEIKSDGSGKAKEVLKGYALEGTLTAEKTTLVVKEGTVTLSKNISKSGEVTAELNDTDSAAATKKTGAWNSGTSTLTITANSKKTKDLVFTKENTITVQKYDTAGIKLEGSAVEIKTLDELKNALK

SEQ ID NO: 6 - protein, 199 aa, unidentified
ILMTLFLFISCNNSGKDGNTSANSADESVKGPNLTEISKKITDSNAVLLAVKEVEALLSSIDELAKAIGKKIKNDGSLGDEANHNESLLAGAYTISTLITQKLSKLNGSEGLKEKIAAAKKCSEEFSTKLKDNHAQLGIQGVTDENAKKAILKANAAGKDKGVEELEKLSGSLESLSKAAKEMLANSVKELTSPVVAES

SEQ ID NO: 7 - protein, 40 aa, Homo sapiens
FDVDSDRHLVNPANGMRIQGWMARDLEGEKVINTASDIED

SEQ ID NO: 8 - protein, 37 aa, Homo sapiens
VFQFSVDIAKNFDNIEDGIKEYESKINKFEYRSGWEA

SEQ ID NO: 9 - protein, 42 aa, Homo sapiens
AIDEEKKCIAQANAAFNSNGKYLEDLVSVRISGDYTTTSPTN

SEQ ID NO: 10 - protein, 33 aa, Homo sapiens
KNTISLSKGIIACSPYILIVTFIVLVSPLFNKI

SEQ ID NO: 11 - protein, 18 aa, Homo sapiens
KASISTLKVGFESLQRIL

SEQ ID NO: 12 - protein, 20 aa, Homo sapiens
KNIKYEDVIQKNDNFANISL

SEQ ID NO: 13 - protein, 20 aa, Homo sapiens
IVFAEEKDNTVRALQDSFRE

SEQ ID NO: 14 - protein, 18 aa, Homo sapiens
ALNFLGPAIAVFISTLIF

SEQ ID NO: 15 - protein, 42 aa, Homo sapiens
LEYLKEHSESVITPVVIANSDEVSSIEYSFGRLENDSEYILS

SEQ ID NO: 16 - protein, 19 aa, Homo sapiens
YVTWAKSGDLRDIKDKNNN

SEQ ID NO: 17 - protein, 20 aa, Homo sapiens
NGVIIKAGGGPPTAINIYAL

SEQ ID NO: 18 - protein, 15 aa, Homo sapiens
ATTLTEAGYVGEDVE

SEQ ID NO: 19 - protein, 9 aa, Homo sapiens
KIESDLILY

SEQ ID NO: 20 - protein, 16 aa, Homo sapiens
TFKQVVQEALKGGIDG

SEQ ID NO: 21 - protein, 10 aa, Homo sapiens
ESIGPIFNQL

SEQ ID NO: 22 - protein, 10 aa, Homo sapiens
SEIIRNSGQI

SEQ ID NO: 23 - protein, 12 aa, Mus sp.
NVFDFQFLFAMK

SEQ ID NO: 24 - protein, 16 aa, Mus sp.
HSESVITPVVIANSDE

SEQ ID NO: 25 - protein, 38 aa, Mus sp.
KPMILTRTSSYIGVLIDDLVTKGTKEPYRMFTSRAEHR

SEQ ID NO: 26 - protein, 21 aa, Mus sp.
KASLEVASSSQNLSSSALQQA

SEQ ID NO: 27 - protein, 15 aa, Borrelia burgdorferi
EDYYKQLTRAQVRRA

SEQ ID NO: 28 - protein, 15 aa, Borrelia burgdorferi
DYYKQLTRAQVRRAA

SEQ ID NO: 29 - protein, 15 aa, Borrelia burgdorferi
YYKQLTRAQVRRAAF

SEQ ID NO: 30 - protein, 15 aa, Borrelia burgdorferi
YKQLTRAQVRRAAFS

SEQ ID NO: 31 - protein, 15 aa, Borrelia burgdorferi
LDTLHVIINGAASNL

SEQ ID NO: 32 - protein, 15 aa, Borrelia burgdorferi
LHVIINGAASNLALE

SEQ ID NO: 33 - protein, 15 aa, Borrelia burgdorferi
HVIINGAASNLALET

SEQ ID NO: 34 - protein, 15 aa, Borrelia burgdorferi
ISEFAMSENRGKDFS

SEQ ID NO: 35 - protein, 15 aa, Borrelia burgdorferi
ESELIDLRKNPKFVI

SEQ ID NO: 36 - protein, 15 aa, Borrelia burgdorferi
KNPKFVIDSVKVSKK

SEQ ID NO: 37 - protein, 15 aa, Borrelia burgdorferi
NPKFVIDSVKVSKKY

SEQ ID NO: 38 - protein, 15 aa, Borrelia burgdorferi
PKFVIDSVKVSKKYR

SEQ ID NO: 39 - protein, 15 aa, Borrelia burgdorferi
KFVIDSVKVSKKYRQ

SEQ ID NO: 40 - protein, 15 aa, Borrelia burgdorferi
FVIDSVKVSKKYRQY

SEQ ID NO: 41 - protein, 15 aa, Borrelia burgdorferi
RQYLYNFMANLKNDT

SEQ ID NO: 42 - protein, 15 aa, Borrelia burgdorferi
FEGRVIVSTRHENNM

SEQ ID NO: 43 - protein, 15 aa, Borrelia burgdorferi
EGRVIVSTRHENNMD

SEQ ID NO: 44 - protein, 15 aa, Borrelia burgdorferi
NMDFGHSEANTNYFK

SEQ ID NO: 45 - protein, 15 aa, Borrelia burgdorferi
NTNYFKKAVEDYRQN

SEQ ID NO: 46 - protein, 15 aa, Borrelia burgdorferi
TNYFKKAVEDYRQNQ

SEQ ID NO: 47 - protein, 15 aa, Borrelia burgdorferi
SNLSEGISAEVAIRS

SEQ ID NO: 48 - protein, 15 aa, Borrelia burgdorferi
NLSEGISAEVAIRSK

SEQ ID NO: 49 - protein, 15 aa, Borrelia burgdorferi
LSEGISAEVAIRSKQ

SEQ ID NO: 50 - protein, 15 aa, Borrelia burgdorferi
SEGISAEVAIRSKQS

SEQ ID NO: 51 - protein, 15 aa, Borrelia burgdorferi
ISAEVAIRSKQSEKK

SEQ ID NO: 52 - protein, 15 aa, Borrelia burgdorferi
SAEVAIRSKQSEKKA

SEQ ID NO: 53 - protein, 15 aa, Borrelia burgdorferi
AEVAIRSKQSEKKAF

SEQ ID NO: 54 - protein, 15 aa, Borrelia burgdorferi
SEKKAFAIIVPVYSP

SEQ ID NO: 55 - protein, 15 aa, Borrelia burgdorferi
EKKAFAIIVPVYSPE

SEQ ID NO: 56 - protein, 15 aa, Borrelia burgdorferi
KKAFAIIVPVYSPED

SEQ ID NO: 57 - protein, 15 aa, Borrelia burgdorferi
RGNFIYVDPNNIAVN

SEQ ID NO: 58 - protein, 15 aa, Borrelia burgdorferi
GNFIYVDPNNIAVNP

SEQ ID NO: 59 - protein, 15 aa, Borrelia burgdorferi
SSKFLNVLKDVFSKP

SEQ ID NO: 60 - protein, 15 aa, Borrelia burgdorferi
VSVYTIDRILLSEMG

SEQ ID NO: 61 - protein, 15 aa, Borrelia burgdorferi
EDCYYAMLPISSKLG

SEQ ID NO: 62 - protein, 15 aa, Borrelia burgdorferi
EKSGVLIARLPYKDI

SEQ ID NO: 63 - protein, 15 aa, Borrelia burgdorferi
KSGVLIARLPYKDIY

SEQ ID NO: 64 - protein, 15 aa, Borrelia burgdorferi
YKDIYGVISSLRFQY

SEQ ID NO: 65 - protein, 15 aa, Borrelia burgdorferi
IISFRLNAIRVLVQD

SEQ ID NO: 66 - protein, 15 aa, Borrelia burgdorferi
LNAIRVLVQDMVKGN

SEQ ID NO: 67 - protein, 15 aa, Borrelia burgdorferi
MLSLQVVKMKKAISV

SEQ ID NO: 68 - protein, 15 aa, Borrelia burgdorferi
LSLQVVKMKKAISVA

SEQ ID NO: 69 - protein, 15 aa, Borrelia burgdorferi
VVKMKKAISVAISSV

SEQ ID NO: 70 - protein, 15 aa, Borrelia burgdorferi
SVAISSVLRNISYVN

SEQ ID NO: 71 - protein, 15 aa, Borrelia burgdorferi
LRNISYVNKASLEVA

SEQ ID NO: 72 - protein, 15 aa, Borrelia burgdorferi
RNISYVNKASLEVAS

SEQ ID NO: 73 - protein, 15 aa, Borrelia burgdorferi
YVNKASLEVASSSQN

SEQ ID NO: 74 - protein, 15 aa, Borrelia burgdorferi
SSQNLSSSALQQASA

SEQ ID NO: 75 - protein, 15 aa, Borrelia burgdorferi
SSALQQASALEEMSA

SEQ ID NO: 76 - protein, 15 aa, Borrelia burgdorferi
ALEEMSANVEQIASG

SEQ ID NO: 77 - protein, 15 aa, Borrelia burgdorferi
NVEQIASGVNMSANN

SEQ ID NO: 78 - protein, 15 aa, Borrelia burgdorferi
NNSYETEQIALKTNE

SEQ ID NO: 79 - protein, 15 aa, Borrelia burgdorferi
NSYETEQIALKTNEN

SEQ ID NO: 80 - protein, 15 aa, Borrelia burgdorferi
SYETEQIALKTNENS

SEQ ID NO: 81 - protein, 15 aa, Borrelia burgdorferi
ETEQIALKTNENSQI

SEQ ID NO: 82 - protein, 15 aa, Borrelia burgdorferi
TEQIALKTNENSQIG

SEQ ID NO: 83 - protein, 15 aa, Borrelia burgdorferi
EQIALKTNENSQIGG

SEQ ID NO: 84 - protein, 15 aa, Borrelia burgdorferi
RAVEESVIAMQDIVE

SEQ ID NO: 85 - protein, 15 aa, Borrelia burgdorferi
EESVIAMQDIVEKVS

SEQ ID NO: 86 - protein, 15 aa, Borrelia burgdorferi
QDIVEKVSVIEEIAR

SEQ ID NO: 87 - protein, 15 aa, Borrelia burgdorferi
DIVEKVSVIEEIARK

SEQ ID NO: 88 - protein, 15 aa, Borrelia burgdorferi
VEKVSVIEEIARKTN

SEQ ID NO: 89 - protein, 15 aa, Borrelia burgdorferi
VIEEIARKTNLLALN

SEQ ID NO: 90 - protein, 15 aa, Borrelia burgdorferi
KTNLLALNAAIEAAR

SEQ ID NO: 91 - protein, 15 aa, Borrelia burgdorferi
TNLLALNAAIEAARA

SEQ ID NO: 92 - protein, 15 aa, Borrelia burgdorferi
ALNAAIEAARAGDEG

SEQ ID NO: 93 - protein, 15 aa, Borrelia burgdorferi
LNAAIEAARAGDEGK

SEQ ID NO: 94 - protein, 15 aa, Borrelia burgdorferi
NAAIEAARAGDEGKG

SEQ ID NO: 95 - protein, 15 aa, Borrelia burgdorferi
GDEGKGFAVVASEIR

SEQ ID NO: 96 - protein, 15 aa, Borrelia burgdorferi
GKGFAVVASEIRKLA

SEQ ID NO: 97 - protein, 15 aa, Borrelia burgdorferi
KGFAVVASEIRKLAD

SEQ ID NO: 98 - protein, 15 aa, Borrelia burgdorferi
ASEIRKLADLSKISA

SEQ ID NO: 99 - protein, 15 aa, Borrelia burgdorferi
EIRKLADLSKISALE

SEQ ID NO: 100 - protein, 15 aa, Borrelia burgdorferi
IGELVEDNSKVATEA

SEQ ID NO: 101 - protein, 15 aa, Borrelia burgdorferi
TANLVKKISEGSSKQ

SEQ ID NO: 102 - protein, 15 aa, Borrelia burgdorferi
ANLVKKISEGSSKQS

SEQ ID NO: 103 - protein, 15 aa, Borrelia burgdorferi
LDQVGEVVQSSASSS

SEQ ID NO: 104 - protein, 15 aa, Borrelia burgdorferi
DQVGEVVQSSASSSE

SEQ ID NO: 105 - protein, 15 aa, Borrelia burgdorferi
QVGEVVQSSASSSEQ

SEQ ID NO: 106 - protein, 15 aa, Borrelia burgdorferi
SDKMLEKSKELRKSV

SEQ ID NO: 107 - protein, 15 aa, Borrelia burgdorferi
SVLFFKIKDSKIENP

SEQ ID NO: 108 - protein, 15 aa, Borrelia burgdorferi
PENSFKDENQNLKSN

SEQ ID NO: 109 - protein, 15 aa, Borrelia burgdorferi
NLKSNGISTSNASGH

SEQ ID NO: 110 - protein, 15 aa, Borrelia burgdorferi
KSNGISTSNASGHNN

SEQ ID NO: 111 - protein, 15 aa, Borrelia burgdorferi
SESSVRTINKRVDPK

SEQ ID NO: 112 - protein, 15 aa, Borrelia burgdorferi
ESSVRTINKRVDPKK

SEQ ID NO: 113 - protein, 15 aa, Borrelia burgdorferi
VRTINKRVDPKKAID

SEQ ID NO: 114 - protein, 15 aa, Borrelia burgdorferi
KKAIDIADKDLNFDD

SEQ ID NO: 115 - protein, 9 aa, Borrelia burgdorferi
YKQLTRAQV

SEQ ID NO: 116 - protein, 9 aa, Borrelia burgdorferi
LTRAQVRRA

SEQ ID NO: 117 - protein, 9 aa, Borrelia burgdorferi
LHVIINGAA

SEQ ID NO: 118 - protein, 9 aa, Borrelia burgdorferi
INGAASNLA

SEQ ID NO: 119 - protein, 9 aa, Borrelia burgdorferi
FAMSENRGK

SEQ ID NO: 120 - protein, 9 aa, Borrelia burgdorferi
LIDLRKNPK

SEQ ID NO: 121 - protein, 9 aa, Borrelia burgdorferi
FVIDSVKVS

SEQ ID NO: 122 - protein, 9 aa, Borrelia burgdorferi
IDSVKVSKK

SEQ ID NO: 123 - protein, 9 aa, Borrelia burgdorferi
VKVSKKYRQ

SEQ ID NO: 124 - protein, 9 aa, Borrelia burgdorferi
LYNFMANLK

SEQ ID NO: 125 - protein, 9 aa, Borrelia burgdorferi
VIVSTRHEN

SEQ ID NO: 126 - protein, 9 aa, Borrelia burgdorferi
FGHSEANTN

SEQ ID NO: 127 - protein, 9 aa, Borrelia burgdorferi
YFKKAVEDY

SEQ ID NO: 128 - protein, 9 aa, Borrelia burgdorferi
FKKAVEDYR

SEQ ID NO: 129 - protein, 9 aa, Borrelia burgdorferi
SEGISAEVA

SEQ ID NO: 130 - protein, 9 aa, Borrelia burgdorferi
ISAEVAIRS

SEQ ID NO: 131 - protein, 9 aa, Borrelia burgdorferi
VAIRSKQSE

SEQ ID NO: 132 - protein, 9 aa, Borrelia burgdorferi
EVAIRSKQS

SEQ ID NO: 133 - protein, 9 aa, Borrelia burgdorferi
IRSKQSEKK

SEQ ID NO: 134 - protein, 9 aa, Borrelia burgdorferi
KAFAIIVPV

SEQ ID NO: 135 - protein, 9 aa, Borrelia burgdorferi
FAIIVPVYS

SEQ ID NO: 136 - protein, 9 aa, Borrelia burgdorferi
FIYVDPNNI

SEQ ID NO: 137 - protein, 9 aa, Borrelia burgdorferi
IYVDPNNIA

SEQ ID NO: 138 - protein, 9 aa, Borrelia burgdorferi
YVDPNNIAV

SEQ ID NO: 139 - protein, 9 aa, Borrelia burgdorferi
LNVLKDVFS

SEQ ID NO: 140 - protein, 9 aa, Borrelia burgdorferi
YTIDRILLS

SEQ ID NO: 141 - protein, 9 aa, Borrelia burgdorferi
YYAMLPISS

SEQ ID NO: 142 - protein, 9 aa, Borrelia burgdorferi
VLIARLPYK

SEQ ID NO: 143 - protein, 9 aa, Borrelia burgdorferi
IYGVISSLR

SEQ ID NO: 144 - protein, 9 aa, Borrelia burgdorferi
FRLNAIRVL

SEQ ID NO: 145 - protein, 9 aa, Borrelia burgdorferi
IRVLVQDMV

SEQ ID NO: 146 - protein, 9 aa, Borrelia burgdorferi
VVKMKKAIS

SEQ ID NO: 147 - protein, 9 aa, Borrelia burgdorferi
KKAISVAIS

SEQ ID NO: 148 - protein, 9 aa, Borrelia burgdorferi
ISSVLRNIS

SEQ ID NO: 149 - protein, 9 aa, Borrelia burgdorferi
ISYVNKASL

SEQ ID NO: 150 - protein, 9 aa, Borrelia burgdorferi
YVNKASLEV

SEQ ID NO: 151 - protein, 9 aa, Borrelia burgdorferi
KASLEVASS

SEQ ID NO: 152 - protein, 9 aa, Borrelia burgdorferi
LSSSALQQA

SEQ ID NO: 153 - protein, 9 aa, Borrelia burgdorferi
LQQASALEE

SEQ ID NO: 154 - protein, 9 aa, Borrelia burgdorferi
EMSANVEQI

SEQ ID NO: 155 - protein, 9 aa, Borrelia burgdorferi
QIASGVNMS

SEQ ID NO: 156 - protein, 9 aa, Borrelia burgdorferi
YETEQIALK

SEQ ID NO: 157 - protein, 9 aa, Borrelia burgdorferi
ETEQIALKT

SEQ ID NO: 158 - protein, 9 aa, Borrelia burgdorferi
QIALKTNEN

SEQ ID NO: 159 - protein, 9 aa, Borrelia burgdorferi
IALKTNENS

SEQ ID NO: 160 - protein, 9 aa, Borrelia burgdorferi
LKTNENSQI

SEQ ID NO: 161 - protein, 9 aa, Borrelia burgdorferi
EESVIAMQD

SEQ ID NO: 162 - protein, 9 aa, Borrelia burgdorferi
IAMQDIVEK

SEQ ID NO: 163 - protein, 9 aa, Borrelia burgdorferi
VEKVSVIEE

SEQ ID NO: 164 - protein, 9 aa, Borrelia burgdorferi
EKVSVIEEI

SEQ ID NO: 165 - protein, 9 aa, Borrelia burgdorferi
VSVIEEIAR

SEQ ID NO: 166 - protein, 9 aa, Borrelia burgdorferi
IARKTNLLA

SEQ ID NO: 167 - protein, 9 aa, Borrelia burgdorferi
LLALNAAIE

SEQ ID NO: 168 - protein, 9 aa, Borrelia burgdorferi
LALNAAIEA

SEQ ID NO: 169 - protein, 9 aa, Borrelia burgdorferi
NAAIEAARA

SEQ ID NO: 170 - protein, 9 aa, Borrelia burgdorferi
IEAARAGDE

SEQ ID NO: 171 - protein, 9 aa, Borrelia burgdorferi
GKGFAVVAS

SEQ ID NO: 172 - protein, 9 aa, Borrelia burgdorferi
FAVVASEIR

SEQ ID NO: 173 - protein, 9 aa, Borrelia burgdorferi
VASEIRKLA

SEQ ID NO: 174 - protein, 9 aa, Borrelia burgdorferi
VVASEIRKL

SEQ ID NO: 175 - protein, 9 aa, Borrelia burgdorferi
IRKLADLSK

SEQ ID NO: 176 - protein, 9 aa, Borrelia burgdorferi
LADLSKISA

SEQ ID NO: 177 - protein, 9 aa, Borrelia burgdorferi
LVEDNSKVA

SEQ ID NO: 178 - protein, 9 aa, Borrelia burgdorferi
LVKKISEGS

SEQ ID NO: 179 - protein, 9 aa, Borrelia burgdorferi
VKKISEGSS

SEQ ID NO: 180 - protein, 9 aa, Borrelia burgdorferi
QVGEVVQSS

SEQ ID NO: 181 - protein, 9 aa, Borrelia burgdorferi
VVQSSASSS

SEQ ID NO: 182 - protein, 9 aa, Borrelia burgdorferi
LEKSKELRK

SEQ ID NO: 183 - protein, 9 aa, Borrelia burgdorferi
FKIKDSKIE

SEQ ID NO: 184 - protein, 9 aa, Borrelia burgdorferi
FKDENQNLK

SEQ ID NO: 185 - protein, 9 aa, Borrelia burgdorferi
SNGISTSNA

SEQ ID NO: 186 - protein, 9 aa, Borrelia burgdorferi
ISTSNASGH

SEQ ID NO: 187 - protein, 9 aa, Borrelia burgdorferi
VRTINKRVD

SEQ ID NO: 188 - protein, 9 aa, Borrelia burgdorferi
INKRVDPKK

SEQ ID NO: 189 - protein, 9 aa, Borrelia burgdorferi
IDIADKDLN

SEQ ID NO: 190 - protein, 15 aa, Borrelia burgdorferi
LEDYYKQLTRAQVRR

SEQ ID NO: 191 - protein, 15 aa, Borrelia burgdorferi
EDYYKQLTRAQVRRA

SEQ ID NO: 192 - protein, 15 aa, Borrelia burgdorferi
YKQLTRAQVRRAAFS

SEQ ID NO: 193 - protein, 15 aa, Borrelia burgdorferi
FLDTLHVIINGAASN

SEQ ID NO: 194 - protein, 15 aa, Borrelia burgdorferi
DTLHVIINGAASNLA

SEQ ID NO: 195 - protein, 15 aa, Borrelia burgdorferi
TLHVIINGAASNLAL

SEQ ID NO: 196 - protein, 15 aa, Borrelia burgdorferi
LHVIINGAASNLALE

SEQ ID NO: 197 - protein, 15 aa, Borrelia burgdorferi
HVIINGAASNLALET

SEQ ID NO: 198 - protein, 15 aa, Borrelia burgdorferi
NLALETISEFAMSEN

SEQ ID NO: 199 - protein, 15 aa, Borrelia burgdorferi
ISEFAMSENRGKDFS

SEQ ID NO: 200 - protein, 15 aa, Borrelia burgdorferi
SESELIDLRKNPKFV

SEQ ID NO: 201 - protein, 15 aa, Borrelia burgdorferi
ESELIDLRKNPKFVI

SEQ ID NO: 202 - protein, 15 aa, Borrelia burgdorferi
SELIDLRKNPKFVID

SEQ ID NO: 203 - protein, 15 aa, Borrelia burgdorferi
ELIDLRKNPKFVIDS

SEQ ID NO: 204 - protein, 15 aa, Borrelia burgdorferi
LIDLRKNPKFVIDSV

SEQ ID NO: 205 - protein, 15 aa, Borrelia burgdorferi
KNPKFVIDSVKVSKK

SEQ ID NO: 206 - protein, 15 aa, Borrelia burgdorferi
NPKFVIDSVKVSKKY

SEQ ID NO: 207 - protein, 15 aa, Borrelia burgdorferi
PKFVIDSVKVSKKYR

SEQ ID NO: 208 - protein, 15 aa, Borrelia burgdorferi
KFVIDSVKVSKKYRQ

SEQ ID NO: 209 - protein, 15 aa, Borrelia burgdorferi
VIDSVKVSKKYRQYL

SEQ ID NO: 210 - protein, 15 aa, Borrelia burgdorferi
IDSVKVSKKYRQYLY

SEQ ID NO: 211 - protein, 15 aa, Borrelia burgdorferi
SVKVSKKYRQYLYNF

SEQ ID NO: 212 - protein, 15 aa, Borrelia burgdorferi
YRQYLYNFMANLKND

SEQ ID NO: 213 - protein, 15 aa, Borrelia burgdorferi
RQYLYNFMANLKNDT

SEQ ID NO: 214 - protein, 15 aa, Borrelia burgdorferi
QYLYNFMANLKNDTL

SEQ ID NO: 215 - protein, 15 aa, Borrelia burgdorferi
EFAFFDFEGRVIVST

SEQ ID NO: 216 - protein, 15 aa, Borrelia burgdorferi
EGRVIVSTRHENNMD

SEQ ID NO: 217 - protein, 15 aa, Borrelia burgdorferi
GRVIVSTRHENNMDF

SEQ ID NO: 218 - protein, 15 aa, Borrelia burgdorferi
NMDFGHSEANTNYFK

SEQ ID NO: 219 - protein, 15 aa, Borrelia burgdorferi
EANTNYFKKAVEDYR

SEQ ID NO: 220 - protein, 15 aa, Borrelia burgdorferi
ANTNYFKKAVEDYRQ

SEQ ID NO: 221 - protein, 15 aa, Borrelia burgdorferi
TNYFKKAVEDYRQNQ

SEQ ID NO: 222 - protein, 15 aa, Borrelia burgdorferi
FKKAVEDYRQNQLKF

SEQ ID NO: 223 - protein, 15 aa, Borrelia burgdorferi
KKAVEDYRQNQLKFI

SEQ ID NO: 224 - protein, 15 aa, Borrelia burgdorferi
VEDYRQNQLKFIGWY

SEQ ID NO: 225 - protein, 15 aa, Borrelia burgdorferi
NQLKFIGWYSNLSEG

SEQ ID NO: 226 - protein, 15 aa, Borrelia burgdorferi
QLKFIGWYSNLSEGI

SEQ ID NO: 227 - protein, 15 aa, Borrelia burgdorferi
LKFIGWYSNLSEGIS

SEQ ID NO: 228 - protein, 15 aa, Borrelia burgdorferi
KFIGWYSNLSEGISA

SEQ ID NO: 229 - protein, 15 aa, Borrelia burgdorferi
FIGWYSNLSEGISAE

SEQ ID NO: 230 - protein, 15 aa, Borrelia burgdorferi
LSEGISAEVAIRSKQ

SEQ ID NO: 231 - protein, 15 aa, Borrelia burgdorferi
SEGISAEVAIRSKQS

SEQ ID NO: 232 - protein, 15 aa, Borrelia burgdorferi
SAEVAIRSKQSEKKA

SEQ ID NO: 233 - protein, 15 aa, Borrelia burgdorferi
EVAIRSKQSEKKAFA

SEQ ID NO: 234 - protein, 15 aa, Borrelia burgdorferi
EKKAFAIIVPVYSPE

SEQ ID NO: 235 - protein, 15 aa, Borrelia burgdorferi
KKAFAIIVPVYSPED

SEQ ID NO: 236 - protein, 15 aa, Borrelia burgdorferi
KAFAIIVPVYSPEDK

SEQ ID NO: 237 - protein, 15 aa, Borrelia burgdorferi
AFAIIVPVYSPEDKL

SEQ ID NO: 238 - protein, 15 aa, Borrelia burgdorferi
IVPVYSPEDKLVCGY

SEQ ID NO: 239 - protein, 15 aa, Borrelia burgdorferi
LAGYLLNDIVADSFD

SEQ ID NO: 240 - protein, 15 aa, Borrelia burgdorferi
AGYLLNDIVADSFDR

SEQ ID NO: 241 - protein, 15 aa, Borrelia burgdorferi
GYLLNDIVADSFDRF

SEQ ID NO: 242 - protein, 15 aa, Borrelia burgdorferi
LNDIVADSFDRFRFG

SEQ ID NO: 243 - protein, 15 aa, Borrelia burgdorferi
YKRGNFIYVDPNNIA

SEQ ID NO: 244 - protein, 15 aa, Borrelia burgdorferi
KRGNFIYVDPNNIAV

SEQ ID NO: 245 - protein, 15 aa, Borrelia burgdorferi
RGNFIYVDPNNIAVN

SEQ ID NO: 246 - protein, 15 aa, Borrelia burgdorferi
GNFIYVDPNNIAVNP

SEQ ID NO: 247 - protein, 15 aa, Borrelia burgdorferi
FIYVDPNNIAVNPFE

SEQ ID NO: 248 - protein, 15 aa, Borrelia burgdorferi
DPNNIAVNPFEEYNE

SEQ ID NO: 249 - protein, 15 aa, Borrelia burgdorferi
PNNIAVNPFEEYNET

SEQ ID NO: 250 - protein, 15 aa, Borrelia burgdorferi
VNPFEEYNETSRVSS

SEQ ID NO: 251 - protein, 15 aa, Borrelia burgdorferi
PFEEYNETSRVSSKF

SEQ ID NO: 252 - protein, 15 aa, Borrelia burgdorferi
SSKFLNVLKDVFSKP

SEQ ID NO: 253 - protein, 15 aa, Borrelia burgdorferi
SKFLNVLKDVFSKPP

SEQ ID NO: 254 - protein, 15 aa, Borrelia burgdorferi
KPPFPSNIASEVSVY

SEQ ID NO: 255 - protein, 15 aa, Borrelia burgdorferi
PSNIASEVSVYTIDR

SEQ ID NO: 256 - protein, 15 aa, Borrelia burgdorferi
ASEVSVYTIDRILLS

SEQ ID NO: 257 - protein, 15 aa, Borrelia burgdorferi
SEVSVYTIDRILLSE

SEQ ID NO: 258 - protein, 15 aa, Borrelia burgdorferi
EVSVYTIDRILLSEM

SEQ ID NO: 259 - protein, 15 aa, Borrelia burgdorferi
VSVYTIDRILLSEMG

SEQ ID NO: 260 - protein, 15 aa, Borrelia burgdorferi
EDCYYAMLPISSKLG

SEQ ID NO: 261 - protein, 15 aa, Borrelia burgdorferi
YYAMLPISSKLGEKS

SEQ ID NO: 262 - protein, 15 aa, Borrelia burgdorferi
ISSKLGEKSGVLIAR

SEQ ID NO: 263 - protein, 15 aa, Borrelia burgdorferi
GEKSGVLIARLPYKD

SEQ ID NO: 264 - protein, 15 aa, Borrelia burgdorferi
EKSGVLIARLPYKDI

SEQ ID NO: 265 - protein, 15 aa, Borrelia burgdorferi
KSGVLIARLPYKDIY

SEQ ID NO: 266 - protein, 15 aa, Borrelia burgdorferi
SGVLIARLPYKDIYG

SEQ ID NO: 267 - protein, 15 aa, Borrelia burgdorferi
ARLPYKDIYGVISSL

SEQ ID NO: 268 - protein, 15 aa, Borrelia burgdorferi
RLPYKDIYGVISSLR

SEQ ID NO: 269 - protein, 15 aa, Borrelia burgdorferi
PYKDIYGVISSLRFQ

SEQ ID NO: 270 - protein, 15 aa, Borrelia burgdorferi
YKDIYGVISSLRFQY

SEQ ID NO: 271 - protein, 15 aa, Borrelia burgdorferi
KDIYGVISSLRFQYI

SEQ ID NO: 272 - protein, 15 aa, Borrelia burgdorferi
ISSLRFQYILYSVLG

SEQ ID NO: 273 - protein, 15 aa, Borrelia burgdorferi
GIIALSIVLSIRIDR

SEQ ID NO: 274 - protein, 15 aa, Borrelia burgdorferi
ALSIVLSIRIDRIIS

SEQ ID NO: 275 - protein, 15 aa, Borrelia burgdorferi
LSIVLSIRIDRIISF

SEQ ID NO: 276 - protein, 15 aa, Borrelia burgdorferi
IVLSIRIDRIISFRL

SEQ ID NO: 277 - protein, 15 aa, Borrelia burgdorferi
VLSIRIDRIISFRLN

SEQ ID NO: 278 - protein, 15 aa, Borrelia burgdorferi
LSIRIDRIISFRLNA

SEQ ID NO: 279 - protein, 15 aa, Borrelia burgdorferi
SIRIDRIISFRLNAI

SEQ ID NO: 280 - protein, 15 aa, Borrelia burgdorferi
IRIDRIISFRLNAIR

SEQ ID NO: 281 - protein, 15 aa, Borrelia burgdorferi
RIDRIISFRLNAIRV

SEQ ID NO: 282 - protein, 15 aa, Borrelia burgdorferi
IISFRLNAIRVLVQD

SEQ ID NO: 283 - protein, 15 aa, Borrelia burgdorferi
LNAIRVLVQDMVKGN

SEQ ID NO: 284 - protein, 15 aa, Borrelia burgdorferi
NAIRVLVQDMVKGNL

SEQ ID NO: 285 - protein, 15 aa, Borrelia burgdorferi
AIRVLVQDMVKGNLD

SEQ ID NO: 286 - protein, 15 aa, Borrelia burgdorferi
RVLVQDMVKGNLDKD

SEQ ID NO: 287 - protein, 15 aa, Borrelia burgdorferi
LVQDMVKGNLDKDYA

SEQ ID NO: 288 - protein, 15 aa, Borrelia burgdorferi
KGNLDKDYALDDDEN

SEQ ID NO: 289 - protein, 15 aa, Borrelia burgdorferi
GNLDKDYALDDDENT

SEQ ID NO: 290 - protein, 15 aa, Borrelia burgdorferi
DKDYALDDDENTLDE

SEQ ID NO: 291 - protein, 15 aa, Borrelia burgdorferi
DELGMLSLQVVKMKK

SEQ ID NO: 292 - protein, 15 aa, Borrelia burgdorferi
ELGMLSLQVVKMKKA

SEQ ID NO: 293 - protein, 15 aa, Borrelia burgdorferi
LSLQVVKMKKAISVA

SEQ ID NO: 294 - protein, 15 aa, Borrelia burgdorferi
SLQVVKMKKAISVAI

SEQ ID NO: 295 - protein, 15 aa, Borrelia burgdorferi
LQVVKMKKAISVAIS

SEQ ID NO: 296 - protein, 15 aa, Borrelia burgdorferi
QVVKMKKAISVAISS

SEQ ID NO: 297 - protein, 15 aa, Borrelia burgdorferi
VVKMKKAISVAISSV

SEQ ID NO: 298 - protein, 15 aa, Borrelia burgdorferi
VKMKKAISVAISSVL

SEQ ID NO: 299 - protein, 15 aa, Borrelia burgdorferi
KMKKAISVAISSVLR

SEQ ID NO: 300 - protein, 15 aa, Borrelia burgdorferi
MKKAISVAISSVLRN

SEQ ID NO: 301 - protein, 15 aa, Borrelia burgdorferi
ISVAISSVLRNISYV

SEQ ID NO: 302 - protein, 15 aa, Borrelia burgdorferi
SVAISSVLRNISYVN

SEQ ID NO: 303 - protein, 15 aa, Borrelia burgdorferi
ISSVLRNISYVNKAS

SEQ ID NO: 304 - protein, 15 aa, Borrelia burgdorferi
VLRNISYVNKASLEV

SEQ ID NO: 305 - protein, 15 aa, Borrelia burgdorferi
LRNISYVNKASLEVA

SEQ ID NO: 306 - protein, 15 aa, Borrelia burgdorferi
RNISYVNKASLEVAS

SEQ ID NO: 307 - protein, 15 aa, Borrelia burgdorferi
NISYVNKASLEVASS

SEQ ID NO: 308 - protein, 15 aa, Borrelia burgdorferi
KASLEVASSSQNLSS

SEQ ID NO: 309 - protein, 15 aa, Borrelia burgdorferi
ASALEEMSANVEQIA

SEQ ID NO: 310 - protein, 15 aa, Borrelia burgdorferi
VEQIASGVNMSANNS

SEQ ID NO: 311 - protein, 15 aa, Borrelia burgdorferi
ASGVNMSANNSYETE

SEQ ID NO: 312 - protein, 15 aa, Borrelia burgdorferi
SGVNMSANNSYETEQ

SEQ ID NO: 313 - protein, 15 aa, Borrelia burgdorferi
NNSYETEQIALKTNE

SEQ ID NO: 314 - protein, 15 aa, Borrelia burgdorferi
TEQIALKTNENSQIG

SEQ ID NO: 315 - protein, 15 aa, Borrelia burgdorferi
QIALKTNENSQIGGR

SEQ ID NO: 316 - protein, 15 aa, Borrelia burgdorferi
VEESVIAMQDIVEKV

SEQ ID NO: 317 - protein, 15 aa, Borrelia burgdorferi
EESVIAMQDIVEKVS

SEQ ID NO: 318 - protein, 15 aa, Borrelia burgdorferi
AMQDIVEKVSVIEEI

SEQ ID NO: 319 - protein, 15 aa, Borrelia burgdorferi
MQDIVEKVSVIEEIA

SEQ ID NO: 320 - protein, 15 aa, Borrelia burgdorferi
QDIVEKVSVIEEIAR

SEQ ID NO: 321 - protein, 15 aa, Borrelia burgdorferi
VEKVSVIEEIARKTN

SEQ ID NO: 322 - protein, 15 aa, Borrelia burgdorferi
EKVSVIEEIARKTNL

SEQ ID NO: 323 - protein, 15 aa, Borrelia burgdorferi
VSVIEEIARKTNLLA

SEQ ID NO: 324 - protein, 15 aa, Borrelia burgdorferi
SVIEEIARKTNLLAL

SEQ ID NO: 325 - protein, 15 aa, Borrelia burgdorferi
VIEEIARKTNLLALN

SEQ ID NO: 326 - protein, 15 aa, Borrelia burgdorferi
IEEIARKTNLLALNA

SEQ ID NO: 327 - protein, 15 aa, Borrelia burgdorferi
RKTNLLALNAAIEAA

SEQ ID NO: 328 - protein, 15 aa, Borrelia burgdorferi
KTNLLALNAAIEAAR

SEQ ID NO: 329 - protein, 15 aa, Borrelia burgdorferi
TNLLALNAAIEAARA

SEQ ID NO: 330 - protein, 15 aa, Borrelia burgdorferi
LLALNAAIEAARAGD

SEQ ID NO: 331 - protein, 15 aa, Borrelia burgdorferi
EGKGFAVVASEIRKL

SEQ ID NO: 332 - protein, 15 aa, Borrelia burgdorferi
GKGFAVVASEIRKLA

SEQ ID NO: 333 - protein, 15 aa, Borrelia burgdorferi
KGFAVVASEIRKLAD

SEQ ID NO: 334 - protein, 15 aa, Borrelia burgdorferi
FAVVASEIRKLADLS

SEQ ID NO: 335 - protein, 15 aa, Borrelia burgdorferi
VASEIRKLADLSKIS

SEQ ID NO: 336 - protein, 15 aa, Borrelia burgdorferi
ASEIRKLADLSKISA

SEQ ID NO: 337 - protein, 15 aa, Borrelia burgdorferi
EIRKLADLSKISALE

SEQ ID NO: 338 - protein, 15 aa, Borrelia burgdorferi
KLADLSKISALEIGE

SEQ ID NO: 339 - protein, 15 aa, Borrelia burgdorferi
LADLSKISALEIGEL

SEQ ID NO: 340 - protein, 15 aa, Borrelia burgdorferi
LSKISALEIGELVED

SEQ ID NO: 341 - protein, 15 aa, Borrelia burgdorferi
ALEIGELVEDNSKVA

SEQ ID NO: 342 - protein, 15 aa, Borrelia burgdorferi
IGELVEDNSKVATEA

SEQ ID NO: 343 - protein, 15 aa, Borrelia burgdorferi
GELVEDNSKVATEAG

SEQ ID NO: 344 - protein, 15 aa, Borrelia burgdorferi
DNSKVATEAGVIFKE

SEQ ID NO: 345 - protein, 15 aa, Borrelia burgdorferi
EAGVIFKEMLPEIEE

SEQ ID NO: 346 - protein, 15 aa, Borrelia burgdorferi
AGVIFKEMLPEIEET

SEQ ID NO: 347 - protein, 15 aa, Borrelia burgdorferi
GVIFKEMLPEIEETA

SEQ ID NO: 348 - protein, 15 aa, Borrelia burgdorferi
LPEIEETANLVKKIS

SEQ ID NO: 349 - protein, 15 aa, Borrelia burgdorferi
TANLVKKISEGSSKQ

SEQ ID NO: 350 - protein, 15 aa, Borrelia burgdorferi
SDQIAQFKMALDQVG

SEQ ID NO: 351 - protein, 15 aa, Borrelia burgdorferi
QIAQFKMALDQVGEV

SEQ ID NO: 352 - protein, 15 aa, Borrelia burgdorferi
IAQFKMALDQVGEVV

SEQ ID NO: 353 - protein, 15 aa, Borrelia burgdorferi
QFKMALDQVGEVVQS

SEQ ID NO: 354 - protein, 15 aa, Borrelia burgdorferi
SEQLSSMSDKMLEKS

SEQ ID NO: 355 - protein, 15 aa, Borrelia burgdorferi
SDKMLEKSKELRKSV

SEQ ID NO: 356 - protein, 15 aa, Borrelia burgdorferi
RKSVLFFKIKDSKIE

SEQ ID NO: 357 - protein, 15 aa, Borrelia burgdorferi
KSVLFFKIKDSKIEN

SEQ ID NO: 358 - protein, 15 aa, Borrelia burgdorferi
SVLFFKIKDSKIENP

SEQ ID NO: 359 - protein, 15 aa, Borrelia burgdorferi
VLFFKIKDSKIENPE

SEQ ID NO: 360 - protein, 15 aa, Borrelia burgdorferi
DYDFRLIDCPENSFK

SEQ ID NO: 361 - protein, 15 aa, Borrelia burgdorferi
ENSFKDENQNLKSNG

SEQ ID NO: 362 - protein, 15 aa, Borrelia burgdorferi
NQNLKSNGISTSNAS

SEQ ID NO: 363 - protein, 15 aa, Borrelia burgdorferi
HNNYSLDIESESSVR

SEQ ID NO: 364 - protein, 15 aa, Borrelia burgdorferi
SLDIESESSVRTINK

SEQ ID NO: 365 - protein, 15 aa, Borrelia burgdorferi
ESSVRTINKRVDPKK

SEQ ID NO: 366 - protein, 15 aa, Borrelia burgdorferi
VRTINKRVDPKKAID

SEQ ID NO: 367 - protein, 15 aa, Borrelia burgdorferi
NKRVDPKKAIDIADK

SEQ ID NO: 368 - protein, 15 aa, Borrelia burgdorferi
KKAIDIADKDLNFDD

SEQ ID NO: 369 - protein, 15 aa, Borrelia burgdorferi
KAIDIADKDLNFDDD

SEQ ID NO: 370 - protein, 9 aa, Borrelia burgdorferi
YKQLTRAQV

SEQ ID NO: 371 - protein, 9 aa, Borrelia burgdorferi
YYKQLTRAQ

SEQ ID NO: 372 - protein, 9 aa, Borrelia burgdorferi
LTRAQVRRA

SEQ ID NO: 373 - protein, 9 aa, Borrelia burgdorferi
LHVIINGAA

SEQ ID NO: 374 - protein, 9 aa, Borrelia burgdorferi
VIINGAASN

SEQ ID NO: 375 - protein, 9 aa, Borrelia burgdorferi
INGAASNLA

SEQ ID NO: 376 - protein, 9 aa, Borrelia burgdorferi
LETISEFAM

SEQ ID NO: 377 - protein, 9 aa, Borrelia burgdorferi
FAMSENRGK

SEQ ID NO: 378 - protein, 9 aa, Borrelia burgdorferi
LIDLRKNPK

SEQ ID NO: 379 - protein, 9 aa, Borrelia burgdorferi
IDLRKNPKF

SEQ ID NO: 380 - protein, 9 aa, Borrelia burgdorferi
LRKNPKFVI

SEQ ID NO: 381 - protein, 9 aa, Borrelia burgdorferi
FVIDSVKVS

SEQ ID NO: 382 - protein, 9 aa, Borrelia burgdorferi
IDSVKVSKK

SEQ ID NO: 383 - protein, 9 aa, Borrelia burgdorferi
VIDSVKVSK

SEQ ID NO: 384 - protein, 9 aa, Borrelia burgdorferi
VKVSKKYRQ

SEQ ID NO: 385 - protein, 9 aa, Borrelia burgdorferi
VSKKYRQYL

SEQ ID NO: 386 - protein, 9 aa, Borrelia burgdorferi
YLYNFMANL

SEQ ID NO: 387 - protein, 9 aa, Borrelia burgdorferi
LYNFMANLK

SEQ ID NO: 388 - protein, 9 aa, Borrelia burgdorferi
YNFMANLKN

SEQ ID NO: 389 - protein, 9 aa, Borrelia burgdorferi
FFDFEGRVI

SEQ ID NO: 390 - protein, 9 aa, Borrelia burgdorferi
VIVSTRHEN

SEQ ID NO: 391 - protein, 9 aa, Borrelia burgdorferi
IVSTRHENN

SEQ ID NO: 392 - protein, 9 aa, Borrelia burgdorferi
FGHSEANTN

SEQ ID NO: 393 - protein, 9 aa, Borrelia burgdorferi
TNYFKKAVE

SEQ ID NO: 394 - protein, 9 aa, Borrelia burgdorferi
YFKKAVEDY

SEQ ID NO: 395 - protein, 9 aa, Borrelia burgdorferi
FKKAVEDYR

SEQ ID NO: 396 - protein, 9 aa, Borrelia burgdorferi
VEDYRQNQL

SEQ ID NO: 397 - protein, 9 aa, Borrelia burgdorferi
YRQNQLKFI

SEQ ID NO: 398 - protein, 9 aa, Borrelia burgdorferi
FIGWYSNLS

SEQ ID NO: 399 - protein, 9 aa, Borrelia burgdorferi
IGWYSNLSE

SEQ ID NO: 400 - protein, 9 aa, Borrelia burgdorferi
WYSNLSEGI

SEQ ID NO: 401 - protein, 9 aa, Borrelia burgdorferi
ISAEVAIRS

SEQ ID NO: 402 - protein, 9 aa, Borrelia burgdorferi
VAIRSKQSE

SEQ ID NO: 403 - protein, 9 aa, Borrelia burgdorferi
IRSKQSEKK

SEQ ID NO: 404 - protein, 9 aa, Borrelia burgdorferi
AFAIIVPVY

SEQ ID NO: 405 - protein, 9 aa, Borrelia burgdorferi
FAIIVPVYS

SEQ ID NO: 406 - protein, 9 aa, Borrelia burgdorferi
IVPVYSPED

SEQ ID NO: 407 - protein, 9 aa, Borrelia burgdorferi
YSPEDKLVC

SEQ ID NO: 408 - protein, 9 aa, Borrelia burgdorferi
YLLNDIVAD

SEQ ID NO: 409 - protein, 9 aa, Borrelia burgdorferi
LLNDIVADS

SEQ ID NO: 410 - protein, 9 aa, Borrelia burgdorferi
IVADSFDRF

SEQ ID NO: 411 - protein, 9 aa, Borrelia burgdorferi
FIYVDPNNI

SEQ ID NO: 412 - protein, 9 aa, Borrelia burgdorferi
IYVDPNNIA

SEQ ID NO: 413 - protein, 9 aa, Borrelia burgdorferi
VDPNNIAVN

SEQ ID NO: 414 - protein, 9 aa, Borrelia burgdorferi
IAVNPFEEY

SEQ ID NO: 415 - protein, 9 aa, Borrelia burgdorferi
FEEYNETSR

SEQ ID NO: 416 - protein, 9 aa, Borrelia burgdorferi
YNETSRVSS

SEQ ID NO: 417 - protein, 9 aa, Borrelia burgdorferi
FLNVLKDVF

SEQ ID NO: 418 - protein, 9 aa, Borrelia burgdorferi
LNVLKDVFS

SEQ ID NO: 419 - protein, 9 aa, Borrelia burgdorferi
FPSNIASEV

SEQ ID NO: 420 - protein, 9 aa, Borrelia burgdorferi
IASEVSVYT

SEQ ID NO: 421 - protein, 9 aa, Borrelia burgdorferi
VSVYTIDRI

SEQ ID NO: 422 - protein, 9 aa, Borrelia burgdorferi
VYTIDRILL

SEQ ID NO: 423 - protein, 9 aa, Borrelia burgdorferi
YTIDRILLS

SEQ ID NO: 424 - protein, 9 aa, Borrelia burgdorferi
YYAMLPISS

SEQ ID NO: 425 - protein, 9 aa, Borrelia burgdorferi
MLPISSKLG

SEQ ID NO: 426 - protein, 9 aa, Borrelia burgdorferi
LGEKSGVLI

SEQ ID NO: 427 - protein, 9 aa, Borrelia burgdorferi
GVLIARLPY

SEQ ID NO: 428 - protein, 9 aa, Borrelia burgdorferi
IARLPYKDI

SEQ ID NO: 429 - protein, 9 aa, Borrelia burgdorferi
LIARLPYKD

SEQ ID NO: 430 - protein, 9 aa, Borrelia burgdorferi
YKDIYGVIS

SEQ ID NO: 431 - protein, 9 aa, Borrelia burgdorferi
IYGVISSLR

SEQ ID NO: 432 - protein, 9 aa, Borrelia burgdorferi
YGVISSLRF

SEQ ID NO: 433 - protein, 9 aa, Borrelia burgdorferi
VISSLRFQY

SEQ ID NO: 434 - protein, 9 aa, Borrelia burgdorferi
LRFQYILYS

SEQ ID NO: 435 - protein, 9 aa, Borrelia burgdorferi
LSIVLSIRI

SEQ ID NO: 436 - protein, 9 aa, Borrelia burgdorferi
IVLSIRIDR

SEQ ID NO: 437 - protein, 9 aa, Borrelia burgdorferi
VLSIRIDRI

SEQ ID NO: 438 - protein, 9 aa, Borrelia burgdorferi
IRIDRIISF

SEQ ID NO: 439 - protein, 9 aa, Borrelia burgdorferi
IDRIISFRL

SEQ ID NO: 440 - protein, 9 aa, Borrelia burgdorferi
IISFRLNAI

SEQ ID NO: 441 - protein, 9 aa, Borrelia burgdorferi
ISFRLNAIR

SEQ ID NO: 442 - protein, 9 aa, Borrelia burgdorferi
FRLNAIRVL

SEQ ID NO: 443 - protein, 9 aa, Borrelia burgdorferi
IRVLVQDMV

SEQ ID NO: 444 - protein, 9 aa, Borrelia burgdorferi
VLVQDMVKG

SEQ ID NO: 445 - protein, 9 aa, Borrelia burgdorferi
VQDMVKGNL

SEQ ID NO: 446 - protein, 9 aa, Borrelia burgdorferi
MVKGNLDKD

SEQ ID NO: 447 - protein, 9 aa, Borrelia burgdorferi
LDKDYALDD

SEQ ID NO: 448 - protein, 9 aa, Borrelia burgdorferi
YALDDDENT

SEQ ID NO: 449 - protein, 9 aa, Borrelia burgdorferi
MLSLQVVKM

SEQ ID NO: 450 - protein, 9 aa, Borrelia burgdorferi
VVKMKKAIS

SEQ ID NO: 451 - protein, 9 aa, Borrelia burgdorferi
VKMKKAISV

SEQ ID NO: 452 - protein, 9 aa, Borrelia burgdorferi
MKKAISVAI

SEQ ID NO: 453 - protein, 9 aa, Borrelia burgdorferi
ISVAISSVL

SEQ ID NO: 454 - protein, 9 aa, Borrelia burgdorferi
ISSVLRNIS

SEQ ID NO: 455 - protein, 9 aa, Borrelia burgdorferi
VLRNISYVN

SEQ ID NO: 456 - protein, 9 aa, Borrelia burgdorferi
ISYVNKASL

SEQ ID NO: 457 - protein, 9 aa, Borrelia burgdorferi
YVNKASLEV

SEQ ID NO: 458 - protein, 9 aa, Borrelia burgdorferi
LEVASSSQN

SEQ ID NO: 459 - protein, 9 aa, Borrelia burgdorferi
LEEMSANVE

SEQ ID NO: 460 - protein, 9 aa, Borrelia burgdorferi
IASGVNMSA

SEQ ID NO: 461 - protein, 9 aa, Borrelia burgdorferi
VNMSANNSY

SEQ ID NO: 462 - protein, 9 aa, Borrelia burgdorferi
MSANNSYET

SEQ ID NO: 463 - protein, 9 aa, Borrelia burgdorferi
YETEQIALK

SEQ ID NO: 464 - protein, 9 aa, Borrelia burgdorferi
IALKTNENS

SEQ ID NO: 465 - protein, 9 aa, Borrelia burgdorferi
LKTNENSQI

SEQ ID NO: 466 - protein, 9 aa, Borrelia burgdorferi
VIAMQDIVE

SEQ ID NO: 467 - protein, 9 aa, Borrelia burgdorferi
IAMQDIVEK

SEQ ID NO: 468 - protein, 9 aa, Borrelia burgdorferi
IVEKVSVIE

SEQ ID NO: 469 - protein, 9 aa, Borrelia burgdorferi
VEKVSVIEE

SEQ ID NO: 470 - protein, 9 aa, Borrelia burgdorferi
VSVIEEIAR

SEQ ID NO: 471 - protein, 9 aa, Borrelia burgdorferi
VIEEIARKT

SEQ ID NO: 472 - protein, 9 aa, Borrelia burgdorferi
IEEIARKTN

SEQ ID NO: 473 - protein, 9 aa, Borrelia burgdorferi
IARKTNLLA

SEQ ID NO: 474 - protein, 9 aa, Borrelia burgdorferi
LLALNAAIE

SEQ ID NO: 475 - protein, 9 aa, Borrelia burgdorferi
LALNAAIEA

SEQ ID NO: 476 - protein, 9 aa, Borrelia burgdorferi
LNAAIEAAR

SEQ ID NO: 477 - protein, 9 aa, Borrelia burgdorferi
FAVVASEIR

SEQ ID NO: 478 - protein, 9 aa, Borrelia burgdorferi
VVASEIRKL

SEQ ID NO: 479 - protein, 9 aa, Borrelia burgdorferi
VASEIRKLA

SEQ ID NO: 480 - protein, 9 aa, Borrelia burgdorferi
IRKLADLSK

SEQ ID NO: 481 - protein, 9 aa, Borrelia burgdorferi
LADLSKISA

SEQ ID NO: 482 - protein, 9 aa, Borrelia burgdorferi
LSKISALEI

SEQ ID NO: 483 - protein, 9 aa, Borrelia burgdorferi
ISALEIGEL

SEQ ID NO: 484 - protein, 9 aa, Borrelia burgdorferi
IGELVEDNS

SEQ ID NO: 485 - protein, 9 aa, Borrelia burgdorferi
LVEDNSKVA

SEQ ID NO: 486 - protein, 9 aa, Borrelia burgdorferi
VEDNSKVAT

SEQ ID NO: 487 - protein, 9 aa, Borrelia burgdorferi
KVATEAGVI

SEQ ID NO: 488 - protein, 9 aa, Borrelia burgdorferi
VATEAGVIF

SEQ ID NO: 489 - protein, 9 aa, Borrelia burgdorferi
VIFKEMLPE

SEQ ID NO: 490 - protein, 9 aa, Borrelia burgdorferi
FKEMLPEIE

SEQ ID NO: 491 - protein, 9 aa, Borrelia burgdorferi
IEETANLVK

SEQ ID NO: 492 - protein, 9 aa, Borrelia burgdorferi
LVKKISEGS

SEQ ID NO: 493 - protein, 9 aa, Borrelia burgdorferi
IAQFKMALD

SEQ ID NO: 494 - protein, 9 aa, Borrelia burgdorferi
FKMALDQVG

SEQ ID NO: 495 - protein, 9 aa, Borrelia burgdorferi
MALDQVGEV

SEQ ID NO: 496 - protein, 9 aa, Borrelia burgdorferi
LSSMSDKML

SEQ ID NO: 497 - protein, 9 aa, Borrelia burgdorferi
MLEKSKELR

SEQ ID NO: 498 - protein, 9 aa, Borrelia burgdorferi
VLFFKIKDS

SEQ ID NO: 499 - protein, 9 aa, Borrelia burgdorferi
FFKIKDSKI

SEQ ID NO: 500 - protein, 9 aa, Borrelia burgdorferi
LFFKIKDSK

SEQ ID NO: 501 - protein, 9 aa, Borrelia burgdorferi
FKIKDSKIE

SEQ ID NO: 502 - protein, 9 aa, Borrelia burgdorferi
FRLIDCPEN

SEQ ID NO: 503 - protein, 9 aa, Borrelia burgdorferi
FKDENQNLK

SEQ ID NO: 504 - protein, 9 aa, Borrelia burgdorferi
LKSNGISTS

SEQ ID NO: 505 - protein, 9 aa, Borrelia burgdorferi
YSLDIESES

SEQ ID NO: 506 - protein, 9 aa, Borrelia burgdorferi
IESESSVRT

SEQ ID NO: 507 - protein, 9 aa, Borrelia burgdorferi
VRTINKRVD

SEQ ID NO: 508 - protein, 9 aa, Borrelia burgdorferi
INKRVDPKK

SEQ ID NO: 509 - protein, 9 aa, Borrelia burgdorferi
VDPKKAIDI

SEQ ID NO: 510 - protein, 9 aa, Borrelia burgdorferi
IDIADKDLN

SEQ ID NO: 511 - protein, 9 aa, Borrelia burgdorferi
DIADKDLNF

SEQ ID NO: 512 - protein, 15 aa, Borrelia burgdorferi
TFSNAIFAQTIDDEN

SEQ ID NO: 513 - protein, 15 aa, Borrelia burgdorferi
FSNAIFAQTIDDENS

SEQ ID NO: 514 - protein, 15 aa, Borrelia burgdorferi
SNAIFAQTIDDENSK

SEQ ID NO: 515 - protein, 15 aa, Borrelia burgdorferi
NAIFAQTIDDENSKK

SEQ ID NO: 516 - protein, 15 aa, Borrelia burgdorferi
AIFAQTIDDENSKKR

SEQ ID NO: 517 - protein, 15 aa, Borrelia burgdorferi
FAQTIDDENSKKRDK

SEQ ID NO: 518 - protein, 15 aa, Borrelia burgdorferi
AQTIDDENSKKRDKL

SEQ ID NO: 519 - protein, 15 aa, Borrelia burgdorferi
SKKRDKLTLSQKSYL

SEQ ID NO: 520 - protein, 15 aa, Borrelia burgdorferi
KKRDKLTLSQKSYLR

SEQ ID NO: 521 - protein, 15 aa, Borrelia burgdorferi
KRDKLTLSQKSYLRE

SEQ ID NO: 522 - protein, 15 aa, Borrelia burgdorferi
RDKLTLSQKSYLREL

SEQ ID NO: 523 - protein, 15 aa, Borrelia burgdorferi
DKLTLSQKSYLRELE

SEQ ID NO: 524 - protein, 15 aa, Borrelia burgdorferi
LRELELSTDEDLKKW

SEQ ID NO: 525 - protein, 15 aa, Borrelia burgdorferi
RELELSTDEDLKKWA

SEQ ID NO: 526 - protein, 15 aa, Borrelia burgdorferi
ELELSTDEDLKKWAL

SEQ ID NO: 527 - protein, 15 aa, Borrelia burgdorferi
TDEDLKKWALKEGLK

SEQ ID NO: 528 - protein, 15 aa, Borrelia burgdorferi
DEDLKKWALKEGLKE

SEQ ID NO: 529 - protein, 15 aa, Borrelia burgdorferi
EDLKKWALKEGLKET

SEQ ID NO: 530 - protein, 15 aa, Borrelia burgdorferi
DLKKWALKEGLKETD

SEQ ID NO: 531 - protein, 15 aa, Borrelia burgdorferi
LKKWALKEGLKETDV

SEQ ID NO: 532 - protein, 15 aa, Borrelia burgdorferi
KKWALKEGLKETDVS

SEQ ID NO: 533 - protein, 15 aa, Borrelia burgdorferi
ALKEGLKETDVSKIR

SEQ ID NO: 534 - protein, 15 aa, Borrelia burgdorferi
LKEGLKETDVSKIRE

SEQ ID NO: 535 - protein, 15 aa, Borrelia burgdorferi
KEGLKETDVSKIREL

SEQ ID NO: 536 - protein, 15 aa, Borrelia burgdorferi
EGLKETDVSKIRELL

SEQ ID NO: 537 - protein, 15 aa, Borrelia burgdorferi
LKETDVSKIRELLLK

SEQ ID NO: 538 - protein, 15 aa, Borrelia burgdorferi
KETDVSKIRELLLKK

SEQ ID NO: 539 - protein, 15 aa, Borrelia burgdorferi
ETDVSKIRELLLKKF

SEQ ID NO: 540 - protein, 15 aa, Borrelia burgdorferi
TDVSKIRELLLKKFG

SEQ ID NO: 541 - protein, 15 aa, Borrelia burgdorferi
DVSKIRELLLKKFGI

SEQ ID NO: 542 - protein, 15 aa, Borrelia burgdorferi
VSKIRELLLKKFGID

SEQ ID NO: 543 - protein, 15 aa, Borrelia burgdorferi
SKIRELLLKKFGIDP

SEQ ID NO: 544 - protein, 15 aa, Borrelia burgdorferi
KIRELLLKKFGIDPE

SEQ ID NO: 545 - protein, 15 aa, Borrelia burgdorferi
ELLLKKFGIDPELFV

SEQ ID NO: 546 - protein, 15 aa, Borrelia burgdorferi
LLLKKFGIDPELFVK

SEQ ID NO: 547 - protein, 15 aa, Borrelia burgdorferi
LLKKFGIDPELFVKG

SEQ ID NO: 548 - protein, 15 aa, Borrelia burgdorferi
LKKFGIDPELFVKGK

SEQ ID NO: 549 - protein, 15 aa, Borrelia burgdorferi
KKFGIDPELFVKGKG

SEQ ID NO: 550 - protein, 15 aa, Borrelia burgdorferi
KFGIDPELFVKGKGL

SEQ ID NO: 551 - protein, 15 aa, Borrelia burgdorferi
GIDPELFVKGKGLAG

SEQ ID NO: 552 - protein, 15 aa, Borrelia burgdorferi
IDPELFVKGKGLAGS

SEQ ID NO: 553 - protein, 15 aa, Borrelia burgdorferi
DPELFVKGKGLAGSG

SEQ ID NO: 554 - protein, 15 aa, Borrelia burgdorferi
PELFVKGKGLAGSGR

SEQ ID NO: 555 - protein, 15 aa, Borrelia burgdorferi
ELFVKGKGLAGSGRY

SEQ ID NO: 556 - protein, 15 aa, Borrelia burgdorferi
VKGKGLAGSGRYKII

SEQ ID NO: 557 - protein, 15 aa, Borrelia burgdorferi
KGKGLAGSGRYKIII

SEQ ID NO: 558 - protein, 15 aa, Borrelia burgdorferi
GKGLAGSGRYKIIIE

SEQ ID NO: 559 - protein, 15 aa, Borrelia burgdorferi
KGLAGSGRYKIIIET

SEQ ID NO: 560 - protein, 15 aa, Borrelia burgdorferi
LAGSGRYKIIIETAD

SEQ ID NO: 561 - protein, 15 aa, Borrelia burgdorferi
AGSGRYKIIIETADN

SEQ ID NO: 562 - protein, 15 aa, Borrelia burgdorferi
GSGRYKIIIETADNL

SEQ ID NO: 563 - protein, 15 aa, Borrelia burgdorferi
SGRYKIIIETADNLE

SEQ ID NO: 564 - protein, 15 aa, Borrelia burgdorferi
GRYKIIIETADNLEN

SEQ ID NO: 565 - protein, 15 aa, Borrelia burgdorferi
RYKIIIETADNLENF

SEQ ID NO: 566 - protein, 15 aa, Borrelia burgdorferi
YKIIIETADNLENFT

SEQ ID NO: 567 - protein, 15 aa, Borrelia burgdorferi
KIIIETADNLENFTY

SEQ ID NO: 568 - protein, 15 aa, Borrelia burgdorferi
LENFTYGLTKDESII

SEQ ID NO: 569 - protein, 15 aa, Borrelia burgdorferi
ENFTYGLTKDESIIF

SEQ ID NO: 570 - protein, 15 aa, Borrelia burgdorferi
NFTYGLTKDESIIFE

SEQ ID NO: 571 - protein, 15 aa, Borrelia burgdorferi
FTYGLTKDESIIFEG

SEQ ID NO: 572 - protein, 15 aa, Borrelia burgdorferi
TYGLTKDESIIFEGR

SEQ ID NO: 573 - protein, 15 aa, Borrelia burgdorferi
YGLTKDESIIFEGRV

SEQ ID NO: 574 - protein, 15 aa, Borrelia burgdorferi
LTKDESIIFEGRVNI

SEQ ID NO: 575 - protein, 15 aa, Borrelia burgdorferi
TKDESIIFEGRVNIL

SEQ ID NO: 576 - protein, 15 aa, Borrelia burgdorferi
KDESIIFEGRVNILV

SEQ ID NO: 577 - protein, 15 aa, Borrelia burgdorferi
DESIIFEGRVNILVE

SEQ ID NO: 578 - protein, 15 aa, Borrelia burgdorferi
ESIIFEGRVNILVED

SEQ ID NO: 579 - protein, 15 aa, Borrelia burgdorferi
SIIFEGRVNILVEDI

SEQ ID NO: 580 - protein, 15 aa, Borrelia burgdorferi
EGRVNILVEDIKENK

SEQ ID NO: 581 - protein, 15 aa, Borrelia burgdorferi
GRVNILVEDIKENKK

SEQ ID NO: 582 - protein, 15 aa, Borrelia burgdorferi
RVNILVEDIKENKKH

SEQ ID NO: 583 - protein, 15 aa, Borrelia burgdorferi
VNILVEDIKENKKHN

SEQ ID NO: 584 - protein, 15 aa, Borrelia burgdorferi
NILVEDIKENKKHNI

SEQ ID NO: 585 - protein, 15 aa, Borrelia burgdorferi
ILVEDIKENKKHNIK

SEQ ID NO: 586 - protein, 15 aa, Borrelia burgdorferi
LVEDIKENKKHNIKG

SEQ ID NO: 587 - protein, 15 aa, Borrelia burgdorferi
VEDIKENKKHNIKGD

SEQ ID NO: 588 - protein, 15 aa, Borrelia burgdorferi
EDIKENKKHNIKGDR

SEQ ID NO: 589 - protein, 15 aa, Borrelia burgdorferi
DIKENKKHNIKGDRI

SEQ ID NO: 590 - protein, 15 aa, Borrelia burgdorferi
NKKHNIKGDRIVLNK

SEQ ID NO: 591 - protein, 15 aa, Borrelia burgdorferi
KKHNIKGDRIVLNKN

SEQ ID NO: 592 - protein, 15 aa, Borrelia burgdorferi
KHNIKGDRIVLNKNS

SEQ ID NO: 593 - protein, 15 aa, Borrelia burgdorferi
HNIKGDRIVLNKNSK

SEQ ID NO: 594 - protein, 15 aa, Borrelia burgdorferi
NIKGDRIVLNKNSKK

SEQ ID NO: 595 - protein, 15 aa, Borrelia burgdorferi
IKGDRIVLNKNSKKL

SEQ ID NO: 596 - protein, 15 aa, Borrelia burgdorferi
KGDRIVLNKNSKKLY

SEQ ID NO: 597 - protein, 15 aa, Borrelia burgdorferi
GDRIVLNKNSKKLYA

SEQ ID NO: 598 - protein, 15 aa, Borrelia burgdorferi
DRIVLNKNSKKLYAI

SEQ ID NO: 599 - protein, 15 aa, Borrelia burgdorferi
RIVLNKNSKKLYAIG

SEQ ID NO: 600 - protein, 15 aa, Borrelia burgdorferi
NKNSKKLYAIGNVEY

SEQ ID NO: 601 - protein, 15 aa, Borrelia burgdorferi
KNSKKLYAIGNVEYI

SEQ ID NO: 602 - protein, 15 aa, Borrelia burgdorferi
NSKKLYAIGNVEYIL

SEQ ID NO: 603 - protein, 15 aa, Borrelia burgdorferi
SKKLYAIGNVEYILD

SEQ ID NO: 604 - protein, 15 aa, Borrelia burgdorferi
KKLYAIGNVEYILDM

SEQ ID NO: 605 - protein, 15 aa, Borrelia burgdorferi
IGNVEYILDMDTNEK

SEQ ID NO: 606 - protein, 15 aa, Borrelia burgdorferi
GNVEYILDMDTNEKL

SEQ ID NO: 607 - protein, 15 aa, Borrelia burgdorferi
NVEYILDMDTNEKLY

SEQ ID NO: 608 - protein, 15 aa, Borrelia burgdorferi
VEYILDMDTNEKLYF

SEQ ID NO: 609 - protein, 15 aa, Borrelia burgdorferi
EYILDMDTNEKLYFY

SEQ ID NO: 610 - protein, 15 aa, Borrelia burgdorferi
YILDMDTNEKLYFYG

SEQ ID NO: 611 - protein, 15 aa, Borrelia burgdorferi
ILDMDTNEKLYFYGN

SEQ ID NO: 612 - protein, 15 aa, Borrelia burgdorferi
MDTNEKLYFYGNEFL

SEQ ID NO: 613 - protein, 15 aa, Borrelia burgdorferi
DTNEKLYFYGNEFLV

SEQ ID NO: 614 - protein, 15 aa, Borrelia burgdorferi
TNEKLYFYGNEFLVD

SEQ ID NO: 615 - protein, 15 aa, Borrelia burgdorferi
NEKLYFYGNEFLVDF

SEQ ID NO: 616 - protein, 15 aa, Borrelia burgdorferi
EKLYFYGNEFLVDFD

SEQ ID NO: 617 - protein, 15 aa, Borrelia burgdorferi
KLYFYGNEFLVDFDS

SEQ ID NO: 618 - protein, 15 aa, Borrelia burgdorferi
FYGNEFLVDFDSQNF

SEQ ID NO: 619 - protein, 15 aa, Borrelia burgdorferi
YGNEFLVDFDSQNFL

SEQ ID NO: 620 - protein, 15 aa, Borrelia burgdorferi
GNEFLVDFDSQNFLL

SEQ ID NO: 621 - protein, 15 aa, Borrelia burgdorferi
NEFLVDFDSQNFLLK

SEQ ID NO: 622 - protein, 15 aa, Borrelia burgdorferi
EFLVDFDSQNFLLKN

SEQ ID NO: 623 - protein, 15 aa, Borrelia burgdorferi
FDSQNFLLKNGILQK

SEQ ID NO: 624 - protein, 15 aa, Borrelia burgdorferi
DSQNFLLKNGILQKK

SEQ ID NO: 625 - protein, 15 aa, Borrelia burgdorferi
SQNFLLKNGILQKKM

SEQ ID NO: 626 - protein, 15 aa, Borrelia burgdorferi
QNFLLKNGILQKKMQ

SEQ ID NO: 627 - protein, 15 aa, Borrelia burgdorferi
MQKNQIDHILSFGGK

SEQ ID NO: 628 - protein, 15 aa, Borrelia burgdorferi
QKNQIDHILSFGGKV

SEQ ID NO: 629 - protein, 15 aa, Borrelia burgdorferi
KNQIDHILSFGGKVL

SEQ ID NO: 630 - protein, 15 aa, Borrelia burgdorferi
NQIDHILSFGGKVLK

SEQ ID NO: 631 - protein, 15 aa, Borrelia burgdorferi
QIDHILSFGGKVLKK

SEQ ID NO: 632 - protein, 15 aa, Borrelia burgdorferi
IDHILSFGGKVLKKI

SEQ ID NO: 633 - protein, 15 aa, Borrelia burgdorferi
DHILSFGGKVLKKID

SEQ ID NO: 634 - protein, 15 aa, Borrelia burgdorferi
HILSFGGKVLKKIDN

SEQ ID NO: 635 - protein, 15 aa, Borrelia burgdorferi
FGGKVLKKIDNDVTI

SEQ ID NO: 636 - protein, 15 aa, Borrelia burgdorferi
GGKVLKKIDNDVTIL

SEQ ID NO: 637 - protein, 15 aa, Borrelia burgdorferi
GKVLKKIDNDVTILE

SEQ ID NO: 638 - protein, 15 aa, Borrelia burgdorferi
KVLKKIDNDVTILEQ

SEQ ID NO: 639 - protein, 15 aa, Borrelia burgdorferi
VLKKIDNDVTILEQA

SEQ ID NO: 640 - protein, 15 aa, Borrelia burgdorferi
LKKIDNDVTILEQAF

SEQ ID NO: 641 - protein, 15 aa, Borrelia burgdorferi
KKIDNDVTILEQAFA

SEQ ID NO: 642 - protein, 15 aa, Borrelia burgdorferi
IDNDVTILEQAFATT

SEQ ID NO: 643 - protein, 15 aa, Borrelia burgdorferi
DNDVTILEQAFATTS

SEQ ID NO: 644 - protein, 15 aa, Borrelia burgdorferi
TILEQAFATTSKIPE

SEQ ID NO: 645 - protein, 15 aa, Borrelia burgdorferi
ILEQAFATTSKIPEP

SEQ ID NO: 646 - protein, 15 aa, Borrelia burgdorferi
LEQAFATTSKIPEPY

SEQ ID NO: 647 - protein, 15 aa, Borrelia burgdorferi
EQAFATTSKIPEPYY

SEQ ID NO: 648 - protein, 15 aa, Borrelia burgdorferi
QAFATTSKIPEPYYS

SEQ ID NO: 649 - protein, 15 aa, Borrelia burgdorferi
AFATTSKIPEPYYSI

SEQ ID NO: 650 - protein, 15 aa, Borrelia burgdorferi
TSKIPEPYYSIKASK

SEQ ID NO: 651 - protein, 15 aa, Borrelia burgdorferi
SKIPEPYYSIKASKI

SEQ ID NO: 652 - protein, 15 aa, Borrelia burgdorferi
KIPEPYYSIKASKIW

SEQ ID NO: 653 - protein, 15 aa, Borrelia burgdorferi
IPEPYYSIKASKIWA

SEQ ID NO: 654 - protein, 15 aa, Borrelia burgdorferi
PEPYYSIKASKIWAL

SEQ ID NO: 655 - protein, 15 aa, Borrelia burgdorferi
EPYYSIKASKIWALP

SEQ ID NO: 656 - protein, 15 aa, Borrelia burgdorferi
PYYSIKASKIWALPS

SEQ ID NO: 657 - protein, 15 aa, Borrelia burgdorferi
YYSIKASKIWALPSG

SEQ ID NO: 658 - protein, 15 aa, Borrelia burgdorferi
YSIKASKIWALPSGD

SEQ ID NO: 659 - protein, 15 aa, Borrelia burgdorferi
SIKASKIWALPSGDF

SEQ ID NO: 660 - protein, 15 aa, Borrelia burgdorferi
IKASKIWALPSGDFG

SEQ ID NO: 661 - protein, 15 aa, Borrelia burgdorferi
KASKIWALPSGDFGF

SEQ ID NO: 662 - protein, 15 aa, Borrelia burgdorferi
ASKIWALPSGDFGFL

SEQ ID NO: 663 - protein, 15 aa, Borrelia burgdorferi
SKIWALPSGDFGFLN

SEQ ID NO: 664 - protein, 15 aa, Borrelia burgdorferi
LPSGDFGFLNAIFYM

SEQ ID NO: 665 - protein, 15 aa, Borrelia burgdorferi
PSGDFGFLNAIFYMG

SEQ ID NO: 666 - protein, 15 aa, Borrelia burgdorferi
SGDFGFLNAIFYMGR

SEQ ID NO: 667 - protein, 15 aa, Borrelia burgdorferi
GDFGFLNAIFYMGRV

SEQ ID NO: 668 - protein, 15 aa, Borrelia burgdorferi
FGFLNAIFYMGRVPV

SEQ ID NO: 669 - protein, 15 aa, Borrelia burgdorferi
GFLNAIFYMGRVPVF

SEQ ID NO: 670 - protein, 15 aa, Borrelia burgdorferi
FLNAIFYMGRVPVFY

SEQ ID NO: 671 - protein, 15 aa, Borrelia burgdorferi
LNAIFYMGRVPVFYI

SEQ ID NO: 672 - protein, 15 aa, Borrelia burgdorferi
NAIFYMGRVPVFYIP

SEQ ID NO: 673 - protein, 15 aa, Borrelia burgdorferi
YIPFFFRPGDSLFFN

SEQ ID NO: 674 - protein, 15 aa, Borrelia burgdorferi
IPFFFRPGDSLFFNP

SEQ ID NO: 675 - protein, 15 aa, Borrelia burgdorferi
PFFFRPGDSLFFNPS

SEQ ID NO: 676 - protein, 15 aa, Borrelia burgdorferi
RPGDSLFFNPSLGLN

SEQ ID NO: 677 - protein, 15 aa, Borrelia burgdorferi
PGDSLFFNPSLGLNP

SEQ ID NO: 678 - protein, 15 aa, Borrelia burgdorferi
GDSLFFNPSLGLNPR

SEQ ID NO: 679 - protein, 15 aa, Borrelia burgdorferi
DSLFFNPSLGLNPRK

SEQ ID NO: 680 - protein, 15 aa, Borrelia burgdorferi
PSLGLNPRKGFAVFN

SEQ ID NO: 681 - protein, 15 aa, Borrelia burgdorferi
SLGLNPRKGFAVFNT

SEQ ID NO: 682 - protein, 15 aa, Borrelia burgdorferi
NPRKGFAVFNTVYLF

SEQ ID NO: 683 - protein, 15 aa, Borrelia burgdorferi
PRKGFAVFNTVYLFG

SEQ ID NO: 684 - protein, 15 aa, Borrelia burgdorferi
RKGFAVFNTVYLFGN

SEQ ID NO: 685 - protein, 15 aa, Borrelia burgdorferi
KGFAVFNTVYLFGNK

SEQ ID NO: 686 - protein, 15 aa, Borrelia burgdorferi
AVFNTVYLFGNKSSS

SEQ ID NO: 687 - protein, 15 aa, Borrelia burgdorferi
VFNTVYLFGNKSSSE

SEQ ID NO: 688 - protein, 15 aa, Borrelia burgdorferi
FNTVYLFGNKSSSED

SEQ ID NO: 689 - protein, 15 aa, Borrelia burgdorferi
NTVYLFGNKSSSEDS

SEQ ID NO: 690 - protein, 15 aa, Borrelia burgdorferi
TVYLFGNKSSSEDSS

SEQ ID NO: 691 - protein, 15 aa, Borrelia burgdorferi
VYLFGNKSSSEDSSF

SEQ ID NO: 692 - protein, 15 aa, Borrelia burgdorferi
EDSSFLDFDFNSVYN

SEQ ID NO: 693 - protein, 15 aa, Borrelia burgdorferi
DSSFLDFDFNSVYNS

SEQ ID NO: 694 - protein, 15 aa, Borrelia burgdorferi
SSFLDFDFNSVYNSG

SEQ ID NO: 695 - protein, 15 aa, Borrelia burgdorferi
SFLDFDFNSVYNSGK

SEQ ID NO: 696 - protein, 15 aa, Borrelia burgdorferi
FLDFDFNSVYNSGKK

SEQ ID NO: 697 - protein, 15 aa, Borrelia burgdorferi
DFDFNSVYNSGKKPY

SEQ ID NO: 698 - protein, 15 aa, Borrelia burgdorferi
DFNSVYNSGKKPYIR

SEQ ID NO: 699 - protein, 15 aa, Borrelia burgdorferi
FNSVYNSGKKPYIRN

SEQ ID NO: 700 - protein, 15 aa, Borrelia burgdorferi
NSVYNSGKKPYIRNG

SEQ ID NO: 701 - protein, 15 aa, Borrelia burgdorferi
KKPYIRNGYLTYFFA

SEQ ID NO: 702 - protein, 15 aa, Borrelia burgdorferi
IRNGYLTYFFAENLA

SEQ ID NO: 703 - protein, 15 aa, Borrelia burgdorferi
RNGYLTYFFAENLAP

SEQ ID NO: 704 - protein, 15 aa, Borrelia burgdorferi
NGYLTYFFAENLAPS

SEQ ID NO: 705 - protein, 15 aa, Borrelia burgdorferi
GYLTYFFAENLAPSV

SEQ ID NO: 706 - protein, 15 aa, Borrelia burgdorferi
YLTYFFAENLAPSVN

SEQ ID NO: 707 - protein, 15 aa, Borrelia burgdorferi
LTYFFAENLAPSVNK

SEQ ID NO: 708 - protein, 15 aa, Borrelia burgdorferi
TYFFAENLAPSVNKD

SEQ ID NO: 709 - protein, 15 aa, Borrelia burgdorferi
YFFAENLAPSVNKDY

SEQ ID NO: 710 - protein, 15 aa, Borrelia burgdorferi
LAPSVNKDYVKLIFD

SEQ ID NO: 711 - protein, 15 aa, Borrelia burgdorferi
APSVNKDYVKLIFDI

SEQ ID NO: 712 - protein, 15 aa, Borrelia burgdorferi
VNKDYVKLIFDIYAN

SEQ ID NO: 713 - protein, 15 aa, Borrelia burgdorferi
NKDYVKLIFDIYANL

SEQ ID NO: 714 - protein, 15 aa, Borrelia burgdorferi
KDYVKLIFDIYANLG

SEQ ID NO: 715 - protein, 15 aa, Borrelia burgdorferi
DYVKLIFDIYANLGF

SEQ ID NO: 716 - protein, 15 aa, Borrelia burgdorferi
YVKLIFDIYANLGFY

SEQ ID NO: 717 - protein, 15 aa, Borrelia burgdorferi
VKLIFDIYANLGFYS

SEQ ID NO: 718 - protein, 15 aa, Borrelia burgdorferi
KLIFDIYANLGFYSG

SEQ ID NO: 719 - protein, 15 aa, Borrelia burgdorferi
YANLGFYSGIDFNLG

SEQ ID NO: 720 - protein, 15 aa, Borrelia burgdorferi
GFYSGIDFNLGNTLG

SEQ ID NO: 721 - protein, 15 aa, Borrelia burgdorferi
FYSGIDFNLGNTLGH

SEQ ID NO: 722 - protein, 15 aa, Borrelia burgdorferi
YSGIDFNLGNTLGHF

SEQ ID NO: 723 - protein, 15 aa, Borrelia burgdorferi
SGIDFNLGNTLGHFK

SEQ ID NO: 724 - protein, 15 aa, Borrelia burgdorferi
NLGNTLGHFKTLEGN

SEQ ID NO: 725 - protein, 15 aa, Borrelia burgdorferi
LGNTLGHFKTLEGNF

SEQ ID NO: 726 - protein, 15 aa, Borrelia burgdorferi
GNTLGHFKTLEGNFG

SEQ ID NO: 727 - protein, 15 aa, Borrelia burgdorferi
NTLGHFKTLEGNFGL

SEQ ID NO: 728 - protein, 15 aa, Borrelia burgdorferi
TLGHFKTLEGNFGLG

SEQ ID NO: 729 - protein, 15 aa, Borrelia burgdorferi
LGHFKTLEGNFGLGF

SEQ ID NO: 730 - protein, 15 aa, Borrelia burgdorferi
GHFKTLEGNFGLGFT

SEQ ID NO: 731 - protein, 15 aa, Borrelia burgdorferi
NFGLGFTRNIYSYDG

SEQ ID NO: 732 - protein, 15 aa, Borrelia burgdorferi
LGFTRNIYSYDGGYY

SEQ ID NO: 733 - protein, 15 aa, Borrelia burgdorferi
GFTRNIYSYDGGYYP

SEQ ID NO: 734 - protein, 15 aa, Borrelia burgdorferi
FTRNIYSYDGGYYPF

SEQ ID NO: 735 - protein, 15 aa, Borrelia burgdorferi
TRNIYSYDGGYYPFD

SEQ ID NO: 736 - protein, 15 aa, Borrelia burgdorferi
RNIYSYDGGYYPFDN

SEQ ID NO: 737 - protein, 15 aa, Borrelia burgdorferi
YSYDGGYYPFDNRTL

SEQ ID NO: 738 - protein, 15 aa, Borrelia burgdorferi
SYDGGYYPFDNRTLK

SEQ ID NO: 739 - protein, 15 aa, Borrelia burgdorferi
YDGGYYPFDNRTLKQ

SEQ ID NO: 740 - protein, 15 aa, Borrelia burgdorferi
DGGYYPFDNRTLKQS

SEQ ID NO: 741 - protein, 15 aa, Borrelia burgdorferi
GGYYPFDNRTLKQSL

SEQ ID NO: 742 - protein, 15 aa, Borrelia burgdorferi
GYYPFDNRTLKQSLF

SEQ ID NO: 743 - protein, 15 aa, Borrelia burgdorferi
TLKQSLFSFSNLNKG

SEQ ID NO: 744 - protein, 15 aa, Borrelia burgdorferi
LKQSLFSFSNLNKGD

SEQ ID NO: 745 - protein, 15 aa, Borrelia burgdorferi
KQSLFSFSNLNKGDV

SEQ ID NO: 746 - protein, 15 aa, Borrelia burgdorferi
QSLFSFSNLNKGDVF

SEQ ID NO: 747 - protein, 15 aa, Borrelia burgdorferi
FGFEVPFRYLLKFKT

SEQ ID NO: 748 - protein, 15 aa, Borrelia burgdorferi
GFEVPFRYLLKFKTE

SEQ ID NO: 749 - protein, 15 aa, Borrelia burgdorferi
FEVPFRYLLKFKTEF

SEQ ID NO: 750 - protein, 15 aa, Borrelia burgdorferi
EVPFRYLLKFKTEFL

SEQ ID NO: 751 - protein, 15 aa, Borrelia burgdorferi
VPFRYLLKFKTEFLL

SEQ ID NO: 752 - protein, 15 aa, Borrelia burgdorferi
PFRYLLKFKTEFLLS

SEQ ID NO: 753 - protein, 15 aa, Borrelia burgdorferi
FRYLLKFKTEFLLSD

SEQ ID NO: 754 - protein, 15 aa, Borrelia burgdorferi
RYLLKFKTEFLLSDA

SEQ ID NO: 755 - protein, 15 aa, Borrelia burgdorferi
KFKTEFLLSDALFLV

SEQ ID NO: 756 - protein, 15 aa, Borrelia burgdorferi
FKTEFLLSDALFLVV

SEQ ID NO: 757 - protein, 15 aa, Borrelia burgdorferi
LSDALFLVVLEHYSD

SEQ ID NO: 758 - protein, 15 aa, Borrelia burgdorferi
SDALFLVVLEHYSDP

SEQ ID NO: 759 - protein, 15 aa, Borrelia burgdorferi
DALFLVVLEHYSDPY

SEQ ID NO: 760 - protein, 15 aa, Borrelia burgdorferi
ALFLVVLEHYSDPYV

SEQ ID NO: 761 - protein, 15 aa, Borrelia burgdorferi
LFLVVLEHYSDPYVN

SEQ ID NO: 762 - protein, 15 aa, Borrelia burgdorferi
FLVVLEHYSDPYVNI

SEQ ID NO: 763 - protein, 15 aa, Borrelia burgdorferi
LVVLEHYSDPYVNID

SEQ ID NO: 764 - protein, 15 aa, Borrelia burgdorferi
VVLEHYSDPYVNIDF

SEQ ID NO: 765 - protein, 15 aa, Borrelia burgdorferi
VLEHYSDPYVNIDFR

SEQ ID NO: 766 - protein, 15 aa, Borrelia burgdorferi
HYSDPYVNIDFRDRI

SEQ ID NO: 767 - protein, 15 aa, Borrelia burgdorferi
YSDPYVNIDFRDRIE

SEQ ID NO: 768 - protein, 15 aa, Borrelia burgdorferi
SDPYVNIDFRDRIES

SEQ ID NO: 769 - protein, 15 aa, Borrelia burgdorferi
DPYVNIDFRDRIESA

SEQ ID NO: 770 - protein, 15 aa, Borrelia burgdorferi
PYVNIDFRDRIESAT

SEQ ID NO: 771 - protein, 15 aa, Borrelia burgdorferi
YVNIDFRDRIESATF

SEQ ID NO: 772 - protein, 15 aa, Borrelia burgdorferi
VNIDFRDRIESATFF

SEQ ID NO: 773 - protein, 15 aa, Borrelia burgdorferi
SATFFSLLNLDKDSV

SEQ ID NO: 774 - protein, 15 aa, Borrelia burgdorferi
ATFFSLLNLDKDSVK

SEQ ID NO: 775 - protein, 15 aa, Borrelia burgdorferi
TFFSLLNLDKDSVKE

SEQ ID NO: 776 - protein, 15 aa, Borrelia burgdorferi
FFSLLNLDKDSVKEQ

SEQ ID NO: 777 - protein, 15 aa, Borrelia burgdorferi
FSLLNLDKDSVKEQT

SEQ ID NO: 778 - protein, 15 aa, Borrelia burgdorferi
SLLNLDKDSVKEQTS

SEQ ID NO: 779 - protein, 15 aa, Borrelia burgdorferi
DKDSVKEQTSISTFD

SEQ ID NO: 780 - protein, 15 aa, Borrelia burgdorferi
KDSVKEQTSISTFDW

SEQ ID NO: 781 - protein, 15 aa, Borrelia burgdorferi
FDWNLSSFYKRTFND

SEQ ID NO: 782 - protein, 15 aa, Borrelia burgdorferi
DWNLSSFYKRTFNDG

SEQ ID NO: 783 - protein, 15 aa, Borrelia burgdorferi
LSSFYKRTFNDGSIL

SEQ ID NO: 784 - protein, 15 aa, Borrelia burgdorferi
SSFYKRTFNDGSILD

SEQ ID NO: 785 - protein, 15 aa, Borrelia burgdorferi
FNDGSILDYKLNNLG

SEQ ID NO: 786 - protein, 15 aa, Borrelia burgdorferi
NDGSILDYKLNNLGL

SEQ ID NO: 787 - protein, 15 aa, Borrelia burgdorferi
DGSILDYKLNNLGLN

SEQ ID NO: 788 - protein, 15 aa, Borrelia burgdorferi
GSILDYKLNNLGLNF

SEQ ID NO: 789 - protein, 15 aa, Borrelia burgdorferi
LGLNFKLSGYENLYV

SEQ ID NO: 790 - protein, 15 aa, Borrelia burgdorferi
GLNFKLSGYENLYVK

SEQ ID NO: 791 - protein, 15 aa, Borrelia burgdorferi
LNFKLSGYENLYVKS

SEQ ID NO: 792 - protein, 15 aa, Borrelia burgdorferi
NFKLSGYENLYVKSP

SEQ ID NO: 793 - protein, 15 aa, Borrelia burgdorferi
FKLSGYENLYVKSPL

SEQ ID NO: 794 - protein, 15 aa, Borrelia burgdorferi
KLSGYENLYVKSPLE

SEQ ID NO: 795 - protein, 15 aa, Borrelia burgdorferi
YENLYVKSPLEKPKE

SEQ ID NO: 796 - protein, 15 aa, Borrelia burgdorferi
NDPTRKWFYLERIYA

SEQ ID NO: 797 - protein, 15 aa, Borrelia burgdorferi
DPTRKWFYLERIYAP

SEQ ID NO: 798 - protein, 15 aa, Borrelia burgdorferi
PTRKWFYLERIYAPY

SEQ ID NO: 799 - protein, 15 aa, Borrelia burgdorferi
TRKWFYLERIYAPYI

SEQ ID NO: 800 - protein, 15 aa, Borrelia burgdorferi
RKWFYLERIYAPYID

SEQ ID NO: 801 - protein, 15 aa, Borrelia burgdorferi
YLERIYAPYIDLNFQ

SEQ ID NO: 802 - protein, 15 aa, Borrelia burgdorferi
LERIYAPYIDLNFQK

SEQ ID NO: 803 - protein, 15 aa, Borrelia burgdorferi
ERIYAPYIDLNFQKD

SEQ ID NO: 804 - protein, 15 aa, Borrelia burgdorferi
RIYAPYIDLNFQKDL

SEQ ID NO: 805 - protein, 15 aa, Borrelia burgdorferi
APYIDLNFQKDLYNN

SEQ ID NO: 806 - protein, 15 aa, Borrelia burgdorferi
PYIDLNFQKDLYNNQ

SEQ ID NO: 807 - protein, 15 aa, Borrelia burgdorferi
YIDLNFQKDLYNNQW

SEQ ID NO: 808 - protein, 15 aa, Borrelia burgdorferi
IDLNFQKDLYNNQWT

SEQ ID NO: 809 - protein, 15 aa, Borrelia burgdorferi
LYNNQWTFSTDTKEM

SEQ ID NO: 810 - protein, 15 aa, Borrelia burgdorferi
YNNQWTFSTDTKEMI

SEQ ID NO: 811 - protein, 15 aa, Borrelia burgdorferi
NNQWTFSTDTKEMIM

SEQ ID NO: 812 - protein, 15 aa, Borrelia burgdorferi
NQWTFSTDTKEMIMR

SEQ ID NO: 813 - protein, 15 aa, Borrelia burgdorferi
QWTFSTDTKEMIMRP

SEQ ID NO: 814 - protein, 15 aa, Borrelia burgdorferi
WTFSTDTKEMIMRPE

SEQ ID NO: 815 - protein, 15 aa, Borrelia burgdorferi
TFSTDTKEMIMRPEI

SEQ ID NO: 816 - protein, 15 aa, Borrelia burgdorferi
DTKEMIMRPEIKNLE

SEQ ID NO: 817 - protein, 15 aa, Borrelia burgdorferi
TKEMIMRPEIKNLED

SEQ ID NO: 818 - protein, 15 aa, Borrelia burgdorferi
KEMIMRPEIKNLEDK

SEQ ID NO: 819 - protein, 15 aa, Borrelia burgdorferi
EMIMRPEIKNLEDKD

SEQ ID NO: 820 - protein, 15 aa, Borrelia burgdorferi
MIMRPEIKNLEDKDN

SEQ ID NO: 821 - protein, 15 aa, Borrelia burgdorferi
IMRPEIKNLEDKDND

SEQ ID NO: 822 - protein, 15 aa, Borrelia burgdorferi
MRPEIKNLEDKDNDK

SEQ ID NO: 823 - protein, 15 aa, Borrelia burgdorferi
RPEIKNLEDKDNDKK

SEQ ID NO: 824 - protein, 15 aa, Borrelia burgdorferi
PEIKNLEDKDNDKKS

SEQ ID NO: 825 - protein, 15 aa, Borrelia burgdorferi
KKTTKLTRDLYIPPE

SEQ ID NO: 826 - protein, 15 aa, Borrelia burgdorferi
IPPEPITLKNIDQSD

SEQ ID NO: 827 - protein, 15 aa, Borrelia burgdorferi
PPEPITLKNIDQSDS

SEQ ID NO: 828 - protein, 15 aa, Borrelia burgdorferi
PEPITLKNIDQSDSF

SEQ ID NO: 829 - protein, 15 aa, Borrelia burgdorferi
EPITLKNIDQSDSFF

SEQ ID NO: 830 - protein, 15 aa, Borrelia burgdorferi
ITLKNIDQSDSFFIR

SEQ ID NO: 831 - protein, 15 aa, Borrelia burgdorferi
TLKNIDQSDSFFIRF

SEQ ID NO: 832 - protein, 15 aa, Borrelia burgdorferi
LKNIDQSDSFFIRFG

SEQ ID NO: 833 - protein, 15 aa, Borrelia burgdorferi
KNIDQSDSFFIRFGI

SEQ ID NO: 834 - protein, 15 aa, Borrelia burgdorferi
SDSFFIRFGINPYLR

SEQ ID NO: 835 - protein, 15 aa, Borrelia burgdorferi
DSFFIRFGINPYLRN

SEQ ID NO: 836 - protein, 15 aa, Borrelia burgdorferi
SFFIRFGINPYLRNN

SEQ ID NO: 837 - protein, 15 aa, Borrelia burgdorferi
FFIRFGINPYLRNNV

SEQ ID NO: 838 - protein, 15 aa, Borrelia burgdorferi
FIRFGINPYLRNNVF

SEQ ID NO: 839 - protein, 15 aa, Borrelia burgdorferi
IRFGINPYLRNNVFF

SEQ ID NO: 840 - protein, 15 aa, Borrelia burgdorferi
RFGINPYLRNNVFFD

SEQ ID NO: 841 - protein, 15 aa, Borrelia burgdorferi
FGINPYLRNNVFFDN

SEQ ID NO: 842 - protein, 15 aa, Borrelia burgdorferi
GINPYLRNNVFFDNY

SEQ ID NO: 843 - protein, 15 aa, Borrelia burgdorferi
INPYLRNNVFFDNYG

SEQ ID NO: 844 - protein, 15 aa, Borrelia burgdorferi
NPYLRNNVFFDNYGI

SEQ ID NO: 845 - protein, 15 aa, Borrelia burgdorferi
YLRNNVFFDNYGITS

SEQ ID NO: 846 - protein, 15 aa, Borrelia burgdorferi
LRNNVFFDNYGITSP

SEQ ID NO: 847 - protein, 15 aa, Borrelia burgdorferi
RNNVFFDNYGITSPK

SEQ ID NO: 848 - protein, 15 aa, Borrelia burgdorferi
NNVFFDNYGITSPKD

SEQ ID NO: 849 - protein, 15 aa, Borrelia burgdorferi
VFFDNYGITSPKDFN

SEQ ID NO: 850 - protein, 15 aa, Borrelia burgdorferi
FFDNYGITSPKDFNY

SEQ ID NO: 851 - protein, 15 aa, Borrelia burgdorferi
FDNYGITSPKDFNYQ

SEQ ID NO: 852 - protein, 15 aa, Borrelia burgdorferi
DNYGITSPKDFNYQI

SEQ ID NO: 853 - protein, 15 aa, Borrelia burgdorferi
ITSPKDFNYQIKNYL

SEQ ID NO: 854 - protein, 15 aa, Borrelia burgdorferi
TSPKDFNYQIKNYLF

SEQ ID NO: 855 - protein, 15 aa, Borrelia burgdorferi
SPKDFNYQIKNYLFD

SEQ ID NO: 856 - protein, 15 aa, Borrelia burgdorferi
PKDFNYQIKNYLFDI

SEQ ID NO: 857 - protein, 15 aa, Borrelia burgdorferi
KDFNYQIKNYLFDIK

SEQ ID NO: 858 - protein, 15 aa, Borrelia burgdorferi
DFNYQIKNYLFDIKN

SEQ ID NO: 859 - protein, 15 aa, Borrelia burgdorferi
NYQIKNYLFDIKNKT

SEQ ID NO: 860 - protein, 15 aa, Borrelia burgdorferi
YQIKNYLFDIKNKTD

SEQ ID NO: 861 - protein, 15 aa, Borrelia burgdorferi
QIKNYLFDIKNKTDI

SEQ ID NO: 862 - protein, 15 aa, Borrelia burgdorferi
IKNYLFDIKNKTDIK

SEQ ID NO: 863 - protein, 15 aa, Borrelia burgdorferi
KNYLFDIKNKTDIKI

SEQ ID NO: 864 - protein, 15 aa, Borrelia burgdorferi
NYLFDIKNKTDIKIH

SEQ ID NO: 865 - protein, 15 aa, Borrelia burgdorferi
YLFDIKNKTDIKIHA

SEQ ID NO: 866 - protein, 15 aa, Borrelia burgdorferi
LFDIKNKTDIKIHAD

SEQ ID NO: 867 - protein, 15 aa, Borrelia burgdorferi
FDIKNKTDIKIHADF

SEQ ID NO: 868 - protein, 15 aa, Borrelia burgdorferi
DIKNKTDIKIHADFY

SEQ ID NO: 869 - protein, 15 aa, Borrelia burgdorferi
KNKTDIKIHADFYNR

SEQ ID NO: 870 - protein, 15 aa, Borrelia burgdorferi
NKTDIKIHADFYNRL

SEQ ID NO: 871 - protein, 15 aa, Borrelia burgdorferi
KTDIKIHADFYNRLI

SEQ ID NO: 872 - protein, 15 aa, Borrelia burgdorferi
TDIKIHADFYNRLIT

SEQ ID NO: 873 - protein, 15 aa, Borrelia burgdorferi
DIKIHADFYNRLITF

SEQ ID NO: 874 - protein, 15 aa, Borrelia burgdorferi
IHADFYNRLITFENL

SEQ ID NO: 875 - protein, 15 aa, Borrelia burgdorferi
HADFYNRLITFENLL

SEQ ID NO: 876 - protein, 15 aa, Borrelia burgdorferi
ADFYNRLITFENLLY

SEQ ID NO: 877 - protein, 15 aa, Borrelia burgdorferi
FYNRLITFENLLYLN

SEQ ID NO: 878 - protein, 15 aa, Borrelia burgdorferi
YNRLITFENLLYLNT

SEQ ID NO: 879 - protein, 15 aa, Borrelia burgdorferi
NRLITFENLLYLNTI

SEQ ID NO: 880 - protein, 15 aa, Borrelia burgdorferi
LITFENLLYLNTIEY

SEQ ID NO: 881 - protein, 15 aa, Borrelia burgdorferi
ITFENLLYLNTIEYS

SEQ ID NO: 882 - protein, 15 aa, Borrelia burgdorferi
TFENLLYLNTIEYSP

SEQ ID NO: 883 - protein, 15 aa, Borrelia burgdorferi
FENLLYLNTIEYSPL

SEQ ID NO: 884 - protein, 15 aa, Borrelia burgdorferi
ENLLYLNTIEYSPLN

SEQ ID NO: 885 - protein, 15 aa, Borrelia burgdorferi
NLLYLNTIEYSPLNK

SEQ ID NO: 886 - protein, 15 aa, Borrelia burgdorferi
YLNTIEYSPLNKDFK

SEQ ID NO: 887 - protein, 15 aa, Borrelia burgdorferi
LNTIEYSPLNKDFKV

SEQ ID NO: 888 - protein, 15 aa, Borrelia burgdorferi
NTIEYSPLNKDFKVE

SEQ ID NO: 889 - protein, 15 aa, Borrelia burgdorferi
TIEYSPLNKDFKVED

SEQ ID NO: 890 - protein, 15 aa, Borrelia burgdorferi
IEYSPLNKDFKVEDK

SEQ ID NO: 891 - protein, 15 aa, Borrelia burgdorferi
EYSPLNKDFKVEDKD

SEQ ID NO: 892 - protein, 15 aa, Borrelia burgdorferi
YSPLNKDFKVEDKDK

SEQ ID NO: 893 - protein, 15 aa, Borrelia burgdorferi
SPLNKDFKVEDKDKK

SEQ ID NO: 894 - protein, 15 aa, Borrelia burgdorferi
PLNKDFKVEDKDKKS

SEQ ID NO: 895 - protein, 15 aa, Borrelia burgdorferi
LNKDFKVEDKDKKSE

SEQ ID NO: 896 - protein, 15 aa, Borrelia burgdorferi
NKDFKVEDKDKKSEH

SEQ ID NO: 897 - protein, 15 aa, Borrelia burgdorferi
KDFKVEDKDKKSEHS

SEQ ID NO: 898 - protein, 15 aa, Borrelia burgdorferi
KSEHSIINQINLNLL

SEQ ID NO: 899 - protein, 15 aa, Borrelia burgdorferi
SEHSIINQINLNLLP

SEQ ID NO: 900 - protein, 15 aa, Borrelia burgdorferi
EHSIINQINLNLLPF

SEQ ID NO: 901 - protein, 15 aa, Borrelia burgdorferi
IINQINLNLLPFIRY

SEQ ID NO: 902 - protein, 15 aa, Borrelia burgdorferi
INQINLNLLPFIRYP

SEQ ID NO: 903 - protein, 15 aa, Borrelia burgdorferi
NQINLNLLPFIRYPL

SEQ ID NO: 904 - protein, 15 aa, Borrelia burgdorferi
QINLNLLPFIRYPLF

SEQ ID NO: 905 - protein, 15 aa, Borrelia burgdorferi
INLNLLPFIRYPLFS

SEQ ID NO: 906 - protein, 15 aa, Borrelia burgdorferi
NLNLLPFIRYPLFSR

SEQ ID NO: 907 - protein, 15 aa, Borrelia burgdorferi
LNLLPFIRYPLFSRS

SEQ ID NO: 908 - protein, 15 aa, Borrelia burgdorferi
RYPLFSRSTLKFENK

SEQ ID NO: 909 - protein, 15 aa, Borrelia burgdorferi
LFSRSTLKFENKATL

SEQ ID NO: 910 - protein, 15 aa, Borrelia burgdorferi
FSRSTLKFENKATLY

SEQ ID NO: 911 - protein, 15 aa, Borrelia burgdorferi
SRSTLKFENKATLYS

SEQ ID NO: 912 - protein, 15 aa, Borrelia burgdorferi
RSTLKFENKATLYSF

SEQ ID NO: 913 - protein, 15 aa, Borrelia burgdorferi
STLKFENKATLYSFN

SEQ ID NO: 914 - protein, 15 aa, Borrelia burgdorferi
TLKFENKATLYSFNK

SEQ ID NO: 915 - protein, 15 aa, Borrelia burgdorferi
LKFENKATLYSFNKK

SEQ ID NO: 916 - protein, 15 aa, Borrelia burgdorferi
ENKATLYSFNKKYDS

SEQ ID NO: 917 - protein, 15 aa, Borrelia burgdorferi
NKATLYSFNKKYDSD

SEQ ID NO: 918 - protein, 15 aa, Borrelia burgdorferi
KATLYSFNKKYDSDV

SEQ ID NO: 919 - protein, 15 aa, Borrelia burgdorferi
ATLYSFNKKYDSDVK

SEQ ID NO: 920 - protein, 15 aa, Borrelia burgdorferi
TLYSFNKKYDSDVKS

SEQ ID NO: 921 - protein, 15 aa, Borrelia burgdorferi
YSFNKKYDSDVKSLV

SEQ ID NO: 922 - protein, 15 aa, Borrelia burgdorferi
SFNKKYDSDVKSLVN

SEQ ID NO: 923 - protein, 15 aa, Borrelia burgdorferi
FNKKYDSDVKSLVNK

SEQ ID NO: 924 - protein, 15 aa, Borrelia burgdorferi
NKKYDSDVKSLVNKN

SEQ ID NO: 925 - protein, 15 aa, Borrelia burgdorferi
KKYDSDVKSLVNKNS

SEQ ID NO: 926 - protein, 15 aa, Borrelia burgdorferi
KYDSDVKSLVNKNSS

SEQ ID NO: 927 - protein, 15 aa, Borrelia burgdorferi
YDSDVKSLVNKNSSI

SEQ ID NO: 928 - protein, 15 aa, Borrelia burgdorferi
DSDVKSLVNKNSSIF

SEQ ID NO: 929 - protein, 15 aa, Borrelia burgdorferi
SDVKSLVNKNSSIFL

SEQ ID NO: 930 - protein, 15 aa, Borrelia burgdorferi
DVKSLVNKNSSIFLS

SEQ ID NO: 931 - protein, 15 aa, Borrelia burgdorferi
VKSLVNKNSSIFLSD

SEQ ID NO: 932 - protein, 15 aa, Borrelia burgdorferi
KSLVNKNSSIFLSDP

SEQ ID NO: 933 - protein, 15 aa, Borrelia burgdorferi
NKNSSIFLSDPETFY

SEQ ID NO: 934 - protein, 15 aa, Borrelia burgdorferi
KNSSIFLSDPETFYQ

SEQ ID NO: 935 - protein, 15 aa, Borrelia burgdorferi
NSSIFLSDPETFYQS

SEQ ID NO: 936 - protein, 15 aa, Borrelia burgdorferi
SSIFLSDPETFYQSL

SEQ ID NO: 937 - protein, 15 aa, Borrelia burgdorferi
SIFLSDPETFYQSLT

SEQ ID NO: 938 - protein, 15 aa, Borrelia burgdorferi
LSDPETFYQSLTASL

SEQ ID NO: 939 - protein, 15 aa, Borrelia burgdorferi
SDPETFYQSLTASLI

SEQ ID NO: 940 - protein, 15 aa, Borrelia burgdorferi
DPETFYQSLTASLIY

SEQ ID NO: 941 - protein, 15 aa, Borrelia burgdorferi
PETFYQSLTASLIYD

SEQ ID NO: 942 - protein, 15 aa, Borrelia burgdorferi
ETFYQSLTASLIYDY

SEQ ID NO: 943 - protein, 15 aa, Borrelia burgdorferi
TFYQSLTASLIYDYD

SEQ ID NO: 944 - protein, 15 aa, Borrelia burgdorferi
FYQSLTASLIYDYDY

SEQ ID NO: 945 - protein, 15 aa, Borrelia burgdorferi
SLIYDYDYFTTELSG

SEQ ID NO: 946 - protein, 15 aa, Borrelia burgdorferi
LIYDYDYFTTELSGE

SEQ ID NO: 947 - protein, 15 aa, Borrelia burgdorferi
IYDYDYFTTELSGEL

SEQ ID NO: 948 - protein, 15 aa, Borrelia burgdorferi
YDYDYFTTELSGELK

SEQ ID NO: 949 - protein, 15 aa, Borrelia burgdorferi
DYDYFTTELSGELKN

SEQ ID NO: 950 - protein, 15 aa, Borrelia burgdorferi
YDYFTTELSGELKNS

SEQ ID NO: 951 - protein, 15 aa, Borrelia burgdorferi
FTTELSGELKNSFED

SEQ ID NO: 952 - protein, 15 aa, Borrelia burgdorferi
TTELSGELKNSFEDI

SEQ ID NO: 953 - protein, 15 aa, Borrelia burgdorferi
ELKNSFEDIKASSEL

SEQ ID NO: 954 - protein, 15 aa, Borrelia burgdorferi
LKNSFEDIKASSELK

SEQ ID NO: 955 - protein, 15 aa, Borrelia burgdorferi
KNSFEDIKASSELKL

SEQ ID NO: 956 - protein, 15 aa, Borrelia burgdorferi
NSFEDIKASSELKLS

SEQ ID NO: 957 - protein, 15 aa, Borrelia burgdorferi
SFEDIKASSELKLSL

SEQ ID NO: 958 - protein, 15 aa, Borrelia burgdorferi
FEDIKASSELKLSLD

SEQ ID NO: 959 - protein, 15 aa, Borrelia burgdorferi
EDIKASSELKLSLDF

SEQ ID NO: 960 - protein, 15 aa, Borrelia burgdorferi
ELKLSLDFPYLLQEA

SEQ ID NO: 961 - protein, 15 aa, Borrelia burgdorferi
SLDFPYLLQEAGIGI

SEQ ID NO: 962 - protein, 15 aa, Borrelia burgdorferi
LDFPYLLQEAGIGIK

SEQ ID NO: 963 - protein, 15 aa, Borrelia burgdorferi
DFPYLLQEAGIGIKY

SEQ ID NO: 964 - protein, 15 aa, Borrelia burgdorferi
FPYLLQEAGIGIKYY

SEQ ID NO: 965 - protein, 15 aa, Borrelia burgdorferi
PYLLQEAGIGIKYYK

SEQ ID NO: 966 - protein, 15 aa, Borrelia burgdorferi
QEAGIGIKYYKKFKE

SEQ ID NO: 967 - protein, 15 aa, Borrelia burgdorferi
EAGIGIKYYKKFKED

SEQ ID NO: 968 - protein, 15 aa, Borrelia burgdorferi
AGIGIKYYKKFKEDA

SEQ ID NO: 969 - protein, 15 aa, Borrelia burgdorferi
GIGIKYYKKFKEDAM

SEQ ID NO: 970 - protein, 15 aa, Borrelia burgdorferi
IGIKYYKKFKEDAMK

SEQ ID NO: 971 - protein, 15 aa, Borrelia burgdorferi
GIKYYKKFKEDAMKN

SEQ ID NO: 972 - protein, 15 aa, Borrelia burgdorferi
IKYYKKFKEDAMKNS

SEQ ID NO: 973 - protein, 15 aa, Borrelia burgdorferi
MKNSGISAVQSPLEP

SEQ ID NO: 974 - protein, 15 aa, Borrelia burgdorferi
KNSGISAVQSPLEPQ

SEQ ID NO: 975 - protein, 15 aa, Borrelia burgdorferi
NSGISAVQSPLEPQK

SEQ ID NO: 976 - protein, 15 aa, Borrelia burgdorferi
SGISAVQSPLEPQKP

SEQ ID NO: 977 - protein, 15 aa, Borrelia burgdorferi
PSSPYKNLEMSPALY

SEQ ID NO: 978 - protein, 15 aa, Borrelia burgdorferi
SSPYKNLEMSPALYY

SEQ ID NO: 979 - protein, 15 aa, Borrelia burgdorferi
SPYKNLEMSPALYYK

SEQ ID NO: 980 - protein, 15 aa, Borrelia burgdorferi
PYKNLEMSPALYYKI

SEQ ID NO: 981 - protein, 15 aa, Borrelia burgdorferi
YKNLEMSPALYYKIE

SEQ ID NO: 982 - protein, 15 aa, Borrelia burgdorferi
KNLEMSPALYYKIEP

SEQ ID NO: 983 - protein, 15 aa, Borrelia burgdorferi
MSPALYYKIEPRYLD

SEQ ID NO: 984 - protein, 15 aa, Borrelia burgdorferi
SPALYYKIEPRYLDY

SEQ ID NO: 985 - protein, 15 aa, Borrelia burgdorferi
PALYYKIEPRYLDYF

SEQ ID NO: 986 - protein, 15 aa, Borrelia burgdorferi
ALYYKIEPRYLDYFK

SEQ ID NO: 987 - protein, 15 aa, Borrelia burgdorferi
LYYKIEPRYLDYFKF

SEQ ID NO: 988 - protein, 15 aa, Borrelia burgdorferi
YYKIEPRYLDYFKFS

SEQ ID NO: 989 - protein, 15 aa, Borrelia burgdorferi
FKFSFLVAYDPLINR

SEQ ID NO: 990 - protein, 15 aa, Borrelia burgdorferi
KFSFLVAYDPLINRV

SEQ ID NO: 991 - protein, 15 aa, Borrelia burgdorferi
FSFLVAYDPLINRVS

SEQ ID NO: 992 - protein, 15 aa, Borrelia burgdorferi
SFLVAYDPLINRVSE

SEQ ID NO: 993 - protein, 15 aa, Borrelia burgdorferi
FLVAYDPLINRVSEL

SEQ ID NO: 994 - protein, 15 aa, Borrelia burgdorferi
LVAYDPLINRVSELS

SEQ ID NO: 995 - protein, 15 aa, Borrelia burgdorferi
AYDPLINRVSELSFK

SEQ ID NO: 996 - protein, 15 aa, Borrelia burgdorferi
YDPLINRVSELSFKL

SEQ ID NO: 997 - protein, 15 aa, Borrelia burgdorferi
DPLINRVSELSFKLN

SEQ ID NO: 998 - protein, 15 aa, Borrelia burgdorferi
PLINRVSELSFKLNV

SEQ ID NO: 999 - protein, 15 aa, Borrelia burgdorferi
LNVFDFQFLFAMKDE

SEQ ID NO: 1000 - protein, 15 aa, Borrelia burgdorferi
NVFDFQFLFAMKDEF

SEQ ID NO: 1001 - protein, 15 aa, Borrelia burgdorferi
VFDFQFLFAMKDEFE

SEQ ID NO: 1002 - protein, 15 aa, Borrelia burgdorferi
FDFQFLFAMKDEFEY

SEQ ID NO: 1003 - protein, 15 aa, Borrelia burgdorferi
DFQFLFAMKDEFEYN

SEQ ID NO: 1004 - protein, 15 aa, Borrelia burgdorferi
FQFLFAMKDEFEYNY

SEQ ID NO: 1005 - protein, 15 aa, Borrelia burgdorferi
QFLFAMKDEFEYNYD

SEQ ID NO: 1006 - protein, 15 aa, Borrelia burgdorferi
MKDEFEYNYDPLKGD

SEQ ID NO: 1007 - protein, 15 aa, Borrelia burgdorferi
KDEFEYNYDPLKGDF

SEQ ID NO: 1008 - protein, 15 aa, Borrelia burgdorferi
DEFEYNYDPLKGDFS

SEQ ID NO: 1009 - protein, 15 aa, Borrelia burgdorferi
EFEYNYDPLKGDFSK

SEQ ID NO: 1010 - protein, 15 aa, Borrelia burgdorferi
FEYNYDPLKGDFSKI

SEQ ID NO: 1011 - protein, 15 aa, Borrelia burgdorferi
EYNYDPLKGDFSKIG

SEQ ID NO: 1012 - protein, 15 aa, Borrelia burgdorferi
YNYDPLKGDFSKIGT

SEQ ID NO: 1013 - protein, 15 aa, Borrelia burgdorferi
TTTKLVPYSLDSSYK

SEQ ID NO: 1014 - protein, 15 aa, Borrelia burgdorferi
TTKLVPYSLDSSYKK

SEQ ID NO: 1015 - protein, 15 aa, Borrelia burgdorferi
TKLVPYSLDSSYKKE

SEQ ID NO: 1016 - protein, 15 aa, Borrelia burgdorferi
KLVPYSLDSSYKKEL

SEQ ID NO: 1017 - protein, 15 aa, Borrelia burgdorferi
LVPYSLDSSYKKELY

SEQ ID NO: 1018 - protein, 15 aa, Borrelia burgdorferi
VPYSLDSSYKKELYV

SEQ ID NO: 1019 - protein, 15 aa, Borrelia burgdorferi
KKELYVLTFFDNKLS

SEQ ID NO: 1020 - protein, 15 aa, Borrelia burgdorferi
KELYVLTFFDNKLSF

SEQ ID NO: 1021 - protein, 15 aa, Borrelia burgdorferi
ELYVLTFFDNKLSFT

SEQ ID NO: 1022 - protein, 15 aa, Borrelia burgdorferi
LYVLTFFDNKLSFTL

SEQ ID NO: 1023 - protein, 15 aa, Borrelia burgdorferi
YVLTFFDNKLSFTLG

SEQ ID NO: 1024 - protein, 15 aa, Borrelia burgdorferi
TFFDNKLSFTLGVDV

SEQ ID NO: 1025 - protein, 15 aa, Borrelia burgdorferi
FFDNKLSFTLGVDVG

SEQ ID NO: 1026 - protein, 15 aa, Borrelia burgdorferi
FDNKLSFTLGVDVGW

SEQ ID NO: 1027 - protein, 15 aa, Borrelia burgdorferi
DNKLSFTLGVDVGWK

SEQ ID NO: 1028 - protein, 15 aa, Borrelia burgdorferi
NKLSFTLGVDVGWKI

SEQ ID NO: 1029 - protein, 15 aa, Borrelia burgdorferi
GWKINLQKFTDNELR

SEQ ID NO: 1030 - protein, 15 aa, Borrelia burgdorferi
WKINLQKFTDNELRS

SEQ ID NO: 1031 - protein, 15 aa, Borrelia burgdorferi
KINLQKFTDNELRSA

SEQ ID NO: 1032 - protein, 15 aa, Borrelia burgdorferi
INLQKFTDNELRSAL

SEQ ID NO: 1033 - protein, 15 aa, Borrelia burgdorferi
FTDNELRSALTLKLK

SEQ ID NO: 1034 - protein, 15 aa, Borrelia burgdorferi
TDNELRSALTLKLKY

SEQ ID NO: 1035 - protein, 15 aa, Borrelia burgdorferi
DNELRSALTLKLKYT

SEQ ID NO: 1036 - protein, 15 aa, Borrelia burgdorferi
KYTEFLEIYFSTLSI

SEQ ID NO: 1037 - protein, 15 aa, Borrelia burgdorferi
YTEFLEIYFSTLSIN

SEQ ID NO: 1038 - protein, 15 aa, Borrelia burgdorferi
TEFLEIYFSTLSINT

SEQ ID NO: 1039 - protein, 15 aa, Borrelia burgdorferi
EFLEIYFSTLSINTK

SEQ ID NO: 1040 - protein, 15 aa, Borrelia burgdorferi
FLEIYFSTLSINTKT

SEQ ID NO: 1041 - protein, 15 aa, Borrelia burgdorferi
LEIYFSTLSINTKTF

SEQ ID NO: 1042 - protein, 15 aa, Borrelia burgdorferi
EIYFSTLSINTKTFK

SEQ ID NO: 1043 - protein, 15 aa, Borrelia burgdorferi
SINTKTFKYFKGYMD

SEQ ID NO: 1044 - protein, 15 aa, Borrelia burgdorferi
INTKTFKYFKGYMDQ

SEQ ID NO: 1045 - protein, 15 aa, Borrelia burgdorferi
NTKTFKYFKGYMDQI

SEQ ID NO: 1046 - protein, 15 aa, Borrelia burgdorferi
TKTFKYFKGYMDQIG

SEQ ID NO: 1047 - protein, 15 aa, Borrelia burgdorferi
KTFKYFKGYMDQIGL

SEQ ID NO: 1048 - protein, 15 aa, Borrelia burgdorferi
TFKYFKGYMDQIGLE

SEQ ID NO: 1049 - protein, 15 aa, Borrelia burgdorferi
FKYFKGYMDQIGLEP

SEQ ID NO: 1050 - protein, 15 aa, Borrelia burgdorferi
KYFKGYMDQIGLEPV

SEQ ID NO: 1051 - protein, 15 aa, Borrelia burgdorferi
MDQIGLEPVNVFADL

SEQ ID NO: 1052 - protein, 15 aa, Borrelia burgdorferi
DQIGLEPVNVFADLL

SEQ ID NO: 1053 - protein, 15 aa, Borrelia burgdorferi
QIGLEPVNVFADLLK

SEQ ID NO: 1054 - protein, 15 aa, Borrelia burgdorferi
LEPVNVFADLLKSFN

SEQ ID NO: 1055 - protein, 15 aa, Borrelia burgdorferi
DLLKSFNFFNSQDRK

SEQ ID NO: 1056 - protein, 15 aa, Borrelia burgdorferi
LLKSFNFFNSQDRKD

SEQ ID NO: 1057 - protein, 15 aa, Borrelia burgdorferi
LKSFNFFNSQDRKDS

SEQ ID NO: 1058 - protein, 15 aa, Borrelia burgdorferi
KSFNFFNSQDRKDSL

SEQ ID NO: 1059 - protein, 15 aa, Borrelia burgdorferi
SFNFFNSQDRKDSLF

SEQ ID NO: 1060 - protein, 15 aa, Borrelia burgdorferi
FNFFNSQDRKDSLFK

SEQ ID NO: 1061 - protein, 15 aa, Borrelia burgdorferi
DRKDSLFKIKKFSSG

SEQ ID NO: 1062 - protein, 15 aa, Borrelia burgdorferi
RKDSLFKIKKFSSGF

SEQ ID NO: 1063 - protein, 15 aa, Borrelia burgdorferi
KDSLFKIKKFSSGFK

SEQ ID NO: 1064 - protein, 15 aa, Borrelia burgdorferi
DSLFKIKKFSSGFKF

SEQ ID NO: 1065 - protein, 15 aa, Borrelia burgdorferi
FNFYDWKFVGEYNLE

SEQ ID NO: 1066 - protein, 15 aa, Borrelia burgdorferi
NFYDWKFVGEYNLEP

SEQ ID NO: 1067 - protein, 15 aa, Borrelia burgdorferi
FYDWKFVGEYNLEPD

SEQ ID NO: 1068 - protein, 15 aa, Borrelia burgdorferi
YDWKFVGEYNLEPDL

SEQ ID NO: 1069 - protein, 15 aa, Borrelia burgdorferi
DWKFVGEYNLEPDLL

SEQ ID NO: 1070 - protein, 15 aa, Borrelia burgdorferi
KFVGEYNLEPDLLIG

SEQ ID NO: 1071 - protein, 15 aa, Borrelia burgdorferi
FVGEYNLEPDLLIGS

SEQ ID NO: 1072 - protein, 15 aa, Borrelia burgdorferi
VGEYNLEPDLLIGSD

SEQ ID NO: 1073 - protein, 15 aa, Borrelia burgdorferi
GEYNLEPDLLIGSDG

SEQ ID NO: 1074 - protein, 15 aa, Borrelia burgdorferi
GSDGIYSPIWRNNFT

SEQ ID NO: 1075 - protein, 15 aa, Borrelia burgdorferi
SDGIYSPIWRNNFTI

SEQ ID NO: 1076 - protein, 15 aa, Borrelia burgdorferi
DGIYSPIWRNNFTIY

SEQ ID NO: 1077 - protein, 15 aa, Borrelia burgdorferi
IYSPIWRNNFTIYIS

SEQ ID NO: 1078 - protein, 15 aa, Borrelia burgdorferi
YSPIWRNNFTIYISW

SEQ ID NO: 1079 - protein, 15 aa, Borrelia burgdorferi
SPIWRNNFTIYISWN

SEQ ID NO: 1080 - protein, 15 aa, Borrelia burgdorferi
IWRNNFTIYISWNFF

SEQ ID NO: 1081 - protein, 15 aa, Borrelia burgdorferi
WRNNFTIYISWNFFA

SEQ ID NO: 1082 - protein, 15 aa, Borrelia burgdorferi
RNNFTIYISWNFFAP

SEQ ID NO: 1083 - protein, 15 aa, Borrelia burgdorferi
NNFTIYISWNFFAPI

SEQ ID NO: 1084 - protein, 15 aa, Borrelia burgdorferi
NFTIYISWNFFAPIK

SEQ ID NO: 1085 - protein, 15 aa, Borrelia burgdorferi
TIYISWNFFAPIKAS

SEQ ID NO: 1086 - protein, 15 aa, Borrelia burgdorferi
IYISWNFFAPIKASF

SEQ ID NO: 1087 - protein, 15 aa, Borrelia burgdorferi
YISWNFFAPIKASFE

SEQ ID NO: 1088 - protein, 15 aa, Borrelia burgdorferi
ISWNFFAPIKASFEN

SEQ ID NO: 1089 - protein, 15 aa, Borrelia burgdorferi
SWNFFAPIKASFENN

SEQ ID NO: 1090 - protein, 15 aa, Borrelia burgdorferi
WNFFAPIKASFENNK

SEQ ID NO: 1091 - protein, 15 aa, Borrelia burgdorferi
NFFAPIKASFENNKD

SEQ ID NO: 1092 - protein, 15 aa, Borrelia burgdorferi
PIKASFENNKDTNYD

SEQ ID NO: 1093 - protein, 15 aa, Borrelia burgdorferi
IKASFENNKDTNYDL

SEQ ID NO: 1094 - protein, 15 aa, Borrelia burgdorferi
KASFENNKDTNYDLI

SEQ ID NO: 1095 - protein, 15 aa, Borrelia burgdorferi
NNKDTNYDLIINRKT

SEQ ID NO: 1096 - protein, 15 aa, Borrelia burgdorferi
NKDTNYDLIINRKTK

SEQ ID NO: 1097 - protein, 15 aa, Borrelia burgdorferi
KDTNYDLIINRKTKK

SEQ ID NO: 1098 - protein, 9 aa, Borrelia burgdorferi
IFAQTIDDE

SEQ ID NO: 1099 - protein, 9 aa, Borrelia burgdorferi
FAQTIDDEN

SEQ ID NO: 1100 - protein, 9 aa, Borrelia burgdorferi
IDDENSKKR

SEQ ID NO: 1101 - protein, 9 aa, Borrelia burgdorferi
LTLSQKSYL

SEQ ID NO: 1102 - protein, 9 aa, Borrelia burgdorferi
LSTDEDLKK

SEQ ID NO: 1103 - protein, 9 aa, Borrelia burgdorferi
LKKWALKEG

SEQ ID NO: 1104 - protein, 9 aa, Borrelia burgdorferi
WALKEGLKE

SEQ ID NO: 1105 - protein, 9 aa, Borrelia burgdorferi
LKETDVSKI

SEQ ID NO: 1106 - protein, 9 aa, Borrelia burgdorferi
VSKIRELLL

SEQ ID NO: 1107 - protein, 9 aa, Borrelia burgdorferi
IRELLLKKF

SEQ ID NO: 1108 - protein, 9 aa, Borrelia burgdorferi
FGIDPELFV

SEQ ID NO: 1109 - protein, 9 aa, Borrelia burgdorferi
LFVKGKGLA

SEQ ID NO: 1110 - protein, 9 aa, Borrelia burgdorferi
FVKGKGLAG

SEQ ID NO: 1111 - protein, 9 aa, Borrelia burgdorferi
LAGSGRYKI

SEQ ID NO: 1112 - protein, 9 aa, Borrelia burgdorferi
YKIIIETAD

SEQ ID NO: 1113 - protein, 9 aa, Borrelia burgdorferi
IIIETADNL

SEQ ID NO: 1114 - protein, 9 aa, Borrelia burgdorferi
IIETADNLE

SEQ ID NO: 1115 - protein, 9 aa, Borrelia burgdorferi
IETADNLEN

SEQ ID NO: 1116 - protein, 9 aa, Borrelia burgdorferi
YGLTKDESI

SEQ ID NO: 1117 - protein, 9 aa, Borrelia burgdorferi
LTKDESIIF

SEQ ID NO: 1118 - protein, 9 aa, Borrelia burgdorferi
IIFEGRVNI

SEQ ID NO: 1119 - protein, 9 aa, Borrelia burgdorferi
FEGRVNILV

SEQ ID NO: 1120 - protein, 9 aa, Borrelia burgdorferi
LVEDIKENK

SEQ ID NO: 1121 - protein, 9 aa, Borrelia burgdorferi
VNILVEDIK

SEQ ID NO: 1122 - protein, 9 aa, Borrelia burgdorferi
ILVEDIKEN

SEQ ID NO: 1123 - protein, 9 aa, Borrelia burgdorferi
IKENKKHNI

SEQ ID NO: 1124 - protein, 9 aa, Borrelia burgdorferi
IKGDRIVLN

SEQ ID NO: 1125 - protein, 9 aa, Borrelia burgdorferi
IVLNKNSKK

SEQ ID NO: 1126 - protein, 9 aa, Borrelia burgdorferi
VLNKNSKKL

SEQ ID NO: 1127 - protein, 9 aa, Borrelia burgdorferi
LNKNSKKLY

SEQ ID NO: 1128 - protein, 9 aa, Borrelia burgdorferi
LYAIGNVEY

SEQ ID NO: 1129 - protein, 9 aa, Borrelia burgdorferi
YAIGNVEYI

SEQ ID NO: 1130 - protein, 9 aa, Borrelia burgdorferi
YILDMDTNE

SEQ ID NO: 1131 - protein, 9 aa, Borrelia burgdorferi
VEYILDMDT

SEQ ID NO: 1132 - protein, 9 aa, Borrelia burgdorferi
LDMDTNEKL

SEQ ID NO: 1133 - protein, 9 aa, Borrelia burgdorferi
ILDMDTNEK

SEQ ID NO: 1134 - protein, 9 aa, Borrelia burgdorferi
MDTNEKLYF

SEQ ID NO: 1135 - protein, 9 aa, Borrelia burgdorferi
LYFYGNEFL

SEQ ID NO: 1136 - protein, 9 aa, Borrelia burgdorferi
YFYGNEFLV

SEQ ID NO: 1137 - protein, 9 aa, Borrelia burgdorferi
FLVDFDSQN

SEQ ID NO: 1138 - protein, 9 aa, Borrelia burgdorferi
LVDFDSQNF

SEQ ID NO: 1139 - protein, 9 aa, Borrelia burgdorferi
VDFDSQNFL

SEQ ID NO: 1140 - protein, 9 aa, Borrelia burgdorferi
FLLKNGILQ

SEQ ID NO: 1141 - protein, 9 aa, Borrelia burgdorferi
IDHILSFGG

SEQ ID NO: 1142 - protein, 9 aa, Borrelia burgdorferi
ILSFGGKVL

SEQ ID NO: 1143 - protein, 9 aa, Borrelia burgdorferi
VLKKIDNDV

SEQ ID NO: 1144 - protein, 9 aa, Borrelia burgdorferi
LKKIDNDVT

SEQ ID NO: 1145 - protein, 9 aa, Borrelia burgdorferi
IDNDVTILE

SEQ ID NO: 1146 - protein, 9 aa, Borrelia burgdorferi
VTILEQAFA

SEQ ID NO: 1147 - protein, 9 aa, Borrelia burgdorferi
FATTSKIPE

SEQ ID NO: 1148 - protein, 9 aa, Borrelia burgdorferi
QAFATTSKI

SEQ ID NO: 1149 - protein, 9 aa, Borrelia burgdorferi
PEPYYSIKA

SEQ ID NO: 1150 - protein, 9 aa, Borrelia burgdorferi
YYSIKASKI

SEQ ID NO: 1151 - protein, 9 aa, Borrelia burgdorferi
IKASKIWAL

SEQ ID NO: 1152 - protein, 9 aa, Borrelia burgdorferi
IWALPSGDF

SEQ ID NO: 1153 - protein, 9 aa, Borrelia burgdorferi
FGFLNAIFY

SEQ ID NO: 1154 - protein, 9 aa, Borrelia burgdorferi
IFYMGRVPV

SEQ ID NO: 1155 - protein, 9 aa, Borrelia burgdorferi
FFFRPGDSL

SEQ ID NO: 1156 - protein, 9 aa, Borrelia burgdorferi
FRPGDSLFF

SEQ ID NO: 1157 - protein, 9 aa, Borrelia burgdorferi
LFFNPSLGL

SEQ ID NO: 1158 - protein, 9 aa, Borrelia burgdorferi
LNPRKGFAV

SEQ ID NO: 1159 - protein, 9 aa, Borrelia burgdorferi
FAVFNTVYL

SEQ ID NO: 1160 - protein, 9 aa, Borrelia burgdorferi
VYLFGNKSS

SEQ ID NO: 1161 - protein, 9 aa, Borrelia burgdorferi
YLFGNKSSS

SEQ ID NO: 1162 - protein, 9 aa, Borrelia burgdorferi
FGNKSSSED

SEQ ID NO: 1163 - protein, 9 aa, Borrelia burgdorferi
LDFDFNSVY

SEQ ID NO: 1164 - protein, 9 aa, Borrelia burgdorferi
FDFNSVYNS

SEQ ID NO: 1165 - protein, 9 aa, Borrelia burgdorferi
FNSVYNSGK

SEQ ID NO: 1166 - protein, 9 aa, Borrelia burgdorferi
VYNSGKKPY

SEQ ID NO: 1167 - protein, 9 aa, Borrelia burgdorferi
YNSGKKPYI

SEQ ID NO: 1168 - protein, 9 aa, Borrelia burgdorferi
YIRNGYLTY

SEQ ID NO: 1169 - protein, 9 aa, Borrelia burgdorferi
LTYFFAENL

SEQ ID NO: 1170 - protein, 9 aa, Borrelia burgdorferi
YFFAENLAP

SEQ ID NO: 1171 - protein, 9 aa, Borrelia burgdorferi
FFAENLAPS

SEQ ID NO: 1172 - protein, 9 aa, Borrelia burgdorferi
FAENLAPSV

SEQ ID NO: 1173 - protein, 9 aa, Borrelia burgdorferi
VNKDYVKLI

SEQ ID NO: 1174 - protein, 9 aa, Borrelia burgdorferi
VKLIFDIYA

SEQ ID NO: 1175 - protein, 9 aa, Borrelia burgdorferi
IFDIYANLG

SEQ ID NO: 1176 - protein, 9 aa, Borrelia burgdorferi
FDIYANLGF

SEQ ID NO: 1177 - protein, 9 aa, Borrelia burgdorferi
LGFYSGIDF

SEQ ID NO: 1178 - protein, 9 aa, Borrelia burgdorferi
IDFNLGNTL

SEQ ID NO: 1179 - protein, 9 aa, Borrelia burgdorferi
LGHFKTLEG

SEQ ID NO: 1180 - protein, 9 aa, Borrelia burgdorferi
FKTLEGNFG

SEQ ID NO: 1181 - protein, 9 aa, Borrelia burgdorferi
LGFTRNIYS

SEQ ID NO: 1182 - protein, 9 aa, Borrelia burgdorferi
IYSYDGGYY

SEQ ID NO: 1183 - protein, 9 aa, Borrelia burgdorferi
YSYDGGYYP

SEQ ID NO: 1184 - protein, 9 aa, Borrelia burgdorferi
YYPFDNRTL

SEQ ID NO: 1185 - protein, 9 aa, Borrelia burgdorferi
LFSFSNLNK

SEQ ID NO: 1186 - protein, 9 aa, Borrelia burgdorferi
FRYLLKFKT

SEQ ID NO: 1187 - protein, 9 aa, Borrelia burgdorferi
LLKFKTEFL

SEQ ID NO: 1188 - protein, 9 aa, Borrelia burgdorferi
FLLSDALFL

SEQ ID NO: 1189 - protein, 9 aa, Borrelia burgdorferi
LFLVVLEHY

SEQ ID NO: 1190 - protein, 9 aa, Borrelia burgdorferi
FLVVLEHYS

SEQ ID NO: 1191 - protein, 9 aa, Borrelia burgdorferi
LVVLEHYSD

SEQ ID NO: 1192 - protein, 9 aa, Borrelia burgdorferi
LEHYSDPYV

SEQ ID NO: 1193 - protein, 9 aa, Borrelia burgdorferi
VNIDFRDRI

SEQ ID NO: 1194 - protein, 9 aa, Borrelia burgdorferi
IDFRDRIES

SEQ ID NO: 1195 - protein, 9 aa, Borrelia burgdorferi
FSLLNLDKD

SEQ ID NO: 1196 - protein, 9 aa, Borrelia burgdorferi
LNLDKDSVK

SEQ ID NO: 1197 - protein, 9 aa, Borrelia burgdorferi
VKEQTSIST

SEQ ID NO: 1198 - protein, 9 aa, Borrelia burgdorferi
LSSFYKRTF

SEQ ID NO: 1199 - protein, 9 aa, Borrelia burgdorferi
YKRTFNDGS

SEQ ID NO: 1200 - protein, 9 aa, Borrelia burgdorferi
ILDYKLNNL

SEQ ID NO: 1201 - protein, 9 aa, Borrelia burgdorferi
LDYKLNNLG

SEQ ID NO: 1202 - protein, 9 aa, Borrelia burgdorferi
LSGYENLYV

SEQ ID NO: 1203 - protein, 9 aa, Borrelia burgdorferi
LYVKSPLEK

SEQ ID NO: 1204 - protein, 9 aa, Borrelia burgdorferi
WFYLERIYA

SEQ ID NO: 1205 - protein, 9 aa, Borrelia burgdorferi
YAPYIDLNF

SEQ ID NO: 1206 - protein, 9 aa, Borrelia burgdorferi
LNFQKDLYN

SEQ ID NO: 1207 - protein, 9 aa, Borrelia burgdorferi
WTFSTDTKE

SEQ ID NO: 1208 - protein, 9 aa, Borrelia burgdorferi
FSTDTKEMI

SEQ ID NO: 1209 - protein, 9 aa, Borrelia burgdorferi
MRPEIKNLE

SEQ ID NO: 1210 - protein, 9 aa, Borrelia burgdorferi
IKNLEDKDN

SEQ ID NO: 1211 - protein, 9 aa, Borrelia burgdorferi
TKLTRDLYI

SEQ ID NO: 1212 - protein, 9 aa, Borrelia burgdorferi
ITLKNIDQS

SEQ ID NO: 1213 - protein, 9 aa, Borrelia burgdorferi
IDQSDSFFI

SEQ ID NO: 1214 - protein, 9 aa, Borrelia burgdorferi
FIRFGINPY

SEQ ID NO: 1215 - protein, 9 aa, Borrelia burgdorferi
IRFGINPYL

SEQ ID NO: 1216 - protein, 9 aa, Borrelia burgdorferi
INPYLRNNV

SEQ ID NO: 1217 - protein, 9 aa, Borrelia burgdorferi
YLRNNVFFD

SEQ ID NO: 1218 - protein, 9 aa, Borrelia burgdorferi
NVFFDNYGI

SEQ ID NO: 1219 - protein, 9 aa, Borrelia burgdorferi
FFDNYGITS

SEQ ID NO: 1220 - protein, 9 aa, Borrelia burgdorferi
YGITSPKDF

SEQ ID NO: 1221 - protein, 9 aa, Borrelia burgdorferi
FNYQIKNYL

SEQ ID NO: 1222 - protein, 9 aa, Borrelia burgdorferi
YQIKNYLFD

SEQ ID NO: 1223 - protein, 9 aa, Borrelia burgdorferi
YLFDIKNKT

SEQ ID NO: 1224 - protein, 9 aa, Borrelia burgdorferi
LFDIKNKTD

SEQ ID NO: 1225 - protein, 9 aa, Borrelia burgdorferi
IKNKTDIKI

SEQ ID NO: 1226 - protein, 9 aa, Borrelia burgdorferi
FDIKNKTDI

SEQ ID NO: 1227 - protein, 9 aa, Borrelia burgdorferi
NKTDIKIHA

SEQ ID NO: 1228 - protein, 9 aa, Borrelia burgdorferi
IKIHADFYN

SEQ ID NO: 1229 - protein, 9 aa, Borrelia burgdorferi
IHADFYNRL

SEQ ID NO: 1230 - protein, 9 aa, Borrelia burgdorferi
YNRLITFEN

SEQ ID NO: 1231 - protein, 9 aa, Borrelia burgdorferi
LITFENLLY

SEQ ID NO: 1232 - protein, 9 aa, Borrelia burgdorferi
LLYLNTIEY

SEQ ID NO: 1233 - protein, 9 aa, Borrelia burgdorferi
IEYSPLNKD

SEQ ID NO: 1234 - protein, 9 aa, Borrelia burgdorferi
YSPLNKDFK

SEQ ID NO: 1235 - protein, 9 aa, Borrelia burgdorferi
LNKDFKVED

SEQ ID NO: 1236 - protein, 9 aa, Borrelia burgdorferi
FKVEDKDKK

SEQ ID NO: 1237 - protein, 9 aa, Borrelia burgdorferi
IINQINLNL

SEQ ID NO: 1238 - protein, 9 aa, Borrelia burgdorferi
INQINLNLL

SEQ ID NO: 1239 - protein, 9 aa, Borrelia burgdorferi
LNLLPFIRY

SEQ ID NO: 1240 - protein, 9 aa, Borrelia burgdorferi
LLPFIRYPL

SEQ ID NO: 1241 - protein, 9 aa, Borrelia burgdorferi
LFSRSTLKF

SEQ ID NO: 1242 - protein, 9 aa, Borrelia burgdorferi
LKFENKATL

SEQ ID NO: 1243 - protein, 9 aa, Borrelia burgdorferi
FENKATLYS

SEQ ID NO: 1244 - protein, 9 aa, Borrelia burgdorferi
LYSFNKKYD

SEQ ID NO: 1245 - protein, 9 aa, Borrelia burgdorferi
YSFNKKYDS

SEQ ID NO: 1246 - protein, 9 aa, Borrelia burgdorferi
YDSDVKSLV

SEQ ID NO: 1247 - protein, 9 aa, Borrelia burgdorferi
VKSLVNKNS

SEQ ID NO: 1248 - protein, 9 aa, Borrelia burgdorferi
VNKNSSIFL

SEQ ID NO: 1249 - protein, 9 aa, Borrelia burgdorferi
FLSDPETFY

SEQ ID NO: 1250 - protein, 9 aa, Borrelia burgdorferi
FYQSLTASL

SEQ ID NO: 1251 - protein, 9 aa, Borrelia burgdorferi
YQSLTASLI

SEQ ID NO: 1252 - protein, 9 aa, Borrelia burgdorferi
YDYFTTELS

SEQ ID NO: 1253 - protein, 9 aa, Borrelia burgdorferi
FTTELSGEL

SEQ ID NO: 1254 - protein, 9 aa, Borrelia burgdorferi
LSGELKNSF

SEQ ID NO: 1255 - protein, 9 aa, Borrelia burgdorferi
FEDIKASSE

SEQ ID NO: 1256 - protein, 9 aa, Borrelia burgdorferi
IKASSELKL

SEQ ID NO: 1257 - protein, 9 aa, Borrelia burgdorferi
LSLDFPYLL

SEQ ID NO: 1258 - protein, 9 aa, Borrelia burgdorferi
YLLQEAGIG

SEQ ID NO: 1259 - protein, 9 aa, Borrelia burgdorferi
LLQEAGIGI

SEQ ID NO: 1260 - protein, 9 aa, Borrelia burgdorferi
IKYYKKFKE

SEQ ID NO: 1261 - protein, 9 aa, Borrelia burgdorferi
YYKKFKEDA

SEQ ID NO: 1262 - protein, 9 aa, Borrelia burgdorferi
YKKFKEDAM

SEQ ID NO: 1263 - protein, 9 aa, Borrelia burgdorferi
ISAVQSPLE

SEQ ID NO: 1264 - protein, 9 aa, Borrelia burgdorferi
YKNLEMSPA

SEQ ID NO: 1265 - protein, 9 aa, Borrelia burgdorferi
LEMSPALYY

SEQ ID NO: 1266 - protein, 9 aa, Borrelia burgdorferi
YYKIEPRYL

SEQ ID NO: 1267 - protein, 9 aa, Borrelia burgdorferi
YKIEPRYLD

SEQ ID NO: 1268 - protein, 9 aa, Borrelia burgdorferi
LYYKIEPRY

SEQ ID NO: 1269 - protein, 9 aa, Borrelia burgdorferi
FLVAYDPLI

SEQ ID NO: 1270 - protein, 9 aa, Borrelia burgdorferi
VAYDPLINR

SEQ ID NO: 1271 - protein, 9 aa, Borrelia burgdorferi
YDPLINRVS

SEQ ID NO: 1272 - protein, 9 aa, Borrelia burgdorferi
LINRVSELS

SEQ ID NO: 1273 - protein, 9 aa, Borrelia burgdorferi
INRVSELSF

SEQ ID NO: 1274 - protein, 9 aa, Borrelia burgdorferi
FQFLFAMKD

SEQ ID NO: 1275 - protein, 9 aa, Borrelia burgdorferi
LFAMKDEFE

SEQ ID NO: 1276 - protein, 9 aa, Borrelia burgdorferi
FLFAMKDEF

SEQ ID NO: 1277 - protein, 9 aa, Borrelia burgdorferi
FEYNYDPLK

SEQ ID NO: 1278 - protein, 9 aa, Borrelia burgdorferi
YNYDPLKGD

SEQ ID NO: 1279 - protein, 9 aa, Borrelia burgdorferi
YDPLKGDFS

SEQ ID NO: 1280 - protein, 9 aa, Borrelia burgdorferi
LVPYSLDSS

SEQ ID NO: 1281 - protein, 9 aa, Borrelia burgdorferi
YSLDSSYKK

SEQ ID NO: 1282 - protein, 9 aa, Borrelia burgdorferi
VLTFFDNKL

SEQ ID NO: 1283 - protein, 9 aa, Borrelia burgdorferi
LTFFDNKLS

SEQ ID NO: 1284 - protein, 9 aa, Borrelia burgdorferi
LSFTLGVDV

SEQ ID NO: 1285 - protein, 9 aa, Borrelia burgdorferi
LQKFTDNEL

SEQ ID NO: 1286 - protein, 9 aa, Borrelia burgdorferi
LRSALTLKL

SEQ ID NO: 1287 - protein, 9 aa, Borrelia burgdorferi
LEIYFSTLS

SEQ ID NO: 1288 - protein, 9 aa, Borrelia burgdorferi
YFSTLSINT

SEQ ID NO: 1289 - protein, 9 aa, Borrelia burgdorferi
FKYFKGYMD

SEQ ID NO: 1290 - protein, 9 aa, Borrelia burgdorferi
FKGYMDQIG

SEQ ID NO: 1291 - protein, 9 aa, Borrelia burgdorferi
LEPVNVFAD

SEQ ID NO: 1292 - protein, 9 aa, Borrelia burgdorferi
VNVFADLLK

SEQ ID NO: 1293 - protein, 9 aa, Borrelia burgdorferi
FNFFNSQDR

SEQ ID NO: 1294 - protein, 9 aa, Borrelia burgdorferi
FFNSQDRKD

SEQ ID NO: 1295 - protein, 9 aa, Borrelia burgdorferi
FNSQDRKDS

SEQ ID NO: 1296 - protein, 9 aa, Borrelia burgdorferi
LFKIKKFSS

SEQ ID NO: 1297 - protein, 9 aa, Borrelia burgdorferi
WKFVGEYNL

SEQ ID NO: 1298 - protein, 9 aa, Borrelia burgdorferi
FVGEYNLEP

SEQ ID NO: 1299 - protein, 9 aa, Borrelia burgdorferi
YNLEPDLLI

SEQ ID NO: 1300 - protein, 9 aa, Borrelia burgdorferi
YSPIWRNNF

SEQ ID NO: 1301 - protein, 9 aa, Borrelia burgdorferi
WRNNFTIYI

SEQ ID NO: 1302 - protein, 9 aa, Borrelia burgdorferi
FTIYISWNF

SEQ ID NO: 1303 - protein, 9 aa, Borrelia burgdorferi
IYISWNFFA

SEQ ID NO: 1304 - protein, 9 aa, Borrelia burgdorferi
WNFFAPIKA

SEQ ID NO: 1305 - protein, 9 aa, Borrelia burgdorferi
FAPIKASFE

SEQ ID NO: 1306 - protein, 9 aa, Borrelia burgdorferi
FENNKDTNY

SEQ ID NO: 1307 - protein, 9 aa, Borrelia burgdorferi
TNYDLIINR

SEQ ID NO: 1308 - protein, 9 aa, Borrelia burgdorferi
YDLIINRKT

SEQ ID NO: 1309 - protein, 742 aa, Borrelia hermsii
MLKIKHRFVGFLFLCLVITILFFSLIFKFVLSNYLEHYYKQLTRAQARRAAFAMQALLDTFYIIIDGASSNLALETMDEYSSIKNGGQMFSEVELDMLRENSQIVLDSVKGYKKYRGRLYDILWDLKLDTVYEEFAFLDFEGKVIVTTRHANNMDFGQSESNTEYFKKSVEEYKKNKLSFIGWYSGLTEGIAGEVAVRSYYKDKKASAIVVPVYSAEDKVFLGYLVGYLLNDVIRDRLDRSRFGFYNRGNLFYLDPYNNLVNPLIEYNESSKLSAKFVDVLKDTLSKPPRQPIVSTEVPVYQIEKAYLPEMGLYNYYAILPISSELGENSGILVARIPYEDIHGVIDSLALKFIVSSVLGIIILVVILSTKVEKIISSRLNLIRNLVKEIVQGNLDKDCVIGNDKYFDELSSLGLQVIKMRNAIADAIASVLRNINYVNKASVEVANSSQNLSSSALQQASTLEEMSANIEQISAGVKMSAHNSQETERIALITNESAQIGGKAVEESVVAMQAIVEKVSVIEEIARKTNLLALNAAIEAARAGEEGKGFAVVASEIRKLADLSKESALEIGELVEENSRLATEAGLIFKDMLPEIEKTTNLVKRISEGSYKQNEQIMHFKMALDQVGEVVQASASSSEELSSMADRMLEKAKELKQVISFFKVKDSEVNISDAFNDEDDDFVTGSLATLNVESNPSLKDRQDDIHGDSNVSYESINKRVDPKKAIDVVDKNLDFDEDFSDF

SEQ ID NO: 1310 - protein, 753 aa, Borrelia afzelii
MLLKLKYRFVGFLLLFLIFILLLFSMIFNFVLCGYLEDYYKQLTRAQLRRAAFSTQSFLDTLYVIVSGAASNLALETISEFAIYENRGKDFSESELIDLRKNPKFVIDSVKVNKKYRQYLYNFMANLKNDTFFEEFAFFDFEGRIIVSTRHENNMDFGHSEANTNYFKKAVEDYKQDQLKFIGWYSNLSEGISAEVAIRSRQSEKKAFAIIVPVYSPEDKLVCGYLAGYLLNDVLADSFDRFRFGFYKRGNFIYVDPNNIAVNPFEEYNEISRVSSKFLNTLKDVFSKPPLLSNIPTEVSVYTIDRIFFPEMGEECYYALLPISSKLGEKGGVLIARLPYKDIYGVISSIRFQYVLYSALGIILLSIVLLARIDRVISFRLNAIRVLVQDMVNGSLDKDYALDDGDDFSDELSMLSLRVVKMKKAISVAIASVLRNISYVNKASLEVASSSQNLSSSALQQASALEEMSANVEQIASGVNMSANNSYETEQIALKTNENSHIGGKAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENLEDGEYDFRLMDCPEDSFKDENQDLKSNGISTLNGSRHNNHSLSIESEPSVRAINKRVDPKKAIDIADKDLNFDDDFSEF

SEQ ID NO: 1311 - protein, 753 aa, Borrelia burgdorferi
MLLKLKYRFVGFLLLFLIFILLLFSTIFNFVLCGYLEDYYKQLTRAQVRRAAFSLQSFLDTLHVIINGAASNLALETISEFAMSENRGKDFSESELIDLRKNPKFVIDSVKVSKKYRQYLYNFMANLKNDTLFEEFAFFDFEGRVIVSTRHENNMDFGHSEANTNYFKKAVEDYRQNQLKFIGWYSNLSEGISAEVAIRSKQSEKKAFAIIVPVYSPEDKLVCGYLAGYLLNDIVADSFDRFRFGFYKRGNFIYVDPNNIAVNPFEEYNETSRVSSKFLNVLKDVFSKPPFPSNIASEVSVYTIDRILLSEMGEDCYYAMLPISSKLGEKSGVLIARLPYKDIYGVISSLRFQYILYSVLGIIALSIVLSIRIDRVISFRLNAIRVLVQDMVKGNLDKDYALDDDENTLDELGMLSLQVVKMKKAISVAISSVLRNISYVNKASLEVASSSQNLSSSALQQASALEEMSANVEQIASGVNMSANNSYETEQIALKTNENSQIGGRAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENPENDDYDFRLIDCPENSFKDENQNLKSNGISTSNASGHNNYSLDIESESSVRTINKRVDPKKAIDIADKDLNFDDDFSEF

SEQ ID NO: 1312 - protein, 753 aa, Borrelia burgdorferi
MLLKLKYRFVGFLLLFLIFILLLFSTIFNFVLCGYLEDYYKQLTRAQVRRAAFSLQSFLDTLHVIINGAASNLALETISEFAMSENRGKDFSESELIDLRKNPKFVIDSVKVSKKYRQYLYNFMANLKNDTLFEEFAFFDFEGRIIVSTRHENNMDFGHSEANTNYFKKAVEDYRQNQLKFIGWYSNLSEGISAEVAIRSKQSEKKAFAIIVPVYSPEDKLVCGYLAGYLLNDIVADSFDRFRFGFYKRGNFIYVDPNNIAVNPFEEYNETSRVSSKFLNVLKDVFSKPPFPSNIASEVSVYTIDRILLSEMGEDCYYAMLPISSKLGEKSGVLIARLPYKDIYGVISSLRFQYILYSVLGIIALSIVLSIRIDRIISFRLNAIRVLVQDMVKGNLDKDYALDDDENTLDELGMLSLQVVKMKKAISVAISSVLRNISYVNKASLEVASSSQNLSSSALQQASALEEMSANVEQIASGVNMSANNSYETEQIALKTNENSQIGGRAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKVKDSKIENPENDDYDFRLVDCPENSFKDENQNLKSNGISTSNASGHNNYSLDIESESSVRTINKRVDPKKAIDIADKDLNFDDDFSEF

SEQ ID NO: 1313 - protein, 753 aa, Borrelia burgdorferi
MLLKLKYRFVGFLLLFLIFILLLFSTIFNFVLCGYLEDYYKQLTRAQVRRAAFSLQSFLDTLHVIINGAASNLALETISEFAMSENRGKDFSESELIDLRKNPKFVIDSVKVSKKYRQYLYNFMANLKNDTLFEEFAFFDFEGRVIVSTRHENNMDFGHSEANTNYFKKAVEDYRQNQLKFIGWYSNLSEGISAEVAIRSKQSEKKAFAIIVPVYSPEDKLVCGYLAGYLLNDIVADSFDRFRFGFYKRGNFIYVDPNNIAVNPFEEYNETSRVSSKFLNVLKDVFSKPPFPSNIASEVSVYTIDRILLSEMGEDCYYAMLPISSKLGEKSGVLIARLPYKDIYGVISSLRFQYILYSVLGIIALSIVLSIRIDRIISFRLNAIRVLVQDMVKGNLDKDYALDDDENTLDELGMLSLQVVKMKKAISVAISSVLRNISYVNKASLEVASSSQNLSSSALQQASALEEMSANVEQIASGVNMSANNSYETEQIALKTNENSQIGGRAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENPENDDYDFRLIDCPENSFKDENQNLKSNGISTSNASGHNNYSLDIESESSVRTINKRVDPKKAIDIADKDLNFDDDFSEF

SEQ ID NO: 1314 - protein, 753 aa, Borrelia burgdorferi
MLLKLKYRFVGFLLLFLIFILLLFSTIFNFVLCGYLEDYYKQLTRAQVRRAAFSLQSFLDTLHVIINGAASNLALETISEFAMSENRGKDFSESELIDLRKNPKFVIDSVKVSKKYRQYLYNFMANLKNDTLFEEFAFFDFEGRIIVSTRHENNMDFGHSEANTNYFKKAVEDYRQNQLKFIGWYSNLSEGISAEVAIRSKQSEKKAFAIIVPVYSPEDKLVCGYLAGYLLNDIVADSFDRFRFGFYKRGNFIYVDPNNIAVNPFEEYNETSRVSSKFLNVLKDVFSKPPFPSNIASEVSVYTIDRILLSEMGEDCYYAMLPISSKLGEKSGVLIARLPYKDIYGVISSLRFQYILYSVLGIIALSIVLSIRIDRIISFRLNAIRVLVQDMVKGNLDKDYALDDDENTLDELGMLSLQVVKMKKAISVAISSVLRNISYVNKASLEVASSSQNLSSSALQQASALEEMSANVEQIASGVNMSANNSYETEQIALKTNENSQIGGRAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENPENDDYDFRLIDCPENSFKDENQNLKSNGISTSNASGHNNYSLDIESESSVRTINKRVDPKKAIDIADKDLNFDDDFSEF

SEQ ID NO: 1315 - protein, 753 aa, Borrelia garinii
MLVKLKYRFVGFLLLFLIFILLLFSMIFNFVLCGYLEDYYKQLTRAQLRRAAFSTQSFLDTLYVIINGAASNLALETISEFAISENRGKDFSESELIDLRKNSKFVIDSVKVNKKYRQYLYNFMTNLKNDTLFEEFAFFDFEGRIIVSTRHENNMDFGHSEANTNYFKKAIEDYKQNQLKFIGWYSNLSEGISAEVAIRSRQSEKKAFAIIVPVYSPEDKLVCGYLAGYLLNDVLADSFDRFRFGFYKRGNFVYVDSNNIAVNPFEEYNETSRVSSKFLNILKDVFSKPPLLSNIPTEVSVYTIDRILFSEMGEECYYALLPISSKLGEKGGVLIARLPYKDIYGVISSLKFQYILYSVLGIISLSIVLLMRIDRIISFRLNAIRVLVQDMVKGDLDKDYALDDGDDCFDELGMLSLQVVKMKKAISVAIASVLRNISYVNKASLEVASSSQNLSSSALQQASALEEMSANVEQIASGVNMSANNSYETEQIALKTNENSQIGGKAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENLEDGDYDFRLMDCPENSFKDENQDLKGNEISTLNGSRHSNYSLSIESESSARTINKRVDPKKAIDIADKDLNFDDDFSEF

SEQ ID NO: 1316 - protein, 753 aa, Borrelia garinii
MLVKLKYRFVGFLLLFLIFILLLFSMIFNFVLCGYLEDYYKQLTRAQLRRAAFSTQSFLDTLYVIINGAASNLALETISEFAISENRGKDFSESELVDLRKNSKFVIDSVKVNKKYRQYLYNFMTNLKNDTLFEEFAFFDFEGRIIVSTRHENNMDFGHSEANTNYFKKAIEDYKQNQLKFIGWYSNLSEGISAEVAIRSRQSEKKAFAIIVPVYSPEDKLVCGYLAGYLLNDVLADSFDRFRFGFYKRGNFVYIDSNNIAVNPFEEYNETSRVSSKFLNILKDVFSKPPLLSNIPTEVSVYTIDRILFSEMGEECYYALLPISSKLGEKGGVLIARLPYKDIYGVISSLKFQYILYSVLGIISLSIVLLMRIDRIISFRLNAIRVLVQDMVKGDLDKDYALDDGDDYFDELGMLSLQVVKMKKAISVAIASVLRNISYVNKASLEVASSSQNLTSSALQQASALEEMSANVEQIASGVNMSANNSYETEQIALKTNENSQIGGKAVEESVIAMQDIVEKVSVIEEIARKTNLLALNAAIEAARAGDEGKGFAVVASEIRKLADLSKISALEIGELVEDNSKVATEAGVIFKEMLPEIEETANLVKKISEGSSKQSDQIAQFKMALDQVGEVVQSSASSSEQLSSMSDKMLEKSKELRKSVLFFKIKDSKIENLEDGDYDFRLMDCPENSFKDENQDLKGNEISTLNGSRHSNYSLSIESEPSARTINKRVDPKKAIDIADKDLNFDDDFSEF

SEQ ID NO: 1317 - protein, 752 aa, unidentified
MLLKLKYRFVGFLLLFLIFILLLFSMIFNFVLCGYLEDYYKQLTRAQVRRAAFSTQSFLDTLYVIINGAASNLALETISEFAISENRGKDFSESELVDLRKNSKFVIDSVKVSKKYRQYLYNFMSNLKNDTFFEEFAFFDFEGRIIVSTRHENNMDFGHSEANTNYFKKAVEDYKQHQLKFIGWYSNLSEGISAEVAIRSKQSEKKAFAIIVPVYSPEDKLVCGYLAGYLLNDVLADSFDRFRFGFYKRGNFIYVDPNNIAVNPFEEYNETSRVSSKFLNVLKDVFSKPPLSSNVSTEVSVYTIDRIFLSEMEEECYYALLPISSKLGDKSGVLIARLPYKDIYGVISSLRFQYILYSVLGIVVLSLVLLIRIDKIISFRLNAIRVLVQDMVKGNLDKDYALDDDNVFDELGMLSLQVVKMKKAISVAIASVLRNISYVNKASLEVASSSQNLSSSALQQASALEEMSANVEQIASGVNMSANNSYETEQIALKTNENSHIGGKAVEESVIAMQDIVEKVSVIEEIARKTNLLALNAAIEAARAGDEGKGFAVVASEIRKLADLSKISALEIGELVEDNSRVATEAGVIFKEMLPEIEETANLVKKISEGSSKQSDQIAQFKMALDQVGEVVQSSASSSEQLSSMSDKMLEKSKELRKSVLFFKIKDSTIENLEEDDYDFRLMDCLENSFKDENKNLKSNGISTLNGSRHNNHSLDIESEPSVRTINKRVDPKKAIDIADKDLNFDDDFSEF

SEQ ID NO: 1318 - protein, 50 aa, Borrelia burgdorferi
NVFDFQFLFAMKDDFEYNYDPLKGDFSKIGTTTKLVPYSLDSSYKKELYV

SEQ ID NO: 1319 - protein, 50 aa, unidentified
NVFDFQFLFAMKDDFEYNYDPLKGDFFKVGTTTKLVPYSLDSSYKKELYV

SEQ ID NO: 1320 - protein, 50 aa, unidentified
NVFDFQFLFAMKDDFEYNYDSLKGDFSKIGTTTKLVPYSFDSSYKKELYV

SEQ ID NO: 1321 - protein, 50 aa, Borrelia afzelii
NVFDFQVLFAMKDDFEYNYDPLKGDFSKVGIATKLVPYSLDSSYKKEFNV

SEQ ID NO: 1322 - protein, 50 aa, Borrelia garinii
NVFDFQFLFAMKDDFEYNYDPLKGDFSKVGITTKLVPYSLDSRYKKEFHV

SEQ ID NO: 1323 - protein, 50 aa, unidentified
NGFDFQVLFAMKDDFEYNYDPLKGDFSKVGITTKLVPYSLDSSYKKEFHV

SEQ ID NO: 1324 - protein, 12 aa, synthetic construct
  SITE 6: note = Deamidated residue
  SITE 11: note = Oxidated residue
NVFDFQFLFAMK

SEQ ID NO: 1325 - protein, 12 aa, synthetic construct
  SITE 11: note = Oxidated residue
NVFDFQFLFAMK

(19) United States
(12) Patent Application Publication
(10) Pub. No.: US 2026/0365702 A1
(43) Pub. Date: Nov. 19, 2026

(54) Use of Immunogenic T Cell Epitopes for Lyme Disease Vaccination and Diagnosis

(71) Applicant: The Medical College of Wisconsin, Inc., Milwaukee, WI (US)

(72) Inventors: Robert B. Lochhead, Germantown, WI (US); Rebecca L. Danner, Milwaukee, WI (US); Joseph R. Rouse, Milwaukee, WI (US); Lauren Prochniak, Kenosha, WI (US)

(21) Appl. No.: 19/108,084

(22) PCT Filed: Sep. 1, 2023
(86) PCT No.: PCT/US2023/073311

§ 371 Date: Feb. 28, 2025

Related U.S. Application Data
(60) Provisional application No. 63/403,451, filed on Sep. 2, 2022.
(60) Provisional application No. 63/501,445, filed on May 11, 2023.

(51) Int. Cl. C07K 14/20; A61K 39/02; A61P 37/04; G01N 33/569

(52) U.S. Cl. CPC C07K 14/20; A61K 39/02; A61P 37/04; G01N 33/56911; G01N 2333/20

(57) ABSTRACT

Disclosed herein are compositions and methods for treating, and for the preventative treatment of Lyme disease. Also disclosed herein are compositions and methods for detecting and diagnosing infection by a Borrelia sp.

### CROSS-REFERENCE TO RELATED APPLICATIONS

This application claims priority to U.S. application No. 63/403,451 filed on Sep. 2, 2022, and to U.S. application No. 63/501,445 filed on May 11, 2023. The entire contents of each of the aforementioned applications is incorporated by reference herein.

### STATEMENT REGARDING FEDERALLY SPONSORED RESEARCH OR DEVELOPMENT

This invention was made with government support under R21AI148982 awarded by the National Institute of Allergy and Infectious Disease. The government has certain rights in the invention.

### SEQUENCE LISTING

A Sequence Listing accompanies this application and is submitted as an XML file of the sequence listing named “650053_01002_SL” which is 1,137,000 bytes in size and was created on Aug. 28, 2023. The sequence listing is electronically submitted via Patent Center with the application and is incorporated herein by reference in its entirety.

### BACKGROUND

Lyme disease is the most common vector-borne disease in the United States. Lyme disease is caused by the bacterium Borrelia burgdorferi, Borrelia garinii, Borrelia afzeli, Borrelia bavariensis, Borrelia mayonii and others. It is transmitted to humans through the bite of infected blacklegged ixodes ticks. Typical symptoms include fever, headache, fatigue, and a characteristic skin rash called erythema migrans. If left untreated, infection can spread to joints, the heart, and the nervous system. Lyme disease is diagnosed based on symptoms, physical findings (e.g., rash), and the possibility of exposure to infected ticks.

### SUMMARY

Disclosed herein are compositions and methods for the treatment and/or preventative treatment of Lyme disease in a subject. Also disclosed are methods for detecting Lyme disease in a subject in need thereof, or detecting exposure to Borrelia spp.

In certain embodiments, disclosed herein are polypeptides comprising or consisting of fragments of BB0680 (MCP4) or variants thereof. In some embodiments, the polypeptides comprise or consist of one or more of the polypeptides presented in Tables 1 and 2. In some embodiments, the polypeptide comprises or consists of SEQ ID NO: 1, or a polypeptide at least about 90% identical to SEQ ID NO: 1.

Also disclosed herein are compositions comprising the polypeptides of the present disclosure. In some embodiments, the composition comprises a polypeptide comprising or consisting of SEQ ID NO: 1, and optionally an adjuvant. In some embodiments, the composition comprises one or more additional polypeptides, such as OspA, OspC, or one or more polypeptides listed in Tables 1 and 2.

In various embodiments, disclosed herein are polypeptides comprising or consisting of fragments of BB0838 (LptD) or variants thereof. In some embodiments, the polypeptides comprise or consist of one or more of the polypeptides presented in Tables 3 and 4. In some embodiments, the polypeptide comprises or consists of SEQ ID NO: 2, or a polypeptide at least about 90% identical to SEQ ID NO: 2.

Also disclosed herein are compositions comprising the polypeptides of the present disclosure. In some embodiments, the composition comprises a polypeptide comprising or consisting of SEQ ID NO: 2, and optionally an adjuvant. In some embodiments, the composition comprises one or more additional polypeptides, such as OspA, OspC, or one or more polypeptides in listed in Tables 3 and 4.

In certain embodiments, the compositions disclosed herein can include one or more polypeptides disclosed herein, such as a polypeptide comprising or consisting of SEQ ID NO:1, a polypeptide at least about 90% identical to SEQ ID NO: 1, a polypeptide comprising or consisting of SEQ ID NO: 2, a polypeptide at least about 90% identical to SEQ ID NO: 2, one or more polypeptides listed in Tables 1, 2, 3, 4, and 5, or a combination thereof.

In various embodiments, the composition disclosed herein can include one or more of: a polypeptide at least 90% or 95% identical to SEQ ID NO: 1, a polypeptide at least 90% or 95% identical to SEQ ID NO: 2, a polypeptide at least 90% or 95% identical to SEQ ID NO: 3, or a polypeptide at least 90% or 95% identical to SEQ ID NO: 4.

Also disclosed herein are methods of treating Lyme disease, or a method for the preventative treatment of Lyme disease, the method comprising: administering to a subject the polypeptide or composition as disclosed herein. In some embodiments, the subject is asymptomatic for Lyme disease, but may have come in contact with a blacklegged tick selected from Ixodes scapularis or Ixodes pacificus. In some embodiments, the subject is asymptomatic. In some embodiments the subject is symptomatic, and exhibits one or more of the following symptoms: erythema migrans, facial palsy, and arthritis; and/or the subject tests positive for Lyme disease in standard two-tier test (STT).

Also disclosed herein are methods for testing a subject for exposure to Lyme disease, the method comprising: contacting a subject sample to one or more polypeptides disclosed herein. In some embodiments, the one or more polypeptides comprise or consist of SEQ ID NO: 1, a polypeptide at least about 90% identical to SEQ ID NO: 1, a polypeptide comprising or consisting of SEQ ID NO: 2, a polypeptide at least about 90% identical to SEQ ID NO: 2, or a combination thereof. In some embodiments, the one or more polypeptides is linked to a solid support.

Also disclosed herein are kits comprising one or more polypeptides as disclosed herein, and optionally instructions for use of the kit. In some embodiments, the one or more polypeptides is linked to a solid support, and comprise or consist of SEQ ID NO:1, a polypeptide at least about 90% identical to SEQ ID NO: 1, a polypeptide comprising or consisting of SEQ ID NO: 2, a polypeptide at least about 90% identical to SEQ ID NO: 2, or a combination thereof.

### BRIEF DESCRIPTION OF THE FIGURES

FIG. 1. Overview of methodology used to identify immunogenic MHC II peptides in mice infected with Borrelia burgdorferi.

FIG. 2A-2B. Predicted structure of BB0680 (MCP4), with MCP₄₄₂₋₄₆₂ epitope (Peptide 1) highlighted within the methyl-accepting transducer domain.

FIG. 3. Percent identify of MCP4 protein in various Borrelia strains, including Bb sensu stricto, Bb sensu lato, and non-Lyme disease spirochete Borrelia hermsii, a relapsing fever spirochete.

FIG. 4A-4B. CD4+ T cell epitope MCP₄₄₂₋₄₆₂ (Peptide 1) is immunogenic in mice infected with B. burgdorferi. Lymphocytes from mice infected with B. Burgdorferi for 4 weeks were stained with a proliferation dye (Cell-Trace Violet) and stimulated with Peptide 1 or OVA peptide as a negative control and incubated for 5 days. (A) Flow cytometric analysis of CD4+ T cells identified a population of activated, proliferating CD4+ T cells in lymphocytes stimulated with Peptide 1, but not with OVA peptide. (B) Analysis of CD4− lymphocytes, which include CD8+ T cells and B cells, also identified a population of activated lymphocytes. Representative of 2 biological replicates.

FIG. 5. CLUSTAL O(1.2.4) multiple sequence alignment of BB0680 (MCP4). B. hermsii is a relapsing fever Borrelia. B. afzelii, B. garinii, B. garinii-bonariensis, and B. mayonii are Bb sensu lato strains. JD1, N40, B31, and ZS7 are isolates representing Bb sensu stricto strains. Peptide 1 is highlighted in bold. FIG. 5 includes SEQ ID NOS 1309-1317, respectively, in order of appearance from top to bottom.

FIG. 6. Table showing MHC class II peptides derived from Borrelia burgdorferi identified by LC/MS/MS. FIG. 6 discloses SEQ ID NOS 1324-1325, and 3-4, respectively, in order of appearance.

FIG. 7. Structure of LptD (BB0838) based on Phyre2 modeling. LptD₉₂₃₋₉₃₄ is at the interface between the central β-barrel and C-terminal β-barrel/plug domains. According to this model, LptD₉₂₃₋₉₃₄ (magenta) is localized to the cell surface, within the outer membrane (OM)-spanning β-barrel. The LptD N-terminal periplasmic domain consists of a β-jellyroll domain with an unresolved N-terminal sequence (cyan). The central domain of LptD consists of a β-barrel that is predicted to span the OM (green). The C-terminal domain, which includes LptD₉₂₃₋₉₃₄, consists of a β-barrel and an unresolved C-terminal sequence, possibly a plug (yellow).

FIG. 8. Sequence alignment and phylogenic analysis of LptD across Lyme disease spirochetes. ClustalW as used to align LptD protein sequences from Borrelia burgdorferi, Borrelia califorensis, Borrelia mayonii, Borrelia garinii, Borrelia afzelii, Borrelia baveriensis. The alignment and amino acid conservation of the 50 amino acids downstream from the LptD₉₂₃₋₉₃₄ epitope (underlined) is shown. Brackets indicate promiscuous core epitopes that are predicted to bind to a large number of HLA-DRB1 alleles (see FIGS. 11 and 12 for complete list). LptD sequence from relapsing fever spirochete Borrelia hermsii was used as an outgroup in generation of a phylogenetic relationship tree (below the alignment), with numbers indicating the Baysian distance from the consensus sequence. FIG. 8 discloses SEQ ID NOS 1318-1323, respectively, in order of appearance from top to bottom.

FIG. 9. Is a schematic depiction of a proposed use of T cell vaccine components for use in a novel Lyme disease vaccine.

### DETAILED DESCRIPTION

Lyme disease affects nearly 500,000 individuals in the U.S. annually and is caused by infection with the tick-borne spirochete Borrelia burgdorferi. Although most patients are effectively treated with antibiotic therapy, ~10% of patients with Lyme disease have persistent symptoms that continue for months to years following antibiotic therapy and apparent spirochetal killing, called post-treatment Lyme disease syndrome (PTLDS), sometimes referred to as “chronic Lyme disease.” Unfortunately, there are critical gaps in diagnostic and therapeutic tools available to clinicians to treat patients suffering from Lyme disease, particularly following antibiotic therapy. Therefore, novel diagnostic tools and effective vaccines are urgently needed.

T helper cells, also called CD4+ T cells, are the “generals” of the adaptive immune system. They regulate all aspects of immune responses to pathogens by directing other components of the immune system to find, neutralize, and kill pathogens during infection, and promote immune resolution and tissue repair post-infection. Dysregulated CD4+ T cell responses can also drive numerous chronic inflammatory, arthritic, neurodegenerative, and fibrotic diseases. B. burgdorferi has evolved mechanisms to evade CD4+ T cell responses during infection. This likely contributes to chronic infection, immune dysregulation, and ongoing tissue damage and fibrosis in Lyme disease patients, particularly the tens of thousands of individuals suffering from chronic Lyme disease/post-treatment Lyme disease syndrome (PTLDS), as well as coinfections. However, very little is known about this part of the adaptive immune system due, in large part, to a shortage of validated T cell immune targets.

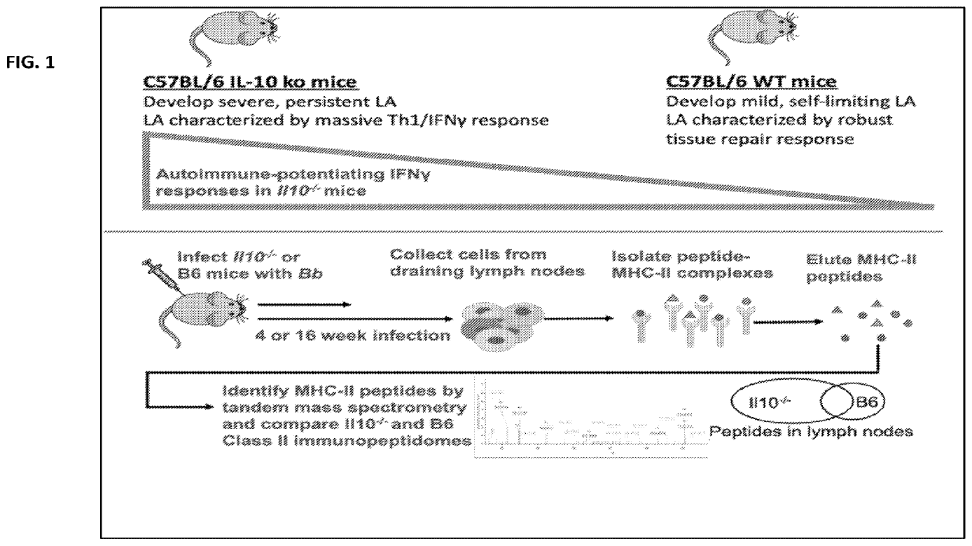

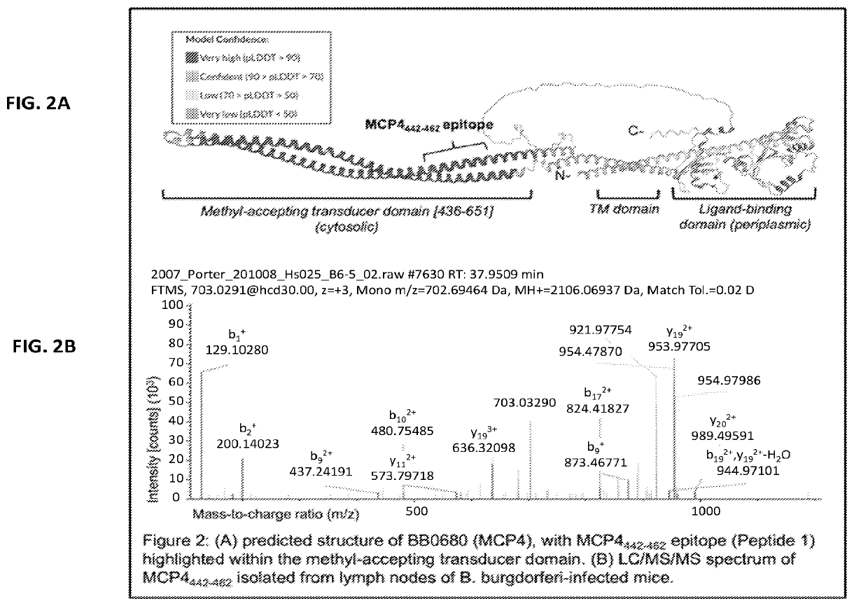

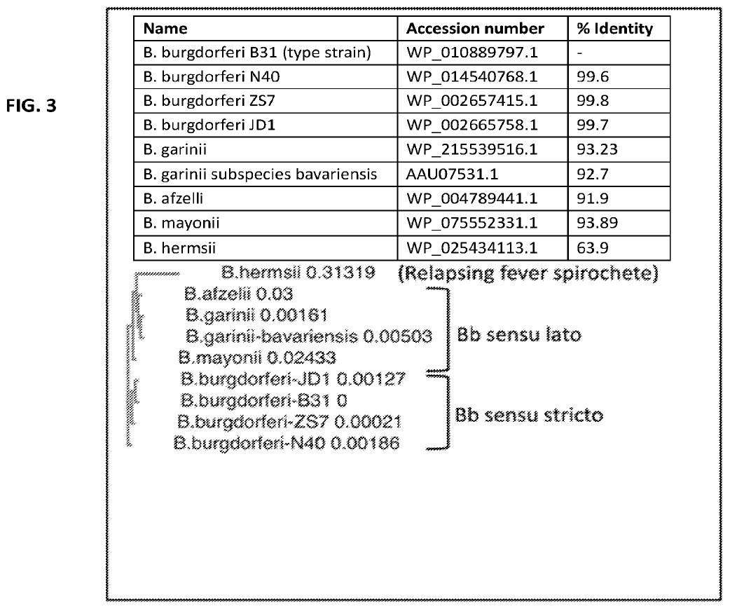

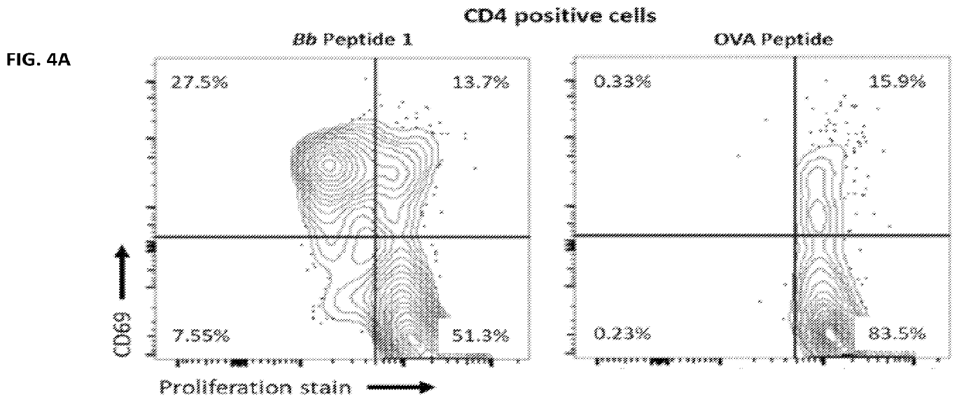

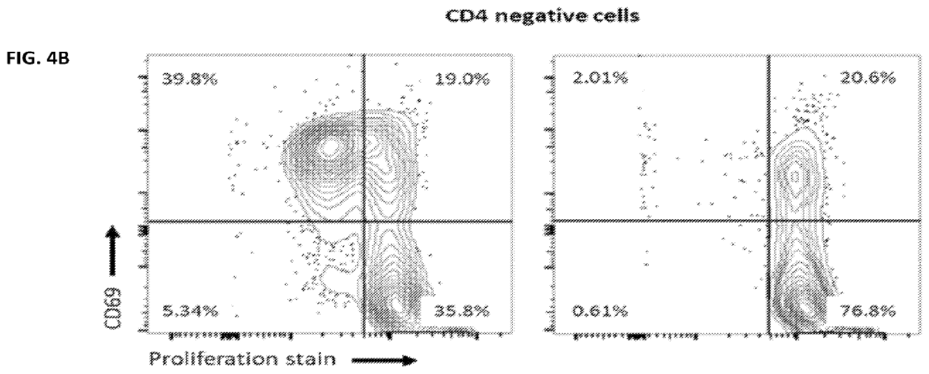

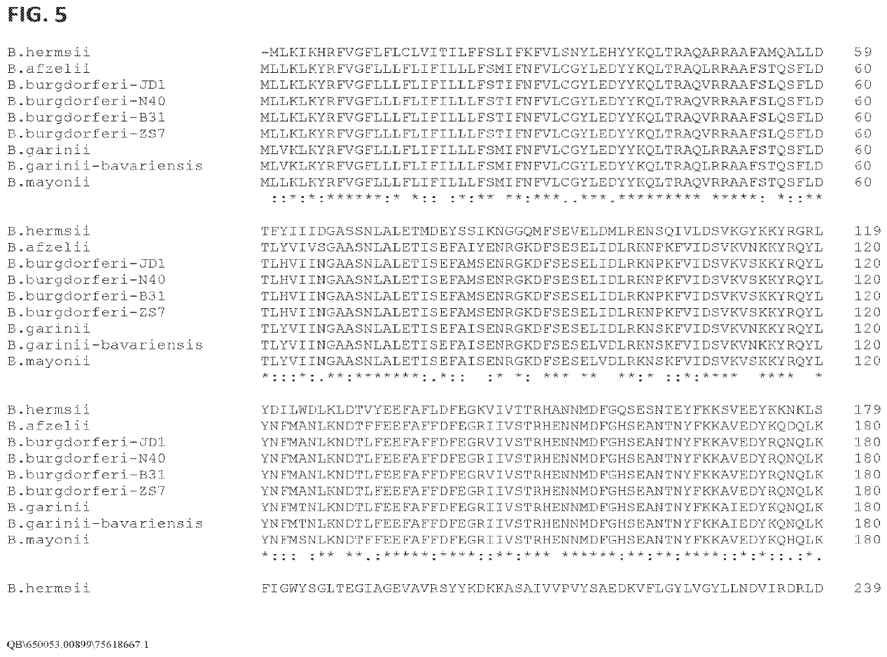

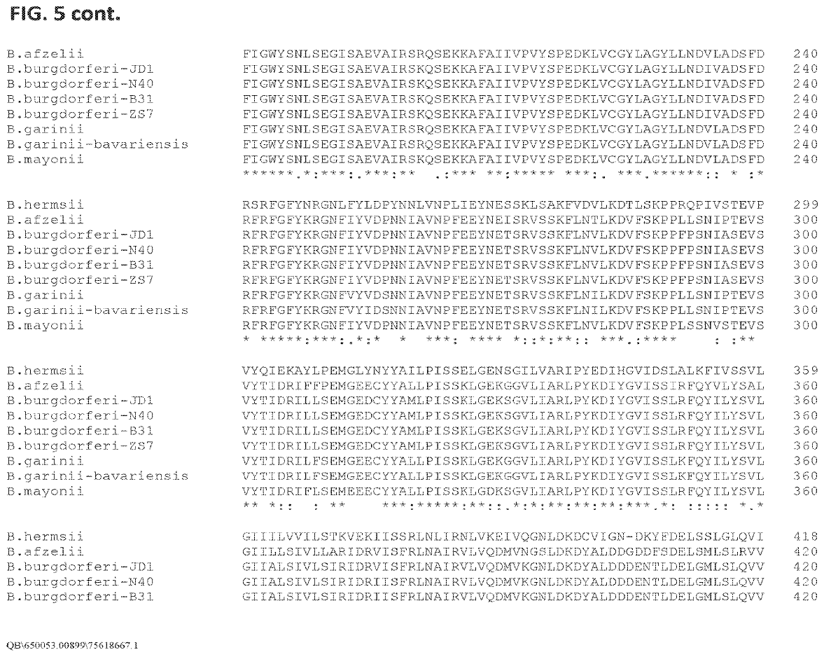

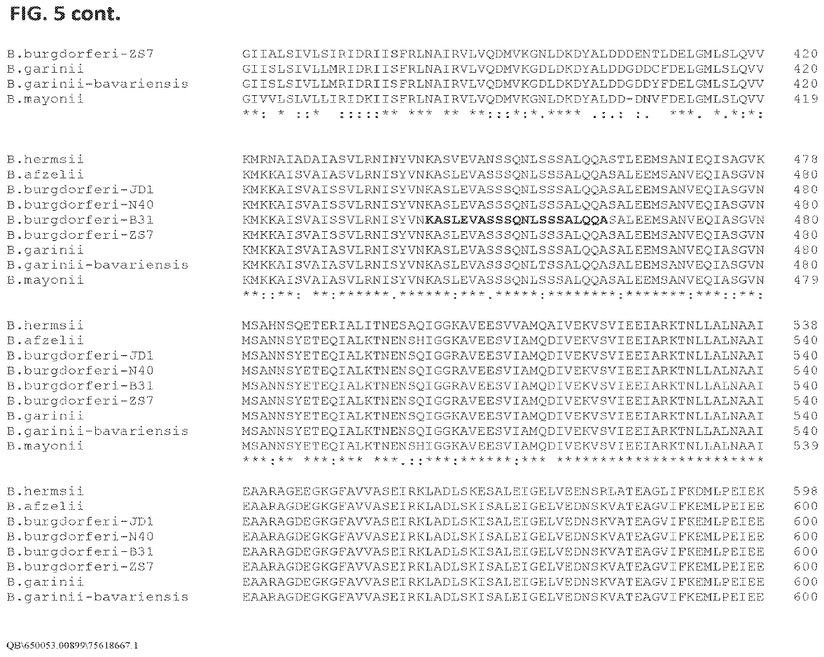

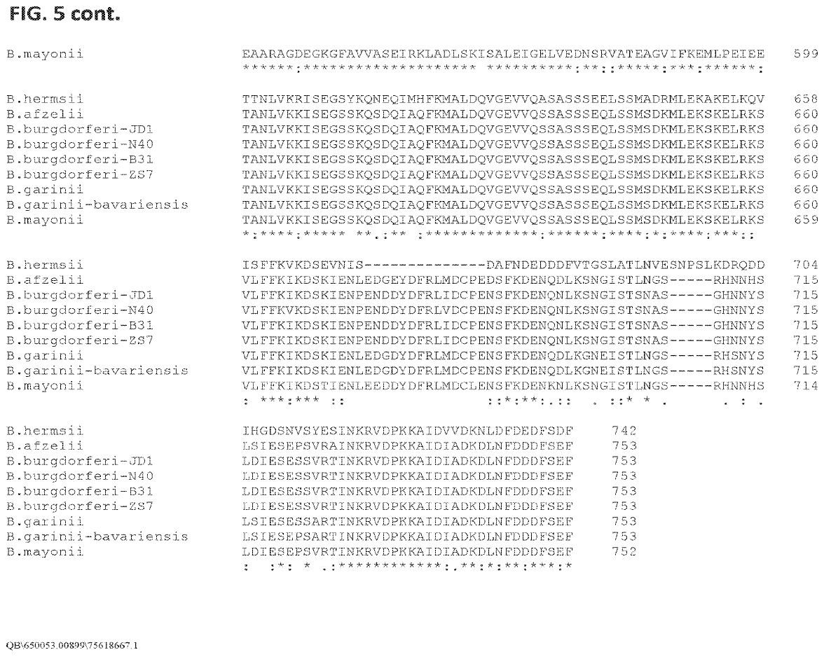

Using an immunopeptidomics approach the inventors screened over 10,000 peptides bound to MHC class II molecules expressed on antigen presenting cells from infected mice (FIG. 1). One peptide was derived from BB0680 (MCP4442-462), hereafter Peptide 1 (SEQ ID NO: 1) (see FIG. 2A). BB0680 is a methyl-accepting chemotaxis protein that is highly conserved amongst both sensu lato and sensu stricto strains of Lyme disease spirochetes (see FIG. 3 and FIG. 5). Peptide 1 is highly immunogenic in mice infected with B. burgdorferi (see FIGS. 4A and 45), validating Peptide 1 as target of CD4+ T cell responses to B. burgdorferi infection. Peptide 1 is one of a number of peptides derived from MCP4 predicted to bind to MHC class II alleles in mice and/or humans (Table 1 below (mouse), Table 2 below (human)). It is anticipated that immunization with Peptide 1 will lead to protective immunity in mice and humans. It is also anticipated that other epitopes derived from MCP4 are immunogenic in mice and humans infected with B. burgdorferi. In some embodiments, and pursuant to the methods disclosed herein, T and B cell reactivity to MCP4 in humans with early and late Lyme disease, and in patients with PTLDS will be measured.

Peptide 1, has the amino acid sequence of SEQ ID NO: 1 and is provided below:

KASLEVASSSQNLSSSALQQA

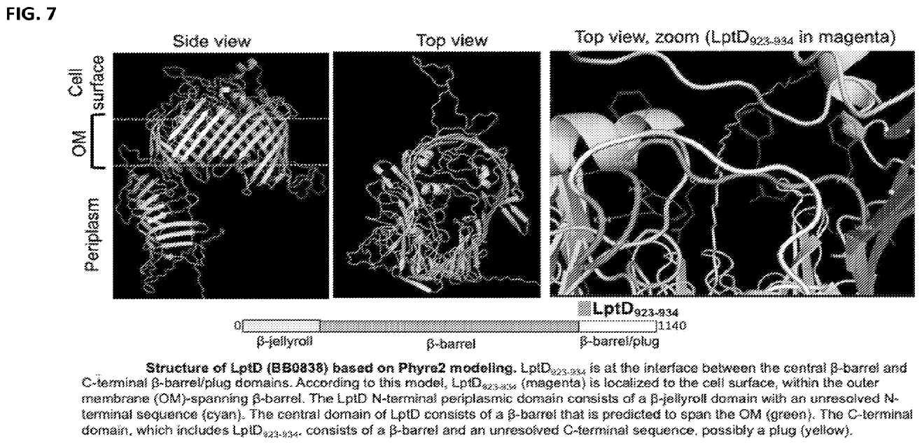

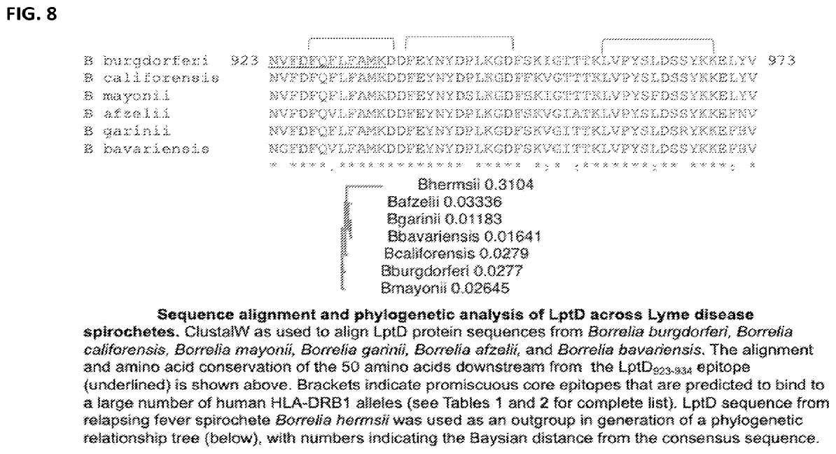

Another peptide identified in the screen was derived from BB0838 (LptD₉₂₃₋₉₃₄), hereinafter Peptide 2 (SEQ ID NO: 2). LptD is an outer membrane protein (FIG. 7) involved in translocation of lipoproteins such as OspA and OspC from the periplasm to the outer membrane and is essential for cell growth. The LptD₉₂₃₋₉₃₄ epitope is located at the interface between the central β-barrel domain and the C-terminal β-barrel/plug domain (FIG. 7). Based on structure modeling, this epitope is localized at or near the cell surface, within the outer membrane-spanning β-barrel (FIG. 7). LptD is highly conserved amongst both sensu lato and sensu stricto strains of Lyme disease spirochetes, and the region surrounding the LptD₉₂₃₋₉₃₄ epitope includes numerous promiscuous epitopes, or peptides predicted to bind to a large number of human HLADRB1 alleles (FIG. 8). Peptide 2 is one of a number of peptides derived from LptD predicted to bind to MHC class II alleles in humans (Table 3 and Table 4 below). It is anticipated that immunization with Peptide 2 will lead to protective immunity in humans and mice. It also anticipated that other epitopes derived from LptD are immunogenic in humans and mice infected with B. burgdorferi. In some embodiments, and pursuant to the methods disclosed herein, T and B cell reactivity to LptD in humans with early and late Lyme disease, and in patients with PTLDS will be measured.

Peptide 2, has the amino acid sequence of SEQ ID NO: 2 and is provided below:

NVFDFQFLFAMK

A peptide from BB0559 (Crr) was also identified in the screen (Crr₁₂₃₋₁₃₈), hereinafter Peptide 3 (SEQ ID NO: 3). Crr is a PTS system glucose-specific component.

Peptide 3, has the amino acid sequence of SEQ ID NO: 3 and is provided below:

HSESVITPVVIANSDE

A peptide from oppAIV was also identified in the screen (oppAIV₁₃₈₋₁₅₂), herein after Peptide 4 (SEQ ID NO: 4). oppAIV is an oligopeptide ABC transporter.

Peptide 4, has the amino acid sequence of SEQ ID NO: 4 and is provided below:

NAEEYFDGKANESE

Additional immunogenic T cell epitopes are identified and described with respect to Table 5 below in the Examples. To date, no other immunogenic T cell epitopes from mice infected with Borrelia burgdorferi have been identified. The only other known Borrelia burgdorferi T cell epitopes target OspA in vaccinated mice. This is of limited value since OspA is typically not expressed by Borrelia burgdorferi during Lyme borreliosis. Furthermore, current vaccine candidates OspA and OspC and diagnostic marker VLSE are poorly conserved between Lyme disease spirochetes, are not expressed during infection (OspA), are rapidly down-regulated following transmission (OspC), or alter their B cell antigenic targets (VLSE). Additionally, all are poor T cell immunogens, which greatly reduces vaccine efficacy.

### DEFINITIONS AND TERMINOLOGY

The disclosed compositions and methods for treating, detecting, and diagnosing infection by a Borrelia sp., such as Borrelia burgdorferi, may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 'B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The terms “identical” or percent “identity,” in the context of two or more nucleic acids or polypeptide sequences, refer to two or more sequences or subsequences that are the same or have a specified percentage of amino acid residues or nucleotides that are the same (i.e., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire nucleic acid or polypeptide sequence or individual portions or domains of a nucleic acid or polypeptide), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence, in the context of nucleic acids. By way of example, in embodiments, the identify exists over a region that is about or at least about 5, 10, 15, 20, 50, 100, or 1000, amino acids in length, to about, less than about, or at least about 220, 100 or 1000 amino acids or nucleotides in length. Optionally, the identity exists over a region that is at least about 5, 10, 15, 20, 21 amino acids in length (e.g., with reference to SEQ ID NO: 1) to about 100, about 20 to about 75, about 30 to about 50 amino acids or nucleotides in length. Non-limiting examples of polypeptide sequences provided herein comprise sequences that are substantially identical to SEQ ID NO: 1, SEQ ID NO: 2, SEQ ID NO: 3, or SEQ ID NO: 4. By way of example, polypeptides that are at least 90%, 91%, 92%, 93%, 94%, 95%, 96%, 97%, 98%, 99% identical to SEQ ID NO: 1, SEQ ID NO: 2, SEQ ID NO: 3, or SEQ ID NO: 4 is provided herein. Polypeptides comprising a difference of 1, 2, 3, 4, 5, 6 or 7, 8, 9 or 0 amino acids as compared to SEQ ID NO: 1, SEQ ID NO: 2, SEQ ID NO: 3, or SEQ ID NO: 4 are also contemplated herein.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

An example of algorithms suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. As will be appreciated by one of skill in the art, the software for performing BLAST analyses is publicly available through the website of the National Center for Biotechnology Information (NCBI). In embodiments, BLAST and BLAST 2.0 are used, with the parameters described herein, to determine percent sequence identity for the nucleic acids and proteins. In embodiments, a BLAST algorithm involves first identifying high scoring sequence pairs (HSPs) by identifying short words of length W in the query sequence, which either match or satisfy some positive-valued threshold score T when aligned with a word of the same length in a database sequence. In embodiments, T is referred to as the neighborhood word score threshold (Altschul et al., supra). In embodiments, these initial neighborhood word hits act as seeds for initiating searches to find longer HSPs containing them. In embodiments, the word hits are extended in both directions along each sequence for as far as the cumulative alignment score can be increased. In embodiments, cumulative scores are calculated using, for nucleotide sequences, the parameters M (reward score for a pair of matching residues; always >0) and N (penalty score for mismatching residues; always <0). In embodiments, for amino acid sequences, a scoring matrix is used to calculate the cumulative score. In embodiments, extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. In embodiments, the BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1, −2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, 7-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions to a peptide, polypeptide, or protein sequence which alters a single amino acid is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Lyme disease” as used herein refers to an infection by a Borrelia bacterium, which is also known as Borreliosis. At least nine species of Borrelia are known to cause Lyme disease, including Borrelia afzelii, Borrelia bissettiae, Borrelia burgdorferi, Borrelia garinii, B. hermsii, Borrelia lusitaniae, Borrelia mayonii, Borrelia spielmanii, and Borrelia valaisiana. Additional Borrelia strains include, without limitation, B. burgdorferi-JD1, B. burgdorferi-N40, B. burgdorferi-B31, B. burgdorferi-ZS7, and B. garinii-bavariensis. Lyme disease is commonly caused by the transfer of Borrelia bacteria to mammalian hosts by certain hard ticks of the Ixodidae family. In the U.S., Lyme disease is most commonly the result of the transfer of Borrelia burgdorferi bacteria by the blacklegged tick, also known as a deer tick (Ixodes scapularis or Ixodes pacificus). Outside the U.S., Ixodes ricinus and Ixodes persulcatus act as Borreliosis vectors. Without early detection and treatment, the bacteria travel through the bloodstream and affect various tissues in the host. The infection may develop into an inflammatory condition that affects multiple systems, starting with skin, joints, and nervous system and moving to organs. Symptoms can include one or more of fever, chills, headache, fatigue, muscle pain, joint pain, swollen lymph nodes, a bullseye rash, termed erythema migrans, facial palsy, and arthritis.

The term “subject” may be used interchangeably with the terms “individual” and “patient” and includes human and non-human subjects. In some embodiments, subjects may be any animal that can be infected with a bacterium, such as Borrelia burgdorferi. In some embodiments, the subject is a mammal, such as a human, dog, cat, or livestock, such as cattle, pigs, or sheep. In some embodiments, a subject may include a wild, domesticated, or captive population of animals such as deer, elk, bison, etc. In some embodiments, a subject may include one or more wild or domesticated bird species, such as chickens, ducks, or turkeys.

In some embodiments, the methods include detecting the presence of Borrelia burgdorferi in a subject. In some embodiments, the methods include detecting the presence of Borrelia afzelii, Borrelia burgdorferi, Borrelia garinii, or Borrelia mayonii in a subject.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

A non-limiting example of a treatment for a subject diagnosed with Lyme disease includes, but is not limited to, administration of a composition comprising a polypeptide of SEQ ID NO: 1, a polypeptide at least 90% identical to SEQ ID NO: 1, a polypeptide of SEQ ID NO: 2, or a polypeptide at least 90% identical to SEQ ID NO: 2, or a combination thereof. In some embodiments, the composition comprises an adjuvant. In some embodiments, the composition comprises a vaccine.

A non-limiting example of a treatment for infection by Borrelia sp. including, but not limited to B. burgdorferi includes, but is not limited to, administration of a composition comprising a polypeptide of SEQ ID NO: 1, a polypeptide at least 90% identical to SEQ ID NO: 1, a polypeptide of SEQ ID NO: 2, or a polypeptide at least 90% identical to SEQ ID NO: 2, or a combination thereof. In some embodiments, the composition comprises an adjuvant. In some embodiments, the composition comprises a vaccine.

A non-limiting example of a preventative treatment for a subject at risk of exposure to a tick causing Lyme disease includes, but is not limited to, administration of a composition comprising a polypeptide of SEQ ID NO: 1, a polypeptide at least 90% identical to SEQ ID NO: 1, a polypeptide of SEQ ID NO: 2, or a polypeptide at least 90% identical to SEQ ID NO: 2, or a combination thereof. In some embodiments, the composition comprises an adjuvant. In some embodiments, the composition comprises a vaccine.

A non-limiting example of a preventative treatment for a subject at risk for infection by Borrelia sp. including, but not limited to B. burgdorferi includes, but is not limited to, administration of a composition comprising a polypeptide of SEQ ID NO: 1, a polypeptide at least 90% identical to SEQ ID NO: 1, a polypeptide of SEQ ID NO: 2, or a polypeptide at least 90% identical to SEQ ID NO: 2, or a combination thereof. In some embodiments, the composition comprises an adjuvant. In some embodiments, the composition comprises a vaccine.

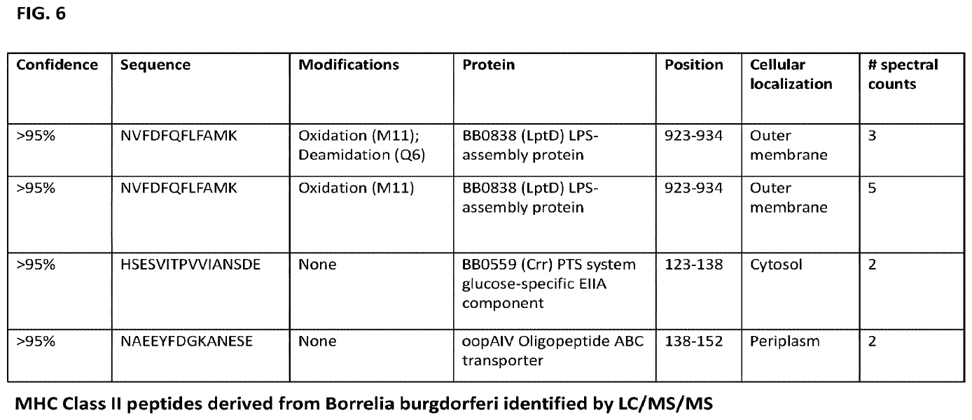

In some embodiments, a non-limiting treatment or preventative treatment includes vaccination of a subject in need thereof (e.g., a subject diagnosed with Lyme disease, or a subject at risk of exposure to Lyme disease), with a composition comprising one or more of the epitopes presented in the tables provided in FIGS. 5 and 6.

As used herein, the term “adjuvant” refers to ingredients used in some vaccines to create a stronger immune response in subjects receiving the vaccine. By way of example, but not by way of limitation, adjuvants include aluminum, monophosphoryl lipid A (MPL), MPL+aluminum salt, oil in water emulsion, e.g., composed of squalene, MPL and QS-21, cytosine phosphaguanine (CpG).

As used herein, the term “antibody,” or “antibody molecule” (used synonymously herein) refer to naturally occurring immunoglobulin molecules with varying structures. Typically, antibodies are gamma globulin proteins that can be found in blood or other bodily fluids of vertebrates and are used by the immune system to identify foreign materials, such as bacteria, viruses, and toxins. Antibodies bind, by non-covalent interactions, with high affinity to other molecules or structures known as antigens. This binding is specific in the sense that an antibody molecule will only bind to a specific structure with high affinity. The unique part of the antigen recognized by an antibody molecule is called an epitope, or antigenic determinant. The part of the antibody molecule binding to the epitope is sometimes called paratope and resides in the so-called variable domain, or variable region (Fv) of the antibody. The variable domain comprises three complementary-determining regions (CDR's) spaced apart by framework regions (FR's). There are at least five major classes of antibodies: IgA, IgD, IgE, IgG, and IgM, and several of these may be further divided into subclasses (isotypes), e.g., IgG1, IgG2, IgG3, IgG4, IgAQ1, and IgA2. They are typically made of basic structural units—each with two large heavy chains and two small light chains—to form, for example, monomers with one unit, dimers with two units or pentamers with five units. Non-limiting examples of antibodies include any known class and/or isotype, such as, e.g., IgA (e.g. IgA1, IgA2, and sIgA), IgD, IgE, IgG (e.g. IgG1, IgG2, IgG3, or IgG4), and IgM. The “class” of an antibody may refer to the type of constant domain or constant region possessed by its heavy chain. For example, heavy chain constant domains may be used to classify different classes of immunoglobulins, such as, e.g., α, γ, δ, ε, and μ. In some embodiments, the antibody is a native human IgG. IgG antibodies are typically heterotetrameric glycoproteins of about 150,000 daltons, composed of two identical light chains and two identical heavy chains that are disulfide bonded. The light chain of an antibody may be assigned to certain types, e.g. kappa and lambda, based on the amino acid sequence of its constant domain.

The terms “epitope” and “antigenic determinant”, which can be used interchangeably, refer to the part of a macromolecule, such as a polypeptide, carbohydrate, or a lipid, that is recognized by antigen-binding molecules, such antibodies. Epitopes define the minimum binding site for an antibody molecule, and thus represent the target of specificity of an antibody molecule. In some embodiments, an epitope comprises, or consists of SEQ ID NO: 1, or a polypeptide at least 90% identical to SEQ ID NO: 1, or an epitope provided in Tables 1 and 2. In the same or alternative embodiments, an epitope comprises, or consists of SEQ ID NO: 2, or a polypeptide at least 90% identical to SEQ ID NO: 2, or an epitope provided in Tables 3 and 4. In various embodiments, an epitope comprises or consists of a polypeptide provided in Table 5, or a polypeptide at least 90% identical to one or more of the polypeptides provided in Table 5.

An antibody molecule that can “bind”, “bind to”, “specifically bind”, or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein.

Generally, the term “specificity” refers to the number of different types of antigens or epitopes to which a particular antigen-binding molecule or antigen-binding protein (such as an immunoglobulin, an antibody, an immunoglobulin single variable domain) can bind. The specificity of an antigen-binding protein can be determined based on its affinity and/or avidity. The affinity, represented by the equilibrium constant for the dissociation of an antigen with an antigen-binding protein (K_{D}), is a measure for the binding strength between an epitope and an antigen-binding site on the antigen-binding protein: the lesser the value of the Ko, the stronger the binding strength between an epitope and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as an antibody of the invention) and the pertinent antigen. Avidity is related to both the affinity between an epitope and its antigen binding site on the antigen-binding molecule and the number of pertinent binding sites present on the antigen-binding molecule.

As used herein, the term “panel” refers to a collection of two or more biomolecules (e.g. antigens) wherein the two or more biomolecules are organized such that the two or more biomolecules can be differentiated from each other in a detecting step. This organization can be a spatial arrangement (e.g. biomolecule 1 is in section 1 of the panel, biomolecule 2 is in section 2 of the panel, etc.) or a difference in a detection aspect, such as each biomolecule can be differentiated by a different detectable agent (e.g. biomolecule 1 can be detected with color 1, biomolecule 2 can be detected with color 2, etc.).

As used herein, the term “solid support” refers to any substrate or support matrix to which a molecule (such as a polypeptide, antibody, or antibody-polypeptide complex) can be bound, either reversibly or irreversibly. Non-limiting examples of solid supports include, without limitation, a bead, plate, slide, flow chamber, chip, cartridge, flow cell, etc. The solid support may formed from glass, ceramic, polymers (e.g., plastic, latex, polystyrene, polyacrylamide, polyvinylchloride, polypropylene, polyethylene, polylactic acid), cellulose (e.g., paper). In some embodiments, a polypeptide is immobilized on a solid support. In some embodiments, the polypeptide is SEQ ID NO: 1. In certain embodiments, the polypeptide is SEQ ID NO: 2. In some embodiments, the immobilized polypeptide is bound to the solid support, either reversibly or irreversibly, to form a platform for an antibody binding reaction. In some embodiments, there are two or more polypeptide which are immobilized on the solid support in configuration so as to form a polypeptide panel.

As used herein, the term “bead” refers to a microbead or relatively small bead having a diameter less than about 500 μm, typically with a diameter of less than about 100 μm, 50 μm, or 10 μm. While a bead may vary in shape, typically a bead is substantially spherical, e.g. a microsphere. Microbeads are known in the art and can comprise various materials such as, e.g., latex, polystyrene, polyacrylamide, polyvinylchloride, polypropylene, polyethylene, polylactic acid, ceramic, glass, and magnetic compositions.

As used herein, the term “plate” encompasses a solid support comprising a variety of types, including plastic or glass plates. In some embodiments, the plate is a 96-well, 384-well, or 1536-well plastic plate.

As used herein, a “detectable binding agent” refers to a molecule that binds, either directly or indirectly, to a target molecule, and can be detected either directly, or with further chemical or enzymatic reaction. By way of example, but not by way of limitations, a detectable binding agent may comprise an antibody linked to an enzyme, such as horseradish peroxidase (HRP). Addition of an HRP substrate, under enzymatic reaction conditions, will allow detection of the HRP-bound molecule and the target. Other examples of detectable binding agents and include antibodies or MHC class II tetramer reagents (tetramers) linked to detectable labels, such as fluorescent labels. Such constructs are well known in the art and are not intended to limit the scope of the present methods and compositions.

As used herein, a “detectable agent” or “detectable label” refers to an agent or label that can be detected either directly, or with further chemical or enzymatic reaction. By way of example, but not by way of limitations, a detectable agent or label may comprise a fluorescent moiety or an enzyme, such as HRP. Other examples of detectable agents and labels include radioisotope. Such agents and labels are well known in the art and are not intended to limit the scope of the present methods and compositions.

As used herein, the term “buffer” refers to a pH buffering agent that helps maintain the pH of an aqueous solution. Non-limiting examples of pH buffering agents include phosphate buffers (e.g. PBS), Tris, citric acid, histidine, glycine, HEPES, MOPS, and PIPES.

As used herein, the term “stabilizer” refers to an agent that stabilizes a component of a composition, such as a protein, antibody, and/or polypeptide, thereby helping to preserve its stability and integrity. Non-limiting examples of stabilizers include glycerol, polysorbates, polyethylene glycols (PEGs), glycine, histidine, arginine, sugars (e.g. trehalose), and polyols (e.g., mannitol, sorbitol and glycerol).

As used herein, the term “non-specific binding surface blocking agent” refers to an agent that reduces the non-specific binding between antibodies and other molecules. Non-limiting examples of non-specific binding surface blocking agents include milk proteins (e.g. casein) and bovine serum albumin (BSA).

As used herein, the term “macromolecular crowding agent” refers to an inert agent that excludes volume in solution from other macromolecules, such as proteins and antibodies. Non-limiting examples of macromolecular crowding agents include polyethylene glycols (PEGs), dextran, and high molecular weight, highly branched polysaccharides.

As used herein, the term “antioxidant” refers to an agent that inhibits oxidation reactions. Non-limiting examples of antioxidants include citrate, EDTA (ethylenediaminetetraacetic acid), methionine, ascorbic acid, and thiols.

As used herein, the term “preservative” refers to an agent that prevents or inhibits microbiological growth. Non-limiting examples of preservatives include CMIT (5-chloro-2-methylisothiazol-3(2H)-one), MIT (2-methylisothiazol-3(2H)-one), and sodium azide.

As used herein, the term “surfactant” refers to an agent that modulates the solubility of another molecule or agent in a solution. Non-limiting examples of surfactants include polysorbate 20, polysorbate 80, Triton X-100, and pluronic F68.

As used herein, the term “cut-off value” refers to a dividing point for a quantitative result of a diagnostic test or screen to be indicative of a positive or negative result based on measurements of the level of a molecular marker(s). This disclosure provides methods of detecting Lyme disease and/or Borrelia infection in a subject comprising the step of comparing a measured level of molecular marker to a predetermined value. In some embodiments, comparing a measured value of an antibody against an epitope (e.g., SEQ ID NO: 1 and/or SEQ ID NO: 2) to a predetermined cut-off value of the antibody may be used to decide whether the method indicates a positive or negative result of Lyme disease and/or Borrelia infection in the subject. As used herein, a “cut-off value” refers to a value above which a measured epitope antibody level in a sample is indicative of the positive presence of a Borrelia infection and/or Lyme disease in the subject from which the sample was derived.

As used herein the term “standard two-tier test” (SST) refers to a decision tree that describes the steps required to currently test for Lyme disease. The first required test is the Enzyme Immunoassay (EIA) or Immunofluorescence Assay (IFA). If this test yields negative results, the provider should consider an alternative diagnosis; or in cases where the patient has had symptoms for less than or equal to 30 days, the provider may treat the patient and follow up with a convalescent serum. If the first test yields positive or equivocal results, two options are available: 1) If the patient has had symptoms for less than or equal to 30 days, an IgM Western Blot is performed; 2) if the patient has had symptoms for more than 30 days, the IgG Western Blot is performed. The IgM should not be used if the patient has been ill for more than 30 days.

The STT has several well-known shortcomings. Two of the most notable include low sensitivity and specificity in early infection, and the inability to determine infection status after antibody therapy. Diagnostic compositions and methods disclosed herein e.g., that incorporate Peptide 1 (SEQ ID NO: 1) and/or Peptide 2 (SEQ ID NO: 2), such as ELISpot assays, MCH tetramer assays, and proliferation assays, overcome these limitations.

### Compositions

In certain embodiments, T cell immunogens derived from BB0680/MCP4 may be used for novel Lyme disease vaccine targets, either alone or in combination with B cell antigens such as OspA or OspC. Thus, disclosed herein are compositions comprising an epitope of the BB0680/MCP4 peptide, wherein the epitope comprises or consists of SEQ ID NO: 1. In some embodiments, the composition comprises one or more additional antigens, such as outer surface protein A, and outer surface protein C (OspA and OspC, respectively). In some embodiments, the additional antigen is a naturally occurring antigen; in some embodiments, the antigen is recombinant.

```
OspA sequence is shown below (NCBI: Ref. Seq: WP_075552755.1).
(SEQ ID NO: 5)
    1 mkkyllgigl ilaliackqn vssldeknsv svdlpgeikv lvskekdkdg kyslmatvdk
  61 lelkgtsdkn ngsgvlegvk adkskvkltv sddlskttle vlkedgktlv srkvtskdks
121 steekfnekg elaektmtra detrleytei ksdgsgkake vlkgyalegt ltaekttlvv
181 kegtvtlskn isksgevtae Indtdsaaat kktgawnsgt stltitansk ktkdlvftke
241 ntitvqkydt agiklegsav eiktldelkn alk
OspC sequence is shown below (GenBank: AA88006.1)
(SEQ ID NO: 6)
    1 ilmtlflfis cnnsgkdgnt sansadesvk gpnlteiskk itdsnavlla vkeveallss
  61 idelakaigk kikndgslgd eanhneslla gaytistlit qklsklngse glkekiaaak
121 kcseefstkl kdnhaqlgiq gvtdenakka ilkanaagkd kgveelekls gsleslskaa
181 kemlansvke ltspvvaes
```

In various embodiments, the T cell immunogens derived from BB0838/LptD may be used for novel Lyme disease vaccine targets, either alone or in combination with B cell antigens such as OspA or OspC. Thus, disclosed herein are compositions comprising an epitope of the BB0838/LptD peptide. In certain embodiments, the compositions disclosed herein comprise an epitope of the BB0838/LptD peptide, wherein the epitope comprises or consists of SEQ ID NO: 2. In some embodiments, the composition comprises one or more additional antigens, such as outer surface protein A, and outer surface protein C (OspA and OspC, respectively). In some embodiments, the additional antigen is a naturally occurring antigen; in some embodiments, the antigen is recombinant.

In one or more embodiments, the compositions disclosed herein can include an epitope of the BB0838/LptD peptide and an epitope of the BB0680/MCP4 peptide. For instance, in certain embodiments, the compositions disclosed herein can include an epitope of the BB0838/LptD peptide, wherein the epitope comprises or consists of SEQ ID NO: 2, and an epitope of the BB0680/MCP4 peptide, wherein the epitope comprises or consists of SEQ ID NO: 1. In various embodiments, the compositions may also include one or more additional antigens, such as outer surface protein A, and outer surface protein C (OspA and OspC, respectively). In some embodiments, the additional antigen is a naturally occurring antigen; in some embodiments, the antigen is recombinant.

In certain embodiments, the compositions disclosed herein can include an epitope of a BB0559/Crr peptide, e.g., Peptide 3, SEQ ID NO: 3, and/or an epitope of oppAIV, e.g., Peptide 4, SEQ ID NO: 4.

In some embodiments, the compositions disclosed herein comprise an adjuvant. In some embodiments, the compositions disclosed herein are provided in lyophilized form.

### Methods

Disclosed herein are methods for the treating and diagnosis/detection of infection with Borrelia sp.

In some embodiments, the methods comprise contacting an antibody-containing sample from a subject with a polypeptide of SEQ ID NO: 1 and/or with a polypeptide of SEQ ID NO: 2.

In some embodiments, the method includes allowing the binding of antibodies in the sample to the polypeptide; contacting the bound antibodies with a detectable binding agent; detecting the detectable binding agent; measuring the level of antibodies bound to the polypeptide; and comparing the measured levels of bound antibody to a value, such as a predetermined cutoff value or a value calculated based on the level of a maker in the sample measured in parallel or a control sample (e.g. a sample from a Borrelia sp. exposure naïve subject).

The level of antibodies bound to the polypeptide can then be compared to a value to inform a diagnosis or treatment decision, such as, a diagnosis of Lyme disease or a Borrelia infection. The level of antibodies bound to the polypeptide can be used to monitor the progression of a treatment for Lyme disease or a Borrelia infection. The level of antibodies bound to the polypeptide, or after a series of measurements over a duration of weeks or months, can be used to estimate when the subject was exposed to Borrelia sp.

In some embodiments, if the measured level is greater than a predetermined antibody value for the polypeptide, and the subject is diagnosed as positive for Lyme disease. In some embodiments, if the measured level is less than a predetermined antibody value for the polypeptide, the subject is diagnosed as negative for Lyme disease.

In some embodiments, the method further comprises treating the subject for Lyme disease.

As used herein the terms “predetermined value” and “control value” are used interchangeably. For example, in some embodiments, the predetermined value or control value is a cutoff value based on a naïve control antibody level, either measured independently or measured using the same sample. A suitable cutoff value can be determined using routine techniques known to the skilled worker. In some embodiments, the cutoff value is calculated from a measured naïve control level adjusted by a standard deviation or parameter based on the standard deviation. For example, a cutoff value can be a mean naïve control value+2 standard deviations. In some embodiments, a subject is tested multiple times, and the control value (or predetermined value) comprises the test value of a previous sample from the same subject (e.g., to determine whether a subject is responding favorably to a treatment). In such an exemplary embodiment, the control value (the predetermined value) is greater than the test value if the subject is responsive to treatment, or the control value (the predetermined value) is equal to or less than the test value if the subject is non-responsive to treatment. While it is understood that in some situations a control value is not a predetermined value per-se, the terms “predetermined value” and “control value” are intended to encompass such situations. For example, if the control sample, (e.g., a known naïve control sample, or a known positive sample) is tested simultaneously with the unknown, test sample, and the control value is determined at the time of test completion, then the “control value” is not necessarily “predetermined.” However, it is known in advance of testing that the control sample will have a value indicative of a predetermined infection status (e.g., naïve or positive).

In some embodiments, in addition to SEQ ID NO: 1 and/or SEQ ID NO: 2, multiple different polypeptides are employed in testing. In some embodiments, the one or more polypeptides can be configured on a solid support.

In some embodiments of any of the methods above, the detecting comprises detecting the bound antibodies or antibody-epitope complexes using a detectable binding agent.

In some embodiments of any of the methods above, the sample comprises or consists of a blood sample (e.g. whole blood), plasma sample, and/or serum sample.

In some embodiments of any of the methods above, the detecting step comprises immobilizing antibody-epitope complexes to a solid support.

In some embodiments, the solid support comprises or consists of a plate, bead, flow cell, flow chamber, microfluidic chamber, and/or microchip comprising a plurality of microfluidic chambers.

In some embodiments, the detecting step comprises flowing the reagent-composition through a flow-chamber. In some embodiments, the detection comprises a microfluidic chamber, microchip comprising a plurality of microfluidic chambers, or lateral flow assay.

In some embodiments, the subject is human and the detectable binding agent comprises an anti-human IgG comprising a detectable label or conjugated to a detectable agent. In some embodiments, the detectable agent comprises an enzyme, such as a horseradish peroxidase.

In some embodiments, a T cell assay is provided. In some embodiments, the T cell assay is configured as an ELISpot assay, an MHC tetramer assay, or a cell proliferation assay.

As used herein, the term “ELISpot” assay refers to the “enzyme-linked immune absorbent spot” assay. The ELISpot assay is often used to quantitatively measure the frequency of cytokine secretion for a single cell. The ELISpot Assay is also a form of immunostaining since it is classified as a technique that uses antibodies to detect a protein analyte, with the word analyte referring to any biological or chemical substance being identified or measured.

As used herein an “MHC tetramer assay” refers to an assay method that uses tetrameric proteins to detect and quantify T cells that are specific for a given antigen within a blood sample. The tetramers used in the assay are made up of four major histocompatibility complex (MHC) molecules, which are found on the surface of most cells in the body. MHC molecules present peptides to T-cells as a way to communicate the presence of viruses, bacteria, cancerous mutations, or other antigens in a cell. If a T-cell's receptor matches the peptide being presented by an MHC molecule, an immune response is triggered. Thus, MHC tetramers that are bioengineered to present a specific peptide can be used to find T-cells with receptors that match that peptide. The tetramers are labeled with a detectable label, such as a fluorophore, allowing tetramer-bound T-cells to be analyzed with flow cytometry. Quantification and sorting of T-cells by flow cytometry enables researchers to investigate immune response to viral infection and vaccine administration as well as functionality of antigen-specific T-cells. Generally, if a subject's immune system has encountered a pathogen, the subject will possess T cells with specificity toward some peptide on that pathogen. Hence, if a tetramer stain specific for a pathogenic peptide results in a positive signal, this may indicate that the subject's immune system has encountered and built a response to that pathogen.

T cell proliferation assays are well known in the art and are often used in conjunction with flow cytometric analysis, e.g., to characterize T cells.

In some embodiments of any of the methods above, the subject previously tested positive for Lyme disease by standard two-tier (STT) testing.

In some embodiments of any of the methods above, the subject exhibits one or more of the following symptoms: erythema migrans, facial palsy, and arthritis; and/or the subject already tested positive for Lyme disease in standard two-tier test (STT). In some embodiments, the subject has exhibited the symptom for 3 months or less, 2 months or less, 1 month or less, 2 weeks or less, 1 week or less, 3 days or less, or 1 day or less.

In some embodiments of any of the methods above, the subject has not previously been treated for Lyme disease.

In some embodiments of any of the methods above, the subject is asymptomatic for Lyme disease, but may have come in contact with a blacklegged tick selected from Ixodes scapularis, Ixodes pacificus, Ixodes ricinus, and Ixodes persulcatus. In some embodiments, the possible contact between the subject and the tick was within 3 months, within 2 months, within 1 month, within 2 weeks, within 1 week, within 3 days, or within 1 day.

In some embodiments of any of the methods above, the subject is asymptomatic for Lyme disease and has previously been treated for Lyme disease. In some embodiments, the subject is asymptomatic for Lyme disease and has not previously been treated for Lyme disease.

In some embodiments of any of the methods above, the subject has previously been treated for Lyme disease and exhibits one or more of the following symptoms: erythema migrans, facial palsy, and arthritis; and/or the subject previously tested positive for Lyme disease in standard two-tier test (STT). In some embodiments, the subject has not received treatment for Lyme disease for at least 1 year, for at least 5 years, for at least 10 years, or for at least 15 years.

In some embodiments of any of the methods above, the method comprises a step of treating the subject for Lyme disease.

In some embodiments of any of the methods above, the subject is being treated for Lyme disease. In some further embodiments, the method is used to monitor the progression or the efficacy of the treatment.

### Kits

Disclosed herein are kits for the detection of infection with Borrelia sp. In some embodiments, the kit comprises a composition described herein and one additional reagent or device. For example, in some embodiments, the kits include a polypeptide comprising SEQ ID NO: 1, or a polypeptide at least 90% identical to SEQ ID NO: 1, configured on a solid support or in a reagent composition. In certain embodiments, the kits include a polypeptide comprising SEQ ID NO: 2, or a polypeptide at least 90% identical to SEQ ID NO: 2, configured on a solid support or in a reagent composition. In some embodiments, the kits include a polypeptide comprising SEQ ID NO: 1 and/or SEQ ID NO: 2. In one or more embodiments, the kits include a polypeptide at least 90% identical to SEQ ID NO: 1 and/or a polypeptide at least 90% identical to SEQ ID NO: 2. In some embodiments, the additional reagent or device is a flow chamber, bead composition, ELISpot assay reagent and/or platform, ELISA reagent, anti-human IgG antibody, and/or a detectable reagent such as a HRP substrate. In some embodiments, a kit includes reagents and components for an MHC tetramer assay. In some embodiments, the kit comprises instructions for using the components of the kit in the performance of a method described herein. In some embodiments, the kits comprise instructions, and are configured for in-home use.

By way of example but not by way of limitation, the kits and components there may be used to evaluate T and/or B cell responses to the epitope(s), which provide novel diagnostic biomarkers for active infection of B. burgdorferi or post-treatment Lyme disease syndrome (PTLDS). In some embodiments, the kits are configured for ELISpot assays, MCH tetramer assays, and/or T cell proliferation assays, and include SEQ ID NO: 1 and/or SEQ ID NO: 2.

Table 1 below shows the predicted mouse MHCII binding of peptides derived from BB0680 determined using NetMHCIIpan (V 4.0). The prediction outcome for each molecule includes the following columns in the table: Pos Residue number (starting from 0); MHC MHC molecule name; Peptide Amino acid sequence; Of Starting position offset of the optimal binding core (starting from 0); Core Binding core register; Core_Rel Reliability of the binding core, expressed as the fraction of networks in the ensemble selecting the optimal core; Score_EL Eluted ligand prediction score; % Rank_EL Percentile rank of eluted ligand prediction score; BindLevel (SB: strong binder, WB: weak binder). The peptide will be identified as a strong binder if the % Rank is below the 1% threshold for the strong binders. The peptide will be identified as a weak binder if the % Rank is above the 1% threshold of the strong binders but below the 5% threshold for the weak binders.

TABLE 1
Predicted mouse MHC II binding peptides derived from BB0680 determined
using NetMHCIIpan (V.4.0)

| | MHC | SEQ ID | | | SEQ ID | | | | % | |
|---|---|---|---|---|---|---|---|---|---|---|
| Pos | allele | NO: | Peptide | Of | NO: | Core | Core_Rel | Score_EL | Rank_EL | BindLevel |
| 37 | H-2- | 27 | EDYYKQLTR | 3 | 115 | YKQLTR | 0.793 | 0.343513 | 0.39 | <=SB |
| | IEk | | AQVRRA | | | AQV | | | | |
| 38 | H-2- | 28 | DYYKQLTRA | 2 | 115 | YKQLTR | 0.6 | 0.146296 | 0.66 | <=SB |
| | IEd | | QVRRAA | | | AQV | | | | |
| 39 | H-2- | 29 | YYKQLTRAQ | 4 | 116 | LTRAQV | 0.507 | 0.06832 | 2.8 | <=WB |
| | IEd | | VRRAAF | | | RRA | | | | |
| 39 | H-2- | 29 | YYKQLTRAQ | 4 | 116 | LTRAQV | 0.96 | 0.574296 | 0.24 | <=SB |
| | IAu | | VRRAAF | | | RRA | | | | |
| 40 | H-2- | 30 | YKQLTRAQV | 3 | 116 | LTRAQV | 0.78 | 0.253482 | 1.46 | <=WB |
| | IEk | | RRAAFS | | | RRA | | | | |
| 40 | H-2- | 30 | YKQLTRAQV | 3 | 116 | LTRAQV | 1 | 0.622742 | 0.89 | <=SB |
| | IAd | | RRAAFS | | | RRA | | | | |
| 40 | H-2- | 30 | YKQLTRAQV | 3 | 116 | LTRAQV | 0.993 | 0.220788 | 3.69 | <=WB |
| | IAb | | RRAAFS | | | RRA | | | | |
| 59 | H-2- | 31 | LDTLHVIING | 3 | 117 | LHVIIN | 0.753 | 0.179169 | 4.26 | <=WB |
| | IEk | | AASNL | | | GAA | | | | |
| 62 | H-2- | 32 | LHVIINGAAS | 4 | 118 | INGAAS | 0.933 | 0.498855 | 0.54 | <=SB |
| | IAu | | NLALE | | | NLA | | | | |
| 63 | H-2- | 33 | HVIINGAASN | 3 | 118 | INGAAS | 1 | 0.530619 | 1.05 | <=WB |
| | IAb | | LALET | | | NLA | | | | |
| 63 | H-2- | 33 | HVIINGAASN | 3 | 118 | INGAAS | 0.947 | 0.335717 | 4.61 | <=WB |
| | IAd | | LALET | | | NLA | | | | |
| 78 | H-2- | 34 | ISEFAMSENR | 3 | 119 | FAMSEN | 0.973 | 0.167466 | 0.48 | <=SB |
| | IEd | | GKDFS | | | RGK | | | | |
| 93 | H-2- | 35 | ESELIDLRKN | 3 | 120 | LIDLRK | 0.693 | 0.044345 | 4.95 | <=WB |
| | IEd | | PKFVI | | | NPK | | | | |
| 101 | H-2- | 36 | KNPKFVIDS | 4 | 121 | FVIDSV | 0.773 | 0.379345 | 0.21 | <=SB |
| | IEk | | VKVSKK | | | KVS | | | | |
| 102 | H-2- | 37 | NPKFVIDSV | 3 | 121 | FVIDSV | 0.747 | 0.263283 | 4.67 | <=WB |
| | IAu | | KVSKKY | | | KVS | | | | |
| 103 | H-2- | 38 | PKFVIDSVK | 2 | 121 | FVIDSV | 0.64 | 0.224043 | 0.22 | <=SB |
| | IEd | | VSKKYR | | | KVS | | | | |
| 104 | H-2- | 39 | KFVIDSVKV | 3 | 122 | IDSVKV | 0.667 | 0.152686 | 0.59 | <=SB |
| | IEd | | SKKYRQ | | | SKK | | | | |
| 104 | H-2- | 39 | KFVIDSVKV | 3 | 122 | IDSVKV | 0.64 | 0.333347 | 0.45 | <=SB |
| | IEk | | SKKYRQ | | | SKK | | | | |
| 105 | H-2- | 40 | FVIDSVKVS | 5 | 123 | VKVSK | 0.673 | 0.087195 | 1.88 | <=WB |
| | IEd | | KKYRQY | | | KYRQ | | | | |
| 105 | H-2- | 40 | FVIDSVKVS | 5 | 123 | VKVSK | 0.753 | 0.337903 | 0.42 | <=SB |
| | IEk | | KKYRQY | | | KYRQ | | | | |
| 117 | H-2- | 41 | RQYLYNFM | 3 | 124 | LYNFM | 0.5 | 0.049673 | 4.31 | <=WB |
| | IEd | | ANLKNDT | | | ANLK | | | | |
| 141 | H-2- | 42 | FEGRVIVSTR | 4 | 125 | VIVSTR | 0.7 | 0.057075 | 3.6 | <=WB |
| | IEd | | HENNM | | | HEN | | | | |
| 142 | H-2- | 43 | EGRVIVSTR | 3 | 125 | VIVSTR | 0.727 | 0.359726 | 0.31 | <=SB |
| | IEk | | HENNMD | | | HEN | | | | |
| 154 | H-2- | 44 | NMDFGHSEA | 3 | 126 | FGHSEA | 0.953 | 0.455241 | 0.84 | <=SB |
| | IAu | | NTNYFK | | | NTN | | | | |
| 154 | H-2- | 44 | NMDFGHSEA | 3 | 126 | FGHSEA | 1 | 0.698596 | 0.48 | <=SB |
| | IAb | | NTNYFK | | | NTN | | | | |
| 163 | H-2- | 45 | NTNYFKKAV | 3 | 127 | YFKKA | 0.533 | 0.132776 | 0.83 | <=SB |
| | IEd | | EDYRQN | | | VEDY | | | | |
| 164 | H-2- | 46 | TNYFKKAVE | 3 | 128 | FKKAVE | 0.673 | 0.11127 | 1.2 | <=WB |
| | IEd | | DYRQNQ | | | DYR | | | | |
| 164 | H-2- | 46 | TNYFKKAVE | 3 | 128 | FKKAVE | 0.747 | 0.170496 | 4.82 | <=WB |
| | IEk | | DYRQNQ | | | DYR | | | | |
| 186 | H-2- | 47 | SNLSEGISAE | 3 | 129 | SEGISAE | 0.993 | 0.890552 | 0.04 | <=SB |
| | IAd | | VAIRS | | | VA | | | | |
| 186 | H-2- | 47 | SNLSEGISAE | 3 | 129 | SEGISAE | 0.667 | 0.308973 | 3.13 | <=WB |
| | IAu | | VAIRS | | | VA | | | | |
| 187 | H-2- | 48 | NLSEGISAEV | 5 | 130 | ISAEVAI | 0.633 | 0.36277 | 1.91 | <=WB |
| | IAu | | AIRSK | | | RS | | | | |
| 188 | H-2- | 49 | LSEGISAEVA | 4 | 130 | ISAEVAI | 0.953 | 0.31671 | 0.57 | <=SB |
| | IEk | | IRSKQ | | | RS | | | | |
| 189 | H-2- | 50 | SEGISAEVAI | 3 | 130 | ISAEVAI | 0.96 | 0.073634 | 2.49 | <=WB |
| | IEd | | RSKQS | | | RS | | | | |
| 189 | H-2- | 50 | SEGISAEVAI | 3 | 130 | ISAEVAI | 0.72 | 0.625238 | 0.88 | <=SB |
| | IAd | | RSKQS | | | RS | | | | |
| 189 | H-2- | 50 | SEGISAEVAI | 3 | 130 | ISAEVAI | 0.973 | 0.212758 | 3.85 | <=WB |
| | IAb | | RSKQS | | | RS | | | | |
| 192 | H-2- | 51 | ISAEVAIRSK | 4 | 131 | VAIRSK | 0.373 | 0.169527 | 4.88 | <=WB |
| | IEk | | QSEKK | | | QSE | | | | |
| 192 | H-2- | 51 | ISAEVAIRSK | 3 | 132 | EVAIRS | 1 | 0.834377 | 0.12 | <=SB |
| | IAd | | QSEKK | | | KOS | | | | |
| 193 | H-2- | 52 | SAEVAIRSK | 5 | 133 | IRSKQS | 0.633 | 0.238958 | 1.82 | <=WB |
| | IEk | | QSEKKA | | | EKK | | | | |
| 194 | H-2- | 53 | AEVAIRSKQ | 4 | 133 | IRSKQS | 0.967 | 0.261887 | 0.13 | <=SB |
| | IEd | | SEKKAF | | | EKK | | | | |
| 203 | H-2- | 54 | SEKKAFAIIV | 3 | 134 | KAFAIIV | 0.453 | 0.296421 | 3.48 | <=WB |
| | IAu | | PVYSP | | | PV | | | | |
| 204 | H-2- | 55 | EKKAFAIIVP | 4 | 135 | FAIIVPV | 0.953 | 0.062614 | 3.17 | <=WB |
| | IEd | | VYSPE | | | YS | | | | |
| 204 | H-2- | 55 | EKKAFAIIVP | 4 | 135 | FAIIVPV | 0.853 | 0.180104 | 4.21 | <=WB |
| | IEk | | VYSPE | | | YS | | | | |
| 204 | H-2- | 55 | EKKAFAIIVP | 4 | 135 | FAIIVPV | 0.647 | 0.328933 | 2.59 | <=WB |
| | IAu | | VYSPE | | | YS | | | | |
| 205 | H-2- | 56 | KKAFAIIVPV | 3 | 135 | FAIIVPV | 0.947 | 0.325219 | 2.33 | <=WB |
| | IAb | | YSPED | | | YS | | | | |
| 205 | H-2- | 56 | KKAFAIIVPV | 3 | 135 | FAIIVPV | 0.833 | 0.45916 | 2.41 | <=WB |
| | IAd | | YSPED | | | YS | | | | |
| 249 | H-2- | 57 | RGNFIYVDP | 3 | 136 | FIYVDP | 0.533 | 0.16003 | 0.53 | <=SB |
| | IEd | | NNIAVN | | | NNI | | | | |
| 249 | H-2- | 57 | RGNFIYVDP | 3 | 136 | FIYVDP | 0.747 | 0.578986 | 0.85 | <=SB |
| | IAb | | NNIAVN | | | NNI | | | | |
| 249 | H-2- | 57 | RGNFIYVDP | 3 | 136 | FIYVDP | 0.827 | 0.43732 | 1.01 | <=WB |
| | IAu | | NNIAVN | | | NNI | | | | |
| 250 | H-2- | 58 | GNFIYVDPN | 3 | 137 | IYVDPN | 0.72 | 0.138948 | 0.75 | <=SB |
| | IEd | | NIAVNP | | | NIA | | | | |
| 250 | H-2- | 58 | GNFIYVDPN | 3 | 137 | IYVDPN | 0.827 | 0.293 | 0.83 | <=SB |
| | IEk | | NIAVNP | | | NIA | | | | |
| 250 | H-2- | 58 | GNFIYVDPN | 4 | 138 | YVDPN | 0.56 | 0.526163 | 1.07 | <=WB |
| | IAb | | NIAVNP | | | NIAV | | | | |
| 275 | H-2- | 59 | SSKFLNVLK | 4 | 139 | LNVLKD | 0.607 | 0.177686 | 4.35 | <=WB |
| | IEk | | DVFSKP | | | VFS | | | | |
| 299 | H-2- | 60 | VSVYTIDRIL | 3 | 140 | YTIDRIL | 0.947 | 0.046298 | 4.7 | <=WB |
| | IEd | | LSEMG | | | LS | | | | |
| 314 | H-2- | 61 | EDCYYAMLP | 3 | 141 | YYAML | 0.98 | 0.186417 | 4.42 | <=WB |
| | IAb | | ISSKLG | | | PISS | | | | |
| 329 | H-2- | 62 | EKSGVLIARL | 4 | 142 | VLIARL | 0.66 | 0.167903 | 4.99 | <=WB |
| | IEk | | PYKDI | | | PYK | | | | |
| 330 | H-2- | 63 | KSGVLIARLP | 3 | 142 | VLIARL | 0.673 | 0.05067 | 4.2 | <=WB |
| | IEd | | YKDIY | | | PYK | | | | |
| 340 | H-2- | 64 | YKDIYGVISS | 3 | 143 | IYGVISS | 0.78 | 0.169606 | 4.88 | <=WB |
| | IEk | | LRFQY | | | LR | | | | |
| 376 | H-2- | 65 | IISFRLNAIRV | 3 | 144 | FRLNAI | 0.873 | 0.089265 | 1.81 | <=WB |
| | IEd | | LVQD | | | RVL | | | | |
| 381 | H-2- | 66 | LNAIRVLVQ | 3 | 145 | IR VL VQ | 0.727 | 0.183943 | 3.98 | <=WB |
| | IEk | | DMVKGN | | | DMV | | | | |
| 414 | H-2- | 67 | MLSLQVVK | 5 | 146 | VVKMK | 0.833 | 0.283069 | 0.97 | <=SB |
| | IEk | | MKKAISV | | | KAIS | | | | |
| 415 | H-2- | 68 | LSLQVVKMK | 4 | 146 | VVKMK | 0.653 | 0.047115 | 4.6 | <=WB |
| | IEd | | KAISVA | | | KAIS | | | | |
| 419 | H-2- | 69 | VVKMKKAIS | 4 | 147 | KKAISV | 0.78 | 0.354904 | 4.14 | <=WB |
| | IAd | | VAISSV | | | AIS | | | | |
| 427 | H-2- | 70 | SVAISSVLRN | 3 | 148 | ISSVLR | 0.907 | 0.194071 | 3.47 | <=WB |
| | IEk | | ISYVN | | | NIS | | | | |
| 434 | H-2- | 71 | LRNISYVNK | 3 | 149 | ISYVNK | 0.473 | 0.052784 | 3.98 | <=WB |
| | IEd | | ASLEVA | | | ASL | | | | |
| 434 | H-2- | 71 | LRNISYVNK | 3 | 149 | ISYVNK | 0.867 | 0.228041 | 2.15 | <=WB |
| | IEK | | ASLEVA | | | ASL | | | | |
| 435 | H-2- | 72 | RNISYVNKA | 4 | 150 | YVNKA | 0.633 | 0.07061 | 2.67 | <=WB |
| | IEd | | SLEVAS | | | SLEV | | | | |
| 439 | H-2- | 73 | YVNKASLEV | 3 | 151 | KASLEV | 0.733 | 0.364541 | 3.93 | <=WB |
| | IAd | | ASSSQN | | | ASS | | | | |
| 450 | H-2- | 74 | SSQNLSSSAL | 4 | 152 | LSSSAL | 0.74 | 0.370535 | 1.79 | <=WB |
| | IAu | | QQASA | | | QQA | | | | |
| 456 | H-2- | 75 | SSALQQASA | 3 | 153 | LQQASA | 0.78 | 0.302702 | 3.3 | <=WB |
| | IAu | | LEEMSA | | | LEE | | | | |
| 464 | H-2- | 76 | ALEEMSANV | 3 | 154 | EMSAN | 0.847 | 0.406234 | 3.18 | <=WB |
| | IAd | | EQIASG | | | VEQI | | | | |
| 471 | H-2- | 77 | NVEQIASGV | 3 | 155 | QIASGV | 0.787 | 0.508035 | 0.49 | <=SB |
| | IAu | | NMSANN | | | NMS | | | | |
| 471 | H-2- | 77 | NVEQIASGV | 3 | 155 | QIASGV | 0.853 | 0.195168 | 4.22 | <=WB |
| | IAb | | NMSANN | | | NMS | | | | |
| 471 | H-2- | 77 | NVEQIASGV | 3 | 155 | QIASGV | 0.853 | 0.461988 | 2.37 | <=WB |
| | IAd | | NMSANN | | | NMS | | | | |
| 484 | H-2- | 78 | NNSYETEQI | 3 | 156 | YETEQI | 0.987 | 0.105485 | 1.32 | <=WB |
| | IEd | | ALKTNE | | | ALK | | | | |
| 485 | H-2- | 79 | NSYETEQIAL | 3 | 157 | ETEQIA | 0.573 | 0.526016 | 1.62 | <=WB |
| | IAd | | KTNEN | | | LKT | | | | |
| 486 | H-2- | 80 | SYETEQIALK | 5 | 158 | QIALKT | 0.867 | 0.611826 | 0.95 | <=SB |
| | IAd | | TNENS | | | NEN | | | | |
| 488 | H-2- | 81 | ETEQIALKTN | 4 | 159 | IALKTN | 0.827 | 0.175319 | 4.49 | <=WB |
| | IEk | | ENSQI | | | ENS | | | | |
| 489 | H-2- | 82 | TEQIALKTNE | 3 | 159 | IALKTN | 0.733 | 0.059519 | 3.4 | <=WB |
| | IEd | | NSQIG | | | ENS | | | | |
| 490 | H-2- | 83 | EQIALKTNE | 4 | 160 | LKTNEN | 0.533 | 0.046829 | 4.63 | <=WB |
| | IEd | | NSQIGG | | | SQI | | | | |
| 505 | H-2- | 84 | RAVEESVIA | 3 | 161 | EESVIA | 0.987 | 0.590157 | 1.1 | <=WB |
| | IAd | | MQDIVE | | | MQD | | | | |
| 508 | H-2- | 85 | EESVIAMQDI | 4 | 162 | IAMQDI | 0.653 | 0.223288 | 2.3 | <=WB |
| | IEk | | VEKVS | | | VEK | | | | |
| 515 | H-2- | 86 | QDIVEKVSVI | 3 | 163 | VEKVSV | 0.56 | 0.454143 | 2.48 | <=WB |
| | IAd | | EEIAR | | | IEE | | | | |
| 516 | H-2- | 87 | DIVEKVSVIE | 3 | 164 | EKVSVI | 0.827 | 0.25741 | 4.94 | <=WB |
| | IAu | | EIARK | | | EEI | | | | |
| 516 | H-2- | 87 | DIVEKVSVIE | 3 | 164 | EKVSVI | 0.727 | 0.475709 | 2.19 | <=WB |
| | IAd | | EIARK | | | EEI | | | | |
| 518 | H-2- | 88 | VEK VSVIEEI | 3 | 165 | VSVIEEI | 0.693 | 0.226838 | 2.19 | <=WB |
| | IEk | | ARKTN | | | AR | | | | |
| 523 | H-2- | 89 | VIEEIARKTN | 4 | 166 | IARKTN | 0.86 | 0.175505 | 4.48 | <=WB |
| | IEk | | LLALN | | | LLA | | | | |
| 530 | H-2- | 90 | KTNLLALNA | 3 | 167 | LLALNA | 0.48 | 0.207111 | 2.89 | <=WB |
| | IEk | | AIEAAR | | | AIE | | | | |
| 530 | H-2- | 90 | KTNLLALNA | 3 | 167 | LLALNA | 0.613 | 0.347534 | 4.32 | <=WB |
| | IAd | | AIEAAR | | | AIE | | | | |
| 531 | H-2- | 91 | TNLLALNAA | 3 | 168 | LALNAA | 0.66 | 0.195525 | 3.41 | <=WB |
| | IEk | | IEAARA | | | IEA | | | | |
| 531 | H-2- | 91 | TNLLALNAA | 6 | 169 | NAAIEA | 0.72 | 0.572073 | 1.24 | <=WB |
| | IAd | | IEAARA | | | ARA | | | | |
| 535 | H-2- | 92 | ALNAAIEAA | 2 | 169 | NAAIEA | 0.613 | 0.451233 | 0.87 | <=SB |
| | IAu | | RAGDEG | | | ARA | | | | |
| 536 | H-2- | 93 | LNAAIEAAR | 4 | 170 | IEAARA | 0.787 | 0.456928 | 0.82 | <=SB |
| | IAu | | AGDEGK | | | GDE | | | | |
| 537 | H-2- | 94 | NAAIEAARA | 3 | 170 | IEAARA | 1 | 0.243691 | 3.3 | <=WB |
| | IAb | | GDEGKG | | | GDE | | | | |
| 537 | H-2- | 94 | NAAIEAARA | 3 | 170 | IEAARA | 0.893 | 0.471855 | 2.24 | <=WB |
| | IAd | | GDEGKG | | | GDE | | | | |
| 546 | H-2- | 95 | GDEGKGFAV | 3 | 171 | GKGFA | 0.387 | 0.25758 | 4.93 | <=WB |
| | IAu | | VASEIR | | | VVAS | | | | |
| 549 | H-2- | 96 | GKGFAVVAS | 3 | 172 | FAVVAS | 0.947 | 0.153508 | 0.59 | <=SB |
| | TEd | | EIRKLA | | | EIR | | | | |
| 549 | H-2- | 96 | GKGFAVVAS | 3 | 172 | FAVVAS | 0.713 | 0.209232 | 2.81 | <=WB |
| | IEk | | EIRKLA | | | EIR | | | | |
| 550 | H-2- | 97 | KGFAVVASE | 5 | 173 | VASEIR | 0.373 | 0.209472 | 2.8 | <=WB |
| | IEk | | IRKLAD | | | KLA | | | | |
| 550 | H-2- | 97 | KGFAVVASE | 4 | 174 | VVASEI | 0.953 | 0.385029 | 1.58 | <=WB |
| | IAu | | IRKLAD | | | RKL | | | | |
| 556 | H-2- | 98 | ASEIRKLADL | 3 | 175 | IRKLAD | 0.8 | 0.190744 | 3.64 | <=WB |
| | IEk | | SKISA | | | LSK | | | | |
| 558 | H-2- | 99 | EIRKLADLSK | 4 | 176 | LADLSK | 0.847 | 0.174329 | 4.56 | <=WB |
| | IEk | | ISALE | | | ISA | | | | |
| 573 | H-2- | 100 | IGEL VEDNS | 3 | 177 | LVEDNS | 0.647 | 0.169842 | 4.86 | <=WB |
| | IEk | | KVATEA | | | KVA | | | | |
| 601 | H-2- | 101 | TANLVKKIS | 3 | 178 | LVKKIS | 0.867 | 0.082074 | 2.1 | <=WB |
| | IEd | | EGSSKQ | | | EGS | | | | |
| 601 | H-2- | 101 | TANLVKKIS | 3 | 178 | LVKKIS | 0.627 | 0.247651 | 1.61 | <=WB |
| | IEk | | EGSSKQ | | | EGS | | | | |
| 602 | H-2- | 102 | ANL VKKISE | 3 | 179 | VKKISE | 0.52 | 0.197377 | 3.32 | <=WB |
| | IEk | | GSSKQS | | | GSS | | | | |
| 626 | H-2- | 103 | LDQVGEVVQ | 2 | 180 | QVGEV | 0.667 | 0.356556 | 4.11 | <=WB |
| | IAd | | SSASSS | | | VOSS | | | | |
| 627 | H-2- | 104 | DQVGEVVQS | 5 | 181 | VVOSSA | 0.447 | 0.345542 | 4.36 | <=WB |
| | IAd | | SASSSE | | | SSS | | | | |
| 628 | H-2- | 105 | QVGEVVQSS | 4 | 181 | VVQSSA | 0.893 | 0.554674 | 0.29 | <=SB |
| | IAu | | ASSSEQ | | | SSS | | | | |
| 628 | H-2- | 105 | QVGEVVQSS | 4 | 181 | VVOSSA | 0.98 | 0.408789 | 1.66 | <=WB |
| | IAb | | ASSSEQ | | | SSS | | | | |
| 647 | H-2- | 106 | SDKMLEKSK | 4 | 182 | LEKSKE | 0.56 | 0.058347 | 3.49 | <=WB |
| | IEd | | ELRKSV | | | LRK | | | | |
| 660 | H-2- | 107 | SVLFFKIKDS | 4 | 183 | FKIKDS | 0.633 | 0.140332 | 0.73 | <=SB |
| | IEd | | KIENP | | | KIE | | | | |
| 687 | H-2- | 108 | PENSFKDEN | 4 | 184 | FKDENQ | 0.96 | 0.058712 | 3.46 | <=WB |
| | IEd | | QNLKSN | | | NLK | | | | |
| 697 | H-2- | 109 | NLKSNGISTS | 3 | 185 | SNGISTS | 0.907 | 0.431095 | 2.81 | <=WB |
| | IAd | | NASGH | | | NA | | | | |
| 699 | H-2- | 110 | KSNGISTSNA | 4 | 186 | ISTSNAS | 0.66 | 0.30527 | 3.23 | <=WB |
| | IAu | | SGHNN | | | GH | | | | |
| 720 | H-2- | 111 | SESSVRTINK | 4 | 187 | VRTINK | 0.92 | 0.314656 | 0.59 | <=SB |
| | IEk | | RVDPK | | | RVD | | | | |
| 721 | H-2- | 112 | ESSVRTINKR | 3 | 187 | VRTINK | 0.92 | 0.121399 | 1 | <=SB |
| | IEd | | VDPKK | | | RVD | | | | |
| 724 | H-2- | 113 | VRTINKRVD | 3 | 188 | INKRVD | 0.78 | 0.055738 | 3.72 | <=WB |
| | IEd | | PKKAID | | | PKK | | | | |
| 734 | H-2- | 114 | KKAIDIADK | 3 | 189 | IDIADK | 0.973 | 0.328616 | 0.48 | <=SB |
| | IEk | | DLNFDD | | | DLN | | | | |

Table 2 below shows predicted human MHCII binding of peptides derived from BB0680 determined using NetMHCIIpan (V 4.0). The prediction outcome for each molecule includes the following columns in the table: Pos Residue number (starting from 0); MHC MHC molecule name; Peptide Amino acid sequence; Of Starting position offset of the optimal binding core (starting from 0); Core Binding core register; Core_Rel Reliability of the binding core, expressed as the fraction of networks in the ensemble selecting the optimal core; Score_EL Eluted ligand prediction score; % Rank_EL Percentile rank of eluted ligand prediction score: BindLevel (SB: strong binder, WE: weak binder). The peptide will be identified as a strong binder if the % Rank is below the 1% threshold for the strong binders. The peptide will be identified as a weak binder if the % Rank is above the 1% threshold of the strong binders but below the 5% threshold for the weak binders.

TABLE 2
Predicted human MHC II binding of peptides derived from BB0680 determined
using NetMHCIIpan (V.4.0)

| | | SEQ ID | | | SEQ ID | | Core_ | Score_ | % Rank_ | |
|---|---|---|---|---|---|---|---|---|---|---|
| Pos | MHC | NO: | Peptide | Of | NO: | Core | Rel | EL | EL | BindLevel |
| 36 | DRB5_ | 190 | LEDYYKQLTRA | 4 | 370 | YKQLTRA | 0.587 | 0.531712 | 1.65 | <=WB |
| | 0101 | | QVRR | | | QV | | | | |
| 36 | DRB1_ | 190 | LEDYYKQLTRA | 3 | 371 | YYKQLTR | 0.947 | 0.678389 | 0.93 | <=SB |
| | 0401 | | QVRR | | | AC | | | | |
| 36 | DRB1_ | 190 | LEDYYKQLTRA | 3 | 371 | YYKQLTR | 0.947 | 0.654002 | 1.27 | <=WB |
| | 0408 | | QVRR | | | AQ | | | | |
| 36 | DRB1_ | 190 | LEDYYKQLTRA | 4 | 370 | YKQLTRA | 0.947 | 0.782222 | 0.08 | <=SB |
| | 0803 | | QVRR | | | QV | | | | |
| 36 | DRB1_ | 190 | LEDYYKQLTRA | 3 | 371 | YYKQLTR | 0.907 | 0.361403 | 2.96 | <=WB |
| | 0402 | | QVRR | | | AQ | | | | |
| 36 | DRB1_ | 190 | LEDYYKQLTRA | 3 | 371 | YYKQLTR | 0.867 | 0.472593 | 3.31 | <=WB |
| | 0403 | | QVRR | | | AQ | | | | |
| 36 | DRB1_ | 190 | LEDYYKQLTRA | 3 | 371 | YYKQLTR | 0.773 | 0.349408 | 3.25 | <=WB |
| | 0405 | | QVRR | | | AQ | | | | |
| 36 | DRB1_ | 190 | LEDYYKQLTRA | 3 | 371 | YYKQLTR | 0.72 | 0.589437 | 2.02 | <=WB |
| | 1001 | | QVRR | | | AQ | | | | |
| 36 | DRB4_ | 190 | LEDYYKQLTRA | 3 | 371 | YYKQLTR | 0.66 | 0.124226 | 4.35 | <=WB |
| | 0101 | | QVRR | | | AQ | | | | |
| 36 | DRB4_ | 190 | LEDYYKQLTRA | 3 | 371 | YYKQLTR | 0.66 | 0.124226 | 4.35 | <=WB |
| | 0103 | | QVRR | | | AQ | | | | |
| 37 | DRB1_ | 191 | EDYYKQLTRA | 3 | 370 | YKQLTRA | 0.987 | 0.649863 | 0.21 | <=SB |
| | 1302 | | QVRRA | | | QV | | | | |
| 37 | DRB1_ | 191 | EDYYKQLTRA | 3 | 370 | YKQLTRA | 0.993 | 0.869442 | 0.29 | <=SB |
| | 1303 | | QVRRA | | | QV | | | | |
| 37 | DRB1_ | 191 | EDYYKQLTRA | 3 | 370 | YKQLTRA | 0.993 | 0.658188 | 0.01 | <=SB |
| | 1402 | | QVRRA | | | QV | | | | |
| 37 | DRB3_ | 191 | EDYYKQLTRA | 3 | 370 | YKQLTRA | 0.76 | 0.160669 | 2.93 | <=WB |
| | 0202 | | QVRRA | | | QV | | | | |
| 37 | DRB1_ | 191 | EDYYKQLTRA | 3 | 370 | YKQLTRA | 0.973 | 0.930734 | 0.09 | <=SB |
| | 0801 | | QVRRA | | | QV | | | | |
| 37 | DRB1_ | 191 | EDYYKQLTRA | 3 | 370 | YKQLTRA | 1 | 0.983828 | 0.02 | <=SB |
| | 1101 | | QVRRA | | | QV | | | | |
| 37 | DRB1_ | 191 | EDYYKQLTRA | 3 | 370 | YKQLTRA | 1 | 0.82429 | 0.76 | <=SB |
| | 1104 | | QVRRA | | | QV | | | | |
| 37 | DRB1_ | 191 | EDYYKQLTRA | 3 | 370 | YKQLTRA | 1 | 0.665334 | 0.62 | <=SB |
| | 1401 | | QVRRA | | | QV | | | | |
| 37 | DRB1_ | 191 | EDYYKQLTRA | 3 | 370 | YKQLTRA | 1 | 0.665334 | 0.62 | <=SB |
| | 1454 | | QVRRA | | | QV | | | | |
| 37 | DRB1_ | 191 | EDYYKQLTRA | 3 | 370 | YKQLTRA | 0.787 | 0.498132 | 1.59 | <=WB |
| | 0103 | | QVRRA | | | QV | | | | |
| 37 | DRB1_ | 191 | EDYYKQLTRA | 3 | 370 | YKQLTRA | 0.96 | 0.124013 | 3.97 | <=WB |
| | 0305 | | QVRRA | | | QV | | | | |
| 37 | DRB1_ | 191 | EDYYKQLTRA | 3 | 370 | YKQLTRA | 0.487 | 0.517136 | 2.79 | <=WB |
| | 1001 | | QVRRA | | | QV | | | | |
| 37 | DRB1_ | 191 | EDYYKQLTRA | 3 | 370 | YKQLTRA | 0.907 | 0.3578 | 3.01 | <=WB |
| | 1201 | | QVRRA | | | QV | | | | |
| 37 | DRB1_ | 191 | EDYYKQLTRA | 3 | 370 | YKQLTRA | 0.967 | 0.530307 | 3.2 | <=WB |
| | 1301 | | QVRRA | | | QV | | | | |
| 37 | DRB1_ | 191 | EDYYKQLTRA | 3 | 370 | YKQLTRA | 0.893 | 0.393541 | 3.82 | <=WB |
| | 1601 | | QVRRA | | | QV | | | | |
| 40 | DRB1_ | 192 | YKQLTRAQVR | 3 | 372 | LTRAQVR | 0.987 | 0.369447 | 2.86 | <=WB |
| | 0402 | | RAAFS | | | RA | | | | |
| 40 | DRB4_ | 192 | YKQLTRAQVR | 3 | 372 | LTRAQVR | 0.9 | 0.151118 | 3.36 | <=WB |
| | 0101 | | RAAFS | | | RA | | | | |
| 40 | DRB4_ | 192 | YKQLTRAQVR | 3 | 372 | LTRAQVR | 0.9 | 0.151118 | 3.36 | <=WB |
| | 0103 | | RAAFS | | | RA | | | | |
| 58 | DRB1_ | 193 | FLDTLHVIINGA | 4 | 373 | LHVIINGA | 0.947 | 0.395462 | 3.09 | <=WB |
| | 0401 | | ASN | | | A | | | | |
| 58 | DRB1_ | 193 | FLDTLHVIINGA | 4 | 373 | LHVIINGA | 0.987 | 0.634941 | 1.39 | <=WB |
| | 0408 | | ASN | | | A | | | | |
| 58 | DRB1_ | 193 | FLDTLHVIINGA | 4 | 373 | LHVIINGA | 1 | 0.561028 | 1.18 | <=WB |
| | 0402 | | ASN | | | A | | | | |
| 58 | DRB1_ | 193 | FLDTLHVIINGA | 4 | 373 | LHVIINGA | 0.973 | 0.567863 | 2.15 | <=WB |
| | 0403 | | ASN | | | A | | | | |
| 58 | DRB1_ | 193 | FLDTLHVIINGA | 4 | 373 | LHVIINGA | 0.98 | 0.294147 | 4.07 | <=WB |
| | 0405 | | ASN | | | A | | | | |
| 58 | DRB1_ | 193 | FLDTLHVIINGA | 4 | 373 | LHVIINGA | 0.993 | 0.414379 | 4.6 | <=WB |
| | 1104 | | ASN | | | A | | | | |
| 58 | DRB1_ | 193 | FLDTLHVIINGA | 4 | 373 | LHVIINGA | 0.993 | 0.330138 | 2.97 | <=WB |
| | 1501 | | ASN | | | A | | | | |
| 58 | DRB1_ | 193 | FLDTLHVIINGA | 4 | 373 | LHVIINGA | 0.987 | 0.217371 | 3.71 | <=WB |
| | 1503 | | ASN | | | A | | | | |
| 58 | DRB1_ | 193 | FLDTLHVIINGA | 4 | 373 | LHVIINGA | 0.993 | 0.398665 | 3.75 | <=WB |
| | 1601 | | ASN | | | A | | | | |
| 60 | DRB1_ | 194 | DTLHVIINGAA | 4 | 374 | VIINGAAS | 0.847 | 0.383412 | 2.59 | <=WB |
| | 1302 | | SNLA | | | N | | | | |
| 60 | DRB1_ | 194 | DTLHVIINGAA | 4 | 374 | VIINGAAS | 0.813 | 0.202218 | 3.01 | <=WB |
| | 1402 | | SNLA | | | N | | | | |
| 60 | DRB3_ | 194 | DTLHVIINGAA | 4 | 374 | VIINGAAS | 0.993 | 0.655427 | 0.33 | <=SB |
| | 0202 | | SNLA | | | N | | | | |
| 60 | DRB1_ | 194 | DTLHVIINGAA | 2 | 373 | LHVIINGA | 0.98 | 0.685259 | 1.26 | <=WB |
| | 0404 | | SNLA | | | A | | | | |
| 61 | DRB1_ | 195 | TLHVIINGAAS | 5 | 375 | INGAASN | 0.86 | 0.452553 | 3.21 | <=WB |
| | 0404 | | NLAL | | | LA | | | | |
| 62 | DRB1_ | 196 | LHVIINGAASN | 4 | 375 | INGAASN | 0.693 | 0.514398 | 2.07 | <=WB |
| | 0901 | | LALE | | | LA | | | | |
| 63 | DRB1_ | 197 | HVIINGAASNL | 3 | 375 | INGAASN | 1 | 0.522184 | 1.93 | <=WB |
| | 0401 | | ALET | | | LA | | | | |
| 63 | DRB1_ | 197 | HVIINGAASNL | 3 | 375 | INGAASN | 1 | 0.534776 | 2.22 | <=WB |
| | 0408 | | ALET | | | LA | | | | |
| 63 | DRB1_ | 197 | HVIINGAASNL | 3 | 375 | INGAASN | 0.84 | 0.496205 | 3.11 | <=WB |
| | 0102 | | ALET | | | LA | | | | |
| 63 | DRB1_ | 197 | HVIINGAASNL | 3 | 375 | INGAASN | 0.993 | 0.404634 | 2.43 | <=WB |
| | 0402 | | ALET | | | LA | | | | |
| 63 | DRB1_ | 197 | HVIINGAASNL | 3 | 375 | INGAASN | 0.987 | 0.435795 | 3.87 | <=WB |
| | 0403 | | ALET | | | LA | | | | |
| 63 | DRB1_ | 197 | HVIINGAASNL | 3 | 375 | INGAASN | 0.967 | 0.214451 | 4.68 | <=WB |
| | 1501 | | ALET | | | LA | | | | |
| 72 | DRB1_ | 198 | NLALETISEFA | 3 | 376 | LETISEFA | 0.993 | 0.27166 | 4.96 | <=WB |
| | 1201 | | MSEN | | | M | | | | |
| 78 | DRB5_ | 199 | ISEFAMSENRG | 3 | 377 | FAMSENR | 1 | 0.79721 | 0.36 | <=SB |
| | 0101 | | KDFS | | | GK | | | | |
| 92 | DRB1_ | 200 | SESELIDLRKNP | 4 | 378 | LIDLRKN | 0.987 | 0.513876 | 3.28 | <=WB |
| | 1104 | | KFV | | | PK | | | | |
| 93 | DRB1_ | 201 | ESELIDLRKNPK | 4 | 379 | IDLRKNP | 0.46 | 0.31168 | 4.47 | <=WB |
| | 1302 | | FVI | | | KF | | | | |
| 93 | DRB1_ | 201 | ESELIDLRKNPK | 3 | 378 | LIDLRKN | 0.593 | 0.594023 | 2.4 | <=WB |
| | 1301 | | FVI | | | PK | | | | |
| 94 | DRB1_ | 202 | SELIDLRKNPKF | 5 | 380 | LRKNPKF | 0.673 | 0.511795 | 0.92 | <=SB |
| | 1302 | | VID | | | VI | | | | |
| 94 | DRB1_ | 202 | SELIDLRKNPKF | 3 | 379 | IDLRKNP | 0.473 | 0.636043 | 1.95 | <=WB |
| | 1301 | | VID | | | KF | | | | |
| 94 | DRB5_ | 202 | SELIDLRKNPKF | 3 | 379 | IDLRKNP | 0.833 | 0.276376 | 3.61 | <=WB |
| | 0202 | | VID | | | KF | | | | |
| 95 | DRB1_ | 203 | ELIDLRKNPKF | 4 | 380 | LRKNPKF | 0.793 | 0.60364 | 2.3 | <=WB |
| | 1301 | | VIDS | | | VI | | | | |
| 95 | DRB1_ | 203 | ELIDLRKNPKF | 4 | 380 | LRKNPKF | 0.987 | 0.577142 | 1.09 | <=WB |
| | 1401 | | VIDS | | | VI | | | | |
| 95 | DRB1_ | 203 | ELIDLRKNPKF | 4 | 380 | LRKNPKF | 0.987 | 0.577142 | 1.09 | <=WB |
| | 1454 | | VIDS | | | VI | | | | |
| 96 | DRB1_ | 204 | LIDLRKNPKFVI | 3 | 380 | LRKNPKF | 1 | 0.248647 | 4.54 | <=WB |
| | 0301 | | DSV | | | VI | | | | |
| 96 | DRB1_ | 204 | LIDLRKNPKFVI | 3 | 380 | LRKNPKF | 0.973 | 0.106236 | 4.57 | <=WB |
| | 0305 | | DSV | | | VI | | | | |
| 101 | DRB1_ | 205 | KNPKFVIDSVK | 4 | 381 | FVIDSVK | 1 | 0.969728 | 0.02 | <=SB |
| | 0401 | | VSKK | | | VS | | | | |
| 101 | DRB1_ | 205 | KNPKFVIDSVK | 4 | 381 | FVIDSVK | 0.993 | 0.83448 | 0.39 | <=SB |
| | 1303 | | VSKK | | | VS | | | | |
| 101 | DRB1_ | 205 | KNPKFVIDSVK | 4 | 381 | FVIDSVK | 0.987 | 0.719195 | 0 | <=SB |
| | 1402 | | VSKK | | | VS | | | | |
| 101 | DRB1_ | 205 | KNPKFVIDSVK | 4 | 381 | FVIDSVK | 0.967 | 0.662691 | 0.3 | <=SB |
| | 0803 | | VSKK | | | VS | | | | |
| 101 | DRB3_ | 205 | KNPKFVIDSVK | 4 | 381 | FVIDSVK | 1 | 0.854598 | 0.13 | <=SB |
| | 0101 | | VSKK | | | VS | | | | |
| 101 | DRB1_ | 205 | KNPKFVIDSVK | 4 | 381 | FVIDSVK | 0.573 | 0.263594 | 4.49 | <=WB |
| | 0701 | | VSKK | | | VS | | | | |
| 101 | DRB1_ | 205 | KNPKFVIDSVK | 4 | 381 | FVIDSVK | 0.793 | 0.357228 | 4.84 | <=WB |
| | 0901 | | VSKK | | | VS | | | | |
| 102 | DRB1_ | 206 | NPKFVIDSVKV | 3 | 381 | FVIDSVK | 1 | 0.92195 | 0.1 | <=SB |
| | 0408 | | SKKY | | | VS | | | | |
| 102 | DRB1_ | 206 | NPKFVIDSVKV | 3 | 381 | FVIDSVK | 0.953 | 0.758144 | 0.04 | <=SB |
| | 1302 | | SKKY | | | VS | | | | |
| 102 | DRB3_ | 206 | NPKFVIDSVKV | 3 | 381 | FVIDSVK | 1 | 0.807297 | 0.14 | <=SB |
| | 0202 | | SKKY | | | VS | | | | |
| 102 | DRB5_ | 206 | NPKFVIDSVKV | 3 | 381 | FVIDSVK | 0.887 | 0.648682 | 0.95 | <=SB |
| | 0101 | | SKKY | | | VS | | | | |
| 102 | DRB1_ | 206 | NPKFVIDSVKV | 3 | 381 | FVIDSVK | 0.947 | 0.762544 | 0.73 | <=SB |
| | 0301 | | SKKY | | | VS | | | | |
| 102 | DRB1_ | 206 | NPKFVIDSVKV | 3 | 381 | FVIDSVK | 1 | 0.80192 | 0.04 | <=SB |
| | 0305 | | SKKY | | | VS | | | | |
| 102 | DRB1_ | 206 | NPKFVIDSVKV | 3 | 381 | FVIDSVK | 0.993 | 0.767226 | 0.65 | <=SB |
| | 0403 | | SKKY | | | VS | | | | |
| 102 | DRB1_ | 206 | NPKFVIDSVKV | 3 | 381 | FVIDSVK | 1 | 0.801173 | 0.5 | <=SB |
| | 1001 | | SKKY | | | VS | | | | |
| 102 | DRB1_ | 206 | NPKFVIDSVKV | 3 | 381 | FVIDSVK | 1 | 0.826692 | 0.8 | <=SB |
| | 1101 | | SKKY | | | VS | | | | |
| 102 | DRB4_ | 206 | NPKFVIDSVKV | 3 | 381 | FVIDSVK | 0.96 | 0.361946 | 0.78 | <=SB |
| | 0101 | | SKKY | | | VS | | | | |
| 102 | DRB4_ | 206 | NPKFVIDSVKV | 3 | 381 | FVIDSVK | 0.96 | 0.361946 | 0.78 | <=SB |
| | 0103 | | SKKY | | | VS | | | | |
| 102 | DRB1_ | 206 | NPKFVIDSVKV | 3 | 381 | FVIDSVK | 1 | 0.481133 | 2.69 | <=WB |
| | 0101 | | SKKY | | | VS | | | | |
| 102 | DRB1_ | 206 | NPKFVIDSVKV | 3 | 381 | FVIDSVK | 0.947 | 0.380733 | 2.67 | <=WB |
| | 0103 | | SKKY | | | VS | | | | |
| 102 | DRB1_ | 206 | NPKFVIDSVKV | 3 | 381 | FVIDSVK | 0.927 | 0.436034 | 2.1 | <=WB |
| | 0402 | | SKKY | | | VS | | | | |
| 102 | DRB1_ | 206 | NPKFVIDSVKV | 3 | 381 | FVIDSVK | 0.987 | 0.603914 | 1.83 | <=WB |
| | 0404 | | SKKY | | | VS | | | | |
| 102 | DRB1_ | 206 | NPKFVIDSVKV | 3 | 381 | FVIDSVK | 1 | 0.602687 | 1.26 | <=WB |
| | 0405 | | SKKY | | | VS | | | | |
| 102 | DRB1_ | 206 | NPKFVIDSVKV | 3 | 381 | FVIDSVK | 0.967 | 0.703248 | 1.27 | <=WB |
| | 0801 | | SKKY | | | VS | | | | |
| 102 | DRB1_ | 206 | NPKFVIDSVKV | 3 | 381 | FVIDSVK | 0.56 | 0.553793 | 2.88 | <=WB |
| | 1301 | | SKKY | | | VS | | | | |
| 102 | DRB1_ | 206 | NPKFVIDSVKV | 3 | 381 | FVIDSVK | 0.813 | 0.412151 | 2.77 | <=WB |
| | 1401 | | SKKY | | | VS | | | | |
| 102 | DRB1_ | 206 | NPKFVIDSVKV | 3 | 381 | FVIDSVK | 0.813 | 0.412151 | 2.77 | <=WB |
| | 1454 | | SKKY | | | VS | | | | |
| 102 | DRB1_ | 206 | NPKFVIDSVKV | 3 | 381 | FVIDSVK | 0.967 | 0.428318 | 3.36 | <=WB |
| | 1601 | | SKKY | | | VS | | | | |
| 102 | DRB5_ | 206 | NPKFVIDSVKV | 3 | 381 | FVIDSVK | 0.6 | 0.304836 | 3.11 | <=WB |
| | 0202 | | SKKY | | | VS | | | | |
| 103 | DRB5_ | 207 | PKFVIDSVKVS | 4 | 382 | IDSVKVS | 0.52 | 0.588907 | 1.27 | <=WB |
| | 0101 | | KKYR | | | KK | | | | |
| 103 | DRB1_ | 207 | PKFVIDSVKVS | 2 | 381 | FVIDSVK | 0.453 | 0.44022 | 4.22 | <=WB |
| | 1104 | | KKYR | | | VS | | | | |
| 103 | DRB5_ | 207 | PKFVIDSVKVS | 4 | 382 | IDSVKVS | 0.733 | 0.337901 | 2.64 | <=WB |
| | 0202 | | KKYR | | | KK | | | | |
| 103 | DRB1_ | 207 | PKFVIDSVKVS | 3 | 383 | VIDSVKV | 0.713 | 0.562255 | 2.78 | <=WB |
| | 1301 | | KKYR | | | SK | | | | |
| 104 | DRB1_ | 208 | KFVIDSVKVSK | 6 | 384 | VKVSKKY | 0.753 | 0.524022 | 3.17 | <=WB |
| | 1104 | | KYRQ | | | RQ | | | | |
| 104 | DRB1_ | 208 | KFVIDSVKVSK | 6 | 384 | VKVSKKY | 0.633 | 0.575896 | 2.61 | <=WB |
| | 1301 | | KYRQ | | | RQ | | | | |
| 106 | DRB1_ | 209 | VIDSVKVSKKY | 4 | 384 | VKVSKKY | 1 | 0.368611 | 3.93 | <=WB |
| | 1303 | | RQYL | | | RQ | | | | |
| 106 | DRB1_ | 209 | VIDSVKVSKKY | 4 | 384 | VKVSKKY | 0.993 | 0.246477 | 4.58 | <=WB |
| | 0301 | | RQYL | | | RQ | | | | |
| 107 | DRB1_ | 210 | IDSVKVSKKYR | 3 | 384 | VKVSKKY | 0.947 | 0.438296 | 1.71 | <=WB |
| | 1302 | | QYLY | | | RQ | | | | |
| 107 | DRB1_ | 210 | IDSVKVSKKYR | 3 | 384 | VKVSKKY | 1 | 0.679547 | 1.65 | <=WB |
| | 1101 | | QYLY | | | RQ | | | | |
| 107 | DRB1_ | 210 | IDSVKVSKKYR | 3 | 384 | VKVSKKY | 0.98 | 0.543765 | 1.35 | <=WB |
| | 1401 | | QYLY | | | RQ | | | | |
| 107 | DRB1_ | 210 | IDSVKVSKKYR | 3 | 384 | VKVSKKY | 0.98 | 0.543765 | 1.35 | <=WB |
| | 1454 | | QYLY | | | RQ | | | | |
| 109 | DRB1_ | 211 | SVKVSKKYRQ | 3 | 385 | VSKKYRQ | 0.72 | 0.557391 | 2.84 | <=WB |
| | 1301 | | YLYNF | | | YL | | | | |
| 116 | DRB1_ | 212 | YRQYLYNFMA | 3 | 386 | YLYNFM | 0.6 | 0.161737 | 4.65 | <=WB |
| | 1402 | | NLKND | | | ANL | | | | |
| 116 | DRB1_ | 212 | YRQYLYNFMA | 3 | 386 | YLYNFM | 0.393 | 0.377521 | 2.62 | <=WB |
| | 0803 | | NLKND | | | ANL | | | | |
| 117 | DRB5_ | 213 | RQYLYNFMAN | 3 | 387 | LYNFMA | 0.68 | 0.513893 | 1.78 | <=WB |
| | 0101 | | LKNDT | | | NLK | | | | |
| 117 | DRB3_ | 213 | RQYLYNFMAN | 2 | 386 | YLYNFM | 0.673 | 0.306277 | 1.4 | <=WB |
| | 0202 | | LKNDT | | | ANL | | | | |
| 117 | DRB1_ | 213 | RQYLYNFMAN | 4 | 388 | YNFMAN | 1 | 0.699428 | 0.84 | <=SB |
| | 0401 | | LKNDT | | | LKN | | | | |
| 117 | DRB1_ | 213 | RQYLYNFMAN | 4 | 388 | YNFMAN | 1 | 0.843525 | 0.32 | <=SB |
| | 0408 | | LKNDT | | | LKN | | | | |
| 117 | DRB1_ | 213 | RQYLYNFMAN | 3 | 387 | LYNFMA | 0.6 | 0.751437 | 0.67 | <=SB |
| | 1601 | | LKNDT | | | NLK | | | | |
| 117 | DRB5_ | 213 | RQYLYNFMAN | 3 | 387 | LYNFMA | 0.967 | 0.681585 | 0.44 | <=SB |
| | 0202 | | LKNDT | | | NLK | | | | |
| 117 | DRB1_ | 213 | RQYLYNFMAN | 4 | 388 | YNFMAN | 0.993 | 0.664354 | 1 | <=SB |
| | 0405 | | LKNDT | | | LKN | | | | |
| 117 | DRB1_ | 213 | RQYLYNFMAN | 4 | 388 | YNFMAN | 0.987 | 0.861953 | 0.25 | <=SB |
| | 1001 | | LKNDT | | | LKN | | | | |
| 117 | DRB1_ | 213 | RQYLYNFMAN | 3 | 387 | LYNFMA | 0.893 | 0.280407 | 3.55 | <=WB |
| | 1501 | | LKNDT | | | NLK | | | | |
| 117 | DRB1_ | 213 | RQYLYNFMAN | 3 | 387 | LYNFMA | 0.94 | 0.288374 | 2.56 | <=WB |
| | 1503 | | LKNDT | | | NLK | | | | |
| 117 | DRB1_ | 213 | RQYLYNFMAN | 4 | 388 | YNFMAN | 0.967 | 0.399213 | 3.41 | <=WB |
| | 0101 | | LKNDT | | | LKN | | | | |
| 117 | DRB1_ | 213 | RQYLYNFMAN | 4 | 388 | YNFMAN | 0.62 | 0.29645 | 3.85 | <=WB |
| | 0103 | | LKNDT | | | LKN | | | | |
| 117 | DRB1_ | 213 | RQYLYNFMAN | 4 | 388 | YNFMAN | 0.62 | 0.307283 | 3.81 | <=WB |
| | 0402 | | LKNDT | | | LKN | | | | |
| 117 | DRB1_ | 213 | RQYLYNFMAN | 4 | 388 | YNFMAN | 0.96 | 0.669789 | 1.26 | <=WB |
| | 0403 | | LKNDT | | | LKN | | | | |
| 117 | DRB1_ | 213 | RQYLYNFMAN | 4 | 388 | YNFMAN | 0.92 | 0.458481 | 3.14 | <=WB |
| | 0404 | | LKNDT | | | LKN | | | | |
| 117 | DRB1_ | 213 | RQYLYNFMAN | 4 | 388 | YNFMAN | 0.907 | 0.474014 | 3.99 | <=WB |
| | 0801 | | LKNDT | | | LKN | | | | |
| 117 | DRB1_ | 213 | RQYLYNFMAN | 4 | 388 | YNFMAN | 0.66 | 0.390594 | 2.4 | <=WB |
| | 0803 | | LKNDT | | | LKN | | | | |
| 117 | DRB1_ | 213 | RQYLYNFMAN | 4 | 388 | YNFMAN | 0.967 | 0.433545 | 3.82 | <=WB |
| | 1101 | | LKNDT | | | LKN | | | | |
| 118 | DRB3_ | 214 | QYLYNFMANL | 3 | 388 | YNFMAN | 0.547 | 0.12527 | 3.82 | <=WB |
| | 0202 | | KNDTL | | | LKN | | | | |
| 118 | DRB5_ | 214 | QYLYNFMANL | 3 | 388 | YNFMAN | 0.54 | 0.313203 | 4.3 | <=WB |
| | 0101 | | KNDTL | | | LKN | | | | |
| 118 | DRB1_ | 214 | QYLYNFMANL | 3 | 388 | YNFMAN | 0.613 | 0.550951 | 1.99 | <=WB |
| | 1601 | | KNDTL | | | LKN | | | | |
| 135 | DRB1_ | 215 | EFAFFDFEGRVI | 3 | 389 | FFDFEGR | 0.987 | 0.342124 | 3.14 | <=WB |
| | 0103 | | VST | | | VI | | | | |
| 135 | DRB1_ | 215 | EFAFFDFEGRVI | 3 | 389 | FFDFEGR | 0.987 | 0.270115 | 4.38 | <=WB |
| | 0701 | | VST | | | VI | | | | |
| 135 | DRB1_ | 215 | EFAFFDFEGRVI | 3 | 389 | FFDFEGR | 0.98 | 0.434516 | 3.28 | <=WB |
| | 1601 | | VST | | | VI | | | | |
| 142 | DRB1_ | 216 | EGRVIVSTRHE | 3 | 390 | VIVSTRH | 0.8 | 0.492495 | 1.11 | <=WB |
| | 1302 | | NNMD | | | EN | | | | |
| 142 | DRB1_ | 216 | EGRVIVSTRHE | 3 | 390 | VIVSTRH | 0.813 | 0.650837 | 1.24 | <=WB |
| | 1303 | | NNMD | | | EN | | | | |
| 142 | DRB1_ | 216 | EGRVIVSTRHE | 3 | 390 | VIVSTRH | 0.7 | 0.247067 | 1.85 | <=WB |
| | 1402 | | NNMD | | | EN | | | | |
| 142 | DRB1_ | 216 | EGRVIVSTRHE | 3 | 390 | VIVSTRH | 0.96 | 0.855553 | 0.59 | <=SB |
| | 1104 | | NNMD | | | EN | | | | |
| 142 | DRB1_ | 216 | EGRVIVSTRHE | 3 | 390 | VIVSTRH | 0.967 | 0.796378 | 0.64 | <=SB |
| | 1301 | | NNMD | | | EN | | | | |
| 142 | DRB1_ | 216 | EGRVIVSTRHE | 3 | 390 | VIVSTRH | 0.767 | 0.687451 | 0.54 | <=SB |
| | 1401 | | NNMD | | | EN | | | | |
| 142 | DRB1_ | 216 | EGRVIVSTRHE | 3 | 390 | VIVSTRH | 0.767 | 0.687451 | 0.54 | <=SB |
| | 1454 | | NNMD | | | EN | | | | |
| 142 | DRB1_ | 216 | EGRVIVSTRHE | 3 | 390 | VIVSTRH | 0.913 | 0.291185 | 3.91 | <=WB |
| | 0301 | | NNMD | | | EN | | | | |
| 142 | DRB1_ | 216 | EGRVIVSTRHE | 3 | 390 | VIVSTRH | 0.807 | 0.703699 | 1.27 | <=WB |
| | 0801 | | NNMD | | | EN | | | | |
| 142 | DRB1_ | 216 | EGRVIVSTRHE | 3 | 390 | VIVSTRH | 0.707 | 0.404824 | 2.18 | <=WB |
| | 0803 | | NNMD | | | EN | | | | |
| 142 | DRB1_ | 216 | EGRVIVSTRHE | 3 | 390 | VIVSTRH | 0.953 | 0.731689 | 1.34 | <=WB |
| | 1101 | | NNMD | | | EN | | | | |
| 143 | DRB1_ | 217 | GRVIVSTRHEN | 3 | 391 | IVSTRHE | 0.58 | 0.156019 | 4.97 | <=WB |
| | 1402 | | NMDF | | | NN | | | | |
| 154 | DRB3_ | 218 | NMDFGHSEAN | 3 | 392 | FGHSEAN | 1 | 0.193812 | 2.41 | <=WB |
| | 0202 | | TNYFK | | | TN | | | | |
| 154 | DRB1_ | 218 | NMDFGHSEAN | 3 | 392 | FGHSEAN | 0.98 | 0.448328 | 3 | <=WB |
| | 0901 | | TNYFK | | | TN | | | | |
| 154 | DRB1_ | 218 | NMDFGHSEAN | 3 | 392 | FGHSEAN | 1 | 0.506603 | 2.92 | <=WB |
| | 1001 | | TNYFK | | | TN | | | | |
| 161 | DRB1_ | 219 | EANTNYFKKA | 3 | 393 | TNYFKKA | 0.467 | 0.445901 | 4.53 | <=WB |
| | 0801 | | VEDYR | | | VE | | | | |
| 162 | DRB1_ | 220 | ANTNYFKKAV | 4 | 394 | YFKKAVE | 0.46 | 0.444984 | 4.55 | <=WB |
| | 0801 | | EDYRQ | | | DY | | | | |
| 164 | DRB5_ | 221 | TNYFKKAVED | 3 | 395 | FKKAVED | 0.96 | 0.606745 | 1.16 | <=WB |
| | 0101 | | YRQNQ | | | YR | | | | |
| 167 | DRB1_ | 222 | FKKAVEDYRQ | 4 | 396 | VEDYRQN | 1 | 0.580607 | 1.29 | <=WB |
| | 1501 | | NQLKF | | | QL | | | | |
| 167 | DRB5_ | 222 | FKKAVEDYRQ | 4 | 396 | VEDYRQN | 0.98 | 0.356492 | 2.41 | <=WB |
| | 0202 | | NQLKF | | | QL | | | | |
| 168 | DRB1_ | 223 | KKAVEDYRQN | 3 | 396 | VEDYRQN | 1 | 0.600093 | 0.58 | <=SB |
| | 1503 | | QLKFI | | | QL | | | | |
| 171 | DRB3_ | 224 | VEDYRQNQLK | 3 | 397 | YRQNQLK | 1 | 0.252481 | 1.78 | <=WB |
| | 0202 | | FIGWY | | | FI | | | | |
| 177 | DRB1_ | 225 | NQLKFIGWYSN | 4 | 398 | FIGWYSN | 1 | 0.463033 | 2.41 | <=WB |
| | 0401 | | LSEG | | | LS | | | | |
| 177 | DRB1_ | 225 | NQLKFIGWYSN | 4 | 398 | FIGWYSN | 0.993 | 0.572523 | 1.85 | <=WB |
| | 0408 | | LSEG | | | LS | | | | |
| 177 | DRB1_ | 225 | NQLKFIGWYSN | 4 | 398 | FIGWYSN | 0.993 | 0.381329 | 2.66 | <=WB |
| | 0103 | | LSEG | | | LS | | | | |
| 177 | DRB1_ | 225 | NQLKFIGWYSN | 4 | 398 | FIGWYSN | 0.927 | 0.431308 | 3.45 | <=WB |
| | 0404 | | LSEG | | | LS | | | | |
| 177 | DRB1_ | 225 | NQLKFIGWYSN | 4 | 398 | FIGWYSN | 0.9 | 0.380972 | 2.88 | <=WB |
| | 0405 | | LSEG | | | LS | | | | |
| 177 | DRB1_ | 225 | NQLKFIGWYSN | 4 | 398 | FIGWYSN | 0.98 | 0.609598 | 1.53 | <=WB |
| | 1601 | | LSEG | | | LS | | | | |
| 178 | DRB1_ | 226 | QLKFIGWYSNL | 3 | 398 | FIGWYSN | 0.68 | 0.338269 | 3.42 | <=WB |
| | 0803 | | SEGI | | | LS | | | | |
| 178 | DRB1_ | 226 | QLKFIGWYSNL | 3 | 398 | FIGWYSN | 0.613 | 0.523099 | 1.56 | <=WB |
| | 1501 | | SEGI | | | LS | | | | |
| 178 | DRB1_ | 226 | QLKFIGWYSNL | 3 | 398 | FIGWYSN | 0.66 | 0.394544 | 1.55 | <=WB |
| | 1503 | | SEGI | | | LS | | | | |
| 179 | DRB1_ | 227 | LKFIGWYSNLS | 2 | 398 | FIGWYSN | 0.6 | 0.485269 | 1.67 | <=WB |
| | 0402 | | EGIS | | | LS | | | | |
| 179 | DRB1_ | 227 | LKFIGWYSNLS | 3 | 399 | IGWYSNL | 0.82 | 0.44782 | 1.99 | <=WB |
| | 1501 | | EGIS | | | SE | | | | |
| 179 | DRB1_ | 227 | LKFIGWYSNLS | 3 | 399 | IGWYSNL | 0.64 | 0.300805 | 2.41 | <=WB |
| | 1503 | | EGIS | | | SE | | | | |
| 180 | DRB1_ | 228 | KFIGWYSNLSE | 2 | 399 | IGWYSNL | 0.593 | 0.262418 | 4.76 | <=WB |
| | 0402 | | GISA | | | SE | | | | |
| 181 | DRB3_ | 229 | FIGWYSNLSEGI | 3 | 400 | WYSNLSE | 1 | 0.652957 | 0.34 | <=SB |
| | 0202 | | SAE | | | GI | | | | |
| 181 | DRB1_ | 229 | FIGWYSNLSEGI | 3 | 400 | WYSNLSE | 0.94 | 0.398095 | 3.91 | <=WB |
| | 0901 | | SAE | | | GI | | | | |
| 181 | DRB3_ | 229 | FIGWYSNLSEGI | 3 | 400 | WYSNLSE | 0.96 | 0.126124 | 4.16 | <=WB |
| | 0101 | | SAE | | | GI | | | | |
| 188 | DRB1_ | 230 | LSEGISAEVAIR | 4 | 401 | ISAEVAIR | 1 | 0.525191 | 2.31 | <=WB |
| | 0408 | | SKQ | | | S | | | | |
| 189 | DRB1_ | 231 | SEGISAEVAIRS | 3 | 401 | ISAEVAIR | 1 | 0.650759 | 1.07 | <=WB |
| | 0401 | | KQS | | | S | | | | |
| 189 | DRB1_ | 231 | SEGISAEVAIRS | 3 | 401 | ISAEVAIR | 0.973 | 0.325196 | 4.07 | <=WB |
| | 1302 | | KQS | | | S | | | | |
| 189 | DRB1_ | 231 | SEGISAEVAIRS | 3 | 401 | ISAEVAIR | 0.993 | 0.262318 | 1.6 | <=WB |
| | 1402 | | KQS | | | S | | | | |
| 189 | DRB3_ | 231 | SEGISAEVAIRS | 3 | 401 | ISAEVAIR | 1 | 0.207415 | 2.24 | <=WB |
| | 0202 | | KQS | | | S | | | | |
| 189 | DRB5_ | 231 | SEGISAEVAIRS | 3 | 401 | ISAEVAIR | 0.993 | 0.539204 | 1.6 | <=WB |
| | 0101 | | KQS | | | S | | | | |
| 189 | DRB1_ | 231 | SEGISAEVAIRS | 3 | 401 | ISAEVAIR | 1 | 0.906074 | 0.39 | <=SB |
| | 0101 | | KQS | | | S | | | | |
| 189 | DRB1_ | 231 | SEGISAEVAIRS | 3 | 401 | ISAEVAIR | 1 | 0.966973 | 0.08 | <=SB |
| | 0102 | | KQS | | | S | | | | |
| 189 | DRB1_ | 231 | SEGISAEVAIRS | 3 | 401 | ISAEVAIR | 1 | 0.723427 | 0.55 | <=SB |
| | 0103 | | KQS | | | S | | | | |
| 189 | DRB1_ | 231 | SEGISAEVAIRS | 3 | 401 | ISAEVAIR | 1 | 0.825442 | 0.38 | <=SB |
| | 1001 | | KQS | | | S | | | | |
| 189 | DRB4_ | 231 | SEGISAEVAIRS | 3 | 401 | ISAEVAIR | 1 | 0.337983 | 0.9 | <=SB |
| | 0101 | | KQS | | | S | | | | |
| 189 | DRB4_ | 231 | SEGISAEVAIRS | 3 | 401 | ISAEVAIR | 1 | 0.337983 | 0.9 | <=SB |
| | 0103 | | KQS | | | S | | | | |
| 189 | DRB1_ | 231 | SEGISAEVAIRS | 3 | 401 | ISAEVAIR | 1 | 0.477171 | 1.73 | <=WB |
| | 0402 | | KQS | | | S | | | | |
| 189 | DRB1_ | 231 | SEGISAEVAIRS | 3 | 401 | ISAEVAIR | 1 | 0.425364 | 4.04 | <=WB |
| | 0403 | | KQS | | | S | | | | |
| 189 | DRB1_ | 231 | SEGISAEVAIRS | 3 | 401 | ISAEVAIR | 1 | 0.673794 | 1.34 | <=WB |
| | 0404 | | KQS | | | S | | | | |
| 189 | DRB1_ | 231 | SEGISAEVAIRS | 3 | 401 | ISAEVAIR | 0.987 | 0.359945 | 4.77 | <=WB |
| | 0901 | | KQS | | | S | | | | |
| 189 | DRB1_ | 231 | SEGISAEVAIRS | 3 | 401 | ISAEVAIR | 0.947 | 0.565734 | 2.71 | <=WB |
| | 1104 | | KQS | | | S | | | | |
| 189 | DRB1_ | 231 | SEGISAEVAIRS | 3 | 401 | ISAEVAIR | 0.993 | 0.290663 | 4.44 | <=WB |
| | 1201 | | KQS | | | S | | | | |
| 189 | DRB1_ | 231 | SEGISAEVAIRS | 3 | 401 | ISAEVAIR | 1 | 0.237867 | 4.21 | <=WB |
| | 1501 | | KQS | | | S | | | | |
| 189 | DRB1_ | 231 | SEGISAEVAIRS | 3 | 401 | ISAEVAIR | 1 | 0.18979 | 4.38 | <=WB |
| | 1503 | | KQS | | | S | | | | |
| 189 | DRB1_ | 231 | SEGISAEVAIRS | 3 | 401 | ISAEVAIR | 1 | 0.51072 | 2.39 | <=WB |
| | 1601 | | KQS | | | S | | | | |
| 189 | DRB5_ | 231 | SEGISAEVAIRS | 3 | 401 | ISAEVAIR | 1 | 0.485394 | 1.29 | <=WB |
| | 0202 | | KQS | | | S | | | | |
| 193 | DRB1_ | 232 | SAEVAIRSKQS | 3 | 402 | VAIRSKQ | 0.787 | 0.389284 | 2.48 | <=WB |
| | 1302 | | EKKA | | | SE | | | | |
| 193 | DRB1_ | 232 | SAEVAIRSKQS | 3 | 402 | VAIRSKQ | 0.967 | 0.494787 | 2.45 | <=WB |
| | 1303 | | EKKA | | | SE | | | | |
| 193 | DRB1_ | 232 | SAEVAIRSKQS | 3 | 402 | VAIRSKQ | 0.9 | 0.863457 | 0.28 | <=SB |
| | 1301 | | EKKA | | | SE | | | | |
| 193 | DRB1_ | 232 | SAEVAIRSKQS | 3 | 402 | VAIRSKQ | 0.953 | 0.546898 | 2.87 | <=WB |
| | 0801 | | EKKA | | | SE | | | | |
| 193 | DRB1_ | 232 | SAEVAIRSKQS | 3 | 402 | VAIRSKQ | 0.84 | 0.296531 | 4.58 | <=WB |
| | 0803 | | EKKA | | | SE | | | | |
| 193 | DRB1_ | 232 | SAEVAIRSKQS | 3 | 402 | VAIRSKQ | 0.84 | 0.532662 | 3.07 | <=WB |
| | 1104 | | EKKA | | | SE | | | | |
| 195 | DRB5_ | 233 | EVAIRSKQSEK | 3 | 403 | IRSKQSEK | 1 | 0.578159 | 1.34 | <=WB |
| | 0101 | | KAFA | | | K | | | | |
| 195 | DRB5_ | 233 | EVAIRSKQSEK | 3 | 403 | IRSKQSEK | 0.993 | 0.664463 | 0.48 | <=SB |
| | 0202 | | KAFA | | | K | | | | |
| 204 | DRB1_ | 234 | EKKAFAIIVPVY | 3 | 404 | AFAIIVPV | 0.687 | 0.292449 | 4.39 | <=WB |
| | 1201 | | SPE | | | Y | | | | |
| 205 | DRB1_ | 235 | KKAFAIIVPVYS | 3 | 405 | FAIIVPVY | 0.993 | 0.763129 | 0.57 | <=SB |
| | 0401 | | PED | | | S | | | | |
| 205 | DRB1_ | 235 | KKAFAIIVPVYS | 3 | 405 | FAIIVPVY | 0.94 | 0.330065 | 3.92 | <=WB |
| | 1302 | | PED | | | S | | | | |
| 205 | DRB1_ | 235 | KKAFAIIVPVYS | 3 | 405 | FAIIVPVY | 0.913 | 0.382895 | 3.74 | <=WB |
| | 1303 | | PED | | | S | | | | |
| 205 | DRB1_ | 235 | KKAFAIIVPVYS | 3 | 405 | FAIIVPVY | 0.993 | 0.410768 | 0.37 | <=SB |
| | 1402 | | PED | | | S | | | | |
| 205 | DRB3_ | 235 | KKAFAIIVPVYS | 3 | 405 | FAIIVPVY | 0.987 | 0.33086 | 1.27 | <=WB |
| | 0202 | | PED | | | S | | | | |
| 205 | DRB5_ | 235 | KKAFAIIVPVYS | 3 | 405 | FAIIVPVY | 1 | 0.65401 | 0.92 | <=SB |
| | 0101 | | PED | | | S | | | | |
| 205 | DRB1_ | 235 | KKAFAIIVPVYS | 3 | 405 | FAIIVPVY | 1 | 0.931901 | 0.29 | <=SB |
| | 0101 | | PED | | | S | | | | |
| 205 | DRB1_ | 235 | KKAFAIIVPVYS | 3 | 405 | FAIIVPVY | 1 | 0.790477 | 0.94 | <=SB |
| | 0102 | | PED | | | S | | | | |
| 205 | DRB1_ | 235 | KKAFAIIVPVYS | 3 | 405 | FAIIVPVY | 1 | 0.724979 | 0.55 | <=SB |
| | 0103 | | PED | | | S | | | | |
| 205 | DRB1_ | 235 | KKAFAIIVPVYS | 3 | 405 | FAIIVPVY | 0.933 | 0.617123 | 0.44 | <=SB |
| | 0803 | | PED | | | S | | | | |
| 205 | DRB1_ | 235 | KKAFAIIVPVYS | 3 | 405 | FAIIVPVY | 0.98 | 0.659578 | 0.85 | <=SB |
| | 0901 | | PED | | | S | | | | |
| 205 | DRB1_ | 235 | KKAFAIIVPVYS | 3 | 405 | FAIIVPVY | 1 | 0.959379 | 0.02 | <=SB |
| | 1001 | | PED | | | S | | | | |
| 205 | DRB1_ | 235 | KKAFAIIVPVYS | 3 | 405 | FAIIVPVY | 1 | 0.889015 | 0.49 | <=SB |
| | 1101 | | PED | | | S | | | | |
| 205 | DRB1_ | 235 | KKAFAIIVPVYS | 3 | 405 | FAIIVPVY | 1 | 0.796379 | 0.46 | <=SB |
| | 1601 | | PED | | | S | | | | |
| 205 | DRB1_ | 235 | KKAFAIIVPVYS | 3 | 405 | FAIIVPVY | 0.707 | 0.606056 | 1.78 | <=WB |
| | 0403 | | PED | | | S | | | | |
| 205 | DRB1_ | 235 | KKAFAIIVPVYS | 3 | 405 | FAIIVPVY | 0.947 | 0.395586 | 3.92 | <=WB |
| | 0404 | | PED | | | S | | | | |
| 205 | DRB1_ | 235 | KKAFAIIVPVYS | 3 | 405 | FAIIVPVY | 0.653 | 0.607022 | 1.24 | <=WB |
| | 0405 | | PED | | | S | | | | |
| 205 | DRB1_ | 235 | KKAFAIIVPVYS | 3 | 405 | FAIIVPVY | 0.993 | 0.543548 | 1.49 | <=WB |
| | 0701 | | PED | | | S | | | | |
| 205 | DRB1_ | 235 | KKAFAIIVPVYS | 3 | 405 | FAIIVPVY | 0.973 | 0.716479 | 1.17 | <=WB |
| | 0801 | | PED | | | S | | | | |
| 205 | DRB1_ | 235 | KKAFAIIVPVYS | 3 | 405 | FAIIVPVY | 0.993 | 0.503951 | 3.39 | <=WB |
| | 1104 | | PED | | | S | | | | |
| 205 | DRB4_ | 235 | KKAFAIIVPVYS | 3 | 405 | FAIIVPVY | 0.887 | 0.166278 | 2.95 | <=WB |
| | 0101 | | PED | | | S | | | | |
| 205 | DRB4_ | 235 | KKAFAIIVPVYS | 3 | 405 | FAIIVPVY | 0.887 | 0.166278 | 2.95 | <=WB |
| | 0103 | | PED | | | S | | | | |
| 206 | DRB1_ | 236 | KAFAIIVPVYSP | 2 | 405 | FAIIVPVY | 0.653 | 0.65348 | 1.27 | <=WB |
| | 0408 | | EDK | | | S | | | | |
| 206 | DRB1_ | 236 | KAFAITVPVYSP | 5 | 406 | TVPVYSPE | 0.847 | 0.351406 | 4.19 | <=WB |
| | 1303 | | EDK | | | D | | | | |
| 206 | DRB1_ | 236 | KAFAIIVPVYSP | 5 | 406 | IVPVYSPE | 0.933 | 0.849491 | 0.28 | <=SB |
| | 0403 | | EDK | | | D | | | | |
| 206 | DRB1_ | 236 | KAFAIIVPVYSP | 5 | 406 | IVPVYSPE | 0.98 | 0.896914 | 0.2 | <=SB |
| | 0405 | | EDK | | | D | | | | |
| 206 | DRB1_ | 236 | KAFAIIVPVYSP | 5 | 406 | IVPVYSPE | 0.893 | 0.515613 | 2.54 | <=WB |
| | 0404 | | EDK | | | D | | | | |
| 206 | DRB1_ | 236 | KAFAIIVPVYSP | 5 | 406 | IVPVYSPE | 0.52 | 0.537133 | 3 | <=WB |
| | 0801 | | EDK | | | D | | | | |
| 206 | DRB1_ | 236 | KAFAIIVPVYSP | 5 | 406 | IVPVYSPE | 0.573 | 0.49617 | 1.14 | <=WB |
| | 0803 | | EDK | | | D | | | | |
| 207 | DRB1_ | 237 | AFAIIVPVYSPE | 4 | 406 | IVPVYSPE | 0.907 | 0.510527 | 2.46 | <=WB |
| | 0408 | | DKL | | | D | | | | |
| 211 | DRB1_ | 238 | IVPVYSPEDKL | 4 | 407 | YSPEDKL | 1 | 0.362189 | 4.02 | <=WB |
| | 1303 | | VCGY | | | VC | | | | |
| 211 | DRB1_ | 238 | IVPVYSPEDKL | 4 | 407 | YSPEDKL | 0.973 | 0.215197 | 2.64 | <=WB |
| | 1402 | | VCGY | | | VC | | | | |
| 211 | DRB1_ | 238 | IVPVYSPEDKL | 4 | 407 | YSPEDKL | 1 | 0.50888 | 2.97 | <=WB |
| | 1101 | | VCGY | | | VC | | | | |
| 226 | DRB1_ | 239 | LAGYLLNDIVA | 3 | 408 | YLLNDIV | 1 | 0.623546 | 1.39 | <=WB |
| | 1303 | | DSFD | | | AD | | | | |
| 226 | DRB1_ | 239 | LAGYLLNDIVA | 3 | 408 | YLLNDIV | 0.807 | 0.219234 | 2.52 | <=WB |
| | 1402 | | DSFD | | | AD | | | | |
| 226 | DRB3_ | 239 | LAGYLLNDIVA | 3 | 408 | YLLNDIV | 0.993 | 0.573846 | 0.46 | <=SB |
| | 0202 | | DSFD | | | AD | | | | |
| 226 | DRB1_ | 239 | LAGYLLNDIVA | 3 | 408 | YLLNDIV | 0.487 | 0.09716 | 4.96 | <=WB |
| | 0305 | | DSFD | | | AD | | | | |
| 226 | DRB1_ | 239 | LAGYLLNDIVA | 3 | 408 | YLLNDIV | 0.64 | 0.423265 | 4.08 | <=WB |
| | 0403 | | DSFD | | | AD | | | | |
| 226 | DRB1_ | 239 | LAGYLLNDIVA | 3 | 408 | YLLNDIV | 0.973 | 0.458707 | 2.16 | <=WB |
| | 0405 | | DSFD | | | AD | | | | |
| 226 | DRB1_ | 239 | LAGYLLNDIVA | 3 | 408 | YLLNDIV | 0.987 | 0.459672 | 4.26 | <=WB |
| | 0801 | | DSFD | | | AD | | | | |
| 226 | DRB1_ | 239 | LAGYLLNDIVA | 3 | 408 | YLLNDIV | 0.987 | 0.473081 | 1.34 | <=WB |
| | 0803 | | DSFD | | | AD | | | | |
| 227 | DRB1_ | 240 | AGYLLNDIVAD | 3 | 409 | LLNDIVA | 0.84 | 0.426976 | 2.76 | <=WB |
| | 0401 | | SFDR | | | DS | | | | |
| 227 | DRB1_ | 240 | AGYLLNDIVAD | 3 | 409 | LLNDIVA | 0.98 | 0.299852 | 3.79 | <=WB |
| | 0301 | | SFDR | | | DS | | | | |
| 227 | DRB1_ | 240 | AGYLLNDIVAD | 3 | 409 | LLNDIVA | 0.66 | 0.106897 | 4.54 | <=WB |
| | 0305 | | SFDR | | | DS | | | | |
| 227 | DRB1_ | 240 | AGYLLNDIVAD | 3 | 409 | LLNDIVA | 0.593 | 0.423067 | 4.09 | <=WB |
| | 0403 | | SFDR | | | DS | | | | |
| 227 | DRB3_ | 240 | AGYLLNDIVAD | 3 | 409 | LLNDIVA | 0.647 | 0.098095 | 4.96 | <=WB |
| | 0101 | | SFDR | | | DS | | | | |
| 227 | DRB4_ | 240 | AGYLLNDIVAD | 3 | 409 | LLNDIVA | 0.673 | 0.161809 | 3.06 | <=WB |
| | 0101 | | SFDR | | | DS | | | | |
| 227 | DRB4_ | 240 | AGYLLNDIVAD | 3 | 409 | LLNDIVA | 0.673 | 0.161809 | 3.06 | <=WB |
| | 0103 | | SFDR | | | DS | | | | |
| 228 | DRB3_ | 241 | GYLLNDIVADS | 6 | 410 | IVADSFD | 0.573 | 0.133408 | 3.98 | <=WB |
| | 0101 | | FDRF | | | RF | | | | |
| 231 | DRB1_ | 242 | LNDIVADSFDR | 3 | 410 | IVADSFD | 0.993 | 0.392228 | 2.87 | <=WB |
| | 0301 | | FRFG | | | RF | | | | |
| 231 | DRB1_ | 242 | LNDIVADSFDR | 3 | 410 | IVADSFD | 0.973 | 0.196094 | 2.51 | <=WB |
| | 0305 | | FRFG | | | RF | | | | |
| 247 | DRB1_ | 243 | YKRGNFIYVDP | 5 | 411 | FIYVDPN | 0.62 | 0.386802 | 3.68 | <=WB |
| | 1303 | | NNIA | | | NI | | | | |
| 247 | DRB1_ | 243 | YKRGNFIYVDP | 5 | 411 | FIYVDPN | 0.587 | 0.299179 | 1.13 | <=WB |
| | 1402 | | NNIA | | | NI | | | | |
| 247 | DRB1_ | 243 | YKRGNFIYVDP | 5 | 411 | FIYVDPN | 0.56 | 0.371795 | 2.98 | <=WB |
| | 0405 | | NNIA | | | NI | | | | |
| 248 | DRB1_ | 244 | KRGNFIYVDPN | 4 | 411 | FIYVDPN | 0.573 | 0.680922 | 0.14 | <=SB |
| | 1302 | | NIAV | | | NI | | | | |
| 248 | DRB1_ | 244 | KRGNFIYVDPN | 5 | 412 | IYVDPNNI | 0.567 | 0.56209 | 1.83 | <=WB |
| | 1303 | | NIAV | | | A | | | | |
| 248 | DRB1_ | 244 | KRGNFIYVDPN | 5 | 412 | IYVDPNNI | 0.713 | 0.520098 | 0.11 | <=SB |
| | 1402 | | NIAV | | | A | | | | |
| 248 | DRB1_ | 244 | KRGNFIYVDPN | 4 | 411 | FIYVDPN | 0.52 | 0.589699 | 1.03 | <=WB |
| | 1401 | | NIAV | | | NI | | | | |
| 248 | DRB1_ | 244 | KRGNFIYVDPN | 4 | 411 | FIYVDPN | 0.52 | 0.589699 | 1.03 | <=WB |
| | 1454 | | NIAV | | | NI | | | | |
| 248 | DRB1_ | 244 | KRGNFIYVDPN | 4 | 411 | FIYVDPN | 0.467 | 0.298456 | 3.33 | <=WB |
| | 1501 | | NIAV | | | NI | | | | |
| 248 | DRB1_ | 244 | KRGNFIYVDPN | 5 | 412 | IYVDPNNI | 0.693 | 0.571408 | 1.42 | <=WB |
| | 0405 | | NIAV | | | A | | | | |
| 249 | DRB3_ | 245 | RGNFIYVDPNN | 3 | 411 | FIYVDPN | 0.58 | 0.693047 | 0.28 | <=SB |
| | 0202 | | IAVN | | | NI | | | | |
| 249 | DRB5_ | 245 | RGNFIYVDPNN | 3 | 411 | FIYVDPN | 0.907 | 0.683042 | 0.8 | <=SB |
| | 0101 | | IAVN | | | NI | | | | |
| 249 | DRB1_ | 245 | RGNFIYVDPNN | 4 | 412 | IYVDPNNI | 0.973 | 0.972541 | 0.02 | <=SB |
| | 0401 | | IAVN | | | A | | | | |
| 249 | DRB1_ | 245 | RGNFIYVDPNN | 4 | 412 | IYVDPNNI | 0.467 | 0.770193 | 0.04 | <=SB |
| | 1302 | | IAVN | | | A | | | | |
| 249 | DRB1_ | 245 | RGNFIYVDPNN | 3 | 411 | FIYVDPN | 1 | 0.950419 | 0.2 | <=SB |
| | 0101 | | IAVN | | | NI | | | | |
| 249 | DRB1_ | 245 | RGNFIYVDPNN | 3 | 411 | FIYVDPN | 0.927 | 0.838448 | 0.69 | <=SB |
| | 0102 | | IAVN | | | NI | | | | |
| 249 | DRB1_ | 245 | RGNFIYVDPNN | 3 | 411 | FIYVDPN | 0.98 | 0.865326 | 0.16 | <=SB |
| | 0103 | | IAVN | | | NI | | | | |
| 249 | DRB1_ | 245 | RGNFIYVDPNN | 3 | 411 | FIYVDPN | 1 | 0.957716 | 0.04 | <=SB |
| | 0701 | | IAVN | | | NI | | | | |
| 249 | DRB1_ | 245 | RGNFIYVDPNN | 3 | 411 | FIYVDPN | 0.787 | 0.681967 | 0.24 | <=SB |
| | 0803 | | IAVN | | | NI | | | | |
| 249 | DRB1_ | 245 | RGNFIYVDPNN | 3 | 411 | FIYVDPN | 0.987 | 0.856675 | 0.1 | <=SB |
| | 0901 | | IAVN | | | NI | | | | |
| 249 | DRB1_ | 245 | RGNFIYVDPNN | 3 | 411 | FIYVDPN | 0.913 | 0.86466 | 0.24 | <=SB |
| | 1001 | | IAVN | | | NI | | | | |
| 249 | DRB1_ | 245 | RGNFIYVDPNN | 3 | 411 | FIYVDPN | 0.927 | 0.885676 | 0.17 | <=SB |
| | 1601 | | IAVN | | | NI | | | | |
| 249 | DRB1_ | 245 | RGNFIYVDPNN | 4 | 412 | IYVDPNNI | 0.54 | 0.668506 | 0.6 | <=SB |
| | 1401 | | IAVN | | | A | | | | |
| 249 | DRB1_ | 245 | RGNFIYVDPNN | 4 | 412 | IYVDPNNI | 0.54 | 0.668506 | 0.6 | <=SB |
| | 1454 | | IAVN | | | A | | | | |
| 249 | DRB1_ | 245 | RGNFIYVDPNN | 3 | 411 | FIYVDPN | 0.867 | 0.592006 | 2.33 | <=WB |
| | 0801 | | IAVN | | | NI | | | | |
| 249 | DRB1_ | 245 | RGNFIYVDPNN | 3 | 411 | FIYVDPN | 0.753 | 0.494451 | 3.13 | <=WB |
| | 1101 | | IAVN | | | NI | | | | |
| 249 | DRB1_ | 245 | RGNFIYVDPNN | 3 | 411 | FIYVDPN | 0.607 | 0.377229 | 2.7 | <=WB |
| | 1201 | | IAVN | | | NI | | | | |
| 249 | DRB1_ | 245 | RGNFIYVDPNN | 3 | 411 | FIYVDPN | 0.547 | 0.340044 | 1.98 | <=WB |
| | 1503 | | IAVN | | | NI | | | | |
| 249 | DRB1_ | 245 | RGNFIYVDPNN | 1 | 412 | IYVDPNNI | 0.527 | 0.414469 | 2.25 | <=WB |
| | 1501 | | IAVN | | | A | | | | |
| 249 | DRB5_ | 245 | RGNFIYVDPNN | 4 | 412 | IYVDPNNI | 0.587 | 0.388662 | 2.07 | <=WB |
| | 0202 | | IAVN | | | A | | | | |
| 250 | DRB1_ | 246 | GNFIYVDPNNI | 3 | 412 | IYVDPNNI | 0.967 | 0.936355 | 0.08 | <=SB |
| | 0408 | | AVNP | | | A | | | | |
| 250 | DRB3_ | 246 | GNFIYVDPNNI | 3 | 412 | IYVDPNNI | 0.793 | 0.661833 | 0.32 | <=SB |
| | 0202 | | AVNP | | | A | | | | |
| 250 | DRB1_ | 246 | GNFIYVDPNNI | 3 | 412 | IYVDPNNI | 1 | 0.934593 | 0.13 | <=SB |
| | 0301 | | AVNP | | | A | | | | |
| 250 | DRB1_ | 246 | GNFIYVDPNNI | 3 | 412 | IYVDPNNI | 0.987 | 0.765373 | 0.06 | <=SB |
| | 0305 | | AVNP | | | A | | | | |
| 250 | DRB1_ | 246 | GNFIYVDPNNI | 3 | 412 | IYVDPNNI | 1 | 0.672314 | 0.64 | <=SB |
| | 0402 | | AVNP | | | A | | | | |
| 250 | DRB1_ | 246 | GNFIYVDPNNI | 3 | 412 | IYVDPNNI | 0.993 | 0.8967 | 0.14 | <=SB |
| | 0403 | | AVNP | | | A | | | | |
| 250 | DRB1_ | 246 | GNFIYVDPNNI | 3 | 412 | IYVDPNNI | 1 | 0.938004 | 0.11 | <=SB |
| | 0404 | | AVNP | | | A | | | | |
| 250 | DRB1_ | 246 | GNFIYVDPNNI | 3 | 412 | IYVDPNNI | 0.493 | 0.550614 | 0.76 | <=SB |
| | 0803 | | AVNP | | | A | | | | |
| 250 | DRB3_ | 246 | GNFIYVDPNNI | 3 | 412 | IYVDPNNI | 1 | 0.901495 | 0.07 | <=SB |
| | 0101 | | AVNP | | | A | | | | |
| 250 | DRB4_ | 246 | GNFIYVDPNNI | 3 | 412 | IYVDPNNI | 0.973 | 0.515958 | 0.35 | <=SB |
| | 0101 | | AVNP | | | A | | | | |
| 250 | DRB4_ | 246 | GNFIYVDPNNI | 3 | 412 | IYVDPNNI | 0.973 | 0.515958 | 0.35 | <=SB |
| | 0103 | | AVNP | | | A | | | | |
| 250 | DRB1_ | 246 | GNFIYVDPNNI | 3 | 412 | IYVDPNNI | 0.927 | 0.419872 | 4.51 | <=WB |
| | 1104 | | AVNP | | | A | | | | |
| 250 | DRB1_ | 246 | GNFIYVDPNNI | 3 | 412 | IYVDPNNI | 0.627 | 0.316006 | 3.83 | <=WB |
| | 1201 | | AVNP | | | A | | | | |
| 250 | DRB1_ | 246 | GNFIYVDPNNI | 3 | 412 | IYVDPNNI | 0.9 | 0.530695 | 3.2 | <=WB |
| | 1301 | | AVNP | | | A | | | | |
| 250 | DRB1_ | 246 | GNFIYVDPNNI | 3 | 412 | IYVDPNNI | 0.753 | 0.278949 | 2.69 | <=WB |
| | 1503 | | AVNP | | | A | | | | |
| 252 | DRB3_ | 247 | FIYVDPNNIAV | 3 | 413 | VDPNNIA | 0.493 | 0.154734 | 3.04 | <=WB |
| | 0202 | | NPFE | | | VN | | | | |
| 256 | DRB1_ | 248 | DPNNIAVNPFE | 4 | 414 | IAVNPFEE | 0.987 | 0.39614 | 2.35 | <=WB |
| | 1302 | | EYNE | | | Y | | | | |
| 257 | DRB1_ | 249 | PNNIAVNPFEE | 3 | 414 | IAVNPFEE | 0.913 | 0.106737 | 4.55 | <=WB |
| | 0305 | | YNET | | | Y | | | | |
| 257 | DRB1_ | 249 | PNNIAVNPFEE | 3 | 414 | IAVNPFEE | 0.88 | 0.33157 | 3.48 | <=WB |
| | 1201 | | YNET | | | Y | | | | |
| 262 | DRB5_ | 250 | VNPFEEYNETS | 3 | 415 | FEEYNET | 0.987 | 0.345048 | 2.54 | <=WB |
| | 0202 | | RVSS | | | SR | | | | |
| 264 | DRB1_ | 251 | PFEEYNETSRV | 4 | 416 | YNETSRV | 1 | 0.524341 | 2.16 | <=WB |
| | 1303 | | SSKF | | | SS | | | | |
| 264 | DRB1_ | 251 | PFEEYNETSRV | 4 | 416 | YNETSRV | 1 | 0.816527 | 0.87 | <=SB |
| | 1101 | | SSKF | | | SS | | | | |
| 264 | DRB1_ | 251 | PFEEYNETSRV | 4 | 416 | YNETSRV | 0.993 | 0.526089 | 3.17 | <=WB |
| | 0801 | | SSKF | | | SS | | | | |
| 275 | DRB1_ | 252 | SSKFLNVLKDV | 3 | 417 | FLNVLKD | 0.953 | 0.344776 | 4.29 | <=WB |
| | 1303 | | FSKP | | | VF | | | | |
| 275 | DRB1_ | 252 | SSKFLNVLKDV | 3 | 417 | FLNVLKD | 0.94 | 0.489358 | 1.83 | <=WB |
| | 1401 | | FSKP | | | VF | | | | |
| 275 | DRB1_ | 252 | SSKFLNVLKDV | 3 | 417 | FLNVLKD | 0.94 | 0.489358 | 1.83 | <=WB |
| | 1454 | | FSKP | | | VF | | | | |
| 275 | DRB1_ | 252 | SSKFLNVLKDV | 4 | 418 | LNVLKDV | 0.527 | 0.332566 | 3.46 | <=WB |
| | 1201 | | FSKP | | | FS | | | | |
| 276 | DRB1_ | 253 | SKFLNVLKDVF | 3 | 418 | LNVLKDV | 1 | 0.502311 | 2.09 | <=WB |
| | 0401 | | SKPP | | | FS | | | | |
| 276 | DRB1_ | 253 | SKFLNVLKDVF | 3 | 418 | LNVLKDV | 1 | 0.761161 | 0.67 | <=SB |
| | 0408 | | SKPP | | | FS | | | | |
| 276 | DRB1_ | 253 | SKFLNVLKDVF | 3 | 418 | LNVLKDV | 1 | 0.70941 | 0.5 | <=SB |
| | 0402 | | SKPP | | | FS | | | | |
| 276 | DRB1_ | 253 | SKFLNVLKDVF | 3 | 418 | LNVLKDV | 1 | 0.715001 | 0.97 | <=SB |
| | 0403 | | SKPP | | | FS | | | | |
| 276 | DRB1_ | 253 | SKFLNVLKDVF | 3 | 418 | LNVLKDV | 1 | 0.940804 | 0.1 | <=SB |
| | 0404 | | SKPP | | | FS | | | | |
| 276 | DRB1_ | 253 | SKFLNVLKDVF | 3 | 418 | LNVLKDV | 1 | 0.551836 | 2.56 | <=WB |
| | 0102 | | SKPP | | | FS | | | | |
| 276 | DRB1_ | 253 | SKFLNVLKDVF | 3 | 418 | LNVLKDV | 1 | 0.44794 | 2.25 | <=WB |
| | 0405 | | SKPP | | | FS | | | | |
| 276 | DRB1_ | 253 | SKFLNVLKDVF | 3 | 418 | LNVLKDV | 0.993 | 0.479425 | 3.29 | <=WB |
| | 1001 | | SKPP | | | FS | | | | |
| 276 | DRB1_ | 253 | SKFLNVLKDVF | 3 | 418 | LNVLKDV | 0.953 | 0.469232 | 3.83 | <=WB |
| | 1104 | | SKPP | | | FS | | | | |
| 276 | DRB1_ | 253 | SKFLNVLKDVF | 3 | 418 | LNVLKDV | 0.993 | 0.265083 | 3.76 | <=WB |
| | 1501 | | SKPP | | | FS | | | | |
| 276 | DRB1_ | 253 | SKFLNVLKDVF | 3 | 418 | LNVLKDV | 0.973 | 0.195386 | 4.23 | <=WB |
| | 1503 | | SKPP | | | FS | | | | |
| 276 | DRB1_ | 253 | SKFLNVLKDVF | 3 | 418 | LNVLKDV | 0.993 | 0.397175 | 3.77 | <=WB |
| | 1601 | | SKPP | | | FS | | | | |
| 276 | DRB4_ | 253 | SKFLNVLKDVF | 3 | 418 | LNVLKDV | 0.953 | 0.17341 | 2.8 | <=WB |
| | 0101 | | SKPP | | | FS | | | | |
| 276 | DRB4_ | 253 | SKFLNVLKDVF | 3 | 418 | LNVLKDV | 0.953 | 0.17341 | 2.8 | <=WB |
| | 0103 | | SKPP | | | FS | | | | |
| 288 | DRB3_ | 254 | KPPFPSNIASEV | 3 | 419 | FPSNIASE | 1 | 0.188302 | 2.48 | <=WB |
| | 0202 | | SVY | | | V | | | | |
| 292 | DRB1_ | 255 | PSNIASEVSVYT | 3 | 420 | IASEVSV | 1 | 0.439254 | 2.63 | <=WB |
| | 0401 | | IDR | | | YT | | | | |
| 292 | DRB1_ | 255 | PSNIASEVSVYT | 3 | 420 | IASEVSV | 1 | 0.379193 | 3.98 | <=WB |
| | 0408 | | IDR | | | YT | | | | |
| 292 | DRB1_ | 255 | PSNIASEVSVYT | 3 | 420 | IASEVSV | 1 | 0.86104 | 0.56 | <=SB |
| | 0102 | | IDR | | | YT | | | | |
| 292 | DRB1_ | 255 | PSNIASEVSVYT | 3 | 420 | ASEVSV | 1 | 0.673158 | 1.49 | <=WB |
| | 0101 | | IDR | | | YT | | | | |
| 292 | DRB1_ | 255 | PSNIASEVSVYT | 3 | 420 | IASEVSV | 1 | 0.484962 | 2.85 | <=WB |
| | 0404 | | IDR | | | YT | | | | |
| 292 | DRB1_ | 255 | PSNIASEVSVYT | 3 | 420 | IASEVSV | 1 | 0.456291 | 3.64 | <=WB |
| | 1001 | | IDR | | | YT | | | | |
| 292 | DRB1_ | 255 | PSNIASEVSVYT | 3 | 420 | IASEVSV | 1 | 0.228946 | 4.39 | <=WB |
| | 1501 | | IDR | | | YT | | | | |
| 296 | DRB1_ | 256 | ASEVSVYTIDRI | 3 | 421 | VSVYTID | 0.92 | 0.210123 | 4.77 | <=WB |
| | 1501 | | LLS | | | RI | | | | |
| 297 | DRB1_ | 257 | SEVSVYTIDRIL | 4 | 422 | VYTIDRIL | 0.6 | 0.297773 | 4.97 | <=WB |
| | 1302 | | LSE | | | L | | | | |
| 297 | DRB1_ | 257 | SEVSVYTIDRIL | 4 | 422 | VYTIDRIL | 0.987 | 0.782516 | 0.24 | <=SB |
| | 1401 | | LSE | | | L | | | | |
| 297 | DRB1_ | 257 | SEVSVYTIDRIL | 4 | 422 | VYTIDRIL | 0.987 | 0.782516 | 0.24 | <=SB |
| | 1454 | | LSE | | | L | | | | |
| 297 | DRB1_ | 257 | SEVSVYTIDRIL | 4 | 422 | VYTIDRIL | 0.853 | 0.292523 | 4.39 | <=WB |
| | 1201 | | LSE | | | L | | | | |
| 298 | DRB1_ | 258 | EVSVYTIDRILL | 4 | 423 | YTIDRILL | 0.793 | 0.344757 | 4.29 | <=WB |
| | 1303 | | SEM | | | S | | | | |
| 299 | DRB1_ | 259 | VSVYTIDRILLS | 3 | 423 | YTIDRILL | 1 | 0.706954 | 0.8 | <=SB |
| | 0401 | | EMG | | | S | | | | |
| 299 | DRB1_ | 259 | VSVYTIDRILLS | 3 | 423 | YTIDRILL | 1 | 0.592941 | 1.69 | <=WB |
| | 0408 | | EMG | | | S | | | | |
| 299 | DRB1_ | 259 | VSVYTIDRILLS | 3 | 423 | YTIDRILL | 0.873 | 0.341932 | 0.74 | <=SB |
| | 1402 | | EMG | | | S | | | | |
| 299 | DRB3_ | 259 | VSVYTIDRILLS | 3 | 423 | YTIDRILL | 0.987 | 0.240639 | 1.89 | <=WB |
| | 0202 | | EMG | | | S | | | | |
| 299 | DRB1_ | 259 | VSVYTIDRILLS | 3 | 423 | YTIDRILL | 1 | 0.463289 | 0.64 | <=SB |
| | 0305 | | EMG | | | S | | | | |
| 299 | DRB3_ | 259 | VSVYTIDRILLS | 3 | 423 | YTIDRILL | 1 | 0.497895 | 0.92 | <=SB |
| | 0101 | | EMG | | | S | | | | |
| 299 | DRB1_ | 259 | VSVYTIDRILLS | 3 | 423 | YTIDRILL | 0.987 | 0.389487 | 2.9 | <=WB |
| | 0301 | | EMG | | | S | | | | |
| 299 | DRB1_ | 259 | VSVYTIDRILLS | 3 | 423 | YTIDRILL | 1 | 0.27035 | 4.5 | <=WB |
| | 0405 | | EMG | | | S | | | | |
| 299 | DRB1_ | 259 | VSVYTIDRILLS | 3 | 423 | YTIDRILL | 0.847 | 0.302475 | 4.4 | <=WB |
| | 0803 | | EMG | | | S | | | | |
| 299 | DRB1_ | 259 | VSVYTIDRILLS | 3 | 423 | YTIDRILL | 1 | 0.429018 | 4.1 | <=WB |
| | 1001 | | EMG | | | S | | | | |
| 299 | DRB4_ | 259 | VSVYTIDRILLS | 3 | 423 | YTIDRILL | 0.947 | 0.116045 | 4.77 | <=WB |
| | 0101 | | EMG | | | S | | | | |
| 299 | DRB4_ | 259 | VSVYTIDRILLS | 3 | 423 | YTIDRILL | 0.947 | 0.116045 | 4.77 | <=WB |
| | 0103 | | EMG | | | S | | | | |
| 314 | DRB1_ | 260 | EDCYYAMLPIS | 3 | 424 | YYAMLPI | 0.993 | 0.278347 | 4.95 | <=WB |
| | 0401 | | SKLG | | | SS | | | | |
| 314 | DRB1_ | 260 | EDCYYAMLPIS | 3 | 424 | YYAMLPI | 1 | 0.372538 | 4.09 | <=WB |
| | 0408 | | SKLG | | | SS | | | | |
| 314 | DRB1_ | 260 | EDCYYAMLPIS | 3 | 424 | YYAMLPI | 1 | 0.872453 | 0.22 | <=SB |
| | 1001 | | SKLG | | | SS | | | | |
| 314 | DRB1_ | 260 | EDCYYAMLPIS | 3 | 424 | YYAMLPI | 1 | 0.604008 | 1.85 | <=WB |
| | 0101 | | SKLG | | | SS | | | | |
| 314 | DRB1_ | 260 | EDCYYAMLPIS | 3 | 424 | YYAMLPI | 0.967 | 0.321634 | 3.45 | <=WB |
| | 0103 | | SKLG | | | SS | | | | |
| 317 | DRB1_ | 261 | YYAMLPISSKL | 3 | 425 | MLPISSKL | 1 | 0.520137 | 1.42 | <=WB |
| | 0402 | | GEKS | | | G | | | | |
| 317 | DRB1_ | 261 | YYAMLPISSKL | 3 | 425 | MLPISSKL | 1 | 0.476271 | 2.94 | <=WB |
| | 0404 | | GEKS | | | G | | | | |
| 317 | DRB1_ | 261 | YYAMLPISSKL | 3 | 425 | MLPISSKL | 0.987 | 0.417536 | 4.55 | <=WB |
| | 1104 | | GEKS | | | G | | | | |
| 323 | DRB1_ | 262 | ISSKLGEKSGVL | 4 | 426 | LGEKSGV | 0.993 | 0.282872 | 4.08 | <=WB |
| | 0103 | | IAR | | | LI | | | | |
| 328 | DRB1_ | 263 | GEKSGVLIARL | 4 | 427 | GVLIARL | 0.807 | 0.439648 | 4.75 | <=WB |
| | 1301 | | PYKD | | | PY | | | | |
| 329 | DRB1_ | 264 | EKSGVLIARLP | 3 | 427 | GVLIARL | 0.613 | 0.32288 | 3.67 | <=WB |
| | 1201 | | YKDI | | | PY | | | | |
| 330 | DRB1_ | 265 | KSGVLIARLPY | 5 | 428 | IARLPYK | 0.553 | 0.330858 | 3.5 | <=WB |
| | 1201 | | KDIY | | | DI | | | | |
| 331 | DRB1_ | 266 | SGVLIARLPYK | 3 | 429 | LIARLPY | 0.94 | 0.49446 | 3.66 | <=WB |
| | 0801 | | DIYG | | | KD | | | | |
| 336 | DRB1_ | 267 | ARLPYKDIYGV | 4 | 430 | YKDIYGV | 1 | 0.494127 | 3.08 | <=WB |
| | 1001 | | ISSL | | | IS | | | | |
| 337 | DRB1_ | 268 | RLPYKDIYGVIS | 3 | 430 | YKDIYGV | 1 | 0.628633 | 1.72 | <=WB |
| | 0101 | | SLR | | | IS | | | | |
| 337 | DRB1_ | 268 | RLPYKDIYGVIS | 3 | 430 | YKDIYGV | 0.993 | 0.284238 | 4.05 | <=WB |
| | 0103 | | SLR | | | IS | | | | |
| 337 | DRB1_ | 268 | RLPYKDIYGVIS | 3 | 430 | YKDIYGV | 0.953 | 0.409906 | 3.6 | <=WB |
| | 1601 | | SLR | | | IS | | | | |
| 339 | DRB1_ | 269 | PYKDIYGVISSL | 4 | 431 | IYGVISSL | 0.527 | 0.454252 | 3.04 | <=WB |
| | 1601 | | RFQ | | | R | | | | |
| 340 | DRB5_ | 270 | YKDIYGVISSLR | 3 | 431 | IYGVISSL | 0.693 | 0.604268 | 1.17 | <=WB |
| | 0101 | | FQY | | | R | | | | |
| 340 | DRB5_ | 270 | YKDIYGVISSLR | 3 | 431 | IYGVISSL | 0.96 | 0.638962 | 0.56 | <=SB |
| | 0202 | | FQY | | | R | | | | |
| 340 | DRB1_ | 270 | YKDIYGVISSLR | 4 | 432 | YGVISSLR | 0.967 | 0.640478 | 0.95 | <=SB |
| | 0701 | | FQY | | | F | | | | |
| 340 | DRB1_ | 270 | YKDIYGVISSLR | 4 | 432 | YGVISSLR | 0.933 | 0.545754 | 1.72 | <=WB |
| | 0901 | | FQY | | | F | | | | |
| 340 | DRB1_ | 270 | YKDIYGVISSLR | 4 | 432 | YGVISSLR | 0.787 | 0.460303 | 1.68 | <=WB |
| | 1201 | | FQY | | | F | | | | |
| 340 | DRB1_ | 270 | YKDIYGVISSLR | 4 | 432 | YGVISSLR | 0.527 | 0.348306 | 3.94 | <=WB |
| | 1401 | | FQY | | | F | | | | |
| 340 | DRB1_ | 270 | YKDIYGVISSLR | 4 | 432 | YGVISSLR | 0.527 | 0.348306 | 3.94 | <=WB |
| | 1454 | | FQY | | | F | | | | |
| 340 | DRB1_ | 270 | YKDIYGVISSLR | 4 | 432 | YGVISSLR | 0.62 | 0.587475 | 1.71 | <=WB |
| | 1601 | | FQY | | | F | | | | |
| 340 | DRB3_ | 270 | YKDIYGVISSLR | 4 | 432 | YGVISSLR | 0.94 | 0.107599 | 4.66 | <=WB |
| | 0101 | | FQY | | | F | | | | |
| 341 | DRB1_ | 271 | KDIYGVISSLRF | 5 | 433 | VISSLRFQ | 0.4 | 0.375842 | 3.38 | <=WB |
| | 1401 | | QYI | | | Y | | | | |
| 341 | DRB1_ | 271 | KDIYGVISSLRF | 5 | 433 | VISSLRFQ | 0.4 | 0.375842 | 3.38 | <=WB |
| | 1454 | | QYI | | | Y | | | | |
| 347 | DRB4_ | 272 | ISSLRFQYILYS | 3 | 434 | LRFQYIL | 0.973 | 0.179631 | 2.66 | <=WB |
| | 0101 | | VLG | | | YS | | | | |
| 347 | DRB4_ | 272 | ISSLRFQYILYS | 3 | 434 | LRFQYIL | 0.973 | 0.179631 | 2.66 | <=WB |
| | 0103 | | VLG | | | YS | | | | |
| 361 | DRB1_ | 273 | GIIALSIVLSIRI | 4 | 435 | LSIVLSIRI | 0.953 | 0.30028 | 3.88 | <=WB |
| | 0701 | | DR | | | | | | | |
| 364 | DRB1_ | 274 | ALSIVLSIRIDRI | 3 | 436 | IVLSIRID | 0.993 | 0.371101 | 3.06 | <=WB |
| | 0301 | | IS | | | R | | | | |
| 364 | DRB1_ | 274 | ALSIVLSIRIDRI | 3 | 436 | IVLSIRID | 0.913 | 0.502146 | 3.64 | <=WB |
| | 1301 | | IS | | | R | | | | |
| 364 | DRB4_ | 274 | ALSIVLSIRIDRI | 4 | 437 | VLSIRIDR | 0.987 | 0.204925 | 2.18 | <=WB |
| | 0101 | | IS | | | I | | | | |
| 364 | DRB4_ | 274 | ALSIVLSIRIDRI | 4 | 437 | VLSIRIDR | 0.987 | 0.204925 | 2.18 | <=WB |
| | 0103 | | IS | | | I | | | | |
| 365 | DRB1_ | 275 | LSIVLSIRIDRIIS | 6 | 438 | IRIDRIISF | 0.78 | 0.426994 | 2.57 | <=WB |
| | 0301 | | F | | | | | | | |
| 367 | DRB1_ | 276 | IVLSIRIDRIISFR | 4 | 438 | IRIDRIISF | 0.92 | 0.220938 | 2.48 | <=WB |
| | 1402 | | L | | | | | | | |
| 367 | DRB1_ | 276 | IVLSIRIDRIISFR | 4 | 438 | IRIDRIISF | 0.893 | 0.323006 | 4.54 | <=WB |
| | 1401 | | L | | | | | | | |
| 367 | DRB1_ | 276 | IVLSIRIDRIISFR | 4 | 438 | IRIDRIISF | 0.893 | 0.323006 | 4.54 | <=WB |
| | 1454 | | L | | | | | | | |
| 368 | DRB1_ | 277 | VLSIRIDRIISFR | 3 | 438 | IRIDRIISF | 0.9 | 0.472188 | 1.31 | <=WB |
| | 1302 | | LN | | | | | | | |
| 368 | DRB1_ | 277 | VLSIRIDRIISFR | 3 | 438 | IRIDRIISF | 1 | 0.472044 | 0.61 | <=SB |
| | 0305 | | LN | | | | | | | |
| 368 | DRB3_ | 277 | VLSIRIDRIISFR | 3 | 438 | IRIDRIISF | 1 | 0.681139 | 0.43 | <=SB |
| | 0101 | | LN | | | | | | | |
| 368 | DRB1_ | 277 | VLSIRIDRIISFR | 3 | 438 | TRIDRIISF | 0.56 | 0.510954 | 1.28 | <=WB |
| | 1201 | | LN | | | | | | | |
| 369 | DRB1_ | 278 | LSIRIDRIISFRL | 4 | 439 | IDRIISFRL | 0.973 | 0.411975 | 4.04 | <=WB |
| | 0102 | | NA | | | | | | | |
| 369 | DRB1_ | 278 | LSIRIDRIISFRL | 4 | 439 | IDRIISFRL | 0.98 | 0.267099 | 4.43 | <=WB |
| | 0701 | | NA | | | | | | | |
| 369 | DRB1_ | 278 | LSIRIDRIISFRL | 4 | 439 | IDRIISFRL | 0.713 | 0.505845 | 1.32 | <=WB |
| | 1201 | | NA | | | | | | | |
| 369 | DRB1_ | 278 | LSIRIDRIISFRL | 4 | 439 | IDRIISFRL | 1 | 0.600582 | 1.61 | <=WB |
| | 1601 | | NA | | | | | | | |
| 369 | DRB5_ | 278 | LSIRIDRIISFRL | 4 | 439 | IDRIISFRL | 0.82 | 0.252358 | 4.11 | <=WB |
| | 0202 | | NA | | | | | | | |
| 370 | DRB1_ | 279 | SIRIDRIISFRLN | 3 | 439 | IDRIISFRL | 0.9 | 0.425642 | 1.35 | <=WB |
| | 1503 | | AI | | | | | | | |
| 371 | DRB1_ | 280 | IRIDRIISFRLNA | 2 | 439 | IDRIISFRL | 0.673 | 0.477405 | 1.81 | <=WB |
| | 1501 | | IR | | | | | | | |
| 371 | DRB1_ | 280 | IRIDRIISFRLNA | 5 | 440 | IISFRLNAI | 0.52 | 0.375566 | 1.68 | <=WB |
| | 1503 | | IR | | | | | | | |
| 372 | DRB1_ | 281 | RIDRIISFRLNAI | 4 | 440 | IISFRLNAI | 0.847 | 0.404849 | 2.31 | <=WB |
| | 1201 | | RV | | | | | | | |
| 372 | DRB1_ | 281 | RIDRIISFRLNAI | 4 | 440 | IISFRLNAI | 0.8 | 0.334601 | 2.93 | <=WB |
| | 1501 | | RV | | | | | | | |
| 372 | DRB1_ | 281 | RIDRIISFRLNAI | 5 | 441 | ISFRLNAI | 0.593 | 0.458978 | 4.39 | <=WB |
| | 1301 | | RV | | | R | | | | |
| 372 | DRB5_ | 281 | RIDRIISFRLNAI | 5 | 441 | ISFRLNAI | 0.62 | 0.359212 | 2.38 | <=WB |
| | 0202 | | RV | | | R | | | | |
| 376 | DRB1_ | 282 | IISFRLNAIRVL | 3 | 442 | FRLNAIR | 0.973 | 0.438872 | 1.7 | <=WB |
| | 1302 | | VQD | | | VL | | | | |
| 376 | DRB1_ | 282 | IISFRLNAIRVL | 3 | 442 | FRLNAIR | 0.767 | 0.171415 | 4.18 | <=WB |
| | 1402 | | VQD | | | VL | | | | |
| 376 | DRB3_ | 282 | IISFRLNAIRVL | 3 | 442 | FRLNAIR | 1 | 0.549091 | 0.49 | <=SB |
| | 0202 | | VQD | | | VL | | | | |
| 381 | DRB1_ | 283 | LNAIRVLVQDM | 3 | 443 | IRVLVQD | 0.993 | 0.766392 | 0.22 | <=SB |
| | 1201 | | VKGN | | | MV | | | | |
| 381 | DRB1_ | 283 | LNAIRVLVQDM | 3 | 443 | IRVLVQD | 0.96 | 0.359439 | 3.71 | <=WB |
| | 1454 | | VKGN | | | MV | | | | |
| 382 | DRB4_ | 284 | NAIRVLVQDM | 2 | 443 | IRVLVQD | 0.62 | 0.313831 | 1.04 | <=WB |
| | 0101 | | VKGNL | | | MV | | | | |
| 382 | DRB4_ | 284 | NAIRVLVQDM | 2 | 443 | IRVLVQD | 0.62 | 0.313831 | 1.04 | <=WB |
| | 0103 | | VKGNL | | | MV | | | | |
| 383 | DRB4_ | 285 | AIRVLVQDMV | 3 | 444 | VLVQDM | 0.893 | 0.305328 | 1.09 | <=WB |
| | 0101 | | KGNLD | | | VKG | | | | |
| 383 | DRB4_ | 285 | AIRVLVQDMV | 3 | 444 | VLVQDM | 0.893 | 0.305328 | 1.09 | <=WB |
| | 0103 | | KGNLD | | | VKG | | | | |
| 385 | DRB1_ | 286 | RVLVQDMVKG | 3 | 445 | VQDMVK | 1 | 0.502265 | 3.41 | <=WB |
| | 1104 | | NLDKD | | | GNL | | | | |
| 385 | DRB1_ | 286 | RVLVQDMVKG | 3 | 445 | VQDMVK | 0.927 | 0.270951 | 4.98 | <=WB |
| | 1201 | | NLDKD | | | GNL | | | | |
| 385 | DRB1_ | 286 | RVLVQDMVKG | 3 | 445 | VQDMVK | 0.967 | 0.485124 | 1.87 | <=WB |
| | 1401 | | NLDKD | | | GNL | | | | |
| 385 | DRB1_ | 286 | RVLVQDMVKG | 3 | 445 | VQDMVK | 0.967 | 0.485124 | 1.87 | <=WB |
| | 1454 | | NLDKD | | | GNL | | | | |
| 387 | DRB4_ | 287 | LVQDMVKGNL | 4 | 446 | MVKGNL | 0.873 | 0.119592 | 4.57 | <=WB |
| | 0101 | | DKDYA | | | DKD | | | | |
| 387 | DRB4_ | 287 | LVQDMVKGNL | 4 | 446 | MVKGNL | 0.873 | 0.119592 | 4.57 | <=WB |
| | 0103 | | DKDYA | | | DKD | | | | |
| 393 | DRB1_ | 288 | KGNLDKDYAL | 3 | 447 | LDKDYAL | 0.993 | 0.340332 | 4.36 | <=WB |
| | 1303 | | DDDEN | | | DD | | | | |
| 393 | DRB1_ | 288 | KGNLDKDYAL | 3 | 447 | LDKDYAL | 0.98 | 0.13989 | 3.55 | <=WB |
| | 0305 | | DDDEN | | | DD | | | | |
| 393 | DRB3_ | 288 | KGNLDKDYAL | 3 | 447 | LDKDYAL | 0.987 | 0.217099 | 2.68 | <=WB |
| | 0101 | | DDDEN | | | DD | | | | |
| 394 | DRB3_ | 289 | GNLDKDYALD | 6 | 448 | YALDDDE | 0.673 | 0.179029 | 3.16 | <=WB |
| | 0101 | | DDENT | | | NT | | | | |
| 397 | DRB1_ | 290 | DKDYALDDDE | 3 | 448 | YALDDDE | 1 | 0.846945 | 0.3 | <=SB |
| | 0401 | | NTLDE | | | NT | | | | |
| 397 | DRB1_ | 290 | DKDYALDDDE | 3 | 448 | YALDDDE | 1 | 0.655181 | 1.26 | <=WB |
| | 0408 | | NTLDE | | | NT | | | | |
| 397 | DRB3_ | 290 | DKDYALDDDE | 3 | 448 | YALDDDE | 0.973 | 0.158108 | 2.97 | <=WB |
| | 0202 | | NTLDE | | | NT | | | | |
| 397 | DRB1_ | 290 | DKDYALDDDE | 3 | 448 | YALDDDE | 0.94 | 0.299521 | 1.46 | <=WB |
| | 0305 | | NTLDE | | | NT | | | | |
| 397 | DRB1_ | 290 | DKDYALDDDE | 3 | 448 | YALDDDE | 0.86 | 0.389247 | 4.71 | <=WB |
| | 0403 | | NTLDE | | | NT | | | | |
| 397 | DRB1_ | 290 | DKDYALDDDE | 3 | 448 | YALDDDE | 0.933 | 0.398896 | 2.7 | <=WB |
| | 0405 | | NTLDE | | | NT | | | | |
| 410 | DRB1_ | 291 | DELGMLSLQV | 4 | 449 | MLSLQVV | 0.853 | 0.431583 | 1.97 | <=WB |
| | 1201 | | VKMKK | | | KM | | | | |
| 411 | DRB1_ | 292 | ELGMLSLQVV | 3 | 449 | MLSLQVV | 0.993 | 0.315414 | 4.31 | <=WB |
| | 0101 | | KMKKA | | | KM | | | | |
| 411 | DRB1_ | 292 | ELGMLSLQVV | 3 | 449 | MLSLQVV | 1 | 0.55928 | 2.49 | <=WB |
| | 0102 | | KMKKA | | | KM | | | | |
| 411 | DRB1_ | 292 | ELGMLSLQVV | 3 | 449 | MLSLQVV | 0.987 | 0.465243 | 3.5 | <=WB |
| | 1001 | | KMKKA | | | KM | | | | |
| 415 | DRB1_ | 293 | LSLQVVKMKK | 4 | 450 | VVKMKK | 0.593 | 0.352955 | 3.27 | <=WB |
| | 1302 | | AISVA | | | AIS | | | | |
| 415 | DRB1_ | 293 | LSLQVVKMKK | 4 | 450 | VVKMKK | 0.993 | 0.312947 | 4.83 | <=WB |
| | 1303 | | AISVA | | | AIS | | | | |
| 415 | DRB1_ | 293 | LSLQVVKMKK | 4 | 450 | VVKMKK | 0.927 | 0.222505 | 2.44 | <=WB |
| | 1402 | | AISVA | | | AIS | | | | |
| 415 | DRB1_ | 293 | LSLQVVKMKK | 4 | 450 | VVKMKK | 1 | 0.889617 | 0.49 | <=SB |
| | 1101 | | AISVA | | | AIS | | | | |
| 415 | DRB1_ | 293 | LSLQVVKMKK | 1 | 450 | VVKMKK | 1 | 0.953862 | 0.13 | <=SB |
| | 1104 | | AISVA | | | AIS | | | | |
| 415 | DRB1_ | 293 | LSLQVVKMKK | 4 | 450 | VVKMKK | 0.94 | 0.593419 | 2.31 | <=WB |
| | 0801 | | AISVA | | | AIS | | | | |
| 415 | DRB1_ | 293 | LSLQVVKMKK | 4 | 450 | VVKMKK | 0.907 | 0.694098 | 1.36 | <=WB |
| | 1301 | | AISVA | | | AIS | | | | |
| 415 | DRB1_ | 293 | LSLQVVKMKK | 4 | 450 | VVKMKK | 0.833 | 0.347651 | 3.95 | <=WB |
| | 1401 | | AISVA | | | AIS | | | | |
| 415 | DRB1_ | 293 | LSLQVVKMKK | 4 | 450 | VVKMKK | 0.833 | 0.347651 | 3.95 | <=WB |
| | 1454 | | AISVA | | | AIS | | | | |
| 416 | DRB1_ | 294 | SLQVVKMKKAI | 1 | 451 | VKMKKAI | 0.42 | 0.381103 | 2.64 | <=WB |
| | 1302 | | SVAI | | | SV | | | | |
| 416 | DRB1_ | 294 | SLQVVKMKKAI | 3 | 450 | VVKMKK | 0.487 | 0.31463 | 3.86 | <=WB |
| | 1201 | | SVAI | | | AIS | | | | |
| 417 | DRB1_ | 295 | LQVVKMKKAIS | 3 | 451 | VKMKKAI | 0.513 | 0.278718 | 4.76 | <=WB |
| | 1201 | | VAIS | | | SV | | | | |
| 417 | DRB5_ | 295 | LQVVKMKKAIS | 3 | 451 | VKMKKAI | 0.827 | 0.27436 | 3.65 | <=WB |
| | 0202 | | VAIS | | | SV | | | | |
| 418 | DRB1_ | 296 | QVVKMKKAIS | 2 | 451 | VKMKKAI | 0.873 | 0.660235 | 0.75 | <=SB |
| | 0103 | | VAISS | | | SV | | | | |
| 418 | DRB1_ | 296 | QVVKMKKAIS | 4 | 452 | MKKAISV | 0.813 | 0.479049 | 3.29 | <=WB |
| | 0102 | | VAISS | | | AI | | | | |
| 419 | DRB1_ | 297 | VVKMKKAISV | 3 | 452 | MKKAISV | 0.58 | 0.31088 | 3.62 | <=WB |
| | 0103 | | AISSV | | | AI | | | | |
| 420 | DRB1_ | 298 | VKMKKAISVAI | 2 | 452 | MKKAISV | 0.96 | 0.462854 | 2.06 | <=WB |
| | 0701 | | SSVL | | | AI | | | | |
| 420 | DRB1_ | 298 | VKMKKAISVAI | 2 | 452 | MKKAISV | 0.82 | 0.487371 | 2.41 | <=WB |
| | 0901 | | SSVL | | | AI | | | | |
| 421 | DRB1_ | 299 | KMKKAISVAIS | 5 | 453 | ISVAISSV | 0.8 | 0.362481 | 3 | <=WB |
| | 0701 | | SVLR | | | L | | | | |
| 421 | DRB1_ | 299 | KMKKAISVAIS | 5 | 453 | ISVAISSV | 0.807 | 0.5148 | 2.07 | <=WB |
| | 0901 | | SVLR | | | L | | | | |
| 422 | DRB1_ | 300 | MKKAISVAISS | 4 | 453 | ISVAISSV | 1 | 0.362291 | 3.79 | <=WB |
| | 0101 | | VLRN | | | L | | | | |
| 422 | DRB1_ | 300 | MKKAISVAISS | 4 | 453 | ISVAISSV | 1 | 0.66349 | 1.7 | <=WB |
| | 0102 | | VLRN | | | L | | | | |
| 422 | DRB1_ | 300 | MKKAISVAISS | 1 | 453 | ISVAISSV | 0.867 | 0.281264 | 4.69 | <=WB |
| | 1201 | | VLRN | | | L | | | | |
| 422 | DRB1_ | 300 | MKKAISVAISS | 4 | 453 | ISVAISSV | 0.927 | 0.377056 | 3.36 | <=WB |
| | 1401 | | VLRN | | | L | | | | |
| 422 | DRB1_ | 300 | MKKAISVAISS | 1 | 453 | ISVAISSV | 0.927 | 0.377056 | 3.36 | <=WB |
| | 1454 | | VLRN | | | L | | | | |
| 422 | DRB1_ | 300 | MKKAISVAISS | 4 | 453 | ISVAISSV | 1 | 0.505285 | 1.66 | <=WB |
| | 1501 | | VLRN | | | L | | | | |
| 422 | DRB1_ | 300 | MKKAISVAISS | 4 | 453 | ISVAISSV | 0.993 | 0.317075 | 2.23 | <=WB |
| | 1503 | | VLRN | | | L | | | | |
| 422 | DRB1_ | 300 | MKKAISVAISS | 4 | 453 | ISVAISSV | 0.993 | 0.523223 | 2.26 | <=WB |
| | 1601 | | VLRN | | | L | | | | |
| 426 | DRB1_ | 301 | ISVAISSVLRNIS | 4 | 454 | ISSVLRNI | 0.92 | 0.365367 | 3.59 | <=WB |
| | 1401 | | YV | | | S | | | | |
| 426 | DRB1_ | 301 | ISVAISSVLRNIS | 4 | 454 | ISSVLRNI | 0.92 | 0.365367 | 3.59 | <=WB |
| | 1454 | | YV | | | S | | | | |
| 427 | DRB1_ | 302 | SVAISSVLRNIS | 3 | 454 | ISSVLRNI | 1 | 0.802043 | 0.88 | <=SB |
| | 1104 | | YVN | | | S | | | | |
| 427 | DRB1_ | 302 | SVAISSVLRNIS | 3 | 454 | ISSVLRNI | 1 | 0.585763 | 2.32 | <=WB |
| | 1101 | | YVN | | | S | | | | |
| 430 | DRB1_ | 303 | ISSVLRNISYVN | 3 | 455 | VLRNISY | 0.973 | 0.314415 | 4.39 | <=WB |
| | 1302 | | KAS | | | VN | | | | |
| 430 | DRB3_ | 303 | ISSVLRNISYVN | 3 | 455 | VLRNISY | 1 | 0.330981 | 1.27 | <=WB |
| | 0202 | | KAS | | | VN | | | | |
| 433 | DRB1_ | 304 | VLRNISYVNKA | 4 | 456 | ISYVNKA | 1 | 0.906751 | 0.02 | <=SB |
| | 1401 | | SLEV | | | SL | | | | |
| 433 | DRB1_ | 304 | VLRNISYVNKA | 4 | 456 | ISYVNKA | 1 | 0.906751 | 0.02 | <=SB |
| | 1454 | | SLEV | | | SL | | | | |
| 433 | DRB1_ | 304 | VLRNISYVNKA | 1 | 456 | ISYVNKA | 0.76 | 0.437323 | 4.71 | <=WB |
| | 0801 | | SLEV | | | SL | | | | |
| 433 | DRB1_ | 304 | VLRNISYVNKA | 4 | 456 | ISYVNKA | 0.667 | 0.343053 | 3.31 | <=WB |
| | 0803 | | SLEV | | | SL | | | | |
| 434 | DRB1_ | 305 | LRNISYVNKAS | 3 | 456 | ISYVNKA | 0.767 | 0.271503 | 1.46 | <=WB |
| | 1402 | | LEVA | | | SL | | | | |
| 434 | DRB1_ | 305 | LRNISYVNKAS | 3 | 456 | ISYVNKA | 1 | 0.785303 | 0.72 | <=SB |
| | 1301 | | LEVA | | | SL | | | | |
| 434 | DRB1_ | 305 | LRNISYVNKAS | 3 | 456 | ISYVNKA | 0.84 | 0.467815 | 3.43 | <=WB |
| | 1101 | | LEVA | | | SL | | | | |
| 434 | DRB1_ | 305 | LRNISYVNKAS | 3 | 456 | ISYVNKA | 1 | 0.707032 | 1.52 | <=WB |
| | 1104 | | LEVA | | | SL | | | | |
| 434 | DRB1_ | 305 | LRNISYVNKAS | 3 | 456 | ISYVNKA | 0.987 | 0.480422 | 1.51 | <=WB |
| | 1201 | | LEVA | | | SL | | | | |
| 434 | DRB1_ | 305 | LRNISYVNKAS | 5 | 457 | YVNKASL | 0.567 | 0.586843 | 2.38 | <=WB |
| | 0801 | | LEVA | | | EV | | | | |
| 434 | DRB1_ | 305 | LRNISYVNKAS | 5 | 457 | YVNKASL | 0.573 | 0.463048 | 1.44 | <=WB |
| | 0803 | | LEVA | | | EV | | | | |
| 435 | DRB1_ | 306 | RNISYVNKASL | 2 | 456 | ISYVNKA | 0.513 | 0.51778 | 0.87 | <=SB |
| | 1302 | | EVAS | | | SL | | | | |
| 435 | DRB1_ | 306 | RNISYVNKASL | 4 | 457 | YVNKASL | 0.887 | 0.367383 | 2.94 | <=WB |
| | 0701 | | EVAS | | | EV | | | | |
| 435 | DRB1_ | 306 | RNISYVNKASL | 4 | 457 | YVNKASL | 0.52 | 0.386634 | 4.39 | <=WB |
| | 1101 | | EVAS | | | EV | | | | |
| 435 | DRB1_ | 306 | RNISYVNKASL | 4 | 457 | YVNKASL | 0.953 | 0.553274 | 1.97 | <=WB |
| | 1601 | | EVAS | | | EV | | | | |
| 436 | DRB1_ | 307 | NISYVNKASLE | 3 | 457 | YVNKASL | 0.86 | 0.415334 | 2.01 | <=WB |
| | 1302 | | VASS | | | EV | | | | |
| 436 | DRB3_ | 307 | NISYVNKASLE | 3 | 457 | YVNKASL | 0.867 | 0.131611 | 3.62 | <=WB |
| | 0202 | | VASS | | | EV | | | | |
| 436 | DRB5_ | 307 | NISYVNKASLE | 3 | 457 | YVNKASL | 0.947 | 0.332275 | 3.91 | <=WB |
| | 0101 | | VASS | | | EV | | | | |
| 436 | DRB1_ | 307 | NISYVNKASLE | 3 | 457 | YVNKASL | 1 | 0.85132 | 0.19 | <=SB |
| | 0103 | | VASS | | | EV | | | | |
| 436 | DRB1_ | 307 | NISYVNKASLE | 3 | 457 | YVNKASL | 1 | 0.323804 | 4.21 | <=WB |
| | 0101 | | VASS | | | EV | | | | |
| 442 | DRB1_ | 308 | KASLEVASSSQ | 3 | 458 | LEVASSS | 1 | 0.428372 | 4.11 | <=WB |
| | 1001 | | NLSS | | | QN | | | | |
| 462 | DRB1_ | 309 | ASALEEMSANV | 3 | 459 | LEEMSAN | 0.993 | 0.424955 | 4.05 | <=WB |
| | 0403 | | EQIA | | | VE | | | | |
| 462 | DRB1_ | 309 | ASALEEMSANV | 3 | 459 | LEEMSAN | 1 | 0.374162 | 2.95 | <=WB |
| | 0405 | | EQIA | | | VE | | | | |
| 472 | DRB1_ | 310 | VEQIASGVNMS | 3 | 460 | IASGVNM | 0.987 | 0.268505 | 4.62 | <=WB |
| | 0402 | | ANNS | | | SA | | | | |
| 472 | DRB1_ | 310 | VEQIASGVNMS | 3 | 460 | IASGVNM | 1 | 0.420091 | 3.59 | <=WB |
| | 0404 | | ANNS | | | SA | | | | |
| 476 | DRB1_ | 311 | ASGVNMSANN | 3 | 461 | VNMSAN | 0.84 | 0.451631 | 1.55 | <=WB |
| | 1302 | | SYETE | | | NSY | | | | |
| 476 | DRB1_ | 311 | ASGVNMSANN | 3 | 461 | VNMSAN | 0.993 | 0.298286 | 4.25 | <=WB |
| | 1201 | | SYETE | | | NSY | | | | |
| 476 | DRB1_ | 311 | ASGVNMSANN | 3 | 461 | VNMSAN | 0.973 | 0.391459 | 3.09 | <=WB |
| | 1401 | | SYETE | | | NSY | | | | |
| 476 | DRB1_ | 311 | ASGVNMSANN | 3 | 461 | VNMSAN | 0.973 | 0.391459 | 3.09 | <=WB |
| | 1454 | | SYETE | | | NSY | | | | |
| 477 | DRB3_ | 312 | SGVNMSANNS | 4 | 462 | MSANNSY | 0.92 | 0.225131 | 2.03 | <=WB |
| | 0202 | | YETEQ | | | ET | | | | |
| 484 | DRB5_ | 313 | NNSYETEQIAL | 3 | 463 | YETEQIA | 1 | 0.57315 | 1.37 | <=WB |
| | 0101 | | KTNE | | | LK | | | | |
| 489 | DRB1_ | 314 | TEQIALKTNEN | 3 | 464 | IALKTNE | 1 | 0.874408 | 0.11 | <=SB |
| | 0402 | | SQIG | | | NS | | | | |
| 489 | DRB1_ | 314 | TEQIALKTNEN | 3 | 464 | IALKTNE | 0.967 | 0.42248 | 3.56 | <=WB |
| | 0404 | | SQIG | | | NS | | | | |
| 489 | DRB1_ | 314 | TEQIALKTNEN | 3 | 464 | IALKTNE | 0.987 | 0.440285 | 4.22 | <=WB |
| | 1104 | | SQIG | | | NS | | | | |
| 489 | DRB4_ | 314 | TEQIALKTNEN | 3 | 464 | IALKTNE | 0.927 | 0.182885 | 2.59 | <=WB |
| | 0101 | | SQIG | | | NS | | | | |
| 489 | DRB4_ | 314 | TEQIALKTNEN | 3 | 464 | IALKTNE | 0.927 | 0.182885 | 2.59 | <=WB |
| | 0103 | | SQIG | | | NS | | | | |
| 489 | DRB5_ | 314 | TEQIALKTNEN | 3 | 464 | TALKTNE | 0.893 | 0.243033 | 4.33 | <=WB |
| | 0202 | | SQIG | | | NS | | | | |
| 491 | DRB1_ | 315 | QIALKTNENSQI | 3 | 465 | LKTNENS | 1 | 0.607383 | 0.36 | <=SB |
| | 1302 | | GGR | | | QI | | | | |
| 491 | DRB1_ | 315 | QIALKTNENSQI | 3 | 465 | LKTNENS | 0.987 | 0.241298 | 1.96 | <=WB |
| | 1402 | | GGR | | | QI | | | | |
| 491 | DRB3_ | 315 | QIALKTNENSQI | 3 | 465 | LKTNENS | 1 | 0.726606 | 0.24 | <=SB |
| | 0202 | | GGR | | | QI | | | | |
| 491 | DRB1_ | 315 | QIALKTNENSQI | 3 | 465 | LKTNENS | 0.993 | 0.147688 | 3.37 | <=WB |
| | 0305 | | GGR | | | QI | | | | |
| 491 | DRB1_ | 315 | QIALKTNENSQI | 3 | 465 | LKTNENS | 0.987 | 0.403339 | 2.9 | <=WB |
| | 1401 | | GGR | | | QI | | | | |
| 491 | DRB1_ | 315 | QIALKTNENSQI | 3 | 465 | LKTNENS | 0.987 | 0.403339 | 2.9 | <=WB |
| | 1454 | | GGR | | | QI | | | | |
| 491 | DRB3_ | 315 | QIALKTNENSQI | 3 | 465 | LKTNENS | 1 | 0.156187 | 3.52 | <=WB |
| | 0101 | | GGR | | | QI | | | | |
| 507 | DRB4_ | 316 | VEESVIAMQDI | 4 | 466 | VIAMQDI | 0.553 | 0.19186 | 2.41 | <=WB |
| | 0101 | | VEKV | | | VE | | | | |
| 508 | DRB1_ | 317 | EESVIAMQDIV | 3 | 466 | VIAMQDI | 0.953 | 0.369893 | 3.41 | <=WB |
| | 0401 | | EKVS | | | VE | | | | |
| 508 | DRB1_ | 317 | EESVIAMQDIV | 3 | 466 | VIAMQDI | 0.993 | 0.692422 | 1.03 | <=WB |
| | 0408 | | EKVS | | | VE | | | | |
| 508 | DRB1_ | 317 | EESVIAMQDIV | 3 | 466 | VIAMQDI | 1 | 0.954088 | 0.01 | <=SB |
| | 0403 | | EKVS | | | VE | | | | |
| 508 | DRB1_ | 317 | EESVIAMQDIV | 3 | 466 | VIAMQDI | 1 | 0.918624 | 0.17 | <=SB |
| | 0404 | | EKVS | | | VE | | | | |
| 508 | DRB1_ | 317 | EESVIAMQDIV | 3 | 466 | VIAMQDI | 1 | 0.933505 | 0.11 | <=SB |
| | 0405 | | EKVS | | | VE | | | | |
| 508 | DRB4_ | 317 | EESVIAMQDIV | 4 | 467 | IAMQDIV | 0.52 | 0.261239 | 1.43 | <=WB |
| | 0101 | | EKVS | | | EK | | | | |
| 508 | DRB4_ | 317 | EESVIAMQDIV | 4 | 467 | IAMQDIV | 0.52 | 0.261239 | 1.43 | <=WB |
| | 0103 | | EKVS | | | EK | | | | |
| 508 | DRB1_ | 317 | EESVIAMQDIV | 3 | 466 | VIAMQDI | 0.953 | 0.519087 | 1.42 | <=WB |
| | 0402 | | EKVS | | | VE | | | | |
| 508 | DRB1_ | 317 | EESVIAMQDIV | 3 | 466 | VIAMQDI | 0.92 | 0.295243 | 4.63 | <=WB |
| | 0803 | | EKVS | | | VE | | | | |
| 513 | DRB1_ | 318 | AMQDIVEKVSV | 4 | 468 | IVEKVSVI | 0.933 | 0.45457 | 4.36 | <=WB |
| | 0801 | | IEEI | | | E | | | | |
| 514 | DRB1_ | 319 | MQDIVEKVSVI | 3 | 468 | IVEKVSVI | 0.873 | 0.414887 | 2.02 | <=WB |
| | 0803 | | EEIA | | | E | | | | |
| 515 | DRB1_ | 320 | QDIVEKVSVIEE | 3 | 469 | VEKVSVI | 0.98 | 0.52015 | 2.69 | <=WB |
| | 0403 | | IAR | | | EE | | | | |
| 515 | DRB1_ | 320 | QDIVEKVSVIEE | 3 | 469 | VEKVSVI | 0.973 | 0.348069 | 3.27 | <=WB |
| | 0405 | | IAR | | | EE | | | | |
| 518 | DRB5_ | 321 | VEKVSVIEEIAR | 3 | 470 | VSVIEEIA | 0.88 | 0.51502 | 1.77 | <=WB |
| | 0101 | | KTN | | | R | | | | |
| 518 | DRB5_ | 321 | VEKVSVIEEIAR | 3 | 470 | VSVIEEIA | 0.947 | 0.597091 | 0.72 | <=SB |
| | 0202 | | KTN | | | R | | | | |
| 518 | DRB1_ | 321 | VEKVSVIEEIAR | 3 | 470 | VSVIEEIA | 0.96 | 0.229351 | 4.88 | <=WB |
| | 0301 | | KTN | | | R | | | | |
| 518 | DRB1_ | 321 | VEKVSVIEEIAR | 3 | 470 | VSVIEEIA | 0.847 | 0.374424 | 2.75 | <=WB |
| | 1201 | | KTN | | | R | | | | |
| 518 | DRB1_ | 321 | VEKVSVIEEIAR | 3 | 470 | VSVIEEIA | 0.933 | 0.629963 | 2.01 | <=WB |
| | 1301 | | KTN | | | R | | | | |
| 519 | DRB1_ | 322 | EKVSVIEEIARK | 4 | 471 | VIEEIARK | 0.973 | 0.471433 | 3.37 | <=WB |
| | 0102 | | TNL | | | T | | | | |
| 519 | DRB1_ | 322 | EKVSVIEEIARK | 4 | 471 | VIEEIARK | 0.993 | 0.335953 | 3.24 | <=WB |
| | 0103 | | TNL | | | T | | | | |
| 521 | DRB1_ | 323 | VSVIEEIARKTN | 3 | 472 | IEEIARKT | 0.993 | 0.357329 | 4.83 | <=WB |
| | 1101 | | LLA | | | N | | | | |
| 522 | DRB1_ | 324 | SVIEEIARKTNL | 2 | 472 | IEEIARKT | 0.533 | 0.391432 | 4.98 | <=WB |
| | 1104 | | LAL | | | N | | | | |
| 523 | DRB1_ | 325 | VIEEIARKTNLL | 4 | 473 | IARKTNL | 0.967 | 0.433016 | 4.32 | <=WB |
| | 1104 | | ALN | | | LA | | | | |
| 524 | DRB1_ | 326 | IEEIARKTNLLA | 3 | 473 | IARKTNL | 0.953 | 0.306158 | 4.67 | <=WB |
| | 1302 | | LNA | | | LA | | | | |
| 524 | DRB1_ | 326 | IEEIARKTNLLA | 3 | 473 | IARKTNL | 1 | 0.840895 | 0.17 | <=SB |
| | 0402 | | LNA | | | LA | | | | |
| 524 | DRB1_ | 326 | IEEIARKTNLLA | 3 | 473 | IARKTNL | 0.98 | 0.277509 | 4.18 | <=WB |
| | 0103 | | LNA | | | LA | | | | |
| 524 | DRB1_ | 326 | IEEIARKTNLLA | 3 | 473 | IARKTNL | 1 | 0.343818 | 4.69 | <=WB |
| | 0404 | | LNA | | | LA | | | | |
| 524 | DRB1_ | 326 | IEEIARKTNLLA | 3 | 473 | IARKTNL | 1 | 0.588103 | 2.46 | <=WB |
| | 1301 | | LNA | | | LA | | | | |
| 524 | DRB1_ | 326 | IEEIARKTNLLA | 3 | 473 | IARKTNL | 1 | 0.261372 | 3.81 | <=WB |
| | 1501 | | LNA | | | LA | | | | |
| 524 | DRB1_ | 326 | IEEIARKTNLLA | 3 | 473 | IARKTNL | 0.993 | 0.17812 | 4.73 | <=WB |
| | 1503 | | LNA | | | LA | | | | |
| 524 | DRB5_ | 326 | IEEIARKTNLLA | 3 | 473 | IARKTNL | 0.953 | 0.262545 | 3.9 | <=WB |
| | 0202 | | LNA | | | LA | | | | |
| 529 | DRB1_ | 327 | RKTNLLALNAA | 4 | 474 | LLALNAA | 0.987 | 0.395892 | 2.73 | <=WB |
| | 0405 | | IEAA | | | IE | | | | |
| 530 | DRB3_ | 328 | KTNLLALNAAI | 4 | 475 | LALNAAI | 1 | 0.765284 | 0.19 | <=SB |
| | 0202 | | EAAR | | | EA | | | | |
| 530 | DRB1_ | 328 | KTNLLALNAAI | 3 | 474 | LLALNAA | 0.833 | 0.407527 | 3.58 | <=WB |
| | 0408 | | EAAR | | | IE | | | | |
| 530 | DRB1_ | 328 | KTNLLALNAAI | 3 | 474 | LLALNAA | 0.927 | 0.747298 | 0.8 | <=SB |
| | 1001 | | EAAR | | | IE | | | | |
| 530 | DRB1_ | 328 | KTNLLALNAAI | 3 | 474 | LLALNAA | 0.893 | 0.501192 | 2.51 | <=WB |
| | 0101 | | EAAR | | | IE | | | | |
| 530 | DRB1_ | 328 | KTNLLALNAAI | 3 | 474 | LLALNAA | 0.84 | 0.70659 | 1.43 | <=WB |
| | 0102 | | EAAR | | | IE | | | | |
| 530 | DRB1_ | 328 | KTNLLALNAAI | 3 | 474 | LLALNAA | 0.653 | 0.316491 | 3.53 | <=WB |
| | 0103 | | EAAR | | | IE | | | | |
| 530 | DRB1_ | 328 | KTNLLALNAAI | 3 | 474 | LLALNAA | 0.94 | 0.536182 | 2.48 | <=WB |
| | 0403 | | EAAR | | | IE | | | | |
| 530 | DRB1_ | 328 | KTNLLALNAAI | 3 | 474 | LLALNAA | 0.833 | 0.426917 | 3.5 | <=WB |
| | 0404 | | EAAR | | | IE | | | | |
| 530 | DRB1_ | 328 | KTNLLALNAAI | 3 | 474 | LLALNAA | 0.973 | 0.545325 | 2.89 | <=WB |
| | 0801 | | EAAR | | | IE | | | | |
| 530 | DRB1_ | 328 | KTNLLALNAAI | 3 | 474 | LLALNAA | 0.86 | 0.364965 | 2.85 | <=WB |
| | 0803 | | EAAR | | | IE | | | | |
| 530 | DRB1_ | 328 | KTNLLALNAAI | 3 | 474 | LLALNAA | 0.933 | 0.473984 | 1.56 | <=WB |
| | 1201 | | EAAR | | | IE | | | | |
| 531 | DRB1_ | 329 | TNLLALNAAIE | 3 | 475 | LALNAAI | 1 | 0.558755 | 0.59 | <=SB |
| | 1302 | | AARA | | | EA | | | | |
| 531 | DRB1_ | 329 | TNLLALNAAIE | 3 | 475 | LALNAAI | 0.933 | 0.319425 | 0.92 | <=SB |
| | 1402 | | AARA | | | EA | | | | |
| 531 | DRB1_ | 329 | TNLLALNAAIE | 3 | 475 | LALNAAI | 0.66 | 0.284436 | 4.72 | <=WB |
| | 0101 | | AARA | | | EA | | | | |
| 531 | DRB1_ | 329 | TNLLALNAAIE | 3 | 475 | LALNAAI | 0.653 | 0.504676 | 3.02 | <=WB |
| | 0102 | | AARA | | | EA | | | | |
| 531 | DRB1_ | 329 | TNLLALNAAIE | 3 | 475 | LALNAAI | 0.78 | 0.240913 | 4.93 | <=WB |
| | 0103 | | AARA | | | EA | | | | |
| 533 | DRB5_ | 330 | LLALNAAIEAA | 3 | 476 | LNAAIEA | 0.96 | 0.300805 | 4.57 | <=WB |
| | 0101 | | RAGD | | | AR | | | | |
| 548 | DRB1_ | 331 | EGKGFAVVASE | 4 | 477 | FAVVASE | 0.873 | 0.322216 | 4.16 | <=WB |
| | 1302 | | IRKL | | | IR | | | | |
| 549 | DRB1_ | 332 | GKGFAVVASEI | 3 | 477 | FAVVASE | 0.9 | 0.294337 | 1.18 | <=WB |
| | 1402 | | RKLA | | | IR | | | | |
| 549 | DRB3_ | 332 | GKGFAVVASEI | 3 | 477 | FAVVASE | 0.907 | 0.118989 | 4.01 | <=WB |
| | 0202 | | RKLA | | | IR | | | | |
| 549 | DRB5_ | 332 | GKGFAVVASEI | 3 | 477 | FAVVASE | 1 | 0.930899 | 0.03 | <=SB |
| | 0101 | | RKLA | | | IR | | | | |
| 549 | DRB1_ | 332 | GKGFAVVASEI | 3 | 477 | FAVVASE | 1 | 0.865651 | 0.23 | <=SB |
| | 1601 | | RKLA | | | IR | | | | |
| 549 | DRB1_ | 332 | GKGFAVVASEI | 3 | 477 | FAVVASE | 0.98 | 0.275603 | 4.86 | <=WB |
| | 0101 | | RKLA | | | IR | | | | |
| 549 | DRB1_ | 332 | GKGFAVVASEI | 3 | 477 | FAVVASE | 0.887 | 0.250239 | 4.72 | <=WB |
| | 0103 | | RKLA | | | IR | | | | |
| 549 | DRB1_ | 332 | GKGFAVVASEI | 3 | 477 | FAVVASE | 0.927 | 0.136327 | 3.64 | <=WB |
| | 0305 | | RKLA | | | IR | | | | |
| 549 | DRB1_ | 332 | GKGFAVVASEI | 3 | 477 | FAVVASE | 0.76 | 0.358165 | 3.05 | <=WB |
| | 0701 | | RKLA | | | IR | | | | |
| 549 | DRB1_ | 332 | GKGFAVVASEI | 3 | 477 | FAVVASE | 0.887 | 0.296598 | 4.58 | <=WB |
| | 0803 | | RKLA | | | IR | | | | |
| 549 | DRB1_ | 332 | GKGFAVVASEI | 3 | 477 | FAVVASE | 0.673 | 0.51736 | 2.04 | <=WB |
| | 0901 | | RKLA | | | IR | | | | |
| 549 | DRB1_ | 332 | GKGFAVVASEI | 3 | 477 | FAVVASE | 0.653 | 0.349331 | 3.92 | <=WB |
| | 1401 | | RKLA | | | IR | | | | |
| 549 | DRB1_ | 332 | GKGFAVVASEI | 3 | 477 | FAVVASE | 0.653 | 0.349331 | 3.92 | <=WB |
| | 1454 | | RKLA | | | IR | | | | |
| 549 | DRB1_ | 332 | GKGFAVVASEI | 3 | 477 | FAVVASE | 0.887 | 0.169603 | 4.99 | <=WB |
| | 1503 | | RKLA | | | IR | | | | |
| 549 | DRB3_ | 332 | GKGFAVVASEI | 3 | 477 | FAVVASE | 0.927 | 0.119122 | 4.34 | <=WB |
| | 0101 | | RKLA | | | IR | | | | |
| 549 | DRB5_ | 332 | GKGFAVVASEI | 3 | 477 | FAVVASE | 0.947 | 0.466 | 1.42 | <=WB |
| | 0202 | | RKLA | | | IR | | | | |
| 550 | DRB1_ | 333 | KGFAVVASEIR | 1 | 478 | VVASEIR | 0.66 | 0.31461 | 3.66 | <=WB |
| | 0701 | | KLAD | | | KL | | | | |
| 550 | DRB1_ | 333 | KGFAVVASEIR | 4 | 478 | VVASEIR | 0.693 | 0.485536 | 2.43 | <=WB |
| | 0901 | | KLAD | | | KL | | | | |
| 550 | DRB1_ | 333 | KGFAVVASEIR | 4 | 478 | VVASEIR | 0.513 | 0.342191 | 4.08 | <=WB |
| | 1401 | | KLAD | | | KL | | | | |
| 550 | DRB1_ | 333 | KGFAVVASEIR | 4 | 478 | VVASEIR | 0.513 | 0.342191 | 4.08 | <=WB |
| | 1454 | | KLAD | | | KL | | | | |
| 552 | DRB1_ | 334 | FAVVASEIRKL | 3 | 479 | VASEIRK | 1 | 0.595807 | 2.41 | <=WB |
| | 1104 | | ADLS | | | LA | | | | |
| 555 | DRB5_ | 335 | VASEIRKLADL | 4 | 480 | IRKLADL | 0.993 | 0.43378 | 2.48 | <=WB |
| | 0101 | | SKIS | | | SK | | | | |
| 556 | DRB1_ | 336 | ASEIRKLADLS | 3 | 480 | IRKLADL | 0.993 | 0.297977 | 3.97 | <=WB |
| | 0402 | | KISA | | | SK | | | | |
| 556 | DRB5_ | 336 | ASEIRKLADLS | 3 | 480 | IRKLADL | 0.993 | 0.477201 | 1.34 | <=WB |
| | 0202 | | KISA | | | SK | | | | |
| 558 | DRB1_ | 337 | EIRKLADLSKIS | 4 | 481 | LADLSKIS | 1 | 0.6196 | 2.05 | <=WB |
| | 1101 | | ALE | | | A | | | | |
| 558 | DRB1_ | 337 | EIRKLADLSKIS | 4 | 481 | LADLSKIS | 1 | 0.776212 | 1.04 | <=WB |
| | 1104 | | ALE | | | A | | | | |
| 561 | DRB1_ | 338 | KLADLSKISAL | 4 | 482 | LSKISALE | 1 | 0.320417 | 4.25 | <=WB |
| | 0101 | | EIGE | | | I | | | | |
| 561 | DRB1_ | 338 | KLADLSKISAL | 4 | 482 | LSKISALE | 1 | 0.557021 | 1.41 | <=WB |
| | 0701 | | EIGE | | | I | | | | |
| 561 | DRB1_ | 338 | KLADLSKISAL | 4 | 482 | LSKISALE | 0.993 | 0.364834 | 4.65 | <=WB |
| | 0901 | | EIGE | | | I | | | | |
| 561 | DRB1_ | 338 | KLADLSKISAL | 4 | 482 | LSKISALE | 0.993 | 0.439344 | 1.89 | <=WB |
| | 1201 | | EIGE | | | I | | | | |
| 562 | DRB1_ | 339 | LADLSKISALEI | 3 | 482 | LSKISALE | 1 | 0.572221 | 2.39 | <=WB |
| | 0102 | | GEL | | | I | | | | |
| 565 | DRB1_ | 340 | LSKISALEIGEL | 3 | 483 | ISALEIGE | 0.987 | 0.309139 | 3.98 | <=WB |
| | 1201 | | VED | | | L | | | | |
| 570 | DRB4_ | 341 | ALEIGELVEDN | 3 | 484 | IGELVED | 0.94 | 0.169477 | 2.88 | <=WB |
| | 0101 | | SKVA | | | NS | | | | |
| 570 | DRB4_ | 341 | ALEIGELVEDN | 3 | 484 | IGELVED | 0.94 | 0.169477 | 2.88 | <=WB |
| | 0103 | | SKVA | | | NS | | | | |
| 573 | DRB1_ | 342 | IGELVEDNSKV | 3 | 485 | LVEDNSK | 1 | 0.624124 | 1.22 | <=WB |
| | 0401 | | ATEA | | | VA | | | | |
| 573 | DRB1_ | 342 | IGELVEDNSKV | 3 | 485 | LVEDNSK | 1 | 0.37978 | 3.97 | <=WB |
| | 0408 | | ATEA | | | VA | | | | |
| 573 | DRB1_ | 342 | IGELVEDNSKV | 3 | 485 | LVEDNSK | 0.687 | 0.469235 | 1.34 | <=WB |
| | 1302 | | ATEA | | | VA | | | | |
| 573 | DRB1_ | 342 | IGELVEDNSKV | 3 | 485 | LVEDNSK | 0.82 | 0.310291 | 1.01 | <=WB |
| | 1402 | | ATEA | | | VA | | | | |
| 573 | DRB3_ | 342 | IGELVEDNSKV | 3 | 485 | LVEDNSK | 0.88 | 0.243731 | 1.86 | <=WB |
| | 0202 | | ATEA | | | VA | | | | |
| 573 | DRB3_ | 342 | IGELVEDNSKV | 3 | 485 | LVEDNSK | 1 | 0.49968 | 0.91 | <=SB |
| | 0101 | | ATEA | | | VA | | | | |
| 573 | DRB1_ | 342 | IGELVEDNSKV | 3 | 485 | LVEDNSK | 0.967 | 0.642027 | 1.25 | <=WB |
| | 0301 | | ATEA | | | VA | | | | |
| 573 | DRB1_ | 342 | IGELVEDNSKV | 3 | 485 | LVEDNSK | 0.953 | 0.348796 | 1.15 | <=WB |
| | 0305 | | ATEA | | | VA | | | | |
| 573 | DRB1_ | 342 | IGELVEDNSKV | 3 | 485 | LVEDNSK | 1 | 0.42839 | 3.48 | <=WB |
| | 0404 | | ATEA | | | VA | | | | |
| 574 | DRB1_ | 343 | GELVEDNSKVA | 3 | 486 | VEDNSKV | 0.54 | 0.396997 | 2.34 | <=WB |
| | 1302 | | TEAG | | | AT | | | | |
| 579 | DRB1_ | 344 | DNSKVATEAG | 3 | 487 | KVATEAG | 0.827 | 0.253557 | 4.69 | <=WB |
| | 0701 | | VIFKE | | | VI | | | | |
| 579 | DRB1_ | 344 | DNSKVATEAG | 4 | 488 | VATEAGV | 0.98 | 0.306856 | 4.04 | <=WB |
| | 1201 | | VIFKE | | | IF | | | | |
| 586 | DRB1_ | 345 | EAGVIFKEMLP | 3 | 489 | VIFKEML | 0.713 | 0.454922 | 4.35 | <=WB |
| | 0801 | | EIEE | | | PE | | | | |
| 586 | DRB1_ | 345 | EAGVIFKEMLP | 3 | 489 | VIFKEML | 0.72 | 0.286124 | 4.93 | <=WB |
| | 0803 | | EIEE | | | PE | | | | |
| 587 | DRB1_ | 346 | AGVIFKEMLPEI | 4 | 490 | FKEMLPEI | 1 | 0.371581 | 2.98 | <=WB |
| | 0405 | | EET | | | E | | | | |
| 588 | DRB1_ | 347 | GVIFKEMLPEIE | 3 | 490 | FKEMLPEI | 1 | 0.745215 | 0.82 | <=SB |
| | 1001 | | ETA | | | E | | | | |
| 595 | DRB5_ | 348 | LPEIEETANLVK | 3 | 491 | IEETANL | 1 | 0.406098 | 2.83 | <=WB |
| | 0101 | | KIS | | | VK | | | | |
| 595 | DRB5_ | 348 | LPEIEETANLVK | 3 | 491 | IEETANL | 1 | 0.279868 | 3.54 | <=WB |
| | 0202 | | KIS | | | VK | | | | |
| 601 | DRB1_ | 349 | TANLVKKISEG | 3 | 492 | LVKKISE | 1 | 0.398584 | 2.47 | <=WB |
| | 0103 | | SSKQ | | | GS | | | | |
| 601 | DRB1_ | 349 | TANLVKKISEG | 3 | 492 | LVKKISE | 0.987 | 0.530768 | 1.35 | <=WB |
| | 0402 | | SSKQ | | | GS | | | | |
| 601 | DRB1_ | 349 | TANLVKKISEG | 3 | 492 | LVKKISE | 0.707 | 0.403941 | 4.77 | <=WB |
| | 1104 | | SSKQ | | | GS | | | | |
| 616 | DRB1_ | 350 | SDQIAQFKMAL | 3 | 493 | IAQFKMA | 0.827 | 0.286333 | 4.92 | <=WB |
| | 0803 | | DQVG | | | LD | | | | |
| 618 | DRB1_ | 351 | QIAQFKMALDQ | 4 | 494 | FKMALD | 1 | 0.809797 | 0.41 | <=SB |
| | 0401 | | VGEV | | | QVG | | | | |
| 618 | DRB1_ | 351 | QIAQFKMALDQ | 4 | 494 | FKMALD | 1 | 0.725913 | 0.9 | <=SB |
| | 0403 | | VGEV | | | QVG | | | | |
| 618 | DRB1_ | 351 | QIAQFKMALDQ | 4 | 494 | FKMALD | 0.987 | 0.407551 | 2.4 | <=WB |
| | 0402 | | VGEV | | | QVG | | | | |
| 619 | DRB1_ | 352 | IAQFKMALDQV | 3 | 494 | FKMALD | 1 | 0.868822 | 0.24 | <=SB |
| | 0408 | | GEVV | | | QVG | | | | |
| 619 | DRB1_ | 352 | IAQFKMALDQV | 3 | 494 | FKMALD | 1 | 0.594048 | 1.89 | <=WB |
| | 0404 | | GEVV | | | QVG | | | | |
| 619 | DRB1_ | 352 | IAQFKMALDQV | 3 | 494 | FKMALD | 1 | 0.561951 | 1.46 | <=WB |
| | 0405 | | GEVV | | | QVG | | | | |
| 619 | DRB1_ | 352 | IAQFKMALDQV | 3 | 494 | FKMALD | 0.953 | 0.367816 | 4.57 | <=WB |
| | 0901 | | GEVV | | | QVG | | | | |
| 619 | DRB1_ | 352 | IAQFKMALDQV | 3 | 494 | FKMALD | 1 | 0.59388 | 1.98 | <=WB |
| | 1001 | | GEVV | | | QVG | | | | |
| 619 | DRB1_ | 352 | IAQFKMALDQV | 3 | 494 | FKMALD | 1 | 0.327761 | 4.88 | <=WB |
| | 1601 | | GEVV | | | QVG | | | | |
| 621 | DRB1_ | 353 | QFKMALDQVG | 3 | 495 | MALDQV | 1 | 0.384996 | 2.93 | <=WB |
| | 0301 | | EVVQS | | | GEV | | | | |
| 621 | DRB1_ | 353 | QFKMALDQVG | 3 | 495 | MALDQV | 0.987 | 0.153241 | 3.24 | <=WB |
| | 0305 | | EVVQS | | | GEV | | | | |
| 621 | DRB3_ | 353 | QFKMALDQVG | 3 | 495 | MALDQV | 1 | 0.304726 | 1.88 | <=WB |
| | 0101 | | EVVQS | | | GEV | | | | |
| 640 | DRB1_ | 354 | SEQLSSMSDKM | 3 | 496 | LSSMSDK | 1 | 0.377962 | 4.48 | <=WB |
| | 0102 | | LEKS | | | ML | | | | |
| 640 | DRB1_ | 354 | SEQLSSMSDKM | 3 | 496 | LSSMSDK | 1 | 0.297222 | 3.34 | <=WB |
| | 1501 | | LEKS | | | ML | | | | |
| 640 | DRB1_ | 354 | SEQLSSMSDKM | 3 | 496 | LSSMSDK | 0.98 | 0.228348 | 3.49 | <=WB |
| | 1503 | | LEKS | | | ML | | | | |
| 640 | DRB1_ | 354 | SEQLSSMSDKM | 3 | 496 | LSSMSDK | 1 | 0.342421 | 4.6 | <=WB |
| | 1601 | | LEKS | | | ML | | | | |
| 647 | DRB1_ | 355 | SDKMLEKSKEL | 3 | 497 | MLEKSKE | 0.947 | 0.790161 | 0.69 | <=SB |
| | 1301 | | RKSV | | | LR | | | | |
| 658 | DRB1_ | 356 | RKSVLFFKIKDS | 3 | 498 | VLFFKIK | 1 | 0.537898 | 1.3 | <=WB |
| | 0402 | | KIE | | | DS | | | | |
| 658 | DRB1_ | 356 | RKSVLFFKIKDS | 3 | 498 | VLFFKIK | 0.807 | 0.206174 | 4.85 | <=WB |
| | 1501 | | KIE | | | DS | | | | |
| 658 | DRB1_ | 356 | RKSVLFFKIKDS | 3 | 498 | VLFFKIK | 0.807 | 0.24134 | 3.26 | <=WB |
| | 1503 | | KIE | | | DS | | | | |
| 658 | DRB5_ | 356 | RKSVLFFKIKDS | 3 | 498 | VLFFKIK | 0.807 | 0.249716 | 4.17 | <=WB |
| | 0202 | | KIE | | | DS | | | | |
| 659 | DRB1_ | 357 | KSVLFFKIKDS | 1 | 499 | FFKIKDSK | 0.507 | 0.181313 | 4.63 | <=WB |
| | 1503 | | KIEN | | | I | | | | |
| 659 | DRB1_ | 357 | KSVLFFKIKDS | 3 | 500 | LFFKIKDS | 0.787 | 0.438346 | 4.77 | <=WB |
| | 1301 | | KIEN | | | K | | | | |
| 660 | DRB1_ | 358 | SVLFFKIKDSKI | 3 | 499 | FFKIKDSK | 0.947 | 0.333311 | 3.96 | <=WB |
| | 0401 | | ENP | | | I | | | | |
| 660 | DRB1_ | 358 | SVLFFKIKDSKI | 3 | 499 | FFKIKDSK | 0.973 | 0.460272 | 2.97 | <=WB |
| | 0408 | | ENP | | | I | | | | |
| 660 | DRB3_ | 358 | SVLFFKIKDSKI | 3 | 499 | FFKIKDSK | 0.747 | 0.10369 | 4.61 | <=WB |
| | 0202 | | ENP | | | I | | | | |
| 660 | DRB5_ | 358 | SVLFFKIKDSKI | 3 | 499 | FFKIKDSK | 0.773 | 0.430829 | 2.52 | <=WB |
| | 0101 | | ENP | | | I | | | | |
| 660 | DRB1_ | 358 | SVLFFKIKDSKI | 3 | 499 | FFKIKDSK | 0.967 | 0.722853 | 0.8 | <=SB |
| | 1601 | | ENP | | | I | | | | |
| 660 | DRB1_ | 358 | SVLFFKIKDSKI | 3 | 499 | FFKIKDSK | 0.993 | 0.376092 | 3.64 | <=WB |
| | 0101 | | ENP | | | I | | | | |
| 660 | DRB1_ | 358 | SVLFFKIKDSKI | 3 | 499 | FFKIKDSK | 0.72 | 0.415348 | 2.32 | <=WB |
| | 0103 | | ENP | | | I | | | | |
| 660 | DRB1_ | 358 | SVLFFKIKDSKI | 3 | 499 | FFKIKDSK | 0.833 | 0.452006 | 2.22 | <=WB |
| | 0405 | | ENP | | | I | | | | |
| 660 | DRB1_ | 358 | SVLFFKIKDSKI | 3 | 499 | FFKIKDSK | 0.973 | 0.575531 | 1.32 | <=WB |
| | 0701 | | ENP | | | I | | | | |
| 660 | DRB1_ | 358 | SVLFFKIKDSKI | 3 | 499 | FFKIKDSK | 0.927 | 0.3781 | 4.33 | <=WB |
| | 0901 | | ENP | | | I | | | | |
| 660 | DRB1_ | 358 | SVLFFKIKDSKI | 3 | 499 | FFKIKDSK | 0.913 | 0.228225 | 4.4 | <=WB |
| | 1501 | | ENP | | | I | | | | |
| 661 | DRB1_ | 359 | VLFFKIKDSKIE | 3 | 501 | FKIKDSKI | 0.973 | 0.508511 | 2.32 | <=WB |
| | 1303 | | NPE | | | E | | | | |
| 661 | DRB5_ | 359 | VLFFKIKDSKIE | 3 | 501 | FKIKDSKI | 0.58 | 0.31743 | 4.21 | <=WB |
| | 0101 | | NPE | | | E | | | | |
| 661 | DRB1_ | 359 | VLFFKIKDSKIE | 3 | 501 | FKIKDSKI | 1 | 0.85329 | 0.36 | <=SB |
| | 0801 | | NPE | | | E | | | | |
| 661 | DRB1_ | 359 | VLFFKIKDSKIE | 3 | 501 | FKIKDSKI | 0.967 | 0.672206 | 0.27 | <=SB |
| | 0803 | | NPE | | | E | | | | |
| 661 | DRB1_ | 359 | VLFFKIKDSKIE | 3 | 501 | FKIKDSKI | 0.64 | 0.318492 | 3.49 | <=WB |
| | 0103 | | NPE | | | E | | | | |
| 661 | DRB1_ | 359 | VLFFKIKDSKIE | 3 | 501 | FKIKDSKI | 0.427 | 0.293004 | 4.09 | <=WB |
| | 0405 | | NPE | | | E | | | | |
| 661 | DRB1_ | 359 | VLFFKIKDSKIE | 3 | 501 | FKIKDSKI | 0.993 | 0.440629 | 3.74 | <=WB |
| | 1101 | | NPE | | | E | | | | |
| 678 | DRB1_ | 360 | DYDFRLIDCPE | 3 | 502 | FRLIDCPE | 0.993 | 0.295673 | 4.61 | <=WB |
| | 0401 | | NSFK | | | N | | | | |
| 678 | DRB1_ | 360 | DYDFRLIDCPE | 3 | 502 | FRLIDCPE | 0.993 | 0.392001 | 3.8 | <=WB |
| | 0408 | | NSFK | | | N | | | | |
| 678 | DRB3_ | 360 | DYDFRLIDCPE | 3 | 502 | FRLIDCPE | 0.973 | 0.203751 | 2.29 | <=WB |
| | 0202 | | NSFK | | | N | | | | |
| 678 | DRB1_ | 360 | DYDFRLIDCPE | 3 | 502 | FRLIDCPE | 1 | 0.738732 | 0.85 | <=SB |
| | 1001 | | NSFK | | | N | | | | |
| 678 | DRB1_ | 360 | DYDFRLIDCPE | 3 | 502 | FRLIDCPE | 0.973 | 0.279327 | 4.34 | <=WB |
| | 0405 | | NSFK | | | N | | | | |
| 688 | DRB5_ | 361 | ENSFKDENQNL | 3 | 503 | FKDENQN | 1 | 0.560714 | 1.45 | <=WB |
| | 0101 | | KSNG | | | LK | | | | |
| 688 | DRB1_ | 361 | ENSFKDENQNL | 3 | 503 | FKDENQN | 0.913 | 0.125714 | 3.93 | <=WB |
| | 0305 | | KSNG | | | LK | | | | |
| 695 | DRB3_ | 362 | NQNLKSNGIST | 3 | 504 | LKSNGIST | 1 | 0.403724 | 0.93 | <=SB |
| | 0202 | | SNAS | | | S | | | | |
| 711 | DRB1_ | 363 | HNNYSLDIESES | 3 | 505 | YSLDIESE | 1 | 0.483 | 2.25 | <=WB |
| | 0401 | | SVR | | | S | | | | |
| 711 | DRB1_ | 363 | HNNYSLDIESES | 3 | 505 | YSLDIESE | 0.933 | 0.168357 | 4.32 | <=WB |
| | 1402 | | SVR | | | S | | | | |
| 711 | DRB1_ | 363 | HNNYSLDIESES | 3 | 505 | YSLDIESE | 0.987 | 0.255453 | 1.85 | <=WB |
| | 0305 | | SVR | | | S | | | | |
| 711 | DRB3_ | 363 | HNNYSLDIESES | 3 | 505 | YSLDIESE | 1 | 0.348153 | 1.61 | <=WB |
| | 0101 | | SVR | | | S | | | | |
| 715 | DRB1_ | 364 | SLDIESESSVRTI | 3 | 506 | IESESSVR | 1 | 0.50171 | 2.09 | <=WB |
| | 0401 | | NK | | | T | | | | |
| 715 | DRB1_ | 364 | SLDIESESSVRTI | 3 | 506 | TESESSVR | 1 | 0.367101 | 4.18 | <=WB |
| | 0408 | | NK | | | T | | | | |
| 715 | DRB1_ | 364 | SLDIESESSVRTI | 3 | 506 | IESESSVR | 1 | 0.885269 | 0.45 | <=SB |
| | 0102 | | NK | | | T | | | | |
| 715 | DRB1_ | 364 | SLDIESESSVRTI | 3 | 506 | IESESSVR | 1 | 0.696887 | 1.37 | <=WB |
| | 0101 | | NK | | | T | | | | |
| 715 | DRB1_ | 364 | SLDIESESSVRTI | 3 | 506 | IESESSVR | 1 | 0.307168 | 3.68 | <=WB |
| | 0103 | | NK | | | T | | | | |
| 715 | DRB1_ | 364 | SLDIESESSVRTI | 3 | 506 | IESESSVR | 1 | 0.501522 | 2.69 | <=WB |
| | 0404 | | NK | | | T | | | | |
| 721 | DRB1_ | 365 | ESSVRTINKRV | 3 | 507 | VRTINKR | 0.973 | 0.514903 | 0.89 | <=SB |
| | 1302 | | DPKK | | | VD | | | | |
| 721 | DRB1_ | 365 | ESSVRTINKRV | 3 | 507 | VRTINKR | 1 | 0.943294 | 0.06 | <=SB |
| | 1303 | | DPKK | | | VD | | | | |
| 721 | DRB1_ | 365 | ESSVRTINKRV | 3 | 507 | VRTINKR | 0.98 | 0.209745 | 2.79 | <=WB |
| | 1402 | | DPKK | | | VD | | | | |
| 721 | DRB1_ | 365 | ESSVRTINKRV | 3 | 507 | VRTINKR | 1 | 0.965147 | 0.03 | <=SB |
| | 0801 | | DPKK | | | VD | | | | |
| 721 | DRB1_ | 365 | ESSVRTINKRV | 3 | 507 | VRTINKR | 0.993 | 0.830086 | 0.04 | <=SB |
| | 0803 | | DPKK | | | VD | | | | |
| 721 | DRB1_ | 365 | ESSVRTINKRV | 3 | 507 | VRTINKR | 1 | 0.830796 | 0.78 | <=SB |
| | 1101 | | DPKK | | | VD | | | | |
| 721 | DRB1_ | 365 | ESSVRTINKRV | 3 | 507 | VRTINKR | 1 | 0.904539 | 0.35 | <=SB |
| | 1104 | | DPKK | | | VD | | | | |
| 721 | DRB1_ | 365 | ESSVRTINKRV | 3 | 507 | VRTINKR | 1 | 0.779331 | 0.75 | <=SB |
| | 1301 | | DPKK | | | VD | | | | |
| 721 | DRB1_ | 365 | ESSVRTINKRV | 3 | 507 | VRTINKR | 1 | 0.675567 | 0.58 | <=SB |
| | 1401 | | DPKK | | | VD | | | | |
| 721 | DRB1_ | 365 | ESSVRTINKRV | 3 | 507 | VRTINKR | 1 | 0.675567 | 0.58 | <=SB |
| | 1454 | | DPKK | | | VD | | | | |
| 721 | DRB1_ | 365 | ESSVRTINKRV | 3 | 507 | VRTINKR | 0.967 | 0.338554 | 3.36 | <=WB |
| | 1201 | | DPKK | | | VD | | | | |
| 721 | DRB4_ | 365 | ESSVRTINKRV | 3 | 507 | VRTINKR | 0.893 | 0.143009 | 3.61 | <=WB |
| | 0101 | | DPKK | | | VD | | | | |
| 721 | DRB4_ | 365 | ESSVRTINKRV | 3 | 507 | VRTINKR | 0.893 | 0.143009 | 3.61 | <=WB |
| | 0103 | | DPKK | | | VD | | | | |
| 724 | DRB5_ | 366 | VRTINKRVDPK | 3 | 508 | INKRVDP | 0.953 | 0.242833 | 4.33 | <=WB |
| | 0202 | | KAID | | | KK | | | | |
| 728 | DRB1_ | 367 | NKRVDPKKAID | 3 | 509 | VDPKKAI | 1 | 0.718098 | 0.57 | <=SB |
| | 0103 | | IADK | | | DI | | | | |
| 728 | DRB5_ | 367 | NKRVDPKKAID | 3 | 509 | VDPKKAI | 1 | 0.271013 | 3.72 | <=WB |
| | 0202 | | IADK | | | DI | | | | |
| 734 | DRB1_ | 368 | KKAIDIADKDL | 3 | 510 | IDIADKDL | 0.953 | 0.463709 | 2.78 | <=WB |
| | 1303 | | NFDD | | | N | | | | |
| 734 | DRB1_ | 368 | KKAIDIADKDL | 3 | 510 | IDIADKDL | 0.927 | 0.24839 | 1.82 | <=WB |
| | 1402 | | NFDD | | | N | | | | |
| 734 | DRB4_ | 368 | KKAIDIADKDL | 3 | 510 | IDIADKDL | 0.98 | 0.430635 | 0.55 | <=SB |
| | 0101 | | NFDD | | | N | | | | |
| 734 | DRB4_ | 368 | KKAIDIADKDL | 3 | 510 | IDIADKDL | 0.98 | 0.430635 | 0.55 | <=SB |
| | 0103 | | NFDD | | | N | | | | |
| 734 | DRB3_ | 368 | KKAIDIADKDL | 4 | 511 | DIADKDL | 0.813 | 0.158651 | 3.48 | <=WB |
| | 0101 | | NFDD | | | NF | | | | |
| 734 | DRB1_ | 368 | KKAIDIADKDL | 3 | 510 | IDIADKDL | 0.753 | 0.341572 | 3.33 | <=WB |
| | 0301 | | NFDD | | | N | | | | |
| 734 | DRB1_ | 368 | KKAIDIADKDL | 3 | 510 | IDIADKDL | 0.573 | 0.109819 | 4.44 | <=WB |
| | 0305 | | NFDD | | | N | | | | |
| 734 | DRB1_ | 368 | KKAIDIADKDL | 3 | 510 | IDIADKDL | 0.993 | 0.570137 | 2.66 | <=WB |
| | 1104 | | NFDD | | | N | | | | |
| 734 | DRB1_ | 368 | KKAIDIADKDL | 3 | 510 | IDIADKDL | 0.987 | 0.466083 | 2.07 | <=WB |
| | 1401 | | NFDD | | | N | | | | |
| 734 | DRB1_ | 368 | KKAIDIADKDL | 3 | 510 | IDIADKDL | 0.987 | 0.466083 | 2.07 | <=WB |
| | 1454 | | NFDD | | | N | | | | |
| 735 | DRB1_ | 369 | KAIDIADKDLN | 3 | 511 | DIADKDL | 0.553 | 0.247253 | 4.56 | <=WB |
| | 0301 | | FDDD | | | NF | | | | |

Table 3 below shows predicted human MHCII binding of peptides derived from BB0838 determined using NetMHCIIpan (V 4.0). The prediction outcome for each molecule includes the following columns in the table: Pos Residue number (staffing from 22); MHC molecule name; Peptide Amino acid sequence; Of Starting position offset of the optimal binding core (staffing from 0); Core Binding core register; Core_Rel Reliability of the binding core, expressed as the fraction of networks in the ensemble selecting the optimal core; Score_EL Eluted ligand prediction score; % Rank_EL Percentile rank of eluted ligand prediction score; BindLevel (SB: strong binder, WB: weak binder). The peptide will be identified as a strong binder if the % Rank is below the 1% threshold for the strong binders. The peptide will be identified as a weak binder if the 0 Rank is above the 1% threshold of the strong binders but below the 50% threshold for the weak binders.

TABLE 3
Predicted human CD4+ T cell epitopes from B. burgdorferi LptD

| | | SEQ | | | SEQ | | | | | % | |
|---|---|---|---|---|---|---|---|---|---|---|---|
| | | ID | Pep- | | ID | | Core_ | | Score_ | Rank_ | Bind |
| Pos | MHC | NO: | tide | Of | NO: | Core | Rel | Identity | EL | EL | Level |
| 22 | DRB1_ | 512 | TFSNAI | 5 | 1098 | IFAQTI | 0.973 | Sequence | 0.675879 | 1.21 | <= |
| | 0403 | | FAQTID | | | DDE | | | | | WB |
| | | | DEN | | | | | | | | |
| 23 | DRB1_ | 513 | FSNAIF | 4 | 1098 | IFAQTI | 0.933 | Sequence | 0.808567 | 0.44 | <= |
| | 0403 | | AQTIDD | | | DDE | | | | | SB |
| | | | ENS | | | | | | | | |
| 23 | DRB1_ | 513 | FSNAIF | 4 | 1098 | IFAQTI | 0.613 | Sequence | 0.347018 | 3.28 | <= |
| | 0405 | | AQTIDD | | | DDE | | | | | WB |
| | | | ENS | | | | | | | | |
| 24 | DRB1_ | 514 | SNAIFA | 4 | 1099 | FAQTID | 0.567 | Sequence | 0.403065 | 3 | <= |
| | 0401 | | QTIDDE | | | DEN | | | | | WB |
| | | | NSK | | | | | | | | |
| 24 | DRB1_ | 514 | SNAIFA | 3 | 1098 | IFAQTI | 0.893 | Sequence | 0.878489 | 0.18 | <= |
| | 0403 | | QTIDDE | | | DDE | | | | | SB |
| | | | NSK | | | | | | | | |
| 24 | DRB1_ | 514 | SNAIFA | 3 | 1098 | IFAQTI | 0.793 | Sequence | 0.374027 | 4.22 | <= |
| | 0404 | | QTIDDE | | | DDE | | | | | WB |
| | | | NSK | | | | | | | | |
| 24 | DRB1_ | 514 | SNAIFA | 3 | 1098 | IFAQTI | 0.513 | Sequence | 0.509055 | 1.8 | <= |
| | 0405 | | QTIDDE | | | DDE | | | | | WB |
| | | | NSK | | | | | | | | |
| 24 | DRB1_ | 514 | SNAIFA | 4 | 1099 | FAQTID | 0.587 | Sequence | 0.410671 | 3.54 | <= |
| | 0408 | | QTIDDE | | | DEN | | | | | WB |
| | | | NSK | | | | | | | | |
| 25 | DRB1_ | 515 | NAIFAQ | 3 | 1099 | FAQTID | 0.88 | Sequence | 0.467892 | 2.37 | <= |
| | 0401 | | TIDDEN | | | DEN | | | | | WB |
| | | | SKK | | | | | | | | |
| 25 | DRB1_ | 515 | NAIFAQ | 2 | 1098 | IFAQTI | 0.627 | Sequence | 0.820142 | 0.39 | <= |
| | 0403 | | TIDDEN | | | DDE | | | | | SB |
| | | | SKK | | | | | | | | |
| 25 | DRB1_ | 515 | NAIFAQ | 3 | 1099 | FAQTID | 0.8 | Sequence | 0.505845 | 1.82 | <= |
| | 0405 | | TIDDEN | | | DEN | | | | | WB |
| | | | SKK | | | | | | | | |
| 25 | DRB1_ | 515 | NAIFAQ | 3 | 1099 | FAQTID | 0.9 | Sequence | 0.492494 | 2.64 | <= |
| | 0408 | | TIDDEN | | | DEN | | | | | WB |
| | | | SKK | | | | | | | | |
| 26 | DRB1_ | 516 | AIFAQT | 2 | 1099 | FAQTID | 0.527 | Sequence | 0.525139 | 2.62 | <= |
| | 0403 | | IDDENS | | | DEN | | | | | WB |
| | | | KKR | | | | | | | | |
| 28 | DRB1_ | 517 | FAQTID | 4 | 1100 | IDDENS | 0.733 | Sequence | 0.30424 | 3.74 | <= |
| | 0301 | | DENSKK | | | KKR | | | | | WB |
| | | | RDK | | | | | | | | |
| 29 | DRB1_ | 518 | AQTIDD | 3 | 1100 | IDDENS | 0.833 | Sequence | 0.270896 | 4.2 | <= |
| | 0301 | | ENSKKR | | | KKR | | | | | WB |
| | | | DKL | | | | | | | | |
| 37 | DRB1_ | 519 | SKKRDK | 6 | 1101 | LTLSQK | 0.993 | Sequence | 0.329959 | 4.37 | <= |
| | 1401 | | LTLSQK | | | SYL | | | | | WB |
| | | | SYL | | | | | | | | |
| 37 | DRB1_ | 519 | SKKRDK | 6 | 1101 | LTLSQK | 0.993 | Sequence | 0.329959 | 4.37 | <= |
| | 1454 | | LTLSQK | | | SYL | | | | | WB |
| | | | SYL | | | | | | | | |
| 38 | DRB1_ | 520 | KKRDKL | 5 | 1101 | LTLSQK | 1 | Sequence | 0.742155 | 0.36 | <= |
| | 1401 | | TLSQKS | | | SYL | | | | | SB |
| | | | YLR | | | | | | | | |
| 38 | DRB1_ | 520 | KKRDKL | 5 | 1101 | LTLSQK | 1 | Sequence | 0.742155 | 0.36 | <= |
| | 1454 | | TLSQKS | | | SYL | | | | | SB |
| | | | YLR | | | | | | | | |
| 39 | DRB1_ | 521 | KRDKLT | 4 | 1101 | LTLSQK | 1 | Sequence | 0.824408 | 0.14 | <= |
| | 1401 | | LSQKSY | | | SYL | | | | | SB |
| | | | LRE | | | | | | | | |
| 39 | DRB1_ | 521 | KRDKLT | 4 | 1101 | LTLSQK | 1 | Sequence | 0.824408 | 0.14 | <= |
| | 1454 | | LSQKSY | | | SYL | | | | | SB |
| | | | LRE | | | | | | | | |
| 39 | DRB1_ | 521 | KRDKLT | 4 | 1101 | LTLSQK | 0.973 | Sequence | 0.159141 | 4.79 | <= |
| | 1402 | | LSQKSY | | | SYL | | | | | WB |
| | | | LRE | | | | | | | | |
| 39 | DRB1_ | 521 | KRDKLT | 4 | 1101 | LTLSQK | 1 | Sequence | 0.310313 | 4.52 | <= |
| | 1302 | | LSQKSY | | | SYL | | | | | WB |
| | | | LRE | | | | | | | | |
| 40 | DRB1_ | 522 | RDKLTL | 3 | 1101 | LTLSQK | 1 | Sequence | 0.782031 | 0.24 | <= |
| | 1401 | | SQKSYL | | | SYL | | | | | SB |
| | | | REL | | | | | | | | |
| 40 | DRB1_ | 522 | RDKLTL | 3 | 1101 | LTLSQK | 1 | Sequence | 0.782031 | 0.24 | <= |
| | 1454 | | SQKSYL | | | SYL | | | | | SB |
| | | | REL | | | | | | | | |
| 41 | DRB1_ | 523 | DKLTLS | 2 | 1101 | LTLSQK | 1 | Sequence | 0.528893 | 1.48 | <= |
| | 1401 | | QKSYLR | | | SYL | | | | | WB |
| | | | ELE | | | | | | | | |
| 41 | DRB1_ | 523 | DKLTLS | 2 | 1101 | LTLSQK | 1 | Sequence | 0.528893 | 1.48 | <= |
| | 1454 | | QKSYLR | | | SYL | | | | | WB |
| | | | ELE | | | | | | | | |
| 51 | DRB1_ | 524 | LRELEL | 5 | 1102 | LSTDED | 0.967 | Sequence | 0.328262 | 3.45 | <= |
| | 0301 | | STDEDL | | | LKK | | | | | WB |
| | | | KKW | | | | | | | | |
| 52 | DRB1_ | 525 | RELELS | 4 | 1102 | LSTDED | 0.993 | Sequence | 0.498135 | 2.05 | <= |
| | 0301 | | TDEDLK | | | LKK | | | | | WB |
| | | | KWA | | | | | | | | |
| 52 | DRB1_ | 525 | RELELS | 4 | 1102 | LSTDED | 0.933 | Sequence | 0.100768 | 4.8 | <= |
| | 0305 | | TDEDLK | | | LKK | | | | | WB |
| | | | KWA | | | | | | | | |
| 53 | DRB1_ | 526 | ELELST | 3 | 1102 | LSTDED | 1 | Sequence | 0.458779 | 2.33 | <= |
| | 0301 | | DEDLKK | | | LKK | | | | | WB |
| | | | WAL | | | | | | | | |
| 58 | DRB1_ | 527 | TDEDLK | 4 | 1103 | LKKWAL | 0.987 | Sequence | 0.268725 | 4.61 | <= |
| | 0402 | | KWALKE | | | KEG | | | | | WB |
| | | | GLK | | | | | | | | |
| 59 | DRB1_ | 528 | DEDLKK | 3 | 1103 | LKKWAL | 0.993 | Sequence | 0.330541 | 3.42 | <= |
| | 0402 | | WALKEG | | | KEG | | | | | WB |
| | | | LKE | | | | | | | | |
| 60 | DRB1_ | 529 | EDLKKW | 5 | 1104 | WALKEG | 0.953 | Sequence | 0.531277 | 3.09 | <= |
| | 0801 | | ALKEGL | | | LKE | | | | | WB |
| | | | KET | | | | | | | | |
| 60 | DRB1_ | 529 | EDLKKW | 5 | 1104 | WALKEG | 0.907 | Sequence | 0.285458 | 4.95 | <= |
| | 0803 | | ALKEGL | | | LKE | | | | | WB |
| | | | KET | | | | | | | | |
| 61 | DRB1_ | 530 | DLKKWA | 4 | 1104 | WALKEG | 0.987 | Sequence | 0.65556 | 1.67 | <= |
| | 0801 | | LKEGLK | | | LKE | | | | | WB |
| | | | ETD | | | | | | | | |
| 61 | DRB1_ | 530 | DLKKWA | 4 | 1104 | WALKEG | 0.987 | Sequence | 0.390485 | 2.4 | <= |
| | 0803 | | LKEGLK | | | LKE | | | | | WB |
| | | | ETD | | | | | | | | |
| 62 | DRB1_ | 531 | LKKWAL | 3 | 1104 | WALKEG | 0.98 | Sequence | 0.641486 | 1.8 | <= |
| | 0801 | | KEGLKE | | | LKE | | | | | WB |
| | | | TDV | | | | | | | | |
| 62 | DRB1_ | 531 | LKKWAL | 3 | 1104 | WALKEG | 0.967 | Sequence | 0.391671 | 2.38 | <= |
| | 0803 | | KEGLKE | | | LKE | | | | | WB |
| | | | TDV | | | | | | | | |
| 63 | DRB1_ | 532 | KKWALK | 2 | 1104 | WALKEG | 0.893 | Sequence | 0.542665 | 2.93 | <= |
| | 0801 | | EGLKET | | | LKE | | | | | WB |
| | | | DVS | | | | | | | | |
| 63 | DRB1_ | 532 | KKWALK | 2 | 1104 | WALKEG | 0.9 | Sequence | 0.290318 | 4.79 | <= |
| | 0803 | | EGLKET | | | LKE | | | | | WB |
| | | | DVS | | | | | | | | |
| 66 | DRB1_ | 533 | ALKEGL | 5 | 1105 | LKETDV | 1 | Sequence | 0.551351 | 1.44 | <= |
| | 0701 | | KETDVS | | | SKI | | | | | WB |
| | | | KIR | | | | | | | | |
| 67 | DRB1_ | 534 | LKEGLK | 4 | 1105 | LKETDV | 1 | Sequence | 0.6913 | 0.75 | <= |
| | 0701 | | ETDVSK | | | SKI | | | | | SB |
| | | | IRE | | | | | | | | |
| 67 | DRB1_ | 534 | LKEGLK | 4 | 1105 | LKETDV | 1 | Sequence | 0.452541 | 2.93 | <= |
| | 0901 | | ETDVSK | | | SKI | | | | | WB |
| | | | IRE | | | | | | | | |
| 67 | DRB1_ | 534 | LKEGLK | 4 | 1105 | LKETDV | 0.987 | Sequence | 0.333979 | 4.27 | <= |
| | 1401 | | ETDVSK | | | SKI | | | | | WB |
| | | | IRE | | | | | | | | |
| 67 | DRB1_ | 534 | LKEGLK | 4 | 1105 | LKETDV | 0.987 | Sequence | 0.333979 | 4.27 | <= |
| | 1454 | | ETDVSK | | | SKI | | | | | WB |
| | | | IRE | | | | | | | | |
| 68 | DRB1_ | 535 | KEGLKE | 3 | 1105 | LKETDV | 1 | Sequence | 0.645093 | 0.93 | <= |
| | 0701 | | TDVSKI | | | SKI | | | | | SB |
| | | | REL | | | | | | | | |
| 68 | DRB1_ | 535 | KEGLKE | 3 | 1105 | LKETDV | 1 | Sequence | 0.41475 | 3.59 | <= |
| | 0901 | | TDVSKI | | | SKI | | | | | WB |
| | | | REL | | | | | | | | |
| 69 | DRB1_ | 536 | EGLKET | 2 | 1105 | LKETDV | 1 | Sequence | 0.277464 | 4.25 | <= |
| | 0701 | | DVSKIR | | | SKI | | | | | WB |
| | | | ELL | | | | | | | | |
| 71 | DRB1_ | 537 | LKETDV | 5 | 1106 | VSKIRE | 0.993 | Sequence | 0.404587 | 2.32 | <= |
| | 1201 | | SKIREL | | | LLL | | | | | WB |
| | | | LLK | | | | | | | | |
| 72 | DRB1_ | 538 | KETDVS | 4 | 1106 | VSKIRE | 1 | Sequence | 0.592009 | 0.81 | <= |
| | 1201 | | KIRELL | | | LLL | | | | | SB |
| | | | LKK | | | | | | | | |
| 72 | DRB1_ | 538 | KETDVS | 4 | 1106 | VSKIRE | 0.98 | Sequence | 0.216585 | 3.73 | <= |
| | 1503 | | KIRELL | | | LLL | | | | | WB |
| | | | LKK | | | | | | | | |
| 73 | DRB1_ | 539 | ETDVSK | 3 | 1106 | VSKIRE | 0.807 | Sequence | 0.627453 | 0.65 | <= |
| | 1201 | | IRELLL | | | LLL | | | | | SB |
| | | | KKF | | | | | | | | |
| 73 | DRB1_ | 539 | ETDVSK | 3 | 1106 | VSKIRE | 0.987 | Sequence | 0.218812 | 3.68 | <= |
| | 1503 | | IRELLL | | | LLL | | | | | WB |
| | | | KKF | | | | | | | | |
| 74 | DRB1_ | 540 | TDVSKI | 5 | 1107 | IRELLL | 0.64 | Sequence | 0.586207 | 0.84 | <= |
| | 1201 | | RELLLK | | | KKF | | | | | SB |
| | | | KFG | | | | | | | | |
| 75 | DRB1_ | 541 | DVSKIR | 4 | 1107 | IRELLL | 0.9 | Sequence | 0.532715 | 1.13 | <= |
| | 1201 | | ELLLKK | | | KKF | | | | | WB |
| | | | FGI | | | | | | | | |
| 76 | DRB1_ | 542 | VSKIRE | 3 | 1107 | IRELLL | 0.9 | Sequence | 0.61865 | 0.69 | <= |
| | 1201 | | LLLKKF | | | KKF | | | | | SB |
| | | | GID | | | | | | | | |
| 77 | DRB1_ | 543 | SKIREL | 2 | 1107 | IRELLL | 0.8 | Sequence | 0.494779 | 1.4 | <= |
| | 1201 | | LLKKFG | | | KKF | | | | | WB |
| | | | IDP | | | | | | | | |
| 78 | DRB1_ | 544 | KIRELL | 1 | 1107 | IRELLL | 0.527 | Sequence | 0.295635 | 4.31 | <= |
| | 1201 | | LKKFGI | | | KKF | | | | | WB |
| | | | DPE | | | | | | | | |
| 81 | DRB1_ | 545 | ELLLKK | 6 | 1108 | FGIDPE | 0.98 | Sequence | 0.102382 | 4.74 | <= |
| | 0305 | | FGIDPE | | | LFV | | | | | WB |
| | | | LFV | | | | | | | | |
| 82 | DRB1_ | 546 | LLLKKF | 5 | 1108 | FGIDPE | 1 | Sequence | 0.370515 | 3.06 | <= |
| | 0301 | | GIDPEL | | | LFV | | | | | WB |
| | | | FVK | | | | | | | | |
| 82 | DRB1_ | 546 | LLLKKF | 5 | 1108 | FGIDPE | 1 | Sequence | 0.443904 | 0.72 | <= |
| | 0305 | | GIDPEL | | | LFV | | | | | SB |
| | | | FVK | | | | | | | | |
| 83 | DRB1_ | 547 | LLKKFG | 4 | 1108 | FGIDPE | 1 | Sequence | 0.538345 | 1.79 | <= |
| | 0301 | | IDPELF | | | LFV | | | | | WB |
| | | | VKG | | | | | | | | |
| 83 | DRB1_ | 547 | LLKKFG | 4 | 1108 | FGIDPE | 1 | Sequence | 0.581148 | 0.31 | <= |
| | 0305 | | IDPELF | | | LFV | | | | | SB |
| | | | VKG | | | | | | | | |
| 83 | DRB1_ | 547 | LLKKFG | 4 | 1108 | FGIDPE | 0.967 | Sequence | 0.216735 | 2.59 | <= |
| | 1402 | | IDPELF | | | LFV | | | | | WB |
| | | | VKG | | | | | | | | |
| 83 | DRB1_ | 547 | LLKKFG | 4 | 1108 | FGIDPE | 1 | Sequence | 0.404181 | 3.45 | <= |
| | 1303 | | IDPELF | | | LFV | | | | | WB |
| | | | VKG | | | | | | | | |
| 84 | DRB1_ | 548 | LKKFGI | 3 | 1108 | FGIDPE | 1 | Sequence | 0.651027 | 1.22 | <= |
| | 0301 | | DPELFV | | | LFV | | | | | WB |
| | | | KGK | | | | | | | | |
| 84 | DRB1_ | 548 | LKKFGI | 3 | 1108 | FGIDPE | 1 | Sequence | 0.675455 | 0.16 | <= |
| | 0305 | | DPELFV | | | LFV | | | | | SB |
| | | | KGK | | | | | | | | |
| 84 | DRB1_ | 548 | LKKFGI | 3 | 1108 | FGIDPE | 1 | Sequence | 0.352046 | 3.67 | <= |
| | 0401 | | DPELFV | | | LFV | | | | | WB |
| | | | KGK | | | | | | | | |
| 84 | DRB1_ | 548 | LKKFGI | 3 | 1108 | FGIDPE | 0.98 | Sequence | 0.315068 | 0.96 | <= |
| | 1402 | | DPELFV | | | LFV | | | | | SB |
| | | | KGK | | | | | | | | |
| 84 | DRB1_ | 548 | LKKFGI | 3 | 1108 | FGIDPE | 1 | Sequence | 0.516052 | 2.24 | <= |
| | 1303 | | DPELFV | | | LFV | | | | | WB |
| | | | KGK | | | | | | | | |
| 84 | DRB1_ | 548 | LKKFGI | 3 | 1108 | FGIDPE | 0.96 | Sequence | 0.321168 | 4.19 | <= |
| | 1302 | | DPELFV | | | LFV | | | | | WB |
| | | | KGK | | | | | | | | |
| 85 | DRB1_ | 549 | KKFGID | 2 | 1108 | FGIDPE | 1 | Sequence | 0.525862 | 1.87 | <= |
| | 0301 | | PELFVK | | | LFV | | | | | WB |
| | | | GKG | | | | | | | | |
| 85 | DRB1_ | 549 | KKFGID | 2 | 1108 | FGIDPE | 1 | Sequence | 0.558209 | 0.36 | <= |
| | 0305 | | PELFVK | | | LFV | | | | | SB |
| | | | GKG | | | | | | | | |
| 85 | DRB1_ | 549 | KKFGID | 2 | 1108 | FGIDPE | 0.953 | Sequence | 0.199647 | 3.1 | <= |
| | 1402 | | PELFVK | | | LFV | | | | | WB |
| | | | GKG | | | | | | | | |
| 85 | DRB1_ | 549 | KKFGID | 2 | 1108 | FGIDPE | 0.993 | Sequence | 0.372019 | 3.89 | <= |
| | 1303 | | PELFVK | | | LFV | | | | | WB |
| | | | GKG | | | | | | | | |
| 86 | DRB1_ | 550 | KFGIDP | 1 | 1108 | FGIDPE | 0.98 | Sequence | 0.17166 | 2.89 | <= |
| | 0305 | | ELFVKG | | | LFV | | | | | WB |
| | | | KGL | | | | | | | | |
| 88 | DRB1_ | 551 | GIDPEL | 5 | 1109 | LFVKGK | 0.993 | Sequence | 0.625065 | 2 | <= |
| | 1101 | | FVKGKG | | | GLA | | | | | WB |
| | | | LAG | | | | | | | | |
| 88 | DRB1_ | 551 | GIDPEL | 5 | 1109 | LFVKGK | 0.973 | Sequence | 0.730195 | 1.34 | <= |
| | 1104 | | FVKGKG | | | GLA | | | | | WB |
| | | | LAG | | | | | | | | |
| 88 | DRB1_ | 551 | GIDPEL | 5 | 1109 | LFVKGK | 0.973 | Sequence | 0.563218 | 2.77 | <= |
| | 1301 | | FVKGKG | | | GLA | | | | | WB |
| | | | LAG | | | | | | | | |
| 89 | DRB1_ | 552 | IDPELF | 5 | 1110 | FVKGKG | 0.907 | Sequence | 0.353896 | 3.88 | <= |
| | 0101 | | VKGKGL | | | LAG | | | | | WB |
| | | | AGS | | | | | | | | |
| 89 | DRB1_ | 552 | IDPELF | 5 | 1110 | FVKGKG | 0.82 | Sequence | 0.365767 | 2.84 | <= |
| | 0103 | | VKGKGL | | | LAG | | | | | WB |
| | | | AGS | | | | | | | | |
| 89 | DRB1_ | 552 | IDPELF | 4 | 1109 | LFVKGK | 0.867 | Sequence | 0.432719 | 4.8 | <= |
| | 0801 | | VKGKGL | | | GLA | | | | | WB |
| | | | AGS | | | | | | | | |
| 89 | DRB1_ | 552 | IDPELF | 4 | 1109 | LFVKGK | 0.987 | Sequence | 0.763619 | 1.15 | <= |
| | 1101 | | VKGKGL | | | GLA | | | | | WB |
| | | | AGS | | | | | | | | |
| 89 | DRB1_ | 552 | IDPELF | 4 | 1109 | LFVKGK | 1 | Sequence | 0.841944 | 0.66 | <= |
| | 1104 | | VKGKGL | | | GLA | | | | | SB |
| | | | AGS | | | | | | | | |
| 89 | DRB1_ | 552 | IDPELF | 4 | 1109 | LFVKGK | 0.993 | Sequence | 0.690092 | 1.4 | <= |
| | 1301 | | VKGKGL | | | GLA | | | | | WB |
| | | | AGS | | | | | | | | |
| 89 | DRB1_ | 552 | IDPELF | 4 | 1109 | LFVKGK | 0.88 | Sequence | 0.318946 | 4.26 | <= |
| | 1302 | | VKGKGL | | | GLA | | | | | WB |
| | | | AGS | | | | | | | | |
| 90 | DRB1_ | 553 | DPELFV | 4 | 1110 | FVKGKG | 1 | Sequence | 0.590555 | 1.93 | <= |
| | 0101 | | KGKGLA | | | LAG | | | | | WB |
| | | | GSG | | | | | | | | |
| 90 | DRB1_ | 553 | DPELFV | 4 | 1110 | FVKGKG | 0.94 | Sequence | 0.382439 | 4.42 | <= |
| | 0102 | | KGKGLA | | | LAG | | | | | WB |
| | | | GSG | | | | | | | | |
| 90 | DRB1_ | 553 | DPELFV | 4 | 1110 | FVKGKG | 0.967 | Sequence | 0.564411 | 1.19 | <= |
| | 0103 | | KGKGLA | | | LAG | | | | | WB |
| | | | GSG | | | | | | | | |
| 90 | DRB1_ | 553 | DPELFV | 3 | 1109 | LFVKGK | 0.82 | Sequence | 0.492668 | 3.68 | <= |
| | 0801 | | KGKGLA | | | GLA | | | | | WB |
| | | | GSG | | | | | | | | |
| 90 | DRB1_ | 553 | DPELFV | 4 | 1110 | FVKGKG | 0.933 | Sequence | 0.361124 | 4.74 | <= |
| | 0901 | | KGKGLA | | | LAG | | | | | WB |
| | | | GSG | | | | | | | | |
| 90 | DRB1_ | 553 | DPELFV | 4 | 1110 | FVKGKG | 0.98 | Sequence | 0.41589 | 4.34 | <= |
| | 1001 | | KGKGLA | | | LAG | | | | | WB |
| | | | GSG | | | | | | | | |
| 90 | DRB1_ | 553 | DPELFV | 3 | 1109 | LFVKGK | 0.94 | Sequence | 0.824641 | 0.82 | <= |
| | 1101 | | KGKGLA | | | GLA | | | | | SB |
| | | | GSG | | | | | | | | |
| 90 | DRB1_ | 553 | DPELFV | 3 | 1109 | LFVKGK | 1 | Sequence | 0.88344 | 0.45 | <= |
| | 1104 | | KGKGLA | | | GLA | | | | | SB |
| | | | GSG | | | | | | | | |
| 90 | DRB1_ | 553 | DPELFV | 3 | 1109 | LFVKGK | 0.993 | Sequence | 0.738334 | 1.01 | <= |
| | 1301 | | KGKGLA | | | GLA | | | | | WB |
| | | | GSG | | | | | | | | |
| 90 | DRB1_ | 553 | DPELFV | 4 | 1110 | FVKGKG | 0.913 | Sequence | 0.365899 | 4.22 | <= |
| | 1601 | | KGKGLA | | | LAG | | | | | WB |
| | | | GSG | | | | | | | | |
| 90 | DRB1_ | 553 | DPELFV | 3 | 1109 | LFVKGK | 0.867 | Sequence | 0.370924 | 2.84 | <= |
| | 1302 | | KGKGLA | | | GLA | | | | | WB |
| | | | GSG | | | | | | | | |
| 91 | DRB1_ | 554 | PELFVK | 3 | 1110 | FVKGKG | 1 | Sequence | 0.712948 | 1.28 | <= |
| | 0101 | | GKGLAG | | | LAG | | | | | WB |
| | | | SGR | | | | | | | | |
| 91 | DRB1_ | 554 | PELFVK | 3 | 1110 | FVKGKG | 0.993 | Sequence | 0.50175 | 3.05 | <= |
| | 0102 | | GKGLAG | | | LAG | | | | | WB |
| | | | SGR | | | | | | | | |
| 91 | DRB1_ | 554 | PELFVK | 3 | 1110 | FVKGKG | 0.993 | Sequence | 0.700074 | 0.62 | <= |
| | 0103 | | GKGLAG | | | LAG | | | | | SB |
| | | | SGR | | | | | | | | |
| 91 | DRB1_ | 554 | PELFVK | 3 | 1110 | FVKGKG | 1 | Sequence | 0.316644 | 4.24 | <= |
| | 0401 | | GKGLAG | | | LAG | | | | | WB |
| | | | SGR | | | | | | | | |
| 91 | DRB1_ | 554 | PELFVK | 3 | 1110 | FVKGKG | 1 | Sequence | 0.417896 | 3.53 | <= |
| | 0901 | | GKGLAG | | | LAG | | | | | WB |
| | | | SGR | | | | | | | | |
| 91 | DRB1_ | 554 | PELFVK | 3 | 1110 | FVKGKG | 1 | Sequence | 0.519953 | 2.76 | <= |
| | 1001 | | GKGLAG | | | LAG | | | | | WB |
| | | | SGR | | | | | | | | |
| 91 | DRB1_ | 554 | PELFVK | 2 | 1109 | LFVKGK | 0.62 | Sequence | 0.687364 | 1.6 | <= |
| | 1101 | | GKGLAG | | | GLA | | | | | WB |
| | | | SGR | | | | | | | | |
| 91 | DRB1_ | 554 | PELFVK | 2 | 1109 | LFVKGK | 0.907 | Sequence | 0.705773 | 1.52 | <= |
| | 1104 | | GKGLAG | | | GLA | | | | | WB |
| | | | SGR | | | | | | | | |
| 91 | DRB1_ | 554 | PELFVK | 2 | 1109 | LFVKGK | 0.9 | Sequence | 0.547108 | 2.96 | <= |
| | 1301 | | GKGLAG | | | GLA | | | | | WB |
| | | | SGR | | | | | | | | |
| 91 | DRB1_ | 554 | PELFVK | 3 | 1110 | FVKGKG | 0.993 | Sequence | 0.476714 | 2.77 | <= |
| | 1601 | | GKGLAG | | | LAG | | | | | WB |
| | | | SGR | | | | | | | | |
| 91 | DRB1_ | 554 | PELFVK | 3 | 1110 | FVKGKG | 0.793 | Sequence | 0.169217 | 4.28 | <= |
| | 1402 | | GKGLAG | | | LAG | | | | | WB |
| | | | SGR | | | | | | | | |
| 92 | DRB1_ | 555 | ELFVKG | 2 | 1110 | FVKGKG | 1 | Sequence | 0.373184 | 3.67 | <= |
| | 0101 | | KGLAGS | | | LAG | | | | | WB |
| | | | GRY | | | | | | | | |
| 92 | DRB1_ | 555 | ELFVKG | 2 | 1110 | FVKGKG | 0.967 | Sequence | 0.392863 | 2.53 | <= |
| | 0103 | | KGLAGS | | | LAG | | | | | WB |
| | | | GRY | | | | | | | | |
| 95 | DRB1_ | 556 | VKGKGL | 5 | 1111 | LAGSGR | 0.993 | Sequence | 0.520717 | 1.55 | <= |
| | 1401 | | AGSGRY | | | YKI | | | | | WB |
| | | | KII | | | | | | | | |
| 95 | DRB1_ | 556 | VKGKGL | 5 | 1111 | LAGSGR | 0.993 | Sequence | 0.520717 | 1.55 | <= |
| | 1454 | | AGSGRY | | | YKI | | | | | WB |
| | | | KII | | | | | | | | |
| 96 | DRB1_ | 557 | KGKGLA | 4 | 1111 | LAGSGR | 1 | Sequence | 0.582985 | 1.06 | <= |
| | 1401 | | GSGRYK | | | YKI | | | | | WB |
| | | | III | | | | | | | | |
| 96 | DRB1_ | 557 | KGKGLA | 4 | 1111 | LAGSGR | 1 | Sequence | 0.582985 | 1.06 | <= |
| | 1454 | | GSGRYK | | | YKI | | | | | WB |
| | | | III | | | | | | | | |
| 97 | DRB1_ | 558 | GKGLAG | 3 | 1111 | LAGSGR | 1 | Sequence | 0.645286 | 0.71 | <= |
| | 1401 | | SGRYKI | | | YKI | | | | | SB |
| | | | IIE | | | | | | | | |
| 97 | DRB1_ | 558 | GKGLAG | 3 | 1111 | LAGSGR | 1 | Sequence | 0.645286 | 0.71 | <= |
| | 1454 | | SGRYKI | | | YKI | | | | | SB |
| | | | IIE | | | | | | | | |
| 98 | DRB1_ | 559 | KGLAGS | 2 | 1111 | LAGSGR | 1 | Sequence | 0.358331 | 3.73 | <= |
| | 1401 | | GRYKII | | | YKI | | | | | WB |
| | | | IET | | | | | | | | |
| 98 | DRB1_ | 559 | KGLAGS | 2 | 1111 | LAGSGR | 1 | Sequence | 0.358331 | 3.73 | <= |
| | 1454 | | GRYKII | | | YKI | | | | | WB |
| | | | IET | | | | | | | | |
| 100 | DRB1_ | 560 | LAGSGR | 6 | 1112 | YKIIIE | 1 | Sequence | 0.383872 | 2.85 | <= |
| | 0405 | | YKIIIE | | | TAD | | | | | WB |
| | | | TAD | | | | | | | | |
| 100 | DRB1_ | 560 | LAGSGR | 6 | 1112 | YKIIIE | 1 | Sequence | 0.600926 | 2.23 | <= |
| | 0801 | | YKIIIE | | | TAD | | | | | WB |
| | | | TAD | | | | | | | | |
| 100 | DRB1_ | 560 | LAGSGR | 6 | 1112 | YKIIIE | 1 | Sequence | 0.352524 | 4.17 | <= |
| | 1303 | | YKIIIE | | | TAD | | | | | WB |
| | | | TAD | | | | | | | | |
| 100 | DRB1_ | 560 | LAGSGR | 6 | 1112 | YKIIIE | 0.98 | Sequence | 0.438283 | 1.71 | <= |
| | 0803 | | YKIIIE | | | TAD | | | | | WB |
| | | | TAD | | | | | | | | |
| 101 | DRB1_ | 561 | AGSGRY | 5 | 1112 | YKIIIE | 0.993 | Sequence | 0.681634 | 1.17 | <= |
| | 0403 | | KIIIET | | | TAD | | | | | WB |
| | | | ADN | | | | | | | | |
| 101 | DRB1_ | 561 | AGSGRY | 5 | 1112 | YKIIIE | 1 | Sequence | 0.820369 | 0.42 | <= |
| | 0405 | | KIIIET | | | TAD | | | | | SB |
| | | | ADN | | | | | | | | |
| 101 | DRB1_ | 561 | AGSGRY | 5 | 1112 | YKIIIE | 1 | Sequence | 0.895028 | 0.2 | <= |
| | 0801 | | KIIIET | | | TAD | | | | | SB |
| | | | ADN | | | | | | | | |
| 101 | DRB1_ | 561 | AGSGRY | 5 | 1112 | YKIIIE | 0.947 | Sequence | 0.472975 | 3.39 | <= |
| | 1001 | | KIIIET | | | TAD | | | | | WB |
| | | | ADN | | | | | | | | |
| 101 | DRB1_ | 561 | AGSGRY | 5 | 1112 | YKIIIE | 0.927 | Sequence | 0.160347 | 4.73 | <= |
| | 1402 | | KIIIET | | | TAD | | | | | WB |
| | | | ADN | | | | | | | | |
| 101 | DRB1_ | 561 | AGSGRY | 5 | 1112 | YKIIIE | 1 | Sequence | 0.738918 | 0.77 | <= |
| | 1303 | | KIIIET | | | TAD | | | | | SB |
| | | | ADN | | | | | | | | |
| 101 | DRB1_ | 561 | AGSGRY | 5 | 1112 | YKIIIE | 1 | Sequence | 0.751026 | 0.11 | <= |
| | 0803 | | KIIIET | | | TAD | | | | | SB |
| | | | ADN | | | | | | | | |
| 101 | DRB1_ | 561 | AGSGRY | 5 | 1112 | YKIIIE | 0.993 | Sequence | 0.361238 | 4.27 | <= |
| | 0408 | | KIIIET | | | TAD | | | | | WB |
| | | | ADN | | | | | | | | |
| 102 | DRB1_ | 562 | GSGRYK | 4 | 1112 | YKIIIE | 0.98 | Sequence | 0.704101 | 1.03 | <= |
| | 0403 | | IIIETA | | | TAD | | | | | WB |
| | | | DNL | | | | | | | | |
| 102 | DRB1_ | 562 | GSGRYK | 4 | 1112 | YKIIIE | 1 | Sequence | 0.833788 | 0.38 | <= |
| | 0405 | | IIIETA | | | TAD | | | | | SB |
| | | | DNL | | | | | | | | |
| 102 | DRB1_ | 562 | GSGRYK | 4 | 1112 | YKIIIE | 1 | Sequence | 0.90077 | 0.18 | <= |
| | 0801 | | IIIETA | | | TAD | | | | | SB |
| | | | DNL | | | | | | | | |
| 102 | DRB1_ | 562 | GSGRYK | 4 | 1112 | YKIIIE | 0.927 | Sequence | 0.516893 | 2.79 | <= |
| | 1001 | | IIIETA | | | TAD | | | | | WB |
| | | | DNL | | | | | | | | |
| 102 | DRB1_ | 562 | GSGRYK | 4 | 1112 | YKIIIE | 0.993 | Sequence | 0.374953 | 4.54 | <= |
| | 1101 | | IIIETA | | | TAD | | | | | WB |
| | | | DNL | | | | | | | | |
| 102 | DRB1_ | 562 | GSGRYK | 4 | 1112 | YKIIIE | 0.813 | Sequence | 0.192556 | 3.35 | <= |
| | 1402 | | IIIETA | | | TAD | | | | | WB |
| | | | DNL | | | | | | | | |
| 102 | DRB1_ | 562 | GSGRYK | 4 | 1112 | YKIIIE | 1 | Sequence | 0.763623 | 0.67 | <= |
| | 1303 | | IIIETA | | | TAD | | | | | SB |
| | | | DNL | | | | | | | | |
| 102 | DRB1_ | 562 | GSGRYK | 4 | 1112 | YKIIIE | 1 | Sequence | 0.765177 | 0.1 | <= |
| | 0803 | | IIIETA | | | TAD | | | | | SB |
| | | | DNL | | | | | | | | |
| 102 | DRB1_ | 562 | GSGRYK | 4 | 1112 | YKIIIE | 0.967 | Sequence | 0.403187 | 3.64 | <= |
| | 0408 | | IIIETA | | | TAD | | | | | WB |
| | | | DNL | | | | | | | | |
| 103 | DRB1_ | 563 | SGRYKI | 5 | 1113 | IIIETA | 0.933 | Sequence | 0.568202 | 2.07 | <= |
| | 0101 | | IIETAD | | | DNL | | | | | WB |
| | | | NLE | | | | | | | | |
| 103 | DRB1_ | 563 | SGRYKI | 5 | 1113 | IIIETA | 1 | Sequence | 0.785372 | 0.97 | <= |
| | 0102 | | IIETAD | | | DNL | | | | | SB |
| | | | NLE | | | | | | | | |
| 103 | DRB1_ | 563 | SGRYKI | 5 | 1113 | IIIETA | 0.587 | Sequence | 0.110579 | 4.41 | <= |
| | 0305 | | IIETAD | | | DNL | | | | | WB |
| | | | NLE | | | | | | | | |
| 103 | DRB1_ | 563 | SGRYKI | 3 | 1112 | YKIIIE | 0.687 | Sequence | 0.399175 | 3.04 | <= |
| | 0401 | | IIETAD | | | TAD | | | | | WB |
| | | | NLE | | | | | | | | |
| 103 | DRB1_ | 563 | SGRYKI | 3 | 1112 | YKIIIE | 0.913 | Sequence | 0.77203 | 0.63 | <= |
| | 0403 | | IIETAD | | | TAD | | | | | SB |
| | | | NLE | | | | | | | | |
| 103 | DRB1_ | 563 | SGRYKI | 3 | 1112 | YKIIIE | 0.5 | Sequence | 0.3405 | 4.75 | <= |
| | 0404 | | TIETAD | | | TAD | | | | | WB |
| | | | NLE | | | | | | | | |
| 103 | DRB1_ | 563 | SGRYKI | 3 | 1112 | YKIIIE | 0.993 | Sequence | 0.878826 | 0.25 | <= |
| | 0405 | | IIETAD | | | TAD | | | | | SB |
| | | | NLE | | | | | | | | |
| 103 | DRB1_ | 563 | SGRYKI | 3 | 1112 | YKIIIE | 0.733 | Sequence | 0.262529 | 4.51 | <= |
| | 0701 | | IIETAD | | | TAD | | | | | WB |
| | | | NLE | | | | | | | | |
| 103 | DRB1_ | 563 | SGRYKI | 3 | 1112 | YKIIIE | 1 | Sequence | 0.930667 | 0.09 | <= |
| | 0801 | | IIETAD | | | TAD | | | | | SB |
| | | | NLE | | | | | | | | |
| 103 | DRB1_ | 563 | SGRYKI | 3 | 1112 | YKIIIE | 0.627 | Sequence | 0.352337 | 4.97 | <= |
| | 0901 | | IIETAD | | | TAD | | | | | WB |
| | | | NLE | | | | | | | | |
| 103 | DRB1_ | 563 | SGRYKI | 3 | 1112 | YKIIIE | 0.833 | Sequence | 0.638395 | 1.57 | <= |
| | 1001 | | IIETAD | | | TAD | | | | | WB |
| | | | NLE | | | | | | | | |
| 103 | DRB1_ | 563 | SGRYKI | 3 | 1112 | YKIIIE | 0.993 | Sequence | 0.478396 | 3.31 | <= |
| | 1101 | | IIETAD | | | TAD | | | | | WB |
| | | | NLE | | | | | | | | |
| 103 | DRB1_ | 563 | SGRYKI | 5 | 1113 | IIIETA | 0.76 | Sequence | 0.39728 | 2.41 | <= |
| | 1201 | | IIETAD | | | DNL | | | | | WB |
| | | | NLE | | | | | | | | |
| 103 | DRB1_ | 563 | SGRYKI | 5 | 1113 | IIIETA | 0.62 | Sequence | 0.346741 | 3.97 | <= |
| | 1401 | | IIETAD | | | DNL | | | | | WB |
| | | | NLE | | | | | | | | |
| 103 | DRB1_ | 563 | SGRYKI | 5 | 1113 | IIIETA | 0.927 | Sequence | 0.206596 | 4.84 | <= |
| | 1501 | | IIETAD | | | DNL | | | | | WB |
| | | | NLE | | | | | | | | |
| 103 | DRB1_ | 563 | SGRYKI | 3 | 1112 | YKIIIE | 0.56 | Sequence | 0.401503 | 3.71 | <= |
| | 1601 | | IIETAD | | | TAD | | | | | WB |
| | | | NLE | | | | | | | | |
| 103 | DRB1_ | 563 | SGRYKI | 5 | 1113 | IIIETA | 0.88 | Sequence | 0.174088 | 4.85 | <= |
| | 1503 | | IIETAD | | | DNL | | | | | WB |
| | | | NLE | | | | | | | | |
| 103 | DRB1_ | 563 | SGRYKI | 5 | 1113 | IIIETA | 0.62 | Sequence | 0.346741 | 3.97 | <= |
| | 1454 | | IIETAD | | | DNL | | | | | WB |
| | | | NLE | | | | | | | | |
| 103 | DRB1_ | 563 | SGRYKI | 3 | 1112 | YKIIIE | 0.667 | Sequence | 0.275244 | 1.41 | <= |
| | 1402 | | IIETAD | | | TAD | | | | | WB |
| | | | NLE | | | | | | | | |
| 103 | DRB1_ | 563 | SGRYKI | 3 | 1112 | YKIIIE | 1 | Sequence | 0.82199 | 0.44 | <= |
| | 1303 | | IIETAD | | | TAD | | | | | SB |
| | | | NLE | | | | | | | | |
| 103 | DRB1_ | 563 | SGRYKI | 5 | 1113 | IIIETA | 0.66 | Sequence | 0.305513 | 4.69 | <= |
| | 1302 | | IIETAD | | | DNL | | | | | WB |
| | | | NLE | | | | | | | | |
| 103 | DRB1_ | 563 | SGRYKI | 3 | 1112 | YKIIIE | 0.993 | Sequence | 0.81369 | 0.05 | <= |
| | 0803 | | IIETAD | | | TAD | | | | | SB |
| | | | NLE | | | | | | | | |
| 103 | DRB1_ | 563 | SGRYKI | 3 | 1112 | YKIIIE | 0.84 | Sequence | 0.53464 | 2.22 | <= |
| | 0408 | | IIETAD | | | TAD | | | | | WB |
| | | | NLE | | | | | | | | |
| 104 | DRB1_ | 564 | GRYKII | 4 | 1113 | IIIETA | 1 | Sequence | 0.796135 | 0.89 | <= |
| | 0101 | | IETADN | | | DNL | | | | | SB |
| | | | LEN | | | | | | | | |
| 104 | DRB1_ | 564 | GRYKII | 4 | 1113 | IIIETA | 1 | Sequence | 0.908032 | 0.35 | <= |
| | 0102 | | IETADN | | | DNL | | | | | SB |
| | | | LEN | | | | | | | | |
| 104 | DRB1_ | 564 | GRYKII | 4 | 1113 | IIIETA | 0.973 | Sequence | 0.378991 | 2.69 | <= |
| | 0103 | | IETADN | | | DNL | | | | | WB |
| | | | LEN | | | | | | | | |
| 104 | DRB1_ | 564 | GRYKII | 4 | 1113 | IIIETA | 0.98 | Sequence | 0.332237 | 3.41 | <= |
| | 0301 | | IETADN | | | DNL | | | | | WB |
| | | | LEN | | | | | | | | |
| 104 | DRB1_ | 564 | GRYKII | 4 | 1113 | IIIETA | 0.78 | Sequence | 0.145013 | 3.43 | <= |
| | 0305 | | IETADN | | | DNL | | | | | WB |
| | | | LEN | | | | | | | | |
| 104 | DRB1_ | 564 | GRYKII | 4 | 1113 | IIIETA | 0.713 | Sequence | 0.370403 | 3.41 | <= |
| | 0401 | | IETADN | | | DNL | | | | | WB |
| | | | LEN | | | | | | | | |
| 104 | DRB1_ | 564 | GRYKII | 5 | 1114 | IIETAD | 0.52 | Sequence | 0.797652 | 0.5 | <= |
| | 0403 | | IETADN | | | NLE | | | | | SB |
| | | | LEN | | | | | | | | |
| 104 | DRB1_ | 564 | GRYKII | 4 | 1113 | IIIETA | 0.627 | Sequence | 0.459641 | 3.12 | <= |
| | 0404 | | IETADN | | | DNL | | | | | WB |
| | | | LEN | | | | | | | | |
| 104 | DRB1_ | 564 | GRYKII | 2 | 1112 | YKIIIE | 0.62 | Sequence | 0.772096 | 0.58 | <= |
| | 0405 | | IETADN | | | TAD | | | | | SB |
| | | | LEN | | | | | | | | |
| 104 | DRB1_ | 564 | GRYKII | 2 | 1112 | YKIIIE | 0.967 | Sequence | 0.826909 | 0.48 | <= |
| | 0801 | | IETADN | | | TAD | | | | | SB |
| | | | LEN | | | | | | | | |
| 104 | DRB1_ | 564 | GRYKII | 4 | 1113 | IITETA | 0.533 | Sequence | 0.544591 | 2.46 | <= |
| | 1001 | | IETADN | | | DNL | | | | | WB |
| | | | LEN | | | | | | | | |
| 104 | DRB1_ | 564 | GRYKII | 4 | 1113 | IIIETA | 0.92 | Sequence | 0.475356 | 1.55 | <= |
| | 1201 | | IETADN | | | DNL | | | | | WB |
| | | | LEN | | | | | | | | |
| 104 | DRB1_ | 564 | GRYKII | 4 | 1113 | IIIETA | 0.793 | Sequence | 0.397667 | 2.98 | <= |
| | 1401 | | IETADN | | | DNL | | | | | WB |
| | | | LEN | | | | | | | | |
| 104 | DRB1_ | 564 | GRYKII | 4 | 1113 | IIIETA | 0.98 | Sequence | 0.371832 | 2.59 | <= |
| | 1501 | | IETADN | | | DNL | | | | | WB |
| | | | LEN | | | | | | | | |
| 104 | DRB1_ | 564 | GRYKII | 4 | 1113 | IIIETA | 0.847 | Sequence | 0.463306 | 2.93 | <= |
| | 1601 | | IETADN | | | DNL | | | | | WB |
| | | | LEN | | | | | | | | |
| 104 | DRB1_ | 564 | GRYKII | 4 | 1113 | IIIETA | 0.94 | Sequence | 0.307994 | 2.33 | <= |
| | 1503 | | IETADN | | | DNL | | | | | WB |
| | | | LEN | | | | | | | | |
| 104 | DRB1_ | 564 | GRYKII | 4 | 1113 | IIIETA | 0.793 | Sequence | 0.397667 | 2.98 | <= |
| | 1454 | | IETADN | | | DNL | | | | | WB |
| | | | LEN | | | | | | | | |
| 104 | DRB1_ | 564 | GRYKII | 4 | 1113 | IIIETA | 0.633 | Sequence | 0.245935 | 1.87 | <= |
| | 1402 | | IETADN | | | DNL | | | | | WB |
| | | | LEN | | | | | | | | |
| 104 | DRB1_ | 564 | GRYKII | 2 | 1112 | YKIIIE | 0.92 | Sequence | 0.634934 | 1.33 | <= |
| | 1303 | | IETADN | | | TAD | | | | | WB |
| | | | LEN | | | | | | | | |
| 104 | DRB1_ | 564 | GRYKII | 4 | 1113 | IIIETA | 0.887 | Sequence | 0.373594 | 2.79 | <= |
| | 1302 | | IETADN | | | DNL | | | | | WB |
| | | | LEN | | | | | | | | |
| 104 | DRB1_ | 564 | GRYKII | 2 | 1112 | YKIIIE | 0.887 | Sequence | 0.670853 | 0.27 | <= |
| | 0803 | | IETADN | | | TAD | | | | | SB |
| | | | LEN | | | | | | | | |
| 104 | DRB1_ | 564 | GRYKII | 4 | 1113 | IIIETA | 0.467 | Sequence | 0.370798 | 4.11 | <= |
| | 0408 | | IETADN | | | DNL | | | | | WB |
| | | | LEN | | | | | | | | |
| 105 | DRB1_ | 565 | RYKIII | 3 | 1113 | IIIETA | 1 | Sequence | 0.790917 | 0.92 | <= |
| | 0101 | | ETADNL | | | DNL | | | | | SB |
| | | | ENF | | | | | | | | |
| 105 | DRB1_ | 565 | RYKIII | 3 | 1113 | IIIETA | 1 | Sequence | 0.903268 | 0.37 | <= |
| | 0102 | | ETADNL | | | DNL | | | | | SB |
| | | | ENF | | | | | | | | |
| 105 | DRB1_ | 565 | RYKIII | 3 | 1113 | IIIETA | 0.987 | Sequence | 0.363132 | 2.87 | <= |
| | 0103 | | ETADNL | | | DNL | | | | | WB |
| | | | ENF | | | | | | | | |
| 105 | DRB1_ | 565 | RYKIII | 3 | 1113 | IIIETA | 0.953 | Sequence | 0.320787 | 3.52 | <= |
| | 0301 | | ETADNL | | | DNL | | | | | WB |
| | | | ENF | | | | | | | | |
| 105 | DRB1_ | 565 | RYKIII | 3 | 1113 | IIIETA | 0.787 | Sequence | 0.132946 | 3.73 | <= |
| | 0305 | | ETADNL | | | DNL | | | | | WB |
| | | | ENF | | | | | | | | |
| 105 | DRB1_ | 565 | RYKIII | 3 | 1113 | IIIETA | 0.627 | Sequence | 0.376137 | 3.33 | <= |
| | 0401 | | ETADNL | | | DNL | | | | | WB |
| | | | ENF | | | | | | | | |
| 105 | DRB1_ | 565 | RYKIII | 3 | 1113 | IIIETA | 0.48 | Sequence | 0.263592 | 4.73 | <= |
| | 0402 | | ETADNL | | | DNL | | | | | WB |
| | | | ENF | | | | | | | | |
| 105 | DRB1_ | 565 | RYKIII | 4 | 1114 | IIETAD | 0.733 | Sequence | 0.802145 | 0.47 | <= |
| | 0403 | | ETADNL | | | NLE | | | | | SB |
| | | | ENF | | | | | | | | |
| 105 | DRB1_ | 565 | RYKIII | 3 | 1113 | IIIETA | 0.353 | Sequence | 0.568231 | 2.1 | <= |
| | 0404 | | ETADNL | | | DNL | | | | | WB |
| | | | ENF | | | | | | | | |
| 105 | DRB1_ | 565 | RYKIII | 4 | 1114 | IIETAD | 0.847 | Sequence | 0.667768 | 0.99 | <= |
| | 0405 | | ETADNL | | | NLE | | | | | SB |
| | | | ENF | | | | | | | | |
| 105 | DRB1_ | 565 | RYKIII | 1 | 1112 | YKIIIE | 0.787 | Sequence | 0.43687 | 4.72 | <= |
| | 0801 | | ETADNL | | | TAD | | | | | WB |
| | | | ENF | | | | | | | | |
| 105 | DRB1_ | 565 | RYKIII | 3 | 1113 | IIIETA | 0.76 | Sequence | 0.408399 | 4.47 | <= |
| | 1001 | | ETADNL | | | DNL | | | | | WB |
| | | | ENF | | | | | | | | |
| 105 | DRB1_ | 565 | RYKIII | 3 | 1113 | IIIETA | 0.947 | Sequence | 0.461862 | 1.67 | <= |
| | 1201 | | ETADNL | | | DNL | | | | | WB |
| | | | ENF | | | | | | | | |
| 105 | DRB1_ | 565 | RYKIII | 3 | 1113 | IIIETA | 0.76 | Sequence | 0.365838 | 3.58 | <= |
| | 1401 | | ETADNL | | | DNL | | | | | WB |
| | | | ENF | | | | | | | | |
| 105 | DRB1_ | 565 | RYKIII | 3 | 1113 | IIIETA | 0.96 | Sequence | 0.362791 | 2.67 | <= |
| | 1501 | | ETADNL | | | DNL | | | | | WB |
| | | | ENF | | | | | | | | |
| 105 | DRB1_ | 565 | RYKIII | 3 | 1113 | IIIETA | 0.953 | Sequence | 0.432066 | 3.32 | <= |
| | 1601 | | ETADNL | | | DNL | | | | | WB |
| | | | ENF | | | | | | | | |
| 105 | DRB1_ | 565 | RYKIII | 3 | 1113 | IITETA | 0.913 | Sequence | 0.297537 | 2.44 | <= |
| | 1503 | | ETADNL | | | DNL | | | | | WB |
| | | | ENF | | | | | | | | |
| 105 | DRB1_ | 565 | RYKIII | 3 | 1113 | IIIETA | 0.76 | Sequence | 0.365838 | 3.58 | <= |
| | 1454 | | ETADNL | | | DNL | | | | | WB |
| | | | ENF | | | | | | | | |
| 105 | DRB1_ | 565 | RYKIII | 3 | 1113 | IIIETA | 0.74 | Sequence | 0.199515 | 3.11 | <= |
| | 1402 | | ETADNL | | | DNL | | | | | WB |
| | | | ENF | | | | | | | | |
| 105 | DRB1_ | 565 | RYKIII | 3 | 1113 | IIIETA | 0.907 | Sequence | 0.338313 | 3.68 | <= |
| | 1302 | | ETADNL | | | DNL | | | | | WB |
| | | | ENF | | | | | | | | |
| 105 | DRB1_ | 565 | RYKIII | 1 | 1112 | YKIIIE | 0.507 | Sequence | 0.359885 | 2.94 | <= |
| | 0803 | | ETADNL | | | TAD | | | | | WB |
| | | | ENF | | | | | | | | |
| 105 | DRB1_ | 565 | RYKIII | 3 | 1113 | IIIETA | 0.36 | Sequence | 0.383954 | 3.91 | <= |
| | 0408 | | ETADNL | | | DNL | | | | | WB |
| | | | ENF | | | | | | | | |
| 106 | DRB1_ | 566 | YKIIIE | 2 | 1113 | IIIETA | 0.993 | Sequence | 0.430219 | 3.13 | <= |
| | 0101 | | TADNLE | | | DNL | | | | | WB |
| | | | NFT | | | | | | | | |
| 106 | DRB1_ | 566 | YKIIIE | 2 | 1113 | IIIETA | 0.987 | Sequence | 0.693824 | 1.51 | <= |
| | 0102 | | TADNLE | | | DNL | | | | | WB |
| | | | NFT | | | | | | | | |
| 106 | DRB1_ | 566 | YKIIIE | 4 | 1115 | IETADN | 0.66 | Sequence | 0.368238 | 3.43 | <= |
| | 0401 | | TADNLE | | | LEN | | | | | WB |
| | | | NFT | | | | | | | | |
| 106 | DRB1_ | 566 | YKIIIE | 3 | 1114 | IIETAD | 0.487 | Sequence | 0.259885 | 4.81 | <= |
| | 0402 | | TADNLE | | | NLE | | | | | WB |
| | | | NFT | | | | | | | | |
| 106 | DRB1_ | 566 | YKIIIE | 3 | 1114 | IIETAD | 0.793 | Sequence | 0.822819 | 0.38 | <= |
| | 0403 | | TADNLE | | | NLE | | | | | SB |
| | | | NFT | | | | | | | | |
| 106 | DRB1_ | 566 | YKIIIE | 4 | 1115 | IETADN | 0.613 | Sequence | 0.647432 | 1.52 | <= |
| | 0404 | | TADNLE | | | LEN | | | | | WB |
| | | | NFT | | | | | | | | |
| 106 | DRB1_ | 566 | YKIIIE | 3 | 1114 | IIETAD | 0.92 | Sequence | 0.689682 | 0.9 | <= |
| | 0405 | | TADNLE | | | NLE | | | | | SB |
| | | | NFT | | | | | | | | |
| 106 | DRB1_ | 566 | YKIIIE | 2 | 1113 | IIIETA | 0.787 | Sequence | 0.276881 | 4.81 | <= |
| | 1201 | | TADNLE | | | DNL | | | | | WB |
| | | | NFT | | | | | | | | |
| 106 | DRB1_ | 566 | YKIIIE | 4 | 1115 | IETADN | 0.613 | Sequence | 0.442432 | 3.17 | <= |
| | 0408 | | TADNLE | | | LEN | | | | | WB |
| | | | NFT | | | | | | | | |
| 107 | DRB1_ | 567 | KIIIET | 3 | 1115 | IETADN | 0.913 | Sequence | 0.361851 | 3.52 | <= |
| | 0401 | | ADNLEN | | | LEN | | | | | WB |
| | | | FTY | | | | | | | | |
| 107 | DRB1_ | 567 | KIIIET | 2 | 1114 | IIETAD | 0.547 | Sequence | 0.695868 | 1.07 | <= |
| | 0403 | | ADNLEN | | | NLE | | | | | WB |
| | | | FTY | | | | | | | | |
| 107 | DRB1_ | 567 | KIIIET | 3 | 1115 | IETADN | 0.9 | Sequence | 0.618342 | 1.73 | <= |
| | 0404 | | ADNLEN | | | LEN | | | | | WB |
| | | | FTY | | | | | | | | |
| 107 | DRB1_ | 567 | KIIIET | 2 | 1114 | IIETAD | 0.76 | Sequence | 0.462334 | 2.13 | <= |
| | 0405 | | ADNLEN | | | NLE | | | | | WB |
| | | | FTY | | | | | | | | |
| 107 | DRB1_ | 567 | KIIIET | 3 | 1115 | IETADN | 0.893 | Sequence | 0.40985 | 3.55 | <= |
| | 0408 | | ADNLEN | | | LEN | | | | | WB |
| | | | FTY | | | | | | | | |
| 116 | DRB1_ | 568 | LENFTY | 5 | 1116 | YGLTKD | 0.98 | Sequence | 0.534712 | 1.55 | <= |
| | 0701 | | GLTKDE | | | ESI | | | | | WB |
| | | | SII | | | | | | | | |
| 117 | DRB1_ | 569 | ENFTYG | 4 | 1116 | YGLTKD | 0.993 | Sequence | 0.600513 | 1.16 | <= |
| | 0701 | | LTKDES | | | ESI | | | | | WB |
| | | | IIF | | | | | | | | |
| 118 | DRB1_ | 570 | NFTYGL | 5 | 1117 | LTKDES | 1 | Sequence | 0.415741 | 2.67 | <= |
| | 0301 | | TKDESI | | | IIF | | | | | WB |
| | | | IFE | | | | | | | | |
| 118 | DRB1_ | 570 | NFTYGL | 5 | 1117 | LTKDES | 0.967 | Sequence | 0.183674 | 2.7 | <= |
| | 0305 | | TKDESI | | | IIF | | | | | WB |
| | | | IFE | | | | | | | | |
| 118 | DRB1_ | 570 | NFTYGL | 3 | 1116 | YGLTKD | 1 | Sequence | 0.687288 | 0.77 | <= |
| | 0701 | | TKDESI | | | ESI | | | | | SB |
| | | | IFE | | | | | | | | |
| 118 | DRB1_ | 570 | NFTYGL | 3 | 1116 | YGLTKD | 0.967 | Sequence | 0.417105 | 3.54 | <= |
| | 0901 | | TKDESI | | | ESI | | | | | WB |
| | | | IFE | | | | | | | | |
| 119 | DRB1_ | 571 | FTYGLT | 4 | 1117 | LTKDES | 1 | Sequence | 0.63759 | 1.27 | <= |
| | 0301 | | KDESII | | | IIF | | | | | WB |
| | | | FEG | | | | | | | | |
| 119 | DRB1_ | 571 | FTYGLT | 4 | 1117 | LTKDES | 0.987 | Sequence | 0.31956 | 1.32 | <= |
| | 0305 | | KDESII | | | IIF | | | | | WB |
| | | | FEG | | | | | | | | |
| 119 | DRB1_ | 571 | FTYGLT | 2 | 1116 | YGLTKD | 0.967 | Sequence | 0.307838 | 3.76 | <= |
| | 0701 | | KDESII | | | ESI | | | | | WB |
| | | | FEG | | | | | | | | |
| 120 | DRB1_ | 572 | TYGLTK | 3 | 1117 | LTKDES | 1 | Sequence | 0.771114 | 0.69 | <= |
| | 0301 | | DESIIF | | | IIF | | | | | SB |
| | | | EGR | | | | | | | | |
| 120 | DRB1_ | 572 | TYGLTK | 3 | 1117 | LTKDES | 0.993 | Sequence | 0.42797 | 0.78 | <= |
| | 0305 | | DESIIF | | | IIF | | | | | SB |
| | | | EGR | | | | | | | | |
| 121 | DRB1_ | 573 | YGLTKD | 2 | 1117 | LTKDES | 1 | Sequence | 0.502092 | 2.03 | <= |
| | 0301 | | ESIIFE | | | IIF | | | | | WB |
| | | | GRV | | | | | | | | |
| 121 | DRB1_ | 573 | YGLTKD | 2 | 1117 | LTKDES | 1 | Sequence | 0.20513 | 2.41 | <= |
| | 0305 | | ESIIFE | | | IIF | | | | | WB |
| | | | GRV | | | | | | | | |
| 123 | DRB1_ | 574 | LTKDES | 6 | 1118 | IIFEGR | 1 | Sequence | 0.509124 | 1.66 | <= |
| | 1401 | | IIFEGR | | | VNI | | | | | WB |
| | | | VNI | | | | | | | | |
| 123 | DRB1_ | 574 | LTKDES | 6 | 1118 | IIFEGR | 1 | Sequence | 0.509124 | 1.66 | <= |
| | 1454 | | IIFEGR | | | VNI | | | | | WB |
| | | | VNI | | | | | | | | |
| 124 | DRB1_ | 575 | TKDESI | 5 | 1118 | IIFEGR | 1 | Sequence | 0.248405 | 4.54 | <= |
| | 0301 | | IFEGRV | | | VNI | | | | | WB |
| | | | NIL | | | | | | | | |
| 124 | DRB1_ | 575 | TKDESI | 5 | 1118 | IIFEGR | 1 | Sequence | 0.618121 | 0.85 | <= |
| | 1401 | | IFEGRV | | | VNI | | | | | SB |
| | | | NIL | | | | | | | | |
| 124 | DRB1_ | 575 | TKDESI | 5 | 1118 | IIFEGR | 1 | Sequence | 0.618121 | 0.85 | <= |
| | 1454 | | IFEGRV | | | VNI | | | | | SB |
| | | | NIL | | | | | | | | |
| 124 | DRB1_ | 575 | TKDESI | 5 | 1118 | IIFEGR | 0.967 | Sequence | 0.158213 | 4.84 | <= |
| | 1402 | | IFEGRV | | | VNI | | | | | WB |
| | | | NIL | | | | | | | | |
| 125 | DRB1_ | 576 | KDESII | 4 | 1118 | IIFEGR | 1 | Sequence | 0.300524 | 3.79 | <= |
| | 0301 | | FEGRVN | | | VNI | | | | | WB |
| | | | ILV | | | | | | | | |
| 125 | DRB1_ | 576 | KDESII | 4 | 1118 | IIFEGR | 0.993 | Sequence | 0.626592 | 0.81 | <= |
| | 1401 | | FEGRVN | | | VNI | | | | | SB |
| | | | ILV | | | | | | | | |
| 125 | DRB1_ | 576 | KDESII | 4 | 1118 | IIFEGR | 0.993 | Sequence | 0.626592 | 0.81 | <= |
| | 1454 | | FEGRVN | | | VNI | | | | | SB |
| | | | ILV | | | | | | | | |
| 125 | DRB1_ | 576 | KDESII | 4 | 1118 | IIFEGR | 0.947 | Sequence | 0.20251 | 3 | <= |
| | 1402 | | FEGRVN | | | VNI | | | | | WB |
| | | | ILV | | | | | | | | |
| 126 | DRB1_ | 577 | DESIIF | 3 | 1118 | IIFEGR | 1 | Sequence | 0.401067 | 2.8 | <= |
| | 0301 | | EGRVNI | | | VNI | | | | | WB |
| | | | LVE | | | | | | | | |
| 126 | DRB1_ | 577 | DESIIF | 3 | 1118 | IIFEGR | 0.927 | Sequence | 0.115787 | 4.24 | <= |
| | 0305 | | EGRVNI | | | VNI | | | | | WB |
| | | | LVE | | | | | | | | |
| 126 | DRB1_ | 577 | DESIIF | 3 | 1118 | IIFEGR | 0.987 | Sequence | 0.697328 | 0.5 | <= |
| | 1401 | | EGRVNI | | | VNI | | | | | SB |
| | | | LVE | | | | | | | | |
| 126 | DRB1_ | 577 | DESIIF | 3 | 1118 | IIFEGR | 0.987 | Sequence | 0.697328 | 0.5 | <= |
| | 1454 | | EGRVNI | | | VNI | | | | | SB |
| | | | LVE | | | | | | | | |
| 126 | DRB1_ | 577 | DESIIF | 3 | 1118 | IIFEGR | 0.927 | Sequence | 0.26296 | 1.59 | <= |
| | 1402 | | EGRVNI | | | VNI | | | | | WB |
| | | | LVE | | | | | | | | |
| 126 | DRB1_ | 577 | DESIIF | 3 | 1118 | IIFEGR | 0.827 | Sequence | 0.38143 | 2.63 | <= |
| | 1302 | | EGRVNI | | | VNI | | | | | WB |
| | | | LVE | | | | | | | | |
| 127 | DRB1_ | 578 | ESIIFE | 4 | 1119 | FEGRVN | 0.947 | Sequence | 0.334451 | 3.26 | <= |
| | 0103 | | GRVNIL | | | ILV | | | | | WB |
| | | | VED | | | | | | | | |
| 127 | DRB1_ | 578 | ESIIFE | 2 | 1118 | IIFEGR | 0.88 | Sequence | 0.457142 | 2.18 | <= |
| | 1401 | | GRVNIL | | | VNI | | | | | WB |
| | | | VED | | | | | | | | |
| 127 | DRB1_ | 578 | ESIIFE | 2 | 1118 | IIFEGR | 0.88 | Sequence | 0.457142 | 2.18 | <= |
| | 1454 | | GRVNIL | | | VNI | | | | | WB |
| | | | VED | | | | | | | | |
| 128 | DRB1_ | 579 | SIIFEG | 3 | 1119 | FEGRVN | 0.98 | Sequence | 0.293075 | 3.9 | <= |
| | 0103 | | RVNILV | | | ILV | | | | | WB |
| | | | EDI | | | | | | | | |
| 132 | DRB1_ | 580 | EGRVNI | 6 | 1120 | LVEDIK | 0.987 | Sequence | 0.584019 | 1.55 | <= |
| | 0301 | | LVEDIK | | | ENK | | | | | WB |
| | | | ENK | | | | | | | | |
| 132 | DRB1_ | 580 | EGRVNI | 6 | 1120 | LVEDIK | 0.84 | Sequence | 0.123511 | 3.98 | <= |
| | 0305 | | LVEDIK | | | ENK | | | | | WB |
| | | | ENK | | | | | | | | |
| 132 | DRB1_ | 580 | EGRVNI | 3 | 1121 | VNILVE | 0.787 | Sequence | 0.270585 | 4.99 | <= |
| | 1201 | | LVEDIK | | | DIK | | | | | WB |
| | | | ENK | | | | | | | | |
| 133 | DRB1_ | 581 | GRVNIL | 5 | 1120 | LVEDIK | 1 | Sequence | 0.915047 | 0.21 | <= |
| | 0301 | | VEDIKE | | | ENK | | | | | SB |
| | | | NKK | | | | | | | | |
| 133 | DRB1_ | 581 | GRVNIL | 5 | 1120 | LVEDIK | 0.98 | Sequence | 0.413262 | 0.84 | <= |
| | 0305 | | VEDIKE | | | ENK | | | | | SB |
| | | | NKK | | | | | | | | |
| 133 | DRB1_ | 581 | GRVNIL | 4 | 1122 | ILVEDI | 0.96 | Sequence | 0.421434 | 4.24 | <= |
| | 1001 | | VEDIKE | | | KEN | | | | | WB |
| | | | NKK | | | | | | | | |
| 134 | DRB1_ | 582 | RVNILV | 4 | 1120 | LVEDIK | 1 | Sequence | 0.952679 | 0.08 | <= |
| | 0301 | | EDIKEN | | | ENK | | | | | SB |
| | | | KKH | | | | | | | | |
| 134 | DRB1_ | 582 | RVNILV | 4 | 1120 | LVEDIK | 0.973 | Sequence | 0.526745 | 0.44 | <= |
| | 0305 | | EDIKEN | | | ENK | | | | | SB |
| | | | KKH | | | | | | | | |
| 134 | DRB1_ | 582 | RVNILV | 3 | 1122 | ILVEDI | 0.96 | Sequence | 0.295809 | 4.01 | <= |
| | 0402 | | EDIKEN | | | KEN | | | | | WB |
| | | | KKH | | | | | | | | |
| 134 | DRB1_ | 582 | RVNILV | 3 | 1122 | ILVEDI | 0.993 | Sequence | 0.494531 | 3.08 | <= |
| | 1001 | | EDIKEN | | | KEN | | | | | WB |
| | | | KKH | | | | | | | | |
| 134 | DRB1_ | 582 | RVNILV | 4 | 1120 | LVEDIK | 0.493 | Sequence | 0.162973 | 4.58 | <= |
| | 1402 | | EDIKEN | | | ENK | | | | | WB |
| | | | KKH | | | | | | | | |
| 135 | DRB1_ | 583 | VNILVE | 3 | 1120 | LVEDIK | 1 | Sequence | 0.962246 | 0.05 | <= |
| | 0301 | | DIKENK | | | ENK | | | | | SB |
| | | | KHN | | | | | | | | |
| 135 | DRB1_ | 583 | VNILVE | 3 | 1120 | LVEDIK | 0.987 | Sequence | 0.570707 | 0.34 | <= |
| | 0305 | | DIKENK | | | ENK | | | | | SB |
| | | | KHN | | | | | | | | |
| 136 | DRB1_ | 584 | NILVED | 2 | 1120 | LVEDIK | 1 | Sequence | 0.908527 | 0.23 | <= |
| | 0301 | | IKENKK | | | ENK | | | | | SB |
| | | | HNI | | | | | | | | |
| 136 | DRB1_ | 584 | NILVED | 2 | 1120 | LVEDIK | 0.947 | Sequence | 0.397753 | 0.9 | <= |
| | 0305 | | IKENKK | | | ENK | | | | | SB |
| | | | HNI | | | | | | | | |
| 136 | DRB1_ | 584 | NILVED | 6 | 1123 | IKENKK | 0.68 | Sequence | 0.512041 | 3.48 | <= |
| | 1301 | | IKENKK | | | HNI | | | | | WB |
| | | | HNI | | | | | | | | |
| 136 | DRB1_ | 584 | NILVED | 6 | 1123 | IKENKK | 0.82 | Sequence | 0.342011 | 4.08 | <= |
| | 1401 | | IKENKK | | | HNI | | | | | WB |
| | | | HNI | | | | | | | | |
| 136 | DRB1_ | 584 | NILVED | 6 | 1123 | IKENKK | 0.82 | Sequence | 0.342011 | 4.08 | <= |
| | 1454 | | IKENKK | | | HNI | | | | | WB |
| | | | HNI | | | | | | | | |
| 136 | DRB1_ | 584 | NILVED | 6 | 1123 | IKENKK | 0.867 | Sequence | 0.471697 | 1.31 | <= |
| | 1302 | | IKENKK | | | HNI | | | | | WB |
| | | | HNI | | | | | | | | |
| 137 | DRB1_ | 585 | ILVEDI | 1 | 1120 | LVEDIK | 0.793 | Sequence | 0.60523 | 1.44 | <= |
| | 0301 | | KENKKH | | | ENK | | | | | WB |
| | | | NIK | | | | | | | | |
| 137 | DRB1_ | 585 | ILVEDI | 5 | 1123 | IKENKK | 0.467 | Sequence | 0.153163 | 3.25 | <= |
| | 0305 | | KENKKH | | | HNI | | | | | WB |
| | | | NIK | | | | | | | | |
| 137 | DRB1_ | 585 | ILVEDI | 5 | 1123 | IKENKK | 0.987 | Sequence | 0.560515 | 2.77 | <= |
| | 1104 | | KENKKH | | | HNI | | | | | WB |
| | | | NIK | | | | | | | | |
| 137 | DRB1_ | 585 | ILVEDI | 5 | 1123 | IKENKK | 1 | Sequence | 0.79234 | 0.67 | <= |
| | 1301 | | KENKKH | | | HNI | | | | | SB |
| | | | NIK | | | | | | | | |
| 137 | DRB1_ | 585 | ILVEDI | 5 | 1123 | IKENKK | 0.98 | Sequence | 0.550695 | 1.3 | <= |
| | 1401 | | KENKKH | | | HNI | | | | | WB |
| | | | NIK | | | | | | | | |
| 137 | DRB1_ | 585 | ILVEDI | 5 | 1123 | IKENKK | 0.98 | Sequence | 0.550695 | 1.3 | <= |
| | 1454 | | KENKKH | | | HNI | | | | | WB |
| | | | NIK | | | | | | | | |
| 137 | DRB1_ | 585 | ILVEDI | 5 | 1123 | IKENKK | 1 | Sequence | 0.702691 | 0.11 | <= |
| | 1302 | | KENKKH | | | HNI | | | | | SB |
| | | | NIK | | | | | | | | |
| 138 | DRB1_ | 586 | LVEDIK | 4 | 1123 | IKENKK | 0.953 | Sequence | 0.347636 | 3.27 | <= |
| | 0301 | | ENKKHN | | | HNI | | | | | WB |
| | | | IKG | | | | | | | | |
| 138 | DRB1_ | 586 | LVEDIK | 4 | 1123 | IKENKK | 0.953 | Sequence | 0.14679 | 3.39 | <= |
| | 0305 | | ENKKHN | | | HNI | | | | | WB |
| | | | IKG | | | | | | | | |
| 138 | DRB1_ | 586 | LVEDIK | 4 | 1123 | IKENKK | 0.993 | Sequence | 0.449249 | 3.64 | <= |
| | 1101 | | ENKKHN | | | HNI | | | | | WB |
| | | | IKG | | | | | | | | |
| 138 | DRB1_ | 586 | LVEDIK | 4 | 1123 | IKENKK | 1 | Sequence | 0.685094 | 1.66 | <= |
| | 1104 | | ENKKHN | | | HNI | | | | | WB |
| | | | IKG | | | | | | | | |
| 138 | DRB1_ | 586 | LVEDIK | 4 | 1123 | IKENKK | 1 | Sequence | 0.863703 | 0.27 | <= |
| | 1301 | | ENKKHN | | | HNI | | | | | SB |
| | | | IKG | | | | | | | | |
| 138 | DRB1_ | 586 | LVEDIK | 4 | 1123 | IKENKK | 1 | Sequence | 0.603399 | 0.94 | <= |
| | 1401 | | ENKKHN | | | HNI | | | | | SB |
| | | | IKG | | | | | | | | |
| 138 | DRB1_ | 586 | LVEDIK | 4 | 1123 | IKENKK | 1 | Sequence | 0.603399 | 0.94 | <= |
| | 1454 | | ENKKHN | | | HNI | | | | | SB |
| | | | IKG | | | | | | | | |
| 138 | DRB1_ | 586 | LVEDIK | 4 | 1123 | IKENKK | 0.993 | Sequence | 0.228068 | 2.29 | <= |
| | 1402 | | ENKKHN | | | HNI | | | | | WB |
| | | | IKG | | | | | | | | |
| 138 | DRB1_ | 586 | LVEDIK | 4 | 1123 | IKENKK | 0.993 | Sequence | 0.373597 | 3.86 | <= |
| | 1303 | | ENKKHN | | | HNI | | | | | WB |
| | | | IKG | | | | | | | | |
| 138 | DRB1_ | 586 | LVEDIK | 4 | 1123 | IKENKK | 1 | Sequence | 0.768821 | 0.04 | <= |
| | 1302 | | ENKKHN | | | HNI | | | | | SB |
| | | | IKG | | | | | | | | |
| 139 | DRB1_ | 587 | VEDIKE | 3 | 1123 | IKENKK | 1 | Sequence | 0.415335 | 2.67 | <= |
| | 0301 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.19153 | 2.58 | <= |
| | 0305 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.529622 | 2.79 | <= |
| | 1101 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.742357 | 1.26 | <= |
| | 1104 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.290102 | 4.45 | <= |
| | 1201 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.892584 | 0.15 | <= |
| | 1301 | | NKKHNI | | | HNI | | | | | SB |
| | | | KGD | | | | | | | | |
| 139 | DRB1_ | 587 | VEDIKE | 3 | 1123 | IKENKK | 1 | Sequence | 0.657758 | 0.65 | <= |
| | 1401 | | NKKHNI | | | HNI | | | | | SB |
| | | | KGD | | | | | | | | |
| 139 | DRBI I454 | 587 | VEDIKE | 3 | 1123 | IKENKK | 1 | Sequence | 0.657758 | 0.65 | <= |
| | | | NKKHNI | | | HNI | | | | | SB |
| | | | KGD | | | | | | | | |
| 139 | DRB1_ | 587 | VEDIKE | 3 | 1123 | IKENKK | 1 | Sequence | 0.302256 | 1.09 | <= |
| | 1402 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.442925 | 3 | <= |
| | 1303 | | NKKHNI | | | HNI | | | | | WB |
| | | | KGD | | | | | | | | |
| 139 | DRB1_ | 587 | VEDIKE | 3 | 1123 | IKENKK | 1 | Sequence | 0.802181 | 0.02 | <= |
| | 1302 | | NKKHNI | | | HNI | | | | | SB |
| | | | KGD | | | | | | | | |
| 140 | DRB1_ | 588 | EDIKEN | 2 | 1123 | IKENKK | 1 | Sequence | 0.270476 | 4.2 | <= |
| | 0301 | | KKHNIK | | | HNI | | | | | WB |
| | | | GDR | | | | | | | | |
| 140 | DRB1_ | 588 | EDIKEN | 2 | 1123 | IKENKK | 0.987 | Sequence | 0.103126 | 4.7 | <= |
| | 0305 | | KKHNIK | | | HNI | | | | | WB |
| | | | GDR | | | | | | | | |
| 140 | DRB1_ | 588 | EDIKEN | 2 | 1123 | IKENKK | 0.98 | Sequence | 0.581086 | 2.55 | <= |
| | 1104 | | KKHNIK | | | HNI | | | | | WB |
| | | | GDR | | | | | | | | |
| 140 | DRB1_ | 588 | EDIKEN | 2 | 1123 | IKENKK | 1 | Sequence | 0.816513 | 0.51 | <= |
| | 1301 | | KKHNIK | | | HNI | | | | | SB |
| | | | GDR | | | | | | | | |
| 140 | DRB1_ | 588 | EDIKEN | 2 | 1123 | IKENKK | 1 | Sequence | 0.525771 | 1.51 | <= |
| | 1401 | | KKHNIK | | | HNI | | | | | WB |
| | | | GDR | | | | | | | | |
| 140 | DRB1_ | 588 | EDIKEN | 2 | 1123 | IKENKK | 1 | Sequence | 0.525771 | 1.51 | <= |
| | 1454 | | KKHNIK | | | HNI | | | | | WB |
| | | | GDR | | | | | | | | |
| 140 | DRB1_ | 588 | EDIKEN | 2 | 1123 | IKENKK | 0.993 | Sequence | 0.703569 | 0.11 | <= |
| | 1302 | | KKHNIK | | | HNI | | | | | SB |
| | | | GDR | | | | | | | | |
| 141 | DRB1_ | 589 | DIKENK | 1 | 1123 | IKENKK | 0.987 | Sequence | 0.524127 | 3.3 | <= |
| | 1301 | | KHNIKG | | | HNI | | | | | WB |
| | | | DRI | | | | | | | | |
| 141 | DRB1_ | 589 | DIKENK | 1 | 1123 | IKENKK | 0.98 | Sequence | 0.425941 | 1.86 | <= |
| | 1302 | | KHNIKG | | | HNI | | | | | WB |
| | | | DRI | | | | | | | | |
| 145 | DRB1_ | 590 | NKKHNI | 5 | 1124 | IKGDRI | 0.993 | Sequence | 0.464798 | 2.29 | <= |
| | 0301 | | KGDRIV | | | VLN | | | | | WB |
| | | | LNK | | | | | | | | |
| 145 | DRB1_ | 590 | NKKHNI | 5 | 1124 | IKGDRI | 0.98 | Sequence | 0.187355 | 2.65 | <= |
| | 0305 | | KGDRIV | | | VLN | | | | | WB |
| | | | LNK | | | | | | | | |
| 145 | DRB1_ | 590 | NKKHNI | 5 | 1124 | IKGDRI | 0.96 | Sequence | 0.16878 | 4.3 | <= |
| | 1402 | | KGDRIV | | | VLN | | | | | WB |
| | | | LNK | | | | | | | | |
| 146 | DRB1_ | 591 | KKHNIK | 4 | 1124 | IKGDRI | 1 | Sequence | 0.607216 | 1.43 | <= |
| | 0301 | | GDRIVL | | | VLN | | | | | WB |
| | | | NKN | | | | | | | | |
| 146 | DRB1_ | 591 | KKHNIK | 4 | 1124 | IKGDRI | 1 | Sequence | 0.272349 | 1.69 | <= |
| | 0305 | | GDRIVL | | | VLN | | | | | WB |
| | | | NKN | | | | | | | | |
| 146 | DRB1_ | 591 | KKHNIK | 4 | 1124 | IKGDRI | 1 | Sequence | 0.337273 | 3.9 | <= |
| | 0401 | | GDRIVL | | | VLN | | | | | WB |
| | | | NKN | | | | | | | | |
| 146 | DRB1_ | 591 | KKHNIK | 4 | 1124 | IKGDRI | 1 | Sequence | 0.425079 | 4.05 | <= |
| | 0403 | | GDRIVL | | | VLN | | | | | WB |
| | | | NKN | | | | | | | | |
| 146 | DRB1_ | 591 | KKHNIK | 4 | 1124 | IKGDRI | 0.98 | Sequence | 0.251433 | 1.77 | <= |
| | 1402 | | GDRIVL | | | VLN | | | | | WB |
| | | | NKN | | | | | | | | |
| 146 | DRB1_ | 591 | KKHNIK | 4 | 1124 | IKGDRI | 0.953 | Sequence | 0.331181 | 3.89 | <= |
| | 1302 | | GDRIVL | | | VLN | | | | | WB |
| | | | NKN | | | | | | | | |
| 147 | DRB1_ | 592 | KHNIKG | 3 | 1124 | IKGDRI | 1 | Sequence | 0.642187 | 1.25 | <= |
| | 0301 | | DRIVLN | | | VLN | | | | | WB |
| | | | KNS | | | | | | | | |
| 147 | DRB1_ | 592 | KHNIKG | 3 | 1124 | IKGDRI | 1 | Sequence | 0.303834 | 1.42 | <= |
| | 0305 | | DRIVLN | | | VLN | | | | | WB |
| | | | KNS | | | | | | | | |
| 147 | DRB1_ | 592 | KHNIKG | 3 | 1124 | IKGDRI | 1 | Sequence | 0.389574 | 3.16 | <= |
| | 0401 | | DRIVLN | | | VLN | | | | | WB |
| | | | KNS | | | | | | | | |
| 147 | DRB1_ | 592 | KHNIKG | 3 | 1124 | IKGDRI | 1 | Sequence | 0.466507 | 3.39 | <= |
| | 0403 | | DRIVLN | | | VLN | | | | | WB |
| | | | KNS | | | | | | | | |
| 147 | DRB1_ | 592 | KHNIKG | 3 | 1124 | IKGDRI | 0.987 | Sequence | 0.397232 | 4.69 | <= |
| | 1001 | | DRIVLN | | | VLN | | | | | WB |
| | | | KNS | | | | | | | | |
| 147 | DRB1_ | 592 | KHNIKG | 3 | 1124 | IKGDRI | 0.993 | Sequence | 0.282647 | 1.31 | <= |
| | 1402 | | DRIVLN | | | VLN | | | | | WB |
| | | | KNS | | | | | | | | |
| 147 | DRB1_ | 592 | KHNIKG | 3 | 1124 | IKGDRI | 1 | Sequence | 0.308928 | 4.91 | <= |
| | 1303 | | DRIVLN | | | VLN | | | | | WB |
| | | | KNS | | | | | | | | |
| 147 | DRB1_ | 592 | KHNIKG | 3 | 1124 | IKGDRI | 1 | Sequence | 0.375354 | 2.75 | <= |
| | 1302 | | DRIVLN | | | VLN | | | | | WB |
| | | | KNS | | | | | | | | |
| 148 | DRB1_ | 593 | HNIKGD | 2 | 1124 | IKGDRI | 1 | Sequence | 0.383408 | 2.95 | <= |
| | 0301 | | RIVLNK | | | VLN | | | | | WB |
| | | | NSK | | | | | | | | |
| 148 | DRB1_ | 593 | HNIKGD | 2 | 1124 | IKGDRI | 0.987 | Sequence | 0.138144 | 3.59 | <= |
| | 0305 | | RIVLNK | | | VLN | | | | | WB |
| | | | NSK | | | | | | | | |
| 149 | DRB1_ | 594 | NIKGDR | 6 | 1125 | IVLNKN | 0.913 | Sequence | 0.314003 | 4.4 | <= |
| | 1302 | | IVLNKN | | | SKK | | | | | WB |
| | | | SKK | | | | | | | | |
| 150 | DRB1_ | 595 | IKGDRI | 5 | 1125 | IVLNKN | 0.973 | Sequence | 0.295483 | 3.85 | <= |
| | 0301 | | VLNKNS | | | SKK | | | | | WB |
| | | | KKL | | | | | | | | |
| 150 | DRB1_ | 595 | IKGDRI | 5 | 1125 | IVLNKN | 0.687 | Sequence | 0.28066 | 4.34 | <= |
| | 0402 | | VLNKNS | | | SKK | | | | | WB |
| | | | KKL | | | | | | | | |
| 150 | DRB1_ | 595 | IKGDRI | 5 | 1125 | IVLNKN | 0.927 | Sequence | 0.521602 | 3.33 | <= |
| | 1301 | | VLNKNS | | | SKK | | | | | WB |
| | | | KKL | | | | | | | | |
| 150 | DRB1_ | 595 | IKGDRI | 5 | 1125 | IVLNKN | 0.927 | Sequence | 0.201172 | 3.05 | <= |
| | 1402 | | VLNKNS | | | SKK | | | | | WB |
| | | | KKL | | | | | | | | |
| 150 | DRB1_ | 595 | IKGDRI | 5 | 1125 | IVLNKN | 0.96 | Sequence | 0.499588 | 1.04 | <= |
| | 1302 | | VLNKNS | | | SKK | | | | | WB |
| | | | KKL | | | | | | | | |
| 151 | DRB1_ | 596 | KGDRIV | 4 | 1125 | IVLNKN | 0.94 | Sequence | 0.375778 | 3.01 | <= |
| | 0301 | | LNKNSK | | | SKK | | | | | WB |
| | | | KLY | | | | | | | | |
| 151 | DRB1_ | 596 | KGDRIV | 4 | 1125 | IVLNKN | 0.947 | Sequence | 0.132189 | 3.75 | <= |
| | 0305 | | LNKNSK | | | SKK | | | | | WB |
| | | | KLY | | | | | | | | |
| 151 | DRB1_ | 596 | KGDRIV | 4 | 1125 | IVLNKN | 0.76 | Sequence | 0.346271 | 3.18 | <= |
| | 0402 | | LNKNSK | | | SKK | | | | | WB |
| | | | KLY | | | | | | | | |
| 151 | DRB1_ | 596 | KGDRIV | 4 | 1125 | IVLNKN | 0.827 | Sequence | 0.650285 | 1.79 | <= |
| | 1301 | | LNKNSK | | | SKK | | | | | WB |
| | | | KLY | | | | | | | | |
| 151 | DRB1_ | 596 | KGDRIV | 4 | 1125 | IVLNKN | 0.967 | Sequence | 0.260038 | 1.64 | <= |
| | 1402 | | LNKNSK | | | SKK | | | | | WB |
| | | | KLY | | | | | | | | |
| 151 | DRB1_ | 596 | KGDRIV | 4 | 1125 | IVLNKN | 0.92 | Sequence | 0.582284 | 0.47 | <= |
| | 1302 | | LNKNSK | | | SKK | | | | | SB |
| | | | KLY | | | | | | | | |
| 152 | DRB1_ | 597 | GDRIVL | 4 | 1126 | VLNKNS | 0.867 | Sequence | 0.341749 | 3.15 | <= |
| | 0103 | | NKNSKK | | | KKL | | | | | WB |
| | | | LYA | | | | | | | | |
| 152 | DRB1_ | 597 | GDRIVL | 3 | 1125 | IVLNKN | 0.82 | Sequence | 0.454482 | 2.36 | <= |
| | 0301 | | NKNSKK | | | SKK | | | | | WB |
| | | | LYA | | | | | | | | |
| 152 | DRB1_ | 597 | GDRIVL | 3 | 1125 | IVLNKN | 0.887 | Sequence | 0.157728 | 3.15 | <= |
| | 0305 | | NKNSKK | | | SKK | | | | | WB |
| | | | LYA | | | | | | | | |
| 152 | DRB1_ | 597 | GDRIVL | 3 | 1125 | IVLNKN | 0.833 | Sequence | 0.39964 | 2.48 | <= |
| | 0402 | | NKNSKK | | | SKK | | | | | WB |
| | | | LYA | | | | | | | | |
| 152 | DRBI I104 | 597 | GDRIVL | 3 | 1125 | IVLNKN | 0.78 | Sequence | 0.392042 | 4.97 | <= |
| | | | NKNSKK | | | SKK | | | | | WB |
| | | | LYA | | | | | | | | |
| 152 | DRB1_ | 597 | GDRIVL | 3 | 1125 | IVLNKN | 0.64 | Sequence | 0.755508 | 0.9 | <= |
| | 1301 | | NKNSKK | | | SKK | | | | | SB |
| | | | LYA | | | | | | | | |
| 152 | DRB1_ | 597 | GDRIVL | 3 | 1125 | IVLNKN | 0.587 | Sequence | 0.308016 | 4.92 | <= |
| | 1401 | | NKNSKK | | | SKK | | | | | WB |
| | | | LYA | | | | | | | | |
| 152 | DRB1_ | 597 | GDRIVL | 4 | 1126 | VLNKNS | 0.573 | Sequence | 0.210974 | 4.75 | <= |
| | 1501 | | NKNSKK | | | KKL | | | | | WB |
| | | | LYA | | | | | | | | |
| 152 | DRB1_ | 597 | GDRIVL | 4 | 1126 | VLNKNS | 0.547 | Sequence | 0.192524 | 4.31 | <= |
| | 1503 | | NKNSKK | | | KKL | | | | | WB |
| | | | LYA | | | | | | | | |
| 152 | DRB1_ | 597 | GDRIVL | 3 | 1125 | IVLNKN | 0.587 | Sequence | 0.308016 | 4.92 | <= |
| | 1454 | | NKNSKK | | | SKK | | | | | WB |
| | | | LYA | | | | | | | | |
| 152 | DRB1_ | 597 | GDRIVL | 3 | 1125 | IVLNKN | 0.927 | Sequence | 0.298161 | 1.14 | <= |
| | 1402 | | NKNSKK | | | SKK | | | | | WB |
| | | | LYA | | | | | | | | |
| 152 | DRB1_ | 597 | GDRIVL | 3 | 1125 | IVLNKN | 0.773 | Sequence | 0.644196 | 0.23 | <= |
| | 1302 | | NKNSKK | | | SKK | | | | | SB |
| | | | LYA | | | | | | | | |
| 153 | DRB1_ | 598 | DRIVLN | 3 | 1126 | VLNKNS | 0.947 | Sequence | 0.3229 | 3.43 | <= |
| | 0103 | | KNSKKL | | | KKL | | | | | WB |
| | | | YAI | | | | | | | | |
| 153 | DRB1_ | 598 | DRIVLN | 2 | 1125 | IVLNKN | 0.533 | Sequence | 0.3326 | 3.41 | <= |
| | 0301 | | KNSKKL | | | SKK | | | | | WB |
| | | | YAI | | | | | | | | |
| 153 | DRB1_ | 598 | DRIVLN | 4 | 1127 | LNKNSK | 0.493 | Sequence | 0.688503 | 1.41 | <= |
| | 1301 | | KNSKKL | | | KLY | | | | | WB |
| | | | YAI | | | | | | | | |
| 153 | DRB1_ | 598 | DRIVLN | 2 | 1125 | IVLNKN | 0.733 | Sequence | 0.166805 | 4.39 | <= |
| | 1402 | | KNSKKL | | | SKK | | | | | WB |
| | | | YAI | | | | | | | | |
| 153 | DRB1_ | 598 | DRIVLN | 2 | 1125 | IVLNKN | 0.527 | Sequence | 0.534139 | 0.75 | <= |
| | 1302 | | KNSKKL | | | SKK | | | | | SB |
| | | | YAI | | | | | | | | |
| 154 | DRB1_ | 599 | RIVLNK | 3 | 1127 | LNKNSK | 0.82 | Sequence | 0.271781 | 4.18 | <= |
| | 0301 | | NSKKLY | | | KLY | | | | | WB |
| | | | AIG | | | | | | | | |
| 154 | DRB1_ | 599 | RIVLNK | 3 | 1127 | LNKNSK | 0.847 | Sequence | 0.659488 | 1.68 | <= |
| | 1301 | | NSKKLY | | | KLY | | | | | WB |
| | | | AIG | | | | | | | | |
| 154 | DRB1_ | 599 | RIVLNK | 3 | 1127 | LNKNSK | 0.753 | Sequence | 0.459394 | 1.46 | <= |
| | 1302 | | NSKKLY | | | KLY | | | | | WB |
| | | | AIG | | | | | | | | |
| 158 | DRB1_ | 600 | NKNSKK | 6 | 1128 | LYAIGN | 1 | Sequence | 0.400658 | 2.37 | <= |
| | 1201 | | LYAIGN | | | VEY | | | | | WB |
| | | | VEY | | | | | | | | |
| 159 | DRB1_ | 601 | KNSKKL | 5 | 1128 | LYAIGN | 1 | Sequence | 0.681655 | 0.45 | <= |
| | 1201 | | YAIGNV | | | VEY | | | | | SB |
| | | | EYI | | | | | | | | |
| 160 | DRB1_ | 602 | NSKKLY | 5 | 1129 | YAIGNV | 0.9 | Sequence | 0.46205 | 2.78 | <= |
| | 0901 | | AIGNVE | | | EYI | | | | | WB |
| | | | YIL | | | | | | | | |
| 160 | DRB1_ | 602 | NSKKLY | 4 | 1128 | LYAIGN | 1 | Sequence | 0.69546 | 0.41 | <= |
| | 1201 | | AIGNVE | | | VEY | | | | | SB |
| | | | YIL | | | | | | | | |
| 161 | DRB1_ | 603 | SKKLYA | 3 | 1128 | LYAIGN | 0.96 | Sequence | 0.3438 | 4.69 | <= |
| | 0404 | | IGNVEY | | | VEY | | | | | WB |
| | | | ILD | | | | | | | | |
| 161 | DRB1_ | 603 | SKKLYA | 4 | 1129 | YAIGNV | 0.707 | Sequence | 0.281269 | 4.19 | <= |
| | 0701 | | IGNVEY | | | EYI | | | | | WB |
| | | | ILD | | | | | | | | |
| 161 | DRB1_ | 603 | SKKLYA | 4 | 1129 | YAIGNV | 0.94 | Sequence | 0.62682 | 1.04 | <= |
| | 0901 | | IGNVEY | | | EYI | | | | | WB |
| | | | ILD | | | | | | | | |
| 161 | DRB1_ | 603 | SKKLYA | 3 | 1128 | LYAIGN | 1 | Sequence | 0.777528 | 0.2 | <= |
| | 1201 | | IGNVEY | | | VEY | | | | | SB |
| | | | ILD | | | | | | | | |
| 161 | DRB1_ | 603 | SKKLYA | 3 | 1128 | LYAIGN | 0.84 | Sequence | 0.364687 | 3.6 | <= |
| | 1401 | | IGNVEY | | | VEY | | | | | WB |
| | | | ILD | | | | | | | | |
| 161 | DRB1_ | 603 | SKKLYA | 3 | 1128 | LYAIGN | 0.973 | Sequence | 0.199557 | 4.11 | <= |
| | 1503 | | IGNVEY | | | VEY | | | | | WB |
| | | | ILD | | | | | | | | |
| 161 | DRB1_ | 603 | SKKLYA | 3 | 1128 | LYAIGN | 0.84 | Sequence | 0.364687 | 3.6 | <= |
| | 1454 | | IGNVEY | | | VEY | | | | | WB |
| | | | ILD | | | | | | | | |
| 161 | DRB1_ | 603 | SKKLYA | 3 | 1128 | LYAIGN | 0.72 | Sequence | 0.16351 | 4.55 | <= |
| | 1402 | | IGNVEY | | | VEY | | | | | WB |
| | | | ILD | | | | | | | | |
| 161 | DRB1_ | 603 | SKKLYA | 3 | 1128 | LYAIGN | 0.787 | Sequence | 0.326924 | 4.02 | <= |
| | 1302 | | IGNVEY | | | VEY | | | | | WB |
| | | | ILD | | | | | | | | |
| 162 | DRB1_ | 604 | KKLYAI | 3 | 1129 | YAIGNV | 0.987 | Sequence | 0.585673 | 1.33 | <= |
| | 0901 | | GNVEYI | | | EYI | | | | | WB |
| | | | LDM | | | | | | | | |
| 162 | DRB1_ | 604 | KKLYAI | 2 | 1128 | LYAIGN | 0.993 | Sequence | 0.603606 | 0.76 | <= |
| | 1201 | | GNVEYI | | | VEY | | | | | SB |
| | | | LDM | | | | | | | | |
| 167 | DRB1_ | 605 | IGNVEY | 5 | 1130 | YILDMD | 0.94 | Sequence | 0.178042 | 2.79 | <= |
| | 0305 | | ILDMDT | | | TNE | | | | | WB |
| | | | NEK | | | | | | | | |
| 167 | DRB1_ | 605 | IGNVEY | 5 | 1130 | YILDMD | 0.967 | Sequence | 0.534443 | 1.84 | <= |
| | 0401 | | ILDMDT | | | TNE | | | | | WB |
| | | | NEK | | | | | | | | |
| 167 | DRB1_ | 605 | IGNVEY | 5 | 1130 | YILDMD | 0.753 | Sequence | 0.532785 | 2.52 | <= |
| | 0403 | | ILDMDT | | | TNE | | | | | WB |
| | | | NEK | | | | | | | | |
| 167 | DRB1_ | 605 | IGNVEY | 3 | 1131 | VEYILD | 0.88 | Sequence | 0.350447 | 4.58 | <= |
| | 0404 | | ILDMDT | | | MDT | | | | | WB |
| | | | NEK | | | | | | | | |
| 167 | DRB1_ | 605 | IGNVEY | 5 | 1130 | YILDMD | 0.987 | Sequence | 0.638733 | 1.1 | <= |
| | 0405 | | ILDMDT | | | TNE | | | | | WB |
| | | | NEK | | | | | | | | |
| 167 | DRB1_ | 605 | IGNVEY | 5 | 1130 | YILDMD | 0.967 | Sequence | 0.346308 | 4.27 | <= |
| | 1303 | | ILDMDT | | | TNE | | | | | WB |
| | | | NEK | | | | | | | | |
| 167 | DRB1_ | 605 | IGNVEY | 5 | 1130 | YILDMD | 0.773 | Sequence | 0.400391 | 3.68 | <= |
| | 0408 | | ILDMDT | | | TNE | | | | | WB |
| | | | NEK | | | | | | | | |
| 168 | DRB1_ | 606 | GNVEYI | 4 | 1130 | YILDMD | 0.767 | Sequence | 0.215403 | 2.29 | <= |
| | 0305 | | LDMDTN | | | TNE | | | | | WB |
| | | | EKL | | | | | | | | |
| 168 | DRB1_ | 606 | GNVEYI | 4 | 1130 | YILDMD | 0.96 | Sequence | 0.59063 | 1.43 | <= |
| | 0401 | | LDMDTN | | | TNE | | | | | WB |
| | | | EKL | | | | | | | | |
| 168 | DRB1_ | 606 | GNVEYI | 4 | 1130 | YILDMD | 0.847 | Sequence | 0.540959 | 2.43 | <= |
| | 0403 | | LDMDTN | | | TNE | | | | | WB |
| | | | EKL | | | | | | | | |
| 168 | DRB1_ | 606 | GNVEYI | 4 | 1130 | YILDMD | 1 | Sequence | 0.662819 | 1 | <= |
| | 0405 | | LDMDTN | | | TNE | | | | | SB |
| | | | EKL | | | | | | | | |
| 168 | DRB1_ | 606 | GNVEYI | 4 | 1130 | YILDMD | 0.953 | Sequence | 0.378261 | 3.8 | <= |
| | 1303 | | LDMDTN | | | TNE | | | | | WB |
| | | | EKL | | | | | | | | |
| 168 | DRB1_ | 606 | GNVEYI | 4 | 1130 | YILDMD | 0.907 | Sequence | 0.44686 | 3.11 | <= |
| | 0408 | | LDMDTN | | | TNE | | | | | WB |
| | | | EKL | | | | | | | | |
| 169 | DRB1_ | 607 | NVEYIL | 5 | 1132 | LDMDTN | 0.867 | Sequence | 0.443181 | 2.45 | <= |
| | 0301 | | DMDTNE | | | EKL | | | | | WB |
| | | | KLY | | | | | | | | |
| 169 | DRB1_ | 607 | NVEYIL | 3 | 1130 | YILDMD | 0.533 | Sequence | 0.306998 | 1.39 | <= |
| | 0305 | | DMDTNE | | | TNE | | | | | WB |
| | | | KLY | | | | | | | | |
| 169 | DRB1_ | 607 | NVEYIL | 3 | 1130 | YILDMD | 0.913 | Sequence | 0.638387 | 1.14 | <= |
| | 0401 | | DMDTNE | | | TNE | | | | | WB |
| | | | KLY | | | | | | | | |
| 169 | DRB1_ | 607 | NVEYIL | 3 | 1130 | YILDMD | 0.8 | Sequence | 0.578826 | 2.04 | <= |
| | 0403 | | DMDTNE | | | TNE | | | | | WB |
| | | | KLY | | | | | | | | |
| 169 | DRB1_ | 607 | NVEYIL | 4 | 1133 | ILDMDT | 0.62 | Sequence | 0.329329 | 4.95 | <= |
| | 0404 | | DMDTNE | | | NEK | | | | | WB |
| | | | KLY | | | | | | | | |
| 169 | DRB1_ | 607 | NVEYIL | 3 | 1130 | YILDMD | 0.987 | Sequence | 0.685826 | 0.91 | <= |
| | 0405 | | DMDTNE | | | TNE | | | | | SB |
| | | | KLY | | | | | | | | |
| 169 | DRB1_ | 607 | NVEYIL | 3 | 1130 | YILDMD | 0.933 | Sequence | 0.406743 | 3.42 | <= |
| | 1303 | | DMDTNE | | | TNE | | | | | WB |
| | | | KLY | | | | | | | | |
| 169 | DRB1_ | 607 | NVEYIL | 3 | 1130 | YILDMD | 0.867 | Sequence | 0.502225 | 2.54 | <= |
| | 0408 | | DMDTNE | | | TNE | | | | | WB |
| | | | KLY | | | | | | | | |
| 170 | DRB1_ | 608 | VEYILD | 4 | 1132 | LDMDTN | 0.94 | Sequence | 0.50644 | 1.99 | <= |
| | 0301 | | MDTNEK | | | EKL | | | | | WB |
| | | | LYF | | | | | | | | |
| 170 | DRB1_ | 608 | VEYILD | 4 | 1132 | LDMDTN | 0.76 | Sequence | 0.265171 | 1.76 | <= |
| | 0305 | | MDTNEK | | | EKL | | | | | WB |
| | | | LYF | | | | | | | | |
| 170 | DRB1_ | 608 | VEYILD | 2 | 1130 | YILDMD | 0.647 | Sequence | 0.360298 | 3.54 | <= |
| | 0401 | | MDTNEK | | | TNE | | | | | WB |
| | | | LYF | | | | | | | | |
| 170 | DRB1_ | 608 | VEYILD | 2 | 1130 | YILDMD | 0.873 | Sequence | 0.36958 | 3 | <= |
| | 0405 | | MDTNEK | | | TNE | | | | | WB |
| | | | LYF | | | | | | | | |
| 171 | DRB1_ | 609 | EYILDM | 3 | 1132 | LDMDTN | 0.927 | Sequence | 0.541685 | 1.78 | <= |
| | 0301 | | DTNEKL | | | EKL | | | | | WB |
| | | | YFY | | | | | | | | |
| 171 | DRB1_ | 609 | EYILDM | 3 | 1132 | LDMDTN | 0.913 | Sequence | 0.252904 | 1.88 | <= |
| | 0305 | | DTNEKL | | | EKL | | | | | WB |
| | | | YFY | | | | | | | | |
| 172 | DRB1_ | 610 | YILDMD | 2 | 1132 | LDMDTN | 0.633 | Sequence | 0.324851 | 3.48 | <= |
| | 0301 | | TNEKLY | | | EKL | | | | | WB |
| | | | FYG | | | | | | | | |
| 172 | DRB1_ | 610 | YILDMD | 2 | 1132 | LDMDTN | 0.713 | Sequence | 0.119618 | 4.11 | <= |
| | 0305 | | TNEKLY | | | EKL | | | | | WB |
| | | | FYG | | | | | | | | |
| 173 | DRB1_ | 611 | ILDMDT | 3 | 1134 | MDTNEK | 0.753 | Sequence | 0.259419 | 4.37 | <= |
| | 0301 | | NEKLYF | | | LYF | | | | | WB |
| | | | YGN | | | | | | | | |
| 176 | DRB1_ | 612 | MDTNEK | 6 | 1135 | LYFYGN | 0.987 | Sequence | 0.344701 | 2.83 | <= |
| | 1501 | | LYFYGN | | | EFL | | | | | WB |
| | | | EFL | | | | | | | | |
| 176 | DRB1_ | 612 | MDTNEK | 6 | 1135 | LYFYGN | 0.96 | Sequence | 0.210071 | 3.87 | <= |
| | 1503 | | LYFYGN | | | EFL | | | | | WB |
| | | | EFL | | | | | | | | |
| 177 | DRB1_ | 613 | DTNEKL | 5 | 1135 | LYFYGN | 1 | Sequence | 0.785598 | 0.56 | <= |
| | 1501 | | YFYGNE | | | EFL | | | | | SB |
| | | | FLV | | | | | | | | |
| 177 | DRB1_ | 613 | DTNEKL | 5 | 1135 | LYFYGN | 1 | Sequence | 0.473471 | 1.09 | <= |
| | 1503 | | YFYGNE | | | EFL | | | | | WB |
| | | | FLV | | | | | | | | |
| 178 | DRB1_ | 614 | TNEKLY | 5 | 1136 | YFYGNE | 0.94 | Sequence | 0.463835 | 2.75 | <= |
| | 0901 | | FYGNEF | | | FLV | | | | | WB |
| | | | LVD | | | | | | | | |
| 178 | DRB1_ | 614 | TNEKLY | 4 | 1135 | LYFYGN | 1 | Sequence | 0.920165 | 0.19 | <= |
| | 1501 | | FYGNEF | | | EFL | | | | | SB |
| | | | LVD | | | | | | | | |
| 178 | DRB1_ | 614 | TNEKLY | 4 | 1135 | LYFYGN | 0.92 | Sequence | 0.438268 | 3.24 | <= |
| | 1601 | | FYGNEF | | | EFL | | | | | WB |
| | | | LVD | | | | | | | | |
| 178 | DRB1_ | 614 | TNEKLY | 4 | 1135 | LYFYGN | 0.993 | Sequence | 0.690426 | 0.35 | <= |
| | 1503 | | FYGNEF | | | EFL | | | | | SB |
| | | | LVD | | | | | | | | |
| 179 | DRB1_ | 615 | NEKLYF | 4 | 1136 | YFYGNE | 0.973 | Sequence | 0.484479 | 2.44 | <= |
| | 0901 | | YGNEFL | | | FLV | | | | | WB |
| | | | VDF | | | | | | | | |
| 179 | DRB1_ | 615 | NEKLYF | 3 | 1135 | LYFYGN | 1 | Sequence | 0.901266 | 0.24 | <= |
| | 1501 | | YGNEFL | | | EFL | | | | | SB |
| | | | VDF | | | | | | | | |
| 179 | DRB1_ | 615 | NEKLYF | 3 | 1135 | LYFYGN | 0.853 | Sequence | 0.40851 | 3.62 | <= |
| | 1601 | | YGNEFL | | | EFL | | | | | WB |
| | | | VDF | | | | | | | | |
| 179 | DRB1_ | 615 | NEKLYF | 3 | 1135 | LYFYGN | 0.993 | Sequence | 0.647087 | 0.45 | <= |
| | 1503 | | YGNEFL | | | EFL | | | | | SB |
| | | | VDF | | | | | | | | |
| 180 | DRB1_ | 616 | EKLYFY | 3 | 1136 | YFYGNE | 0.987 | Sequence | 0.595289 | 1.26 | <= |
| | 0901 | | GNEFLV | | | FLV | | | | | WB |
| | | | DFD | | | | | | | | |
| 180 | DRB1_ | 616 | EKLYFY | 2 | 1135 | LYFYGN | 1 | Sequence | 0.712864 | 0.77 | <= |
| | 1501 | | GNEFLV | | | EFL | | | | | SB |
| | | | DFD | | | | | | | | |
| 180 | DRB1_ | 616 | EKLYFY | 2 | 1135 | LYFYGN | 0.92 | Sequence | 0.391802 | 1.57 | <= |
| | 1503 | | GNEFLV | | | EFL | | | | | WB |
| | | | DFD | | | | | | | | |
| 181 | DRB1_ | 617 | KLYFYG | 1 | 1135 | LYFYGN | 0.973 | Sequence | 0.212741 | 4.72 | <= |
| | 1501 | | NEFLVD | | | EFL | | | | | WB |
| | | | FDS | | | | | | | | |
| 184 | DRB1_ | 618 | FYGNEF | 5 | 1137 | FLVDFD | 0.94 | Sequence | 0.103324 | 4.7 | <= |
| | 0305 | | LVDFDS | | | SQN | | | | | WB |
| | | | QNF | | | | | | | | |
| 184 | DRB1_ | 618 | FYGNEF | 5 | 1137 | FLVDFD | 1 | Sequence | 0.496435 | 2.13 | <= |
| | 0401 | | LVDFDS | | | SQN | | | | | WB |
| | | | QNF | | | | | | | | |
| 184 | DRB1_ | 618 | FYGNEF | 5 | 1137 | FLVDFD | 0.973 | Sequence | 0.306359 | 3.87 | <= |
| | 0405 | | LVDFDS | | | SQN | | | | | WB |
| | | | QNF | | | | | | | | |
| 184 | DRB1_ | 618 | FYGNEF | 5 | 1137 | FLVDFD | 0.993 | Sequence | 0.319794 | 4.99 | <= |
| | 0408 | | LVDFDS | | | SQN | | | | | WB |
| | | | QNF | | | | | | | | |
| 185 | DRB1_ | 619 | YGNEFL | 4 | 1137 | FLVDFD | 0.9 | Sequence | 0.125158 | 3.94 | <= |
| | 0305 | | VDFDSQ | | | SQN | | | | | WB |
| | | | NFL | | | | | | | | |
| 185 | DRB1_ | 619 | YGNEFL | 4 | 1137 | FLVDFD | 0.993 | Sequence | 0.559449 | 1.65 | <= |
| | 0401 | | VDFDSQ | | | SQN | | | | | WB |
| | | | NFL | | | | | | | | |
| 185 | DRB1_ | 619 | YGNEFL | 4 | 1137 | FLVDFD | 0.973 | Sequence | 0.320964 | 3.64 | <= |
| | 0405 | | VDFDSQ | | | SQN | | | | | WB |
| | | | NFL | | | | | | | | |
| 185 | DRB1_ | 619 | YGNEFL | 4 | 1137 | FLVDFD | 0.993 | Sequence | 0.374345 | 4.06 | <= |
| | 0408 | | VDFDSQ | | | SQN | | | | | WB |
| | | | NFL | | | | | | | | |
| 186 | DRB1_ | 620 | GNEFLV | 3 | 1137 | FLVDFD | 0.847 | Sequence | 0.106568 | 4.56 | <= |
| | 0305 | | DFDSQN | | | SQN | | | | | WB |
| | | | FLL | | | | | | | | |
| 186 | DRB1_ | 620 | GNEFLV | 3 | 1137 | FLVDFD | 0.993 | Sequence | 0.480761 | 2.27 | <= |
| | 0401 | | DFDSQN | | | SQN | | | | | WB |
| | | | FLL | | | | | | | | |
| 186 | DRB1_ | 620 | GNEFLV | 3 | 1137 | FLVDFD | 0.94 | Sequence | 0.256902 | 4.78 | <= |
| | 0405 | | DFDSQN | | | SQN | | | | | WB |
| | | | FLL | | | | | | | | |
| 187 | DRB1_ | 621 | NEFLVD | 3 | 1138 | LVDFDS | 1 | Sequence | 0.26087 | 3.82 | <= |
| | 1501 | | FDSQNF | | | QNF | | | | | WB |
| | | | LLK | | | | | | | | |
| 187 | DRB1_ | 621 | NEFLVD | 3 | 1138 | LVDFDS | 0.987 | Sequence | 0.200252 | 4.1 | <= |
| | 1503 | | FDSQNF | | | QNF | | | | | WB |
| | | | LLK | | | | | | | | |
| 188 | DRB1_ | 622 | EFLVDF | 3 | 1139 | VDFDSQ | 0.987 | Sequence | 0.250303 | 4.51 | <= |
| | 0301 | | DSQNFL | | | NFL | | | | | WB |
| | | | LKN | | | | | | | | |
| 193 | DRB1_ | 623 | FDSQNF | 5 | 1140 | FLLKNG | 0.967 | Sequence | 0.359736 | 2.91 | <= |
| | 0103 | | LLKNGI | | | ILQ | | | | | WB |
| | | | LQK | | | | | | | | |
| 194 | DRB1_ | 624 | DSQNFL | 4 | 1140 | FLLKNG | 0.987 | Sequence | 0.628176 | 0.88 | <= |
| | 0103 | | LKNGIL | | | ILQ | | | | | SB |
| | | | QKK | | | | | | | | |
| 194 | DRB1_ | 624 | DSQNFL | 4 | 1140 | FLLKNG | 0.973 | Sequence | 0.469602 | 4.07 | <= |
| | 0801 | | LKNGIL | | | ILQ | | | | | WB |
| | | | QKK | | | | | | | | |
| 194 | DRB1_ | 624 | DSQNFL | 4 | 1140 | FLLKNG | 1 | Sequence | 0.497634 | 3.09 | <= |
| | 1101 | | LKNGIL | | | ILQ | | | | | WB |
| | | | QKK | | | | | | | | |
| 194 | DRB1_ | 624 | DSQNFL | 4 | 1140 | FLLKNG | 0.913 | Sequence | 0.34711 | 4.52 | <= |
| | 1601 | | LKNGIL | | | ILQ | | | | | WB |
| | | | QKK | | | | | | | | |
| 194 | DRB1_ | 624 | DSQNFL | 4 | 1140 | FLLKNG | 0.92 | Sequence | 0.323311 | 3.8 | <= |
| | 0803 | | LKNGIL | | | ILQ | | | | | WB |
| | | | QKK | | | | | | | | |
| 195 | DRB1_ | 625 | SQNFLL | 3 | 1140 | FLLKNG | 0.993 | Sequence | 0.613774 | 0.94 | <= |
| | 0103 | | KNGILQ | | | ILQ | | | | | SB |
| | | | KKM | | | | | | | | |
| 195 | DRB1_ | 625 | SQNFLL | 3 | 1140 | FLLKNG | 0.98 | Sequence | 0.430023 | 4.86 | <= |
| | 0801 | | KNGILQ | | | IL0 | | | | | WB |
| | | | KKM | | | | | | | | |
| 195 | DRB1_ | 625 | SQNFLL | 3 | 1140 | FLLKNG | 0.993 | Sequence | 0.465731 | 3.45 | <= |
| | 1101 | | KNGILQ | | | ILQ | | | | | WB |
| | | | KKM | | | | | | | | |
| 195 | DRB1_ | 625 | SQNFLL | 3 | 1140 | FLLKNG | 0.94 | Sequence | 0.295272 | 4.63 | <= |
| | 0803 | | KNGILQ | | | IL0 | | | | | WB |
| | | | KKM | | | | | | | | |
| 196 | DRB1_ | 626 | QNFLLK | 2 | 1140 | FLLKNG | 0.987 | Sequence | 0.343837 | 3.12 | <= |
| | 0103 | | NGILQK | | | ILQ | | | | | WB |
| | | | KMQ | | | | | | | | |
| 209 | DRB1_ | 627 | MQKNQI | 5 | 1141 | IDHILS | 0.98 | Sequence | 0.299912 | 3.94 | <= |
| | 0402 | | DHILSF | | | FGG | | | | | WB |
| | | | GGK | | | | | | | | |
| 209 | DRB1_ | 627 | MQKNQI | 5 | 1141 | IDHILS | 0.993 | Sequence | 0.383284 | 4.09 | <= |
| | 0404 | | DHILSF | | | FGG | | | | | WB |
| | | | GGK | | | | | | | | |
| 209 | DRB1_ | 627 | MQKNQI | 5 | 1141 | IDHILS | 1 | Sequence | 0.257628 | 3.87 | <= |
| | 1501 | | DHILSF | | | FGG | | | | | WB |
| | | | GGK | | | | | | | | |
| 210 | DRB1_ | 628 | QKNQID | 4 | 1141 | IDHILS | 0.987 | Sequence | 0.410574 | 2.37 | <= |
| | 0402 | | HILSFG | | | FGG | | | | | WB |
| | | | GKV | | | | | | | | |
| 210 | DRB1_ | 628 | QKNQID | 4 | 1141 | IDHILS | 0.993 | Sequence | 0.519887 | 2.5 | <= |
| | 0404 | | HILSFG | | | FGG | | | | | WB |
| | | | GKV | | | | | | | | |
| 210 | DRB1_ | 628 | QKNQID | 4 | 1141 | IDHILS | 1 | Sequence | 0.403208 | 2.33 | <= |
| | 1501 | | HILSFG | | | FGG | | | | | WB |
| | | | GKV | | | | | | | | |
| 210 | DRB1_ | 628 | QKNQID | 4 | 1141 | IDHILS | 1 | Sequence | 0.420323 | 3.46 | <= |
| | 1601 | | HILSFG | | | FGG | | | | | WB |
| | | | GKV | | | | | | | | |
| 210 | DRB1_ | 628 | QKNQID | 4 | 1141 | IDHILS | 0.993 | Sequence | 0.263305 | 2.9 | <= |
| | 1503 | | HILSFG | | | FGG | | | | | WB |
| | | | GKV | | | | | | | | |
| 211 | DRB1_ | 629 | KNQIDH | 3 | 1141 | IDHILS | 0.987 | Sequence | 0.331756 | 3.4 | <= |
| | 0402 | | ILSFGG | | | FGG | | | | | WB |
| | | | KVL | | | | | | | | |
| 211 | DRB1_ | 629 | KNQIDH | 3 | 1141 | IDHILS | 0.993 | Sequence | 0.442985 | 3.31 | <= |
| | 0404 | | ILSFGG | | | FGG | | | | | WB |
| | | | KVL | | | | | | | | |
| 211 | DRB1_ | 629 | KNQIDH | 3 | 1141 | IDHILS | 0.633 | Sequence | 0.429141 | 2.13 | <= |
| | 1501 | | ILSFGG | | | FGG | | | | | WB |
| | | | KVL | | | | | | | | |
| 211 | DRB1_ | 629 | KNQIDH | 3 | 1141 | IDHILS | 0.833 | Sequence | 0.376422 | 4.05 | <= |
| | 1601 | | ILSFGG | | | FGG | | | | | WB |
| | | | KVL | | | | | | | | |
| 211 | DRB1_ | 629 | KNQIDH | 3 | 1141 | IDHILS | 0.453 | Sequence | 0.315726 | 2.24 | <= |
| | 1503 | | ILSFGG | | | FGG | | | | | WB |
| | | | KVL | | | | | | | | |
| 212 | DRB1_ | 630 | NQIDHI | 5 | 1142 | ILSFGG | 0.967 | Sequence | 0.337205 | 3.22 | <= |
| | 0103 | | LSFGGK | | | KVL | | | | | WB |
| | | | VLK | | | | | | | | |
| 212 | DRB1_ | 630 | NQIDHI | 5 | 1142 | ILSFGG | 1 | Sequence | 0.839827 | 0.41 | <= |
| | 1501 | | LSFGGK | | | KVL | | | | | SB |
| | | | VLK | | | | | | | | |
| 212 | DRB1_ | 630 | NQIDHI | 5 | 1142 | ILSFGG | 0.973 | Sequence | 0.651711 | 1.23 | <= |
| | 1601 | | LSFGGK | | | KVL | | | | | WB |
| | | | VLK | | | | | | | | |
| 212 | DRB1_ | 630 | NQIDHI | 5 | 1142 | ILSFGG | 0.993 | Sequence | 0.698594 | 0.34 | <= |
| | 1503 | | LSFGGK | | | KVL | | | | | SB |
| | | | VLK | | | | | | | | |
| 213 | DRB1_ | 631 | QIDHIL | 4 | 1142 | ILSFGG | 0.98 | Sequence | 0.312299 | 4.35 | <= |
| | 0101 | | SFGGKV | | | KVL | | | | | WB |
| | | | LKK | | | | | | | | |
| 213 | DRB1_ | 631 | QIDHIL | 4 | 1142 | ILSFGG | 1 | Sequence | 0.601057 | 2.17 | <= |
| | 0102 | | SFGGKV | | | KVL | | | | | WB |
| | | | LKK | | | | | | | | |
| 213 | DRB1_ | 631 | QIDHIL | 4 | 1142 | ILSFGG | 0.993 | Sequence | 0.621389 | 0.91 | <= |
| | 0103 | | SFGGKV | | | KVL | | | | | SB |
| | | | LKK | | | | | | | | |
| 213 | DRB1_ | 631 | QIDHIL | 4 | 1142 | ILSFGG | 0.933 | Sequence | 0.29894 | 4.23 | <= |
| | 1201 | | SFGGKV | | | KVL | | | | | WB |
| | | | LKK | | | | | | | | |
| 213 | DRB1_ | 631 | QIDHIL | 4 | 1142 | ILSFGG | 1 | Sequence | 0.933948 | 0.16 | <= |
| | 1501 | | SFGGKV | | | KVL | | | | | SB |
| | | | LKK | | | | | | | | |
| 213 | DRB1_ | 631 | QIDHIL | 4 | 1142 | ILSFGG | 1 | Sequence | 0.832274 | 0.33 | <= |
| | 1601 | | SFGGKV | | | KVL | | | | | SB |
| | | | LKK | | | | | | | | |
| 213 | DRB1_ | 631 | QIDHIL | 4 | 1142 | ILSFGG | 1 | Sequence | 0.834253 | 0.11 | <= |
| | 1503 | | SFGGKV | | | KVL | | | | | SB |
| | | | LKK | | | | | | | | |
| 214 | DRB1_ | 632 | IDHILS | 3 | 1142 | ILSFGG | 0.987 | Sequence | 0.308163 | 4.4 | <= |
| | 0101 | | FGGKVL | | | KVL | | | | | WB |
| | | | KKI | | | | | | | | |
| 214 | DRB1_ | 632 | IDHILS | 3 | 1142 | ILSFGG | 1 | Sequence | 0.594855 | 2.21 | <= |
| | 0102 | | FGGKVL | | | KVL | | | | | WB |
| | | | KKI | | | | | | | | |
| 214 | DRB1_ | 632 | IDHILS | 3 | 1142 | ILSFGG | 0.993 | Sequence | 0.610336 | 0.96 | <= |
| | 0103 | | FGGKVL | | | KVL | | | | | SB |
| | | | KKI | | | | | | | | |
| 214 | DRB1_ | 632 | IDHILS | 3 | 1142 | ILSFGG | 0.967 | Sequence | 0.292311 | 4.39 | <= |
| | 1201 | | FGGKVL | | | KVL | | | | | WB |
| | | | KKI | | | | | | | | |
| 214 | DRB1_ | 632 | IDHILS | 3 | 1142 | ILSFGG | 1 | Sequence | 0.929631 | 0.16 | <= |
| | 1501 | | FGGKVL | | | KVL | | | | | SB |
| | | | KKI | | | | | | | | |
| 214 | DRB1_ | 632 | IDHILS | 3 | 1142 | ILSFGG | 1 | Sequence | 0.817948 | 0.38 | <= |
| | 1601 | | FGGKVL | | | KVL | | | | | SB |
| | | | KKI | | | | | | | | |
| 214 | DRB1_ | 632 | IDHILS | 3 | 1142 | ILSFGG | 1 | Sequence | 0.826473 | 0.12 | <= |
| | 1503 | | FGGKVL | | | KVL | | | | | SB |
| | | | KKI | | | | | | | | |
| 215 | DRB1_ | 633 | DHILSF | 2 | 1142 | ILSFGG | 1 | Sequence | 0.389469 | 4.33 | <= |
| | 0102 | | GGKVLK | | | KVL | | | | | WB |
| | | | KID | | | | | | | | |
| 215 | DRB1_ | 633 | DHILSF | 2 | 1142 | ILSFGG | 0.947 | Sequence | 0.433608 | 2.15 | <= |
| | 0103 | | GGKVLK | | | KVL | | | | | WB |
| | | | KID | | | | | | | | |
| 215 | DRB1_ | 633 | DHILSF | 2 | 1142 | ILSFGG | 1 | Sequence | 0.840672 | 0.41 | <= |
| | 1501 | | GGKVLK | | | KVL | | | | | SB |
| | | | KID | | | | | | | | |
| 215 | DRB1_ | 633 | DHILSF | 2 | 1142 | ILSFGG | 0.993 | Sequence | 0.662804 | 1.16 | <= |
| | 1601 | | GGKVLK | | | KVL | | | | | WB |
| | | | KID | | | | | | | | |
| 215 | DRB1_ | 633 | DHILSF | 2 | 1142 | ILSFGG | 1 | Sequence | 0.715699 | 0.3 | <= |
| | 1503 | | GGKVLK | | | KVL | | | | | SB |
| | | | KID | | | | | | | | |
| 216 | DRB1_ | 634 | HILSFG | 1 | 1142 | ILSFGG | 1 | Sequence | 0.321566 | 3.06 | <= |
| | 1501 | | GKVLKK | | | KVL | | | | | WB |
| | | | IDN | | | | | | | | |
| 216 | DRB1_ | 634 | HILSFG | 1 | 1142 | ILSFGG | 0.987 | Sequence | 0.280195 | 2.67 | <= |
| | 1503 | | GKVLKK | | | KVL | | | | | WB |
| | | | IDN | | | | | | | | |
| 220 | DRB1_ | 635 | FGGKVL | 4 | 1143 | VLKKID | 0.513 | Sequence | 0.378965 | 2.74 | <= |
| | 0402 | | KKIDND | | | NDV | | | | | WB |
| | | | VTI | | | | | | | | |
| 220 | DRB1_ | 635 | FGGKVL | 5 | 1144 | LKKIDN | 0.98 | Sequence | 0.486401 | 3.13 | <= |
| | 0403 | | KKIDND | | | DVT | | | | | WB |
| | | | VTI | | | | | | | | |
| 220 | DRB1_ | 635 | FGGKVL | 5 | 1144 | LKKIDN | 0.98 | Sequence | 0.433084 | 3.43 | <= |
| | 0404 | | KKIDND | | | DVT | | | | | WB |
| | | | VTI | | | | | | | | |
| 221 | DRB1_ | 636 | GGKVLK | 4 | 1144 | LKKIDN | 0.593 | Sequence | 0.394632 | 2.54 | <= |
| | 0402 | | KIDNDV | | | DVT | | | | | WB |
| | | | TIL | | | | | | | | |
| 221 | DRB1_ | 636 | GGKVLK | 4 | 1144 | LKKIDN | 0.987 | Sequence | 0.511095 | 2.81 | <= |
| | 0403 | | KIDNDV | | | DVT | | | | | WB |
| | | | TIL | | | | | | | | |
| 221 | DRB1_ | 636 | GGKVLK | 4 | 1144 | LKKIDN | 0.98 | Sequence | 0.483219 | 2.87 | <= |
| | 0404 | | KIDNDV | | | DVT | | | | | WB |
| | | | TIL | | | | | | | | |
| 222 | DRB1_ | 637 | GKVLKK | 3 | 1144 | LKKIDN | 0.947 | Sequence | 0.323501 | 4.12 | <= |
| | 0401 | | IDNDVT | | | DVT | | | | | WB |
| | | | ILE | | | | | | | | |
| 222 | DRB1_ | 637 | GKVLKK | 3 | 1144 | LKKIDN | 0.92 | Sequence | 0.409275 | 2.38 | <= |
| | 0402 | | IDNDVT | | | DVT | | | | | WB |
| | | | ILE | | | | | | | | |
| 222 | DRB1_ | 637 | GKVLKK | 3 | 1144 | LKKIDN | 0.947 | Sequence | 0.596731 | 1.86 | <= |
| | 0403 | | IDNDVT | | | DVT | | | | | WB |
| | | | ILE | | | | | | | | |
| 222 | DRB1_ | 637 | GKVLKK | 3 | 1144 | LKKIDN | 0.993 | Sequence | 0.594224 | 1.89 | <= |
| | 0404 | | IDNDVT | | | DVT | | | | | WB |
| | | | ILE | | | | | | | | |
| 222 | DRB1_ | 637 | GKVLKK | 3 | 1144 | LKKIDN | 0.98 | Sequence | 0.328543 | 4.83 | <= |
| | 0408 | | IDNDVT | | | DVT | | | | | WB |
| | | | ILE | | | | | | | | |
| 223 | DRB1_ | 638 | KVLKKI | 5 | 1145 | IDNDVT | 1 | Sequence | 0.529155 | 1.85 | <= |
| | 0301 | | DNDVTI | | | ILE | | | | | WB |
| | | | LEQ | | | | | | | | |
| 223 | DRB1_ | 638 | KVLKKI | 5 | 1145 | IDNDVT | 0.813 | Sequence | 0.184784 | 2.69 | <= |
| | 0305 | | DNDVTI | | | ILE | | | | | WB |
| | | | LEQ | | | | | | | | |
| 223 | DRB1_ | 638 | KVLKKI | 5 | 1145 | IDNDVT | 0.733 | Sequence | 0.285366 | 4.81 | <= |
| | 0401 | | DNDVTI | | | ILE | | | | | WB |
| | | | LEQ | | | | | | | | |
| 223 | DRB1_ | 638 | KVLKKI | 5 | 1145 | IDNDVT | 0.6 | Sequence | 0.52771 | 2.59 | <= |
| | 0403 | | DNDVTI | | | ILE | | | | | WB |
| | | | LEQ | | | | | | | | |
| 223 | DRB1_ | 638 | KVLKKI | 2 | 1144 | LKKIDN | 0.627 | Sequence | 0.340971 | 4.74 | <= |
| | 0404 | | DNDVTI | | | DVT | | | | | WB |
| | | | LEQ | | | | | | | | |
| 223 | DRB1_ | 638 | KVLKKI | 5 | 1145 | IDNDVT | 0.88 | Sequence | 0.297972 | 4.01 | <= |
| | 0405 | | DNDVTI | | | ILE | | | | | WB |
| | | | LEQ | | | | | | | | |
| 223 | DRB1_ | 638 | KVLKKI | 5 | 1145 | IDNDVT | 0.973 | Sequence | 0.337826 | 4.4 | <= |
| | 1303 | | DNDVTI | | | ILE | | | | | WB |
| | | | LEQ | | | | | | | | |
| 224 | DRB1_ | 639 | VLKKID | 4 | 1145 | IDNDVT | 1 | Sequence | 0.70227 | 0.98 | <= |
| | 0301 | | NDVTIL | | | ILE | | | | | SB |
| | | | EQA | | | | | | | | |
| 224 | DRB1_ | 639 | VLKKID | 4 | 1145 | IDNDVT | 0.973 | Sequence | 0.270742 | 1.7 | <= |
| | 0305 | | NDVTIL | | | ILE | | | | | WB |
| | | | EQA | | | | | | | | |
| 224 | DRB1_ | 639 | VLKKID | 4 | 1145 | IDNDVT | 1 | Sequence | 0.470534 | 2.35 | <= |
| | 0401 | | NDVTIL | | | ILE | | | | | WB |
| | | | EQA | | | | | | | | |
| 224 | DRB1_ | 639 | VLKKID | 4 | 1145 | IDNDVT | 0.987 | Sequence | 0.625549 | 1.62 | <= |
| | 0403 | | NDVTIL | | | ILE | | | | | WB |
| | | | EQA | | | | | | | | |
| 224 | DRB1_ | 639 | VLKKID | 4 | 1145 | IDNDVT | 0.967 | Sequence | 0.343374 | 4.7 | <= |
| | 0404 | | NDVTIL | | | ILE | | | | | WB |
| | | | EQA | | | | | | | | |
| 224 | DRB1_ | 639 | VLKKID | 4 | 1145 | IDNDVT | 1 | Sequence | 0.445908 | 2.27 | <= |
| | 0405 | | NDVTIL | | | ILE | | | | | WB |
| | | | EQA | | | | | | | | |
| 224 | DRB1_ | 639 | VLKKID | 4 | 1145 | IDNDVT | 1 | Sequence | 0.498069 | 2.42 | <= |
| | 1303 | | NDVTIL | | | ILE | | | | | WB |
| | | | EQA | | | | | | | | |
| 225 | DRB1_ | 640 | LKKIDN | 3 | 1145 | IDNDVT | 1 | Sequence | 0.697272 | 1 | <= |
| | 0301 | | DVTILE | | | ILE | | | | | SB |
| | | | QAF | | | | | | | | |
| 225 | DRB1_ | 640 | LKKIDN | 3 | 1145 | IDNDVT | 0.993 | Sequence | 0.26714 | 1.74 | <= |
| | 0305 | | DVTILE | | | ILE | | | | | WB |
| | | | QAF | | | | | | | | |
| 225 | DRB1_ | 640 | LKKIDN | 3 | 1145 | IDNDVT | 1 | Sequence | 0.470717 | 2.35 | <= |
| | 0401 | | DVTILE | | | ILE | | | | | WB |
| | | | QAF | | | | | | | | |
| 225 | DRB1_ | 640 | LKKIDN | 3 | 1145 | IDNDVT | 1 | Sequence | 0.617306 | 1.69 | <= |
| | 0403 | | DVTILE | | | ILE | | | | | WB |
| | | | QAF | | | | | | | | |
| 225 | DRB1_ | 640 | LKKIDN | 3 | 1145 | IDNDVT | 1 | Sequence | 0.348129 | 4.62 | <= |
| | 0404 | | DVTILE | | | ILE | | | | | WB |
| | | | QAF | | | | | | | | |
| 225 | DRB1_ | 640 | LKKIDN | 3 | 1145 | IDNDVT | 1 | Sequence | 0.426955 | 2.43 | <= |
| | 0405 | | DVTILE | | | ILE | | | | | WB |
| | | | QAF | | | | | | | | |
| 225 | DRB1_ | 640 | LKKIDN | 3 | 1145 | IDNDVT | 1 | Sequence | 0.465464 | 2.76 | <= |
| | 1303 | | DVTILE | | | ILE | | | | | WB |
| | | | QAF | | | | | | | | |
| 226 | DRB1_ | 641 | KKIDND | 2 | 1145 | IDNDVT | 1 | Sequence | 0.578719 | 1.58 | <= |
| | 0301 | | VTILEQ | | | ILE | | | | | WB |
| | | | AFA | | | | | | | | |
| 226 | DRB1_ | 641 | KKIDND | 2 | 1145 | IDNDVT | 0.987 | Sequence | 0.184653 | 2.69 | <= |
| | 0305 | | VTILEQ | | | ILE | | | | | WB |
| | | | AFA | | | | | | | | |
| 226 | DRB1_ | 641 | KKIDND | 2 | 1145 | IDNDVT | 0.987 | Sequence | 0.299944 | 4.53 | <= |
| | 0401 | | VTILEQ | | | ILE | | | | | WB |
| | | | AFA | | | | | | | | |
| 226 | DRB1_ | 641 | KKIDND | 2 | 1145 | IDNDVT | 0.98 | Sequence | 0.464619 | 3.42 | <= |
| | 0403 | | VTILEQ | | | ILE | | | | | WB |
| | | | AFA | | | | | | | | |
| 226 | DRB1_ | 641 | KKIDND | 2 | 1145 | IDNDVT | 0.987 | Sequence | 0.272306 | 4.46 | <= |
| | 0405 | | VTILEQ | | | ILE | | | | | WB |
| | | | AFA | | | | | | | | |
| 226 | DRB1_ | 641 | KKIDND | 2 | 1145 | IDNDVT | 1 | Sequence | 0.32866 | 4.55 | <= |
| | 1303 | | VTILEQ | | | ILE | | | | | WB |
| | | | AFA | | | | | | | | |
| 228 | DRB1_ | 642 | IDNDVT | 4 | 1146 | VTILEQ | 0.913 | Sequence | 0.322937 | 3.67 | <= |
| | 1201 | | ILEQAF | | | AFA | | | | | WB |
| | | | ATT | | | | | | | | |
| 229 | DRB1_ | 643 | DNDVTI | 3 | 1146 | VTILEQ | 0.967 | Sequence | 0.366255 | 2.87 | <= |
| | 1201 | | LEQAFA | | | AFA | | | | | WB |
| | | | TTS | | | | | | | | |
| 233 | DRB1_ | 644 | TILEQA | 6 | 1147 | FATTSK | 0.987 | Sequence | 0.538823 | 2.98 | <= |
| | 0801 | | FATTSK | | | IPE | | | | | WB |
| | | | IPE | | | | | | | | |
| 233 | DRB1_ | 644 | TILEQA | 6 | 1147 | FATTSK | 1 | Sequence | 0.777486 | 0.61 | <= |
| | 1303 | | FATTSK | | | IPE | | | | | SB |
| | | | IPE | | | | | | | | |
| 233 | DRB1_ | 644 | TILEQA | 6 | 1147 | FATTSK | 0.967 | Sequence | 0.457636 | 1.49 | <= |
| | 0803 | | FATTSK | | | IPE | | | | | WB |
| | | | IPE | | | | | | | | |
| 234 | DRB1_ | 645 | ILEQAF | 5 | 1147 | FATTSK | 0.973 | Sequence | 0.107755 | 4.51 | <= |
| | 0305 | | ATTSKI | | | IPE | | | | | WB |
| | | | PEP | | | | | | | | |
| 234 | DRB1_ | 645 | ILEQAF | 5 | 1147 | FATTSK | 1 | Sequence | 0.893068 | 0.2 | <= |
| | 0801 | | ATTSKI | | | IPE | | | | | SB |
| | | | PEP | | | | | | | | |
| 234 | DRB1_ | 645 | ILEQAF | 3 | 1148 | QAFATT | 0.607 | Sequence | 0.361377 | 4.74 | <= |
| | 0901 | | ATTSKI | | | SKI | | | | | WB |
| | | | PEP | | | | | | | | |
| 234 | DRB1_ | 645 | ILEQAF | 5 | 1147 | FATTSK | 1 | Sequence | 0.544513 | 2.66 | <= |
| | 1101 | | ATTSKI | | | IPE | | | | | WB |
| | | | PEP | | | | | | | | |
| 234 | DRB1_ | 645 | ILEQAF | 5 | 1147 | FATTSK | 0.987 | Sequence | 0.39126 | 3.09 | <= |
| | 1401 | | ATTSKI | | | IPE | | | | | WB |
| | | | PEP | | | | | | | | |
| 234 | DRB1_ | 645 | ILEQAF | 5 | 1147 | FATTSK | 0.987 | Sequence | 0.39126 | 3.09 | <= |
| | 1454 | | ATTSKI | | | IPE | | | | | WB |
| | | | PEP | | | | | | | | |
| 234 | DRB1_ | 645 | ILEQAF | 5 | 1147 | FATTSK | 0.94 | Sequence | 0.186525 | 3.58 | <= |
| | 1402 | | ATTSKI | | | IPE | | | | | WB |
| | | | PEP | | | | | | | | |
| 234 | DRB1_ | 645 | ILEQAF | 5 | 1147 | FATTSK | 1 | Sequence | 0.955725 | 0.04 | <= |
| | 1303 | | ATTSKI | | | IPE | | | | | SB |
| | | | PEP | | | | | | | | |
| 234 | DRB1_ | 645 | ILEQAF | 5 | 1147 | FATTSK | 0.993 | Sequence | 0.777236 | 0.09 | <= |
| | 0803 | | ATTSKI | | | IPE | | | | | SB |
| | | | PEP | | | | | | | | |
| 235 | DRB1_ | 646 | LEQAFA | 4 | 1147 | FATTSK | 0.987 | Sequence | 0.148339 | 3.35 | <= |
| | 0305 | | TTSKIP | | | IPE | | | | | WB |
| | | | EPY | | | | | | | | |
| 235 | DRB1_ | 646 | LEQAFA | 4 | 1147 | FATTSK | 1 | Sequence | 0.931361 | 0.09 | <= |
| | 0801 | | TTSKIP | | | IPE | | | | | SB |
| | | | EPY | | | | | | | | |
| 235 | DRB1_ | 646 | LEQAFA | 4 | 1147 | FATTSK | 1 | Sequence | 0.710451 | 1.47 | <= |
| | 1101 | | TTSKIP | | | IPE | | | | | WB |
| | | | EPY | | | | | | | | |
| 235 | DRB1_ | 646 | LEQAFA | 4 | 1147 | FATTSK | 1 | Sequence | 0.473015 | 1.99 | <= |
| | 1401 | | TTSKIP | | | IPE | | | | | WB |
| | | | EPY | | | | | | | | |
| 235 | DRB1_ | 646 | LEQAFA | 4 | 1147 | FATTSK | 1 | Sequence | 0.473015 | 1.99 | <= |
| | 1454 | | TTSKIP | | | IPE | | | | | WB |
| | | | EPY | | | | | | | | |
| 235 | DRB1_ | 646 | LEQAFA | 4 | 1147 | FATTSK | 0.987 | Sequence | 0.259928 | 1.64 | <= |
| | 1402 | | TTSKIP | | | IPE | | | | | WB |
| | | | EPY | | | | | | | | |
| 235 | DRB1_ | 646 | LEQAFA | 4 | 1147 | FATTSK | 1 | Sequence | 0.968079 | 0.01 | <= |
| | 1303 | | TTSKIP | | | IPE | | | | | SB |
| | | | EPY | | | | | | | | |
| 235 | DRB1_ | 646 | LEQAFA | 4 | 1147 | FATTSK | 0.973 | Sequence | 0.313152 | 4.43 | <= |
| | 1302 | | TTSKIP | | | IPE | | | | | WB |
| | | | EPY | | | | | | | | |
| 235 | DRB1_ | 646 | LEQAFA | 4 | 1147 | FATTSK | 1 | Sequence | 0.836134 | 0.04 | <= |
| | 0803 | | TTSKIP | | | IPE | | | | | SB |
| | | | EPY | | | | | | | | |
| 236 | DRB1_ | 647 | EQAFAT | 3 | 1147 | FATTSK | 0.993 | Sequence | 0.161225 | 3.07 | <= |
| | 0305 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.938154 | 0.08 | <= |
| | 0801 | | TSKIPE | | | IPE | | | | | SB |
| | | | PYY | | | | | | | | |
| 236 | DRB1_ | 647 | EQAFAT | 3 | 1147 | FATTSK | 1 | Sequence | 0.757859 | 1.18 | <= |
| | 1101 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.497684 | 1.76 | <= |
| | 1401 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.497684 | 1.76 | <= |
| | 1454 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.284191 | 1.3 | <= |
| | 1402 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.969903 | 0.01 | <= |
| | 1303 | | TSKIPE | | | IPE | | | | | SB |
| | | | PYY | | | | | | | | |
| 236 | DRB1_ | 647 | EQAFAT | 3 | 1147 | FATTSK | 0.993 | Sequence | 0.344778 | 3.49 | <= |
| | 1302 | | TSKIPE | | | IPE | | | | | WB |
| | | | PYY | | | | | | | | |
| 236 | DRB1_ | 647 | EQAFAT | 3 | 1147 | FATTSK | 1 | Sequence | 0.849673 | 0.03 | <= |
| | 0803 | | TSKIPE | | | IPE | | | | | SB |
| | | | PYY | | | | | | | | |
| 237 | DRB1_ | 648 | QAFATT | 2 | 1147 | FATTSK | 1 | Sequence | 0.827184 | 0.47 | <= |
| | 0801 | | SKIPEP | | | IPE | | | | | SB |
| | | | YYS | | | | | | | | |
| 237 | DRB1_ | 648 | QAFATT | 2 | 1147 | FATTSK | 1 | Sequence | 0.455564 | 3.57 | <= |
| | 1101 | | SKIPEP | | | IPE | | | | | WB |
| | | | YYS | | | | | | | | |
| 237 | DRB1_ | 648 | QAFATT | 2 | 1147 | FATTSK | 1 | Sequence | 0.927263 | 0.1 | <= |
| | 1303 | | SKIPEP | | | IPE | | | | | SB |
| | | | YYS | | | | | | | | |
| 237 | DRB1_ | 648 | QAFATT | 2 | 1147 | FATTSK | 1 | Sequence | 0.676363 | 0.25 | <= |
| | 0803 | | SKIPEP | | | IPE | | | | | SB |
| | | | YYS | | | | | | | | |
| 238 | DRB1_ | 649 | AFATTS | 1 | 1147 | FATTSK | 1 | Sequence | 0.621275 | 1.41 | <= |
| | 1303 | | KIPEPY | | | IPE | | | | | WB |
| | | | YSI | | | | | | | | |
| 242 | DRB1_ | 650 | TSKIPE | 4 | 1149 | PEPYYS | 0.993 | Sequence | 0.379776 | 2.51 | <= |
| | 1501 | | PYYSIK | | | IKA | | | | | WB |
| | | | ASK | | | | | | | | |
| 242 | DRB1_ | 650 | TSKIPE | 4 | 1149 | PEPYYS | 0.993 | Sequence | 0.282642 | 2.64 | <= |
| | 1503 | | PYYSIK | | | IKA | | | | | WB |
| | | | ASK | | | | | | | | |
| 243 | DRB1_ | 651 | SKIPEP | 6 | 1150 | YYSIKA | 1 | Sequence | 0.66796 | 1.52 | <= |
| | 0101 | | YYSIKA | | | SKI | | | | | WB |
| | | | SKI | | | | | | | | |
| 243 | DRB1_ | 651 | SKIPEP | 6 | 1150 | YYSIKA | 0.987 | Sequence | 0.349362 | 4.93 | <= |
| | 0102 | | YYSIKA | | | SKI | | | | | WB |
| | | | SKI | | | | | | | | |
| 243 | DRB1_ | 651 | SKIPEP | 6 | 1150 | YYSIKA | 1 | Sequence | 0.523255 | 1.44 | <= |
| | 0103 | | YYSIKA | | | SKI | | | | | WB |
| | | | SKI | | | | | | | | |
| 243 | DRB1_ | 651 | SKIPEP | 6 | 1150 | YYSIKA | 0.993 | Sequence | 0.854826 | 0.26 | <= |
| | 0701 | | YYSIKA | | | SKI | | | | | SB |
| | | | SKI | | | | | | | | |
| 243 | DRB1_ | 651 | SKIPEP | 6 | 1150 | YYSIKA | 1 | Sequence | 0.77206 | 0.31 | <= |
| | 0901 | | YYSIKA | | | SKI | | | | | SB |
| | | | SKI | | | | | | | | |
| 243 | DRB1_ | 651 | SKIPEP | 6 | 1150 | YYSIKA | 1 | Sequence | 0.549439 | 2.42 | <= |
| | 1001 | | YYSIKA | | | SKI | | | | | WB |
| | | | SKI | | | | | | | | |
| 243 | DRB1_ | 651 | SKIPEP | 3 | 1149 | PEPYYS | 0.967 | Sequence | 0.378597 | 2.52 | <= |
| | 1501 | | YYSIKA | | | IKA | | | | | WB |
| | | | SKI | | | | | | | | |
| 243 | DRB1_ | 651 | SKIPEP | 6 | 1150 | YYSIKA | 0.82 | Sequence | 0.519306 | 2.3 | <= |
| | 1601 | | YYSIKA | | | SKI | | | | | WB |
| | | | SKI | | | | | | | | |
| 243 | DRB1_ | 651 | SKIPEP | 3 | 1149 | PEPYYS | 0.873 | Sequence | 0.297364 | 2.45 | <= |
| | 1503 | | YYSIKA | | | IKA | | | | | WB |
| | | | SKI | | | | | | | | |
| 244 | DRB1_ | 652 | KIPEPY | 5 | 1150 | YYSIKA | 1 | Sequence | 0.951096 | 0.2 | <= |
| | 0101 | | YSIKAS | | | SKI | | | | | SB |
| | | | KIW | | | | | | | | |
| 244 | DRB1_ | 652 | KIPEPY | 5 | 1150 | YYSIKA | 1 | Sequence | 0.741858 | 1.22 | <= |
| | 0102 | | YSIKAS | | | SKI | | | | | WB |
| | | | KIW | | | | | | | | |
| 244 | DRB1_ | 652 | KIPEPY | 5 | 1150 | YYSIKA | 1 | Sequence | 0.880568 | 0.13 | <= |
| | 0103 | | YSIKAS | | | SKI | | | | | SB |
| | | | KIW | | | | | | | | |
| 244 | DRB1_ | 652 | KIPEPY | 5 | 1150 | YYSIKA | 1 | Sequence | 0.940445 | 0.07 | <= |
| | 0701 | | YSIKAS | | | SKI | | | | | SB |
| | | | KIW | | | | | | | | |
| 244 | DRB1_ | 652 | KIPEPY | 5 | 1150 | YYSIKA | 0.953 | Sequence | 0.457858 | 4.3 | <= |
| | 0801 | | YSIKAS | | | SKI | | | | | WB |
| | | | KIW | | | | | | | | |
| 244 | DRB1_ | 652 | KIPEPY | 5 | 1150 | YYSIKA | 1 | Sequence | 0.86029 | 0.09 | <= |
| | 0901 | | YSIKAS | | | SKI | | | | | SB |
| | | | KIW | | | | | | | | |
| 244 | DRB1_ | 652 | KIPEPY | 5 | 1150 | YYSIKA | 1 | Sequence | 0.819246 | 0.41 | <= |
| | 1001 | | YSIKAS | | | SKI | | | | | SB |
| | | | KIW | | | | | | | | |
| 244 | DRB1_ | 652 | KIPEPY | 2 | 1149 | PEPYYS | 0.613 | Sequence | 0.219619 | 4.58 | <= |
| | 1501 | | YSIKAS | | | IKA | | | | | WB |
| | | | KIW | | | | | | | | |
| 244 | DRB1_ | 652 | KIPEPY | 5 | 1150 | YYSIKA | 1 | Sequence | 0.859922 | 0.25 | <= |
| | 1601 | | YSIKAS | | | SKI | | | | | SB |
| | | | KIW | | | | | | | | |
| 244 | DRB1_ | 652 | KIPEPY | 5 | 1150 | YYSIKA | 0.54 | Sequence | 0.215408 | 3.76 | <= |
| | 1503 | | YSIKAS | | | SKI | | | | | WB |
| | | | KIW | | | | | | | | |
| 244 | DRB1_ | 652 | KIPEPY | 5 | 1150 | YYSIKA | 0.953 | Sequence | 0.170953 | 4.21 | <= |
| | 1402 | | YSIKAS | | | SKI | | | | | WB |
| | | | KIW | | | | | | | | |
| 244 | DRB1_ | 652 | KIPEPY | 5 | 1150 | YYSIKA | 0.933 | Sequence | 0.428337 | 1.84 | <= |
| | 0803 | | YSIKAS | | | SKI | | | | | WB |
| | | | KIW | | | | | | | | |
| 245 | DRB1_ | 653 | IPEPYY | 4 | 1150 | YYSIKA | 1 | Sequence | 0.984086 | 0.04 | <= |
| | 0101 | | SIKASK | | | SKI | | | | | SB |
| | | | IWA | | | | | | | | |
| 245 | DRB1_ | 653 | IPEPYY | 4 | 1150 | YYSIKA | 1 | Sequence | 0.860289 | 0.56 | <= |
| | 0102 | | SIKASK | | | SKI | | | | | SB |
| | | | IWA | | | | | | | | |
| 245 | DRB1_ | 653 | IPEPYY | 4 | 1150 | YYSIKA | 1 | Sequence | 0.956673 | 0.02 | <= |
| | 0103 | | SIKASK | | | SKI | | | | | SB |
| | | | IWA | | | | | | | | |
| 245 | DRB1_ | 653 | IPEPYY | 4 | 1150 | YYSIKA | 1 | Sequence | 0.326768 | 4.06 | <= |
| | 0401 | | SIKASK | | | SKI | | | | | WB |
| | | | IWA | | | | | | | | |
| 245 | DRB1_ | 653 | IPEPYY | 4 | 1150 | YYSIKA | 1 | Sequence | 0.280168 | 4.32 | <= |
| | 0405 | | SIKASK | | | SKI | | | | | WB |
| | | | IWA | | | | | | | | |
| 245 | DRB1_ | 653 | IPEPYY | 4 | 1150 | YYSIKA | 1 | Sequence | 0.962358 | 0.03 | <= |
| | 0701 | | SIKASK | | | SKI | | | | | SB |
| | | | IWA | | | | | | | | |
| 245 | DRB1_ | 653 | IPEPYY | 4 | 1150 | YYSIKA | 0.913 | Sequence | 0.592761 | 2.32 | <= |
| | 0801 | | SIKASK | | | SKI | | | | | WB |
| | | | IWA | | | | | | | | |
| 245 | DRB1_ | 653 | IPEPYY | 4 | 1150 | YYSIKA | 1 | Sequence | 0.896164 | 0.04 | <= |
| | 0901 | | SIKASK | | | SKI | | | | | SB |
| | | | IWA | | | | | | | | |
| 245 | DRB1_ | 653 | IPEPYY | 4 | 1150 | YYSIKA | 1 | Sequence | 0.891896 | 0.16 | <= |
| | 1001 | | SIKASK | | | SKI | | | | | SB |
| | | | IWA | | | | | | | | |
| 245 | DRB1_ | 653 | IPEPYY | 4 | 1150 | YYSIKA | 0.98 | Sequence | 0.467829 | 3.43 | <= |
| | 1101 | | SIKASK | | | SKI | | | | | WB |
| | | | IWA | | | | | | | | |
| 245 | DRB1_ | 653 | IPEPYY | 4 | 1150 | YYSIKA | 0.987 | Sequence | 0.326646 | 3.59 | <= |
| | 1201 | | SIKASK | | | SKI | | | | | WB |
| | | | IWA | | | | | | | | |
| 245 | DRB1_ | 653 | IPEPYY | 4 | 1150 | YYSIKA | 0.953 | Sequence | 0.323542 | 4.53 | <= |
| | 1401 | | SIKASK | | | SKI | | | | | WB |
| | | | IWA | | | | | | | | |
| 245 | DRB1_ | 653 | IPEPYY | 4 | 1150 | YYSIKA | 1 | Sequence | 0.94044 | 0.05 | <= |
| | 1601 | | SIKASK | | | SKI | | | | | SB |
| | | | IWA | | | | | | | | |
| 245 | DRB1_ | 653 | IPEPYY | 4 | 1150 | YYSIKA | 0.907 | Sequence | 0.224763 | 3.56 | <= |
| | 1503 | | SIKASK | | | SKI | | | | | WB |
| | | | IWA | | | | | | | | |
| 245 | DRB1_ | 653 | IPEPYY | 4 | 1150 | YYSIKA | 0.953 | Sequence | 0.323542 | 4.53 | <= |
| | 1454 | | SIKASK | | | SKI | | | | | WB |
| | | | IWA | | | | | | | | |
| 245 | DRB1_ | 653 | IPEPYY | 4 | 1150 | YYSIKA | 0.953 | Sequence | 0.252317 | 1.76 | <= |
| | 1402 | | SIKASK | | | SKI | | | | | WB |
| | | | IWA | | | | | | | | |
| 245 | DRB1_ | 653 | IPEPYY | 4 | 1150 | YYSIKA | 0.973 | Sequence | 0.324281 | 4.63 | <= |
| | 1303 | | SIKASK | | | SKI | | | | | WB |
| | | | IWA | | | | | | | | |
| 245 | DRB1_ | 653 | IPEPYY | 4 | 1150 | YYSIKA | 0.92 | Sequence | 0.372726 | 2.8 | <= |
| | 1302 | | SIKASK | | | SKI | | | | | WB |
| | | | IWA | | | | | | | | |
| 245 | DRB1_ | 653 | IPEPYY | 1 | 1150 | YYSIKA | 0.9 | Sequence | 0.545433 | 0.79 | <= |
| | 0803 | | SIKASK | | | SKI | | | | | SB |
| | | | IWA | | | | | | | | |
| 245 | DRB1_ | 653 | IPEPYY | 4 | 1150 | YYSIKA | 1 | Sequence | 0.379046 | 3.98 | <= |
| | 0408 | | SIKASK | | | SKI | | | | | WB |
| | | | IWA | | | | | | | | |
| 246 | DRB1_ | 654 | PEPYYS | 3 | 1150 | YYSIKA | 1 | Sequence | 0.977993 | 0.06 | <= |
| | 0101 | | IKASKI | | | SKI | | | | | SB |
| | | | WAL | | | | | | | | |
| 246 | DRB1_ | 654 | PEPYYS | 3 | 1150 | YYSIKA | 1 | Sequence | 0.833069 | 0.72 | <= |
| | 0102 | | IKASKI | | | SKI | | | | | SB |
| | | | WAL | | | | | | | | |
| 246 | DRB1_ | 654 | PEPYYS | 3 | 1150 | YYSIKA | 1 | Sequence | 0.94114 | 0.04 | <= |
| | 0103 | | IKASKI | | | SKI | | | | | SB |
| | | | WAL | | | | | | | | |
| 246 | DRB1_ | 654 | PEPYYS | 3 | 1150 | YYSIKA | 1 | Sequence | 0.947772 | 0.05 | <= |
| | 0701 | | IKASKI | | | SKI | | | | | SB |
| | | | WAL | | | | | | | | |
| 246 | DRB1_ | 654 | PEPYYS | 3 | 1150 | YYSIKA | 0.853 | Sequence | 0.510212 | 3.41 | <= |
| | 0801 | | IKASKI | | | SKI | | | | | WB |
| | | | WAL | | | | | | | | |
| 246 | DRB1_ | 654 | PEPYYS | 3 | 1150 | YYSIKA | 0.967 | Sequence | 0.875657 | 0.07 | <= |
| | 0901 | | IKASKI | | | SKI | | | | | SB |
| | | | WAL | | | | | | | | |
| 246 | DRB1_ | 654 | PEPYYS | 3 | 1150 | YYSIKA | 1 | Sequence | 0.865531 | 0.24 | <= |
| | 1001 | | IKASKI | | | SKI | | | | | SB |
| | | | WAL | | | | | | | | |
| 246 | DRB1_ | 654 | PEPYYS | 3 | 1150 | YYSIKA | 0.973 | Sequence | 0.382555 | 4.44 | <= |
| | 1101 | | IKASKI | | | SKI | | | | | WB |
| | | | WAL | | | | | | | | |
| 246 | DRB1_ | 654 | PEPYYS | 3 | 1150 | YYSIKA | 0.947 | Sequence | 0.298719 | 4.24 | <= |
| | 1201 | | IKASKI | | | SKI | | | | | WB |
| | | | WAL | | | | | | | | |
| 246 | DRB1_ | 654 | PEPYYS | 3 | 1150 | YYSIKA | 1 | Sequence | 0.919515 | 0.09 | <= |
| | 1601 | | IKASKI | | | SKI | | | | | SB |
| | | | WAL | | | | | | | | |
| 246 | DRB1_ | 654 | PEPYYS | 3 | 1150 | YYSIKA | 0.94 | Sequence | 0.193846 | 4.27 | <= |
| | 1503 | | IKASKI | | | SKI | | | | | WB |
| | | | WAL | | | | | | | | |
| 246 | DRB1_ | 654 | PEPYYS | 3 | 1150 | YYSIKA | 0.947 | Sequence | 0.223424 | 2.41 | <= |
| | 1402 | | IKASKI | | | SKI | | | | | WB |
| | | | WAL | | | | | | | | |
| 246 | DRB1_ | 654 | PEPYYS | 3 | 1150 | YYSIKA | 0.887 | Sequence | 0.329396 | 3.94 | <= |
| | 1302 | | IKASKI | | | SKI | | | | | WB |
| | | | WAL | | | | | | | | |
| 246 | DRB1_ | 654 | PEPYYS | 3 | 1150 | YYSIKA | 0.867 | Sequence | 0.487456 | 1.21 | <= |
| | 0803 | | IKASKI | | | SKI | | | | | WB |
| | | | WAL | | | | | | | | |
| 247 | DRB1_ | 655 | EPYYSI | 2 | 1150 | YYSIKA | 1 | Sequence | 0.864486 | 0.57 | <= |
| | 0101 | | KASKIW | | | SKI | | | | | SB |
| | | | ALP | | | | | | | | |
| 247 | DRB1_ | 655 | EPYYSI | 2 | 1150 | YYSIKA | 0.96 | Sequence | 0.577141 | 2.35 | <= |
| | 0102 | | KASKIW | | | SKI | | | | | WB |
| | | | ALP | | | | | | | | |
| 247 | DRB1_ | 655 | EPYYSI | 2 | 1150 | YYSIKA | 0.993 | Sequence | 0.769317 | 0.4 | <= |
| | 0103 | | KASKIW | | | SKI | | | | | SB |
| | | | ALP | | | | | | | | |
| 247 | DRB1_ | 655 | EPYYSI | 2 | 1150 | YYSIKA | 0.933 | Sequence | 0.820657 | 0.35 | <= |
| | 0701 | | KASKIW | | | SKI | | | | | SB |
| | | | ALP | | | | | | | | |
| 247 | DRB1_ | 655 | EPYYSI | 2 | 1150 | YYSIKA | 0.807 | Sequence | 0.789381 | 0.26 | <= |
| | 0901 | | KASKIW | | | SKI | | | | | SB |
| | | | ALP | | | | | | | | |
| 247 | DRB1_ | 655 | EPYYSI | 2 | 1150 | YYSIKA | 0.973 | Sequence | 0.6428 | 1.53 | <= |
| | 1001 | | KASKIW | | | SKI | | | | | WB |
| | | | ALP | | | | | | | | |
| 247 | DRB1_ | 655 | EPYYSI | 2 | 1150 | YYSIKA | 0.993 | Sequence | 0.657744 | 1.18 | <= |
| | 1601 | | KASKIW | | | SKI | | | | | WB |
| | | | ALP | | | | | | | | |
| 247 | DRB1_ | 655 | EPYYSI | 2 | 1150 | YYSIKA | 0.547 | Sequence | 0.307339 | 4.25 | <= |
| | 0803 | | KASKIW | | | SKI | | | | | WB |
| | | | ALP | | | | | | | | |
| 248 | DRB1_ | 656 | PYYSIK | 1 | 1150 | YYSIKA | 0.94 | Sequence | 0.354919 | 3.86 | <= |
| | 0101 | | ASKIWA | | | SKI | | | | | WB |
| | | | LPS | | | | | | | | |
| 248 | DRB1_ | 656 | PYYSIK | 1 | 1150 | YYSIKA | 0.8 | Sequence | 0.302515 | 3.75 | <= |
| | 0103 | | ASKIWA | | | SKI | | | | | WB |
| | | | LPS | | | | | | | | |
| 248 | DRB1_ | 656 | PYYSIK | 4 | 1151 | IKASKI | 0.673 | Sequence | 0.523892 | 1.62 | <= |
| | 0701 | | ASKIWA | | | WAL | | | | | WB |
| | | | LPS | | | | | | | | |
| 248 | DRB1_ | 656 | PYYSIK | 4 | 1151 | IKASKI | 0.507 | Sequence | 0.613452 | 1.13 | <= |
| | 0901 | | ASKIWA | | | WAL | | | | | WB |
| | | | LPS | | | | | | | | |
| 249 | DRB1_ | 657 | YYSIKA | 3 | 1151 | IKASKI | 1 | Sequence | 0.50192 | 1.77 | <= |
| | 0701 | | SKIWAL | | | WAL | | | | | WB |
| | | | PSG | | | | | | | | |
| 249 | DRB1_ | 657 | YYSIKA | 3 | 1151 | IKASKI | 0.973 | Sequence | 0.512273 | 2.1 | <= |
| | 0901 | | SKIWAL | | | WAL | | | | | WB |
| | | | PSG | | | | | | | | |
| 249 | DRB1_ | 657 | YYSIKA | 3 | 1151 | IKASKI | 0.987 | Sequence | 0.328589 | 4.41 | <= |
| | 1401 | | SKIWAL | | | WAL | | | | | WB |
| | | | PSG | | | | | | | | |
| 249 | DRB1_ | 657 | YYSIKA | 3 | 1151 | IKASKI | 0.987 | Sequence | 0.328589 | 4.41 | <= |
| | 1454 | | SKIWAL | | | WAL | | | | | WB |
| | | | PSG | | | | | | | | |
| 250 | DRB1_ | 658 | YSIKAS | 2 | 1151 | IKASKI | 1 | Sequence | 0.306043 | 3.79 | <= |
| | 0701 | | KIWALP | | | WAL | | | | | WB |
| | | | SGD | | | | | | | | |
| 250 | DRB1_ | 658 | YSIKAS | 2 | 1151 | IKASKI | 0.953 | Sequence | 0.382496 | 4.24 | <= |
| | 0901 | | KIWALP | | | WAL | | | | | WB |
| | | | SGD | | | | | | | | |
| 251 | DRB1_ | 659 | SIKASK | 6 | 1152 | IWALPS | 0.86 | Sequence | 0.290157 | 4.45 | <= |
| | 1201 | | IWALPS | | | GDF | | | | | WB |
| | | | GDF | | | | | | | | |
| 252 | DRB1_ | 660 | IKASKI | 5 | 1152 | IWALPS | 1 | Sequence | 0.643454 | 0.59 | <= |
| | 1201 | | WALPSG | | | GDF | | | | | SB |
| | | | DFG | | | | | | | | |
| 253 | DRB1_ | 661 | KASKIW | 4 | 1152 | IWALPS | 1 | Sequence | 0.711944 | 0.36 | <= |
| | 1201 | | ALPSGD | | | GDF | | | | | SB |
| | | | FGF | | | | | | | | |
| 254 | DRB1_ | 662 | ASKIWA | 3 | 1152 | IWALPS | 1 | Sequence | 0.68282 | 0.45 | <= |
| | 1201 | | LPSGDF | | | GDF | | | | | SB |
| | | | GFL | | | | | | | | |
| 255 | DRB1_ | 663 | SKIWAL | 2 | 1152 | IWALPS | 0.987 | Sequence | 0.481675 | 1.5 | <= |
| | 1201 | | PSGDFG | | | GDF | | | | | WB |
| | | | FLN | | | | | | | | |
| 260 | DRB1_ | 664 | LPSGDF | 5 | 1153 | FGFLNA | 0.993 | Sequence | 0.319154 | 3.76 | <= |
| | 1201 | | GFLNAI | | | IFY | | | | | WB |
| | | | FYM | | | | | | | | |
| 261 | DRB1_ | 665 | PSGDFG | 4 | 1153 | FGFLNA | 0.993 | Sequence | 0.443549 | 1.85 | <= |
| | 1201 | | FLNAIF | | | IFY | | | | | WB |
| | | | YMG | | | | | | | | |
| 262 | DRB1_ | 666 | SGDFGF | 3 | 1153 | FGFLNA | 1 | Sequence | 0.401392 | 3.39 | <= |
| | 0101 | | LNAIFY | | | IFY | | | | | WB |
| | | | MGR | | | | | | | | |
| 262 | DRB1_ | 666 | SGDFGF | 3 | 1153 | FGFLNA | 1 | Sequence | 0.553555 | 1.02 | <= |
| | 1201 | | LNAIFY | | | IFY | | | | | WB |
| | | | MGR | | | | | | | | |
| 263 | DRB1_ | 667 | GDFGFL | 2 | 1153 | FGFLNA | 0.987 | Sequence | 0.375776 | 2.73 | <= |
| | 1201 | | NAIFYM | | | IFY | | | | | WB |
| | | | GRV | | | | | | | | |
| 265 | DRB1_ | 668 | FGFLNA | 6 | 1154 | IFYMGR | 1 | Sequence | 0.55561 | 1.26 | <= |
| | 1401 | | IFYMGR | | | VPV | | | | | WB |
| | | | VPV | | | | | | | | |
| 265 | DRB1_ | 668 | FGFLNA | 6 | 1154 | IFYMGR | 1 | Sequence | 0.55561 | 1.26 | <= |
| | 1454 | | IFYMGR | | | VPV | | | | | WB |
| | | | VPV | | | | | | | | |
| 266 | DRB1_ | 669 | GFLNAI | 5 | 1154 | IFYMGR | 0.993 | Sequence | 0.379674 | 4.47 | <= |
| | 1101 | | FYMGRV | | | VPV | | | | | WB |
| | | | PVF | | | | | | | | |
| 266 | DRB1_ | 669 | GFLNAI | 5 | 1154 | IFYMGR | 1 | Sequence | 0.568016 | 2.69 | <= |
| | 1104 | | FYMGRV | | | VPV | | | | | WB |
| | | | PVF | | | | | | | | |
| 266 | DRB1_ | 669 | GFLNAI | 5 | 1154 | IFYMGR | 0.913 | Sequence | 0.277333 | 4.8 | <= |
| | 1201 | | FYMGRV | | | VPV | | | | | WB |
| | | | PVF | | | | | | | | |
| 266 | DRB1_ | 669 | GFLNAI | 5 | 1154 | IFYMGR | 1 | Sequence | 0.695451 | 0.51 | <= |
| | 1401 | | FYMGRV | | | VPV | | | | | SB |
| | | | PVF | | | | | | | | |
| 266 | DRB1_ | 669 | GFLNAI | 5 | 1154 | IFYMGR | 1 | Sequence | 0.695451 | 0.51 | <= |
| | 1454 | | FYMGRV | | | VPV | | | | | SB |
| | | | PVF | | | | | | | | |
| 266 | DRB1_ | 669 | GFLNAI | 5 | 1154 | IFYMGR | 0.967 | Sequence | 0.186782 | 3.57 | <= |
| | 1402 | | FYMGRV | | | VPV | | | | | WB |
| | | | PVF | | | | | | | | |
| 267 | DRB1_ | 670 | FLNAIF | 4 | 1154 | IFYMGR | 1 | Sequence | 0.48684 | 3.21 | <= |
| | 1101 | | YMGRVP | | | VPV | | | | | WB |
| | | | VFY | | | | | | | | |
| 267 | DRB1_ | 670 | FLNAIF | 4 | 1154 | IFYMGR | 1 | Sequence | 0.644297 | 1.98 | <= |
| | 1104 | | YMGRVP | | | VPV | | | | | WB |
| | | | VFY | | | | | | | | |
| 267 | DRB1_ | 670 | FLNAIF | 4 | 1154 | IFYMGR | 0.913 | Sequence | 0.321056 | 3.72 | <= |
| | 1201 | | YMGRVP | | | VPV | | | | | WB |
| | | | VFY | | | | | | | | |
| 267 | DRB1_ | 670 | FLNAIF | 4 | 1154 | IFYMGR | 0.993 | Sequence | 0.462679 | 4.32 | <= |
| | 1301 | | YMGRVP | | | VPV | | | | | WB |
| | | | VFY | | | | | | | | |
| 267 | DRB1_ | 670 | FLNAIF | 4 | 1154 | IFYMGR | 0.993 | Sequence | 0.718131 | 0.43 | <= |
| | 1401 | | YMGRVP | | | VPV | | | | | SB |
| | | | VFY | | | | | | | | |
| 267 | DRB1_ | 670 | FLNAIF | 4 | 1154 | IFYMGR | 0.993 | Sequence | 0.718131 | 0.43 | <= |
| | 1454 | | YMGRVP | | | VPV | | | | | SB |
| | | | VFY | | | | | | | | |
| 267 | DRB1_ | 670 | FLNAIF | 4 | 1154 | IFYMGR | 0.92 | Sequence | 0.261447 | 1.61 | <= |
| | 1402 | | YMGRVP | | | VPV | | | | | WB |
| | | | VFY | | | | | | | | |
| 268 | DRB1_ | 671 | LNAIFY | 3 | 1154 | IFYMGR | 0.993 | Sequence | 0.397159 | 4.26 | <= |
| | 1101 | | MGRVPV | | | VPV | | | | | WB |
| | | | FYI | | | | | | | | |
| 268 | DRB1_ | 671 | LNAIFY | 3 | 1154 | IFYMGR | 1 | Sequence | 0.566694 | 2.7 | <= |
| | 1104 | | MGRVPV | | | VPV | | | | | WB |
| | | | FYI | | | | | | | | |
| 268 | DRB1_ | 671 | LNAIFY | 3 | 1154 | IFYMGR | 0.84 | Sequence | 0.288906 | 4.48 | <= |
| | 1201 | | MGRVPV | | | VPV | | | | | WB |
| | | | FYI | | | | | | | | |
| 268 | DRB1_ | 671 | LNAIFY | 3 | 1154 | IFYMGR | 0.987 | Sequence | 0.670871 | 0.59 | <= |
| | 1401 | | MGRVPV | | | VPV | | | | | SB |
| | | | FYI | | | | | | | | |
| 268 | DRB1_ | 671 | LNAIFY | 3 | 1154 | IFYMGR | 0.987 | Sequence | 0.670871 | 0.59 | <= |
| | 1454 | | MGRVPV | | | VPV | | | | | SB |
| | | | FYI | | | | | | | | |
| 268 | DRB1_ | 671 | LNAIFY | 3 | 1154 | IFYMGR | 0.88 | Sequence | 0.247757 | 1.84 | <= |
| | 1402 | | MGRVPV | | | VPV | | | | | WB |
| | | | FYI | | | | | | | | |
| 269 | DRB1_ | 672 | NAIFYM | 2 | 1154 | IFYMGR | 0.993 | Sequence | 0.49526 | 3.49 | <= |
| | 1104 | | GRVPVF | | | VPV | | | | | WB |
| | | | YIP | | | | | | | | |
| 269 | DRB1_ | 672 | NAIFYM | 2 | 1154 | IFYMGR | 0.987 | Sequence | 0.605223 | 0.93 | <= |
| | 1401 | | GRVPVF | | | VPV | | | | | SB |
| | | | YIP | | | | | | | | |
| 269 | DRB1_ | 672 | NAIFYM | 2 | 1154 | IFYMGR | 0.987 | Sequence | 0.605223 | 0.93 | <= |
| | 1454 | | GRVPVF | | | VPV | | | | | SB |
| | | | YIP | | | | | | | | |
| 269 | DRB1_ | 672 | NAIFYM | 2 | 1154 | IFYMGR | 0.767 | Sequence | 0.186205 | 3.59 | <= |
| | 1402 | | GRVPVF | | | VPV | | | | | WB |
| | | | YIP | | | | | | | | |
| 281 | DRB1_ | 673 | YIPFFF | 3 | 1155 | FFFRPG | 0.947 | Sequence | 0.240113 | 4.95 | <= |
| | 0103 | | RPGDSL | | | DSL | | | | | WB |
| | | | FFN | | | | | | | | |
| 282 | DRB1_ | 674 | IPFFFR | 4 | 1156 | FRPGDS | 0.96 | Sequence | 0.375503 | 4.39 | <= |
| | 0901 | | PGDSLF | | | LFF | | | | | WB |
| | | | FNP | | | | | | | | |
| 283 | DRB1_ | 675 | PFFFRP | 3 | 1156 | FRPGDS | 0.987 | Sequence | 0.414932 | 3.58 | <= |
| | 0901 | | GDSLFF | | | LFF | | | | | WB |
| | | | NPS | | | | | | | | |
| 287 | DRB1_ | 676 | RPGDSL | 5 | 1157 | LFFNPS | 0.987 | Sequence | 0.308347 | 4.91 | <= |
| | 1401 | | FFNPSL | | | LGL | | | | | WB |
| | | | GLN | | | | | | | | |
| 287 | DRB1_ | 676 | RPGDSL | 5 | 1157 | LFFNPS | 0.987 | Sequence | 0.308347 | 4.91 | <= |
| | 1454 | | FFNPSL | | | LGL | | | | | WB |
| | | | GLN | | | | | | | | |
| 287 | DRB1_ | 676 | RPGDSL | 5 | 1157 | LFFNPS | 1 | Sequence | 0.480852 | 1.23 | <= |
| | 1302 | | FFNPSL | | | LGL | | | | | WB |
| | | | GLN | | | | | | | | |
| 288 | DRB1_ | 677 | PGDSLF | 4 | 1157 | LFFNPS | 0.98 | Sequence | 0.102623 | 4.73 | <= |
| | 0305 | | FNPSLG | | | LGL | | | | | WB |
| | | | LNP | | | | | | | | |
| 288 | DRB1_ | 677 | PGDSLF | 4 | 1157 | LFFNPS | 0.993 | Sequence | 0.450967 | 2.25 | <= |
| | 1401 | | FNPSLG | | | LGL | | | | | WB |
| | | | LNP | | | | | | | | |
| 288 | DRB1_ | 677 | PGDSLF | 4 | 1157 | LFFNPS | 0.993 | Sequence | 0.450967 | 2.25 | <= |
| | 1454 | | FNPSLG | | | LGL | | | | | WB |
| | | | LNP | | | | | | | | |
| 288 | DRB1_ | 677 | PGDSLF | 4 | 1157 | LFFNPS | 0.993 | Sequence | 0.238367 | 2.03 | <= |
| | 1402 | | FNPSLG | | | LGL | | | | | WB |
| | | | LNP | | | | | | | | |
| 288 | DRB1_ | 677 | PGDSLF | 4 | 1157 | LFFNPS | 1 | Sequence | 0.646815 | 0.22 | <= |
| | 1302 | | FNPSLG | | | LGL | | | | | SB |
| | | | LNP | | | | | | | | |
| 289 | DRB1_ | 678 | GDSLFF | 3 | 1157 | LFFNPS | 0.96 | Sequence | 0.257615 | 4.55 | <= |
| | 0103 | | NPSLGL | | | LGL | | | | | WB |
| | | | NPR | | | | | | | | |
| 289 | DRB1_ | 678 | GDSLFF | 3 | 1157 | LFFNPS | 0.987 | Sequence | 0.147338 | 3.37 | <= |
| | 0305 | | NPSLGL | | | LGL | | | | | WB |
| | | | NPR | | | | | | | | |
| 289 | DRB1_ | 678 | GDSLFF | 3 | 1157 | LFFNPS | 0.927 | Sequence | 0.271183 | 4.36 | <= |
| | 0701 | | NPSLGL | | | LGL | | | | | WB |
| | | | NPR | | | | | | | | |
| 289 | DRB1_ | 678 | GDSLFF | 3 | 1157 | LFFNPS | 0.993 | Sequence | 0.535342 | 1.42 | <= |
| | 1401 | | NPSLGL | | | LGL | | | | | WB |
| | | | NPR | | | | | | | | |
| 289 | DRB1_ | 678 | GDSLFF | 3 | 1157 | LFFNPS | 0.987 | Sequence | 0.26523 | 3.76 | <= |
| | 1501 | | NPSLGL | | | LGL | | | | | WB |
| | | | NPR | | | | | | | | |
| 289 | DRB1_ | 678 | GDSLFF | 3 | 1157 | LFFNPS | 0.993 | Sequence | 0.355885 | 4.37 | <= |
| | 1601 | | NPSLGL | | | LGL | | | | | WB |
| | | | NPR | | | | | | | | |
| 289 | DRB1_ | 678 | GDSLFF | 3 | 1157 | LFFNPS | 0.987 | Sequence | 0.217654 | 3.71 | <= |
| | 1503 | | NPSLGL | | | LGL | | | | | WB |
| | | | NPR | | | | | | | | |
| 289 | DRB1_ | 678 | GDSLFF | 3 | 1157 | LFFNPS | 0.993 | Sequence | 0.535342 | 1.42 | <= |
| | 1454 | | NPSLGL | | | LGL | | | | | WB |
| | | | NPR | | | | | | | | |
| 289 | DRB1_ | 678 | GDSLFF | 3 | 1157 | LFFNPS | 0.993 | Sequence | 0.323086 | 0.88 | <= |
| | 1402 | | NPSLGL | | | LGL | | | | | SB |
| | | | NPR | | | | | | | | |
| 289 | DRB1_ | 678 | GDSLFF | 3 | 1157 | LFFNPS | 1 | Sequence | 0.727278 | 0.08 | <= |
| | 1302 | | NPSLGL | | | LGL | | | | | SB |
| | | | NPR | | | | | | | | |
| 290 | DRB1_ | 679 | DSLFFN | 2 | 1157 | LFFNPS | 0.96 | Sequence | 0.306996 | 4.95 | <= |
| | 1401 | | PSLGLN | | | LGL | | | | | WB |
| | | | PRK | | | | | | | | |
| 290 | DRB1_ | 679 | DSLFFN | 2 | 1157 | LFFNPS | 0.96 | Sequence | 0.306996 | 4.95 | <= |
| | 1454 | | PSLGLN | | | LGL | | | | | WB |
| | | | PRK | | | | | | | | |
| 290 | DRB1_ | 679 | DSLFFN | 2 | 1157 | LFFNPS | 0.993 | Sequence | 0.54712 | 0.66 | <= |
| | 1302 | | PSLGLN | | | LGL | | | | | SB |
| | | | PRK | | | | | | | | |
| 296 | DRB1_ | 680 | PSLGLN | 4 | 1158 | LNPRKG | 0.993 | Sequence | 0.375077 | 2.73 | <= |
| | 0103 | | PRKGFA | | | FAV | | | | | WB |
| | | | VFN | | | | | | | | |
| 297 | DRB1_ | 681 | SLGLNP | 3 | 1158 | LNPRKG | 1 | Sequence | 0.438084 | 2.11 | <= |
| | 0103 | | RKGFAV | | | FAV | | | | | WB |
| | | | FNT | | | | | | | | |
| 301 | DRB1_ | 682 | NPRKGF | 5 | 1159 | FAVFNT | 1 | Sequence | 0.4264 | 2.15 | <= |
| | 1501 | | AVFNTV | | | VYL | | | | | WB |
| | | | YLF | | | | | | | | |
| 301 | DRB1_ | 682 | NPRKGF | 5 | 1159 | FAVFNT | 1 | Sequence | 0.628256 | 1.39 | <= |
| | 1601 | | AVFNTV | | | VYL | | | | | WB |
| | | | YLF | | | | | | | | |
| 301 | DRB1_ | 682 | NPRKGF | 5 | 1159 | FAVFNT | 0.98 | Sequence | 0.23814 | 3.32 | <= |
| | 1503 | | AVFNTV | | | VYL | | | | | WB |
| | | | YLF | | | | | | | | |
| 302 | DRB1_ | 683 | PRKGFA | 4 | 1159 | FAVFNT | 0.993 | Sequence | 0.321644 | 4.24 | <= |
| | 0101 | | VFNTVY | | | VYL | | | | | WB |
| | | | LFG | | | | | | | | |
| 302 | DRB1_ | 683 | PRKGFA | 4 | 1159 | FAVFNT | 0.98 | Sequence | 0.238012 | 4.99 | <= |
| | 0103 | | VFNTVY | | | VYL | | | | | WB |
| | | | LFG | | | | | | | | |
| 302 | DRB1_ | 683 | PRKGFA | 4 | 1159 | FAVFNT | 0.993 | Sequence | 0.361347 | 3.11 | <= |
| | 0405 | | VFNTVY | | | VYL | | | | | WB |
| | | | LFG | | | | | | | | |
| 302 | DRB1_ | 683 | PRKGFA | 4 | 1159 | FAVFNT | 0.987 | Sequence | 0.294104 | 3.97 | <= |
| | 0701 | | VFNTVY | | | VYL | | | | | WB |
| | | | LFG | | | | | | | | |
| 302 | DRB1_ | 683 | PRKGFA | 4 | 1159 | FAVFNT | 1 | Sequence | 0.61054 | 1.16 | <= |
| | 1501 | | VFNTVY | | | VYL | | | | | WB |
| | | | LFG | | | | | | | | |
| 302 | DRB1_ | 683 | PRKGFA | 4 | 1159 | FAVFNT | 1 | Sequence | 0.823181 | 0.36 | <= |
| | 1601 | | VFNTVY | | | VYL | | | | | SB |
| | | | LFG | | | | | | | | |
| 302 | DRBI 1503 | 683 | PRKGFA | 4 | 1159 | FAVFNT | 1 | Sequence | 0.380418 | 1.65 | <= |
| | | | VFNTVY | | | VYL | | | | | WB |
| | | | LFG | | | | | | | | |
| 302 | DRB1_ | 683 | PRKGFA | 4 | 1159 | FAVFNT | 0.927 | Sequence | 0.313238 | 4.08 | <= |
| | 0803 | | VFNTVY | | | VYL | | | | | WB |
| | | | LFG | | | | | | | | |
| 302 | DRB1_ | 683 | PRKGFA | 4 | 1159 | FAVFNT | 1 | Sequence | 0.455876 | 3.01 | <= |
| | 0408 | | VFNTVY | | | VYL | | | | | WB |
| | | | LFG | | | | | | | | |
| 303 | DRB1_ | 684 | RKGFAV | 3 | 1159 | FAVFNT | 1 | Sequence | 0.468411 | 2.8 | <= |
| | 0101 | | FNTVYL | | | VYL | | | | | WB |
| | | | FGN | | | | | | | | |
| 303 | DRB1_ | 684 | RKGFAV | 3 | 1159 | FAVFNT | 0.993 | Sequence | 0.338364 | 3.2 | <= |
| | 0103 | | FNTVYL | | | VYL | | | | | WB |
| | | | FGN | | | | | | | | |
| 303 | DRB1_ | 684 | RKGFAV | 3 | 1159 | FAVFNT | 1 | Sequence | 0.323494 | 4.12 | <= |
| | 0401 | | FNTVYL | | | VYL | | | | | WB |
| | | | FGN | | | | | | | | |
| 303 | DRB1_ | 684 | RKGFAV | 3 | 1159 | FAVFNT | 1 | Sequence | 0.40171 | 3.84 | <= |
| | 0404 | | FNTVYL | | | VYL | | | | | WB |
| | | | FGN | | | | | | | | |
| 303 | DRB1_ | 684 | RKGFAV | 3 | 1159 | FAVFNT | 1 | Sequence | 0.44935 | 2.24 | <= |
| | 0405 | | FNTVYL | | | VYL | | | | | WB |
| | | | FGN | | | | | | | | |
| 303 | DRB1_ | 684 | RKGFAV | 3 | 1159 | FAVFNT | 0.98 | Sequence | 0.380772 | 2.8 | <= |
| | 0701 | | FNTVYL | | | VYL | | | | | WB |
| | | | FGN | | | | | | | | |
| 303 | DRB1_ | 684 | RKGFAV | 3 | 1159 | FAVFNT | 0.96 | Sequence | 0.392363 | 4.03 | <= |
| | 0901 | | FNTVYL | | | VYL | | | | | WB |
| | | | FGN | | | | | | | | |
| 303 | DRB1_ | 684 | RKGFAV | 3 | 1159 | FAVFNT | 1 | Sequence | 0.705712 | 0.79 | <= |
| | 1501 | | FNTVYL | | | VYL | | | | | SB |
| | | | FGN | | | | | | | | |
| 303 | DRB1_ | 684 | RKGFAV | 3 | 1159 | FAVFNT | 1 | Sequence | 0.894368 | 0.15 | <= |
| | 1601 | | FNTVYL | | | VYL | | | | | SB |
| | | | FGN | | | | | | | | |
| 303 | DRB1_ | 684 | RKGFAV | 3 | 1159 | FAVFNT | 1 | Sequence | 0.481379 | 1.05 | <= |
| | 1503 | | FNTVYL | | | VYL | | | | | WB |
| | | | FGN | | | | | | | | |
| 303 | DRB1_ | 684 | RKGFAV | 3 | 1159 | FAVFNT | 0.933 | Sequence | 0.3901 | 2.41 | <= |
| | 0803 | | FNTVYL | | | VYL | | | | | WB |
| | | | FGN | | | | | | | | |
| 303 | DRB1_ | 684 | RKGFAV | 3 | 1159 | FAVFNT | 1 | Sequence | 0.553174 | 2.03 | <= |
| | 0408 | | FNTVYL | | | VYL | | | | | WB |
| | | | FGN | | | | | | | | |
| 304 | DRB1_ | 685 | KGFAVF | 2 | 1159 | FAVFNT | 0.993 | Sequence | 0.413985 | 2.25 | <= |
| | 1501 | | NTVYLF | | | VYL | | | | | WB |
| | | | GNK | | | | | | | | |
| 304 | DRB1_ | 685 | KGFAVF | 2 | 1159 | FAVFNT | 1 | Sequence | 0.602203 | 1.59 | <= |
| | 1601 | | NTVYLF | | | VYL | | | | | WB |
| | | | GNK | | | | | | | | |
| 304 | DRB1_ | 685 | KGFAVF | 2 | 1159 | FAVFNT | 0.967 | Sequence | 0.238348 | 3.31 | <= |
| | 1503 | | NTVYLF | | | VYL | | | | | WB |
| | | | GNK | | | | | | | | |
| 307 | DRB1_ | 686 | AVFNTV | 5 | 1160 | VYLFGN | 1 | Sequence | 0.492407 | 1.62 | <= |
| | 0402 | | YLFGNK | | | KSS | | | | | WB |
| | | | SSS | | | | | | | | |
| 307 | DRB1_ | 686 | AVFNTV | 5 | 1160 | VYLFGN | 0.993 | Sequence | 0.409526 | 2.28 | <= |
| | 1501 | | YLFGNK | | | KSS | | | | | WB |
| | | | SSS | | | | | | | | |
| 307 | DRB1_ | 686 | AVFNTV | 5 | 1160 | VYLFGN | 0.993 | Sequence | 0.251326 | 3.09 | <= |
| | 1503 | | YLFGNK | | | KSS | | | | | WB |
| | | | SSS | | | | | | | | |
| 308 | DRB1_ | 687 | VFNTVY | 4 | 1160 | VYLFGN | 1 | Sequence | 0.722722 | 0.46 | <= |
| | 0402 | | LFGNKS | | | KSS | | | | | SB |
| | | | SSE | | | | | | | | |
| 308 | DRB1_ | 687 | VFNTVY | 5 | 1161 | YLFGNK | 0.973 | Sequence | 0.399852 | 4.23 | <= |
| | 1101 | | LFGNKS | | | SSS | | | | | WB |
| | | | SSE | | | | | | | | |
| 308 | DRB1_ | 687 | VFNTVY | 4 | 1160 | VYLFGN | 1 | Sequence | 0.636588 | 1.05 | <= |
| | 1501 | | LFGNKS | | | KSS | | | | | WB |
| | | | SSE | | | | | | | | |
| 308 | DRB1_ | 687 | VFNTVY | 4 | 1160 | VYLFGN | 0.907 | Sequence | 0.322183 | 4.98 | <= |
| | 1601 | | LFGNKS | | | KSS | | | | | WB |
| | | | SSE | | | | | | | | |
| 308 | DRB1_ | 687 | VFNTVY | 4 | 1160 | VYLFGN | 1 | Sequence | 0.431156 | 1.31 | <= |
| | 1503 | | LFGNKS | | | KSS | | | | | WB |
| | | | SSE | | | | | | | | |
| 309 | DRB1_ | 688 | FNTVYL | 3 | 1160 | VYLFGN | 1 | Sequence | 0.819874 | 0.2 | <= |
| | 0402 | | FGNKSS | | | KSS | | | | | SB |
| | | | SED | | | | | | | | |
| 309 | DRB1_ | 688 | FNTVYL | 3 | 1160 | VYLFGN | 0.993 | Sequence | 0.437668 | 3.37 | <= |
| | 0404 | | FGNKSS | | | KSS | | | | | WB |
| | | | SED | | | | | | | | |
| 309 | DRB1_ | 688 | FNTVYL | 4 | 1161 | YLFGNK | 0.993 | Sequence | 0.64869 | 1.84 | <= |
| | 1101 | | FGNKSS | | | SSS | | | | | WB |
| | | | SED | | | | | | | | |
| 309 | DRB1_ | 688 | FNTVYL | 3 | 1160 | VYLFGN | 1 | Sequence | 0.763669 | 0.62 | <= |
| | 1501 | | FGNKSS | | | KSS | | | | | SB |
| | | | SED | | | | | | | | |
| 309 | DRB1_ | 688 | FNTVYL | 3 | 1160 | VYLFGN | 0.973 | Sequence | 0.434067 | 3.29 | <= |
| | 1601 | | FGNKSS | | | KSS | | | | | WB |
| | | | SED | | | | | | | | |
| 309 | DRB1_ | 688 | FNTVYL | 3 | 1160 | VYLFGN | 1 | Sequence | 0.562372 | 0.71 | <= |
| | 1503 | | FGNKSS | | | KSS | | | | | SB |
| | | | SED | | | | | | | | |
| 310 | DRB1_ | 689 | NTVYLF | 2 | 1160 | VYLFGN | 1 | Sequence | 0.708733 | 0.5 | <= |
| | 0402 | | GNKSSS | | | KSS | | | | | SB |
| | | | EDS | | | | | | | | |
| 310 | DRB1_ | 689 | NTVYLF | 3 | 1161 | YLFGNK | 0.6 | Sequence | 0.502361 | 3.53 | <= |
| | 0801 | | GNKSSS | | | SSS | | | | | WB |
| | | | EDS | | | | | | | | |
| 310 | DRB1_ | 689 | NTVYLF | 3 | 1161 | YLFGNK | 1 | Sequence | 0.707494 | 1.48 | <= |
| | 1101 | | GNKSSS | | | SSS | | | | | WB |
| | | | EDS | | | | | | | | |
| 310 | DRB1_ | 689 | NTVYLF | 2 | 1160 | VYLFGN | 1 | Sequence | 0.624009 | 1.1 | <= |
| | 1501 | | GNKSSS | | | KSS | | | | | WB |
| | | | EDS | | | | | | | | |
| 310 | DRB1_ | 689 | NTVYLF | 2 | 1160 | VYLFGN | 1 | Sequence | 0.422355 | 1.37 | <= |
| | 1503 | | GNKSSS | | | KSS | | | | | WB |
| | | | EDS | | | | | | | | |
| 310 | DRB1_ | 689 | NTVYLF | 3 | 1161 | YLFGNK | 0.713 | Sequence | 0.166963 | 4.39 | <= |
| | 1402 | | GNKSSS | | | SSS | | | | | WB |
| | | | EDS | | | | | | | | |
| 310 | DRB1_ | 689 | NTVYLF | 3 | 1161 | YLFGNK | 0.707 | Sequence | 0.345737 | 4.28 | <= |
| | 1303 | | GNKSSS | | | SSS | | | | | WB |
| | | | EDS | | | | | | | | |
| 310 | DRB1_ | 689 | NTVYLF | 3 | 1161 | YLFGNK | 0.42 | Sequence | 0.323362 | 3.8 | <= |
| | 0803 | | GNKSSS | | | SSS | | | | | WB |
| | | | EDS | | | | | | | | |
| 311 | DRB1_ | 690 | TVYLFG | 4 | 1162 | FGNKSS | 0.713 | Sequence | 0.493142 | 3.68 | <= |
| | 0801 | | NKSSSE | | | SED | | | | | WB |
| | | | DSS | | | | | | | | |
| 311 | DRB1_ | 690 | TVYLFG | 2 | 1161 | YLFGNK | 0.953 | Sequence | 0.388926 | 4.36 | <= |
| | 1101 | | NKSSSE | | | SSS | | | | | WB |
| | | | DSS | | | | | | | | |
| 311 | DRB1_ | 690 | TVYLFG | 4 | 1162 | FGNKSS | 0.573 | Sequence | 0.31277 | 4.09 | <= |
| | 0803 | | NKSSSE | | | SED | | | | | WB |
| | | | DSS | | | | | | | | |
| 312 | DRB1_ | 691 | VYLFGN | 3 | 1162 | FGNKSS | 0.9 | Sequence | 0.442965 | 4.59 | <= |
| | 0801 | | KSSSED | | | SED | | | | | WB |
| | | | SSF | | | | | | | | |
| 322 | DRB1_ | 692 | EDSSFL | 5 | 1163 | LDFDFN | 0.993 | Sequence | 0.224676 | 4.96 | <= |
| | 0301 | | DFDFNS | | | SVY | | | | | WB |
| | | | VYN | | | | | | | | |
| 323 | DRB1_ | 693 | DSSFLD | 4 | 1163 | LDFDFN | 1 | Sequence | 0.378421 | 2.99 | <= |
| | 0301 | | FDFNSV | | | SVY | | | | | WB |
| | | | YNS | | | | | | | | |
| 323 | DRB1_ | 693 | DSSFLD | 4 | 1163 | LDFDFN | 0.973 | Sequence | 0.116463 | 4.21 | <= |
| | 0305 | | FDFNSV | | | SVY | | | | | WB |
| | | | YNS | | | | | | | | |
| 324 | DRB1_ | 694 | SSFLDF | 3 | 1163 | LDFDFN | 1 | Sequence | 0.484094 | 2.15 | <= |
| | 0301 | | DFNSVY | | | SVY | | | | | WB |
| | | | NSG | | | | | | | | |
| 324 | DRB1_ | 694 | SSFLDF | 3 | 1163 | LDFDFN | 0.973 | Sequence | 0.157857 | 3.14 | <= |
| | 0305 | | DFNSVY | | | SVY | | | | | WB |
| | | | NSG | | | | | | | | |
| 325 | DRB1_ | 695 | SFLDFD | 2 | 1163 | LDFDFN | 0.98 | Sequence | 0.232444 | 4.82 | <= |
| | 0301 | | FNSVYN | | | SVY | | | | | WB |
| | | | SGK | | | | | | | | |
| 326 | DRB1_ | 696 | FLDFDF | 3 | 1164 | FDFNSV | 0.84 | Sequence | 0.162939 | 4.58 | <= |
| | 1402 | | NSVYNS | | | YNS | | | | | WB |
| | | | GKK | | | | | | | | |
| 328 | DRB1_ | 697 | DFDFNS | 3 | 1165 | FNSVYN | 0.98 | Sequence | 0.3529 | 4.42 | <= |
| | 1601 | | VYNSGK | | | SGK | | | | | WB |
| | | | KPY | | | | | | | | |
| 330 | DRB1_ | 698 | DFNSVY | 4 | 1166 | VYNSGK | 0.98 | Sequence | 0.609642 | 2.23 | <= |
| | 1301 | | NSGKKP | | | KPY | | | | | WB |
| | | | YIR | | | | | | | | |
| 330 | DRB1_ | 698 | DFNSVY | 4 | 1166 | VYNSGK | 0.707 | Sequence | 0.345551 | 4 | <= |
| | 1401 | | NSGKKP | | | KPY | | | | | WB |
| | | | YIR | | | | | | | | |
| 330 | DRB1_ | 698 | DFNSVY | 4 | 1166 | VYNSGK | 0.707 | Sequence | 0.345551 | 4 | <= |
| | 1454 | | NSGKKP | | | KPY | | | | | WB |
| | | | YIR | | | | | | | | |
| 331 | DRB1_ | 699 | FNSVYN | 3 | 1166 | VYNSGK | 0.927 | Sequence | 0.24145 | 4.67 | <= |
| | 0301 | | SGKKPY | | | KPY | | | | | WB |
| | | | IRN | | | | | | | | |
| 331 | DRB1_ | 699 | FNSVYN | 3 | 1166 | VYNSGK | 0.98 | Sequence | 0.691072 | 1.39 | <= |
| | 1301 | | SGKKPY | | | KPY | | | | | WB |
| | | | IRN | | | | | | | | |
| 331 | DRB1_ | 699 | FNSVYN | 3 | 1166 | VYNSGK | 0.687 | Sequence | 0.419062 | 2.66 | <= |
| | 1401 | | SGKKPY | | | KPY | | | | | WB |
| | | | IRN | | | | | | | | |
| 331 | DRB1_ | 699 | FNSVYN | 3 | 1166 | VYNSGK | 0.687 | Sequence | 0.419062 | 2.66 | <= |
| | 1454 | | SGKKPY | | | KPY | | | | | WB |
| | | | IRN | | | | | | | | |
| 331 | DRB1_ | 699 | FNSVYN | 4 | 1167 | YNSGKK | 0.72 | Sequence | 0.159816 | 4.76 | <= |
| | 1402 | | SGKKPY | | | PYI | | | | | WB |
| | | | IRN | | | | | | | | |
| 331 | DRB1_ | 699 | FNSVYN | 4 | 1167 | YNSGKK | 0.833 | Sequence | 0.320468 | 4.7 | <= |
| | 1303 | | SGKKPY | | | PYI | | | | | WB |
| | | | IRN | | | | | | | | |
| 331 | DRB1_ | 699 | FNSVYN | 3 | 1166 | VYNSGK | 0.64 | Sequence | 0.376079 | 2.74 | <= |
| | 1302 | | SGKKPY | | | KPY | | | | | WB |
| | | | IRN | | | | | | | | |
| 332 | DRB1_ | 700 | NSVYNS | 3 | 1167 | YNSGKK | 0.947 | Sequence | 0.39691 | 4.26 | <= |
| | 1101 | | GKKPYI | | | PYI | | | | | WB |
| | | | RNG | | | | | | | | |
| 332 | DRB1_ | 700 | NSVYNS | 2 | 1166 | VYNSGK | 0.913 | Sequence | 0.572595 | 2.65 | <= |
| | 1301 | | GKKPYI | | | KPY | | | | | WB |
| | | | RNG | | | | | | | | |
| 332 | DRB1_ | 700 | NSVYNS | 2 | 1166 | VYNSGK | 0.54 | Sequence | 0.356523 | 3.77 | <= |
| | 1401 | | GKKPYI | | | KPY | | | | | WB |
| | | | RNG | | | | | | | | |
| 332 | DRB1_ | 700 | NSVYNS | 2 | 1166 | VYNSGK | 0.54 | Sequence | 0.356523 | 3.77 | <= |
| | 1454 | | GKKPYI | | | KPY | | | | | WB |
| | | | RNG | | | | | | | | |
| 332 | DRB1_ | 700 | NSVYNS | 3 | 1167 | YNSGKK | 0.84 | Sequence | 0.185284 | 3.62 | <= |
| | 1402 | | GKKPYI | | | PYI | | | | | WB |
| | | | RNG | | | | | | | | |
| 332 | DRB1_ | 700 | NSVYNS | 3 | 1167 | YNSGKK | 0.893 | Sequence | 0.350557 | 4.2 | <= |
| | 1303 | | GKKPYI | | | PYI | | | | | WB |
| | | | RNG | | | | | | | | |
| 332 | DRB1_ | 700 | NSVYNS | 3 | 1167 | YNSGKK | 0.547 | Sequence | 0.334374 | 3.8 | <= |
| | 1302 | | GKKPYI | | | PYI | | | | | WB |
| | | | RNG | | | | | | | | |
| 339 | DRB1_ | 701 | KKPYIR | 3 | 1168 | YIRNGY | 0.94 | Sequence | 0.315117 | 4.37 | <= |
| | 1302 | | NGYLTY | | | LTY | | | | | WB |
| | | | FFA | | | | | | | | |
| 343 | DRB1_ | 702 | IRNGYL | 5 | 1169 | LTYFFA | 1 | Sequence | 0.321235 | 3.06 | <= |
| | 1501 | | TYFFAE | | | ENL | | | | | WB |
| | | | NLA | | | | | | | | |
| 343 | DRB1_ | 702 | IRNGYL | 5 | 1169 | LTYFFA | 0.987 | Sequence | 0.216232 | 3.74 | <= |
| | 1503 | | TYFFAE | | | ENL | | | | | WB |
| | | | NLA | | | | | | | | |
| 344 | DRB1_ | 703 | RNGYLT | 4 | 1169 | LTYFFA | 1 | Sequence | 0.393705 | 4.28 | <= |
| | 0102 | | YFFAEN | | | ENL | | | | | WB |
| | | | LAP | | | | | | | | |
| 344 | DRB1_ | 703 | RNGYLT | 4 | 1169 | LTYFFA | 0.98 | Sequence | 0.260761 | 4.55 | <= |
| | 0701 | | YFFAEN | | | ENL | | | | | WB |
| | | | LAP | | | | | | | | |
| 344 | DRB1_ | 703 | RNGYLT | 4 | 1169 | LTYFFA | 1 | Sequence | 0.571285 | 1.33 | <= |
| | 1501 | | YFFAEN | | | ENL | | | | | WB |
| | | | LAP | | | | | | | | |
| 344 | DRB1_ | 703 | RNGYLT | 4 | 1169 | LTYFFA | 0.993 | Sequence | 0.450891 | 3.08 | <= |
| | 1601 | | YFFAEN | | | ENL | | | | | WB |
| | | | LAP | | | | | | | | |
| 344 | DRB1_ | 703 | RNGYLT | 4 | 1169 | LTYFFA | 0.987 | Sequence | 0.445945 | 1.22 | <= |
| | 1503 | | YFFAEN | | | ENL | | | | | WB |
| | | | LAP | | | | | | | | |
| 345 | DRB1_ | 704 | NGYLTY | 3 | 1169 | LTYFFA | 0.987 | Sequence | 0.269427 | 4.95 | <= |
| | 0101 | | FFAENL | | | ENL | | | | | WB |
| | | | APS | | | | | | | | |
| 345 | DRB1_ | 704 | NGYLTY | 3 | 1169 | LTYFFA | 0.993 | Sequence | 0.491364 | 3.16 | <= |
| | 0102 | | FFAENL | | | ENL | | | | | WB |
| | | | APS | | | | | | | | |
| 345 | DRB1_ | 704 | NGYLTY | 3 | 1169 | LTYFFA | 0.967 | Sequence | 0.284398 | 4.05 | <= |
| | 0103 | | FFAENL | | | ENL | | | | | WB |
| | | | APS | | | | | | | | |
| 345 | DRB1_ | 704 | NGYLTY | 3 | 1169 | LTYFFA | 0.98 | Sequence | 0.301413 | 3.86 | <= |
| | 0701 | | FFAENL | | | ENL | | | | | WB |
| | | | APS | | | | | | | | |
| 345 | DRB1_ | 704 | NGYLTY | 3 | 1169 | LTYFFA | 0.86 | Sequence | 0.374577 | 4.41 | <= |
| | 0901 | | FFAENL | | | ENL | | | | | WB |
| | | | APS | | | | | | | | |
| 345 | DRB1_ | 704 | NGYLTY | 3 | 1169 | LTYFFA | 1 | Sequence | 0.649754 | 1 | <= |
| | 1501 | | FFAENL | | | ENL | | | | | SB |
| | | | APS | | | | | | | | |
| 345 | DRB1_ | 704 | NGYLTY | 3 | 1169 | LTYFFA | 0.993 | Sequence | 0.537953 | 2.11 | <= |
| | 1601 | | FFAENL | | | ENL | | | | | WB |
| | | | APS | | | | | | | | |
| 345 | DRB1_ | 704 | NGYLTY | 3 | 1169 | LTYFFA | 0.987 | Sequence | 0.531863 | 0.82 | <= |
| | 1503 | | FFAENL | | | ENL | | | | | SB |
| | | | APS | | | | | | | | |
| 346 | DRB1_ | 705 | GYLTYF | 2 | 1169 | LTYFFA | 0.98 | Sequence | 0.333623 | 2.93 | <= |
| | 1501 | | FAENLA | | | ENL | | | | | WB |
| | | | PSV | | | | | | | | |
| 346 | DRB1_ | 705 | GYLTYF | 2 | 1169 | LTYFFA | 0.907 | Sequence | 0.247481 | 3.15 | <= |
| | 1503 | | FAENLA | | | ENL | | | | | WB |
| | | | PSV | | | | | | | | |
| 347 | DRB1_ | 706 | YLTYFF | 3 | 1170 | YFFAEN | 0.533 | Sequence | 0.349474 | 3.71 | <= |
| | 0401 | | AENLAP | | | LAP | | | | | WB |
| | | | SVN | | | | | | | | |
| 347 | DRB1_ | 706 | YLTYFF | 4 | 1171 | FFAENL | 0.787 | Sequence | 0.526934 | 2.67 | <= |
| | 1001 | | AENLAP | | | APS | | | | | WB |
| | | | SVN | | | | | | | | |
| 347 | DRB1_ | 706 | YLTYFF | 3 | 1170 | YFFAEN | 0.733 | Sequence | 0.404843 | 3.62 | <= |
| | 0408 | | AENLAP | | | LAP | | | | | WB |
| | | | SVN | | | | | | | | |
| 348 | DRB1_ | 707 | LTYFFA | 3 | 1171 | FFAENL | 0.76 | Sequence | 0.373139 | 3.37 | <= |
| | 0401 | | ENLAPS | | | APS | | | | | WB |
| | | | VNK | | | | | | | | |
| 348 | DRB1_ | 707 | LTYFFA | 4 | 1172 | FAENLA | 0.733 | Sequence | 0.372851 | 4.45 | <= |
| | 0901 | | ENLAPS | | | PSV | | | | | WB |
| | | | VNK | | | | | | | | |
| 348 | DRB1_ | 707 | LTYFFA | 3 | 1171 | FFAENL | 0.8 | Sequence | 0.648466 | 1.49 | <= |
| | 1001 | | ENLAPS | | | APS | | | | | WB |
| | | | VNK | | | | | | | | |
| 348 | DRB1_ | 707 | LTYFFA | 4 | 1172 | FAENLA | 0.693 | Sequence | 0.244711 | 1.89 | <= |
| | 1402 | | ENLAPS | | | PSV | | | | | WB |
| | | | VNK | | | | | | | | |
| 348 | DRB1_ | 707 | LTYFFA | 4 | 1172 | FAENLA | 0.893 | Sequence | 0.402447 | 2.24 | <= |
| | 1302 | | ENLAPS | | | PSV | | | | | WB |
| | | | VNK | | | | | | | | |
| 348 | DRB1_ | 707 | LTYFFA | 3 | 1171 | FFAENL | 0.647 | Sequence | 0.360743 | 4.28 | <= |
| | 0408 | | ENLAPS | | | APS | | | | | WB |
| | | | VNK | | | | | | | | |
| 349 | DRB1_ | 708 | TYFFAE | 3 | 1172 | FAENLA | 0.88 | Sequence | 0.298285 | 4.52 | <= |
| | 0101 | | NLAPSV | | | PSV | | | | | WB |
| | | | NKD | | | | | | | | |
| 349 | DRB1_ | 708 | TYFFAE | 3 | 1172 | FAENLA | 0.853 | Sequence | 0.297788 | 3.83 | <= |
| | 0103 | | NLAPSV | | | PSV | | | | | WB |
| | | | NKD | | | | | | | | |
| 349 | DRB1_ | 708 | TYFFAE | 3 | 1172 | FAENLA | 0.813 | Sequence | 0.104153 | 4.66 | <= |
| | 0305 | | NLAPSV | | | PSV | | | | | WB |
| | | | NKD | | | | | | | | |
| 349 | DRB1_ | 708 | TYFFAE | 3 | 1172 | FAENLA | 0.94 | Sequence | 0.43192 | 3.28 | <= |
| | 0901 | | NLAPSV | | | PSV | | | | | WB |
| | | | NKD | | | | | | | | |
| 349 | DRB1_ | 708 | TYFFAE | 2 | 1171 | FFAENL | 0.687 | Sequence | 0.577294 | 2.14 | <= |
| | 1001 | | NLAPSV | | | APS | | | | | WB |
| | | | NKD | | | | | | | | |
| 349 | DRB1_ | 708 | TYFFAE | 3 | 1172 | FAENLA | 0.84 | Sequence | 0.285466 | 1.28 | <= |
| | 1402 | | NLAPSV | | | PSV | | | | | WB |
| | | | NKD | | | | | | | | |
| 349 | DRB1_ | 708 | TYFFAE | 3 | 1172 | FAENLA | 0.933 | Sequence | 0.4723 | 1.31 | <= |
| | 1302 | | NLAPSV | | | PSV | | | | | WB |
| | | | NKD | | | | | | | | |
| 350 | DRB1_ | 709 | YFFAEN | 2 | 1172 | FAENLA | 0.887 | Sequence | 0.313354 | 4.42 | <= |
| | 1302 | | LAPSVN | | | PSV | | | | | WB |
| | | | KDY | | | | | | | | |
| 356 | DRB1_ | 710 | LAPSVN | 4 | 1173 | VNKDYV | 1 | Sequence | 0.334875 | 3.39 | <= |
| | 0301 | | KDYVKL | | | KLI | | | | | WB |
| | | | IFD | | | | | | | | |
| 357 | DRB1_ | 711 | APSVNK | 3 | 1173 | VNKDYV | 1 | Sequence | 0.323593 | 3.49 | <= |
| | 0301 | | DYVKLI | | | KLI | | | | | WB |
| | | | FDI | | | | | | | | |
| 360 | DRB1_ | 712 | VNKDYV | 5 | 1174 | VKLIFD | 1 | Sequence | 0.63397 | 1.61 | <= |
| | 0404 | | KLIFDI | | | IYA | | | | | WB |
| | | | YAN | | | | | | | | |
| 361 | DRB1_ | 713 | NKDYVK | 4 | 1174 | VKLIFD | 1 | Sequence | 0.661226 | 1.42 | <= |
| | 0404 | | LIFDIY | | | IYA | | | | | WB |
| | | | ANL | | | | | | | | |
| 362 | DRB1_ | 714 | KDYVKL | 3 | 1174 | VKLIFD | 0.987 | Sequence | 0.337223 | 3.31 | <= |
| | 0402 | | IFDIYA | | | IYA | | | | | WB |
| | | | NLG | | | | | | | | |
| 362 | DRB1_ | 714 | KDYVKL | 3 | 1174 | VKLIFD | 1 | Sequence | 0.773602 | 0.79 | <= |
| | 0404 | | IFDIYA | | | IYA | | | | | SB |
| | | | NLG | | | | | | | | |
| 362 | DRB1_ | 714 | KDYVKL | 3 | 1174 | VKLIFD | 0.987 | Sequence | 0.229763 | 4.37 | <= |
| | 1501 | | IFDIYA | | | IYA | | | | | WB |
| | | | NLG | | | | | | | | |
| 362 | DRB1_ | 714 | KDYVKL | 3 | 1174 | VKLIFD | 0.987 | Sequence | 0.351443 | 4.43 | <= |
| | 0408 | | IFDIYA | | | IYA | | | | | WB |
| | | | NLG | | | | | | | | |
| 363 | DRB1_ | 715 | DYVKLI | 2 | 1174 | VKLIFD | 0.667 | Sequence | 0.401051 | 3.85 | <= |
| | 0404 | | FDIYAN | | | IYA | | | | | WB |
| | | | LGF | | | | | | | | |
| 364 | DRB1_ | 716 | YVKLIF | 4 | 1175 | IFDIYA | 0.933 | Sequence | 0.333056 | 4.89 | <= |
| | 0404 | | DIYANL | | | NLG | | | | | WB |
| | | | GFY | | | | | | | | |
| 364 | DRB1_ | 716 | YVKLIF | 5 | 1176 | FDIYAN | 0.767 | Sequence | 0.202974 | 4.92 | <= |
| | 1501 | | DIYANL | | | LGF | | | | | WB |
| | | | GFY | | | | | | | | |
| 365 | DRB1_ | 717 | VKLIFD | 3 | 1175 | IFDIYA | 0.987 | Sequence | 0.349592 | 4.92 | <= |
| | 0102 | | IYANLG | | | NLG | | | | | WB |
| | | | FYS | | | | | | | | |
| 365 | DRB1_ | 717 | VKLIFD | 3 | 1175 | IFDIYA | 0.967 | Sequence | 0.415748 | 3.65 | <= |
| | 0404 | | IYANLG | | | NLG | | | | | WB |
| | | | FYS | | | | | | | | |
| 365 | DRB1_ | 717 | VKLIFD | 4 | 1176 | FDIYAN | 0.82 | Sequence | 0.338606 | 2.89 | <= |
| | 1501 | | IYANLG | | | LGF | | | | | WB |
| | | | FYS | | | | | | | | |
| 365 | DRB1_ | 717 | VKLIFD | 4 | 1176 | FDIYAN | 0.773 | Sequence | 0.216558 | 3.73 | <= |
| | 1503 | | IYANLG | | | LGF | | | | | WB |
| | | | FYS | | | | | | | | |
| 366 | DRB1_ | 718 | KLIFDI | 3 | 1176 | FDIYAN | 0.927 | Sequence | 0.436273 | 2.08 | <= |
| | 1501 | | YANLGF | | | LGF | | | | | WB |
| | | | YSG | | | | | | | | |
| 366 | DRB1_ | 718 | KLIFDI | 3 | 1176 | FDIYAN | 0.84 | Sequence | 0.282573 | 2.64 | <= |
| | 1503 | | YANLGF | | | LGF | | | | | WB |
| | | | YSG | | | | | | | | |
| 372 | DRB1_ | 719 | YANLGF | 3 | 1177 | LGFYSG | 0.98 | Sequence | 0.275981 | 3.61 | <= |
| | 1501 | | YSGIDF | | | IDF | | | | | WB |
| | | | NLG | | | | | | | | |
| 372 | DRB1_ | 719 | YANLGF | 3 | 1177 | LGFYSG | 0.92 | Sequence | 0.189985 | 4.38 | <= |
| | 1503 | | YSGIDF | | | IDF | | | | | WB |
| | | | NLG | | | | | | | | |
| 376 | DRB1_ | 720 | GFYSGI | 5 | 1178 | IDFNLG | 0.993 | Sequence | 0.329004 | 3.96 | <= |
| | 1302 | | DENLGN | | | NTL | | | | | WB |
| | | | TLG | | | | | | | | |
| 377 | DRB1_ | 721 | FYSGID | 4 | 1178 | IDFNLG | 1 | Sequence | 0.473434 | 1.3 | <= |
| | 1302 | | FNLGNT | | | NTL | | | | | WB |
| | | | LGH | | | | | | | | |
| 378 | DRB1_ | 722 | YSGIDE | 3 | 1178 | IDFNLG | 0.993 | Sequence | 0.452979 | 1.53 | <= |
| | 1302 | | NLGNTL | | | NTL | | | | | WB |
| | | | GHF | | | | | | | | |
| 379 | DRB1_ | 723 | SGIDFN | 2 | 1178 | IDFNLG | 0.92 | Sequence | 0.308766 | 4.57 | <= |
| | 1302 | | LGNTLG | | | NTL | | | | | WB |
| | | | HFK | | | | | | | | |
| 384 | DRB1_ | 724 | NLGNTL | 5 | 1179 | LGHFKT | 1 | Sequence | 0.66759 | 0.66 | <= |
| | 0402 | | GHFKTL | | | LEG | | | | | SB |
| | | | EGN | | | | | | | | |
| 384 | DRB1_ | 724 | NLGNTL | 5 | 1179 | LGHFKT | 1 | Sequence | 0.498052 | 2.72 | <= |
| | 0404 | | GHFKTL | | | LEG | | | | | WB |
| | | | EGN | | | | | | | | |
| 385 | DRB1_ | 725 | LGNTLG | 4 | 1179 | LGHFKT | 1 | Sequence | 0.784453 | 0.28 | <= |
| | 0402 | | HFKTLE | | | LEG | | | | | SB |
| | | | GNF | | | | | | | | |
| 385 | DRB1_ | 725 | LGNTLG | 4 | 1179 | LGHFKT | 1 | Sequence | 0.636325 | 1.59 | <= |
| | 0404 | | HFKTLE | | | LEG | | | | | WB |
| | | | GNF | | | | | | | | |
| 385 | DRB1_ | 725 | LGNTLG | 4 | 1179 | LGHFKT | 1 | Sequence | 0.254652 | 3.91 | <= |
| | 1501 | | HFKTLE | | | LEG | | | | | WB |
| | | | GNF | | | | | | | | |
| 385 | DRB1_ | 725 | LGNTLG | 4 | 1179 | LGHFKT | 1 | Sequence | 0.177398 | 4.75 | <= |
| | 1503 | | HFKTLE | | | LEG | | | | | WB |
| | | | GNF | | | | | | | | |
| 386 | DRB1_ | 726 | GNTLGH | 3 | 1179 | LGHFKT | 1 | Sequence | 0.844278 | 0.16 | <= |
| | 0402 | | FKTLEG | | | LEG | | | | | SB |
| | | | NFG | | | | | | | | |
| 386 | DRB1_ | 726 | GNTLGH | 3 | 1179 | LGHFKT | 1 | Sequence | 0.725504 | 1.03 | <= |
| | 0404 | | FKTLEG | | | LEG | | | | | WB |
| | | | NFG | | | | | | | | |
| 386 | DRB1_ | 726 | GNTLGH | 3 | 1179 | LGHFKT | 0.993 | Sequence | 0.338401 | 2.89 | <= |
| | 1501 | | FKTLEG | | | LEG | | | | | WB |
| | | | NFG | | | | | | | | |
| 386 | DRB1_ | 726 | GNTLGH | 3 | 1179 | LGHFKT | 0.973 | Sequence | 0.2372 | 3.33 | <= |
| | 1503 | | FKTLEG | | | LEG | | | | | WB |
| | | | NFG | | | | | | | | |
| 386 | DRB1_ | 726 | GNTLGH | 3 | 1179 | LGHFKT | 0.833 | Sequence | 0.386707 | 3.87 | <= |
| | 0408 | | FKTLEG | | | LEG | | | | | WB |
| | | | NFG | | | | | | | | |
| 387 | DRB1_ | 727 | NTLGHF | 5 | 1180 | FKTLEG | 1 | Sequence | 0.626055 | 1.73 | <= |
| | 0101 | | KTLEGN | | | NFG | | | | | WB |
| | | | FGL | | | | | | | | |
| 387 | DRB1_ | 727 | NTLGHF | 5 | 1180 | FKTLEG | 0.967 | Sequence | 0.374998 | 4.52 | <= |
| | 0102 | | KTLEGN | | | NFG | | | | | WB |
| | | | FGL | | | | | | | | |
| 387 | DRB1_ | 727 | NTLGHF | 2 | 1179 | LGHFKT | 0.993 | Sequence | 0.601359 | 0.96 | <= |
| | 0402 | | KTLEGN | | | LEG | | | | | SB |
| | | | FGL | | | | | | | | |
| 387 | DRB1_ | 727 | NTLGHF | 2 | 1179 | LGHFKT | 0.86 | Sequence | 0.410384 | 3.72 | <= |
| | 0404 | | KTLEGN | | | LEG | | | | | WB |
| | | | FGL | | | | | | | | |
| 387 | DRB1_ | 727 | NTLGHF | 5 | 1180 | FKTLEG | 0.973 | Sequence | 0.553203 | 2.38 | <= |
| | 1001 | | KTLEGN | | | NFG | | | | | WB |
| | | | FGL | | | | | | | | |
| 387 | DRB1_ | 727 | NTLGHF | 5 | 1180 | FKTLEG | 0.8 | Sequence | 0.342168 | 4.59 | <= |
| | 0408 | | KTLEGN | | | NFG | | | | | WB |
| | | | FGL | | | | | | | | |
| 388 | DRB1_ | 728 | TLGHFK | 4 | 1180 | FKTLEG | 1 | Sequence | 0.876977 | 0.52 | <= |
| | 0101 | | TLEGNF | | | NFG | | | | | SB |
| | | | GLG | | | | | | | | |
| 388 | DRB1_ | 728 | TLGHFK | 4 | 1180 | FKTLEG | 1 | Sequence | 0.649599 | 1.79 | <= |
| | 0102 | | TLEGNF | | | NFG | | | | | WB |
| | | | GLG | | | | | | | | |
| 388 | DRB1_ | 728 | TLGHFK | 4 | 1180 | FKTLEG | 0.973 | Sequence | 0.300513 | 3.78 | <= |
| | 0103 | | TLEGNF | | | NFG | | | | | WB |
| | | | GLG | | | | | | | | |
| 388 | DRB1_ | 728 | TLGHFK | 4 | 1180 | FKTLEG | 1 | Sequence | 0.353822 | 3.64 | <= |
| | 0401 | | TLEGNF | | | NFG | | | | | WB |
| | | | GLG | | | | | | | | |
| 388 | DRB1_ | 728 | TLGHFK | 4 | 1180 | FKTLEG | 1 | Sequence | 0.722354 | 0.96 | <= |
| | 1001 | | TLEGNF | | | NFG | | | | | SB |
| | | | GLG | | | | | | | | |
| 388 | DRB1_ | 728 | TLGHFK | 4 | 1180 | FKTLEG | 0.993 | Sequence | 0.482619 | 2.7 | <= |
| | 1601 | | TLEGNF | | | NFG | | | | | WB |
| | | | GLG | | | | | | | | |
| 388 | DRB1_ | 728 | TLGHFK | 4 | 1180 | FKTLEG | 1 | Sequence | 0.525764 | 2.31 | <= |
| | 0408 | | TLEGNF | | | NFG | | | | | WB |
| | | | GLG | | | | | | | | |
| 389 | DRB1_ | 729 | LGHFKT | 3 | 1180 | FKTLEG | 1 | Sequence | 0.871984 | 0.54 | <= |
| | 0101 | | LEGNFG | | | NFG | | | | | SB |
| | | | LGF | | | | | | | | |
| 389 | DRB1_ | 729 | LGHFKT | 3 | 1180 | FKTLEG | 1 | Sequence | 0.641439 | 1.85 | <= |
| | 0102 | | LEGNFG | | | NFG | | | | | WB |
| | | | LGF | | | | | | | | |
| 389 | DRB1_ | 729 | LGHFKT | 3 | 1180 | FKTLEG | 0.987 | Sequence | 0.282027 | 4.09 | <= |
| | 0103 | | LEGNFG | | | NFG | | | | | WB |
| | | | LGF | | | | | | | | |
| 389 | DRB1_ | 729 | LGHFKT | 3 | 1180 | FKTLEG | 1 | Sequence | 0.318197 | 4.21 | <= |
| | 0401 | | LEGNFG | | | NFG | | | | | WB |
| | | | LGF | | | | | | | | |
| 389 | DRB1_ | 729 | LGHFKT | 3 | 1180 | FKTLEG | 1 | Sequence | 0.705761 | 1.06 | <= |
| | 1001 | | LEGNFG | | | NFG | | | | | WB |
| | | | LGF | | | | | | | | |
| 389 | DRB1_ | 729 | LGHFKT | 3 | 1180 | FKTLEG | 1 | Sequence | 0.456527 | 3.01 | <= |
| | 1601 | | LEGNFG | | | NFG | | | | | WB |
| | | | LGF | | | | | | | | |
| 389 | DRB1_ | 729 | LGHFKT | 3 | 1180 | FKTLEG | 1 | Sequence | 0.49536 | 2.61 | <= |
| | 0408 | | LEGNFG | | | NFG | | | | | WB |
| | | | LGF | | | | | | | | |
| 390 | DRB1_ | 730 | GHFKTL | 2 | 1180 | FKTLEG | 1 | Sequence | 0.753465 | 1.1 | <= |
| | 0101 | | EGNFGL | | | NFG | | | | | WB |
| | | | GFT | | | | | | | | |
| 390 | DRB1_ | 730 | GHFKTL | 2 | 1180 | FKTLEG | 1 | Sequence | 0.503668 | 3.03 | <= |
| | 0102 | | EGNFGL | | | NFG | | | | | WB |
| | | | GFT | | | | | | | | |
| 390 | DRB1_ | 730 | GHFKTL | 2 | 1180 | FKTLEG | 1 | Sequence | 0.586849 | 2.05 | <= |
| | 1001 | | EGNFGL | | | NFG | | | | | WB |
| | | | GFT | | | | | | | | |
| 398 | DRB1_ | 731 | NFGLGF | 3 | 1181 | LGFTRN | 1 | Sequence | 0.289255 | 4.15 | <= |
| | 0402 | | TRNIYS | | | IYS | | | | | WB |
| | | | YDG | | | | | | | | |
| 398 | DRB1_ | 731 | NFGLGF | 3 | 1181 | LGFTRN | 1 | Sequence | 0.34565 | 4.66 | <= |
| | 0404 | | TRNIYS | | | IYS | | | | | WB |
| | | | YDG | | | | | | | | |
| 401 | DRB1_ | 732 | LGFTRN | 6 | 1182 | IYSYDG | 0.98 | Sequence | 0.220968 | 4.55 | <= |
| | 1501 | | IYSYDG | | | GYY | | | | | WB |
| | | | GYY | | | | | | | | |
| 401 | DRB1_ | 732 | LGFTRN | 6 | 1182 | IYSYDG | 0.98 | Sequence | 0.201922 | 4.05 | <= |
| | 1503 | | IYSYDG | | | GYY | | | | | WB |
| | | | GYY | | | | | | | | |
| 402 | DRB1_ | 733 | GFTRNI | 5 | 1182 | IYSYDG | 1 | Sequence | 0.642368 | 1.03 | <= |
| | 1501 | | YSYDGG | | | GYY | | | | | WB |
| | | | YYP | | | | | | | | |
| 402 | DRB1_ | 733 | GFTRNI | 5 | 1182 | IYSYDG | 0.987 | Sequence | 0.37642 | 4.05 | <= |
| | 1601 | | YSYDGG | | | GYY | | | | | WB |
| | | | YYP | | | | | | | | |
| 402 | DRB1_ | 733 | GFTRNI | 5 | 1182 | IYSYDG | 0.993 | Sequence | 0.567514 | 0.69 | <= |
| | 1503 | | YSYDGG | | | GYY | | | | | SB |
| | | | YYP | | | | | | | | |
| 403 | DRB1_ | 734 | FTRNIY | 4 | 1182 | IYSYDG | 0.993 | Sequence | 0.276398 | 4.83 | <= |
| | 1201 | | SYDGGY | | | GYY | | | | | WB |
| | | | YPF | | | | | | | | |
| 403 | DRB1_ | 734 | FTRNIY | 4 | 1182 | IYSYDG | 1 | Sequence | 0.707843 | 0.78 | <= |
| | 1501 | | SYDGGY | | | GYY | | | | | SB |
| | | | YPF | | | | | | | | |
| 403 | DRB1_ | 734 | FTRNIY | 4 | 1182 | IYSYDG | 0.993 | Sequence | 0.469498 | 2.86 | <= |
| | 1601 | | SYDGGY | | | GYY | | | | | WB |
| | | | YPF | | | | | | | | |
| 403 | DRB1_ | 734 | FTRNIY | 4 | 1182 | IYSYDG | 0.993 | Sequence | 0.625062 | 0.51 | <= |
| | 1503 | | SYDGGY | | | GYY | | | | | SB |
| | | | YPF | | | | | | | | |
| 404 | DRB1_ | 735 | TRNIYS | 3 | 1182 | IYSYDG | 0.98 | Sequence | 0.350194 | 3.14 | <= |
| | 1201 | | YDGGYY | | | GYY | | | | | WB |
| | | | PFD | | | | | | | | |
| 404 | DRB1_ | 735 | TRNIYS | 3 | 1182 | IYSYDG | 0.987 | Sequence | 0.467961 | 4.22 | <= |
| | 1301 | | YDGGYY | | | GYY | | | | | WB |
| | | | PFD | | | | | | | | |
| 404 | DRB1_ | 735 | TRNIYS | 3 | 1182 | IYSYDG | 1 | Sequence | 0.789412 | 0.54 | <= |
| | 1501 | | YDGGYY | | | GYY | | | | | SB |
| | | | PFD | | | | | | | | |
| 404 | DRB1_ | 735 | TRNIYS | 3 | 1182 | IYSYDG | 0.987 | Sequence | 0.572332 | 1.83 | <= |
| | 1601 | | YDGGYY | | | GYY | | | | | WB |
| | | | PFD | | | | | | | | |
| 404 | DRB1_ | 735 | TRNIYS | 3 | 1182 | IYSYDG | 1 | Sequence | 0.709848 | 0.31 | <= |
| | 1503 | | YDGGYY | | | GYY | | | | | SB |
| | | | PFD | | | | | | | | |
| 405 | DRB1_ | 736 | RNIYSY | 3 | 1183 | YSYDGG | 0.9 | Sequence | 0.105766 | 4.59 | <= |
| | 0305 | | DGGYYP | | | YYP | | | | | WB |
| | | | FDN | | | | | | | | |
| 405 | DRB1_ | 736 | RNIYSY | 2 | 1182 | IYSYDG | 1 | Sequence | 0.602543 | 1.19 | <= |
| | 1501 | | DGGYYP | | | GYY | | | | | WB |
| | | | FDN | | | | | | | | |
| 405 | DRB1_ | 736 | RNIYSY | 2 | 1182 | IYSYDG | 0.933 | Sequence | 0.361281 | 4.29 | <= |
| | 1601 | | DGGYYP | | | GYY | | | | | WB |
| | | | FDN | | | | | | | | |
| 405 | DRB1_ | 736 | RNIYSY | 2 | 1182 | IYSYDG | 1 | Sequence | 0.520818 | 0.86 | <= |
| | 1503 | | DGGYYP | | | GYY | | | | | SB |
| | | | FDN | | | | | | | | |
| 408 | DRB1_ | 737 | YSYDGG | 6 | 1184 | YYPFDN | 1 | Sequence | 0.247781 | 4.01 | <= |
| | 1501 | | YYPFDN | | | RTL | | | | | WB |
| | | | RTL | | | | | | | | |
| 408 | DRB1_ | 737 | YSYDGG | 6 | 1184 | YYPFDN | 1 | Sequence | 0.377103 | 4.04 | <= |
| | 1601 | | YYPFDN | | | RTL | | | | | WB |
| | | | RTL | | | | | | | | |
| 409 | DRB1_ | 738 | SYDGGY | 5 | 1184 | YYPFDN | 1 | Sequence | 0.541324 | 1.33 | <= |
| | 0103 | | YPFDNR | | | RTL | | | | | WB |
| | | | TLK | | | | | | | | |
| 409 | DRB1_ | 738 | SYDGGY | 5 | 1184 | YYPFDN | 1 | Sequence | 0.362548 | 3 | <= |
| | 0701 | | YPFDNR | | | RTL | | | | | WB |
| | | | TLK | | | | | | | | |
| 409 | DRB1_ | 738 | SYDGGY | 5 | 1184 | YYPFDN | 0.987 | Sequence | 0.357383 | 4.84 | <= |
| | 0901 | | YPFDNR | | | RTL | | | | | WB |
| | | | TLK | | | | | | | | |
| 409 | DRB1_ | 738 | SYDGGY | 5 | 1184 | YYPFDN | 1 | Sequence | 0.646349 | 1.01 | <= |
| | 1501 | | YPFDNR | | | RTL | | | | | WB |
| | | | TLK | | | | | | | | |
| 409 | DRB1_ | 738 | SYDGGY | 5 | 1184 | YYPFDN | 1 | Sequence | 0.845398 | 0.29 | <= |
| | 1601 | | YPFDNR | | | RTL | | | | | SB |
| | | | TLK | | | | | | | | |
| 409 | DRB1_ | 738 | SYDGGY | 5 | 1184 | YYPFDN | 1 | Sequence | 0.497861 | 0.96 | <= |
| | 1503 | | YPFDNR | | | RTL | | | | | SB |
| | | | TLK | | | | | | | | |
| 409 | DRB1_ | 738 | SYDGGY | 5 | 1184 | YYPFDN | 0.973 | Sequence | 0.300717 | 4.45 | <= |
| | 0803 | | YPFDNR | | | RTL | | | | | WB |
| | | | TLK | | | | | | | | |
| 410 | DRB1_ | 739 | YDGGYY | 4 | 1184 | YYPFDN | 1 | Sequence | 0.300953 | 4.48 | <= |
| | 0101 | | PFDNRT | | | RTL | | | | | WB |
| | | | LKQ | | | | | | | | |
| 410 | DRB1_ | 739 | YDGGYY | 4 | 1184 | YYPFDN | 1 | Sequence | 0.696038 | 0.63 | <= |
| | 0103 | | PFDNRT | | | RTL | | | | | SB |
| | | | LKQ | | | | | | | | |
| 410 | DRB1_ | 739 | YDGGYY | 4 | 1184 | YYPFDN | 0.993 | Sequence | 0.321605 | 3.56 | <= |
| | 0402 | | PFDNRT | | | RTL | | | | | WB |
| | | | LKQ | | | | | | | | |
| 410 | DRB1_ | 739 | YDGGYY | 4 | 1184 | YYPFDN | 0.993 | Sequence | 0.348331 | 3.27 | <= |
| | 0405 | | PFDNRT | | | RTL | | | | | WB |
| | | | LKQ | | | | | | | | |
| 410 | DRB1_ | 739 | YDGGYY | 4 | 1184 | YYPFDN | 1 | Sequence | 0.464843 | 2.04 | <= |
| | 0701 | | PFDNRT | | | RTL | | | | | WB |
| | | | LKQ | | | | | | | | |
| 410 | DRB1_ | 739 | YDGGYY | 4 | 1184 | YYPFDN | 1 | Sequence | 0.461197 | 2.79 | <= |
| | 0901 | | PFDNRT | | | RTL | | | | | WB |
| | | | LKQ | | | | | | | | |
| 410 | DRB1_ | 739 | YDGGYY | 4 | 1184 | YYPFDN | 1 | Sequence | 0.756775 | 0.64 | <= |
| | 1501 | | PFDNRT | | | RTL | | | | | SB |
| | | | LKQ | | | | | | | | |
| 410 | DRB1_ | 739 | YDGGYY | 4 | 1184 | YYPFDN | 1 | Sequence | 0.921337 | 0.09 | <= |
| | 1601 | | PFDNRT | | | RTL | | | | | SB |
| | | | LKQ | | | | | | | | |
| 410 | DRB1_ | 739 | YDGGYY | 4 | 1184 | YYPFDN | 1 | Sequence | 0.629314 | 0.5 | <= |
| | 1503 | | PFDNRT | | | RTL | | | | | SB |
| | | | LKQ | | | | | | | | |
| 410 | DRB1_ | 739 | YDGGYY | 4 | 1184 | YYPFDN | 0.98 | Sequence | 0.390068 | 2.41 | <= |
| | 0803 | | PFDNRT | | | RTL | | | | | WB |
| | | | LKQ | | | | | | | | |
| 411 | DRB1_ | 740 | DGGYYP | 3 | 1184 | YYPFDN | 1 | Sequence | 0.362366 | 3.79 | <= |
| | 0101 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.745809 | 0.49 | <= |
| | 0103 | | FDNRTL | | | RTL | | | | | SB |
| | | | KQS | | | | | | | | |
| 411 | DRB1_ | 740 | DGGYYP | 3 | 1184 | YYPFDN | 0.993 | Sequence | 0.361079 | 2.96 | <= |
| | 0402 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.375558 | 2.94 | <= |
| | 0405 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.495649 | 1.82 | <= |
| | 0701 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.492938 | 2.34 | <= |
| | 0901 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.785185 | 0.56 | <= |
| | 1501 | | FDNRTL | | | RTL | | | | | SB |
| | | | KQS | | | | | | | | |
| 411 | DRB1_ | 740 | DGGYYP | 3 | 1184 | YYPFDN | 1 | Sequence | 0.935814 | 0.05 | <= |
| | 1601 | | FDNRTL | | | RTL | | | | | SB |
| | | | KQS | | | | | | | | |
| 411 | DRB1_ | 740 | DGGYYP | 3 | 1184 | YYPFDN | 1 | Sequence | 0.670984 | 0.4 | <= |
| | 1503 | | FDNRTL | | | RTL | | | | | SB |
| | | | KQS | | | | | | | | |
| 411 | DRB1_ | 740 | DGGYYP | 3 | 1184 | YYPFDN | 0.953 | Sequence | 0.307057 | 4.63 | <= |
| | 1302 | | FDNRTL | | | RTL | | | | | WB |
| | | | KQS | | | | | | | | |
| 411 | DRB1_ | 740 | DGGYYP | 3 | 1184 | YYPFDN | 0.94 | Sequence | 0.421415 | 1.94 | <= |
| | 0803 | | FDNRTL | | | RTL | | | | | WB |
| | | | KQS | | | | | | | | |
| 411 | DRB1_ | 740 | DGGYYP | 3 | 1184 | YYPFDN | 1 | Sequence | 0.357756 | 4.33 | <= |
| | 0408 | | FDNRTL | | | RTL | | | | | WB |
| | | | KQS | | | | | | | | |
| 412 | DRB1_ | 741 | GGYYPF | 2 | 1184 | YYPFDN | 0.993 | Sequence | 0.547156 | 1.3 | <= |
| | 0103 | | DNRTLK | | | RTL | | | | | WB |
| | | | QSL | | | | | | | | |
| 412 | DRB1_ | 741 | GGYYPF | 2 | 1184 | YYPFDN | 0.993 | Sequence | 0.263165 | 4.5 | <= |
| | 0701 | | DNRTLK | | | RTL | | | | | WB |
| | | | QSL | | | | | | | | |
| 412 | DRB1_ | 741 | GGYYPF | 2 | 1184 | YYPFDN | 1 | Sequence | 0.601337 | 1.2 | <= |
| | 1501 | | DNRTLK | | | RTL | | | | | WB |
| | | | QSL | | | | | | | | |
| 412 | DRB1_ | 741 | GGYYPF | 2 | 1184 | YYPFDN | 1 | Sequence | 0.830324 | 0.34 | <= |
| | 1601 | | DNRTLK | | | RTL | | | | | SB |
| | | | QSL | | | | | | | | |
| 412 | DRB1_ | 741 | GGYYPF | 2 | 1184 | YYPFDN | 1 | Sequence | 0.473773 | 1.09 | <= |
| | 1503 | | DNRTLK | | | RTL | | | | | WB |
| | | | QSL | | | | | | | | |
| 413 | DRB1_ | 742 | GYYPFD | 1 | 1184 | YYPFDN | 1 | Sequence | 0.389117 | 3.87 | <= |
| | 1601 | | NRTLKQ | | | RTL | | | | | WB |
| | | | SLF | | | | | | | | |
| 421 | DRB1_ | 743 | TLKQSL | 5 | 1185 | LFSFSN | 1 | Sequence | 0.271264 | 3.67 | <= |
| | 1501 | | FSFSNL | | | LNK | | | | | WB |
| | | | NKG | | | | | | | | |
| 421 | DRB1_ | 743 | TLKQSL | 5 | 1185 | LFSFSN | 1 | Sequence | 0.397045 | 3.77 | <= |
| | 1601 | | FSFSNL | | | LNK | | | | | WB |
| | | | NKG | | | | | | | | |
| 421 | DRB1_ | 743 | TLKQSL | 5 | 1185 | LFSFSN | 0.987 | Sequence | 0.246984 | 3.16 | <= |
| | 1503 | | FSFSNL | | | LNK | | | | | WB |
| | | | NKG | | | | | | | | |
| 422 | DRB1_ | 744 | LKQSLF | 4 | 1185 | LFSFSN | 0.927 | Sequence | 0.401566 | 2.46 | <= |
| | 0402 | | SFSNLN | | | LNK | | | | | WB |
| | | | KGD | | | | | | | | |
| 422 | DRB1_ | 744 | LKQSLF | 4 | 1185 | LFSFSN | 1 | Sequence | 0.470403 | 1.85 | <= |
| | 1501 | | SFSNLN | | | LNK | | | | | WB |
| | | | KGD | | | | | | | | |
| 422 | DRB1_ | 744 | LKQSLF | 4 | 1185 | LFSFSN | 1 | Sequence | 0.61331 | 1.5 | <= |
| | 1601 | | SFSNLN | | | LNK | | | | | WB |
| | | | KGD | | | | | | | | |
| 422 | DRB1_ | 744 | LKQSLF | 4 | 1185 | LFSFSN | 1 | Sequence | 0.429871 | 1.32 | <= |
| | 1503 | | SFSNLN | | | LNK | | | | | WB |
| | | | KGD | | | | | | | | |
| 423 | DRB1_ | 745 | KQSLFS | 3 | 1185 | LFSFSN | 0.893 | Sequence | 0.412214 | 2.35 | <= |
| | 0402 | | FSNLNK | | | LNK | | | | | WB |
| | | | GDV | | | | | | | | |
| 423 | DRB1_ | 745 | KQSLFS | 3 | 1185 | LFSFSN | 0.96 | Sequence | 0.432452 | 4.89 | <= |
| | 1301 | | FSNLNK | | | LNK | | | | | WB |
| | | | GDV | | | | | | | | |
| 423 | DRB1_ | 745 | KQSLFS | 3 | 1185 | LFSFSN | 1 | Sequence | 0.487692 | 1.75 | <= |
| | 1501 | | FSNLNK | | | LNK | | | | | WB |
| | | | GDV | | | | | | | | |
| 423 | DRB1_ | 745 | KQSLFS | 3 | 1185 | LFSFSN | 1 | Sequence | 0.627991 | 1.39 | <= |
| | 1601 | | FSNLNK | | | LNK | | | | | WB |
| | | | GDV | | | | | | | | |
| 423 | DRB1_ | 745 | KQSLFS | 3 | 1185 | LFSFSN | 1 | Sequence | 0.446893 | 1.22 | <= |
| | 1503 | | FSNLNK | | | LNK | | | | | WB |
| | | | GDV | | | | | | | | |
| 424 | DRB1_ | 746 | QSLFSF | 2 | 1185 | LFSFSN | 0.993 | Sequence | 0.21643 | 4.64 | <= |
| | 1501 | | SNLNKG | | | LNK | | | | | WB |
| | | | DVF | | | | | | | | |
| 424 | DRB1_ | 746 | QSLFSF | 2 | 1185 | LFSFSN | 0.953 | Sequence | 0.326771 | 4.9 | <= |
| | 1601 | | SNLNKG | | | LNK | | | | | WB |
| | | | DVF | | | | | | | | |
| 424 | DRB1_ | 746 | QSLFSF | 2 | 1185 | LFSFSN | 0.987 | Sequence | 0.180064 | 4.67 | <= |
| | 1503 | | SNLNKG | | | LNK | | | | | WB |
| | | | DVF | | | | | | | | |
| 438 | DRB1_ | 747 | FGFEVP | 6 | 1186 | FRYLLK | 1 | Sequence | 0.351914 | 4.91 | <= |
| | 1101 | | FRYLLK | | | FKT | | | | | WB |
| | | | FKT | | | | | | | | |
| 439 | DRB1_ | 748 | GFEVPF | 5 | 1186 | FRYLLK | 1 | Sequence | 0.659872 | 1.64 | <= |
| | 0801 | | RYLLKF | | | FKT | | | | | WB |
| | | | KTE | | | | | | | | |
| 439 | DRB1_ | 748 | GFEVPF | 5 | 1186 | FRYLLK | 1 | Sequence | 0.861588 | 0.63 | <= |
| | 1101 | | RYLLKF | | | FKT | | | | | SB |
| | | | KTE | | | | | | | | |
| 439 | DRB1_ | 748 | GFEVPF | 5 | 1186 | FRYLLK | 1 | Sequence | 0.548186 | 2.9 | <= |
| | 1104 | | RYLLKF | | | FKT | | | | | WB |
| | | | KTE | | | | | | | | |
| 439 | DRB1_ | 748 | GFEVPF | 5 | 1186 | FRYLLK | 1 | Sequence | 0.197284 | 3.19 | <= |
| | 1402 | | RYLLKF | | | FKT | | | | | WB |
| | | | KTE | | | | | | | | |
| 439 | DRB1_ | 748 | GFEVPF | 5 | 1186 | FRYLLK | 1 | Sequence | 0.376503 | 3.82 | <= |
| | 1303 | | RYLLKF | | | FKT | | | | | WB |
| | | | KTE | | | | | | | | |
| 439 | DRB1_ | 748 | GFEVPF | 5 | 1186 | FRYLLK | 0.993 | Sequence | 0.446414 | 1.61 | <= |
| | 0803 | | RYLLKF | | | FKT | | | | | WB |
| | | | KTE | | | | | | | | |
| 440 | DRB1_ | 749 | FEVPFR | 4 | 1186 | FRYLLK | 1 | Sequence | 0.670543 | 1.55 | <= |
| | 0801 | | YLLKFK | | | FKT | | | | | WB |
| | | | TEF | | | | | | | | |
| 440 | DRB1_ | 749 | FEVPFR | 4 | 1186 | FRYLLK | 1 | Sequence | 0.888908 | 0.49 | <= |
| | 1101 | | YLLKFK | | | FKT | | | | | SB |
| | | | TEF | | | | | | | | |
| 440 | DRB1_ | 749 | FEVPFR | 4 | 1186 | FRYLLK | 1 | Sequence | 0.599939 | 2.37 | <= |
| | 1104 | | YLLKFK | | | FKT | | | | | WB |
| | | | TEF | | | | | | | | |
| 440 | DRB1_ | 749 | FEVPFR | 4 | 1186 | FRYLLK | 0.98 | Sequence | 0.310417 | 4.86 | <= |
| | 1401 | | YLLKFK | | | FKT | | | | | WB |
| | | | TEF | | | | | | | | |
| 440 | DRB1_ | 749 | FEVPFR | 4 | 1186 | FRYLLK | 0.98 | Sequence | 0.310417 | 4.86 | <= |
| | 1454 | | YLLKFK | | | FKT | | | | | WB |
| | | | TEF | | | | | | | | |
| 440 | DRB1_ | 749 | FEVPFR | 4 | 1186 | FRYLLK | 0.993 | Sequence | 0.239851 | 1.99 | <= |
| | 1402 | | YLLKFK | | | FKT | | | | | WB |
| | | | TEF | | | | | | | | |
| 440 | DRB1_ | 749 | FEVPFR | 4 | 1186 | FRYLLK | 0.973 | Sequence | 0.401092 | 3.49 | <= |
| | 1303 | | YLLKFK | | | FKT | | | | | WB |
| | | | TEF | | | | | | | | |
| 440 | DRB1_ | 749 | FEVPFR | 4 | 1186 | FRYLLK | 0.98 | Sequence | 0.470055 | 1.37 | <= |
| | 0803 | | YLLKFK | | | FKT | | | | | WB |
| | | | TEF | | | | | | | | |
| 441 | DRB1_ | 750 | EVPFRY | 3 | 1186 | FRYLLK | 0.96 | Sequence | 0.572362 | 2.55 | <= |
| | 0801 | | LLKFKT | | | FKT | | | | | WB |
| | | | EFL | | | | | | | | |
| 441 | DRB1_ | 750 | EVPFRY | 3 | 1186 | FRYLLK | 1 | Sequence | 0.829644 | 0.79 | <= |
| | 1101 | | LLKFKT | | | FKT | | | | | SB |
| | | | EFL | | | | | | | | |
| 441 | DRB1_ | 750 | EVPFRY | 3 | 1186 | FRYLLK | 1 | Sequence | 0.509261 | 3.33 | <= |
| | 1104 | | LLKFKT | | | FKT | | | | | WB |
| | | | EFL | | | | | | | | |
| 441 | DRB1_ | 750 | EVPFRY | 3 | 1186 | FRYLLK | 0.987 | Sequence | 0.202117 | 3.01 | <= |
| | 1402 | | LLKFKT | | | FKT | | | | | WB |
| | | | EFL | | | | | | | | |
| 441 | DRB1_ | 750 | EVPFRY | 3 | 1186 | FRYLLK | 0.933 | Sequence | 0.38845 | 2.43 | <= |
| | 0803 | | LLKFKT | | | FKT | | | | | WB |
| | | | EFL | | | | | | | | |
| 442 | DRB1_ | 751 | VPFRYL | 2 | 1186 | FRYLLK | 0.98 | Sequence | 0.483948 | 3.25 | <= |
| | 1101 | | LKFKTE | | | FKT | | | | | WB |
| | | | FLL | | | | | | | | |
| 443 | DRB1_ | 752 | PFRYLL | 4 | 1187 | LLKFKT | 0.96 | Sequence | 0.28912 | 3.44 | <= |
| | 1501 | | KFKTEF | | | EFL | | | | | WB |
| | | | LLS | | | | | | | | |
| 443 | DRB1_ | 752 | PFRYLL | 4 | 1187 | LLKFKT | 0.867 | Sequence | 0.215263 | 3.76 | <= |
| | 1503 | | KFKTEF | | | EFL | | | | | WB |
| | | | LLS | | | | | | | | |
| 444 | DRB1_ | 753 | FRYLLK | 3 | 1187 | LLKFKT | 0.94 | Sequence | 0.4561 | 1.94 | <= |
| | 1501 | | FKTEFL | | | EFL | | | | | WB |
| | | | LSD | | | | | | | | |
| 444 | DRB1_ | 753 | FRYLLK | 3 | 1187 | LLKFKT | 0.853 | Sequence | 0.351913 | 1.88 | <= |
| | 1503 | | FKTEFL | | | EFL | | | | | WB |
| | | | LSD | | | | | | | | |
| 445 | DRB1_ | 754 | RYLLKF | 2 | 1187 | LLKFKT | 0.88 | Sequence | 0.296402 | 3.35 | <= |
| | 1501 | | KTEFLL | | | EFL | | | | | WB |
| | | | SDA | | | | | | | | |
| 445 | DRB1_ | 754 | RYLLKF | 2 | 1187 | LLKFKT | 0.707 | Sequence | 0.286379 | 2.59 | <= |
| | 1503 | | KTEFLL | | | EFL | | | | | WB |
| | | | SDA | | | | | | | | |
| 449 | DRB1_ | 755 | KFKTEF | 5 | 1188 | FLLSDA | 1 | Sequence | 0.315798 | 3.64 | <= |
| | 0701 | | LLSDAL | | | LFL | | | | | WB |
| | | | FLV | | | | | | | | |
| 450 | DRB1_ | 756 | FKTEFL | 4 | 1188 | FLLSDA | 1 | Sequence | 0.303177 | 3.83 | <= |
| | 0701 | | LSDALF | | | LFL | | | | | WB |
| | | | LVV | | | | | | | | |
| 456 | DRB1_ | 757 | LSDALF | 4 | 1189 | LFLVVL | 0.873 | Sequence | 0.319806 | 3.74 | <= |
| | 1201 | | LVVLEH | | | EHY | | | | | WB |
| | | | YSD | | | | | | | | |
| 457 | DRB1_ | 758 | SDALFL | 3 | 1189 | LFLVVL | 0.74 | Sequence | 0.49333 | 1.42 | <= |
| | 1201 | | VVLEHY | | | EHY | | | | | WB |
| | | | SDP | | | | | | | | |
| 457 | DRB1_ | 758 | SDALFL | 4 | 1190 | FLVVLE | 0.473 | Sequence | 0.29476 | 4.64 | <= |
| | 0803 | | VVLEHY | | | HYS | | | | | WB |
| | | | SDP | | | | | | | | |
| 458 | DRB1_ | 759 | DALFLV | 4 | 1191 | LVVLEH | 0.627 | Sequence | 0.444188 | 4.56 | <= |
| | 0801 | | VLEHYS | | | YSD | | | | | WB |
| | | | DPY | | | | | | | | |
| 458 | DRB1_ | 759 | DALFLV | 2 | 1189 | LFLVVL | 0.48 | Sequence | 0.388842 | 2.52 | <= |
| | 1201 | | VLEHYS | | | EHY | | | | | WB |
| | | | DPY | | | | | | | | |
| 458 | DRB1_ | 759 | DALFLV | 4 | 1191 | LVVLEH | 0.593 | Sequence | 0.319064 | 3.92 | <= |
| | 0803 | | VLEHYS | | | YSD | | | | | WB |
| | | | DPY | | | | | | | | |
| 459 | DRB1_ | 760 | ALFLVV | 3 | 1191 | LVVLEH | 0.667 | Sequence | 0.280361 | 4.72 | <= |
| | 1201 | | LEHYSD | | | YSD | | | | | WB |
| | | | PYV | | | | | | | | |
| 460 | DRB1_ | 761 | LFLVVL | 5 | 1192 | LEHYSD | 1 | Sequence | 0.720759 | 0.75 | <= |
| | 1501 | | EHYSDP | | | PYV | | | | | SB |
| | | | YVN | | | | | | | | |
| 460 | DRB1_ | 761 | LFLVVL | 5 | 1192 | LEHYSD | 0.993 | Sequence | 0.563307 | 0.7 | <= |
| | 1503 | | EHYSDP | | | PYV | | | | | SB |
| | | | YVN | | | | | | | | |
| 461 | DRB1_ | 762 | FLVVLE | 4 | 1192 | LEHYSD | 1 | Sequence | 0.809381 | 0.49 | <= |
| | 1501 | | HYSDPY | | | PYV | | | | | SB |
| | | | VNI | | | | | | | | |
| 461 | DRB1_ | 762 | FLVVLE | 4 | 1192 | LEHYSD | 1 | Sequence | 0.657689 | 0.43 | <= |
| | 1503 | | HYSDPY | | | PYV | | | | | SB |
| | | | VNI | | | | | | | | |
| 462 | DRB1_ | 763 | LVVLEH | 3 | 1192 | LEHYSD | 1 | Sequence | 0.882104 | 0.3 | <= |
| | 1501 | | YSDPYV | | | PYV | | | | | SB |
| | | | NID | | | | | | | | |
| 462 | DRB1_ | 763 | LVVLEH | 3 | 1192 | LEHYSD | 1 | Sequence | 0.757543 | 0.23 | <= |
| | 1503 | | YSDPYV | | | PYV | | | | | SB |
| | | | NID | | | | | | | | |
| 463 | DRB1_ | 764 | VVLEHY | 2 | 1192 | LEHYSD | 1 | Sequence | 0.78488 | 0.56 | <= |
| | 1501 | | SDPYVN | | | PYV | | | | | SB |
| | | | IDF | | | | | | | | |
| 463 | DRB1_ | 764 | VVLEHY | 2 | 1192 | LEHYSD | 1 | Sequence | 0.635851 | 0.48 | <= |
| | 1503 | | SDPYVN | | | PYV | | | | | SB |
| | | | IDF | | | | | | | | |
| 464 | DRB1_ | 765 | VLEHYS | 1 | 1192 | LEHYSD | 0.98 | Sequence | 0.323703 | 3.03 | <= |
| | 1501 | | DPYVNI | | | PYV | | | | | WB |
| | | | DFR | | | | | | | | |
| 464 | DRB1_ | 765 | VLEHYS | 1 | 1192 | LEHYSD | 0.973 | Sequence | 0.25093 | 3.1 | <= |
| | 1503 | | DPYVNI | | | PYV | | | | | WB |
| | | | DFR | | | | | | | | |
| 467 | DRB1_ | 766 | HYSDPY | 6 | 1193 | VNIDFR | 1 | Sequence | 0.508586 | 1.98 | <= |
| | 0301 | | VNIDFR | | | DRI | | | | | WB |
| | | | DRI | | | | | | | | |
| 467 | DRB1_ | 766 | HYSDPY | 6 | 1193 | VNIDFR | 0.913 | Sequence | 0.15781 | 3.14 | <= |
| | 0305 | | VNIDFR | | | DRI | | | | | WB |
| | | | DRI | | | | | | | | |
| 468 | DRB1_ | 767 | YSDPYV | 5 | 1193 | VNIDFR | 1 | Sequence | 0.828965 | 0.47 | <= |
| | 0301 | | NIDFRD | | | DRI | | | | | SB |
| | | | RIE | | | | | | | | |
| 468 | DRB1_ | 767 | YSDPYV | 5 | 1193 | VNIDFR | 0.973 | Sequence | 0.395685 | 0.91 | <= |
| | 0305 | | NIDFRD | | | DRI | | | | | SB |
| | | | RIE | | | | | | | | |
| 468 | DRB1_ | 767 | YSDPYV | 5 | 1193 | VNIDFR | 0.967 | Sequence | 0.382481 | 3.26 | <= |
| | 1401 | | NIDFRD | | | DRI | | | | | WB |
| | | | RIE | | | | | | | | |
| 468 | DRB1_ | 767 | YSDPYV | 5 | 1193 | VNIDFR | 0.967 | Sequence | 0.382481 | 3.26 | <= |
| | 1454 | | NIDFRD | | | DRI | | | | | WB |
| | | | RIE | | | | | | | | |
| 469 | DRB1_ | 768 | SDPYVN | 4 | 1193 | VNIDFR | 1 | Sequence | 0.888846 | 0.29 | <= |
| | 0301 | | IDFRDR | | | DRI | | | | | SB |
| | | | IES | | | | | | | | |
| 469 | DRB1_ | 768 | SDPYVN | 4 | 1193 | VNIDFR | 0.987 | Sequence | 0.506695 | 0.5 | <= |
| | 0305 | | IDFRDR | | | DRI | | | | | SB |
| | | | IES | | | | | | | | |
| 469 | DRB1_ | 768 | SDPYVN | 4 | 1193 | VNIDFR | 0.98 | Sequence | 0.466279 | 2.07 | <= |
| | 1401 | | IDFRDR | | | DRI | | | | | WB |
| | | | IES | | | | | | | | |
| 469 | DRB1_ | 768 | SDPYVN | 4 | 1193 | VNIDFR | 0.98 | Sequence | 0.466279 | 2.07 | <= |
| | 1454 | | IDFRDR | | | DRI | | | | | WB |
| | | | IES | | | | | | | | |
| 470 | DRB1_ | 769 | DPYVNI | 3 | 1193 | VNIDFR | 1 | Sequence | 0.906572 | 0.23 | <= |
| | 0301 | | DFRDRI | | | DRI | | | | | SB |
| | | | ESA | | | | | | | | |
| 470 | DRB1_ | 769 | DPYVNI | 3 | 1193 | VNIDFR | 1 | Sequence | 0.556058 | 0.37 | <= |
| | 0305 | | DFRDRI | | | DRI | | | | | SB |
| | | | ESA | | | | | | | | |
| 470 | DRB1_ | 769 | DPYVNI | 5 | 1194 | IDFRDR | 0.687 | Sequence | 0.571573 | 2.66 | <= |
| | 1301 | | DFRDRI | | | IES | | | | | WB |
| | | | ESA | | | | | | | | |
| 470 | DRB1_ | 769 | DPYVNI | 3 | 1193 | VNIDFR | 0.98 | Sequence | 0.508246 | 1.66 | <= |
| | 1401 | | DFRDRI | | | DRI | | | | | WB |
| | | | ESA | | | | | | | | |
| 470 | DRB1_ | 769 | DPYVNI | 3 | 1193 | VNIDFR | 0.98 | Sequence | 0.508246 | 1.66 | <= |
| | 1454 | | DFRDRI | | | DRI | | | | | WB |
| | | | ESA | | | | | | | | |
| 470 | DRB1_ | 769 | DPYVNI | 3 | 1193 | VNIDFR | 0.88 | Sequence | 0.160084 | 4.74 | <= |
| | 1402 | | DFRDRI | | | DRI | | | | | WB |
| | | | ESA | | | | | | | | |
| 470 | DRB1_ | 769 | DPYVNI | 3 | 1193 | VNIDFR | 0.933 | Sequence | 0.325108 | 4.61 | <= |
| | 1303 | | DFRDRI | | | DRI | | | | | WB |
| | | | ESA | | | | | | | | |
| 471 | DRB1_ | 770 | PYVNID | 2 | 1193 | VNIDFR | 1 | Sequence | 0.800189 | 0.58 | <= |
| | 0301 | | FRDRIE | | | DRI | | | | | SB |
| | | | SAT | | | | | | | | |
| 471 | DRB1_ | 770 | PYVNID | 2 | 1193 | VNIDFR | 0.987 | Sequence | 0.33561 | 1.22 | <= |
| | 0305 | | FRDRIE | | | DRI | | | | | WB |
| | | | SAT | | | | | | | | |
| 471 | DRB1_ | 770 | PYVNID | 4 | 1194 | IDFRDR | 0.953 | Sequence | 0.3996 | 4.84 | <= |
| | 1104 | | FRDRIE | | | IES | | | | | WB |
| | | | SAT | | | | | | | | |
| 471 | DRB1_ | 770 | PYVNID | 4 | 1194 | IDFRDR | 0.947 | Sequence | 0.632878 | 1.98 | <= |
| | 1301 | | FRDRIE | | | IES | | | | | WB |
| | | | SAT | | | | | | | | |
| 472 | DRB1_ | 771 | YVNIDF | 1 | 1193 | VNIDFR | 0.973 | Sequence | 0.408831 | 2.73 | <= |
| | 0301 | | RDRIES | | | DRI | | | | | WB |
| | | | ATF | | | | | | | | |
| 472 | DRB1_ | 771 | YVNIDF | 3 | 1194 | IDFRDR | 0.98 | Sequence | 0.610475 | 2.22 | <= |
| | 1301 | | RDRIES | | | IES | | | | | WB |
| | | | ATF | | | | | | | | |
| 473 | DRB1_ | 772 | VNIDFR | 2 | 1194 | IDFRDR | 0.993 | Sequence | 0.44424 | 4.66 | <= |
| | 1301 | | DRIESA | | | IES | | | | | WB |
| | | | TFF | | | | | | | | |
| 483 | DRB1_ | 773 | SATFFS | 4 | 1195 | FSLLNL | 0.827 | Sequence | 0.27446 | 4.42 | <= |
| | 0405 | | LLNLDK | | | DKD | | | | | WB |
| | | | DSV | | | | | | | | |
| 484 | DRB1_ | 774 | ATFFSL | 3 | 1195 | FSLLNL | 0.947 | Sequence | 0.444241 | 3.73 | <= |
| | 0403 | | LNLDKD | | | DKD | | | | | WB |
| | | | SVK | | | | | | | | |
| 484 | DRB1_ | 774 | ATFFSL | 3 | 1195 | FSLLNL | 0.953 | Sequence | 0.366016 | 3.05 | <= |
| | 0405 | | LNLDKD | | | DKD | | | | | WB |
| | | | SVK | | | | | | | | |
| 485 | DRB1_ | 775 | TFFSLL | 5 | 1196 | LNLDKD | 0.887 | Sequence | 0.245324 | 4.6 | <= |
| | 0301 | | NLDKDS | | | SVK | | | | | WB |
| | | | VKE | | | | | | | | |
| 486 | DRB1_ | 776 | FFSLLN | 4 | 1196 | LNLDKD | 0.927 | Sequence | 0.401912 | 2.79 | <= |
| | 0301 | | LDKDSV | | | SVK | | | | | WB |
| | | | KEQ | | | | | | | | |
| 487 | DRB1_ | 777 | FSLLNL | 3 | 1196 | LNLDKD | 0.94 | Sequence | 0.445614 | 2.43 | <= |
| | 0301 | | DKDSVK | | | SVK | | | | | WB |
| | | | EQT | | | | | | | | |
| 487 | DRB1_ | 777 | FSLLNL | 3 | 1196 | LNLDKD | 0.767 | Sequence | 0.097033 | 4.96 | <= |
| | 0305 | | DKDSVK | | | SVK | | | | | WB |
| | | | EQT | | | | | | | | |
| 488 | DRB1_ | 778 | SLLNLD | 2 | 1196 | LNLDKD | 0.733 | Sequence | 0.333446 | 3.4 | <= |
| | 0301 | | KDSVKE | | | SVK | | | | | WB |
| | | | QTS | | | | | | | | |
| 493 | DRB1_ | 779 | DKDSVK | 4 | 1197 | VKEQTS | 1 | Sequence | 0.366894 | 4.65 | <= |
| | 0102 | | EQTSIS | | | IST | | | | | WB |
| | | | TFD | | | | | | | | |
| 494 | DRB1_ | 780 | KDSVKE | 3 | 1197 | VKEQTS | 1 | Sequence | 0.363887 | 4.7 | <= |
| | 0102 | | QTSIST | | | IST | | | | | WB |
| | | | FDW | | | | | | | | |
| 506 | DRB1_ | 781 | FDWNLS | 4 | 1198 | LSSFYK | 0.993 | Sequence | 0.589455 | 2.45 | <= |
| | 1301 | | SFYKRT | | | RTF | | | | | WB |
| | | | FND | | | | | | | | |
| 507 | DRB1_ | 782 | DWNLSS | 3 | 1198 | LSSFYK | 0.993 | Sequence | 0.689689 | 1.4 | <= |
| | 1301 | | FYKRTF | | | RTF | | | | | WB |
| | | | NDG | | | | | | | | |
| 510 | DRB1_ | 783 | LSSFYK | 4 | 1199 | YKRTFN | 1 | Sequence | 0.298989 | 4.55 | <= |
| | 0401 | | RTFNDG | | | DGS | | | | | WB |
| | | | SIL | | | | | | | | |
| 511 | DRB1_ | 784 | SSFYKR | 3 | 1199 | YKRTFN | 1 | Sequence | 0.43473 | 2.68 | <= |
| | 0401 | | TFNDGS | | | DGS | | | | | WB |
| | | | ILD | | | | | | | | |
| 511 | DRB1_ | 784 | SSFYKR | 3 | 1199 | YKRTFN | 1 | Sequence | 0.411363 | 3.53 | <= |
| | 0408 | | TFNDGS | | | DGS | | | | | WB |
| | | | ILD | | | | | | | | |
| 518 | DRB1_ | 785 | FNDGSI | 5 | 1200 | ILDYKL | 1 | Sequence | 0.380986 | 2.5 | <= |
| | 1501 | | LDYKLN | | | NNL | | | | | WB |
| | | | NLG | | | | | | | | |
| 518 | DRB1_ | 785 | FNDGSI | 5 | 1200 | ILDYKL | 0.993 | Sequence | 0.312498 | 2.28 | <= |
| | 1503 | | LDYKLN | | | NNL | | | | | WB |
| | | | NLG | | | | | | | | |
| 519 | DRB1_ | 786 | NDGSIL | 5 | 1201 | LDYKLN | 0.84 | Sequence | 0.363379 | 2.93 | <= |
| | 0402 | | DYKLNN | | | NLG | | | | | WB |
| | | | LGL | | | | | | | | |
| 519 | DRB1_ | 786 | NDGSIL | 4 | 1200 | ILDYKL | 0.993 | Sequence | 0.406854 | 2.3 | <= |
| | 1501 | | DYKLNN | | | NNL | | | | | WB |
| | | | LGL | | | | | | | | |
| 519 | DRB1_ | 786 | NDGSIL | 4 | 1200 | ILDYKL | 0.96 | Sequence | 0.32039 | 2.19 | <= |
| | 1503 | | DYKLNN | | | NNL | | | | | WB |
| | | | LGL | | | | | | | | |
| 520 | DRB1_ | 787 | DGSILD | 4 | 1201 | LDYKLN | 0.893 | Sequence | 0.574794 | 1.1 | <= |
| | 0402 | | YKLNNL | | | NLG | | | | | WB |
| | | | GLN | | | | | | | | |
| 520 | DRB1_ | 787 | DGSILD | 3 | 1200 | ILDYKL | 0.98 | Sequence | 0.492043 | 1.73 | <= |
| | 1501 | | YKLNNL | | | NNL | | | | | WB |
| | | | GLN | | | | | | | | |
| 520 | DRB1_ | 787 | DGSILD | 3 | 1200 | ILDYKL | 0.94 | Sequence | 0.40074 | 1.51 | <= |
| | 1503 | | YKLNNL | | | NNL | | | | | WB |
| | | | GLN | | | | | | | | |
| 521 | DRB1_ | 788 | GSILDY | 3 | 1201 | LDYKLN | 0.973 | Sequence | 0.54784 | 1.25 | <= |
| | 0402 | | KLNNLG | | | NLG | | | | | WB |
| | | | LNF | | | | | | | | |
| 521 | DRB1_ | 788 | GSILDY | 2 | 1200 | ILDYKL | 0.84 | Sequence | 0.249417 | 3.98 | <= |
| | 1501 | | KLNNLG | | | NNL | | | | | WB |
| | | | LNF | | | | | | | | |
| 521 | DRB1_ | 788 | GSILDY | 2 | 1200 | ILDYKL | 0.827 | Sequence | 0.196977 | 4.19 | <= |
| | 1503 | | KLNNLG | | | NNL | | | | | WB |
| | | | LNF | | | | | | | | |
| 531 | DRB1_ | 789 | LGLNFK | 6 | 1202 | LSGYEN | 0.993 | Sequence | 0.270462 | 3.68 | <= |
| | 1501 | | LSGYEN | | | LYV | | | | | WB |
| | | | LYV | | | | | | | | |
| 531 | DRB1_ | 789 | LGLNFK | 6 | 1202 | LSGYEN | 0.973 | Sequence | 0.181709 | 4.61 | <= |
| | 1503 | | LSGYEN | | | LYV | | | | | WB |
| | | | LYV | | | | | | | | |
| 532 | DRB1_ | 790 | GLNFKL | 5 | 1202 | LSGYEN | 1 | Sequence | 0.848063 | 0.39 | <= |
| | 1501 | | SGYENL | | | LYV | | | | | SB |
| | | | YVK | | | | | | | | |
| 532 | DRB1_ | 790 | GLNFKL | 5 | 1202 | LSGYEN | 1 | Sequence | 0.662046 | 0.42 | <= |
| | 1503 | | SGYENL | | | LYV | | | | | SB |
| | | | YVK | | | | | | | | |
| 533 | DRB1_ | 791 | LNFKLS | 4 | 1202 | LSGYEN | 1 | Sequence | 0.374378 | 2.79 | <= |
| | 0402 | | GYENLY | | | LYV | | | | | WB |
| | | | VKS | | | | | | | | |
| 533 | DRB1_ | 791 | LNFKLS | 4 | 1202 | LSGYEN | 1 | Sequence | 0.93304 | 0.16 | <= |
| | 1501 | | GYENLY | | | LYV | | | | | SB |
| | | | VKS | | | | | | | | |
| 533 | DRB1_ | 791 | LNFKLS | 4 | 1202 | LSGYEN | 1 | Sequence | 0.441282 | 3.2 | <= |
| | 1601 | | GYENLY | | | LYV | | | | | WB |
| | | | VKS | | | | | | | | |
| 533 | DRB1_ | 791 | LNFKLS | 4 | 1202 | LSGYEN | 1 | Sequence | 0.79218 | 0.17 | <= |
| | 1503 | | GYENLY | | | LYV | | | | | SB |
| | | | VKS | | | | | | | | |
| 534 | DRB1_ | 792 | NFKLSG | 3 | 1202 | LSGYEN | 1 | Sequence | 0.483072 | 1.69 | <= |
| | 0402 | | YENLYV | | | LYV | | | | | WB |
| | | | KSP | | | | | | | | |
| 534 | DRB1_ | 792 | NFKLSG | 3 | 1202 | LSGYEN | 1 | Sequence | 0.962263 | 0.07 | <= |
| | 1501 | | YENLYV | | | LYV | | | | | SB |
| | | | KSP | | | | | | | | |
| 534 | DRB1_ | 792 | NFKLSG | 3 | 1202 | LSGYEN | 1 | Sequence | 0.561731 | 1.9 | <= |
| | 1601 | | YENLYV | | | LYV | | | | | WB |
| | | | KSP | | | | | | | | |
| 534 | DRB1_ | 792 | NFKLSG | 3 | 1202 | LSGYEN | 1 | Sequence | 0.863485 | 0.07 | <= |
| | 1503 | | YENLYV | | | LYV | | | | | SB |
| | | | KSP | | | | | | | | |
| 535 | DRB1_ | 793 | FKLSGY | 2 | 1202 | LSGYEN | 1 | Sequence | 0.857641 | 0.37 | <= |
| | 1501 | | ENLYVK | | | LYV | | | | | SB |
| | | | SPL | | | | | | | | |
| 535 | DRB1_ | 793 | FKLSGY | 2 | 1202 | LSGYEN | 1 | Sequence | 0.677092 | 0.38 | <= |
| | 1503 | | ENLYVK | | | LYV | | | | | SB |
| | | | SPL | | | | | | | | |
| 536 | DRB1_ | 794 | KLSGYE | 1 | 1202 | LSGYEN | 1 | Sequence | 0.550847 | 1.43 | <= |
| | 1501 | | NLYVKS | | | LYV | | | | | WB |
| | | | PLE | | | | | | | | |
| 536 | DRB1_ | 794 | KLSGYE | 1 | 1202 | LSGYEN | 0.987 | Sequence | 0.400437 | 1.51 | <= |
| | 1503 | | NLYVKS | | | LYV | | | | | WB |
| | | | PLE | | | | | | | | |
| 540 | DRB1_ | 795 | YENLYV | 3 | 1203 | LYVKSP | 0.993 | Sequence | 0.467295 | 4.23 | <= |
| | 1301 | | KSPLEK | | | LEK | | | | | WB |
| | | | PKE | | | | | | | | |
| 556 | DRB1_ | 796 | NDPTRK | 6 | 1204 | WFYLER | 1 | Sequence | 0.406029 | 4.15 | <= |
| | 1101 | | WFYLER | | | IYA | | | | | WB |
| | | | IYA | | | | | | | | |
| 557 | DRB1_ | 797 | DPTRKW | 5 | 1204 | WFYLER | 0.98 | Sequence | 0.607338 | 2.16 | <= |
| | 0801 | | FYLERI | | | IYA | | | | | WB |
| | | | YAP | | | | | | | | |
| 557 | DRB1_ | 797 | DPTRKW | 5 | 1204 | WFYLER | 1 | Sequence | 0.869997 | 0.59 | <= |
| | 1101 | | FYLERI | | | IYA | | | | | SB |
| | | | YAP | | | | | | | | |
| 557 | DRB1_ | 797 | DPTRKW | 5 | 1204 | WFYLER | 1 | Sequence | 0.432369 | 4.33 | <= |
| | 1104 | | FYLERI | | | IYA | | | | | WB |
| | | | YAP | | | | | | | | |
| 557 | DRB1_ | 797 | DPTRKW | 5 | 1204 | WFYLER | 0.98 | Sequence | 0.270827 | 1.47 | <= |
| | 1402 | | FYLERI | | | IYA | | | | | WB |
| | | | YAP | | | | | | | | |
| 557 | DRB1_ | 797 | DPTRKW | 5 | 1204 | WFYLER | 1 | Sequence | 0.406093 | 3.43 | <= |
| | 1303 | | FYLERI | | | IYA | | | | | WB |
| | | | YAP | | | | | | | | |
| 557 | DRB1_ | 797 | DPTRKW | 5 | 1204 | WFYLER | 0.967 | Sequence | 0.445412 | 1.62 | <= |
| | 0803 | | FYLERI | | | IYA | | | | | WB |
| | | | YAP | | | | | | | | |
| 558 | DRB1_ | 798 | PTRKWF | 4 | 1204 | WFYLER | 0.98 | Sequence | 0.634112 | 1.87 | <= |
| | 0801 | | YLERIY | | | IYA | | | | | WB |
| | | | APY | | | | | | | | |
| 558 | DRB1_ | 798 | PTRKWF | 4 | 1204 | WFYLER | 1 | Sequence | 0.893672 | 0.47 | <= |
| | 1101 | | YLERIY | | | IYA | | | | | SB |
| | | | APY | | | | | | | | |
| 558 | DRB1_ | 798 | PTRKWF | 4 | 1204 | WFYLER | 1 | Sequence | 0.499355 | 3.44 | <= |
| | 1104 | | YLERIY | | | IYA | | | | | WB |
| | | | APY | | | | | | | | |
| 558 | DRB1_ | 798 | PTRKWF | 4 | 1204 | WFYLER | 0.967 | Sequence | 0.335217 | 0.79 | <= |
| | 1402 | | YLERIY | | | IYA | | | | | SB |
| | | | APY | | | | | | | | |
| 558 | DRB1_ | 798 | PTRKWF | 4 | 1204 | WFYLER | 0.987 | Sequence | 0.441741 | 3.01 | <= |
| | 1303 | | YLERIY | | | IYA | | | | | WB |
| | | | APY | | | | | | | | |
| 558 | DRB1_ | 798 | PTRKWF | 4 | 1204 | WFYLER | 0.967 | Sequence | 0.488594 | 1.2 | <= |
| | 0803 | | YLERIY | | | IYA | | | | | WB |
| | | | APY | | | | | | | | |
| 559 | DRB1_ | 799 | TRKWFY | 3 | 1204 | WFYLER | 0.96 | Sequence | 0.55127 | 2.82 | <= |
| | 0801 | | LERIYA | | | IYA | | | | | WB |
| | | | PYI | | | | | | | | |
| 559 | DRB1_ | 799 | TRKWFY | 3 | 1204 | WFYLER | 1 | Sequence | 0.845422 | 0.71 | <= |
| | 1101 | | LERIYA | | | IYA | | | | | SB |
| | | | PYI | | | | | | | | |
| 559 | DRB1_ | 799 | TRKWFY | 3 | 1204 | WFYLER | 0.993 | Sequence | 0.43044 | 4.36 | <= |
| | 1104 | | LERIYA | | | IYA | | | | | WB |
| | | | PYI | | | | | | | | |
| 559 | DRB1_ | 799 | TRKWFY | 3 | 1204 | WFYLER | 0.96 | Sequence | 0.314238 | 0.97 | <= |
| | 1402 | | LERIYA | | | IYA | | | | | SB |
| | | | PYI | | | | | | | | |
| 559 | DRB1_ | 799 | TRKWFY | 3 | 1204 | WFYLER | 0.967 | Sequence | 0.359136 | 4.07 | <= |
| | 1303 | | LERIYA | | | IYA | | | | | WB |
| | | | PYI | | | | | | | | |
| 559 | DRB1_ | 799 | TRKWFY | 3 | 1204 | WFYLER | 0.933 | Sequence | 0.420273 | 1.95 | <= |
| | 0803 | | LERIYA | | | IYA | | | | | WB |
| | | | PYI | | | | | | | | |
| 560 | DRB1_ | 800 | RKWFYL | 2 | 1204 | WFYLER | 1 | Sequence | 0.636078 | 1.93 | <= |
| | 1101 | | ERIYAP | | | IYA | | | | | WB |
| | | | YID | | | | | | | | |
| 560 | DRB1_ | 800 | RKWFYL | 2 | 1204 | WFYLER | 0.8 | Sequence | 0.185644 | 3.61 | <= |
| | 1402 | | ERIYAP | | | IYA | | | | | WB |
| | | | YID | | | | | | | | |
| 564 | DRB1_ | 801 | YLERIY | 5 | 1205 | YAPYID | 0.92 | Sequence | 0.182889 | 4.58 | <= |
| | 1503 | | APYIDL | | | LNF | | | | | WB |
| | | | NFQ | | | | | | | | |
| 565 | DRB1_ | 802 | LERIYA | 4 | 1205 | YAPYID | 1 | Sequence | 0.380375 | 2.51 | <= |
| | 1501 | | PYIDLN | | | LNF | | | | | WB |
| | | | FQK | | | | | | | | |
| 565 | DRB1_ | 802 | LERIYA | 4 | 1205 | YAPYID | 1 | Sequence | 0.34933 | 1.9 | <= |
| | 1503 | | PYIDLN | | | LNF | | | | | WB |
| | | | FQK | | | | | | | | |
| 566 | DRB1_ | 803 | ERIYAP | 3 | 1205 | YAPYID | 1 | Sequence | 0.494402 | 1.72 | <= |
| | 1501 | | YIDLNF | | | LNF | | | | | WB |
| | | | QKD | | | | | | | | |
| 566 | DRB1_ | 803 | ERIYAP | 3 | 1205 | YAPYID | 1 | Sequence | 0.384595 | 3.93 | <= |
| | 1601 | | YIDLNF | | | LNF | | | | | WB |
| | | | QKD | | | | | | | | |
| 566 | DRB1_ | 803 | ERIYAP | 3 | 1205 | YAPYID | 1 | Sequence | 0.449946 | 1.2 | <= |
| | 1503 | | YIDLNF | | | LNF | | | | | WB |
| | | | QKD | | | | | | | | |
| 566 | DRB1_ | 803 | ERIYAP | 3 | 1205 | YAPYID | 0.733 | Sequence | 0.285081 | 4.96 | <= |
| | 0803 | | YIDLNF | | | LNF | | | | | WB |
| | | | QKD | | | | | | | | |
| 567 | DRB1_ | 804 | RIYAPY | 2 | 1205 | YAPYID | 0.993 | Sequence | 0.259097 | 3.84 | <= |
| | 1501 | | IDLNFQ | | | LNF | | | | | WB |
| | | | KDL | | | | | | | | |
| 567 | DRB1_ | 804 | RIYAPY | 2 | 1205 | YAPYID | 0.967 | Sequence | 0.245604 | 3.19 | <= |
| | 1503 | | IDLNFQ | | | LNF | | | | | WB |
| | | | KDL | | | | | | | | |
| 570 | DRB1_ | 805 | APYIDL | 5 | 1206 | LNFQKD | 0.967 | Sequence | 0.41453 | 4.25 | <= |
| | 0403 | | NFQKDL | | | LYN | | | | | WB |
| | | | YNN | | | | | | | | |
| 571 | DRB1_ | 806 | PYIDLN | 4 | 1206 | LNFQKD | 1 | Sequence | 0.465059 | 1.83 | <= |
| | 0402 | | FQKDLY | | | LYN | | | | | WB |
| | | | NNQ | | | | | | | | |
| 571 | DRB1_ | 806 | PYIDLN | 4 | 1206 | LNFQKD | 1 | Sequence | 0.56875 | 2.14 | <= |
| | 0403 | | FQKDLY | | | LYN | | | | | WB |
| | | | NNQ | | | | | | | | |
| 571 | DRB1_ | 806 | PYIDLN | 4 | 1206 | LNFQKD | 1 | Sequence | 0.525166 | 2.46 | <= |
| | 0404 | | FQKDLY | | | LYN | | | | | WB |
| | | | NNQ | | | | | | | | |
| 572 | DRB1_ | 807 | YIDLNF | 3 | 1206 | LNFQKD | 1 | Sequence | 0.458669 | 1.89 | <= |
| | 0402 | | QKDLYN | | | LYN | | | | | WB |
| | | | NQW | | | | | | | | |
| 572 | DRB1_ | 807 | YIDLNF | 3 | 1206 | LNFQKD | 1 | Sequence | 0.544125 | 2.4 | <= |
| | 0403 | | QKDLYN | | | LYN | | | | | WB |
| | | | NQW | | | | | | | | |
| 572 | DRB1_ | 807 | YIDLNF | 3 | 1206 | LNFQKD | 1 | Sequence | 0.504414 | 2.66 | <= |
| | 0404 | | QKDLYN | | | LYN | | | | | WB |
| | | | NQW | | | | | | | | |
| 573 | DRB1_ | 808 | IDLNFQ | 2 | 1206 | LNFQKD | 0.993 | Sequence | 0.257578 | 4.87 | <= |
| | 0402 | | KDLYNN | | | LYN | | | | | WB |
| | | | QWT | | | | | | | | |
| 581 | DRB1_ | 809 | LYNNQW | 5 | 1207 | WTFSTD | 0.993 | Sequence | 0.421497 | 4.12 | <= |
| | 0403 | | TFSTDT | | | TKE | | | | | WB |
| | | | KEM | | | | | | | | |
| 581 | DRB1_ | 809 | LYNNQW | 5 | 1207 | WTFSTD | 1 | Sequence | 0.54494 | 1.56 | <= |
| | 0405 | | TFSTDT | | | TKE | | | | | WB |
| | | | KEM | | | | | | | | |
| 582 | DRB1_ | 810 | YNNQWT | 6 | 1208 | FSTDTK | 0.98 | Sequence | 0.281851 | 1.6 | <= |
| | 0305 | | FSTDTK | | | EMI | | | | | WB |
| | | | EMI | | | | | | | | |
| 582 | DRB1_ | 810 | YNNQWT | 4 | 1207 | WTFSTD | 0.993 | Sequence | 0.500119 | 2.95 | <= |
| | 0403 | | FSTDTK | | | TKE | | | | | WB |
| | | | EMI | | | | | | | | |
| 582 | DRB1_ | 810 | YNNQWT | 4 | 1207 | WTFSTD | 1 | Sequence | 0.629299 | 1.14 | <= |
| | 0405 | | FSTDTK | | | TKE | | | | | WB |
| | | | EMI | | | | | | | | |
| 583 | DRB1_ | 811 | NNQWTF | 5 | 1208 | FSTDTK | 1 | Sequence | 0.377942 | 2.99 | <= |
| | 0301 | | STDTKE | | | EMI | | | | | WB |
| | | | MIM | | | | | | | | |
| 583 | DRB1_ | 811 | NNQWTF | 5 | 1208 | FSTDTK | 0.993 | Sequence | 0.536404 | 0.41 | <= |
| | 0305 | | STDTKE | | | EMI | | | | | SB |
| | | | MIM | | | | | | | | |
| 583 | DRB1_ | 811 | NNQWTF | 3 | 1207 | WTFSTD | 1 | Sequence | 0.472573 | 3.31 | <= |
| | 0403 | | STDTKE | | | TKE | | | | | WB |
| | | | MIM | | | | | | | | |
| 583 | DRB1_ | 811 | NNQWTF | 3 | 1207 | WTFSTD | 1 | Sequence | 0.58979 | 1.33 | <= |
| | 0405 | | STDTKE | | | TKE | | | | | WB |
| | | | MIM | | | | | | | | |
| 583 | DRB1_ | 811 | NNQWTF | 5 | 1208 | FSTDTK | 0.707 | Sequence | 0.393608 | 3.59 | <= |
| | 1303 | | STDTKE | | | EMI | | | | | WB |
| | | | MIM | | | | | | | | |
| 584 | DRB1_ | 812 | NQWTFS | 4 | 1208 | FSTDTK | 1 | Sequence | 0.562723 | 1.67 | <= |
| | 0301 | | TDTKEM | | | EMI | | | | | WB |
| | | | IMR | | | | | | | | |
| 584 | DRB1_ | 812 | NQWTFS | 4 | 1208 | FSTDTK | 1 | Sequence | 0.698445 | 0.12 | <= |
| | 0305 | | TDTKEM | | | EMI | | | | | SB |
| | | | IMR | | | | | | | | |
| 584 | DRB1_ | 812 | NQWTFS | 4 | 1208 | FSTDTK | 0.94 | Sequence | 0.189755 | 3.45 | <= |
| | 1402 | | TDTKEM | | | EMI | | | | | WB |
| | | | IMR | | | | | | | | |
| 584 | DRB1_ | 812 | NQWTFS | 4 | 1208 | FSTDTK | 0.993 | Sequence | 0.525725 | 2.15 | <= |
| | 1303 | | TDTKEM | | | EMI | | | | | WB |
| | | | IMR | | | | | | | | |
| 585 | DRB1_ | 813 | QWTFST | 3 | 1208 | FSTDTK | 1 | Sequence | 0.649739 | 1.22 | <= |
| | 0301 | | DTKEMI | | | EMI | | | | | WB |
| | | | MRP | | | | | | | | |
| 585 | DRB1_ | 813 | QWTFST | 3 | 1208 | FSTDTK | 1 | Sequence | 0.767305 | 0.06 | <= |
| | 0305 | | DTKEMI | | | EMI | | | | | SB |
| | | | MRP | | | | | | | | |
| 585 | DRB1_ | 813 | QWTFST | 3 | 1208 | FSTDTK | 1 | Sequence | 0.256317 | 1.7 | <= |
| | 1402 | | DTKEMI | | | EMI | | | | | WB |
| | | | MRP | | | | | | | | |
| 585 | DRB1_ | 813 | QWTFST | 3 | 1208 | FSTDTK | 1 | Sequence | 0.620321 | 1.41 | <= |
| | 1303 | | DTKEMI | | | EMI | | | | | WB |
| | | | MRP | | | | | | | | |
| 586 | DRB1_ | 814 | WTFSTD | 2 | 1208 | FSTDTK | 1 | Sequence | 0.411619 | 2.71 | <= |
| | 0301 | | TKEMIM | | | EMI | | | | | WB |
| | | | RPE | | | | | | | | |
| 586 | DRB1_ | 814 | WTFSTD | 2 | 1208 | FSTDTK | 1 | Sequence | 0.554968 | 0.37 | <= |
| | 0305 | | TKEMIM | | | EMI | | | | | SB |
| | | | RPE | | | | | | | | |
| 586 | DRB1_ | 814 | WTFSTD | 2 | 1208 | FSTDTK | 1 | Sequence | 0.362597 | 4.02 | <= |
| | 1303 | | TKEMIM | | | EMI | | | | | WB |
| | | | RPE | | | | | | | | |
| 587 | DRB1_ | 815 | TFSTDT | 1 | 1208 | FSTDTK | 1 | Sequence | 0.272696 | 1.68 | <= |
| | 0305 | | KEMIMR | | | EMI | | | | | WB |
| | | | PEI | | | | | | | | |
| 591 | DRB1_ | 816 | DTKEMI | 6 | 1209 | MRPEIK | 0.98 | Sequence | 0.346704 | 4.26 | <= |
| | 1303 | | MRPEIK | | | NLE | | | | | WB |
| | | | NLE | | | | | | | | |
| 592 | DRB1_ | 817 | TKEMIM | 5 | 1209 | MRPEIK | 0.987 | Sequence | 0.231455 | 4.84 | <= |
| | 0301 | | RPEIKN | | | NLE | | | | | WB |
| | | | LED | | | | | | | | |
| 592 | DRB1_ | 817 | TKEMIM | 5 | 1209 | MRPEIK | 0.727 | Sequence | 0.573128 | 2.54 | <= |
| | 0801 | | RPEIKN | | | NLE | | | | | WB |
| | | | LED | | | | | | | | |
| 592 | DRB1_ | 817 | TKEMIM | 5 | 1209 | MRPEIK | 1 | Sequence | 0.812816 | 0.47 | <= |
| | 1303 | | RPEIKN | | | NLE | | | | | SB |
| | | | LED | | | | | | | | |
| 592 | DRB1_ | 817 | TKEMIM | 5 | 1209 | MRPEIK | 0.713 | Sequence | 0.284733 | 4.98 | <= |
| | 0803 | | RPEIKN | | | NLE | | | | | WB |
| | | | LED | | | | | | | | |
| 593 | DRB1_ | 818 | KEMIMR | 4 | 1209 | MRPEIK | 0.993 | Sequence | 0.402615 | 2.78 | <= |
| | 0301 | | PEIKNL | | | NLE | | | | | WB |
| | | | EDK | | | | | | | | |
| 593 | DRB1_ | 818 | KEMIMR | 4 | 1209 | MRPEIK | 0.973 | Sequence | 0.110309 | 4.42 | <= |
| | 0305 | | PEIKNL | | | NLE | | | | | WB |
| | | | EDK | | | | | | | | |
| 593 | DRB1_ | 818 | KEMIMR | 4 | 1209 | MRPEIK | 0.987 | Sequence | 0.726992 | 1.1 | <= |
| | 0801 | | PEIKNL | | | NLE | | | | | WB |
| | | | EDK | | | | | | | | |
| 593 | DRB1_ | 818 | KEMIMR | 4 | 1209 | MRPEIK | 0.96 | Sequence | 0.466236 | 3.87 | <= |
| | 1104 | | PEIKNL | | | NLE | | | | | WB |
| | | | EDK | | | | | | | | |
| 593 | DRB1_ | 818 | KEMIMR | 4 | 1209 | MRPEIK | 0.987 | Sequence | 0.402733 | 2.9 | <= |
| | 1401 | | PEIKNL | | | NLE | | | | | WB |
| | | | EDK | | | | | | | | |
| 593 | DRB1_ | 818 | KEMIMR | 4 | 1209 | MRPEIK | 0.987 | Sequence | 0.402733 | 2.9 | <= |
| | 1454 | | PEIKNL | | | NLE | | | | | WB |
| | | | EDK | | | | | | | | |
| 593 | DRB1_ | 818 | KEMIMR | 4 | 1209 | MRPEIK | 0.953 | Sequence | 0.166362 | 4.41 | <= |
| | 1402 | | PEIKNL | | | NLE | | | | | WB |
| | | | EDK | | | | | | | | |
| 593 | DRB1_ | 818 | KEMIMR | 4 | 1209 | MRPEIK | 1 | Sequence | 0.907109 | 0.15 | <= |
| | 1303 | | PEIKNL | | | NLE | | | | | SB |
| | | | EDK | | | | | | | | |
| 593 | DRB1_ | 818 | KEMIMR | 4 | 1209 | MRPEIK | 0.953 | Sequence | 0.402083 | 2.22 | <= |
| | 0803 | | PEIKNL | | | NLE | | | | | WB |
| | | | EDK | | | | | | | | |
| 594 | DRB1_ | 819 | EMIMRP | 3 | 1209 | MRPEIK | 1 | Sequence | 0.487697 | 2.13 | <= |
| | 0301 | | EIKNLE | | | NLE | | | | | WB |
| | | | DKD | | | | | | | | |
| 594 | DRB1_ | 819 | EMIMRP | 3 | 1209 | MRPEIK | 1 | Sequence | 0.154231 | 3.22 | <= |
| | 0305 | | EIKNLE | | | NLE | | | | | WB |
| | | | DKD | | | | | | | | |
| 594 | DRB1_ | 819 | EMIMRP | 3 | 1209 | MRPEIK | 1 | Sequence | 0.791498 | 0.66 | <= |
| | 0801 | | EIKNLE | | | NLE | | | | | SB |
| | | | DKD | | | | | | | | |
| 594 | DRB1_ | 819 | EMIMRP | 3 | 1209 | MRPEIK | 1 | Sequence | 0.418147 | 4.01 | <= |
| | 1101 | | EIKNLE | | | NLE | | | | | WB |
| | | | DKD | | | | | | | | |
| 594 | DRB1_ | 819 | EMIMRP | 3 | 1209 | MRPEIK | 1 | Sequence | 0.579768 | 2.56 | <= |
| | 1104 | | EIKNLE | | | NLE | | | | | WB |
| | | | DKD | | | | | | | | |
| 594 | DRB1_ | 819 | EMIMRP | 3 | 1209 | MRPEIK | 0.993 | Sequence | 0.477463 | 1.94 | <= |
| | 1401 | | EIKNLE | | | NLE | | | | | WB |
| | | | DKD | | | | | | | | |
| 594 | DRB1_ | 819 | EMIMRP | 3 | 1209 | MRPEIK | 0.993 | Sequence | 0.477463 | 1.94 | <= |
| | 1454 | | EIKNLE | | | NLE | | | | | WB |
| | | | DKD | | | | | | | | |
| 594 | DRB1_ | 819 | EMIMRP | 3 | 1209 | MRPEIK | 0.98 | Sequence | 0.217427 | 2.57 | <= |
| | 1402 | | EIKNLE | | | NLE | | | | | WB |
| | | | DKD | | | | | | | | |
| 594 | DRB1_ | 819 | EMIMRP | 3 | 1209 | MRPEIK | 1 | Sequence | 0.929964 | 0.09 | <= |
| | 1303 | | EIKNLE | | | NLE | | | | | SB |
| | | | DKD | | | | | | | | |
| 594 | DRB1_ | 819 | EMIMRP | 3 | 1209 | MRPEIK | 0.987 | Sequence | 0.324506 | 4.09 | <= |
| | 1302 | | EIKNLE | | | NLE | | | | | WB |
| | | | DKD | | | | | | | | |
| 594 | DRB1_ | 819 | EMIMRP | 3 | 1209 | MRPEIK | 0.993 | Sequence | 0.477738 | 1.3 | <= |
| | 0803 | | EIKNLE | | | NLE | | | | | WB |
| | | | DKD | | | | | | | | |
| 595 | DRB1_ | 820 | MIMRPE | 2 | 1209 | MRPEIK | 0.993 | Sequence | 0.264172 | 4.3 | <= |
| | 0301 | | IKNLED | | | NLE | | | | | WB |
| | | | KDN | | | | | | | | |
| 595 | DRB1_ | 820 | MIMRPE | 2 | 1209 | MRPEIK | 1 | Sequence | 0.549502 | 2.84 | <= |
| | 0801 | | IKNLED | | | NLE | | | | | WB |
| | | | KDN | | | | | | | | |
| 595 | DRB1_ | 820 | MIMRPE | 2 | 1209 | MRPEIK | 1 | Sequence | 0.812793 | 0.47 | <= |
| | 1303 | | IKNLED | | | NLE | | | | | SB |
| | | | KDN | | | | | | | | |
| 596 | DRB1_ | 821 | IMRPEI | 5 | 1210 | IKNLED | 0.993 | Sequence | 0.276816 | 4.43 | <= |
| | 0402 | | KNLEDK | | | KDN | | | | | WB |
| | | | DND | | | | | | | | |
| 596 | DRB1_ | 821 | IMRPEI | 5 | 1210 | IKNLED | 1 | Sequence | 0.412682 | 3.69 | <= |
| | 0404 | | KNLEDK | | | KDN | | | | | WB |
| | | | DND | | | | | | | | |
| 596 | DRB1_ | 821 | IMRPEI | 1 | 1209 | MRPEIK | 0.987 | Sequence | 0.428999 | 3.16 | <= |
| | 1303 | | KNLEDK | | | NLE | | | | | WB |
| | | | DND | | | | | | | | |
| 597 | DRB1_ | 822 | MRPEIK | 4 | 1210 | IKNLED | 1 | Sequence | 0.536123 | 1.32 | <= |
| | 0402 | | NLEDKD | | | KDN | | | | | WB |
| | | | NDK | | | | | | | | |
| 597 | DRB1_ | 822 | MRPEIK | 4 | 1210 | IKNLED | 0.993 | Sequence | 0.484472 | 3.16 | <= |
| | 0403 | | NLEDKD | | | KDN | | | | | WB |
| | | | NDK | | | | | | | | |
| 597 | DRB1_ | 822 | MRPEIK | 4 | 1210 | IKNLED | 1 | Sequence | 0.665766 | 1.39 | <= |
| | 0404 | | NLEDKD | | | KDN | | | | | WB |
| | | | NDK | | | | | | | | |
| 597 | DRB1_ | 822 | MRPEIK | 4 | 1210 | IKNLED | 0.98 | Sequence | 0.258102 | 4.75 | <= |
| | 0405 | | NLEDKD | | | KDN | | | | | WB |
| | | | NDK | | | | | | | | |
| 598 | DRB1_ | 823 | RPEIKN | 3 | 1210 | IKNLED | 1 | Sequence | 0.673505 | 0.64 | <= |
| | 0402 | | LEDKDN | | | KDN | | | | | SB |
| | | | DKK | | | | | | | | |
| 598 | DRB1_ | 823 | RPEIKN | 3 | 1210 | IKNLED | 0.987 | Sequence | 0.584999 | 1.97 | <= |
| | 0403 | | LEDKDN | | | KDN | | | | | WB |
| | | | DKK | | | | | | | | |
| 598 | DRB1_ | 823 | RPEIKN | 3 | 1210 | IKNLED | 1 | Sequence | 0.77014 | 0.81 | <= |
| | 0404 | | LEDKDN | | | KDN | | | | | SB |
| | | | DKK | | | | | | | | |
| 598 | DRB1_ | 823 | RPEIKN | 3 | 1210 | IKNLED | 1 | Sequence | 0.347712 | 3.27 | <= |
| | 0405 | | LEDKDN | | | KDN | | | | | WB |
| | | | DKK | | | | | | | | |
| 598 | DRB1_ | 823 | RPEIKN | 3 | 1210 | IKNLED | 1 | Sequence | 0.416296 | 3.47 | <= |
| | 0408 | | LEDKDN | | | KDN | | | | | WB |
| | | | DKK | | | | | | | | |
| 599 | DRB1_ | 824 | PEIKNL | 2 | 1210 | IKNLED | 0.967 | Sequence | 0.383377 | 2.68 | <= |
| | 0402 | | EDKDND | | | KDN | | | | | WB |
| | | | KKS | | | | | | | | |
| 599 | DRB1_ | 824 | PEIKNL | 2 | 1210 | IKNLED | 1 | Sequence | 0.493124 | 2.77 | <= |
| | 0404 | | EDKDND | | | KDN | | | | | WB |
| | | | KKS | | | | | | | | |
| 620 | DRB1_ | 825 | KKTTKL | 3 | 1211 | TKLTRD | 1 | Sequence | 0.243167 | 4.91 | <= |
| | 0701 | | TRDL YIPPE | | | LYI | | | | | WB |
| 631 | DRB1_ | 826 | IPPEPI | 5 | 1212 | ITLKNI | 0.953 | Sequence | 0.429598 | 2.16 | <= |
| | 0402 | | TLKNID | | | DQS | | | | | WB |
| | | | QSD | | | | | | | | |
| 631 | DRB1_ | 826 | IPPEPI | 5 | 1212 | ITLKNI | 0.827 | Sequence | 0.402185 | 4.48 | <= |
| | 0403 | | TLKNID | | | DOS | | | | | WB |
| | | | QSD | | | | | | | | |
| 632 | DRB1_ | 827 | PPEPIT | 4 | 1212 | ITLKNI | 0.967 | Sequence | 0.552301 | 1.23 | <= |
| | 0402 | | LKNIDO | | | DQS | | | | | WB |
| | | | SDS | | | | | | | | |
| 632 | DRB1_ | 827 | PPEPIT | 4 | 1212 | ITLKNI | 0.86 | Sequence | 0.508659 | 2.84 | <= |
| | 0403 | | LKNIDO | | | DQS | | | | | WB |
| | | | SDS | | | | | | | | |
| 633 | DRB1_ | 828 | PEPITL | 3 | 1212 | ITLKNI | 0.987 | Sequence | 0.55893 | 1.19 | <= |
| | 0402 | | KNIDQS | | | DOS | | | | | WB |
| | | | DSF | | | | | | | | |
| 633 | DRB1_ | 828 | PEPITL | 3 | 1212 | ITLKNI | 0.933 | Sequence | 0.510885 | 2.81 | <= |
| | 0403 | | KNIDQS | | | DOS | | | | | WB |
| | | | DSF | | | | | | | | |
| 634 | DRB1_ | 829 | EPITLK | 2 | 1212 | ITLKNI | 0.947 | Sequence | 0.339895 | 3.27 | <= |
| | 0402 | | NIDQSD | | | DQS | | | | | WB |
| | | | SFF | | | | | | | | |
| 636 | DRB1_ | 830 | ITLKNI | 5 | 1213 | IDQSDS | 1 | Sequence | 0.377105 | 2.84 | <= |
| | 0701 | | DQSDSF | | | FFI | | | | | WB |
| | | | FIR | | | | | | | | |
| 637 | DRB1_ | 831 | TLKNID | 4 | 1213 | IDQSDS | 1 | Sequence | 0.399055 | 2.61 | <= |
| | 0701 | | QSDSFF | | | FFI | | | | | WB |
| | | | IRF | | | | | | | | |
| 638 | DRB1_ | 832 | LKNIDO | 3 | 1213 | IDQSDS | 1 | Sequence | 0.494684 | 1.83 | <= |
| | 0701 | | SDSFFI | | | FFI | | | | | WB |
| | | | RFG | | | | | | | | |
| 638 | DRB1_ | 832 | LKNIDQ | 3 | 1213 | IDQSDS | 1 | Sequence | 0.356304 | 4.87 | <= |
| | 0901 | | SDSFFI | | | FFI | | | | | WB |
| | | | RFG | | | | | | | | |
| 638 | DRB1_ | 832 | LKNIDO | 3 | 1213 | IDQSDS | 1 | Sequence | 0.207056 | 4.83 | <= |
| | 1501 | | SDSFFI | | | FFI | | | | | WB |
| | | | RFG | | | | | | | | |
| 639 | DRB1_ | 833 | KNIDQS | 2 | 1213 | IDQSDS | 1 | Sequence | 0.356752 | 3.07 | <= |
| | 0701 | | DSFFIR | | | FFI | | | | | WB |
| | | | FGI | | | | | | | | |
| 644 | DRB1_ | 834 | SDSFFI | 4 | 1214 | FIRFGI | 0.94 | Sequence | 0.32026 | 3.73 | <= |
| | 1201 | | RFGINP | | | NPY | | | | | WB |
| | | | YLR | | | | | | | | |
| 644 | DRB1_ | 834 | SDSFFI | 5 | 1215 | IRFGIN | 0.62 | Sequence | 0.203021 | 4.92 | <= |
| | 1501 | | RFGINP | | | PYL | | | | | WB |
| | | | YLR | | | | | | | | |
| 644 | DRB1_ | 834 | SDSFFI | 5 | 1215 | IRFGIN | 0.48 | Sequence | 0.219762 | 3.66 | <= |
| | 1503 | | RFGINP | | | PYL | | | | | WB |
| | | | YLR | | | | | | | | |
| 645 | DRB1_ | 835 | DSFFIR | 3 | 1214 | FIRFGI | 0.9 | Sequence | 0.382147 | 2.63 | <= |
| | 1201 | | FGINPY | | | NPY | | | | | WB |
| | | | LRN | | | | | | | | |
| 645 | DRB1_ | 835 | DSFFIR | 4 | 1215 | IRFGIN | 0.74 | Sequence | 0.347446 | 2.81 | <= |
| | 1501 | | FGINPY | | | PYL | | | | | WB |
| | | | LRN | | | | | | | | |
| 645 | DRB1_ | 835 | DSFFIR | 4 | 1215 | IRFGIN | 0.547 | Sequence | 0.334041 | 2.04 | <= |
| | 1503 | | FGINPY | | | PYL | | | | | WB |
| | | | LRN | | | | | | | | |
| 645 | DRB1_ | 835 | DSFFIR | 3 | 1214 | FIRFGI | 0.567 | Sequence | 0.384686 | 2.56 | <= |
| | 1302 | | FGINPY | | | NPY | | | | | WB |
| | | | LRN | | | | | | | | |
| 646 | DRB1_ | 836 | SFFIRF | 3 | 1215 | IRFGIN | 0.9 | Sequence | 0.402118 | 2.34 | <= |
| | 1501 | | GINPYL | | | PYL | | | | | WB |
| | | | RNN | | | | | | | | |
| 646 | DRB1_ | 836 | SFFIRF | 3 | 1215 | IRFGIN | 0.84 | Sequence | 0.335765 | 2.02 | <= |
| | 1503 | | GINPYL | | | PYL | | | | | WB |
| | | | RNN | | | | | | | | |
| 646 | DRB1_ | 836 | SFFIRF | 3 | 1215 | IRFGIN | 0.54 | Sequence | 0.322954 | 4.14 | <= |
| | 1302 | | GINPYL | | | PYL | | | | | WB |
| | | | RNN | | | | | | | | |
| 647 | DRB1_ | 837 | FFIRFG | 2 | 1215 | IRFGIN | 0.76 | Sequence | 0.202401 | 4.93 | <= |
| | 1501 | | INPYLR | | | PYL | | | | | WB |
| | | | NNV | | | | | | | | |
| 647 | DRB1_ | 837 | FFIRFG | 2 | 1215 | IRFGIN | 0.613 | Sequence | 0.202434 | 4.04 | <= |
| | 1503 | | INPYLR | | | PYL | | | | | WB |
| | | | NNV | | | | | | | | |
| 648 | DRB1_ | 838 | FIRFGI | 5 | 1216 | INPYLR | 0.993 | Sequence | 0.768727 | 0.82 | <= |
| | 1301 | | NPYLRN | | | NNV | | | | | SB |
| | | | NVF | | | | | | | | |
| 648 | DRB1_ | 838 | FIRFGI | 5 | 1216 | INPYLR | 0.767 | Sequence | 0.229089 | 3.47 | <= |
| | 1503 | | NPYLRN | | | NNV | | | | | WB |
| | | | NVF | | | | | | | | |
| 648 | DRB1_ | 838 | FIRFGI | 5 | 1216 | INPYLR | 0.66 | Sequence | 0.355589 | 3.2 | <= |
| | 1302 | | NPYLRN | | | NNV | | | | | WB |
| | | | NVF | | | | | | | | |
| 649 | DRB1_ | 839 | IRFGIN | 4 | 1216 | INPYLR | 1 | Sequence | 0.823451 | 0.47 | <= |
| | 1301 | | PYLRNN | | | NNV | | | | | SB |
| | | | VFF | | | | | | | | |
| 649 | DRB1_ | 839 | IRFGIN | 4 | 1216 | INPYLR | 1 | Sequence | 0.199564 | 4.99 | <= |
| | 1501 | | PYLRNN | | | NNV | | | | | WB |
| | | | VFF | | | | | | | | |
| 649 | DRB1_ | 839 | IRFGIN | 4 | 1216 | INPYLR | 0.993 | Sequence | 0.252243 | 3.07 | <= |
| | 1503 | | PYLRNN | | | NNV | | | | | WB |
| | | | VFF | | | | | | | | |
| 649 | DRB1_ | 839 | IRFGIN | 4 | 1216 | INPYLR | 0.847 | Sequence | 0.381503 | 2.63 | <= |
| | 1302 | | PYLRNN | | | NNV | | | | | WB |
| | | | VFF | | | | | | | | |
| 650 | DRB1_ | 840 | RFGINP | 3 | 1216 | INPYLR | 1 | Sequence | 0.878742 | 0.21 | <= |
| | 1301 | | YLRNNV | | | NNV | | | | | SB |
| | | | FFD | | | | | | | | |
| 650 | DRB1_ | 840 | RFGINP | 3 | 1216 | INPYLR | 0.96 | Sequence | 0.369407 | 3.51 | <= |
| | 1401 | | YLRNNV | | | NNV | | | | | WB |
| | | | FFD | | | | | | | | |
| 650 | DRB1_ | 840 | RFGINP | 3 | 1216 | INPYLR | 1 | Sequence | 0.29049 | 3.42 | <= |
| | 1501 | | YLRNNV | | | NNV | | | | | WB |
| | | | FFD | | | | | | | | |
| 650 | DRB1_ | 840 | RFGINP | 3 | 1216 | INPYLR | 1 | Sequence | 0.331836 | 2.06 | <= |
| | 1503 | | YLRNNV | | | NNV | | | | | WB |
| | | | FFD | | | | | | | | |
| 650 | DRB1_ | 840 | RFGINP | 3 | 1216 | INPYLR | 0.96 | Sequence | 0.369407 | 3.51 | <= |
| | 1454 | | YLRNNV | | | NNV | | | | | WB |
| | | | FFD | | | | | | | | |
| 650 | DRB1_ | 840 | RFGINP | 3 | 1216 | INPYLR | 0.907 | Sequence | 0.462623 | 1.42 | <= |
| | 1302 | | YLRNNV | | | NNV | | | | | WB |
| | | | FFD | | | | | | | | |
| 651 | DRB1_ | 841 | FGINPY | 5 | 1217 | YLRNNV | 0.993 | Sequence | 0.320397 | 3.65 | <= |
| | 0405 | | LRNNVF | | | FFD | | | | | WB |
| | | | FDN | | | | | | | | |
| 651 | DRB1_ | 841 | FGINPY | 2 | 1216 | INPYLR | 0.993 | Sequence | 0.701121 | 1.31 | <= |
| | 1301 | | LRNNVF | | | NNV | | | | | WB |
| | | | FDN | | | | | | | | |
| 651 | DRB1_ | 841 | FGINPY | 2 | 1216 | INPYLR | 0.953 | Sequence | 0.176293 | 4.78 | <= |
| | 1503 | | LRNNVF | | | NNV | | | | | WB |
| | | | FDN | | | | | | | | |
| 651 | DRB1_ | 841 | FGINPY | 5 | 1217 | YLRNNV | 0.973 | Sequence | 0.45455 | 2.87 | <= |
| | 1303 | | LRNNVF | | | FFD | | | | | WB |
| | | | FDN | | | | | | | | |
| 651 | DRB1_ | 841 | FGINPY | 2 | 1216 | INPYLR | 0.613 | Sequence | 0.316093 | 4.34 | <= |
| | 1302 | | LRNNVF | | | NNV | | | | | WB |
| | | | FDN | | | | | | | | |
| 651 | DRB1_ | 841 | FGINPY | 5 | 1217 | YLRNNV | 0.807 | Sequence | 0.341597 | 3.34 | <= |
| | 0803 | | LRNNVF | | | FFD | | | | | WB |
| | | | FDN | | | | | | | | |
| 652 | DRB1_ | 842 | GINPYL | 1 | 1217 | YLRNNV | 1 | Sequence | 0.423162 | 2.46 | <= |
| | 0405 | | RNNVFF | | | FFD | | | | | WB |
| | | | DNY | | | | | | | | |
| 652 | DRB1_ | 842 | GINPYL | 4 | 1217 | YLRNNV | 0.973 | Sequence | 0.450452 | 4.44 | <= |
| | 0801 | | RNNVFF | | | FFD | | | | | WB |
| | | | DNY | | | | | | | | |
| 652 | DRB1_ | 842 | GINPYL | 4 | 1217 | YLRNNV | 0.913 | Sequence | 0.157645 | 4.88 | <= |
| | 1402 | | RNNVFF | | | FFD | | | | | WB |
| | | | DNY | | | | | | | | |
| 652 | DRB1_ | 842 | GINPYL | 4 | 1217 | YLRNNV | 1 | Sequence | 0.548867 | 1.93 | <= |
| | 1303 | | RNNVFF | | | FFD | | | | | WB |
| | | | DNY | | | | | | | | |
| 652 | DRB1_ | 842 | GINPYL | 4 | 1217 | YLRNNV | 0.913 | Sequence | 0.414604 | 2.02 | <= |
| | 0803 | | RNNVFF | | | FFD | | | | | WB |
| | | | DNY | | | | | | | | |
| 653 | DRB1_ | 843 | INPYLR | 3 | 1217 | YLRNNV | 1 | Sequence | 0.493777 | 1.91 | <= |
| | 0405 | | NNVFFD | | | FFD | | | | | WB |
| | | | NYG | | | | | | | | |
| 653 | DRB1_ | 843 | INPYLR | 3 | 1217 | YLRNNV | 0.993 | Sequence | 0.542213 | 2.93 | <= |
| | 0801 | | NNVFFD | | | FFD | | | | | WB |
| | | | NYG | | | | | | | | |
| 653 | DRB1_ | 843 | INPYLR | 3 | 1217 | YLRNNV | 0.947 | Sequence | 0.197734 | 3.17 | <= |
| | 1402 | | NNVFFD | | | FFD | | | | | WB |
| | | | NYG | | | | | | | | |
| 653 | DRB1_ | 843 | INPYLR | 3 | 1217 | YLRNNV | 1 | Sequence | 0.622104 | 1.4 | <= |
| | 1303 | | NNVFFD | | | FFD | | | | | WB |
| | | | NYG | | | | | | | | |
| 653 | DRB1_ | 843 | INPYLR | 3 | 1217 | YLRNNV | 0.96 | Sequence | 0.484953 | 1.24 | <= |
| | 0803 | | NNVFFD | | | FFD | | | | | WB |
| | | | NYG | | | | | | | | |
| 654 | DRB1_ | 844 | NPYLRN | 2 | 1217 | YLRNNV | 1 | Sequence | 0.294685 | 4.07 | <= |
| | 0405 | | NVFFDN | | | FFD | | | | | WB |
| | | | YGI | | | | | | | | |
| 654 | DRB1_ | 844 | NPYLRN | 2 | 1217 | YLRNNV | 1 | Sequence | 0.425544 | 3.2 | <= |
| | 1303 | | NVFFDN | | | FFD | | | | | WB |
| | | | YGI | | | | | | | | |
| 654 | DRB1_ | 844 | NPYLRN | 2 | 1217 | YLRNNV | 0.92 | Sequence | 0.317045 | 3.97 | <= |
| | 0803 | | NVFFDN | | | FFD | | | | | WB |
| | | | YGI | | | | | | | | |
| 656 | DRB1_ | 845 | YLRNNV | 4 | 1218 | NVFFDN | 0.987 | Sequence | 0.250258 | 3.97 | <= |
| | 1501 | | FFDNYG | | | YGI | | | | | WB |
| | | | ITS | | | | | | | | |
| 656 | DRB1_ | 845 | YLRNNV | 4 | 1218 | NVFFDN | 0.953 | Sequence | 0.19598 | 4.21 | <= |
| | 1503 | | FFDNYG | | | YGI | | | | | WB |
| | | | ITS | | | | | | | | |
| 657 | DRB1_ | 846 | LRNNVF | 3 | 1218 | NVFFDN | 0.987 | Sequence | 0.345122 | 2.83 | <= |
| | 1501 | | FDNYGI | | | YGI | | | | | WB |
| | | | TSP | | | | | | | | |
| 657 | DRB1_ | 846 | LRNNVF | 3 | 1218 | NVFFDN | 0.98 | Sequence | 0.273743 | 2.76 | <= |
| | 1503 | | FDNYGI | | | YGI | | | | | WB |
| | | | TSP | | | | | | | | |
| 658 | DRB1_ | 847 | RNNVFF | 2 | 1218 | NVFFDN | 0.967 | Sequence | 0.241867 | 4.13 | <= |
| | 1501 | | DNYGIT | | | YGI | | | | | WB |
| | | | SPK | | | | | | | | |
| 658 | DRB1_ | 847 | RNNVFF | 2 | 1218 | NVFFDN | 0.927 | Sequence | 0.191201 | 4.34 | <= |
| | 1503 | | DNYGIT | | | YGI | | | | | WB |
| | | | SPK | | | | | | | | |
| 659 | DRB1_ | 848 | NNVFFD | 3 | 1219 | FFDNYG | 0.893 | Sequence | 0.159166 | 4.79 | <= |
| | 1402 | | NYGITS | | | ITS | | | | | WB |
| | | | PKD | | | | | | | | |
| 661 | DRB1_ | 849 | VFFDNY | 5 | 1220 | YGITSP | 1 | Sequence | 0.407268 | 2.52 | <= |
| | 0701 | | GITSPK | | | KDF | | | | | WB |
| | | | DFN | | | | | | | | |
| 662 | DRB1_ | 850 | FFDNYG | 4 | 1220 | YGITSP | 1 | Sequence | 0.51228 | 1.69 | <= |
| | 0701 | | ITSPKD | | | KDF | | | | | WB |
| | | | FNY | | | | | | | | |
| 662 | DRB1_ | 850 | FFDNYG | 4 | 1220 | YGITSP | 0.993 | Sequence | 0.421411 | 3.46 | <= |
| | 0901 | | ITSPKD | | | KDF | | | | | WB |
| | | | FNY | | | | | | | | |
| 663 | DRB1_ | 851 | FDNYGI | 3 | 1220 | YGITSP | 1 | Sequence | 0.582791 | 1.27 | <= |
| | 0701 | | TSPKDF | | | KDF | | | | | WB |
| | | | NYQ | | | | | | | | |
| 663 | DRB1_ | 851 | FDNYGI | 3 | 1220 | YGITSP | 0.987 | Sequence | 0.48605 | 2.42 | <= |
| | 0901 | | TSPKDF | | | KDF | | | | | WB |
| | | | NYQ | | | | | | | | |
| 664 | DRB1_ | 852 | DNYGIT | 2 | 1220 | YGITSP | 0.993 | Sequence | 0.378302 | 2.83 | <= |
| | 0701 | | SPKDFN | | | KDF | | | | | WB |
| | | | YQI | | | | | | | | |
| 668 | DRB1_ | 853 | ITSPKD | 6 | 1221 | FNYQIK | 1 | Sequence | 0.325551 | 4.48 | <= |
| | 1401 | | FNYQIK | | | NYL | | | | | WB |
| | | | NYL | | | | | | | | |
| 668 | DRB1_ | 853 | ITSPKD | 6 | 1221 | FNYQIK | 1 | Sequence | 0.325551 | 4.48 | <= |
| | 1454 | | FNYQIK | | | NYL | | | | | WB |
| | | | NYL | | | | | | | | |
| 669 | DRB1_ | 854 | TSPKDF | 5 | 1221 | FNYQIK | 0.987 | Sequence | 0.472551 | 4.01 | <= |
| | 0801 | | NYQIKN | | | NYL | | | | | WB |
| | | | YLF | | | | | | | | |
| 669 | DRB1_ | 854 | TSPKDF | 5 | 1221 | FNYQIK | 1 | Sequence | 0.464389 | 3.46 | <= |
| | 1101 | | NYQIKN | | | NYL | | | | | WB |
| | | | YLF | | | | | | | | |
| 669 | DRB1_ | 854 | TSPKDF | 5 | 1221 | FNYQIK | 1 | Sequence | 0.489174 | 1.83 | <= |
| | 1401 | | NYQIKN | | | NYL | | | | | WB |
| | | | YLF | | | | | | | | |
| 669 | DRB1_ | 854 | TSPKDF | 5 | 1221 | FNYQIK | 1 | Sequence | 0.489174 | 1.83 | <= |
| | 1454 | | NYQIKN | | | NYL | | | | | WB |
| | | | YLF | | | | | | | | |
| 669 | DRB1_ | 854 | TSPKDF | 5 | 1221 | FNYQIK | 0.993 | Sequence | 0.26327 | 1.58 | <= |
| | 1402 | | NYQIKN | | | NYL | | | | | WB |
| | | | YLF | | | | | | | | |
| 669 | DRB1_ | 854 | TSPKDF | 5 | 1221 | FNYQIK | 0.993 | Sequence | 0.481397 | 2.59 | <= |
| | 1303 | | NYQIKN | | | NYL | | | | | WB |
| | | | YLF | | | | | | | | |
| 669 | DRB1_ | 854 | TSPKDF | 5 | 1221 | FNYQIK | 0.993 | Sequence | 0.365 | 2.96 | <= |
| | 1302 | | NYQIKN | | | NYL | | | | | WB |
| | | | YLF | | | | | | | | |
| 669 | DRB1_ | 854 | TSPKDF | 5 | 1221 | FNYQIK | 0.993 | Sequence | 0.372091 | 2.72 | <= |
| | 0803 | | NYQIKN | | | NYL | | | | | WB |
| | | | YLF | | | | | | | | |
| 670 | DRB1_ | 855 | SPKDFN | 4 | 1221 | FNYQIK | 0.967 | Sequence | 0.271472 | 4.29 | <= |
| | 0103 | | YQIKNY | | | NYL | | | | | WB |
| | | | LFD | | | | | | | | |
| 670 | DRB1_ | 855 | SPKDFN | 4 | 1221 | FNYQIK | 0.973 | Sequence | 0.131267 | 3.78 | <= |
| | 0305 | | YQIKNY | | | NYL | | | | | WB |
| | | | LFD | | | | | | | | |
| 670 | DRB1_ | 855 | SPKDFN | 4 | 1221 | FNYQIK | 0.967 | Sequence | 0.653273 | 1.69 | <= |
| | 0801 | | YQIKNY | | | NYL | | | | | WB |
| | | | LFD | | | | | | | | |
| 670 | DRB1_ | 855 | SPKDFN | 4 | 1221 | FNYQIK | 0.993 | Sequence | 0.691934 | 1.58 | <= |
| | 1101 | | YQIKNY | | | NYL | | | | | WB |
| | | | LFD | | | | | | | | |
| 670 | DRB1_ | 855 | SPKDFN | 4 | 1221 | FNYQIK | 0.993 | Sequence | 0.392919 | 4.95 | <= |
| | 1104 | | YQIKNY | | | NYL | | | | | WB |
| | | | LFD | | | | | | | | |
| 670 | DRB1_ | 855 | SPKDFN | 4 | 1221 | FNYQIK | 0.987 | Sequence | 0.45099 | 4.53 | <= |
| | 1301 | | YQIKNY | | | NYL | | | | | WB |
| | | | LFD | | | | | | | | |
| 670 | DRB1_ | 855 | SPKDFN | 4 | 1221 | FNYQIK | 1 | Sequence | 0.608942 | 0.9 | <= |
| | 1401 | | YQIKNY | | | NYL | | | | | SB |
| | | | LFD | | | | | | | | |
| 670 | DRB1_ | 855 | SPKDFN | 4 | 1221 | FNYQIK | 1 | Sequence | 0.608942 | 0.9 | <= |
| | 1454 | | YQIKNY | | | NYL | | | | | SB |
| | | | LFD | | | | | | | | |
| 670 | DRB1_ | 855 | SPKDFN | 4 | 1221 | FNYQIK | 1 | Sequence | 0.386728 | 0.47 | <= |
| | 1402 | | YQIKNY | | | NYL | | | | | SB |
| | | | LFD | | | | | | | | |
| 670 | DRB1_ | 855 | SPKDFN | 4 | 1221 | FNYQIK | 0.993 | Sequence | 0.662231 | 1.17 | <= |
| | 1303 | | YQIKNY | | | NYL | | | | | WB |
| | | | LFD | | | | | | | | |
| 670 | DRB1_ | 855 | SPKDFN | 4 | 1221 | FNYQIK | 1 | Sequence | 0.543251 | 0.69 | <= |
| | 1302 | | YQIKNY | | | NYL | | | | | SB |
| | | | LFD | | | | | | | | |
| 670 | DRB1_ | 855 | SPKDFN | 4 | 1221 | FNYQIK | 0.9 | Sequence | 0.54288 | 0.8 | <= |
| | 0803 | | YQIKNY | | | NYL | | | | | SB |
| | | | LFD | | | | | | | | |
| 671 | DRB1_ | 856 | PKDFNY | 3 | 1221 | FNYQIK | 0.953 | Sequence | 0.25302 | 4.65 | <= |
| | 0103 | | QIKNYL | | | NYL | | | | | WB |
| | | | FDI | | | | | | | | |
| 671 | DRB1_ | 856 | PKDFNY | 3 | 1221 | FNYQIK | 0.96 | Sequence | 0.125165 | 3.94 | <= |
| | 0305 | | QIKNYL | | | NYL | | | | | WB |
| | | | FDI | | | | | | | | |
| 671 | DRB1_ | 856 | PKDFNY | 3 | 1221 | FNYQIK | 0.833 | Sequence | 0.636189 | 1.85 | <= |
| | 0801 | | QIKNYL | | | NYL | | | | | WB |
| | | | FDI | | | | | | | | |
| 671 | DRB1_ | 856 | PKDFNY | 3 | 1221 | FNYQIK | 0.993 | Sequence | 0.649676 | 1.84 | <= |
| | 1101 | | QIKNYL | | | NYL | | | | | WB |
| | | | FDI | | | | | | | | |
| 671 | DRB1_ | 856 | PKDFNY | 3 | 1221 | FNYQIK | 0.98 | Sequence | 0.430303 | 4.93 | <= |
| | 1301 | | QIKNYL | | | NYL | | | | | WB |
| | | | FDI | | | | | | | | |
| 671 | DRB1_ | 856 | PKDFNY | 3 | 1221 | FNYQIK | 1 | Sequence | 0.597787 | 0.98 | <= |
| | 1401 | | QIKNYL | | | NYL | | | | | SB |
| | | | FDI | | | | | | | | |
| 671 | DRB1_ | 856 | PKDFNY | 3 | 1221 | FNYQIK | 1 | Sequence | 0.597787 | 0.98 | <= |
| | 1454 | | QIKNYL | | | NYL | | | | | SB |
| | | | FDI | | | | | | | | |
| 671 | DRB1_ | 856 | PKDFNY | 3 | 1221 | FNYQIK | 0.993 | Sequence | 0.376275 | 0.51 | <= |
| | 1402 | | QIKNYL | | | NYL | | | | | SB |
| | | | FDI | | | | | | | | |
| 671 | DRB1_ | 856 | PKDFNY | 3 | 1221 | FNYQIK | 0.987 | Sequence | 0.620375 | 1.41 | <= |
| | 1303 | | QIKNYL | | | NYL | | | | | WB |
| | | | FDI | | | | | | | | |
| 671 | DRB1_ | 856 | PKDFNY | 3 | 1221 | FNYQIK | 0.993 | Sequence | 0.526491 | 0.8 | <= |
| | 1302 | | QIKNYL | | | NYL | | | | | SB |
| | | | FDI | | | | | | | | |
| 671 | DRB1_ | 856 | PKDFNY | 3 | 1221 | FNYQIK | 0.72 | Sequence | 0.551595 | 0.76 | <= |
| | 0803 | | QIKNYL | | | NYL | | | | | SB |
| | | | FDI | | | | | | | | |
| 672 | DRB1_ | 857 | KDFNYQ | 4 | 1222 | YQIKNY | 0.607 | Sequence | 0.514398 | 3.34 | <= |
| | 0801 | | IKNYLF | | | LFD | | | | | WB |
| | | | DIK | | | | | | | | |
| 672 | DRB1_ | 857 | KDFNYQ | 2 | 1221 | FNYQIK | 0.953 | Sequence | 0.39251 | 3.07 | <= |
| | 1401 | | IKNYLF | | | NYL | | | | | WB |
| | | | DIK | | | | | | | | |
| 672 | DRB1_ | 857 | KDFNYQ | 2 | 1221 | FNYQIK | 0.953 | Sequence | 0.39251 | 3.07 | <= |
| | 1454 | | IKNYLF | | | NYL | | | | | WB |
| | | | DIK | | | | | | | | |
| 672 | DRB1_ | 857 | KDFNYQ | 2 | 1221 | FNYQIK | 0.88 | Sequence | 0.187599 | 3.53 | <= |
| | 1402 | | IKNYLF | | | NYL | | | | | WB |
| | | | DIK | | | | | | | | |
| 672 | DRB1_ | 857 | KDFNYQ | 2 | 1221 | FNYQIK | 0.827 | Sequence | 0.327488 | 4.57 | <= |
| | 1303 | | IKNYLF | | | NYL | | | | | WB |
| | | | DIK | | | | | | | | |
| 672 | DRB1_ | 857 | KDFNYQ | 4 | 1222 | YQIKNY | 0.573 | Sequence | 0.477018 | 1.31 | <= |
| | 0803 | | IKNYLF | | | LFD | | | | | WB |
| | | | DIK | | | | | | | | |
| 673 | DRB1_ | 858 | DFNYQI | 3 | 1222 | YQIKNY | 0.887 | Sequence | 0.499509 | 3.57 | <= |
| | 0801 | | KNYLFD | | | LFD | | | | | WB |
| | | | IKN | | | | | | | | |
| 673 | DRB1_ | 858 | DFNYQI | 3 | 1222 | YQIKNY | 0.833 | Sequence | 0.448544 | 1.59 | <= |
| | 0803 | | KNYLFD | | | LFD | | | | | WB |
| | | | IKN | | | | | | | | |
| 675 | DRB1_ | 859 | NYQIKN | 6 | 1223 | YLFDIK | 0.993 | Sequence | 0.225391 | 4.94 | <= |
| | 0301 | | YLFDIK | | | NKT | | | | | WB |
| | | | NKT | | | | | | | | |
| 675 | DRB1_ | 859 | NYQIKN | 6 | 1223 | YLFDIK | 0.987 | Sequence | 0.32413 | 1.29 | <= |
| | 0305 | | YLFDIK | | | NKT | | | | | WB |
| | | | NKT | | | | | | | | |
| 675 | DRB1_ | 859 | NYQIKN | 6 | 1223 | YLFDIK | 0.94 | Sequence | 0.176673 | 3.95 | <= |
| | 1402 | | YLFDIK | | | NKT | | | | | WB |
| | | | NKT | | | | | | | | |
| 675 | DRB1_ | 859 | NYQIKN | 6 | 1223 | YLFDIK | 1 | Sequence | 0.45438 | 2.87 | <= |
| | 1303 | | YLFDIK | | | NKT | | | | | WB |
| | | | NKT | | | | | | | | |
| 676 | DRB1_ | 860 | YQIKNY | 5 | 1223 | YLFDIK | 1 | Sequence | 0.518637 | 1.91 | <= |
| | 0301 | | LFDIKN | | | NKT | | | | | WB |
| | | | KTD | | | | | | | | |
| 676 | DRB1_ | 860 | YQIKNY | 5 | 1223 | YLFDIK | 1 | Sequence | 0.640998 | 0.21 | <= |
| | 0305 | | LFDIKN | | | NKT | | | | | SB |
| | | | KTD | | | | | | | | |
| 676 | DRB1_ | 860 | YQIKNY | 5 | 1223 | YLFDIK | 0.92 | Sequence | 0.534484 | 3.04 | <= |
| | 0801 | | LFDIKN | | | NKT | | | | | WB |
| | | | KTD | | | | | | | | |
| 676 | DRB1_ | 860 | YQIKNY | 5 | 1223 | YLFDIK | 1 | Sequence | 0.659286 | 1.77 | <= |
| | 1101 | | LFDIKN | | | NKT | | | | | WB |
| | | | KTD | | | | | | | | |
| 676 | DRB1_ | 860 | YQIKNY | 5 | 1223 | YLFDIK | 0.993 | Sequence | 0.46034 | 0.22 | <= |
| | 1402 | | LFDIKN | | | NKT | | | | | SB |
| | | | KTD | | | | | | | | |
| 676 | DRB1_ | 860 | YQIKNY | 5 | 1223 | YLFDIK | 1 | Sequence | 0.775177 | 0.62 | <= |
| | 1303 | | LFDIKN | | | NKT | | | | | SB |
| | | | KTD | | | | | | | | |
| 676 | DRB1_ | 860 | YQIKNY | 5 | 1223 | YLFDIK | 0.973 | Sequence | 0.397774 | 2.32 | <= |
| | 1302 | | LFDIKN | | | NKT | | | | | WB |
| | | | KTD | | | | | | | | |
| 676 | DRB1_ | 860 | YQIKNY | 5 | 1223 | YLFDIK | 0.92 | Sequence | 0.408072 | 2.13 | <= |
| | 0803 | | LFDIKN | | | NKT | | | | | WB |
| | | | KTD | | | | | | | | |
| 677 | DRB1_ | 861 | QIKNYL | 4 | 1223 | YLFDIK | 1 | Sequence | 0.558865 | 1.69 | <= |
| | 0301 | | FDIKNK | | | NKT | | | | | WB |
| | | | TDI | | | | | | | | |
| 677 | DRB1_ | 861 | QIKNYL | 4 | 1223 | YLFDIK | 1 | Sequence | 0.682342 | 0.15 | <= |
| | 0305 | | FDIKNK | | | NKT | | | | | SB |
| | | | TDI | | | | | | | | |
| 677 | DRB1_ | 861 | QIKNYL | 5 | 1224 | LFDIKN | 0.913 | Sequence | 0.267411 | 4.56 | <= |
| | 0405 | | FDIKNK | | | KTD | | | | | WB |
| | | | TDI | | | | | | | | |
| 677 | DRB1_ | 861 | QIKNYL | 4 | 1223 | YLFDIK | 0.747 | Sequence | 0.606678 | 2.16 | <= |
| | 0801 | | FDIKNK | | | NKT | | | | | WB |
| | | | TDI | | | | | | | | |
| 677 | DRB1_ | 861 | QIKNYL | 4 | 1223 | YLFDIK | 1 | Sequence | 0.727654 | 1.37 | <= |
| | 1101 | | FDIKNK | | | NKT | | | | | WB |
| | | | TDI | | | | | | | | |
| 677 | DRB1_ | 861 | QIKNYL | 4 | 1223 | YLFDIK | 0.987 | Sequence | 0.511474 | 0.12 | <= |
| | 1402 | | FDIKNK | | | NKT | | | | | SB |
| | | | TDI | | | | | | | | |
| 677 | DRB1_ | 861 | QIKNYL | 4 | 1223 | YLFDIK | 1 | Sequence | 0.791076 | 0.56 | <= |
| | 1303 | | FDIKNK | | | NKT | | | | | SB |
| | | | TDI | | | | | | | | |
| 677 | DRB1_ | 861 | QIKNYL | 4 | 1223 | YLFDIK | 0.913 | Sequence | 0.468487 | 1.35 | <= |
| | 1302 | | FDIKNK | | | NKT | | | | | WB |
| | | | TDI | | | | | | | | |
| 677 | DRB1_ | 861 | QIKNYL | 4 | 1223 | YLFDIK | 0.807 | Sequence | 0.465656 | 1.42 | <= |
| | 0803 | | FDIKNK | | | NKT | | | | | WB |
| | | | TDI | | | | | | | | |
| 678 | DRB1_ | 862 | IKNYLF | 3 | 1223 | YLFDIK | 1 | Sequence | 0.61961 | 1.36 | <= |
| | 0301 | | DIKNKT | | | NKT | | | | | WB |
| | | | DIK | | | | | | | | |
| 678 | DRB1_ | 862 | IKNYLF | 3 | 1223 | YLFDIK | 1 | Sequence | 0.734822 | 0.09 | <= |
| | 0305 | | DIKNKT | | | NKT | | | | | SB |
| | | | DIK | | | | | | | | |
| 678 | DRB1_ | 862 | IKNYLF | 4 | 1224 | LFDIKN | 0.827 | Sequence | 0.254955 | 4.93 | <= |
| | 0402 | | DIKNKT | | | KTD | | | | | WB |
| | | | DIK | | | | | | | | |
| 678 | DRB1_ | 862 | IKNYLF | 4 | 1224 | LFDIKN | 0.84 | Sequence | 0.445754 | 3.7 | <= |
| | 0403 | | DIKNKT | | | KTD | | | | | WB |
| | | | DIK | | | | | | | | |
| 678 | DRB1_ | 862 | IKNYLF | 4 | 1224 | LFDIKN | 0.92 | Sequence | 0.474199 | 2.04 | <= |
| | 0405 | | DIKNKT | | | KTD | | | | | WB |
| | | | DIK | | | | | | | | |
| 678 | DRB1_ | 862 | IKNYLF | 3 | 1223 | YLFDIK | 0.66 | Sequence | 0.735496 | 1.03 | <= |
| | 0801 | | DIKNKT | | | NKT | | | | | WB |
| | | | DIK | | | | | | | | |
| 678 | DRB1_ | 862 | IKNYLF | 3 | 1223 | YLFDIK | 0.993 | Sequence | 0.826908 | 0.8 | <= |
| | 1101 | | DIKNKT | | | NKT | | | | | SB |
| | | | DIK | | | | | | | | |
| 678 | DRB1_ | 862 | IKNYLF | 3 | 1223 | YLFDIK | 0.867 | Sequence | 0.444883 | 4.16 | <= |
| | 1104 | | DIKNKT | | | NKT | | | | | WB |
| | | | DIK | | | | | | | | |
| 678 | DRB1_ | 862 | IKNYLF | 3 | 1223 | YLFDIK | 0.82 | Sequence | 0.342551 | 4.07 | <= |
| | 1401 | | DIKNKT | | | NKT | | | | | WB |
| | | | DIK | | | | | | | | |
| 678 | DRB1_ | 862 | IKNYLF | 3 | 1223 | YLFDIK | 0.82 | Sequence | 0.342551 | 4.07 | <= |
| | 1454 | | DIKNKT | | | NKT | | | | | WB |
| | | | DIK | | | | | | | | |
| 678 | DRB1_ | 862 | IKNYLF | 3 | 1223 | YLFDIK | 0.98 | Sequence | 0.592151 | 0.04 | <= |
| | 1402 | | DIKNKT | | | NKT | | | | | SB |
| | | | DIK | | | | | | | | |
| 678 | DRB1_ | 862 | IKNYLF | 3 | 1223 | YLFDIK | 0.993 | Sequence | 0.8376 | 0.38 | <= |
| | 1303 | | DIKNKT | | | NKT | | | | | SB |
| | | | DIK | | | | | | | | |
| 678 | DRB1_ | 862 | IKNYLF | 3 | 1223 | YLFDIK | 0.867 | Sequence | 0.602112 | 0.38 | <= |
| | 1302 | | DIKNKT | | | NKT | | | | | SB |
| | | | DIK | | | | | | | | |
| 678 | DRB1_ | 862 | IKNYLF | 3 | 1223 | YLFDIK | 0.76 | Sequence | 0.574032 | 0.63 | <= |
| | 0803 | | DIKNKT | | | NKT | | | | | SB |
| | | | DIK | | | | | | | | |
| 679 | DRB1_ | 863 | KNYLFD | 2 | 1223 | YLFDIK | 0.993 | Sequence | 0.506555 | 1.99 | <= |
| | 0301 | | IKNKTD | | | NKT | | | | | WB |
| | | | IKI | | | | | | | | |
| 679 | DRB1_ | 863 | KNYLFD | 2 | 1223 | YLFDIK | 1 | Sequence | 0.630442 | 0.22 | <= |
| | 0305 | | IKNKTD | | | NKT | | | | | SB |
| | | | IKI | | | | | | | | |
| 679 | DRB1_ | 863 | KNYLFD | 3 | 1224 | LFDIKN | 0.933 | Sequence | 0.41451 | 4.25 | <= |
| | 0403 | | IKNKTD | | | KTD | | | | | WB |
| | | | IKI | | | | | | | | |
| 679 | DRB1_ | 863 | KNYLFD | 3 | 1224 | LFDIKN | 0.987 | Sequence | 0.447261 | 2.26 | <= |
| | 0405 | | IKNKTD | | | KTD | | | | | WB |
| | | | IKI | | | | | | | | |
| 679 | DRB1_ | 863 | KNYLFD | 2 | 1223 | YLFDIK | 0.493 | Sequence | 0.637774 | 1.84 | <= |
| | 0801 | | IKNKTD | | | NKT | | | | | WB |
| | | | IKI | | | | | | | | |
| 679 | DRB1_ | 863 | KNYLFD | 2 | 1223 | YLFDIK | 0.96 | Sequence | 0.688902 | 1.59 | <= |
| | 1101 | | IKNKTD | | | NKT | | | | | WB |
| | | | IKI | | | | | | | | |
| 679 | DRB1_ | 863 | KNYLFD | 2 | 1223 | YLFDIK | 0.967 | Sequence | 0.456853 | 0.23 | <= |
| | 1402 | | IKNKTD | | | NKT | | | | | SB |
| | | | IKI | | | | | | | | |
| 679 | DRB1_ | 863 | KNYLFD | 2 | 1223 | YLFDIK | 0.947 | Sequence | 0.737505 | 0.78 | <= |
| | 1303 | | IKNKTD | | | NKT | | | | | SB |
| | | | IKI | | | | | | | | |
| 679 | DRB1_ | 863 | KNYLFD | 2 | 1223 | YLFDIK | 0.64 | Sequence | 0.510796 | 0.93 | <= |
| | 1302 | | IKNKTD | | | NKT | | | | | SB |
| | | | IKI | | | | | | | | |
| 679 | DRB1_ | 863 | KNYLFD | 2 | 1223 | YLFDIK | 0.56 | Sequence | 0.450944 | 1.56 | <= |
| | 0803 | | IKNKTD | | | NKT | | | | | WB |
| | | | IKI | | | | | | | | |
| 680 | DRB1_ | 864 | NYLFDI | 1 | 1223 | YLFDIK | 0.967 | Sequence | 0.283651 | 1.58 | <= |
| | 0305 | | KNKTDI | | | NKT | | | | | WB |
| | | | KIH | | | | | | | | |
| 680 | DRB1_ | 864 | NYLFDI | 5 | 1225 | IKNKTD | 0.853 | Sequence | 0.291014 | 4.12 | <= |
| | 0402 | | KNKTDI | | | IKI | | | | | WB |
| | | | KIH | | | | | | | | |
| 680 | DRB1_ | 864 | NYLFDI | 1 | 1223 | YLFDIK | 0.693 | Sequence | 0.157694 | 4.87 | <= |
| | 1402 | | KNKTDI | | | NKT | | | | | WB |
| | | | KIH | | | | | | | | |
| 680 | DRB1_ | 864 | NYLFDI | 1 | 1223 | YLFDIK | 0.733 | Sequence | 0.349385 | 4.22 | <= |
| | 1303 | | KNKTDI | | | NKT | | | | | WB |
| | | | KIH | | | | | | | | |
| 680 | DRB1_ | 864 | NYLFDI | 3 | 1226 | FDIKNK | 0.64 | Sequence | 0.363794 | 2.98 | <= |
| | 1302 | | KNKTDI | | | TDI | | | | | WB |
| | | | KIH | | | | | | | | |
| 681 | DRB1_ | 865 | YLFDIK | 4 | 1225 | IKNKTD | 0.993 | Sequence | 0.442569 | 2.03 | <= |
| | 0402 | | NKTDIK | | | IKI | | | | | WB |
| | | | IHA | | | | | | | | |
| 681 | DRB1_ | 865 | YLFDIK | 4 | 1225 | IKNKTD | 1 | Sequence | 0.233486 | 4.3 | <= |
| | 1501 | | NKTDIK | | | IKI | | | | | WB |
| | | | IHA | | | | | | | | |
| 681 | DRB1_ | 865 | YLFDIK | 4 | 1225 | IKNKTD | 0.98 | Sequence | 0.196725 | 4.19 | <= |
| | 1503 | | NKTDIK | | | IKI | | | | | WB |
| | | | IHA | | | | | | | | |
| 682 | DRB1_ | 866 | LFDIKN | 3 | 1225 | IKNKTD | 1 | Sequence | 0.559426 | 1.19 | <= |
| | 0402 | | KTDIKI | | | IKI | | | | | WB |
| | | | HAD | | | | | | | | |
| 682 | DRB1_ | 866 | LFDIKN | 3 | 1225 | IKNKTD | 0.987 | Sequence | 0.460244 | 4.36 | <= |
| | 1301 | | KTDIKI | | | IKI | | | | | WB |
| | | | HAD | | | | | | | | |
| 682 | DRB1_ | 866 | LFDIKN | 3 | 1225 | IKNKTD | 1 | Sequence | 0.351167 | 2.77 | <= |
| | 1501 | | KTDIKI | | | IKI | | | | | WB |
| | | | HAD | | | | | | | | |
| 682 | DRB1_ | 866 | LFDIKN | 3 | 1225 | IKNKTD | 0.993 | Sequence | 0.27104 | 2.8 | <= |
| | 1503 | | KTDIKI | | | IKI | | | | | WB |
| | | | HAD | | | | | | | | |
| 683 | DRB1_ | 867 | FDIKNK | 2 | 1225 | IKNKTD | 0.993 | Sequence | 0.403816 | 2.44 | <= |
| | 0402 | | TDIKIH | | | IKI | | | | | WB |
| | | | ADF | | | | | | | | |
| 683 | DRB1_ | 867 | FDIKNK | 2 | 1225 | IKNKTD | 0.993 | Sequence | 0.208555 | 4.8 | <= |
| | 1501 | | TDIKIH | | | IKI | | | | | WB |
| | | | ADF | | | | | | | | |
| 683 | DRB1_ | 867 | FDIKNK | 2 | 1225 | IKNKTD | 1 | Sequence | 0.18372 | 4.55 | <= |
| | 1503 | | TDIKIH | | | IKI | | | | | WB |
| | | | ADF | | | | | | | | |
| 684 | DRB1_ | 868 | DIKNKT | 3 | 1227 | NKTDIK | 0.96 | Sequence | 0.244181 | 4.62 | <= |
| | 0301 | | DIKIHA | | | IHA | | | | | WB |
| | | | DFY | | | | | | | | |
| 686 | DRB1_ | 869 | KNKTDI | 5 | 1228 | IKIHAD | 1 | Sequence | 0.728332 | 0.44 | <= |
| | 0402 | | KIHADF | | | FYN | | | | | SB |
| | | | YNR | | | | | | | | |
| 686 | DRB1_ | 869 | KNKTDI | 5 | 1228 | IKIHAD | 0.913 | Sequence | 0.442348 | 4.7 | <= |
| | 1301 | | KIHADF | | | FYN | | | | | WB |
| | | | YNR | | | | | | | | |
| 686 | DRB1_ | 869 | KNKTDI | 5 | 1228 | IKIHAD | 0.993 | Sequence | 0.561739 | 1.38 | <= |
| | 1501 | | KIHADF | | | FYN | | | | | WB |
| | | | YNR | | | | | | | | |
| 686 | DRB1_ | 869 | KNKTDI | 5 | 1228 | IKIHAD | 0.993 | Sequence | 0.435664 | 1.29 | <= |
| | 1503 | | KIHADF | | | FYN | | | | | WB |
| | | | YNR | | | | | | | | |
| 687 | DRB1_ | 870 | NKTDIK | 4 | 1228 | IKIHAD | 1 | Sequence | 0.768273 | 0.32 | <= |
| | 0402 | | IHADFY | | | FYN | | | | | SB |
| | | | NRL | | | | | | | | |
| 687 | DRB1_ | 870 | NKTDIK | 4 | 1228 | IKIHAD | 1 | Sequence | 0.380637 | 4.13 | <= |
| | 0404 | | IHADFY | | | FYN | | | | | WB |
| | | | NRL | | | | | | | | |
| 687 | DRB1_ | 870 | NKTDIK | 4 | 1228 | IKIHAD | 0.967 | Sequence | 0.482633 | 3.96 | <= |
| | 1301 | | IHADFY | | | FYN | | | | | WB |
| | | | NRL | | | | | | | | |
| 687 | DRB1_ | 870 | NKTDIK | 4 | 1228 | IKIHAD | 1 | Sequence | 0.607093 | 1.17 | <= |
| | 1501 | | IHADFY | | | FYN | | | | | WB |
| | | | NRL | | | | | | | | |
| 687 | DRB1_ | 870 | NKTDIK | 4 | 1228 | IKIHAD | 0.987 | Sequence | 0.480475 | 1.05 | <= |
| | 1503 | | IHADFY | | | FYN | | | | | WB |
| | | | NRL | | | | | | | | |
| 688 | DRB1_ | 871 | KTDIKI | 5 | 1229 | IHADFY | 0.893 | Sequence | 0.120855 | 4.07 | <= |
| | 0305 | | HADFYN | | | NRL | | | | | WB |
| | | | RLI | | | | | | | | |
| 688 | DRB1_ | 871 | KTDIKI | 3 | 1228 | IKIHAD | 1 | Sequence | 0.757761 | 0.35 | <= |
| | 0402 | | HADFYN | | | FYN | | | | | SB |
| | | | RLI | | | | | | | | |
| 688 | DRB1_ | 871 | KTDIKI | 3 | 1228 | IKIHAD | 0.993 | Sequence | 0.371629 | 4.26 | <= |
| | 0404 | | HADFYN | | | FYN | | | | | WB |
| | | | RLI | | | | | | | | |
| 688 | DRB1_ | 871 | KTDIKI | 3 | 1228 | IKIHAD | 0.907 | Sequence | 0.489662 | 3.84 | <= |
| | 1301 | | HADFYN | | | FYN | | | | | WB |
| | | | RLI | | | | | | | | |
| 688 | DRB1_ | 871 | KTDIKI | 3 | 1228 | IKIHAD | 0.993 | Sequence | 0.597536 | 1.21 | <= |
| | 1501 | | HADFYN | | | FYN | | | | | WB |
| | | | RLI | | | | | | | | |
| 688 | DRB1_ | 871 | KTDIKI | 3 | 1228 | IKIHAD | 0.973 | Sequence | 0.474944 | 1.08 | <= |
| | 1503 | | HADFYN | | | FYN | | | | | WB |
| | | | RLI | | | | | | | | |
| 688 | DRB1_ | 871 | KTDIKI | 3 | 1228 | IKIHAD | 0.533 | Sequence | 0.319432 | 4.24 | <= |
| | 1302 | | HADFYN | | | FYN | | | | | WB |
| | | | RLI | | | | | | | | |
| 689 | DRB1_ | 872 | TDIKIH | 4 | 1229 | IHADFY | 0.967 | Sequence | 0.232421 | 4.82 | <= |
| | 0301 | | ADFYNR | | | NRL | | | | | WB |
| | | | LIT | | | | | | | | |
| 689 | DRB1_ | 872 | TDIKIH | 4 | 1229 | IHADFY | 0.96 | Sequence | 0.156113 | 3.18 | <= |
| | 0305 | | ADFYNR | | | NRL | | | | | WB |
| | | | LIT | | | | | | | | |
| 689 | DRB1_ | 872 | TDIKIH | 2 | 1228 | IKIHAD | 1 | Sequence | 0.461167 | 1.87 | <= |
| | 0402 | | ADFYNR | | | FYN | | | | | WB |
| | | | LIT | | | | | | | | |
| 689 | DRB1_ | 872 | TDIKIH | 2 | 1228 | IKIHAD | 0.94 | Sequence | 0.304566 | 3.26 | <= |
| | 1501 | | ADFYNR | | | FYN | | | | | WB |
| | | | LIT | | | | | | | | |
| 689 | DRB1_ | 872 | TDIKIH | 2 | 1228 | IKIHAD | 0.873 | Sequence | 0.227287 | 3.51 | <= |
| | 1503 | | ADFYNR | | | FYN | | | | | WB |
| | | | LIT | | | | | | | | |
| 690 | DRB1_ | 873 | DIKIHA | 3 | 1229 | IHADFY | 0.987 | Sequence | 0.22273 | 4.99 | <= |
| | 0301 | | DFYNRL | | | NRL | | | | | WB |
| | | | ITF | | | | | | | | |
| 690 | DRB1_ | 873 | DIKIHA | 3 | 1229 | IHADFY | 0.973 | Sequence | 0.14932 | 3.33 | <= |
| | 0305 | | DFYNRL | | | NRL | | | | | WB |
| | | | ITF | | | | | | | | |
| 693 | DRB1_ | 874 | IHADFY | 5 | 1230 | YNRLIT | 0.987 | Sequence | 0.479399 | 3.29 | <= |
| | 1001 | | NRLITF | | | FEN | | | | | WB |
| | | | ENL | | | | | | | | |
| 694 | DRB1_ | 875 | HADFYN | 4 | 1230 | YNRLIT | 0.98 | Sequence | 0.484108 | 3.23 | <= |
| | 1001 | | RLITFE | | | FEN | | | | | WB |
| | | | NLL | | | | | | | | |
| 695 | DRB1_ | 876 | ADFYNR | 3 | 1230 | YNRLIT | 0.993 | Sequence | 0.504863 | 2.94 | <= |
| | 1001 | | LITFEN | | | FEN | | | | | WB |
| | | | LLY | | | | | | | | |
| 697 | DRB1_ | 877 | FYNRLI | 4 | 1231 | LITFEN | 0.873 | Sequence | 0.355103 | 3.06 | <= |
| | 1201 | | TFENLL | | | LLY | | | | | WB |
| | | | YLN | | | | | | | | |
| 697 | DRB1_ | 877 | FYNRLI | 4 | 1231 | LITFEN | 0.987 | Sequence | 0.259115 | 3.84 | <= |
| | 1501 | | TFENLL | | | LLY | | | | | WB |
| | | | YLN | | | | | | | | |
| 697 | DRB1_ | 877 | FYNRLI | 4 | 1231 | LITFEN | 0.967 | Sequence | 0.193665 | 4.28 | <= |
| | 1503 | | TFENLL | | | LLY | | | | | WB |
| | | | YLN | | | | | | | | |
| 698 | DRB1_ | 878 | YNRLIT | 3 | 1231 | LITFEN | 0.86 | Sequence | 0.39712 | 2.41 | <= |
| | 1201 | | FENLLY | | | LLY | | | | | WB |
| | | | LNT | | | | | | | | |
| 698 | DRB1_ | 878 | YNRLIT | 3 | 1231 | LITFEN | 0.987 | Sequence | 0.295596 | 3.36 | <= |
| | 1501 | | FENLLY | | | LLY | | | | | WB |
| | | | LNT | | | | | | | | |
| 698 | DRB1_ | 878 | YNRLIT | 3 | 1231 | LITFEN | 0.953 | Sequence | 0.21794 | 3.7 | <= |
| | 1503 | | FENLLY | | | LLY | | | | | WB |
| | | | LNT | | | | | | | | |
| 699 | DRB1_ | 879 | NRLITF | 2 | 1231 | LITFEN | 0.847 | Sequence | 0.296476 | 4.29 | <= |
| | 1201 | | ENLLYL | | | LLY | | | | | WB |
| | | | NTI | | | | | | | | |
| 701 | DRB1_ | 880 | LITFEN | 6 | 1232 | LLYLNT | 0.993 | Sequence | 0.489234 | 1.44 | <= |
| | 1201 | | LLYLNT | | | IEY | | | | | WB |
| | | | IEY | | | | | | | | |
| 702 | DRB1_ | 881 | ITFENL | 5 | 1232 | LLYLNT | 1 | Sequence | 0.797803 | 0.15 | <= |
| | 1201 | | LYLNTI | | | IEY | | | | | SB |
| | | | EYS | | | | | | | | |
| 703 | DRB1_ | 882 | TFENLL | 4 | 1232 | LLYLNT | 1 | Sequence | 0.890832 | 0.03 | <= |
| | 1201 | | YLNTIE | | | IEY | | | | | SB |
| | | | YSP | | | | | | | | |
| 704 | DRB1_ | 883 | FENLLY | 3 | 1232 | LLYLNT | 1 | Sequence | 0.853039 | 0.07 | <= |
| | 1201 | | LNTIEY | | | IEY | | | | | SB |
| | | | SPL | | | | | | | | |
| 705 | DRB1_ | 884 | ENLLYL | 2 | 1232 | LLYLNT | 1 | Sequence | 0.80854 | 0.14 | <= |
| | 1201 | | NTIEYS | | | IEY | | | | | SB |
| | | | PLN | | | | | | | | |
| 706 | DRB1_ | 885 | NLLYLN | 1 | 1232 | LLYLNT | 0.867 | Sequence | 0.444658 | 1.84 | <= |
| | 1201 | | TIEYSP | | | IEY | | | | | WB |
| | | | LNK | | | | | | | | |
| 709 | DRB1_ | 886 | YLNTIE | 4 | 1233 | IEYSPL | 0.973 | Sequence | 0.363365 | 4.01 | <= |
| | 1303 | | YSPLNK | | | NKD | | | | | WB |
| | | | DFK | | | | | | | | |
| 709 | DRB1_ | 886 | YLNTIE | 4 | 1233 | IEYSPL | 0.927 | Sequence | 0.359645 | 2.94 | <= |
| | 0803 | | YSPLNK | | | NKD | | | | | WB |
| | | | DFK | | | | | | | | |
| 710 | DRB1_ | 887 | LNTIEY | 3 | 1233 | IEYSPL | 0.88 | Sequence | 0.385974 | 3.69 | <= |
| | 1303 | | SPLNKD | | | NKD | | | | | WB |
| | | | FKV | | | | | | | | |
| 710 | DRB1_ | 887 | LNTIEY | 3 | 1233 | IEYSPL | 0.92 | Sequence | 0.382795 | 2.53 | <= |
| | 0803 | | SPLNKD | | | NKD | | | | | WB |
| | | | FKV | | | | | | | | |
| 711 | DRB1_ | 888 | NTIEYS | 4 | 1234 | YSPLNK | 0.973 | Sequence | 0.358817 | 4.8 | <= |
| | 1101 | | PLNKDF | | | DFK | | | | | WB |
| | | | KVE | | | | | | | | |
| 711 | DRB1_ | 888 | NTIEYS | 2 | 1233 | IEYSPL | 0.653 | Sequence | 0.313122 | 4.83 | <= |
| | 1303 | | PLNKDF | | | NKD | | | | | WB |
| | | | KVE | | | | | | | | |
| 711 | DRB1_ | 888 | NTIEYS | 2 | 1233 | IEYSPL | 0.753 | Sequence | 0.294961 | 4.64 | <= |
| | 0803 | | PLNKDF | | | NKD | | | | | WB |
| | | | KVE | | | | | | | | |
| 712 | DRB1_ | 889 | TIEYSP | 3 | 1234 | YSPLNK | 0.993 | Sequence | 0.477918 | 3.31 | <= |
| | 1101 | | LNKDFK | | | DFK | | | | | WB |
| | | | VED | | | | | | | | |
| 712 | DRB1_ | 889 | TIEYSP | 6 | 1235 | LNKDFK | 0.513 | Sequence | 0.405098 | 3.44 | <= |
| | 1303 | | LNKDFK | | | VED | | | | | WB |
| | | | VED | | | | | | | | |
| 713 | DRB1_ | 890 | IEYSPL | 5 | 1235 | LNKDFK | 0.993 | Sequence | 0.512668 | 1.95 | <= |
| | 0301 | | NKDFKV | | | VED | | | | | WB |
| | | | EDK | | | | | | | | |
| 713 | DRB1_ | 890 | IEYSPL | 5 | 1235 | LNKDFK | 0.92 | Sequence | 0.226243 | 2.17 | <= |
| | 0305 | | NKDFKV | | | VED | | | | | WB |
| | | | EDK | | | | | | | | |
| 713 | DRB1_ | 890 | IEYSPL | 5 | 1235 | LNKDFK | 0.993 | Sequence | 0.799286 | 0.52 | <= |
| | 1303 | | NKDFKV | | | VED | | | | | SB |
| | | | EDK | | | | | | | | |
| 714 | DRB1_ | 891 | EYSPLN | 4 | 1235 | LNKDFK | 1 | Sequence | 0.693568 | 1.01 | <= |
| | 0301 | | KDFKVE | | | VED | | | | | WB |
| | | | DKD | | | | | | | | |
| 714 | DRB1_ | 891 | EYSPLN | 4 | 1235 | LNKDFK | 0.98 | Sequence | 0.358714 | 1.09 | <= |
| | 0305 | | KDFKVE | | | VED | | | | | WB |
| | | | DKD | | | | | | | | |
| 714 | DRB1_ | 891 | EYSPLN | 4 | 1235 | LNKDFK | 1 | Sequence | 0.891076 | 0.2 | <= |
| | 1303 | | KDFKVE | | | VED | | | | | SB |
| | | | DKD | | | | | | | | |
| 714 | DRB1_ | 891 | EYSPLN | 4 | 1235 | LNKDFK | 0.96 | Sequence | 0.285981 | 4.93 | <= |
| | 0803 | | KDFKVE | | | VED | | | | | WB |
| | | | DKD | | | | | | | | |
| 715 | DRB1_ | 892 | YSPLNK | 3 | 1235 | LNKDFK | 1 | Sequence | 0.792411 | 0.61 | <= |
| | 0301 | | DFKVED | | | VED | | | | | SB |
| | | | KDK | | | | | | | | |
| 715 | DRB1_ | 892 | YSPLNK | 3 | 1235 | LNKDFK | 1 | Sequence | 0.454134 | 0.68 | <= |
| | 0305 | | DFKVED | | | VED | | | | | SB |
| | | | KDK | | | | | | | | |
| 715 | DRB1_ | 892 | YSPLNK | 3 | 1235 | LNKDFK | 0.993 | Sequence | 0.506779 | 3.46 | <= |
| | 0801 | | DFKVED | | | VED | | | | | WB |
| | | | KDK | | | | | | | | |
| 715 | DRB1_ | 892 | YSPLNK | 3 | 1235 | LNKDFK | 0.993 | Sequence | 0.371527 | 3.47 | <= |
| | 1401 | | DFKVED | | | VED | | | | | WB |
| | | | KDK | | | | | | | | |
| 715 | DRB1_ | 892 | YSPLNK | 3 | 1235 | LNKDFK | 0.993 | Sequence | 0.371527 | 3.47 | <= |
| | 1454 | | DFKVED | | | VED | | | | | WB |
| | | | KDK | | | | | | | | |
| 715 | DRB1_ | 892 | YSPLNK | 3 | 1235 | LNKDFK | 0.993 | Sequence | 0.176247 | 3.97 | <= |
| | 1402 | | DFKVED | | | VED | | | | | WB |
| | | | KDK | | | | | | | | |
| 715 | DRB1_ | 892 | YSPLNK | 3 | 1235 | LNKDFK | 1 | Sequence | 0.923256 | 0.11 | <= |
| | 1303 | | DFKVED | | | VED | | | | | SB |
| | | | KDK | | | | | | | | |
| 715 | DRB1_ | 892 | YSPLNK | 3 | 1235 | LNKDFK | 0.967 | Sequence | 0.376277 | 2.64 | <= |
| | 0803 | | DFKVED | | | VED | | | | | WB |
| | | | KDK | | | | | | | | |
| 716 | DRB1_ | 893 | SPLNKD | 2 | 1235 | LNKDFK | 0.933 | Sequence | 0.677489 | 1.09 | <= |
| | 0301 | | FKVEDK | | | VED | | | | | WB |
| | | | DKK | | | | | | | | |
| 716 | DRB1_ | 893 | SPLNKD | 2 | 1235 | LNKDFK | 0.76 | Sequence | 0.373447 | 1.01 | <= |
| | 0305 | | FKVEDK | | | VED | | | | | WB |
| | | | DKK | | | | | | | | |
| 716 | DRB1_ | 893 | SPLNKD | 2 | 1235 | LNKDFK | 0.993 | Sequence | 0.858633 | 0.32 | <= |
| | 1303 | | FKVEDK | | | VED | | | | | SB |
| | | | DKK | | | | | | | | |
| 717 | DRB1_ | 894 | PLNKDF | 5 | 1236 | FKVEDK | 0.647 | Sequence | 0.518923 | 1.91 | <= |
| | 0301 | | KVEDKD | | | DKK | | | | | WB |
| | | | KKS | | | | | | | | |
| 717 | DRB1_ | 894 | PLNKDF | 5 | 1236 | FKVEDK | 0.76 | Sequence | 0.304305 | 1.42 | <= |
| | 0305 | | KVEDKD | | | DKK | | | | | WB |
| | | | KKS | | | | | | | | |
| 717 | DRB1_ | 894 | PLNKDF | 5 | 1236 | FKVEDK | 0.98 | Sequence | 0.360168 | 4.78 | <= |
| | 1101 | | KVEDKD | | | DKK | | | | | WB |
| | | | KKS | | | | | | | | |
| 717 | DRB1_ | 894 | PLNKDF | 1 | 1235 | LNKDFK | 0.673 | Sequence | 0.596066 | 1.57 | <= |
| | 1303 | | KVEDKD | | | VED | | | | | WB |
| | | | KKS | | | | | | | | |
| 718 | DRB1_ | 895 | LNKDFK | 4 | 1236 | FKVEDK | 0.993 | Sequence | 0.518852 | 1.91 | <= |
| | 0301 | | VEDKDK | | | DKK | | | | | WB |
| | | | KSE | | | | | | | | |
| 718 | DRB1_ | 895 | LNKDFK | 4 | 1236 | FKVEDK | 1 | Sequence | 0.356805 | 1.1 | <= |
| | 0305 | | VEDKDK | | | DKK | | | | | WB |
| | | | KSE | | | | | | | | |
| 718 | DRB1_ | 895 | LNKDFK | 4 | 1236 | FKVEDK | 1 | Sequence | 0.501368 | 3.05 | <= |
| | 1101 | | VEDKDK | | | DKK | | | | | WB |
| | | | KSE | | | | | | | | |
| 718 | DRB1_ | 895 | LNKDFK | 4 | 1236 | FKVEDK | 0.993 | Sequence | 0.41087 | 4.66 | <= |
| | 1104 | | VEDKDK | | | DKK | | | | | WB |
| | | | KSE | | | | | | | | |
| 718 | DRB1_ | 895 | LNKDFK | 4 | 1236 | FKVEDK | 0.98 | Sequence | 0.20704 | 2.87 | <= |
| | 1402 | | VEDKDK | | | DKK | | | | | WB |
| | | | KSE | | | | | | | | |
| 718 | DRB1_ | 895 | LNKDFK | 4 | 1236 | FKVEDK | 0.973 | Sequence | 0.411311 | 3.37 | <= |
| | 1303 | | VEDKDK | | | DKK | | | | | WB |
| | | | KSE | | | | | | | | |
| 719 | DRB1_ | 896 | NKDFKV | 3 | 1236 | FKVEDK | 0.993 | Sequence | 0.557692 | 1.7 | <= |
| | 0301 | | EDKDKK | | | DKK | | | | | WB |
| | | | SEH | | | | | | | | |
| 719 | DRB1_ | 896 | NKDFKV | 3 | 1236 | FKVEDK | 1 | Sequence | 0.388095 | 0.95 | <= |
| | 0305 | | EDKDKK | | | DKK | | | | | SB |
| | | | SEH | | | | | | | | |
| 719 | DRB1_ | 896 | NKDFKV | 3 | 1236 | FKVEDK | 0.993 | Sequence | 0.552377 | 2.59 | <= |
| | 1101 | | EDKDKK | | | DKK | | | | | WB |
| | | | SEH | | | | | | | | |
| 719 | DRB1_ | 896 | NKDFKV | 3 | 1236 | FKVEDK | 0.967 | Sequence | 0.473755 | 3.77 | <= |
| | 1104 | | EDKDKK | | | DKK | | | | | WB |
| | | | SEH | | | | | | | | |
| 719 | DRB1_ | 896 | NKDFKV | 3 | 1236 | FKVEDK | 0.987 | Sequence | 0.240955 | 1.97 | <= |
| | 1402 | | EDKDKK | | | DKK | | | | | WB |
| | | | SEH | | | | | | | | |
| 719 | DRB1_ | 896 | NKDFKV | 3 | 1236 | FKVEDK | 0.907 | Sequence | 0.461185 | 2.8 | <= |
| | 1303 | | EDKDKK | | | DKK | | | | | WB |
| | | | SEH | | | | | | | | |
| 719 | DRB1_ | 896 | NKDFKV | 3 | 1236 | FKVEDK | 0.88 | Sequence | 0.299469 | 4.91 | <= |
| | 1302 | | EDKDKK | | | DKK | | | | | WB |
| | | | SEH | | | | | | | | |
| 720 | DRB1_ | 897 | KDFKVE | 2 | 1236 | FKVEDK | 0.96 | Sequence | 0.397373 | 2.83 | <= |
| | 0301 | | DKDKKS | | | DKK | | | | | WB |
| | | | EHS | | | | | | | | |
| 720 | DRB1_ | 897 | KDFKVE | 2 | 1236 | FKVEDK | 0.973 | Sequenc | 0.244366 | 1.97 | <= |
| | 0305 | | DKDKKS | | | DKK | | | | | WB |
| | | | EHS | | | | | | | | |
| 720 | DRB1_ | 897 | KDFKVE | 2 | 1236 | FKVEDK | 0.687 | Sequence | 0.306578 | 4.95 | <= |
| | 1303 | | DKDKKS | | | DKK | | | | | WB |
| | | | EHS | | | | | | | | |
| 730 | DRB1_ | 898 | KSEHSI | 5 | 1237 | IINQIN | 0.993 | Sequence | 0.22634 | 4.44 | <= |
| | 1501 | | INQINL | | | LNL | | | | | WB |
| | | | NLL | | | | | | | | |
| 731 | DRB1_ | 899 | SEHSII | 4 | 1237 | IINQIN | 1 | Sequence | 0.426451 | 3.5 | <= |
| | 0404 | | NQINLN | | | LNL | | | | | WB |
| | | | LLP | | | | | | | | |
| 731 | DRB1_ | 899 | SEHSII | 4 | 1237 | IINQIN | 0.533 | Sequence | 0.361945 | 2.94 | <= |
| | 1201 | | NQINLN | | | LNL | | | | | WB |
| | | | LLP | | | | | | | | |
| 731 | DRB1_ | 899 | SEHSII | 1 | 1237 | IINQIN | 0.993 | Sequence | 0.510975 | 1.63 | <= |
| | 1501 | | NQINLN | | | LNL | | | | | WB |
| | | | LLP | | | | | | | | |
| 731 | DRB1_ | 899 | SEHSII | 4 | 1237 | IINQIN | 0.94 | Sequence | 0.287819 | 2.57 | <= |
| | 1503 | | NQINLN | | | LNL | | | | | WB |
| | | | LLP | | | | | | | | |
| 732 | DRB1_ | 900 | EHSIIN | 3 | 1237 | IINQIN | 0.973 | Sequence | 0.394314 | 3.94 | <= |
| | 0404 | | QINLNL | | | LNL | | | | | WB |
| | | | LPF | | | | | | | | |
| 732 | DRB1_ | 900 | EHSIIN | 4 | 1238 | INQINL | 0.487 | Sequence | 0.357975 | 3 | <= |
| | 1201 | | QINLNL | | | NLL | | | | | WB |
| | | | LPF | | | | | | | | |
| 732 | DRB1_ | 900 | EHSIIN | 3 | 1237 | IINQIN | 0.987 | Sequence | 0.479406 | 1.8 | <= |
| | 1501 | | QINLNL | | | LNL | | | | | WB |
| | | | LPF | | | | | | | | |
| 732 | DRB1_ | 900 | EHSIIN | 3 | 1237 | IINQIN | 0.92 | Sequence | 0.265528 | 2.87 | <= |
| | 1503 | | QINLNL | | | LNL | | | | | WB |
| | | | LPF | | | | | | | | |
| 735 | DRB1_ | 901 | IINQIN | 6 | 1239 | LNLLPF | 0.593 | Sequence | 0.273931 | 4.9 | <= |
| | 1201 | | LNLLPF | | | IRY | | | | | WB |
| | | | IRY | | | | | | | | |
| 736 | DRB1_ | 902 | INQINL | 5 | 1239 | LNLLPF | 0.807 | Sequence | 0.522216 | 1.2 | <= |
| | 1201 | | NLLPFI | | | IRY | | | | | WB |
| | | | RYP | | | | | | | | |
| 737 | DRB1_ | 903 | NQINLN | 4 | 1239 | LNLLPF | 0.827 | Sequence | 0.518964 | 1.22 | <= |
| | 1201 | | LLPFIR | | | IRY | | | | | WB |
| | | | YPL | | | | | | | | |
| 738 | DRB1_ | 904 | QINLNL | 3 | 1239 | LNLLPF | 0.74 | Sequence | 0.52223 | 1.2 | <= |
| | 1201 | | LPFIRY | | | IRY | | | | | WB |
| | | | PLF | | | | | | | | |
| 738 | DRB1_ | 904 | QINLNL | 5 | 1240 | LLPFIR | 0.993 | Sequence | 0.5573 | 2.84 | <= |
| | 1301 | | LPFIR | | | YPL | | | | | WB |
| | | | YPLF | | | | | | | | |
| 739 | DRB1_ | 905 | INLNLL | 2 | 1239 | LNLLPF | 0.533 | Sequence | 0.402995 | 2.34 | <= |
| | 1201 | | PFIRYP | | | IRY | | | | | WB |
| | | | LFS | | | | | | | | |
| 739 | DRB1_ | 905 | INLNLL | 4 | 1240 | LLPFIR | 1 | Sequence | 0.707457 | 1.25 | <= |
| | 1301 | | PFIRYP | | | YPL | | | | | WB |
| | | | LFS | | | | | | | | |
| 739 | DRB1_ | 905 | INLNLL | 4 | 1240 | LLPFIR | 0.987 | Sequence | 0.223437 | 3.59 | <= |
| | 1503 | | PFIRYP | | | YPL | | | | | WB |
| | | | LFS | | | | | | | | |
| 740 | DRB1_ | 906 | NLNLLP | 3 | 1240 | LLPFIR | 1 | Sequence | 0.452645 | 4.05 | <= |
| | 1104 | | FIRYPL | | | YPL | | | | | WB |
| | | | FSR | | | | | | | | |
| 740 | DRB1_ | 906 | NLNLLP | 3 | 1240 | LLPFIR | 0.727 | Sequence | 0.34137 | 3.3 | <= |
| | 1201 | | FIRYPL | | | YPL | | | | | WB |
| | | | FSR | | | | | | | | |
| 740 | DRB1_ | 906 | NLNLLP | 3 | 1240 | LLPFIR | 1 | Sequence | 0.802022 | 0.6 | <= |
| | 1301 | | FIRYPL | | | YPL | | | | | SB |
| | | | FSR | | | | | | | | |
| 740 | DRB1_ | 906 | NLNLLP | 3 | 1240 | LLPFIR | 0.98 | Sequence | 0.357632 | 3.75 | <= |
| | 1401 | | FIRYPL | | | YPL | | | | | WB |
| | | | FSR | | | | | | | | |
| 740 | DRB1_ | 906 | NLNLLP | 3 | 1240 | LLPFIR | 0.94 | Sequence | 0.234255 | 4.28 | <= |
| | 1501 | | FIRYPL | | | YPL | | | | | WB |
| | | | FSR | | | | | | | | |
| 740 | DRB1_ | 906 | NLNLLP | 3 | 1240 | LLPFIR | 0.967 | Sequence | 0.28774 | 2.57 | <= |
| | 1503 | | FIRYPL | | | YPL | | | | | WB |
| | | | FSR | | | | | | | | |
| 740 | DRB1_ | 906 | NLNLLP | 3 | 1240 | LLPFIR | 0.98 | Sequence | 0.357632 | 3.75 | <= |
| | 1454 | | FIRYPL | | | YPL | | | | | WB |
| | | | FSR | | | | | | | | |
| 740 | DRB1_ | 906 | NLNLLP | 3 | 1240 | LLPFIR | 0.973 | Sequence | 0.316964 | 4.32 | <= |
| | 1302 | | FIRYPL | | | YPL | | | | | WB |
| | | | FSR | | | | | | | | |
| 741 | DRB1_ | 907 | LNLLPF | 2 | 1240 | LLPFIR | 0.913 | Sequence | 0.582461 | 2.53 | <= |
| | 1301 | | IRYPLF | | | YPL | | | | | WB |
| | | | SRS | | | | | | | | |
| 741 | DRB1_ | 907 | LNLLPF | 2 | 1240 | LLPFIR | 0.7 | Sequence | 0.180932 | 4.64 | <= |
| | 1503 | | IRYPLF | | | YPL | | | | | WB |
| | | | SRS | | | | | | | | |
| 748 | DRB1_ | 806 | RYPLFS | 3 | 1241 | LFSRST | 0.987 | Sequence | 0.535989 | 3.12 | <= |
| | 1301 | | RSTLKF | | | LKF | | | | | WB |
| | | | ENK | | | | | | | | |
| 751 | DRB1_ | 909 | LFSRST | 6 | 1242 | LKFENK | 1 | Sequence | 0.346852 | 3.97 | <= |
| | 1401 | | LKFENK | | | ATL | | | | | WB |
| | | | ATL | | | | | | | | |
| 751 | DRB1_ | 909 | LFSRST | 6 | 1242 | LKFENK | 1 | Sequence | 0.346852 | 3.97 | <= |
| | 1454 | | LKFENK | | | ATL | | | | | WB |
| | | | ATL | | | | | | | | |
| 752 | DRB1_ | 910 | FSRSTL | 5 | 1242 | LKFENK | 1 | Sequence | 0.299953 | 3.79 | <= |
| | 0301 | | KFENKA | | | ATL | | | | | WB |
| | | | TLY | | | | | | | | |
| 752 | DRB1_ | 910 | FSRSTL | 5 | 1242 | LKFENK | 1 | Sequence | 0.440906 | 4.72 | <= |
| | 1301 | | KFENKA | | | ATL | | | | | WB |
| | | | TLY | | | | | | | | |
| 752 | DRB1_ | 910 | FSRSTL | 5 | 1242 | LKFENK | 1 | Sequence | 0.589969 | 1.02 | <= |
| | 1401 | | KFENKA | | | ATL | | | | | WB |
| | | | TLY | | | | | | | | |
| 752 | DRB1_ | 910 | FSRSTL | 5 | 1242 | LKFENK | 1 | Sequence | 0.589969 | 1.02 | <= |
| | 1454 | | KFENKA | | | ATL | | | | | WB |
| | | | TLY | | | | | | | | |
| 752 | DRB1_ | 910 | FSRSTL | 5 | 1242 | LKFENK | 0.987 | Sequence | 0.168525 | 4.31 | <= |
| | 1402 | | KFENKA | | | ATL | | | | | WB |
| | | | TLY | | | | | | | | |
| 752 | DRB1_ | 910 | FSRSTL | 5 | 1242 | LKFENK | 0.967 | Sequence | 0.311366 | 4.48 | <= |
| | 1302 | | KFENKA | | | ATL | | | | | WB |
| | | | TLY | | | | | | | | |
| 753 | DRB1_ | 911 | SRSTLK | 4 | 1242 | LKFENK | 1 | Sequence | 0.429307 | 2.55 | <= |
| | 0301 | | FENKAT | | | ATL | | | | | WB |
| | | | LYS | | | | | | | | |
| 753 | DRB1_ | 911 | SRSTLK | 4 | 1242 | LKFENK | 0.96 | Sequence | 0.106748 | 4.55 | <= |
| | 0305 | | FENKAT | | | ATL | | | | | WB |
| | | | LYS | | | | | | | | |
| 753 | DRB1_ | 911 | SRSTLK | 1 | 1242 | LKFENK | 0.987 | Sequence | 0.4672 | 3.85 | <= |
| | 1104 | | FENKAT | | | ATL | | | | | WB |
| | | | LYS | | | | | | | | |
| 753 | DRB1_ | 911 | SRSTLK | 4 | 1242 | LKFENK | 1 | Sequence | 0.597198 | 2.37 | <= |
| | 1301 | | FENKAT | | | ATL | | | | | WB |
| | | | LYS | | | | | | | | |
| 753 | DRB1_ | 911 | SRSTLK | 4 | 1242 | LKFENK | 1 | Sequence | 0.657477 | 0.65 | <= |
| | 1401 | | FENKAT | | | ATL | | | | | SB |
| | | | LYS | | | | | | | | |
| 753 | DRB1_ | 911 | SRSTLK | 4 | 1242 | LKFENK | 1 | Sequence | 0.657477 | 0.65 | <= |
| | 1454 | | FENKAT | | | ATL | | | | | SB |
| | | | LYS | | | | | | | | |
| 753 | DRB1_ | 911 | SRSTLK | 4 | 1242 | LKFENK | 0.953 | Sequence | 0.243681 | 1.92 | <= |
| | 1402 | | FENKAT | | | ATL | | | | | WB |
| | | | LYS | | | | | | | | |
| 753 | DRB1_ | 911 | SRSTLK | 4 | 1242 | LKFENK | 0.94 | Sequence | 0.423488 | 1.89 | <= |
| | 1302 | | FENKAT | | | ATL | | | | | WB |
| | | | LYS | | | | | | | | |
| 754 | DRB1_ | 912 | RSTLKF | 3 | 1242 | LKFENK | 1 | Sequence | 0.422762 | 2.61 | <= |
| | 0301 | | ENKATL | | | ATL | | | | | WB |
| | | | YSF | | | | | | | | |
| 754 | DRB1_ | 912 | RSTLKF | 3 | 1242 | LKFENK | 0.92 | Sequence | 0.106322 | 4.57 | <= |
| | 0305 | | ENKATL | | | ATL | | | | | WB |
| | | | YSF | | | | | | | | |
| 754 | DRB1_ | 912 | RSTLKF | 5 | 1243 | FENKAT | 0.727 | Sequence | 0.509778 | 3.41 | <= |
| | 0801 | | ENKATL | | | LYS | | | | | WB |
| | | | YSF | | | | | | | | |
| 754 | DRB1_ | 912 | RSTLKF | 3 | 1242 | LKFENK | 0.513 | Sequence | 0.349309 | 4.96 | <= |
| | 1101 | | ENKATL | | | ATL | | | | | WB |
| | | | YSF | | | | | | | | |
| 754 | DRB1_ | 912 | RSTLKF | 3 | 1242 | LKFENK | 0.9 | Sequence | 0.45079 | 4.07 | <= |
| | 1104 | | ENKATL | | | ATL | | | | | WB |
| | | | YSF | | | | | | | | |
| 754 | DRB1_ | 912 | RSTLKF | 3 | 1242 | LKFENK | 0.973 | Sequence | 0.580282 | 2.56 | <= |
| | 1301 | | ENKATL | | | ATL | | | | | WB |
| | | | YSF | | | | | | | | |
| 754 | DRB1_ | 912 | RSTLKF | 3 | 1242 | LKFENK | 1 | Sequence | 0.631858 | 0.78 | <= |
| | 1401 | | ENKATL | | | ATL | | | | | SB |
| | | | YSF | | | | | | | | |
| 754 | DRB1_ | 912 | RSTLKF | 3 | 1242 | LKFENK | 1 | Sequence | 0.631858 | 0.78 | <= |
| | 1454 | | ENKATL | | | ATL | | | | | SB |
| | | | YSF | | | | | | | | |
| 754 | DRB1_ | 912 | RSTLKF | 3 | 1242 | LKFENK | 0.887 | Sequence | 0.245296 | 1.88 | <= |
| | 1402 | | ENKATL | | | ATL | | | | | WB |
| | | | YSF | | | | | | | | |
| 754 | DRB1_ | 912 | RSTLKF | 3 | 1242 | LKFENK | 0.887 | Sequence | 0.421483 | 1.92 | <= |
| | 1302 | | ENKATL | | | ATL | | | | | WB |
| | | | YSF | | | | | | | | |
| 754 | DRB1_ | 912 | RSTLKF | 5 | 1243 | FENKAT | 0.647 | Sequence | 0.34102 | 3.36 | <= |
| | 0803 | | ENKATL | | | LYS | | | | | WB |
| | | | YSF | | | | | | | | |
| 755 | DRB1_ | 913 | STLKFE | 4 | 1243 | FENKAT | 0.993 | Sequence | 0.350449 | 3.11 | <= |
| | 0402 | | NKATLY | | | LYS | | | | | WB |
| | | | SFN | | | | | | | | |
| 755 | DRB1_ | 913 | STLKFE | 4 | 1243 | FENKAT | 0.927 | Sequence | 0.576974 | 2.49 | <= |
| | 0801 | | NKATLY | | | LYS | | | | | WB |
| | | | SFN | | | | | | | | |
| 755 | DRB1_ | 913 | STLKFE | 4 | 1243 | FENKAT | 0.92 | Sequence | 0.394273 | 4.3 | <= |
| | 1101 | | NKATLY | | | LYS | | | | | WB |
| | | | SFN | | | | | | | | |
| 755 | DRB1_ | 913 | STLKFE | 2 | 1242 | LKFENK | 0.96 | Sequence | 0.391122 | 3.1 | <= |
| | 1401 | | NKATLY | | | ATL | | | | | WB |
| | | | SFN | | | | | | | | |
| 755 | DRB1_ | 913 | STLKFE | 2 | 1242 | LKFENK | 0.96 | Sequence | 0.391122 | 3.1 | <= |
| | 1454 | | NKATLY | | | ATL | | | | | WB |
| | | | SFN | | | | | | | | |
| 755 | DRB1_ | 913 | STLKFE | 4 | 1243 | FENKAT | 0.847 | Sequence | 0.387681 | 2.45 | <= |
| | 0803 | | NKATLY | | | LYS | | | | | WB |
| | | | SFN | | | | | | | | |
| 756 | DRB1_ | 914 | TLKFEN | 3 | 1243 | FENKAT | 0.993 | Sequence | 0.331028 | 3.31 | <= |
| | 0103 | | KATLYS | | | LYS | | | | | WB |
| | | | FNK | | | | | | | | |
| 756 | DRB1_ | 914 | TLKFEN | 3 | 1243 | FENKAT | 1 | Sequence | 0.357669 | 3.58 | <= |
| | 0401 | | KATLYS | | | LYS | | | | | WB |
| | | | FNK | | | | | | | | |
| 756 | DRB1_ | 914 | TLKFEN | 3 | 1243 | FENKAT | 1 | Sequence | 0.475044 | 1.75 | <= |
| | 0402 | | KATLYS | | | LYS | | | | | WB |
| | | | FNK | | | | | | | | |
| 756 | DRB1_ | 914 | TLKFEN | 3 | 1243 | FENKAT | 0.993 | Sequence | 0.658713 | 1.64 | <= |
| | 0801 | | KATLYS | | | LYS | | | | | WB |
| | | | FNK | | | | | | | | |
| 756 | DRB1_ | 914 | TLKFEN | 3 | 1243 | FENKAT | 1 | Sequence | 0.538931 | 2.71 | <= |
| | 1101 | | KATLYS | | | LYS | | | | | WB |
| | | | FNK | | | | | | | | |
| 756 | DRB1_ | 914 | TLKFEN | 3 | 1243 | FENKAT | 0.98 | Sequence | 0.452766 | 1.54 | <= |
| | 0803 | | KATLYS | | | LYS | | | | | WB |
| | | | FNK | | | | | | | | |
| 756 | DRB1_ | 914 | TLKFEN | 3 | 1243 | FENKAT | 1 | Sequence | 0.345863 | 4.53 | <= |
| | 0408 | | KATLYS | | | LYS | | | | | WB |
| | | | FNK | | | | | | | | |
| 757 | DRB1_ | 915 | LKFENK | 2 | 1243 | FENKAT | 1 | Sequence | 0.269586 | 4.59 | <= |
| | 0402 | | ATLYSF | | | LYS | | | | | WB |
| | | | NKK | | | | | | | | |
| 757 | DRB1_ | 915 | LKFENK | 2 | 1243 | FENKAT | 0.993 | Sequence | 0.460981 | 4.24 | <= |
| | 0801 | | ATLYSF | | | LYS | | | | | WB |
| | | | NKK | | | | | | | | |
| 760 | DRB1_ | 916 | ENKATL | 5 | 1244 | LYSFNK | 0.927 | Sequence | 0.625575 | 1.96 | <= |
| | 0801 | | YSFNKK | | | KYD | | | | | WB |
| | | | YDS | | | | | | | | |
| 760 | DRB1_ | 916 | ENKATL | 5 | 1244 | LYSFNK | 0.853 | Sequence | 0.409786 | 3.39 | <= |
| | 1303 | | YSFNKK | | | KYD | | | | | WB |
| | | | YDS | | | | | | | | |
| 760 | DRB1_ | 916 | ENKATL | 5 | 1244 | LYSFNK | 0.9 | Sequence | 0.406957 | 2.14 | <= |
| | 0803 | | YSFNKK | | | KYD | | | | | WB |
| | | | YDS | | | | | | | | |
| 761 | DRB1_ | 917 | NKATLY | 4 | 1244 | LYSFNK | 0.847 | Sequence | 0.793869 | 0.65 | <= |
| | 0801 | | SFNKKY | | | KYD | | | | | SB |
| | | | DSD | | | | | | | | |
| 761 | DRB1_ | 917 | NKATLY | 5 | 1245 | YSFNKK | 0.733 | Sequence | 0.642574 | 1.88 | <= |
| | 1101 | | SFNKKY | | | YDS | | | | | WB |
| | | | DSD | | | | | | | | |
| 761 | DRB1_ | 917 | NKATLY | 4 | 1244 | LYSFNK | 0.773 | Sequence | 0.553507 | 2.84 | <= |
| | 1104 | | SFNKKY | | | KYD | | | | | WB |
| | | | DSD | | | | | | | | |
| 761 | DRB1_ | 917 | NKATLY | 4 | 1244 | LYSFNK | 0.867 | Sequence | 0.598405 | 2.35 | <= |
| | 1301 | | SFNKKY | | | KYD | | | | | WB |
| | | | DSD | | | | | | | | |
| 761 | DRB1_ | 917 | NKATLY | 4 | 1244 | LYSFNK | 0.667 | Sequence | 0.661975 | 1.17 | <= |
| | 1303 | | SFNKKY | | | KYD | | | | | WB |
| | | | DSD | | | | | | | | |
| 761 | DRB1_ | 917 | NKATLY | 5 | 1245 | YSFNKK | 0.58 | Sequence | 0.456085 | 1.5 | <= |
| | 1302 | | SFNKKY | | | YDS | | | | | WB |
| | | | DSD | | | | | | | | |
| 761 | DRB1_ | 917 | NKATLY | 4 | 1244 | LYSFNK | 0.853 | Sequence | 0.562261 | 0.7 | <= |
| | 0803 | | SFNKKY | | | KYD | | | | | SB |
| | | | DSD | | | | | | | | |
| 762 | DRB1_ | 918 | KATLYS | 3 | 1244 | LYSFNK | 0.8 | Sequence | 0.802747 | 0.61 | <= |
| | 0801 | | FNKKYD | | | KYD | | | | | SB |
| | | | SDV | | | | | | | | |
| 762 | DRB1_ | 918 | KATLYS | 4 | 1245 | YSFNKK | 0.867 | Sequence | 0.739589 | 1.3 | <= |
| | 1101 | | FNKKYD | | | YDS | | | | | WB |
| | | | SDV | | | | | | | | |
| 762 | DRB1_ | 918 | KATLYS | 3 | 1244 | LYSFNK | 0.707 | Sequence | 0.600944 | 2.36 | <= |
| | 1104 | | FNKKYD | | | KYD | | | | | WB |
| | | | SDV | | | | | | | | |
| 762 | DRB1_ | 918 | KATLYS | 3 | 1244 | LYSFNK | 0.867 | Sequence | 0.62981 | 2.02 | <= |
| | 1301 | | FNKKYD | | | KYD | | | | | WB |
| | | | SDV | | | | | | | | |
| 762 | DRB1_ | 918 | KATLYS | 3 | 1244 | LYSFNK | 0.593 | Sequence | 0.305296 | 4.99 | <= |
| | 1401 | | FNKKYD | | | KYD | | | | | WB |
| | | | SDV | | | | | | | | |
| 762 | DRB1_ | 918 | KATLYS | 3 | 1244 | LYSFNK | 0.593 | Sequence | 0.305296 | 4.99 | <= |
| | 1454 | | FNKKYD | | | KYD | | | | | WB |
| | | | SDV | | | | | | | | |
| 762 | DRB1_ | 918 | KATLYS | 4 | 1245 | YSFNKK | 0.8 | Sequence | 0.183464 | 3.69 | <= |
| | 1402 | | FNKKYD | | | YDS | | | | | WB |
| | | | SDV | | | | | | | | |
| 762 | DRB1_ | 918 | KATLYS | 3 | 1244 | LYSFNK | 0.6 | Sequence | 0.693743 | 1.01 | <= |
| | 1303 | | FNKKYD | | | KYD | | | | | WB |
| | | | SDV | | | | | | | | |
| 762 | DRB1_ | 918 | KATLYS | 4 | 1245 | YSFNKK | 0.647 | Sequence | 0.524477 | 0.81 | <= |
| | 1302 | | FNKKYD | | | YDS | | | | | SB |
| | | | SDV | | | | | | | | |
| 762 | DRB1_ | 918 | KATLYS | 3 | 1244 | LYSFNK | 0.84 | Sequence | 0.5743 | 0.63 | <= |
| | 0803 | | FNKKYD | | | KYD | | | | | SB |
| | | | SDV | | | | | | | | |
| 763 | DRB1_ | 919 | ATLYSF | 3 | 1245 | YSFNKK | 0.553 | Sequence | 0.702614 | 1.28 | <= |
| | 0801 | | NKKYDS | | | YDS | | | | | WB |
| | | | DVK | | | | | | | | |
| 763 | DRB1_ | 919 | ATLYSF | 3 | 1245 | YSFNKK | 0.987 | Sequence | 0.815981 | 0.87 | <= |
| | 1101 | | NKKYDS | | | YDS | | | | | SB |
| | | | DVK | | | | | | | | |
| 763 | DRB1_ | 919 | ATLYSF | 3 | 1245 | YSFNKK | 0.7 | Sequence | 0.509041 | 3.33 | <= |
| | 1104 | | NKKYDS | | | YDS | | | | | WB |
| | | | DVK | | | | | | | | |
| 763 | DRB1_ | 919 | ATLYSF | 2 | 1244 | LYSFNK | 0.52 | Sequence | 0.468923 | 4.21 | <= |
| | 1301 | | NKKYDS | | | KYD | | | | | WB |
| | | | DVK | | | | | | | | |
| 763 | DRB1_ | 919 | ATLYSF | 3 | 1245 | YSFNKK | 0.96 | Sequence | 0.228181 | 2.29 | <= |
| | 1402 | | NKKYDS | | | YDS | | | | | WB |
| | | | DVK | | | | | | | | |
| 763 | DRB1_ | 919 | ATLYSF | 3 | 1245 | YSFNKK | 0.78 | Sequence | 0.620306 | 1.41 | <= |
| | 1303 | | NKKYDS | | | YDS | | | | | WB |
| | | | DVK | | | | | | | | |
| 763 | DRB1_ | 919 | ATLYSF | 3 | 1245 | YSFNKK | 0.867 | Sequence | 0.545933 | 0.67 | <= |
| | 1302 | | NKKYDS | | | YDS | | | | | SB |
| | | | DVK | | | | | | | | |
| 763 | DRB1_ | 919 | ATLYSF | 2 | 1244 | LYSFNK | 0.52 | Sequence | 0.437954 | 1.71 | <= |
| | 0803 | | NKKYDS | | | KYD | | | | | WB |
| | | | DVK | | | | | | | | |
| 764 | DRB1_ | 920 | TLYSFN | 2 | 1245 | YSFNKK | 0.993 | Sequence | 0.574242 | 2.41 | <= |
| | 1101 | | KKYDSD | | | YDS | | | | | WB |
| | | | VKS | | | | | | | | |
| 764 | DRB1_ | 920 | TLYSFN | 2 | 1245 | YSFNKK | 0.92 | Sequence | 0.339499 | 4.37 | <= |
| | 1303 | | KKYDSD | | | YDS | | | | | WB |
| | | | VKS | | | | | | | | |
| 764 | DRB1_ | 920 | TLYSFN | 2 | 1245 | YSFNKK | 0.893 | Sequence | 0.344172 | 3.5 | <= |
| | 1302 | | KKYDSD | | | YDS | | | | | WB |
| | | | VKS | | | | | | | | |
| 766 | DRB1_ | 921 | YSFNKK | 6 | 1246 | YDSDVK | 0.94 | Sequence | 0.244395 | 4.61 | <= |
| | 0301 | | YDSDVK | | | SLV | | | | | WB |
| | | | SLV | | | | | | | | |
| 766 | DRB1_ | 921 | YSFNKK | 6 | 1246 | YDSDVK | 0.987 | Sequence | 0.434034 | 0.76 | <= |
| | 0305 | | YDSDVK | | | SLV | | | | | SB |
| | | | SLV | | | | | | | | |
| 767 | DRB1_ | 922 | SFNKKY | 5 | 1246 | YDSDVK | 1 | Sequence | 0.528212 | 1.85 | <= |
| | 0301 | | DSDVKS | | | SLV | | | | | WB |
| | | | LVN | | | | | | | | |
| 767 | DRB1_ | 922 | SFNKKY | 5 | 1246 | YDSDVK | 1 | Sequence | 0.736689 | 0.09 | <= |
| | 0305 | | DSDVKS | | | SLV | | | | | SB |
| | | | LVN | | | | | | | | |
| 767 | DRB1_ | 922 | SFNKKY | 5 | 1246 | YDSDVK | 0.96 | Sequence | 0.307574 | 1.04 | <= |
| | 1402 | | DSDVKS | | | SLV | | | | | WB |
| | | | LVN | | | | | | | | |
| 767 | DRB303 | 922 | SFNKKY | 5 | 1246 | YDSDVK | 1 | Sequence | 0.621902 | 1.4 | <= |
| | | | DSDVKS | | | SLV | | | | | WB |
| | | | LVN | | | | | | | | |
| 768 | DRB1_ | 923 | FNKKYD | 4 | 1246 | YDSDVK | 1 | Sequence | 0.66176 | 1.17 | <= |
| | 0301 | | SDVKSL | | | SLV | | | | | WB |
| | | | VNK | | | | | | | | |
| 768 | DRB1_ | 923 | FNKKYD | 4 | 1246 | YDSDVK | 1 | Sequence | 0.823611 | 0.02 | <= |
| | 0305 | | SDVKSL | | | SLV | | | | | SB |
| | | | VNK | | | | | | | | |
| 768 | DRB1_ | 923 | FNKKYD | 4 | 1246 | YDSDVK | 1 | Sequence | 0.354007 | 4.88 | <= |
| | 1101 | | SDVKSL | | | SLV | | | | | WB |
| | | | VNK | | | | | | | | |
| 768 | DRB1_ | 923 | FNKKYD | 4 | 1246 | YDSDVK | 0.993 | Sequence | 0.329383 | 4.39 | <= |
| | 1401 | | SDVKSL | | | SLV | | | | | WB |
| | | | VNK | | | | | | | | |
| 768 | DRB1_ | 923 | FNKKYD | 4 | 1246 | YDSDVK | 0.993 | Sequence | 0.329383 | 4.39 | <= |
| | 1454 | | SDVKSL | | | SLV | | | | | WB |
| | | | VNK | | | | | | | | |
| 768 | DRB1_ | 923 | FNKKYD | 4 | 1246 | YDSDVK | 1 | Sequence | 0.451666 | 0.24 | <= |
| | 1402 | | SDVKSL | | | SLV | | | | | SB |
| | | | VNK | | | | | | | | |
| 768 | DRB1_ | 923 | FNKKYD | 4 | 1246 | YDSDVK | 1 | Sequence | 0.752791 | 0.71 | <= |
| | 1303 | | SDVKSL | | | SLV | | | | | SB |
| | | | VNK | | | | | | | | |
| 768 | DRB1_ | 923 | FNKKYD | 4 | 1246 | YDSDVK | 0.973 | Sequence | 0.367057 | 2.92 | <= |
| | 1302 | | SDVKSL | | | SLV | | | | | WB |
| | | | VNK | | | | | | | | |
| 768 | DRB1_ | 923 | FNKKYD | 4 | 1246 | YDSDVK | 0.967 | Sequence | 0.360492 | 2.93 | <= |
| | 0803 | | SDVKSL | | | SLV | | | | | WB |
| | | | VNK | | | | | | | | |
| 769 | DRB1_ | 924 | NKKYDS | 3 | 1246 | YDSDVK | 1 | Sequence | 0.706238 | 0.96 | <= |
| | 0301 | | DVKSLV | | | SLV | | | | | SB |
| | | | NKN | | | | | | | | |
| 769 | DRB1_ | 924 | NKKYDS | 3 | 1246 | YDSDVK | 1 | Sequence | 0.845492 | 0.01 | <= |
| | 0305 | | DVKSLV | | | SLV | | | | | SB |
| | | | NKN | | | | | | | | |
| 769 | DRB1_ | 924 | NKKYDS | 3 | 1246 | YDSDVK | 0.993 | Sequence | 0.463767 | 4.18 | <= |
| | 0801 | | DVKSLV | | | SLV | | | | | WB |
| | | | NKN | | | | | | | | |
| 769 | DRB1_ | 924 | NKKYDS | 3 | 1246 | YDSDVK | 1 | Sequence | 0.433517 | 3.83 | <= |
| | 1101 | | DVKSLV | | | SLV | | | | | WB |
| | | | NKN | | | | | | | | |
| 769 | DRB1_ | 924 | NKKYDS | 3 | 1246 | YDSDVK | 0.993 | Sequence | 0.361589 | 3.67 | <= |
| | 1401 | | DVKSLV | | | SLV | | | | | WB |
| | | | NKN | | | | | | | | |
| 769 | DRB1_ | 924 | NKKYDS | 3 | 1246 | YDSDVK | 0.993 | Sequence | 0.361589 | 3.67 | <= |
| | 1454 | | DVKSLV | | | SLV | | | | | WB |
| | | | NKN | | | | | | | | |
| 769 | DRB1_ | 924 | NKKYDS | 3 | 1246 | YDSDVK | 1 | Sequence | 0.503208 | 0.13 | <= |
| | 1402 | | DVKSLV | | | SLV | | | | | SB |
| | | | NKN | | | | | | | | |
| 769 | DRB1_ | 924 | NKKYDS | 3 | 1246 | YDSDVK | 1 | Sequence | 0.785324 | 0.58 | <= |
| | 1303 | | DVKSLV | | | SLV | | | | | SB |
| | | | NKN | | | | | | | | |
| 769 | DRB1_ | 924 | NKKYDS | 3 | 1246 | YDSDVK | 1 | Sequence | 0.426569 | 1.86 | <= |
| | 1302 | | DVKSLV | | | SLV | | | | | WB |
| | | | NKN | | | | | | | | |
| 769 | DRB1_ | 924 | NKKYDS | 3 | 1246 | YDSDVK | 0.98 | Sequence | 0.403884 | 2.19 | <= |
| | 0803 | | DVKSLV | | | SLV | | | | | WB |
| | | | NKN | | | | | | | | |
| 770 | DRB1_ | 925 | KKYDSD | 2 | 1246 | YDSDVK | 1 | Sequence | 0.52979 | 1.84 | <= |
| | 0301 | | VKSLVN | | | SLV | | | | | WB |
| | | | KNS | | | | | | | | |
| 770 | DRB1_ | 925 | KKYDSD | 2 | 1246 | YDSDVK | 1 | Sequence | 0.730545 | 0.09 | <= |
| | 0305 | | VKSLVN | | | SLV | | | | | SB |
| | | | KNS | | | | | | | | |
| 770 | DRB1_ | 925 | KKYDSD | 2 | 1246 | YDSDVK | 0.987 | Sequence | 0.318741 | 0.92 | <= |
| | 1402 | | VKSLVN | | | SLV | | | | | SB |
| | | | KNS | | | | | | | | |
| 770 | DRB1_ | 925 | KKYDSD | 2 | 1246 | YDSDVK | 1 | Sequence | 0.588975 | 1.62 | <= |
| | 1303 | | VKSLVN | | | SLV | | | | | WB |
| | | | KNS | | | | | | | | |
| 771 | DRB1_ | 926 | KYDSDV | 1 | 1246 | YDSDVK | 0.993 | Sequence | 0.359424 | 1.09 | <= |
| | 0305 | | KSLVNK | | | SLV | | | | | WB |
| | | | NSS | | | | | | | | |
| 771 | DRB1_ | 926 | KYDSDV | 5 | 1247 | VKSLVN | 1 | Sequence | 0.492664 | 1.62 | <= |
| | 0402 | | KSLVNK | | | KNS | | | | | WB |
| | | | NSS | | | | | | | | |
| 771 | DRB1_ | 926 | KYDSDV | 5 | 1247 | VKSLVN | 0.993 | Sequence | 0.519349 | 2.5 | <= |
| | 0404 | | KSLVNK | | | KNS | | | | | WB |
| | | | NSS | | | | | | | | |
| 772 | DRB1_ | 927 | YDSDVK | 4 | 1247 | VKSLVN | 1 | Sequence | 0.364724 | 4.69 | <= |
| | 0102 | | SLVNKN | | | KNS | | | | | WB |
| | | | SSI | | | | | | | | |
| 772 | DRB1_ | 927 | YDSDVK | 4 | 1247 | VKSLVN | 1 | Sequence | 0.592535 | 1 | <= |
| | 0402 | | SLVNKN | | | KNS | | | | | SB |
| | | | SSI | | | | | | | | |
| 772 | DRB1_ | 927 | YDSDVK | 4 | 1247 | VKSLVN | 1 | Sequence | 0.61902 | 1.72 | <= |
| | 0404 | | SLVNKN | | | KNS | | | | | WB |
| | | | SSI | | | | | | | | |
| 773 | DRB1_ | 928 | DSDVKS | 3 | 1247 | VKSLVN | 1 | Sequence | 0.390016 | 4.32 | <= |
| | 0102 | | LVNKNS | | | KNS | | | | | WB |
| | | | SIF | | | | | | | | |
| 773 | DRB1_ | 928 | DSDVKS | 3 | 1247 | VKSLVN | 1 | Sequence | 0.601032 | 0.96 | <= |
| | 0402 | | LVNKNS | | | KNS | | | | | SB |
| | | | SIF | | | | | | | | |
| 773 | DRB1_ | 928 | DSDVKS | 3 | 1247 | VKSLVN | 1 | Sequence | 0.621516 | 1.71 | <= |
| | 0404 | | LVNKNS | | | KNS | | | | | WB |
| | | | SIF | | | | | | | | |
| 774 | DRB1_ | 929 | SDVKSL | 2 | 1247 | VKSLVN | 0.967 | Sequence | 0.316864 | 3.64 | <= |
| | 0402 | | VNKNSS | | | KNS | | | | | WB |
| | | | IFL | | | | | | | | |
| 775 | DRB1_ | 930 | DVKSLV | 5 | 1248 | VNKNSS | 0.42 | Sequence | 0.334486 | 3.79 | <= |
| | 1302 | | NKNSSI | | | IFL | | | | | WB |
| | | | FLS | | | | | | | | |
| 776 | DRB1_ | 931 | VKSLVN | 4 | 1248 | VNKNSS | 0.527 | Sequence | 0.453927 | 1.52 | <= |
| | 1302 | | KNSSIF | | | IFL | | | | | WB |
| | | | LSD | | | | | | | | |
| 777 | DRB1_ | 932 | KSLVNK | 3 | 1248 | VNKNSS | 0.827 | Sequence | 0.451375 | 1.55 | <= |
| | 1302 | | NSSIFL | | | IFL | | | | | WB |
| | | | SDP | | | | | | | | |
| 781 | DRB1_ | 933 | NKNSSI | 6 | 1249 | FLSDPE | 0.807 | Sequence | 0.119032 | 4.13 | <= |
| | 0305 | | FLSDPE | | | TFY | | | | | WB |
| | | | TFY | | | | | | | | |
| 782 | DRB1_ | 934 | KNSSIF | 5 | 1249 | FLSDPE | 0.88 | Sequence | 0.440124 | 2.47 | <= |
| | 0301 | | LSDPET | | | TFY | | | | | WB |
| | | | FYQ | | | | | | | | |
| 782 | DRB1_ | 934 | KNSSIF | 5 | 1249 | FLSDPE | 0.953 | Sequence | 0.419766 | 0.81 | <= |
| | 0305 | | LSDPET | | | TFY | | | | | SB |
| | | | FYQ | | | | | | | | |
| 783 | DRB1_ | 935 | NSSIFL | 4 | 1249 | FLSDPE | 0.927 | Sequence | 0.578196 | 1.58 | <= |
| | 0301 | | SDPETF | | | TFY | | | | | WB |
| | | | YQS | | | | | | | | |
| 783 | DRB1_ | 935 | NSSIFL | 4 | 1249 | FLSDPE | 0.973 | Sequence | 0.550478 | 0.38 | <= |
| | 0305 | | SDPETF | | | TFY | | | | | SB |
| | | | YQS | | | | | | | | |
| 784 | DRB1_ | 936 | SSIFLS | 3 | 1249 | FLSDPE | 0.973 | Sequence | 0.510163 | 1.97 | <= |
| | 0301 | | DPETF | | | TFY | | | | | WB |
| | | | YQSL | | | | | | | | |
| 784 | DRB1_ | 936 | SSIFLS | 3 | 1249 | FLSDPE | 1 | Sequence | 0.4951 | 0.54 | <= |
| | 0305 | | DPETFY | | | TFY | | | | | SB |
| | | | QSL | | | | | | | | |
| 785 | DRB1_ | 937 | SIFLSD | 2 | 1249 | FLSDPE | 0.987 | Sequence | 0.282807 | 4.02 | <= |
| | 0301 | | PETFYQ | | | TFY | | | | | WB |
| | | | SLT | | | | | | | | |
| 785 | DRB1_ | 937 | SIFLSD | 2 | 1249 | FLSDPE | 1 | Sequence | 0.267637 | 1.73 | <= |
| | 0305 | | PETFYQ | | | TFY | | | | | WB |
| | | | SLT | | | | | | | | |
| 788 | DRB1_ | 938 | LSDPET | 6 | 1250 | FYQSLT | 0.993 | Sequence | 0.387329 | 2.73 | <= |
| | 0701 | | FYQSLT | | | ASL | | | | | WB |
| | | | ASL | | | | | | | | |
| 788 | DRB1_ | 938 | LSDPET | 6 | 1250 | FYQSLT | 0.98 | Sequence | 0.4745 | 2.58 | <= |
| | 0901 | | FYQSLT | | | ASL | | | | | WB |
| | | | ASL | | | | | | | | |
| 789 | DRB1_ | 939 | SDPETF | 6 | 1251 | YQSLTA | 0.92 | Sequence | 0.380779 | 3.59 | <= |
| | 0101 | | YQSLTA | | | SLI | | | | | WB |
| | | | SLI | | | | | | | | |
| 789 | DRB1_ | 939 | SDPETF | 5 | 1250 | FYQSLT | 0.953 | Sequence | 0.673282 | 0.82 | <= |
| | 0701 | | YQSLTA | | | ASL | | | | | SB |
| | | | SLI | | | | | | | | |
| 789 | DRB1_ | 939 | SDPETF | 5 | 1250 | FYQSLT | 0.767 | Sequence | 0.72657 | 0.49 | <= |
| | 0901 | | YQSLTA | | | ASL | | | | | SB |
| | | | SLI | | | | | | | | |
| 790 | DRB1_ | 940 | DPETFY | 5 | 1251 | YQSLTA | 0.993 | Sequence | 0.859449 | 0.59 | <= |
| | 0101 | | QSLTAS | | | SLI | | | | | SB |
| | | | LIY | | | | | | | | |
| 790 | DRB1_ | 940 | DPETFY | 5 | 1251 | YQSLTA | 0.953 | Sequence | 0.548087 | 2.6 | <= |
| | 0102 | | QSLTAS | | | SLI | | | | | WB |
| | | | LIY | | | | | | | | |
| 790 | DRB1_ | 940 | DPETFY | 5 | 1251 | YQSLTA | 0.86 | Sequence | 0.447487 | 2.02 | <= |
| | 0103 | | QSLTAS | | | SLI | | | | | WB |
| | | | LIY | | | | | | | | |
| 790 | DRB1_ | 940 | DPETFY | 4 | 1250 | FYQSLT | 0.92 | Sequence | 0.753529 | 0.53 | <= |
| | 0701 | | QSLTAS | | | ASL | | | | | SB |
| | | | LIY | | | | | | | | |
| 790 | DRB1_ | 940 | DPETFY | 4 | 1250 | FYQSLT | 0.653 | Sequence | 0.803348 | 0.22 | <= |
| | 0901 | | QSLTAS | | | ASL | | | | | SB |
| | | | LIY | | | | | | | | |
| 790 | DRB1_ | 940 | DPETFY | 5 | 1251 | YQSLTA | 0.78 | Sequence | 0.58271 | 2.09 | <= |
| | 1001 | | QSLTAS | | | SLI | | | | | WB |
| | | | LIY | | | | | | | | |
| 790 | DRB1_ | 940 | DPETFY | 5 | 1251 | YQSLTA | 0.767 | Sequence | 0.423458 | 3.42 | <= |
| | 1601 | | QSLTAS | | | SLI | | | | | WB |
| | | | LIY | | | | | | | | |
| 791 | DRB1_ | 941 | PETFYQ | 4 | 1251 | YQSLTA | 1 | Sequence | 0.967605 | 0.11 | <= |
| | 0101 | | SLTASL | | | SLI | | | | | SB |
| | | | IYD | | | | | | | | |
| 791 | DRB1_ | 941 | PETFYQ | 4 | 1251 | YQSLTA | 0.98 | Sequence | 0.786412 | 0.97 | <= |
| | 0102 | | SLTASL | | | SLI | | | | | SB |
| | | | IYD | | | | | | | | |
| 791 | DRB1_ | 941 | PETFYQ | 4 | 1251 | YQSLTA | 0.907 | Sequence | 0.687944 | 0.65 | <= |
| | 0103 | | SLTASL | | | SLI | | | | | SB |
| | | | IYD | | | | | | | | |
| 791 | DRB1_ | 941 | PETFYQ | 3 | 1250 | FYQSLT | 0.833 | Sequence | 0.849095 | 0.27 | <= |
| | 0701 | | SLTASL | | | ASL | | | | | SB |
| | | | IYD | | | | | | | | |
| 791 | DRB1_ | 941 | PETFYQ | 3 | 1250 | FYQSLT | 0.587 | Sequence | 0.88013 | 0.06 | <= |
| | 0901 | | SLTASL | | | ASL | | | | | SB |
| | | | IYD | | | | | | | | |
| 791 | DRB1_ | 941 | PETFYQ | 4 | 1251 | YQSLTA | 0.847 | Sequence | 0.772003 | 0.66 | <= |
| | 1001 | | SLTASL | | | SLI | | | | | SB |
| | | | IYD | | | | | | | | |
| 791 | DRB1_ | 941 | PETFYQ | 4 | 1251 | YQSLTA | 0.873 | Sequence | 0.70993 | 0.87 | <= |
| | 1601 | | SLTASL | | | SLI | | | | | SB |
| | | | IYD | | | | | | | | |
| 791 | DRB1_ | 941 | PETFYQ | 3 | 1250 | FYQSLT | 0.58 | Sequence | 0.316512 | 3.98 | <= |
| | 0803 | | SLTASL | | | ASL | | | | | WB |
| | | | IYD | | | | | | | | |
| 792 | DRB1_ | 942 | ETFYQS | 3 | 1251 | YQSLTA | 1 | Sequence | 0.971519 | 0.09 | <= |
| | 0101 | | LTASLI | | | SLI | | | | | SB |
| | | | YDY | | | | | | | | |
| 792 | DRB1_ | 942 | ETFYQS | 3 | 1251 | YQSLTA | 1 | Sequence | 0.800079 | 0.89 | <= |
| | 0102 | | LTASLI | | | SLI | | | | | SB |
| | | | YDY | | | | | | | | |
| 792 | DRB1_ | 942 | ETFYQS | 3 | 1251 | YQSLTA | 1 | Sequence | 0.706138 | 0.6 | <= |
| | 0103 | | LTASLI | | | SLI | | | | | SB |
| | | | YDY | | | | | | | | |
| 792 | DRB1_ | 942 | ETFYQS | 3 | 1251 | YQSLTA | 0.547 | Sequence | 0.632252 | 0.99 | <= |
| | 0701 | | LTASLI | | | SLI | | | | | SB |
| | | | YDY | | | | | | | | |
| 792 | DRB1_ | 942 | ETFYQS | 3 | 1251 | YQSLTA | 0.68 | Sequence | 0.799776 | 0.23 | <= |
| | 0901 | | LTASLI | | | SLI | | | | | SB |
| | | | YDY | | | | | | | | |
| 792 | DRB1_ | 942 | ETFYQS | 3 | 1251 | YQSLTA | 0.987 | Sequence | 0.750689 | 0.78 | <= |
| | 1001 | | LTASLI | | | SLI | | | | | SB |
| | | | YDY | | | | | | | | |
| 792 | DRB1_ | 942 | ETFYQS | 3 | 1251 | YQSLTA | 0.993 | Sequence | 0.711729 | 0.86 | <= |
| | 1601 | | LTASLI | | | SLI | | | | | SB |
| | | | YDY | | | | | | | | |
| 793 | DRB1_ | 943 | TFYQSL | 2 | 1251 | YQSLTA | 1 | Sequence | 0.842931 | 0.67 | <= |
| | 0101 | | TASLIY | | | SLI | | | | | SB |
| | | | DYD | | | | | | | | |
| 793 | DRB1_ | 943 | TFYQSL | 2 | 1251 | YQSLTA | 1 | Sequence | 0.534408 | 2.73 | <= |
| | 0102 | | TASLIY | | | SLI | | | | | WB |
| | | | DYD | | | | | | | | |
| 793 | DRB1_ | 943 | TFYQSL | 2 | 1251 | YQSLTA | 0.993 | Sequence | 0.402507 | 2.43 | <= |
| | 0103 | | TASLIY | | | SLI | | | | | WB |
| | | | DYD | | | | | | | | |
| 793 | DRB1_ | 943 | TFYQSL | 2 | 1251 | YQSLTA | 0.82 | Sequence | 0.54378 | 1.74 | <= |
| | 0901 | | TASLIY | | | SLI | | | | | WB |
| | | | DYD | | | | | | | | |
| 793 | DRB1_ | 943 | TFYQSL | 2 | 1251 | YQSLTA | 0.98 | Sequence | 0.468986 | 3.44 | <= |
| | 1001 | | TASLIY | | | SLI | | | | | WB |
| | | | DYD | | | | | | | | |
| 793 | DRB1_ | 943 | TFYQSL | 2 | 1251 | YQSLTA | 0.987 | Sequence | 0.334337 | 4.76 | <= |
| | 1601 | | TASLIY | | | SLI | | | | | WB |
| | | | DYD | | | | | | | | |
| 794 | DRB1_ | 944 | FYQSLT | 1 | 1251 | YQSLTA | 1 | Sequence | 0.36983 | 3.71 | <= |
| | 0101 | | ASLIYD | | | SLI | | | | | WB |
| | | | YDY | | | | | | | | |
| 801 | DRB1_ | 945 | SLIYDY | 5 | 1252 | YDYFTT | 0.993 | Sequence | 0.385889 | 3.21 | <= |
| | 0401 | | DYFTTE | | | ELS | | | | | WB |
| | | | LSG | | | | | | | | |
| 801 | DRB1_ | 945 | SLIYDY | 5 | 1252 | YDYFTT | 1 | Sequence | 0.386942 | 2.82 | <= |
| | 0405 | | DYFTTE | | | ELS | | | | | WB |
| | | | LSG | | | | | | | | |
| 801 | DRB1_ | 945 | SLIYDY | 5 | 1252 | YDYFTT | 0.993 | Sequence | 0.595377 | 1.68 | <= |
| | 0408 | | DYFTTE | | | ELS | | | | | WB |
| | | | LSG | | | | | | | | |
| 802 | DRB1_ | 946 | LIYDYD | 4 | 1252 | YDYFTT | 1 | Sequence | 0.62502 | 1.21 | <= |
| | 0401 | | YFTTEL | | | ELS | | | | | WB |
| | | | SGE | | | | | | | | |
| 802 | DRB1_ | 946 | LIYDYD | 4 | 1252 | YDYFTT | 0.993 | Sequence | 0.257969 | 4.86 | <= |
| | 0402 | | YFTTEL | | | ELS | | | | | WB |
| | | | SGE | | | | | | | | |
| 802 | DRB1_ | 946 | LIYDYD | 4 | 1252 | YDYFTT | 1 | Sequence | 0.519676 | 2.69 | <= |
| | 0403 | | YFTTEL | | | ELS | | | | | WB |
| | | | SGE | | | | | | | | |
| 802 | DRB1_ | 946 | LIYDYD | 4 | 1252 | YDYFTT | 1 | Sequence | 0.454805 | 3.18 | <= |
| | 0404 | | YFTTEL | | | ELS | | | | | WB |
| | | | SGE | | | | | | | | |
| 802 | DRB1_ | 946 | LIYDYD | 4 | 1252 | YDYFTT | 1 | Sequence | 0.569098 | 1.43 | <= |
| | 0405 | | YFTTEL | | | ELS | | | | | WB |
| | | | SGE | | | | | | | | |
| 802 | DRB1_ | 946 | LIYDYD | 4 | 1252 | YDYFTT | 0.993 | Sequence | 0.547666 | 2.43 | <= |
| | 1001 | | YFTTEL | | | ELS | | | | | WB |
| | | | SGE | | | | | | | | |
| 802 | DRB1_ | 946 | LIYDYD | 4 | 1252 | YDYFTT | 1 | Sequence | 0.499702 | 2.5 | <= |
| | 1601 | | YFTTEL | | | ELS | | | | | WB |
| | | | SGE | | | | | | | | |
| 802 | DRB1_ | 946 | LIYDYD | 4 | 1252 | YDYFTT | 1 | Sequence | 0.787699 | 0.56 | <= |
| | 0408 | | YFTTEL | | | ELS | | | | | SB |
| | | | SGE | | | | | | | | |
| 803 | DRB1_ | 947 | IYDYDY | 3 | 1252 | YDYFTT | 0.987 | Sequence | 0.527352 | 1.89 | <= |
| | 0401 | | FTTELS | | | ELS | | | | | WB |
| | | | GEL | | | | | | | | |
| 803 | DRB1_ | 947 | IYDYDY | 3 | 1252 | YDYFTT | 0.993 | Sequence | 0.415799 | 4.22 | <= |
| | 0403 | | FTTELS | | | ELS | | | | | WB |
| | | | GEL | | | | | | | | |
| 803 | DRB1_ | 947 | IYDYDY | 3 | 1252 | YDYFTT | 0.993 | Sequence | 0.385416 | 4.06 | <= |
| | 0404 | | FTTELS | | | ELS | | | | | WB |
| | | | GEL | | | | | | | | |
| 803 | DRB1_ | 947 | IYDYDY | 3 | 1252 | YDYFTT | 0.973 | Sequence | 0.456282 | 2.18 | <= |
| | 0405 | | FTTELS | | | ELS | | | | | WB |
| | | | GEL | | | | | | | | |
| 803 | DRB1_ | 947 | IYDYDY | 3 | 1252 | YDYFTT | 0.98 | Sequence | 0.462155 | 3.55 | <= |
| | 1001 | | FTTELS | | | ELS | | | | | WB |
| | | | GEL | | | | | | | | |
| 803 | DRB1_ | 947 | IYDYDY | 3 | 1252 | YDYFTT | 0.987 | Sequence | 0.396435 | 3.78 | <= |
| | 1601 | | FTTELS | | | ELS | | | | | WB |
| | | | GEL | | | | | | | | |
| 803 | DRB1_ | 947 | IYDYDY | 3 | 1252 | YDYFTT | 0.993 | Sequence | 0.727356 | 0.83 | <= |
| | 0408 | | FTTELS | | | ELS | | | | | SB |
| | | | GEL | | | | | | | | |
| 804 | DRB1_ | 948 | YDYDYF | 2 | 1252 | YDYFTT | 0.873 | Sequence | 0.325566 | 4.08 | <= |
| | 0401 | | TTELSG | | | ELS | | | | | WB |
| | | | ELK | | | | | | | | |
| 804 | DRB1_ | 948 | YDYDYF | 2 | 1252 | YDYFTT | 0.8 | Sequence | 0.297593 | 4.01 | <= |
| | 0405 | | TTELSG | | | ELS | | | | | WB |
| | | | ELK | | | | | | | | |
| 804 | DRB1_ | 948 | YDYDYF | 2 | 1252 | YDYFTT | 0.98 | Sequence | 0.510154 | 2.46 | <= |
| | 0408 | | TTELSG | | | ELS | | | | | WB |
| | | | ELK | | | | | | | | |
| 805 | DRB1_ | 949 | DYDYFT | 4 | 1253 | FTTELS | 0.853 | Sequence | 0.381131 | 4.27 | <= |
| | 0901 | | TELSGE | | | GEL | | | | | WB |
| | | | LKN | | | | | | | | |
| 806 | DRB1_ | 950 | YDYFTT | 3 | 1253 | FTTELS | 1 | Sequence | 0.316997 | 4.29 | <= |
| | 0101 | | ELSGEL | | | GEL | | | | | WB |
| | | | KNS | | | | | | | | |
| 806 | DRB1_ | 950 | YDYFTT | 3 | 1253 | FTTELS | 0.993 | Sequence | 0.397003 | 3.93 | <= |
| | 0901 | | ELSGEL | | | GEL | | | | | WB |
| | | | KNS | | | | | | | | |
| 809 | DRB1_ | 951 | FTTELS | 4 | 1254 | LSGELK | 1 | Sequence | 0.266738 | 4.26 | <= |
| | 0301 | | GELKNS | | | NSF | | | | | WB |
| | | | FED | | | | | | | | |
| 809 | DRB1_ | 951 | FTTELS | 4 | 1254 | LSGELK | 1 | Sequence | 0.335554 | 4.24 | <= |
| | 1401 | | GELKNS | | | NSF | | | | | WB |
| | | | FED | | | | | | | | |
| 809 | DRB1_ | 951 | FTTELS | 4 | 1254 | LSGELK | 1 | Sequence | 0.335554 | 4.24 | <= |
| | 1454 | | GELKNS | | | NSF | | | | | WB |
| | | | FED | | | | | | | | |
| 810 | DRB1_ | 952 | TTELSG | 3 | 1254 | LSGELK | 1 | Sequence | 0.264314 | 4.3 | <= |
| | 0301 | | ELKNSF | | | NSF | | | | | WB |
| | | | EDI | | | | | | | | |
| 810 | DRB1_ | 952 | TTELSG | 3 | 1254 | LSGELK | 1 | Sequence | 0.321219 | 4.59 | <= |
| | 1401 | | ELKNSF | | | NSF | | | | | WB |
| | | | EDI | | | | | | | | |
| 810 | DRB1_ | 952 | TTELSG | 3 | 1254 | LSGELK | 1 | Sequence | 0.321219 | 4.59 | <= |
| | 1454 | | ELKNSF | | | NSF | | | | | WB |
| | | | EDI | | | | | | | | |
| 816 | DRB1_ | 953 | ELKNSF | 5 | 1255 | FEDIKA | 1 | Sequence | 0.41546 | 2.52 | <= |
| | 0405 | | EDIKAS | | | SSE | | | | | WB |
| | | | SEL | | | | | | | | |
| 817 | DRB1_ | 954 | LKNSFE | 4 | 1255 | FEDIKA | 1 | Sequence | 0.507643 | 2.85 | <= |
| | 0403 | | DIKASS | | | SSE | | | | | WB |
| | | | ELK | | | | | | | | |
| 817 | DRB1_ | 954 | LKNSFE | 4 | 1255 | FEDIKA | 1 | Sequence | 0.637646 | 1.1 | <= |
| | 0405 | | DIKASS | | | SSE | | | | | WB |
| | | | ELK | | | | | | | | |
| 817 | DRB1_ | 954 | LKNSFE | 4 | 1255 | FEDIKA | 1 | Sequence | 0.238508 | 5 | <= |
| | 0701 | | DIKASS | | | SSE | | | | | WB |
| | | | ELK | | | | | | | | |
| 817 | DRB1_ | 954 | LKNSFE | 4 | 1255 | FEDIKA | 0.993 | Sequence | 0.549236 | 2.84 | <= |
| | 0801 | | DIKASS | | | SSE | | | | | WB |
| | | | ELK | | | | | | | | |
| 817 | DRB1_ | 954 | LKNSFE | 4 | 1255 | FEDIKA | 1 | Sequence | 0.530595 | 2.63 | <= |
| | 1001 | | DIKASS | | | SSE | | | | | WB |
| | | | ELK | | | | | | | | |
| 817 | DRB1_ | 954 | LKNSFE | 4 | 1255 | FEDIKA | 0.987 | Sequence | 0.426825 | 3.19 | <= |
| | 1303 | | DIKASS | | | SSE | | | | | WB |
| | | | ELK | | | | | | | | |
| 817 | DRB1_ | 954 | LKNSFE | 4 | 1255 | FEDIKA | 0.993 | Sequence | 0.357013 | 2.99 | <= |
| | 0803 | | DIKASS | | | SSE | | | | | WB |
| | | | ELK | | | | | | | | |
| 818 | DRB1_ | 955 | KNSFED | 3 | 1255 | FEDIKA | 0.993 | Sequence | 0.445139 | 3.71 | <= |
| | 0403 | | IKASSE | | | SSE | | | | | WB |
| | | | LKL | | | | | | | | |
| 818 | DRB1_ | 955 | KNSFED | 3 | 1255 | FEDIKA | 1 | Sequence | 0.571498 | 1.42 | <= |
| | 0405 | | IKASSE | | | SSE | | | | | WB |
| | | | LKL | | | | | | | | |
| 818 | DRB1_ | 955 | KNSFED | 3 | 1255 | FEDIKA | 0.7 | Sequence | 0.246353 | 4.84 | <= |
| | 0701 | | IKASSE | | | SSE | | | | | WB |
| | | | LKL | | | | | | | | |
| 818 | DRB1_ | 955 | KNSFED | 3 | 1255 | FEDIKA | 1 | Sequence | 0.474601 | 3.98 | <= |
| | 0801 | | IKASSE | | | SSE | | | | | WB |
| | | | LKL | | | | | | | | |
| 818 | DRB1_ | 955 | KNSFED | 3 | 1255 | FEDIKA | 0.6 | Sequence | 0.378169 | 4.33 | <= |
| | 0901 | | IKASSE | | | SSE | | | | | WB |
| | | | LKL | | | | | | | | |
| 818 | DRB1_ | 955 | KNSFED | 3 | 1255 | FEDIKA | 1 | Sequence | 0.493686 | 3.09 | <= |
| | 1001 | | IKASSE | | | SSE | | | | | WB |
| | | | LKL | | | | | | | | |
| 818 | DRB1_ | 955 | KNSFED | 3 | 1255 | FEDIKA | 0.98 | Sequence | 0.364392 | 3.99 | <= |
| | 1303 | | IKASSE | | | SSE | | | | | WB |
| | | | LKL | | | | | | | | |
| 818 | DRB1_ | 955 | KNSFED | 3 | 1255 | FEDIKA | 0.953 | Sequence | 0.305915 | 4.3 | <= |
| | 0803 | | IKASSE | | | SSE | | | | | WB |
| | | | LKL | | | | | | | | |
| 819 | DRB1_ | 956 | NSFEDI | 5 | 1256 | IKASSE | 0.947 | Sequence | 0.323048 | 3.53 | <= |
| | 0701 | | KASSEL | | | LKL | | | | | WB |
| | | | KLS | | | | | | | | |
| 819 | DRB1_ | 956 | NSFEDI | 5 | 1256 | IKASSE | 0.867 | Sequence | 0.438838 | 3.16 | <= |
| | 0901 | | KASSEL | | | LKL | | | | | WB |
| | | | KLS | | | | | | | | |
| 819 | DRB1_ | 956 | NSFEDI | 5 | 1256 | IKASSE | 0.987 | Sequence | 0.440811 | 2.37 | <= |
| | 1401 | | KASSEL | | | LKL | | | | | WB |
| | | | KLS | | | | | | | | |
| 819 | DRB1_ | 956 | NSFEDI | 5 | 1256 | IKASSE | 0.987 | Sequence | 0.440811 | 2.37 | <= |
| | 1454 | | KASSEL | | | LKL | | | | | WB |
| | | | KLS | | | | | | | | |
| 820 | DRB1_ | 957 | SFEDIK | 4 | 1256 | IKASSE | 1 | Sequence | 0.313951 | 3.67 | <= |
| | 0701 | | ASSELK | | | LKL | | | | | WB |
| | | | LSL | | | | | | | | |
| 820 | DRB1_ | 957 | SFEDIK | 4 | 1256 | IKASSE | 0.98 | Sequence | 0.413445 | 3.61 | <= |
| | 0901 | | ASSELK | | | LKL | | | | | WB |
| | | | LSL | | | | | | | | |
| 820 | DRB1_ | 957 | SFEDIK | 4 | 1256 | IKASSE | 1 | Sequence | 0.454336 | 2.21 | <= |
| | 1401 | | ASSELK | | | LKL | | | | | WB |
| | | | LSL | | | | | | | | |
| 820 | DRB1_ | 957 | SFEDIK | 4 | 1256 | IKASSE | 1 | Sequence | 0.454336 | 2.21 | <= |
| | 1454 | | ASSELK | | | LKL | | | | | WB |
| | | | LSL | | | | | | | | |
| 821 | DRB1_ | 958 | FEDIKA | 3 | 1256 | IKASSE | 1 | Sequence | 0.425041 | 2.37 | <= |
| | 0701 | | SSELKL | | | LKL | | | | | WB |
| | | | SLD | | | | | | | | |
| 821 | DRB1_ | 958 | FEDIKA | 3 | 1256 | IKASSE | 0.993 | Sequence | 0.500974 | 2.24 | <= |
| | 0901 | | SSELKL | | | LKL | | | | | WB |
| | | | SLD | | | | | | | | |
| 821 | DRB1_ | 958 | FEDIKA | 3 | 1256 | IKASSE | 1 | Sequence | 0.552066 | 1.28 | <= |
| | 1401 | | SSELKL | | | LKL | | | | | WB |
| | | | SLD | | | | | | | | |
| 821 | DRB1_ | 958 | FEDIKA | 3 | 1256 | IKASSE | 1 | Sequence | 0.552066 | 1.28 | <= |
| | 1454 | | SSELKL | | | LKL | | | | | WB |
| | | | SLD | | | | | | | | |
| 822 | DRB1_ | 959 | EDIKAS | 2 | 1256 | IKASSE | 1 | Sequence | 0.262468 | 4.51 | <= |
| | 0701 | | SELKLS | | | LKL | | | | | WB |
| | | | LDF | | | | | | | | |
| 822 | DRB1_ | 959 | EDIKAS | 2 | 1256 | IKASSE | 1 | Sequence | 0.379733 | 4.3 | <= |
| | 0901 | | SELKLS | | | LKL | | | | | WB |
| | | | LDF | | | | | | | | |
| 822 | DRB1_ | 959 | EDIKAS | 2 | 1256 | IKASSE | 0.987 | Sequence | 0.395755 | 3.01 | <= |
| | 1401 | | SELKLS | | | LKL | | | | | WB |
| | | | LDF | | | | | | | | |
| 822 | DRB1_ | 959 | EDIKAS | 2 | 1256 | IKASSE | 0.987 | Sequence | 0.395755 | 3.01 | <= |
| | 1454 | | SELKLS | | | LKL | | | | | WB |
| | | | LDF | | | | | | | | |
| 829 | DRB1_ | 960 | ELKLSL | 3 | 1257 | LSLDFP | 1 | Sequence | 0.241385 | 4.67 | <= |
| | 0301 | | DFPYLL | | | YLL | | | | | WB |
| | | | QEA | | | | | | | | |
| 829 | DRB1_ | 960 | ELKLSL | 3 | 1257 | LSLDFP | 0.987 | Sequence | 0.102055 | 4.75 | <= |
| | 0305 | | DFPYLL | | | YLL | | | | | WB |
| | | | QEA | | | | | | | | |
| 833 | DRB1_ | 961 | SLDFPY | 5 | 1258 | YLLQEA | 0.993 | Sequence | 0.44501 | 3.83 | <= |
| | 1001 | | LLQEAG | | | GIG | | | | | WB |
| | | | IGI | | | | | | | | |
| 834 | DRB1_ | 962 | LDFPYL | 4 | 1258 | YLLQEA | 0.773 | Sequence | 0.578145 | 2.01 | <= |
| | 0101 | | LQEAGI | | | GIG | | | | | WB |
| | | | GIK | | | | | | | | |
| 834 | DRB1_ | 962 | LDFPYL | 5 | 1259 | LLQEAG | 0.693 | Sequence | 0.55345 | 2.55 | <= |
| | 0102 | | LQEAGI | | | IGI | | | | | WB |
| | | | GIK | | | | | | | | |
| 834 | DRB1_ | 962 | LDFPYL | 4 | 1258 | YLLQEA | 0.733 | Sequence | 0.285245 | 4.04 | <= |
| | 0103 | | LQEAGI | | | GIG | | | | | WB |
| | | | GIK | | | | | | | | |
| 834 | DRB1_ | 962 | LDFPYL | 4 | 1258 | YLLQEA | 0.987 | Sequence | 0.315196 | 4.26 | <= |
| | 0401 | | LQEAGI | | | GIG | | | | | WB |
| | | | GIK | | | | | | | | |
| 834 | DRB1_ | 962 | LDFPYL | 4 | 1258 | YLLQEA | 0.993 | Sequence | 0.665721 | 1.37 | <= |
| | 1001 | | LQEAGI | | | GIG | | | | | WB |
| | | | GIK | | | | | | | | |
| 834 | DRB1_ | 962 | LDFPYL | 4 | 1258 | YLLQEA | 0.993 | Sequence | 0.374548 | 4.05 | <= |
| | 0408 | | LQEAGI | | | GIG | | | | | WB |
| | | | GIK | | | | | | | | |
| 835 | DRB1_ | 963 | DFPYLL | 3 | 1258 | YLLQEA | 0.527 | Sequence | 0.702032 | 1.34 | <= |
| | 0101 | | QEAGIG | | | GIG | | | | | WB |
| | | | IKY | | | | | | | | |
| 835 | DRB1_ | 963 | DFPYLL | 4 | 1259 | LLQEAG | 0.82 | Sequence | 0.726212 | 1.32 | <= |
| | 0102 | | QEAGIG | | | IGI | | | | | WB |
| | | | IKY | | | | | | | | |
| 835 | DRB1_ | 963 | DFPYLL | 3 | 1258 | YLLQEA | 0.673 | Sequence | 0.357847 | 2.93 | <= |
| | 0103 | | QEAGIG | | | GIG | | | | | WB |
| | | | IKY | | | | | | | | |
| 835 | DRB1_ | 963 | DFPYLL | 3 | 1258 | YLLQEA | 0.973 | Sequence | 0.340169 | 3.85 | <= |
| | 0401 | | QEAGIG | | | GIG | | | | | WB |
| | | | IKY | | | | | | | | |
| 835 | DRB1_ | 963 | DFPYLL | 3 | 1258 | YLLQEA | 0.987 | Sequence | 0.685279 | 1.21 | <= |
| | 1001 | | QEAGIG | | | GIG | | | | | WB |
| | | | IKY | | | | | | | | |
| 835 | DRB1_ | 963 | DFPYLL | 3 | 1258 | YLLQEA | 0.987 | Sequence | 0.396995 | 3.73 | <= |
| | 0408 | | QEAGIG | | | GIG | | | | | WB |
| | | | IKY | | | | | | | | |
| 836 | DRB1_ | 964 | FPYLLQ | 3 | 1259 | LLQEAG | 0.873 | Sequence | 0.607916 | 1.83 | <= |
| | 0101 | | EAGIGI | | | IGI | | | | | WB |
| | | | KYY | | | | | | | | |
| 836 | DRB1_ | 964 | FPYLLQ | 3 | 1259 | LLQEAG | 0.98 | Sequence | 0.73862 | 1.24 | <= |
| | 0102 | | EAGIGI | | | IGI | | | | | WB |
| | | | KYY | | | | | | | | |
| 836 | DRB1_ | 964 | FPYLLQ | 3 | 1259 | LLQEAG | 0.787 | Sequence | 0.242059 | 4.9 | <= |
| | 0103 | | EAGIGI | | | IGI | | | | | WB |
| | | | KYY | | | | | | | | |
| 836 | DRB1_ | 964 | FPYLLQ | 2 | 1258 | YLLQEA | 0.913 | Sequence | 0.417319 | 4.31 | <= |
| | 1001 | | EAGIGI | | | GIG | | | | | WB |
| | | | KYY | | | | | | | | |
| 837 | DRB1_ | 965 | PYLLQE | 2 | 1259 | LLQEAG | 0.953 | Sequence | 0.401894 | 3.39 | <= |
| | 0101 | | AGIGIK | | | IGI | | | | | WB |
| | | | YYK | | | | | | | | |
| 837 | DRB1_ | 965 | PYLLQE | 2 | 1259 | LLQEAG | 0.993 | Sequence | 0.585608 | 2.29 | <= |
| | 0102 | | AGIGIK | | | IGI | | | | | WB |
| | | | YYK | | | | | | | | |
| 841 | DRB1_ | 966 | QEAGIG | 6 | 1260 | IKYYKK | 0.973 | Sequence | 0.707233 | 1.25 | <= |
| | 1301 | | IKYYKK | | | FKE | | | | | WB |
| | | | FKE | | | | | | | | |
| 841 | DRB1_ | 966 | QEAGIG | 6 | 1260 | IKYYKK | 0.933 | Sequence | 0.204941 | 3.98 | <= |
| | 1503 | | IKYYKK | | | FKE | | | | | WB |
| | | | FKE | | | | | | | | |
| 842 | DRB1_ | 967 | EAGIGI | 5 | 1260 | IKYYKK | 0.993 | Sequence | 0.295595 | 4.02 | <= |
| | 0402 | | KYYKKF | | | FKE | | | | | WB |
| | | | KED | | | | | | | | |
| 842 | DRB1_ | 967 | EAGIGI | 5 | 1260 | IKYYKK | 0.98 | Sequence | 0.653957 | 1.68 | <= |
| | 0801 | | KYYKKF | | | FKE | | | | | WB |
| | | | KED | | | | | | | | |
| 842 | DRB1_ | 967 | EAGIGI | 5 | 1260 | IKYYKK | 0.987 | Sequence | 0.374579 | 4.55 | <= |
| | 1101 | | KYYKKF | | | FKE | | | | | WB |
| | | | KED | | | | | | | | |
| 842 | DRB1_ | 967 | EAGIGI | 5 | 1260 | IKYYKK | 0.993 | Sequence | 0.720788 | 1.4 | <= |
| | 1104 | | KYYKKF | | | FKE | | | | | WB |
| | | | KED | | | | | | | | |
| 842 | DRB1_ | 967 | EAGIGI | 5 | 1260 | IKYYKK | 0.813 | Sequence | 0.318621 | 3.77 | <= |
| | 1201 | | KYYKKF | | | FKE | | | | | WB |
| | | | KED | | | | | | | | |
| 842 | DRB1_ | 967 | EAGIGI | 5 | 1260 | IKYYKK | 0.993 | Sequence | 0.922536 | 0.06 | <= |
| | 1301 | | KYYKKF | | | FKE | | | | | SB |
| | | | KED | | | | | | | | |
| 842 | DRB1_ | 967 | EAGIGI | 5 | 1260 | IKYYKK | 0.993 | Sequence | 0.357081 | 3.76 | <= |
| | 1401 | | KYYKKF | | | FKE | | | | | WB |
| | | | KED | | | | | | | | |
| 842 | DRB1_ | 967 | EAGIGI | 5 | 1260 | IKYYKK | 0.98 | Sequence | 0.420662 | 2.2 | <= |
| | 1501 | | KYYKKF | | | FKE | | | | | WB |
| | | | KED | | | | | | | | |
| 842 | DRB1_ | 967 | EAGIGI | 5 | 1260 | IKYYKK | 0.973 | Sequence | 0.456922 | 1.17 | <= |
| | 1503 | | KYYKKF | | | FKE | | | | | WB |
| | | | KED | | | | | | | | |
| 842 | DRB1_ | 967 | EAGIGI | 5 | 1260 | IKYYKK | 0.993 | Sequence | 0.357081 | 3.76 | <= |
| | 1454 | | KYYKKF | | | FKE | | | | | WB |
| | | | KED | | | | | | | | |
| 842 | DRB1_ | 967 | EAGIGI | 5 | 1260 | IKYYKK | 0.987 | Sequence | 0.449595 | 2.93 | <= |
| | 1303 | | KYYKKF | | | FKE | | | | | WB |
| | | | KED | | | | | | | | |
| 842 | DRB1_ | 967 | EAGIGI | 5 | 1260 | IKYYKK | 0.96 | Sequence | 0.515797 | 0.88 | <= |
| | 1302 | | KYYKKF | | | FKE | | | | | SB |
| | | | KED | | | | | | | | |
| 842 | DRB1_ | 967 | EAGIGI | 5 | 1260 | IKYYKK | 0.967 | Sequence | 0.517544 | 0.97 | <= |
| | 0803 | | KYYKKF | | | FKE | | | | | SB |
| | | | KED | | | | | | | | |
| 843 | DRB1_ | 968 | AGIGIK | 4 | 1260 | IKYYKK | 0.993 | Sequence | 0.398235 | 2.5 | <= |
| | 0402 | | YYKKFK | | | FKE | | | | | WB |
| | | | EDA | | | | | | | | |
| 843 | DRB1_ | 968 | AGIGIK | 4 | 1260 | IKYYKK | 0.927 | Sequence | 0.749894 | 0.91 | <= |
| | 0801 | | YYKKFK | | | FKE | | | | | SB |
| | | | EDA | | | | | | | | |
| 843 | DRB1_ | 968 | AGIGIK | 4 | 1260 | IKYYKK | 0.9 | Sequence | 0.532191 | 2.77 | <= |
| | 1101 | | YYKKFK | | | FKE | | | | | WB |
| | | | EDA | | | | | | | | |
| 843 | DRB1_ | 968 | AGIGIK | 4 | 1260 | IKYYKK | 1 | Sequence | 0.822325 | 0.77 | <= |
| | 1104 | | YYKKFK | | | FKE | | | | | SB |
| | | | EDA | | | | | | | | |
| 843 | DRB1_ | 968 | AGIGIK | 4 | 1260 | IKYYKK | 0.907 | Sequence | 0.369631 | 2.82 | <= |
| | 1201 | | YYKKFK | | | FKE | | | | | WB |
| | | | EDA | | | | | | | | |
| 843 | DRB1_ | 968 | AGIGIK | 4 | 1260 | IKYYKK | 0.993 | Sequence | 0.946891 | 0.02 | <= |
| | 1301 | | YYKKFK | | | FKE | | | | | SB |
| | | | EDA | | | | | | | | |
| 843 | DRB1_ | 968 | AGIGIK | 4 | 1260 | IKYYKK | 0.987 | Sequence | 0.433893 | 2.46 | <= |
| | 1401 | | YYKKFK | | | FKE | | | | | WB |
| | | | EDA | | | | | | | | |
| 843 | DRB1_ | 968 | AGIGIK | 4 | 1260 | IKYYKK | 1 | Sequence | 0.529154 | 1.53 | <= |
| | 1501 | | YYKKFK | | | FKE | | | | | WB |
| | | | EDA | | | | | | | | |
| 843 | DRB1_ | 968 | AGIGIK | 4 | 1260 | IKYYKK | 0.987 | Sequence | 0.332547 | 4.79 | <= |
| | 1601 | | YYKKFK | | | FKE | | | | | WB |
| | | | EDA | | | | | | | | |
| 843 | DRB1_ | 968 | AGIGIK | 4 | 1260 | IKYYKK | 0.993 | Sequence | 0.552626 | 0.74 | <= |
| | 1503 | | YYKKFK | | | FKE | | | | | SB |
| | | | EDA | | | | | | | | |
| 843 | DRB1_ | 968 | AGIGIK | 4 | 1260 | IKYYKK | 0.987 | Sequence | 0.433893 | 2.46 | <= |
| | 1454 | | YYKKFK | | | FKE | | | | | WB |
| | | | EDA | | | | | | | | |
| 843 | DRB1_ | 968 | AGIGIK | 4 | 1260 | IKYYKK | 0.96 | Sequence | 0.548603 | 1.94 | <= |
| | 1303 | | YYKKFK | | | FKE | | | | | WB |
| | | | EDA | | | | | | | | |
| 843 | DRB1_ | 968 | AGIGIK | 4 | 1260 | IKYYKK | 0.987 | Sequence | 0.605692 | 0.37 | <= |
| | 1302 | | YYKKFK | | | FKE | | | | | SB |
| | | | EDA | | | | | | | | |
| 843 | DRB1_ | 968 | AGIGIK | 4 | 1260 | IKYYKK | 0.953 | Sequence | 0.597768 | 0.52 | <= |
| | 0803 | | YYKKFK | | | FKE | | | | | SB |
| | | | EDA | | | | | | | | |
| 844 | DRB1_ | 969 | GIGIKY | 3 | 1260 | IKYYKK | 0.987 | Sequence | 0.390573 | 2.59 | <= |
| | 0402 | | YKKFKE | | | FKE | | | | | WB |
| | | | DAM | | | | | | | | |
| 844 | DRB1_ | 969 | GIGIKY | 3 | 1260 | IKYYKK | 0.833 | Sequence | 0.757275 | 0.87 | <= |
| | 0801 | | YKKFKE | | | FKE | | | | | SB |
| | | | DAM | | | | | | | | |
| 844 | DRB1_ | 969 | GIGIKY | 3 | 1260 | IKYYKK | 0.673 | Sequence | 0.604824 | 2.16 | <= |
| | 1101 | | YKKFKE | | | FKE | | | | | WB |
| | | | DAM | | | | | | | | |
| 844 | DRB1_ | 969 | GIGIKY | 3 | 1260 | IKYYKK | 0.987 | Sequence | 0.819507 | 0.79 | <= |
| | 1104 | | YKKFKE | | | FKE | | | | | SB |
| | | | DAM | | | | | | | | |
| 844 | DRB1_ | 969 | GIGIKY | 3 | 1260 | IKYYKK | 0.947 | Sequence | 0.364166 | 2.91 | <= |
| | 1201 | | YKKFKE | | | FKE | | | | | WB |
| | | | DAM | | | | | | | | |
| 844 | DRB1_ | 969 | GIGIKY | 3 | 1260 | IKYYKK | 1 | Sequence | 0.946656 | 0.02 | <= |
| | 1301 | | YKKFKE | | | FKE | | | | | SB |
| | | | DAM | | | | | | | | |
| 844 | DRB1_ | 969 | GIGIKY | 3 | 1260 | IKYYKK | 0.967 | Sequence | 0.427843 | 2.53 | <= |
| | 1401 | | YKKFKE | | | FKE | | | | | WB |
| | | | DAM | | | | | | | | |
| 844 | DRB1_ | 969 | GIGIKY | 3 | 1260 | IKYYKK | 1 | Sequence | 0.522548 | 1.57 | <= |
| | 1501 | | YKKFKE | | | FKE | | | | | WB |
| | | | DAM | | | | | | | | |
| 844 | DRB1_ | 969 | GIGIKY | 3 | 1260 | IKYYKK | 0.96 | Sequence | 0.327146 | 4.89 | <= |
| | 1601 | | YKKFKE | | | FKE | | | | | WB |
| | | | DAM | | | | | | | | |
| 844 | DRB1_ | 969 | GIGIKY | 3 | 1260 | IKYYKK | 0.993 | Sequence | 0.545253 | 0.77 | <= |
| | 1503 | | YKKFKE | | | FKE | | | | | SB |
| | | | DAM | | | | | | | | |
| 844 | DRB1_ | 969 | GIGIKY | 3 | 1260 | IKYYKK | 0.967 | Sequence | 0.427843 | 2.53 | <= |
| | 1454 | | YKKFKE | | | FKE | | | | | WB |
| | | | DAM | | | | | | | | |
| 844 | DRB1_ | 969 | GIGIKY | 3 | 1260 | IKYYKK | 0.853 | Sequence | 0.551637 | 1.91 | <= |
| | 1303 | | YKKFKE | | | FKE | | | | | WB |
| | | | DAM | | | | | | | | |
| 844 | DRB1_ | 969 | GIGIKY | 3 | 1260 | IKYYKK | 0.96 | Sequence | 0.606178 | 0.37 | <= |
| | 1302 | | YKKFKE | | | FKE | | | | | SB |
| | | | DAM | | | | | | | | |
| 844 | DRB1_ | 969 | GIGIKY | 3 | 1260 | IKYYKK | 0.9 | Sequence | 0.603401 | 0.5 | <= |
| | 0803 | | YKKFKE | | | FKE | | | | | SB |
| | | | DAM | | | | | | | | |
| 845 | DRB1_ | 970 | IGIKYY | 2 | 1260 | IKYYKK | 0.54 | Sequence | 0.681791 | 1.45 | <= |
| | 0801 | | KKFKED | | | FKE | | | | | WB |
| | | | AMK | | | | | | | | |
| 845 | DRB1_ | 970 | IGIKYY | 4 | 1261 | YYKKFK | 0.78 | Sequence | 0.632703 | 1.95 | <= |
| | 1101 | | KKFKED | | | EDA | | | | | WB |
| | | | AMK | | | | | | | | |
| 845 | DRB1_ | 970 | IGIKYY | 2 | 1260 | IKYYKK | 0.833 | Sequence | 0.658273 | 1.87 | <= |
| | 1104 | | KKFKED | | | FKE | | | | | WB |
| | | | AMK | | | | | | | | |
| 845 | DRB1_ | 970 | IGIKYY | 2 | 1260 | IKYYKK | 0.72 | Sequence | 0.271634 | 4.96 | <= |
| | 1201 | | KKFKED | | | FKE | | | | | WB |
| | | | AMK | | | | | | | | |
| 845 | DRB1_ | 970 | IGIKYY | 2 | 1260 | IKYYKK | 0.993 | Sequence | 0.894642 | 0.14 | <= |
| | 1301 | | KKFKED | | | FKE | | | | | SB |
| | | | AMK | | | | | | | | |
| 845 | DRB1_ | 970 | IGIKYY | 2 | 1260 | IKYYKK | 0.92 | Sequence | 0.338225 | 2.89 | <= |
| | 1501 | | KKFKED | | | FKE | | | | | WB |
| | | | AMK | | | | | | | | |
| 845 | DRB1_ | 970 | IGIKYY | 2 | 1260 | IKYYKK | 0.873 | Sequence | 0.406168 | 1.47 | <= |
| | 1503 | | KKFKED | | | FKE | | | | | WB |
| | | | AMK | | | | | | | | |
| 845 | DRB1_ | 970 | IGIKYY | 2 | 1260 | IKYYKK | 0.547 | Sequence | 0.413288 | 3.34 | <= |
| | 1303 | | KKFKED | | | FKE | | | | | WB |
| | | | AMK | | | | | | | | |
| 845 | DRB1_ | 970 | IGIKYY | 2 | 1260 | IKYYKK | 0.8 | Sequence | 0.453665 | 1.53 | <= |
| | 1302 | | KKFKED | | | FKE | | | | | WB |
| | | | AMK | | | | | | | | |
| 845 | DRB1_ | 970 | IGIKYY | 2 | 1260 | IKYYKK | 0.647 | Sequence | 0.490823 | 1.19 | <= |
| | 0803 | | KKFKED | | | FKE | | | | | WB |
| | | | AMK | | | | | | | | |
| 846 | DRB1_ | 971 | GIKYYK | 3 | 1261 | YYKKFK | 0.633 | Sequence | 0.605132 | 2.18 | <= |
| | 0801 | | KFKEDA | | | EDA | | | | | WB |
| | | | MKN | | | | | | | | |
| 846 | DRB1_ | 971 | GIKYYK | 3 | 1261 | YYKKFK | 0.98 | Sequence | 0.658168 | 1.78 | <= |
| | 1101 | | KFKEDA | | | EDA | | | | | WB |
| | | | MKN | | | | | | | | |
| 846 | DRB1_ | 971 | GIKYYK | 1 | 1260 | IKYYKK | 0.473 | Sequence | 0.452454 | 4.05 | <= |
| | 1104 | | KFKEDA | | | FKE | | | | | WB |
| | | | MKN | | | | | | | | |
| 846 | DRB1_ | 971 | GIKYYK | 1 | 1260 | IKYYKK | 0.927 | Sequence | 0.719938 | 1.15 | <= |
| | 1301 | | KFKEDA | | | FKE | | | | | WB |
| | | | MKN | | | | | | | | |
| 846 | DRB1_ | 971 | GIKYYK | 1 | 1260 | IKYYKK | 0.56 | Sequence | 0.29996 | 2.42 | <= |
| | 1503 | | KFKEDA | | | FKE | | | | | WB |
| | | | MKN | | | | | | | | |
| 846 | DRB1_ | 971 | GIKYYK | 3 | 1261 | YYKKFK | 0.727 | Sequence | 0.332618 | 4.48 | <= |
| | 1303 | | KFKEDA | | | EDA | | | | | WB |
| | | | MKN | | | | | | | | |
| 846 | DRB1_ | 971 | GIKYYK | 1 | 1260 | IKYYKK | 0.467 | Sequence | 0.29978 | 4.9 | <= |
| | 1302 | | KFKEDA | | | FKE | | | | | WB |
| | | | MKN | | | | | | | | |
| 846 | DRB1_ | 971 | GIKYYK | 3 | 1261 | YYKKFK | 0.5 | Sequence | 0.390205 | 2.41 | <= |
| | 0803 | | KFKEDA | | | EDA | | | | | WB |
| | | | MKN | | | | | | | | |
| 847 | DRB1_ | 972 | IKYYKK | 3 | 1262 | YKKFKE | 0.94 | Sequence | 0.193933 | 4.27 | <= |
| | 1503 | | FKEDAM | | | DAM | | | | | WB |
| | | | KNS | | | | | | | | |
| 858 | DRB1_ | 973 | MKNSGI | 5 | 1263 | ISAVQS | 1 | Sequence | 0.721329 | 0.93 | <= |
| | 0403 | | SAVOSP | | | PLE | | | | | SB |
| | | | LEP | | | | | | | | |
| 858 | DRB1_ | 973 | MKNSGI | 5 | 1263 | ISAVQS | 1 | Sequence | 0.496291 | 2.74 | <= |
| | 0404 | | SAVQSP | | | PLE | | | | | WB |
| | | | LEP | | | | | | | | |
| 858 | DRB1_ | 973 | MKNSGI | 5 | 1263 | ISAVQS | 1 | Sequence | 0.708273 | 0.81 | <= |
| | 0405 | | SAVQSP | | | PLE | | | | | SB |
| | | | LEP | | | | | | | | |
| 858 | DRB1_ | 973 | MKNSGI | 5 | 1263 | ISAVQS | 1 | Sequence | 0.354869 | 4.38 | <= |
| | 0408 | | SAVQSP | | | PLE | | | | | WB |
| | | | LEP | | | | | | | | |
| 859 | DRB1_ | 974 | KNSGIS | 4 | 1263 | ISAVQS | 1 | Sequence | 0.371611 | 3.39 | <= |
| | 0401 | | AVQSPL | | | PLE | | | | | WB |
| | | | EPQ | | | | | | | | |
| 859 | DRB1_ | 974 | KNSGIS | 4 | 1263 | ISAVQS | 1 | Sequence | 0.28708 | 4.2 | <= |
| | 0402 | | AVQSPL | | | PLE | | | | | WB |
| | | | EPQ | | | | | | | | |
| 859 | DRB1_ | 974 | KNSGIS | 4 | 1263 | ISAVQS | 1 | Sequence | 0.831759 | 0.35 | <= |
| | 0403 | | AVQSPL | | | PLE | | | | | SB |
| | | | EPQ | | | | | | | | |
| 859 | DRB1_ | 974 | KNSGIS | 4 | 1263 | ISAVQS | 1 | Sequence | 0.723333 | 1.04 | <= |
| | 0404 | | AVQSPL | | | PLE | | | | | WB |
| | | | EPQ | | | | | | | | |
| 859 | DRB1_ | 974 | KNSGIS | 4 | 1263 | ISAVQS | 1 | Sequence | 0.844654 | 0.35 | <= |
| | 0405 | | AVQSPL | | | PLE | | | | | SB |
| | | | EPQ | | | | | | | | |
| 859 | DRB1_ | 974 | KNSGIS | 4 | 1263 | ISAVQS | 0.993 | Sequence | 0.308029 | 4.92 | <= |
| | 1401 | | AVQSPL | | | PLE | | | | | WB |
| | | | EPQ | | | | | | | | |
| 859 | DRB1_ | 974 | KNSGIS | 4 | 1263 | ISAVQS | 0.993 | Sequence | 0.308029 | 4.92 | <= |
| | 1454 | | AVQSPL | | | PLE | | | | | WB |
| | | | EPQ | | | | | | | | |
| 859 | DRB1_ | 974 | KNSGIS | 4 | 1263 | ISAVQS | 0.993 | Sequence | 0.374232 | 3.86 | <= |
| | 1303 | | AVQSPL | | | PLE | | | | | WB |
| | | | EPQ | | | | | | | | |
| 859 | DRB1_ | 974 | KNSGIS | 4 | 1263 | ISAVQS | 0.987 | Sequence | 0.358162 | 2.97 | <= |
| | 0803 | | AVQSPL | | | PLE | | | | | WB |
| | | | EPQ | | | | | | | | |
| 859 | DRB1_ | 974 | KNSGIS | 4 | 1263 | ISAVQS | 1 | Sequence | 0.600825 | 1.64 | <= |
| | 0408 | | AVQSPL | | | PLE | | | | | WB |
| | | | EPQ | | | | | | | | |
| 860 | DRB1_ | 975 | NSGISA | 3 | 1263 | ISAVQS | 1 | Sequence | 0.54113 | 1.79 | <= |
| | 0401 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.443831 | 2.01 | <= |
| | 0402 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.887746 | 0.16 | <= |
| | 0403 | | VQSPLE | | | PLE | | | | | SB |
| | | | PQK | | | | | | | | |
| 860 | DRB1_ | 975 | NSGISA | 3 | 1263 | ISAVQS | 1 | Sequence | 0.843026 | 0.46 | <= |
| | 0404 | | VQSPLE | | | PLE | | | | | SB |
| | | | PQK | | | | | | | | |
| 860 | DRB1_ | 975 | NSGISA | 3 | 1263 | ISAVQS | 1 | Sequence | 0.905216 | 0.18 | <= |
| | 0405 | | VQSPLE | | | PLE | | | | | SB |
| | | | PQK | | | | | | | | |
| 860 | DRB1_ | 975 | NSGISA | 3 | 1263 | ISAVQS | 1 | Sequence | 0.29103 | 4.02 | <= |
| | 0701 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.514476 | 3.34 | <= |
| | 0801 | | VQSPLE | | | PLE | | | | | WB |
| | | | PQK | | | | | | | | |
| 860 | DRB1_ | 975 | NSGISA | 3 | 1263 | ISAVQS | 0.993 | Sequence | 0.399574 | 2.95 | <= |
| | 1401 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.346357 | 2.82 | <= |
| | 1501 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.368526 | 4.18 | <= |
| | 1601 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.26128 | 2.93 | <= |
| | 1503 | | VQSPLE | | | PLE | | | | | WB |
| | | | PQK | | | | | | | | |
| 860 | DRB1_ | 975 | NSGISA | 3 | 1263 | ISAVQS | 0.993 | Sequence | 0.399574 | 2.95 | <= |
| | 1454 | | VQSPLE | | | PLE | | | | | WB |
| | | | PQK | | | | | | | | |
| 860 | DRB 1303 | 975 | NSGISA | 3 | 1263 | ISAVQS | 0.993 | Sequence | 0.49525 | 2.44 | <= |
| | | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.477241 | 1.3 | <= |
| | 0803 | | VQSPLE | | | PLE | | | | | WB |
| | | | PQK | | | | | | | | |
| 860 | DRB1_ | 975 | NSGISA | 3 | 1263 | ISAVQS | 1 | Sequence | 0.760891 | 0.68 | <= |
| | 0408 | | VQSPLE | | | PLE | | | | | SB |
| | | | PQK | | | | | | | | |
| 861 | DRB1_ | 976 | SGISAV | 2 | 1263 | ISAVQS | 1 | Sequence | 0.732436 | 0.86 | <= |
| | 0403 | | QSPLEP | | | PLE | | | | | SB |
| | | | QKP | | | | | | | | |
| 861 | DRB1_ | 976 | SGISAV | 2 | 1263 | ISAVQS | 1 | Sequence | 0.600536 | 1.85 | <= |
| | 0404 | | QSPLEP | | | PLE | | | | | WB |
| | | | QKP | | | | | | | | |
| 861 | DRB1_ | 976 | SGISAV | 2 | 1263 | ISAVQS | 1 | Sequence | 0.71083 | 0.8 | <= |
| | 0405 | | QSPLEP | | | PLE | | | | | SB |
| | | | QKP | | | | | | | | |
| 861 | DRB1_ | 976 | SGISAV | 2 | 1263 | ISAVQS | 1 | Sequence | 0.434052 | 3.26 | <= |
| | 0408 | | QSPLEP | | | PLE | | | | | WB |
| | | | QKP | | | | | | | | |
| 875 | DRB1_ | 977 | PSSPYK | 4 | 1264 | YKNLEM | 1 | Sequence | 0.49049 | 3.14 | <= |
| | 1001 | | NLEMSP | | | SPA | | | | | WB |
| | | | ALY | | | | | | | | |
| 876 | DRB1_ | 978 | SSPYKN | 3 | 1264 | YKNLEM | 1 | Sequence | 0.519856 | 2.76 | <= |
| | 1001 | | LEMSPA | | | SPA | | | | | WB |
| | | | LYY | | | | | | | | |
| 877 | DRB1_ | 979 | SPYKNL | 5 | 1265 | LEMSPA | 1 | Sequence | 0.330432 | 3.43 | <= |
| | 0301 | | EMSPAL | | | LYY | | | | | WB |
| | | | YYK | | | | | | | | |
| 877 | DRB1_ | 979 | SPYKNL | 5 | 1265 | LEMSPA | 1 | Sequence | 0.27312 | 4.33 | <= |
| | 0701 | | EMSPAL | | | LYY | | | | | WB |
| | | | YYK | | | | | | | | |
| 877 | DRB1_ | 979 | SPYKNL | 5 | 1265 | LEMSPA | 0.993 | Sequence | 0.57906 | 0.88 | <= |
| | 1201 | | EMSPAL | | | LYY | | | | | SB |
| | | | YYK | | | | | | | | |
| 877 | DRB1_ | 979 | SPYKNL | 5 | 1265 | LEMSPA | 0.993 | Sequence | 0.401401 | 2.92 | <= |
| | 1401 | | EMSPAL | | | LYY | | | | | WB |
| | | | YYK | | | | | | | | |
| 877 | DRB1_ | 979 | SPYKNL | 5 | 1265 | LEMSPA | 0.993 | Sequence | 0.401401 | 2.92 | <= |
| | 1454 | | EMSPAL | | | LYY | | | | | WB |
| | | | YYK | | | | | | | | |
| 877 | DRB1_ | 979 | SPYKNL | 5 | 1265 | LEMSPA | 0.987 | Sequence | 0.388515 | 2.49 | <= |
| | 1302 | | EMSPAL | | | LYY | | | | | WB |
| | | | YYK | | | | | | | | |
| 878 | DRB1_ | 980 | PYKNLE | 4 | 1265 | LEMSPA | 1 | Sequence | 0.402401 | 2.78 | <= |
| | 0301 | | MSPALY | | | LYY | | | | | WB |
| | | | YKI | | | | | | | | |
| 878 | DRB1_ | 980 | PYKNLE | 4 | 1265 | LEMSPA | 0.993 | Sequence | 0.109139 | 4.46 | <= |
| | 0305 | | MSPALY | | | LYY | | | | | WB |
| | | | YKI | | | | | | | | |
| 878 | DRB1_ | 980 | PYKNLE | 4 | 1265 | LEMSPA | 0.993 | Sequence | 0.303067 | 3.84 | <= |
| | 0701 | | MSPALY | | | LYY | | | | | WB |
| | | | YKI | | | | | | | | |
| 878 | DRB1_ | 980 | PYKNLE | 4 | 1265 | LEMSPA | 0.993 | Sequence | 0.629193 | 0.64 | <= |
| | 1201 | | MSPALY | | | LYY | | | | | SB |
| | | | YKI | | | | | | | | |
| 878 | DRB1_ | 980 | PYKNLE | 4 | 1265 | LEMSPA | 1 | Sequence | 0.43236 | 2.47 | <= |
| | 1401 | | MSPALY | | | LYY | | | | | WB |
| | | | YKI | | | | | | | | |
| 878 | DRB1_ | 980 | PYKNLE | 4 | 1265 | LEMSPA | 1 | Sequence | 0.43236 | 2.47 | <= |
| | 1454 | | MSPALY | | | LYY | | | | | WB |
| | | | YKI | | | | | | | | |
| 878 | DRB1_ | 980 | PYKNLE | 4 | 1265 | LEMSPA | 0.987 | Sequence | 0.183701 | 3.68 | <= |
| | 1402 | | MSPALY | | | LYY | | | | | WB |
| | | | YKI | | | | | | | | |
| 878 | DRB1_ | 980 | PYKNLE | 4 | 1265 | LEMSPA | 1 | Sequence | 0.445366 | 1.62 | <= |
| | 1302 | | MSPALY | | | LYY | | | | | WB |
| | | | YKI | | | | | | | | |
| 879 | DRB1_ | 981 | YKNLEM | 3 | 1265 | LEMSPA | 1 | Sequence | 0.479365 | 2.19 | <= |
| | 0301 | | SPALYY | | | LYY | | | | | WB |
| | | | KIE | | | | | | | | |
| 879 | DRB1_ | 981 | YKNLEM | 3 | 1265 | LEMSPA | 0.993 | Sequence | 0.148084 | 3.36 | <= |
| | 0305 | | SPALYY | | | LYY | | | | | WB |
| | | | KIE | | | | | | | | |
| 879 | DRB1_ | 981 | YKNLEM | 3 | 1265 | LEMSPA | 0.973 | Sequence | 0.364014 | 2.98 | <= |
| | 0701 | | SPALYY | | | LYY | | | | | WB |
| | | | KIE | | | | | | | | |
| 879 | DRB1_ | 981 | YKNLEM | 3 | 1265 | LEMSPA | 0.967 | Sequence | 0.395969 | 3.95 | <= |
| | 0901 | | SPALYY | | | LYY | | | | | WB |
| | | | KIE | | | | | | | | |
| 879 | DRB1_ | 981 | YKNLEM | 3 | 1265 | LEMSPA | 0.987 | Sequence | 0.690577 | 0.42 | <= |
| | 1201 | | SPALYY | | | LYY | | | | | SB |
| | | | KIE | | | | | | | | |
| 879 | DRB1_ | 981 | YKNLEM | 3 | 1265 | LEMSPA | 1 | Sequence | 0.504717 | 1.7 | <= |
| | 1401 | | SPALYY | | | LYY | | | | | WB |
| | | | KIE | | | | | | | | |
| 879 | DRB1_ | 981 | YKNLEM | 3 | 1265 | LEMSPA | 1 | Sequence | 0.504717 | 1.7 | <= |
| | 1454 | | SPALYY | | | LYY | | | | | WB |
| | | | KIE | | | | | | | | |
| 879 | DRB1_ | 981 | YKNLEM | 3 | 1265 | LEMSPA | 1 | Sequence | 0.230474 | 2.23 | <= |
| | 1402 | | SPALYY | | | LYY | | | | | WB |
| | | | KIE | | | | | | | | |
| 879 | DRB1_ | 981 | YKNLEM | 3 | 1265 | LEMSPA | 1 | Sequence | 0.511927 | 0.92 | <= |
| | 1302 | | SPALYY | | | LYY | | | | | SB |
| | | | KIE | | | | | | | | |
| 880 | DRB1_ | 982 | KNLEMS | 2 | 1265 | LEMSPA | 1 | Sequence | 0.331301 | 3.42 | <= |
| | 0301 | | PALYYK | | | LYY | | | | | WB |
| | | | IEP | | | | | | | | |
| 880 | DRB1_ | 982 | KNLEMS | 2 | 1265 | LEMSPA | 0.967 | Sequence | 0.564735 | 0.96 | <= |
| | 1201 | | PALYYK | | | LYY | | | | | SB |
| | | | IEP | | | | | | | | |
| 880 | DRB1_ | 982 | KNLEMS | 2 | 1265 | LEMSPA | 0.98 | Sequence | 0.342211 | 4.08 | <= |
| | 1401 | | PALYYK | | | LYY | | | | | WB |
| | | | IEP | | | | | | | | |
| 880 | DRB1_ | 982 | KNLEMS | 2 | 1265 | LEMSPA | 0.98 | Sequence | 0.342211 | 4.08 | <= |
| | 1454 | | PALYYK | | | LYY | | | | | WB |
| | | | IEP | | | | | | | | |
| 880 | DRB1_ | 982 | KNLEMS | 2 | 1265 | LEMSPA | 0.98 | Sequence | 0.372739 | 2.8 | <= |
| | 1302 | | PALYYK | | | LYY | | | | | WB |
| | | | IEP | | | | | | | | |
| 884 | DRB1_ | 983 | MSPALY | 5 | 1266 | YYKIEP | 1 | Sequence | 0.546857 | 2.21 | <= |
| | 0101 | | YKIEPR | | | RYL | | | | | WB |
| | | | YLD | | | | | | | | |
| 884 | DRB1_ | 983 | MSPALY | 5 | 1266 | YYKIEP | 0.993 | Sequence | 0.654061 | 0.77 | <= |
| | 0103 | | YKIEPR | | | RYL | | | | | SB |
| | | | YLD | | | | | | | | |
| 884 | DRB1_ | 983 | MSPALY | 5 | 1266 | YYKIEP | 1 | Sequence | 0.551936 | 1.44 | <= |
| | 0701 | | YKIEPR | | | RYL | | | | | WB |
| | | | YLD | | | | | | | | |
| 884 | DRB1_ | 983 | MSPALY | 6 | 1267 | YKIEPR | 0.833 | Sequence | 0.658667 | 1.64 | <= |
| | 0801 | | YKIEPR | | | YLD | | | | | WB |
| | | | YLD | | | | | | | | |
| 884 | DRB1_ | 983 | MSPALY | 5 | 1266 | YYKIEP | 0.993 | Sequence | 0.504786 | 2.19 | <= |
| | 0901 | | YKIEPR | | | RYL | | | | | WB |
| | | | YLD | | | | | | | | |
| 884 | DRB1_ | 983 | MSPALY | 5 | 1266 | YYKIEP | 0.993 | Sequence | 0.407099 | 4.5 | <= |
| | 1001 | | YKIEPR | | | RYL | | | | | WB |
| | | | YLD | | | | | | | | |
| 884 | DRB1_ | 983 | MSPALY | 4 | 1268 | LYYKIE | 0.733 | Sequence | 0.305742 | 4.07 | <= |
| | 1201 | | YKIEPR | | | PRY | | | | | WB |
| | | | YLD | | | | | | | | |
| 884 | DRB1_ | 983 | MSPALY | 4 | 1268 | LYYKIE | 0.84 | Sequence | 0.470809 | 4.17 | <= |
| | 1301 | | YKIEPR | | | PRY | | | | | WB |
| | | | YLD | | | | | | | | |
| 884 | DRB1_ | 983 | MSPALY | 5 | 1266 | YYKIEP | 0.92 | Sequence | 0.350981 | 4.45 | <= |
| | 1601 | | YKIEPR | | | RYL | | | | | WB |
| | | | YLD | | | | | | | | |
| 884 | DRB1_ | 983 | MSPALY | 6 | 1267 | YKIEPR | 0.993 | Sequence | 0.854742 | 0.33 | <= |
| | 1303 | | YKIEPR | | | YLD | | | | | SB |
| | | | YLD | | | | | | | | |
| 884 | DRB1_ | 983 | MSPALY | 6 | 1267 | YKIEPR | 0.673 | Sequence | 0.458766 | 1.48 | <= |
| | 0803 | | YKIEPR | | | YLD | | | | | WB |
| | | | YLD | | | | | | | | |
| 885 | DRB1_ | 984 | SPALYY | 4 | 1266 | YYKIEP | 1 | Sequence | 0.720726 | 1.24 | <= |
| | 0101 | | KIEPRY | | | RYL | | | | | WB |
| | | | LDY | | | | | | | | |
| 885 | DRB1_ | 984 | SPALYY | 4 | 1266 | YYKIEP | 0.987 | Sequence | 0.427455 | 3.86 | <= |
| | 0102 | | KIEPRY | | | RYL | | | | | WB |
| | | | LDY | | | | | | | | |
| 885 | DRB1_ | 984 | SPALYY | 4 | 1266 | YYKIEP | 0.993 | Sequence | 0.799843 | 0.31 | <= |
| | 0103 | | KIEPRY | | | RYL | | | | | SB |
| | | | LDY | | | | | | | | |
| 885 | DRB1_ | 984 | SPALYY | 5 | 1267 | YKIEPR | 0.867 | Sequence | 0.139719 | 3.55 | <= |
| | 0305 | | KIEPRY | | | YLD | | | | | WB |
| | | | LDY | | | | | | | | |
| 885 | DRB1_ | 984 | SPALYY | 4 | 1266 | YYKIEP | 1 | Sequence | 0.604688 | 1.14 | <= |
| | 0701 | | KIEPRY | | | RYL | | | | | WB |
| | | | LDY | | | | | | | | |
| 885 | DRB1_ | 984 | SPALYY | 5 | 1267 | YKIEPR | 0.967 | Sequence | 0.869028 | 0.3 | <= |
| | 0801 | | KIEPRY | | | YLD | | | | | SB |
| | | | LDY | | | | | | | | |
| 885 | DRB1_ | 984 | SPALYY | 4 | 1266 | YYKIEP | 0.98 | Sequence | 0.558 | 1.61 | <= |
| | 0901 | | KIEPRY | | | RYL | | | | | WB |
| | | | LDY | | | | | | | | |
| 885 | DRB1_ | 984 | SPALYY | 4 | 1266 | YYKIEP | 0.98 | Sequence | 0.517413 | 2.79 | <= |
| | 1001 | | KIEPRY | | | RYL | | | | | WB |
| | | | LDY | | | | | | | | |
| 885 | DRB1_ | 984 | SPALYY | 5 | 1267 | YKIEPR | 0.973 | Sequence | 0.654392 | 1.8 | <= |
| | 1101 | | KIEPRY | | | YLD | | | | | WB |
| | | | LDY | | | | | | | | |
| 885 | DRB1_ | 984 | SPALYY | 3 | 1268 | LYYKIE | 0.713 | Sequence | 0.334476 | 3.43 | <= |
| | 1201 | | KIEPRY | | | PRY | | | | | WB |
| | | | LDY | | | | | | | | |
| 885 | DRB1_ | 984 | SPALYY | 3 | 1268 | LYYKIE | 0.787 | Sequence | 0.511546 | 3.48 | <= |
| | 1301 | | KIEPRY | | | PRY | | | | | WB |
| | | | LDY | | | | | | | | |
| 885 | DRB1_ | 984 | SPALYY | 5 | 1267 | YKIEPR | 0.553 | Sequence | 0.330651 | 4.36 | <= |
| | 1401 | | KIEPRY | | | YLD | | | | | WB |
| | | | LDY | | | | | | | | |
| 885 | DRB1_ | 984 | SPALYY | 4 | 1266 | YYKIEP | 0.96 | Sequence | 0.485374 | 2.67 | <= |
| | 1601 | | KIEPRY | | | RYL | | | | | WB |
| | | | LDY | | | | | | | | |
| 885 | DRB1_ | 984 | SPALYY | 5 | 1267 | YKIEPR | 0.553 | Sequence | 0.330651 | 4.36 | <= |
| | 1454 | | KIEPRY | | | YLD | | | | | WB |
| | | | LDY | | | | | | | | |
| 885 | DRB1_ | 984 | SPALYY | 5 | 1267 | YKIEPR | 0.747 | Sequence | 0.229834 | 2.25 | <= |
| | 1402 | | KIEPRY | | | YLD | | | | | WB |
| | | | LDY | | | | | | | | |
| 885 | DRB1_ | 984 | SPALYY | 5 | 1267 | YKIEPR | 0.993 | Sequence | 0.95436 | 0.04 | <= |
| | 1303 | | KIEPRY | | | YLD | | | | | SB |
| | | | LDY | | | | | | | | |
| 885 | DRB1_ | 984 | SPALYY | 5 | 1267 | YKIEPR | 0.46 | Sequence | 0.394144 | 2.39 | <= |
| | 1302 | | KIEPRY | | | YLD | | | | | WB |
| | | | LDY | | | | | | | | |
| 885 | DRB1_ | 984 | SPALYY | 5 | 1267 | YKIEPR | 0.86 | Sequence | 0.690316 | 0.22 | <= |
| | 0803 | | KIEPRY | | | YLD | | | | | SB |
| | | | LDY | | | | | | | | |
| 886 | DRB1_ | 985 | PALYYK | 3 | 1266 | YYKIEP | 1 | Sequence | 0.715566 | 1.27 | <= |
| | 0101 | | IEPRYL | | | RYL | | | | | WB |
| | | | DYF | | | | | | | | |
| 886 | DRB1_ | 985 | PALYYK | 3 | 1266 | YYKIEP | 0.987 | Sequence | 0.42909 | 3.84 | <= |
| | 0102 | | IEPRYL | | | RYL | | | | | WB |
| | | | DYF | | | | | | | | |
| 886 | DRB1_ | 985 | PALYYK | 3 | 1266 | YYKIEP | 0.993 | Sequence | 0.793886 | 0.33 | <= |
| | 0103 | | IEPRYL | | | RYL | | | | | SB |
| | | | DYF | | | | | | | | |
| 886 | DRB1_ | 985 | PALYYK | 4 | 1267 | YKIEPR | 0.933 | Sequence | 0.170393 | 2.91 | <= |
| | 0305 | | IEPRYL | | | YLD | | | | | WB |
| | | | DYF | | | | | | | | |
| 886 | DRB1_ | 985 | PALYYK | 3 | 1266 | YYKIEP | 1 | Sequence | 0.55721 | 1.41 | <= |
| | 0701 | | IEPRYL | | | RYL | | | | | WB |
| | | | DYF | | | | | | | | |
| 886 | DRB1_ | 985 | PALYYK | 4 | 1267 | YKIEPR | 0.993 | Sequence | 0.877682 | 0.27 | <= |
| | 0801 | | IEPRYL | | | YLD | | | | | SB |
| | | | DYF | | | | | | | | |
| 886 | DRB1_ | 985 | PALYYK | 3 | 1266 | YYKIEP | 0.98 | Sequence | 0.534722 | 1.84 | <= |
| | 0901 | | IEPRYL | | | RYL | | | | | WB |
| | | | DYF | | | | | | | | |
| 886 | DRB1_ | 985 | PALYYK | 3 | 1266 | YYKIEP | 0.96 | Sequence | 0.502876 | 2.96 | <= |
| | 1001 | | IEPRYL | | | RYL | | | | | WB |
| | | | DYF | | | | | | | | |
| 886 | DRB1_ | 985 | PALYYK | 4 | 1267 | YKIEPR | 0.987 | Sequence | 0.734491 | 1.33 | <= |
| | 1101 | | IEPRYL | | | YLD | | | | | WB |
| | | | DYF | | | | | | | | |
| 886 | DRB1_ | 985 | PALYYK | 3 | 1266 | YYKIEP | 0.973 | Sequence | 0.460718 | 2.96 | <= |
| | 1601 | | IEPRYL | | | RYL | | | | | WB |
| | | | DYF | | | | | | | | |
| 886 | DRB1_ | 985 | PALYYK | 4 | 1267 | YKIEPR | 0.84 | Sequence | 0.270963 | 1.47 | <= |
| | 1402 | | IEPRYL | | | YLD | | | | | WB |
| | | | DYF | | | | | | | | |
| 886 | DRB1_ | 985 | PALYYK | 4 | 1267 | YKIEPR | 1 | Sequence | 0.956938 | 0.03 | <= |
| | 1303 | | IEPRYL | | | YLD | | | | | SB |
| | | | DYF | | | | | | | | |
| 886 | DRB1_ | 985 | PAL YYKIEP | 4 | 1267 | YKIEPR | 0.667 | Sequence | 0.398901 | 2.3 | <= |
| | 1302 | | RYLDYF | | | YLD | | | | | WB |
| 886 | DRB1_ | 985 | PALYYK | 4 | 1267 | YKIEPR | 0.927 | Sequence | 0.715495 | 0.17 | <= |
| | 0803 | | IEPRYL | | | YLD | | | | | SB |
| | | | DYF | | | | | | | | |
| 887 | DRB1_ | 986 | ALYYKI | 2 | 1266 | YYKIEP | 0.98 | Sequence | 0.339247 | 4.03 | <= |
| | 0101 | | EPRYLD | | | RYL | | | | | WB |
| | | | YFK | | | | | | | | |
| 887 | DRB1_ | 986 | ALYYKI | 2 | 1266 | YYKIEP | 0.987 | Sequence | 0.479207 | 1.76 | <= |
| | 0103 | | EPRYLD | | | RYL | | | | | WB |
| | | | YFK | | | | | | | | |
| 887 | DRB1_ | 986 | ALYYKI | 3 | 1267 | YKIEPR | 0.967 | Sequence | 0.243405 | 1.98 | <= |
| | 0305 | | EPRYLD | | | YLD | | | | | WB |
| | | | YFK | | | | | | | | |
| 887 | DRB1_ | 986 | ALYYKI | 2 | 1266 | YYKIEP | 0.967 | Sequence | 0.239491 | 4.98 | <= |
| | 0701 | | EPRYLD | | | RYL | | | | | WB |
| | | | YFK | | | | | | | | |
| 887 | DRB1_ | 986 | ALYYKI | 3 | 1267 | YKIEPR | 1 | Sequence | 0.908993 | 0.16 | <= |
| | 0801 | | EPRYLD | | | YLD | | | | | SB |
| | | | YFK | | | | | | | | |
| 887 | DRB1_ | 986 | ALYYKI | 3 | 1267 | YKIEPR | 1 | Sequence | 0.835472 | 0.76 | <= |
| | 1101 | | EPRYLD | | | YLD | | | | | SB |
| | | | YFK | | | | | | | | |
| 887 | DRB1_ | 986 | ALYYKI | 3 | 1267 | YKIEPR | 0.98 | Sequence | 0.465704 | 3.87 | <= |
| | 1104 | | EPRYLD | | | YLD | | | | | WB |
| | | | YFK | | | | | | | | |
| 887 | DRB1_ | 986 | ALYYKI | 3 | 1267 | YKIEPR | 0.933 | Sequence | 0.346972 | 3.97 | <= |
| | 1401 | | EPRYLD | | | YLD | | | | | WB |
| | | | YFK | | | | | | | | |
| 887 | DRB1_ | 986 | ALYYKI | 3 | 1267 | YKIEPR | 0.933 | Sequence | 0.346972 | 3.97 | <= |
| | 1454 | | EPRYLD | | | YLD | | | | | WB |
| | | | YFK | | | | | | | | |
| 887 | DRB1_ | 986 | ALYYKI | 3 | 1267 | YKIEPR | 0.967 | Sequence | 0.336883 | 0.78 | <= |
| | 1402 | | EPRYLD | | | YLD | | | | | SB |
| | | | YFK | | | | | | | | |
| 887 | DRB1_ | 986 | ALYYKI | 3 | 1267 | YKIEPR | 1 | Sequence | 0.966706 | 0.01 | <= |
| | 1303 | | EPRYLD | | | YLD | | | | | SB |
| | | | YFK | | | | | | | | |
| 887 | DRB1_ | 986 | ALYYKI | 3 | 1267 | YKIEPR | 0.913 | Sequence | 0.462046 | 1.42 | <= |
| | 1302 | | EPRYLD | | | YLD | | | | | WB |
| | | | YFK | | | | | | | | |
| 887 | DRB1_ | 986 | ALYYKI | 3 | 1267 | YKIEPR | 0.98 | Sequence | 0.764743 | 0.1 | <= |
| | 0803 | | EPRYLD | | | YLD | | | | | SB |
| | | | YFK | | | | | | | | |
| 888 | DRB1_ | 987 | LYYKIE | 2 | 1267 | YKIEPR | 0.96 | Sequence | 0.118948 | 4.13 | <= |
| | 0305 | | PRYLDY | | | YLD | | | | | WB |
| | | | FKF | | | | | | | | |
| 888 | DRB1_ | 987 | LYYKIE | 2 | 1267 | YKIEPR | 1 | Sequence | 0.805681 | 0.59 | <= |
| | 0801 | | PRYLDY | | | YLD | | | | | SB |
| | | | FKF | | | | | | | | |
| 888 | DRB1_ | 987 | LYYKIE | 2 | 1267 | YKIEPR | 1 | Sequence | 0.625977 | 2 | <= |
| | 1101 | | PRYLDY | | | YLD | | | | | WB |
| | | | FKF | | | | | | | | |
| 888 | DRB1_ | 987 | LYYKIE | 2 | 1267 | YKIEPR | 0.98 | Sequence | 0.185711 | 3.61 | <= |
| | 1402 | | PRYLDY | | | YLD | | | | | WB |
| | | | FKF | | | | | | | | |
| 888 | DRB1_ | 987 | LYYKIE | 2 | 1267 | YKIEPR | 1 | Sequence | 0.934732 | 0.08 | <= |
| | 1303 | | PRYLDY | | | YLD | | | | | SB |
| | | | FKF | | | | | | | | |
| 888 | DRB1_ | 987 | LYYKIE | 2 | 1267 | YKIEPR | 0.98 | Sequence | 0.590242 | 0.55 | <= |
| | 0803 | | PRYLDY | | | YLD | | | | | SB |
| | | | FKF | | | | | | | | |
| 889 | DRB1_ | 988 | YYKIEP | 1 | 1267 | YKIEPR | 0.96 | Sequence | 0.517623 | 3.29 | <= |
| | 0801 | | RYLDYF | | | YLD | | | | | WB |
| | | | KFS | | | | | | | | |
| 889 | DRB1_ | 988 | YYKIEP | 1 | 1267 | YKIEPR | 1 | Sequence | 0.75804 | 0.69 | <= |
| | 1303 | | RYLDYF | | | YLD | | | | | SB |
| | | | KFS | | | | | | | | |
| 889 | DRB1_ | 988 | YYKIEP | 1 | 1267 | YKIEPR | 0.907 | Sequence | 0.300249 | 4.47 | <= |
| | 0803 | | RYLDYF | | | YLD | | | | | WB |
| | | | KFS | | | | | | | | |
| 900 | DRB1_ | 989 | FKFSFL | 4 | 1269 | FLVAYD | 0.967 | Sequence | 0.324439 | 4.21 | <= |
| | 0101 | | VAYDPL | | | PLI | | | | | WB |
| | | | INR | | | | | | | | |
| 900 | DRB1_ | 989 | FKFSFL | 6 | 1270 | VAYDPL | 0.993 | Sequence | 0.293882 | 3.87 | <= |
| | 0301 | | VAYDPL | | | INR | | | | | WB |
| | | | INR | | | | | | | | |
| 900 | DRB1_ | 989 | FKFSFL | 4 | 1269 | FLVAYD | 1 | Sequence | 0.367037 | 3.45 | <= |
| | 0401 | | VAYDPL | | | PLI | | | | | WB |
| | | | INR | | | | | | | | |
| 900 | DRB1_ | 989 | FKFSFL | 4 | 1269 | FLVAYD | 1 | Sequence | 0.374793 | 2.95 | <= |
| | 0405 | | VAYDPL | | | PLI | | | | | WB |
| | | | INR | | | | | | | | |
| 900 | DRB1_ | 989 | FKFSFL | 4 | 1269 | FLVAYD | 0.973 | Sequence | 0.251734 | 4.73 | <= |
| | 0701 | | VAYDPL | | | PLI | | | | | WB |
| | | | INR | | | | | | | | |
| 900 | DRB1_ | 989 | FKFSFL | 4 | 1269 | FLVAYD | 0.96 | Sequence | 0.475824 | 2.56 | <= |
| | 0901 | | VAYDPL | | | PLI | | | | | WB |
| | | | INR | | | | | | | | |
| 900 | DRB1_ | 989 | FKFSFL | 4 | 1269 | FLVAYD | 0.98 | Sequence | 0.602731 | 1.59 | <= |
| | 1601 | | VAYDPL | | | PLI | | | | | WB |
| | | | INR | | | | | | | | |
| 900 | DRB1_ | 989 | FKFSFL | 4 | 1269 | FLVAYD | 1 | Sequence | 0.45057 | 3.07 | <= |
| | 0408 | | VAYDPL | | | PLI | | | | | WB |
| | | | INR | | | | | | | | |
| 901 | DRB1_ | 990 | KFSFLV | 3 | 1269 | FLVAYD | 0.993 | Sequence | 0.358039 | 3.83 | <= |
| | 0101 | | AYDPLI | | | PLI | | | | | WB |
| | | | NRV | | | | | | | | |
| 901 | DRB1_ | 990 | KFSFLV | 5 | 1270 | VAYDPL | 1 | Sequence | 0.646548 | 1.24 | <= |
| | 0301 | | AYDPLI | | | INR | | | | | WB |
| | | | NRV | | | | | | | | |
| 901 | DRB1_ | 990 | KFSFLV | 5 | 1270 | VAYDPL | 0.82 | Sequence | 0.156182 | 3.18 | <= |
| | 0305 | | AYDPLI | | | INR | | | | | WB |
| | | | NRV | | | | | | | | |
| 901 | DRB1_ | 990 | KFSFLV | 3 | 1269 | FLVAYD | 1 | Sequence | 0.364402 | 3.48 | <= |
| | 0401 | | AYDPLI | | | PLI | | | | | WB |
| | | | NRV | | | | | | | | |
| 901 | DRB1_ | 990 | KFSFLV | 3 | 1269 | FLVAYD | 0.993 | Sequence | 0.359131 | 3.13 | <= |
| | 0405 | | AYDPLI | | | PLI | | | | | WB |
| | | | NRV | | | | | | | | |
| 901 | DRB1_ | 990 | KFSFLV | 3 | 1269 | FLVAYD | 0.973 | Sequence | 0.251584 | 4.73 | <= |
| | 0701 | | AYDPLI | | | PLI | | | | | WB |
| | | | NRV | | | | | | | | |
| 901 | DRB1_ | 990 | KFSFLV | 3 | 1269 | FLVAYD | 0.967 | Sequence | 0.477139 | 2.54 | <= |
| | 0901 | | AYDPLI | | | PLI | | | | | WB |
| | | | NRV | | | | | | | | |
| 901 | DRB1_ | 990 | KFSFLV | 3 | 1269 | FLVAYD | 0.973 | Sequence | 0.61946 | 1.46 | <= |
| | 1601 | | AYDPLI | | | PLI | | | | | WB |
| | | | NRV | | | | | | | | |
| 901 | DRB1_ | 990 | KFSFLV | 3 | 1269 | FLVAYD | 1 | Sequence | 0.446516 | 3.12 | <= |
| | 0408 | | AYDPLI | | | PLI | | | | | WB |
| | | | NRV | | | | | | | | |
| 902 | DRB1_ | 991 | FSFLVA | 4 | 1270 | VAYDPL | 1 | Sequence | 0.769309 | 0.7 | <= |
| | 0301 | | YDPLIN | | | INR | | | | | SB |
| | | | RVS | | | | | | | | |
| 902 | DRB1_ | 991 | FSFLVA | 4 | 1270 | VAYDPL | 0.927 | Sequence | 0.211779 | 2.33 | <= |
| | 0305 | | YDPLIN | | | INR | | | | | WB |
| | | | RVS | | | | | | | | |
| 903 | DRB1_ | 992 | SFLVAY | 3 | 1270 | VAYDPL | 1 | Sequence | 0.819567 | 0.5 | <= |
| | 0301 | | DPLINR | | | INR | | | | | SB |
| | | | VSE | | | | | | | | |
| 903 | DRB1_ | 992 | SFLVAY | 3 | 1270 | VAYDPL | 0.98 | Sequence | 0.261071 | 1.8 | <= |
| | 0305 | | DPLINR | | | INR | | | | | WB |
| | | | VSE | | | | | | | | |
| 903 | DRB1_ | 992 | SFLVAY | 5 | 1271 | YDPLIN | 0.92 | Sequence | 0.328969 | 4.82 | <= |
| | 0408 | | DPLINR | | | RVS | | | | | WB |
| | | | VSE | | | | | | | | |
| 904 | DRB1_ | 993 | FLVAYD | 2 | 1270 | VAYDPL | 1 | Sequence | 0.610679 | 1.41 | <= |
| | 0301 | | PLINRV | | | INR | | | | | WB |
| | | | SEL | | | | | | | | |
| 904 | DRB1_ | 993 | FLVAYD | 2 | 1270 | VAYDPL | 0.953 | Sequence | 0.124036 | 3.97 | <= |
| | 0305 | | PLINRV | | | INR | | | | | WB |
| | | | SEL | | | | | | | | |
| 904 | DRB1_ | 993 | FLVAYD | 4 | 1271 | YDPLIN | 1 | Sequence | 0.346282 | 4.52 | <= |
| | 0408 | | PLINRV | | | RVS | | | | | WB |
| | | | SEL | | | | | | | | |
| 905 | DRB1_ | 994 | LVAYDP | 3 | 1271 | YDPLIN | 0.973 | Sequence | 0.258985 | 4.52 | <= |
| | 0103 | | LINRVS | | | RVS | | | | | WB |
| | | | ELS | | | | | | | | |
| 905 | DRB1_ | 994 | LVAYDP | 1 | 1270 | VAYDPL | 1 | Sequence | 0.285961 | 3.97 | <= |
| | 0301 | | LINRVS | | | INR | | | | | WB |
| | | | ELS | | | | | | | | |
| 905 | DRB1_ | 994 | LVAYDP | 3 | 1271 | YDPLIN | 1 | Sequence | 0.393749 | 3.78 | <= |
| | 0408 | | LINRVS | | | RVS | | | | | WB |
| | | | ELS | | | | | | | | |
| 907 | DRB1_ | 995 | AYDPLI | 4 | 1272 | LINRVS | 0.953 | Sequence | 0.274747 | 4.47 | <= |
| | 0402 | | NRVSEL | | | ELS | | | | | WB |
| | | | SFK | | | | | | | | |
| 907 | DRB1_ | 995 | AYDPLI | 5 | 1273 | INRVSE | 0.98 | Sequence | 0.428678 | 2.01 | <= |
| | 1201 | | NRVSEL | | | LSF | | | | | WB |
| | | | SFK | | | | | | | | |
| 908 | DRB1_ | 996 | YDPLIN | 4 | 1273 | INRVSE | 0.973 | Sequence | 0.457327 | 1.71 | <= |
| | 1201 | | RVSELS | | | LSF | | | | | WB |
| | | | FKL | | | | | | | | |
| 909 | DRB1_ | 997 | DPLINR | 3 | 1273 | INRVSE | 0.993 | Sequence | 0.551334 | 1.03 | <= |
| | 1201 | | VSELSF | | | LSF | | | | | WB |
| | | | KLN | | | | | | | | |
| 910 | DRB1_ | 998 | PLINRV | 2 | 1273 | INRVSE | 0.967 | Sequence | 0.310282 | 3.96 | <= |
| | 1201 | | SELSFK | | | LSF | | | | | WB |
| | | | LNV | | | | | | | | |
| 922 | DRB1_ | 999 | LNVFDF | 5 | 1274 | FQFLFA | 1 | Sequence | 0.274091 | 4.43 | <= |
| | 0405 | | QFLFAM | | | MKD | | | | | WB |
| | | | KDE | | | | | | | | |
| 922 | DRB1_ | 999 | LNVFDF | 5 | 1274 | FQFLFA | 0.98 | Sequence | 0.463527 | 3.52 | <= |
| | 1001 | | QFLFAM | | | MKD | | | | | WB |
| | | | KDE | | | | | | | | |
| 923 | DRB1_ | 1000 | NVFDFQ | 4 | 1274 | FQFLFA | 0.993 | Sequence | 0.305403 | 3.89 | <= |
| | 0405 | | FLFAMK | | | MKD | | | | | WB |
| | | | DEF | | | | | | | | |
| 923 | DRB1_ | 1000 | NVFDFQ | 4 | 1274 | FQFLFA | 1 | Sequence | 0.510395 | 2.87 | <= |
| | 1001 | | FLFAMK | | | MKD | | | | | WB |
| | | | DEF | | | | | | | | |
| 924 | DRB1_ | 1001 | VFDFQF | 3 | 1274 | FQFLFA | 1 | Sequence | 0.27607 | 4.85 | <= |
| | 0101 | | LFAMKD | | | MKD | | | | | WB |
| | | | EFE | | | | | | | | |
| 924 | DRB1_ | 1001 | VFDFQF | 3 | 1274 | FQFLFA | 0.9 | Sequence | 0.428955 | 2.41 | <= |
| | 0405 | | LFAMKD | | | MKD | | | | | WB |
| | | | EFE | | | | | | | | |
| 924 | DRB1_ | 1001 | VFDFQF | 3 | 1274 | FQFLFA | 1 | Sequence | 0.621245 | 1.71 | <= |
| | 1001 | | LFAMKD | | | MKD | | | | | WB |
| | | | EFE | | | | | | | | |
| 925 | DRB1_ | 1002 | FDFQFL | 5 | 1275 | LFAMKD | 0.86 | Sequence | 0.486759 | 3.13 | <= |
| | 0403 | | FAMKDE | | | EFE | | | | | WB |
| | | | FEY | | | | | | | | |
| 925 | DRB1_ | 1002 | FDFQFL | 5 | 1275 | LFAMKD | 0.747 | Sequence | 0.486413 | 1.95 | <= |
| | 0405 | | FAMKDE | | | EFE | | | | | WB |
| | | | FEY | | | | | | | | |
| 925 | DRB1_ | 1002 | FDFQFL | 2 | 1274 | FQFLFA | 0.987 | Sequence | 0.448017 | 3.78 | <= |
| | 1001 | | FAMKDE | | | MKD | | | | | WB |
| | | | FEY | | | | | | | | |
| 926 | DRB1_ | 1003 | DFQFLF | 4 | 1275 | LFAMKD | 0.987 | Sequence | 0.680627 | 1.17 | <= |
| | 0403 | | AMKDEF | | | EFE | | | | | WB |
| | | | EYN | | | | | | | | |
| 926 | DRB1_ | 1003 | DFQFLF | 4 | 1275 | LFAMKD | 0.993 | Sequence | 0.366935 | 4.32 | <= |
| | 0404 | | AMKDEF | | | EFE | | | | | WB |
| | | | EYN | | | | | | | | |
| 926 | DRB1_ | 1003 | DFQFLF | 4 | 1275 | LFAMKD | 0.98 | Sequence | 0.64421 | 1.08 | <= |
| | 0405 | | AMKDEF | | | EFE | | | | | WB |
| | | | EYN | | | | | | | | |
| 926 | DRB1_ | 1003 | DFQFLF | 3 | 1276 | FLFAMK | 0.893 | Sequence | 0.333476 | 4.29 | <= |
| | 1401 | | AMKDEF | | | DEF | | | | | WB |
| | | | EYN | | | | | | | | |
| 926 | DRB1_ | 1003 | DFQFLF | 3 | 1276 | FLFAMK | 0.893 | Sequence | 0.333476 | 4.29 | <= |
| | 1454 | | AMKDEF | | | DEF | | | | | WB |
| | | | EYN | | | | | | | | |
| 927 | DRB1_ | 1004 | FQFLFA | 3 | 1275 | LFAMKD | 1 | Sequence | 0.693216 | 1.09 | <= |
| | 0403 | | MKDEFE | | | EFE | | | | | WB |
| | | | YNY | | | | | | | | |
| 927 | DRB1_ | 1004 | FQFLFA | 3 | 1275 | LFAMKD | 0.993 | Sequence | 0.417478 | 3.62 | <= |
| | 0404 | | MKDEFE | | | EFE | | | | | WB |
| | | | YNY | | | | | | | | |
| 927 | DRB1_ | 1004 | FQFLFA | 3 | 1275 | LFAMKD | 1 | Sequence | 0.651081 | 1.05 | <= |
| | 0405 | | MKDEFE | | | EFE | | | | | WB |
| | | | YNY | | | | | | | | |
| 928 | DRB1_ | 1005 | QFLFAM | 2 | 1275 | LFAMKD | 1 | Sequence | 0.537304 | 2.47 | <= |
| | 0403 | | KDEFEY | | | EFE | | | | | WB |
| | | | NYD | | | | | | | | |
| 928 | DRB1_ | 1005 | QFLFAM | 2 | 1275 | LFAMKD | 1 | Sequence | 0.467705 | 2.09 | <= |
| | 0405 | | KDEFEY | | | EFE | | | | | WB |
| | | | NYD | | | | | | | | |
| 933 | DRB1_ | 1006 | MKDEFE | 4 | 1277 | FEYNYD | 0.827 | Sequence | 0.172456 | 2.88 | <= |
| | 0305 | | YNYDPL | | | PLK | | | | | WB |
| | | | KGD | | | | | | | | |
| 933 | DRB1_ | 1006 | MKDEFE | 4 | 1277 | FEYNYD | 0.973 | Sequence | 0.285239 | 4.82 | <= |
| | 0401 | | YNYDPL | | | PLK | | | | | WB |
| | | | KGD | | | | | | | | |
| 933 | DRB1_ | 1006 | MKDEFE | 4 | 1277 | FEYNYD | 0.927 | Sequence | 0.200938 | 3.05 | <= |
| | 1402 | | YNYDPL | | | PLK | | | | | WB |
| | | | KGD | | | | | | | | |
| 934 | DRB1_ | 1007 | KDEFEY | 3 | 1277 | FEYNYD | 0.633 | Sequence | 0.199042 | 2.48 | <= |
| | 0305 | | NYDPLK | | | PLK | | | | | WB |
| | | | GDF | | | | | | | | |
| 934 | DRB1_ | 1007 | KDEFEY | 3 | 1277 | FEYNYD | 0.84 | Sequence | 0.289904 | 4.72 | <= |
| | 0401 | | NYDPLK | | | PLK | | | | | WB |
| | | | GDF | | | | | | | | |
| 934 | DRB1_ | 1007 | KDEFEY | 3 | 1277 | FEYNYD | 0.84 | Sequence | 0.217644 | 2.57 | <= |
| | 1402 | | NYDPLK | | | PLK | | | | | WB |
| | | | GDF | | | | | | | | |
| 934 | DRB1_ | 1007 | KDEFEY | 5 | 1278 | YNYDPL | 0.76 | Sequence | 0.420759 | 3.26 | <= |
| | 1303 | | NYDPLK | | | KGD | | | | | WB |
| | | | GDF | | | | | | | | |
| 934 | DRB1_ | 1007 | KDEFEY | 3 | 1277 | FEYNYD | 0.907 | Sequence | 0.300157 | 4.88 | <= |
| | 1302 | | NYDPLK | | | PLK | | | | | WB |
| | | | GDF | | | | | | | | |
| 935 | DRB1_ | 1008 | DEFEYN | 4 | 1278 | YNYDPL | 0.667 | Sequence | 0.160477 | 3.09 | <= |
| | 0305 | | YDPLKG | | | KGD | | | | | WB |
| | | | DFS | | | | | | | | |
| 935 | DRB1_ | 1008 | DEFEYN | 4 | 1278 | YNYDPL | 0.947 | Sequence | 0.524326 | 2.16 | <= |
| | 1303 | | YDPLKG | | | KGD | | | | | WB |
| | | | DFS | | | | | | | | |
| 936 | DRB1_ | 1009 | EFEYNY | 5 | 1279 | YDPLKG | 0.993 | Sequence | 0.412952 | 3.29 | <= |
| | 0101 | | DPLKGD | | | DFS | | | | | WB |
| | | | FSK | | | | | | | | |
| 936 | DRB1_ | 1009 | EFEYNY | 3 | 1278 | YNYDPL | 0.953 | Sequence | 0.177471 | 2.8 | <= |
| | 0305 | | DPLKGD | | | KGD | | | | | WB |
| | | | FSK | | | | | | | | |
| 936 | DRB1_ | 1009 | EFEYNY | 3 | 1278 | YNYDPL | 0.793 | Sequence | 0.259511 | 4.72 | <= |
| | 0405 | | DPLKGD | | | KGD | | | | | WB |
| | | | FSK | | | | | | | | |
| 936 | DRB1_ | 1009 | EFEYNY | 5 | 1279 | YDPLKG | 0.953 | Sequence | 0.52626 | 2.68 | <= |
| | 1001 | | DPLKGD | | | DFS | | | | | WB |
| | | | FSK | | | | | | | | |
| 936 | DRB1_ | 1009 | EFEYNY | 3 | 1278 | YNYDPL | 0.987 | Sequence | 0.635007 | 1.33 | <= |
| | 1303 | | DPLKGD | | | KGD | | | | | WB |
| | | | FSK | | | | | | | | |
| 936 | DRB1_ | 1009 | EFEYNY | 3 | 1278 | YNYDPL | 0.88 | Sequence | 0.285332 | 4.96 | <= |
| | 0803 | | DPLKGD | | | KGD | | | | | WB |
| | | | FSK | | | | | | | | |
| 937 | DRB1_ | 1010 | FEYNYD | 4 | 1279 | YDPLKG | 1 | Sequence | 0.560414 | 2.12 | <= |
| | 0101 | | PLKGDF | | | DFS | | | | | WB |
| | | | SKI | | | | | | | | |
| 937 | DRB1_ | 1010 | FEYNYD | 4 | 1279 | YDPLKG | 0.987 | Sequence | 0.599202 | 1.92 | <= |
| | 1001 | | PLKGDF | | | DFS | | | | | WB |
| | | | SKI | | | | | | | | |
| 937 | DRB1_ | 1010 | FEYNYD | 2 | 1278 | YNYDPL | 0.967 | Sequence | 0.387503 | 3.67 | <= |
| | 1303 | | PLKGDF | | | KGD | | | | | WB |
| | | | SKI | | | | | | | | |
| 938 | DRB1_ | 1011 | EYNYDP | 3 | 1279 | YDPLKG | 1 | Sequence | 0.705123 | 1.33 | <= |
| | 0101 | | LKGDFS | | | DFS | | | | | WB |
| | | | KIG | | | | | | | | |
| 938 | DRB1_ | 1011 | EYNYDP | 3 | 1279 | YDPLKG | 1 | Sequence | 0.464316 | 3.45 | <= |
| | 0102 | | LKGDFS | | | DFS | | | | | WB |
| | | | KIG | | | | | | | | |
| 938 | DRB1_ | 1011 | EYNYDP | 3 | 1279 | YDPLKG | 0.98 | Sequence | 0.312996 | 3.58 | <= |
| | 0103 | | LKGDFS | | | DFS | | | | | WB |
| | | | KIG | | | | | | | | |
| 938 | DRB1_ | 1011 | EYNYDP | 3 | 1279 | YDPLKG | 1 | Sequence | 0.679572 | 1.26 | <= |
| | 1001 | | LKGDFS | | | DFS | | | | | WB |
| | | | KIG | | | | | | | | |
| 939 | DRB1_ | 1012 | YNYDPL | 2 | 1279 | YDPLKG | 1 | Sequence | 0.323518 | 4.22 | <= |
| | 0101 | | KGDFSK | | | DFS | | | | | WB |
| | | | IGT | | | | | | | | |
| 939 | DRB1_ | 1012 | YNYDPL | 2 | 1279 | YDPLKG | 0.967 | Sequence | 0.410043 | 4.44 | <= |
| | 1001 | | KGDFSK | | | DFS | | | | | WB |
| | | | IGT | | | | | | | | |
| 953 | DRB1_ | 1013 | TTTKLV | 4 | 1280 | LVPYSL | 1 | Sequence | 0.448318 | 1.97 | <= |
| | 0402 | | PYSLDS | | | DSS | | | | | WB |
| | | | SYK | | | | | | | | |
| 954 | DRB1_ | 1014 | TTKLVP | 3 | 1280 | LVPYSL | 1 | Sequence | 0.566322 | 1.15 | <= |
| | 0402 | | YSLDSS | | | DSS | | | | | WB |
| | | | YKK | | | | | | | | |
| 954 | DRB1_ | 1014 | TTKLVP | 3 | 1280 | LVPYSL | 0.987 | Sequence | 0.213327 | 4.71 | <= |
| | 1501 | | YSLDSS | | | DSS | | | | | WB |
| | | | YKK | | | | | | | | |
| 955 | DRB1_ | 1015 | TKLVPY | 5 | 1281 | YSLDSS | 0.967 | Sequence | 0.252634 | 4.48 | <= |
| | 0301 | | SLDSSY | | | YKK | | | | | WB |
| | | | KKE | | | | | | | | |
| 955 | DRB1_ | 1015 | TKLVPY | 5 | 1281 | YSLDSS | 0.98 | Sequence | 0.225106 | 2.18 | <= |
| | 0305 | | SLDSSY | | | YKK | | | | | WB |
| | | | KKE | | | | | | | | |
| 955 | DRB1_ | 1015 | TKLVPY | 2 | 1280 | LVPYSL | 0.967 | Sequence | 0.379301 | 2.73 | <= |
| | 0402 | | SLDSSY | | | DSS | | | | | WB |
| | | | KKE | | | | | | | | |
| 956 | DRB1_ | 1016 | KLVPYS | 4 | 1281 | YSLDSS | 0.987 | Sequence | 0.281707 | 4.03 | <= |
| | 0301 | | LDSSYK | | | YKK | | | | | WB |
| | | | KEL | | | | | | | | |
| 956 | DRB1_ | 1016 | KLVPYS | 4 | 1281 | YSLDSS | 0.993 | Sequence | 0.250266 | 1.9 | <= |
| | 0305 | | LDSSYK | | | YKK | | | | | WB |
| | | | KEL | | | | | | | | |
| 957 | DRB1_ | 1017 | LVPYSL | 3 | 1281 | YSLDSS | 1 | Sequence | 0.302538 | 3.76 | <= |
| | 0301 | | DSSYKK | | | YKK | | | | | WB |
| | | | ELY | | | | | | | | |
| 957 | DRB1_ | 1017 | LVPYSL | 3 | 1281 | YSLDSS | 0.98 | Sequence | 0.266742 | 1.74 | <= |
| | 0305 | | DSSYKK | | | YKK | | | | | WB |
| | | | ELY | | | | | | | | |
| 958 | DRB1_ | 1018 | VPYSLD | 2 | 1281 | YSLDSS | 0.973 | Sequence | 0.168205 | 2.94 | <= |
| | 0305 | | SSYKKE | | | YKK | | | | | WB |
| | | | LYV | | | | | | | | |
| 967 | DRB1_ | 1019 | KKELYV | 5 | 1282 | VLTFFD | 0.833 | Sequence | 0.284539 | 3.49 | <= |
| | 1501 | | LTFFDN | | | NKL | | | | | WB |
| | | | KLS | | | | | | | | |
| 967 | DRB1_ | 1019 | KKELYV | 5 | 1282 | VLTFFD | 0.773 | Sequence | 0.192341 | 4.31 | <= |
| | 1503 | | LTFFDN | | | NKL | | | | | WB |
| | | | KLS | | | | | | | | |
| 968 | DRB1_ | 1020 | KELYVL | 5 | 1283 | LTFFDN | 0.96 | Sequence | 0.359286 | 2.98 | <= |
| | 0402 | | TFFDNK | | | KLS | | | | | WB |
| | | | LSF | | | | | | | | |
| 968 | DRB1_ | 1020 | KELYVL | 5 | 1283 | LTFFDN | 0.56 | Sequence | 0.530291 | 1.52 | <= |
| | 1501 | | TFFDNK | | | KLS | | | | | WB |
| | | | LSF | | | | | | | | |
| 968 | DRB1_ | 1020 | KELYVL | 4 | 1282 | VLTFFD | 0.513 | Sequence | 0.320656 | 2.19 | <= |
| | 1503 | | TFFDNK | | | NKL | | | | | WB |
| | | | LSF | | | | | | | | |
| 969 | DRB1_ | 1021 | ELYVLT | 4 | 1283 | LTFFDN | 0.987 | Sequence | 0.516896 | 1.44 | <= |
| | 0402 | | FFDNKL | | | KLS | | | | | WB |
| | | | SFT | | | | | | | | |
| 969 | DRB1_ | 1021 | ELYVLT | 4 | 1283 | LTFFDN | 0.633 | Sequence | 0.668288 | 0.92 | <= |
| | 1501 | | FFDNKL | | | KLS | | | | | SB |
| | | | SFT | | | | | | | | |
| 969 | DRB1_ | 1021 | ELYVLT | 4 | 1283 | LTFFDN | 0.547 | Sequence | 0.415469 | 1.41 | <= |
| | 1503 | | FFDNKL | | | KLS | | | | | WB |
| | | | SFT | | | | | | | | |
| 970 | DRB1_ | 1022 | LYVLTF | 3 | 1283 | LTFFDN | 1 | Sequence | 0.402361 | 2.45 | <= |
| | 0402 | | FDNKLS | | | KLS | | | | | WB |
| | | | FTL | | | | | | | | |
| 970 | DRB1_ | 1022 | LYVLTF | 3 | 1283 | LTFFDN | 0.867 | Sequence | 0.526299 | 1.55 | <= |
| | 1501 | | FDNKLS | | | KLS | | | | | WB |
| | | | FTL | | | | | | | | |
| 970 | DRB1_ | 1022 | LYVLTF | 3 | 1283 | LTFFDN | 0.767 | Sequence | 0.281417 | 2.65 | <= |
| | 1503 | | FDNKLS | | | KLS | | | | | WB |
| | | | FTL | | | | | | | | |
| 971 | DRB1_ | 1023 | YVLTFF | 2 | 1283 | LTFFDN | 1 | Sequence | 0.277371 | 4.42 | <= |
| | 0402 | | DNKLSF | | | KLS | | | | | WB |
| | | | TLG | | | | | | | | |
| 971 | DRB1_ | 1023 | YVLTFF | 2 | 1283 | LTFFDN | 0.92 | Sequence | 0.33217 | 2.95 | <= |
| | 1501 | | DNKLSF | | | KLS | | | | | WB |
| | | | TLG | | | | | | | | |
| 971 | DRB1_ | 1023 | YVLTFF | 2 | 1283 | LTFFDN | 0.86 | Sequence | 0.175716 | 4.8 | <= |
| | 1503 | | DNKLSF | | | KLS | | | | | WB |
| | | | TLG | | | | | | | | |
| 974 | DRB1_ | 1024 | TFFDNK | 6 | 1284 | LSFTLG | 0.947 | Sequence | 0.27148 | 4.36 | <= |
| | 0701 | | LSFTLG | | | VDV | | | | | WB |
| | | | VDV | | | | | | | | |
| 975 | DRB1_ | 1025 | FFDNKL | 5 | 1284 | LSFTLG | 1 | Sequence | 0.618981 | 1.06 | <= |
| | 0701 | | SFTLGV | | | VDV | | | | | WB |
| | | | DVG | | | | | | | | |
| 975 | DRB1_ | 1025 | FFDNKL | 5 | 1284 | LSFTLG | 0.967 | Sequence | 0.38402 | 4.21 | <= |
| | 0901 | | SFTLGV | | | VDV | | | | | WB |
| | | | DVG | | | | | | | | |
| 976 | DRB1_ | 1026 | FDNKLS | 4 | 1284 | LSFTLG | 1 | Sequence | 0.619468 | 1.06 | <= |
| | 0701 | | FTLGVD | | | VDV | | | | | WB |
| | | | VGW | | | | | | | | |
| 976 | DRB1_ | 1026 | FDNKLS | 4 | 1284 | LSFTLG | 0.98 | Sequence | 0.395448 | 3.96 | <= |
| | 0901 | | FTLGVD | | | VDV | | | | | WB |
| | | | VGW | | | | | | | | |
| 977 | DRB1_ | 1027 | DNKLSF | 3 | 1284 | LSFTLG | 1 | Sequence | 0.349436 | 4.93 | <= |
| | 0102 | | TLGVDV | | | VDV | | | | | WB |
| | | | GWK | | | | | | | | |
| 977 | DRB1_ | 1027 | DNKLSF | 3 | 1284 | LSFTLG | 1 | Sequence | 0.747632 | 0.55 | <= |
| | 0701 | | TLGVDV | | | VDV | | | | | SB |
| | | | GWK | | | | | | | | |
| 977 | DRB1_ | 1027 | DNKLSF | 3 | 1284 | LSFTLG | 0.98 | Sequence | 0.519299 | 2.01 | <= |
| | 0901 | | TLGVDV | | | VDV | | | | | WB |
| | | | GWK | | | | | | | | |
| 978 | DRB1_ | 1028 | NKLSFT | 2 | 1284 | LSFTLG | 0.993 | Sequence | 0.416503 | 2.44 | <= |
| | 0701 | | LGVDVG | | | VDV | | | | | WB |
| | | | WKI | | | | | | | | |
| 989 | DRB1_ | 1029 | GWKINL | 5 | 1285 | LQKFTD | 0.947 | Sequence | 0.279891 | 4.36 | <= |
| | 0402 | | QKFTDN | | | NEL | | | | | WB |
| | | | ELR | | | | | | | | |
| 989 | DRB1_ | 1029 | GWKINL | 5 | 1285 | LQKFTD | 1 | Sequence | 0.742588 | 0.68 | <= |
| | 1501 | | QKFTDN | | | NEL | | | | | SB |
| | | | ELR | | | | | | | | |
| 989 | DRB1_ | 1029 | GWKINL | 5 | 1285 | LQKFTD | 1 | Sequence | 0.559637 | 0.72 | <= |
| | 1503 | | QKFTDN | | | NEL | | | | | SB |
| | | | ELR | | | | | | | | |
| 990 | DRB1_ | 1030 | WKINLQ | 4 | 1285 | LQKFTD | 1 | Sequence | 0.446032 | 1.99 | <= |
| | 0402 | | KFTDNE | | | NEL | | | | | WB |
| | | | LRS | | | | | | | | |
| 990 | DRB1_ | 1030 | WKINLQ | 4 | 1285 | LQKFTD | 1 | Sequence | 0.36503 | 4.35 | <= |
| | 0404 | | KFTDNE | | | NEL | | | | | WB |
| | | | LRS | | | | | | | | |
| 990 | DRB1_ | 1030 | WKINLQ | 4 | 1285 | LQKFTD | 1 | Sequence | 0.874292 | 0.32 | <= |
| | 1501 | | KFTDNE | | | NEL | | | | | SB |
| | | | LRS | | | | | | | | |
| 990 | DRB1_ | 1030 | WKINLQ | 4 | 1285 | LQKFTD | 1 | Sequence | 0.465189 | 2.91 | <= |
| | 1601 | | KFTDNE | | | NEL | | | | | WB |
| | | | LRS | | | | | | | | |
| 990 | DRB1_ | 1030 | WKINLQ | 4 | 1285 | LQKFTD | 1 | Sequence | 0.713012 | 0.3 | <= |
| | 1503 | | KFTDNE | | | NEL | | | | | SB |
| | | | LRS | | | | | | | | |
| 991 | DRB1_ | 1031 | KINLQK | 3 | 1285 | LQKFTD | 1 | Sequence | 0.524487 | 1.39 | <= |
| | 0402 | | FTDNEL | | | NEL | | | | | WB |
| | | | RSA | | | | | | | | |
| 991 | DRB1_ | 1031 | KINLQK | 3 | 1285 | LQKFTD | 1 | Sequence | 0.436713 | 3.39 | <= |
| | 0404 | | FTDNEL | | | NEL | | | | | WB |
| | | | RSA | | | | | | | | |
| 991 | DRB1_ | 1031 | KINLQK | 3 | 1285 | LOKFTD | 1 | Sequence | 0.912524 | 0.21 | <= |
| | 1501 | | FTDNEL | | | NEL | | | | | SB |
| | | | RSA | | | | | | | | |
| 991 | DRB1_ | 1031 | KINLQK | 3 | 1285 | LQKFTD | 1 | Sequence | 0.561647 | 1.9 | <= |
| | 1601 | | FTDNEL | | | NEL | | | | | WB |
| | | | RSA | | | | | | | | |
| 991 | DRB1_ | 1031 | KINLQK | 3 | 1285 | LQKFTD | 1 | Sequence | 0.779448 | 0.19 | <= |
| | 1503 | | FTDNEL | | | NEL | | | | | SB |
| | | | RSA | | | | | | | | |
| 992 | DRB1_ | 1032 | INLQKF | 2 | 1285 | LQKFTD | 1 | Sequence | 0.664379 | 0.94 | <= |
| | 1501 | | TDNELR | | | NEL | | | | | SB |
| | | | SAL | | | | | | | | |
| 992 | DRB1_ | 1032 | INLQKF | 2 | 1285 | LQKFTD | 1 | Sequence | 0.502654 | 0.94 | <= |
| | 1503 | | TDNELR | | | NEL | | | | | SB |
| | | | SAL | | | | | | | | |
| 997 | DRB1_ | 1033 | FTDNEL | 5 | 1286 | LRSALT | 1 | Sequence | 0.388426 | 4.34 | <= |
| | 0102 | | RSALTL | | | LKL | | | | | WB |
| | | | KLK | | | | | | | | |
| 997 | DRB1_ | 1033 | FTDNEL | 5 | 1286 | LRSALT | 1 | Sequence | 0.398541 | 2.61 | <= |
| | 0701 | | RSALTL | | | LKL | | | | | WB |
| | | | KLK | | | | | | | | |
| 997 | DRB1_ | 1033 | FTDNEL | 5 | 1286 | LRSALT | 1 | Sequence | 0.512878 | 2.09 | <= |
| | 0901 | | RSALTL | | | LKL | | | | | WB |
| | | | KLK | | | | | | | | |
| 997 | DRB1_ | 1033 | FTDNEL | 5 | 1286 | LRSALT | 0.993 | Sequence | 0.305258 | 4.99 | <= |
| | 1401 | | RSALTL | | | LKL | | | | | WB |
| | | | KLK | | | | | | | | |
| 997 | DRB1_ | 1033 | FTDNEL | 5 | 1286 | LRSALT | 0.993 | Sequence | 0.305258 | 4.99 | <= |
| | 1454 | | RSALTL | | | LKL | | | | | WB |
| | | | KLK | | | | | | | | |
| 998 | DRB1_ | 1034 | TDNELR | 4 | 1286 | LRSALT | 1 | Sequence | 0.283964 | 4.73 | <= |
| | 0101 | | SALTLK | | | LKL | | | | | WB |
| | | | LKY | | | | | | | | |
| 998 | DRB1_ | 1034 | TDNELR | 4 | 1286 | LRSALT | 1 | Sequence | 0.605295 | 2.13 | <= |
| | 0102 | | SALTLK | | | LKL | | | | | WB |
| | | | LKY | | | | | | | | |
| 998 | DRB1_ | 1034 | TDNELR | 4 | 1286 | LRSALT | 0.973 | Sequence | 0.334464 | 3.26 | <= |
| | 0103 | | SALTLK | | | LKL | | | | | WB |
| | | | LKY | | | | | | | | |
| 998 | DRB1_ | 1034 | TDNELR | 4 | 1286 | LRSALT | 1 | Sequence | 0.467251 | 2.02 | <= |
| | 0701 | | SALTLK | | | LKL | | | | | WB |
| | | | LKY | | | | | | | | |
| 998 | DRB1_ | 1034 | TDNELR | 4 | 1286 | LRSALT | 1 | Sequence | 0.579474 | 1.39 | <= |
| | 0901 | | SALTLK | | | LKL | | | | | WB |
| | | | LKY | | | | | | | | |
| 998 | DRB1_ | 1034 | TDNELR | 4 | 1286 | LRSALT | 1 | Sequence | 0.389369 | 3.13 | <= |
| | 1401 | | SALTLK | | | LKL | | | | | WB |
| | | | LKY | | | | | | | | |
| 998 | DRB1_ | 1034 | TDNELR | 4 | 1286 | LRSALT | 1 | Sequence | 0.389369 | 3.13 | <= |
| | 1454 | | SALTLK | | | LKL | | | | | WB |
| | | | LKY | | | | | | | | |
| 999 | DRB1_ | 1035 | DNELRS | 3 | 1286 | LRSALT | 1 | Sequence | 0.352464 | 3.89 | <= |
| | 0101 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.6725 | 1.65 | <= |
| | 0102 | | ALTLKL | | | LKL | | | | | WB |
| | | | KYT | | | | | | | | |
| 999 | DRB1_ | 1035 | DNELRS | 3 | 1286 | LRSALT | 0.993 | Sequence | 0.402043 | 2.44 | <= |
| | 0103 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.254491 | 4.94 | <= |
| | 0402 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.475707 | 1.95 | <= |
| | 0701 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.595273 | 1.26 | <= |
| | 0901 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.390357 | 4.82 | <= |
| | 1001 | | ALTLKL | | | LKL | | | | | WB |
| | | | KYT | | | | | | | | |
| 999 | DRB1_ | 1035 | DNELRS | 3 | 1286 | LRSALT | 0.967 | Sequence | 0.288115 | 4.5 | <= |
| | 1201 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.408351 | 2.82 | <= |
| | 1401 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.338887 | 4.67 | <= |
| | 1601 | | ALTLKL | | | LKL | | | | | WB |
| | | | KYT | | | | | | | | |
| 999 | DRB1_ | 1035 | DNELRS | 3 | 1286 | LRSALT | 1 | Sequence | 0.408351 | 2.82 | <= |
| | 1454 | | ALTLKL | | | LKL | | | | | WB |
| | | | KYT | | | | | | | | |
| 1011 | DRB1_ | 1036 | KYTEFL | 5 | 1287 | LEIYFS | 0.993 | Sequence | 0.261212 | 3.81 | <= |
| | 1501 | | EIYFST | | | TLS | | | | | WB |
| | | | LSI | | | | | | | | |
| 1012 | DRB1_ | 1037 | YTEFLE | 4 | 1287 | LEIYFS | 0.993 | Sequence | 0.515095 | 1.6 | <= |
| | 1501 | | IYFSTL | | | TLS | | | | | WB |
| | | | SIN | | | | | | | | |
| 1012 | DRB1_ | 1037 | YTEFLE | 4 | 1287 | LEIYFS | 0.96 | Sequence | 0.24199 | 3.25 | <= |
| | 1503 | | IYFSTL | | | TLS | | | | | WB |
| | | | SIN | | | | | | | | |
| 1013 | DRB1_ | 1038 | TEFLEI | 3 | 1287 | LEIYFS | 0.993 | Sequence | 0.566439 | 1.36 | <= |
| | 1501 | | YFSTLS | | | TLS | | | | | WB |
| | | | INT | | | | | | | | |
| 1013 | DRB1_ | 1038 | TEFLEI | 3 | 1287 | LEIYFS | 0.96 | Sequence | 0.279365 | 2.68 | <= |
| | 1503 | | YFSTLS | | | TLS | | | | | WB |
| | | | INT | | | | | | | | |
| 1014 | DRB1_ | 1039 | EFLEIY | 5 | 1288 | YFSTLS | 0.913 | Sequence | 0.395526 | 2.64 | <= |
| | 0701 | | FSTLSI | | | INT | | | | | WB |
| | | | NTK | | | | | | | | |
| 1014 | DRB1_ | 1039 | EFLEIY | 2 | 1287 | LEIYFS | 0.94 | Sequence | 0.253733 | 3.92 | <= |
| | 1501 | | FSTLSI | | | TLS | | | | | WB |
| | | | NTK | | | | | | | | |
| 1015 | DRB1_ | 1040 | FLEIYF | 4 | 1288 | YFSTL SINT | 0.993 | Sequence | 0.487011 | 1.87 | <= |
| | 0701 | | STLSIN | | | | | | | | WB |
| | | | TKT | | | | | | | | |
| 1015 | DRB1_ | 1040 | FLEIYF | 4 | 1288 | YFSTLS | 0.9 | Sequence | 0.409514 | 3.69 | <= |
| | 0901 | | STLSIN | | | INT | | | | | WB |
| | | | TKT | | | | | | | | |
| 1016 | DRB1_ | 1041 | LEIYFS | 3 | 1288 | YFSTL SINT | 0.993 | Sequence | 0.39477 | 2.65 | <= |
| | 0701 | | TLSINT | | | | | | | | WB |
| | | | KTF | | | | | | | | |
| 1017 | DRB1_ | 1042 | EIYFST | 2 | 1288 | YFSTLS | 1 | Sequence | 0.305896 | 3.79 | <= |
| | 0701 | | LSINTK | | | INT | | | | | WB |
| | | | TFK | | | | | | | | |
| 1024 | DRB1_ | 1043 | SINTKT | 6 | 1289 | FKYFKG | 0.953 | Sequence | 0.312625 | 4.1 | <= |
| | 0803 | | FKYFKG | | | YMD | | | | | WB |
| | | | YMD | | | | | | | | |
| 1025 | DRB1_ | 1044 | INTKTF | 5 | 1289 | FKYFKG | 0.993 | Sequence | 0.431765 | 2.16 | <= |
| | 0103 | | KYFKGY | | | YMD | | | | | WB |
| | | | MDQ | | | | | | | | |
| 1025 | DRB1_ | 1044 | INTKTF | 5 | 1289 | FKYFKG | 1 | Sequence | 0.26569 | 4.68 | <= |
| | 0402 | | KYFKGY | | | YMD | | | | | WB |
| | | | MDQ | | | | | | | | |
| 1025 | DRB1_ | 1044 | INTKTF | 5 | 1289 | FKYFKG | 1 | Sequence | 0.580552 | 1.38 | <= |
| | 0405 | | KYFKGY | | | YMD | | | | | WB |
| | | | MDQ | | | | | | | | |
| 1025 | DRB1_ | 1044 | INTKTF | 5 | 1289 | FKYFKG | 0.993 | Sequence | 0.425664 | 2.36 | <= |
| | 0701 | | KYFKGY | | | YMD | | | | | WB |
| | | | MDQ | | | | | | | | |
| 1025 | DRB1_ | 1044 | INTKTF | 5 | 1289 | FKYFKG | 1 | Sequence | 0.652738 | 1.69 | <= |
| | 0801 | | KYFKGY | | | YMD | | | | | WB |
| | | | MDQ | | | | | | | | |
| 1025 | DRB1_ | 1044 | INTKTF | 5 | 1289 | FKYFKG | 0.993 | Sequence | 0.363296 | 4.69 | <= |
| | 0901 | | KYFKGY | | | YMD | | | | | WB |
| | | | MDQ | | | | | | | | |
| 1025 | DRB1_ | 1044 | INTKTF | 5 | 1289 | FKYFKG | 0.987 | Sequence | 0.518766 | 1.59 | <= |
| | 1501 | | KYFKGY | | | YMD | | | | | WB |
| | | | MDQ | | | | | | | | |
| 1025 | DRB1_ | 1044 | INTKTF | 5 | 1289 | FKYFKG | 1 | Sequence | 0.750601 | 0.67 | <= |
| | 1601 | | KYFKGY | | | YMD | | | | | SB |
| | | | MDQ | | | | | | | | |
| 1025 | DRB1_ | 1044 | INTKTF | 5 | 1289 | FKYFKG | 0.987 | Sequence | 0.443227 | 1.24 | <= |
| | 1503 | | KYFKGY | | | YMD | | | | | WB |
| | | | MDQ | | | | | | | | |
| 1025 | DRB1_ | 1044 | INTKTF | 5 | 1289 | FKYFKG | 0.993 | Sequence | 0.588551 | 0.56 | <= |
| | 0803 | | KYFKGY | | | YMD | | | | | SB |
| | | | MDQ | | | | | | | | |
| 1026 | DRB1_ | 1045 | NTKTFK | 4 | 1289 | FKYFKG | 1 | Sequence | 0.523015 | 1.44 | <= |
| | 0103 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.319588 | 3.59 | <= |
| | 0402 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.395572 | 4.6 | <= |
| | 0403 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.626184 | 1.15 | <= |
| | 0405 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.47166 | 1.99 | <= |
| | 0701 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.68686 | 1.41 | <= |
| | 0801 | | YFKGYM | | | YMD | | | | | WB |
| | | | DQI | | | | | | | | |
| 1026 | DRB1_ | 1045 | NTKTFK | 4 | 1289 | FKYFKG | 0.953 | Sequence | 0.400571 | 3.86 | <= |
| | 0901 | | YFKGYM | | | YMD | | | | | WB |
| | | | DQI | | | | | | | | |
| 1026 | DRB1_ | 1045 | NTKTFK | 4 | 1289 | FKYFKG | 0.987 | Sequence | 0.582902 | 1.28 | <= |
| | 1501 | | YFKGYM | | | YMD | | | | | WB |
| | | | DQI | | | | | | | | |
| 1026 | DRB1_ | 1045 | NTKTFK | 4 | 1289 | FKYFKG | 1 | Sequence | 0.813309 | 0.39 | <= |
| | 1601 | | YFKGYM | | | YMD | | | | | SB |
| | | | DQI | | | | | | | | |
| 1026 | DRB1_ | 1045 | NTKTFK | 4 | 1289 | FKYFKG | 0.987 | Sequence | 0.514251 | 0.89 | <= |
| | 1503 | | YFKGYM | | | YMD | | | | | SB |
| | | | DQI | | | | | | | | |
| 1026 | DRB1_ | 1045 | NTKTFK | 4 | 1289 | FKYFKG | 0.987 | Sequence | 0.625482 | 0.41 | <= |
| | 0803 | | YFKGYM | | | YMD | | | | | SB |
| | | | DQI | | | | | | | | |
| 1027 | DRB1_ | 1046 | TKTFKY | 3 | 1289 | FKYFKG | 0.993 | Sequence | 0.307628 | 4.4 | <= |
| | 0101 | | FKGYMD | | | YMD | | | | | WB |
| | | | QIG | | | | | | | | |
| 1027 | DRB1_ | 1046 | TKTFKY | 3 | 1289 | FKYFKG | 1 | Sequence | 0.622884 | 0.9 | <= |
| | 0103 | | FKGYMD | | | YMD | | | | | SB |
| | | | QIG | | | | | | | | |
| 1027 | DRB1_ | 1046 | TKTFKY | 3 | 1289 | FKYFKG | 0.92 | Sequence | 0.412878 | 2.34 | <= |
| | 0402 | | FKGYMD | | | YMD | | | | | WB |
| | | | QIG | | | | | | | | |
| 1027 | DRB1_ | 1046 | TKTFKY | 3 | 1289 | FKYFKG | 0.973 | Sequence | 0.45259 | 3.59 | <= |
| | 0403 | | FKGYMD | | | YMD | | | | | WB |
| | | | QIG | | | | | | | | |
| 1027 | DRB1_ | 1046 | TKTFKY | 3 | 1289 | FKYFKG | 0.993 | Sequence | 0.687129 | 0.91 | <= |
| | 0405 | | FKGYMD | | | YMD | | | | | SB |
| | | | QIG | | | | | | | | |
| 1027 | DRB1_ | 1046 | TKTFKY | 3 | 1289 | FKYFKG | 1 | Sequence | 0.538784 | 1.52 | <= |
| | 0701 | | FKGYMD | | | YMD | | | | | WB |
| | | | QIG | | | | | | | | |
| 1027 | DRB1_ | 1046 | TKTFKY | 3 | 1289 | FKYFKG | 0.993 | Sequence | 0.748591 | 0.92 | <= |
| | 0801 | | FKGYMD | | | YMD | | | | | SB |
| | | | QIG | | | | | | | | |
| 1027 | DRB1_ | 1046 | TKTFKY | 3 | 1289 | FKYFKG | 0.9 | Sequence | 0.470085 | 2.65 | <= |
| | 0901 | | FKGYMD | | | YMD | | | | | WB |
| | | | QIG | | | | | | | | |
| 1027 | DRB1_ | 1046 | TKTFKY | 3 | 1289 | FKYFKG | 0.98 | Sequence | 0.444842 | 3.83 | <= |
| | 1001 | | FKGYMD | | | YMD | | | | | WB |
| | | | QIG | | | | | | | | |
| 1027 | DRB1_ | 1046 | TKTFKY | 3 | 1289 | FKYFKG | 0.98 | Sequence | 0.662306 | 0.95 | <= |
| | 1501 | | FKGYMD | | | YMD | | | | | SB |
| | | | QIG | | | | | | | | |
| 1027 | DRB1_ | 1046 | TKTFKY | 3 | 1289 | FKYFKG | 1 | Sequence | 0.876494 | 0.2 | <= |
| | 1601 | | FKGYMD | | | YMD | | | | | SB |
| | | | QIG | | | | | | | | |
| 1027 | DRB1_ | 1046 | TKTFKY | 3 | 1289 | FKYFKG | 0.98 | Sequence | 0.59433 | 0.6 | <= |
| | 1503 | | FKGYMD | | | YMD | | | | | SB |
| | | | QIG | | | | | | | | |
| 1027 | DRB1_ | 1046 | TKTFKY | 3 | 1289 | FKYFKG | 1 | Sequence | 0.326696 | 4.58 | <= |
| | 1303 | | FKGYMD | | | YMD | | | | | WB |
| | | | QIG | | | | | | | | |
| 1027 | DRB1_ | 1046 | TKTFKY | 3 | 1289 | FKYFKG | 0.993 | Sequence | 0.667496 | 0.28 | <= |
| | 0803 | | FKGYMD | | | YMD | | | | | SB |
| | | | QIG | | | | | | | | |
| 1027 | DRB1_ | 1046 | TKTFKY | 3 | 1289 | FKYFKG | 0.98 | Sequence | 0.379403 | 3.98 | <= |
| | 0408 | | FKGYMD | | | YMD | | | | | WB |
| | | | QIG | | | | | | | | |
| 1028 | DRB1_ | 1047 | KTFKYF | 2 | 1289 | FKYFKG | 0.987 | Sequence | 0.386599 | 2.6 | <= |
| | 0103 | | KGYMDQ | | | YMD | | | | | WB |
| | | | IGL | | | | | | | | |
| 1028 | DRB1_ | 1047 | KTFKYF | 5 | 1290 | FKGYMD | 0.6 | Sequence | 0.370103 | 2.85 | <= |
| | 0402 | | KGYMDQ | | | QIG | | | | | WB |
| | | | IGL | | | | | | | | |
| 1028 | DRB1_ | 1047 | KTFKYF | 2 | 1289 | FKYFKG | 0.913 | Sequence | 0.452702 | 2.21 | <= |
| | 0405 | | KGYMDQ | | | YMD | | | | | WB |
| | | | IGL | | | | | | | | |
| 1028 | DRB1_ | 1047 | KTFKYF | 2 | 1289 | FKYFKG | 0.98 | Sequence | 0.314405 | 3.66 | <= |
| | 0701 | | KGYMDQ | | | YMD | | | | | WB |
| | | | IGL | | | | | | | | |
| 1028 | DRB1_ | 1047 | KTFKYF | 2 | 1289 | FKYFKG | 0.987 | Sequence | 0.547145 | 2.87 | <= |
| | 0801 | | KGYMDQ | | | YMD | | | | | WB |
| | | | IGL | | | | | | | | |
| 1028 | DRB1_ | 1047 | KTFKYF | 2 | 1289 | FKYFKG | 0.827 | Sequence | 0.457047 | 1.94 | <= |
| | 1501 | | KGYMDQ | | | YMD | | | | | WB |
| | | | IGL | | | | | | | | |
| 1028 | DRB1_ | 1047 | KTFKYF | 2 | 1289 | FKYFKG | 0.987 | Sequence | 0.686349 | 1.01 | <= |
| | 1601 | | KGYMDQ | | | YMD | | | | | WB |
| | | | IGL | | | | | | | | |
| 1028 | DRB1_ | 1047 | KTFKYF | 2 | 1289 | FKYFKG | 0.84 | Sequence | 0.413847 | 1.42 | <= |
| | 1503 | | KGYMDQ | | | YMD | | | | | WB |
| | | | IGL | | | | | | | | |
| 1028 | DRB1_ | 1047 | KTFKYF | 2 | 1289 | FKYFKG | 0.94 | Sequence | 0.510873 | 1.02 | <= |
| | 0803 | | KGYMDQ | | | YMD | | | | | WB |
| | | | IGL | | | | | | | | |
| 1029 | DRB1_ | 1048 | TFKYFK | 4 | 1290 | FKGYMD | 0.98 | Sequence | 0.47675 | 1.74 | <= |
| | 0402 | | GYMDQI | | | QIG | | | | | WB |
| | | | GLE | | | | | | | | |
| 1029 | DRB1_ | 1048 | TFKYFK | 4 | 1290 | FKGYMD | 0.807 | Sequence | 0.286721 | 3.46 | <= |
| | 1501 | | GYMDQI | | | QIG | | | | | WB |
| | | | GLE | | | | | | | | |
| 1029 | DRB_ | 1048 | TFKYFK | 4 | 1290 | FKGYMD | 0.693 | Sequence | 0.258712 | 2.97 | <= |
| | 1503 | | GYMDQI | | | QIG | | | | | WB |
| | | | GLE | | | | | | | | |
| 1030 | DRB1_ | 1049 | FKYFKG | 3 | 1290 | FKGYMD | 1 | Sequence | 0.598744 | 0.97 | <= |
| | 0402 | | YMDQIG | | | QIG | | | | | SB |
| | | | LEP | | | | | | | | |
| 1030 | DRB1_ | 1049 | FKYFKG | 3 | 1290 | FKGYMD | 1 | Sequence | 0.364396 | 2.65 | <= |
| | 1501 | | YMDQIG | | | QIG | | | | | WB |
| | | | LEP | | | | | | | | |
| 1030 | DRB1_ | 1049 | FKYFKG | 3 | 1290 | FKGYMD | 1 | Sequence | 0.310449 | 2.3 | <= |
| | 1503 | | YMDQIG | | | QIG | | | | | WB |
| | | | LEP | | | | | | | | |
| 1031 | DRB1_ | 1050 | KYFKGY | 2 | 1290 | FKGYMD | 1 | Sequence | 0.470467 | 1.78 | <= |
| | 0402 | | MDQIGL | | | QIG | | | | | WB |
| | | | EPV | | | | | | | | |
| 1031 | DRB1_ | 1050 | KYFKGY | 2 | 1290 | FKGYMD | 1 | Sequence | 0.240561 | 4.15 | <= |
| | 1501 | | MDQIGL | | | QIG | | | | | WB |
| | | | EPV | | | | | | | | |
| 1031 | DRB1_ | 1050 | KYFKGY | 2 | 1290 | FKGYMD | 1 | Sequence | 0.211627 | 3.83 | <= |
| | 1503 | | MDQIGL | | | QIG | | | | | WB |
| | | | EPV | | | | | | | | |
| 1037 | DRB1_ | 1051 | MDQIGL | 5 | 1291 | LEPVNV | 0.913 | Sequence | 0.302136 | 3.94 | <= |
| | 0405 | | EPVNVF | | | FAD | | | | | WB |
| | | | ADL | | | | | | | | |
| 1038 | DRB1_ | 1052 | DQIGLE | 4 | 1291 | LEPVNV | 0.973 | Sequence | 0.330605 | 3.49 | <= |
| | 0405 | | PVNVFA | | | FAD | | | | | WB |
| | | | DLL | | | | | | | | |
| 1039 | DRB1_ | 1053 | QIGLEP | 3 | 1291 | LEPVNV | 0.96 | Sequence | 0.466858 | 3.39 | <= |
| | 0403 | | VNVFAD | | | FAD | | | | | WB |
| | | | LLK | | | | | | | | |
| 1039 | DRB1_ | 1053 | QIGLEP | 3 | 1291 | LEPVNV | 1 | Sequence | 0.490797 | 1.92 | <= |
| | 0405 | | VNVFAD | | | FAD | | | | | WB |
| | | | LLK | | | | | | | | |
| 1039 | DRB1_ | 1053 | QIGLEP | 3 | 1291 | LEPVNV | 0.973 | Sequence | 0.285158 | 4.96 | <= |
| | 0803 | | VNVFAD | | | FAD | | | | | WB |
| | | | LLK | | | | | | | | |
| 1042 | DRB1_ | 1054 | LEPVNV | 3 | 1292 | VNVFAD | 1 | Sequence | 0.26254 | 3.8 | <= |
| | 1501 | | FADLLK | | | LLK | | | | | WB |
| | | | SFN | | | | | | | | |
| 1042 | DRB1_ | 1054 | LEPVNV | 3 | 1292 | VNVFAD | 0.993 | Sequence | 0.213518 | 3.8 | <= |
| | 1503 | | FADLLK | | | LLK | | | | | WB |
| | | | SFN | | | | | | | | |
| 1050 | DRB1_ | 1055 | DLLKSF | 5 | 1293 | FNFFNS | 0.96 | Sequence | 0.333646 | 2.93 | <= |
| | 1501 | | NFFNSQ | | | QDR | | | | | WB |
| | | | DRK | | | | | | | | |
| 1050 | DRB1_ | 1055 | DLLKSF | 5 | 1293 | FNFFNS | 1 | Sequence | 0.731644 | 0.76 | <= |
| | 1601 | | NFFNSQ | | | QDR | | | | | SB |
| | | | DRK | | | | | | | | |
| 1050 | DRB1_ | 1055 | DLLKSF | 5 | 1293 | FNFFNS | 0.893 | Sequence | 0.300857 | 2.41 | <= |
| | 1503 | | NFFNSQ | | | QDR | | | | | WB |
| | | | DRK | | | | | | | | |
| 1051 | DRB1_ | 1056 | LLKSFN | 4 | 1293 | FNFFNS | 0.987 | Sequence | 0.304864 | 3.71 | <= |
| | 0103 | | FFNSQD | | | QDR | | | | | WB |
| | | | RKD | | | | | | | | |
| 1051 | DRB1_ | 1056 | LLKSFN | 4 | 1293 | FNFFNS | 0.967 | Sequence | 0.292467 | 4.08 | <= |
| | 0402 | | FFNSQD | | | QDR | | | | | WB |
| | | | RKD | | | | | | | | |
| 1051 | DRB1_ | 1056 | LLKSFN | 4 | 1293 | FNFFNS | 1 | Sequence | 0.553957 | 2.88 | <= |
| | 1301 | | FFNSQD | | | QDR | | | | | WB |
| | | | RKD | | | | | | | | |
| 1051 | DRB1_ | 1056 | LLKSFN | 4 | 1293 | FNFFNS | 0.993 | Sequence | 0.511915 | 1.62 | <= |
| | 1501 | | FFNSQD | | | QDR | | | | | WB |
| | | | RKD | | | | | | | | |
| 1051 | DRB1_ | 1056 | LLKSFN | 4 | 1293 | FNFFNS | 1 | Sequence | 0.859019 | 0.25 | <= |
| | 1601 | | FFNSQD | | | QDR | | | | | SB |
| | | | RKD | | | | | | | | |
| 1051 | DRB1_ | 1056 | LLKSFN | 4 | 1293 | FNFFNS | 0.987 | Sequence | 0.440076 | 1.26 | <= |
| | 1503 | | FFNSQD | | | QDR | | | | | WB |
| | | | RKD | | | | | | | | |
| 1052 | DRB1_ | 1057 | LKSFNF | 3 | 1293 | FNFFNS | 0.987 | Sequence | 0.357991 | 2.93 | <= |
| | 0103 | | FNSQDR | | | QDR | | | | | WB |
| | | | KDS | | | | | | | | |
| 1052 | DRB1_ | 1057 | LKSFNF | 3 | 1293 | FNFFNS | 0.88 | Sequence | 0.343196 | 3.22 | <= |
| | 0402 | | FNSQDR | | | QDR | | | | | WB |
| | | | KDS | | | | | | | | |
| 1052 | DRB1_ | 1057 | LKSFNF | 5 | 1294 | FFNSQD | 0.753 | Sequence | 0.420487 | 4.14 | <= |
| | 0403 | | FNSQDR | | | RKD | | | | | WB |
| | | | KDS | | | | | | | | |
| 1052 | DRB1_ | 1057 | LKSFNF | 5 | 1294 | FFNSQD | 0.86 | Sequence | 0.530859 | 1.66 | <= |
| | 0405 | | FNSQDR | | | RKD | | | | | WB |
| | | | KDS | | | | | | | | |
| 1052 | DRB1_ | 1057 | LKSFNF | 3 | 1293 | FNFFNS | 1 | Sequence | 0.607179 | 2.26 | <= |
| | 1301 | | FNSQDR | | | QDR | | | | | WB |
| | | | KDS | | | | | | | | |
| 1052 | DRB1_ | 1057 | LKSFNF | 3 | 1293 | FNFFNS | 1 | Sequence | 0.557415 | 1.4 | <= |
| | 1501 | | FNSQDR | | | QDR | | | | | WB |
| | | | KDS | | | | | | | | |
| 1052 | DRB1_ | 1057 | LKSFNF | 3 | 1293 | FNFFNS | 1 | Sequence | 0.883685 | 0.18 | <= |
| | 1601 | | FNSQDR | | | QDR | | | | | SB |
| | | | KDS | | | | | | | | |
| 1052 | DRB1_ | 1057 | LKSFNF | 3 | 1293 | FNFFNS | 1 | Sequence | 0.483881 | 1.03 | <= |
| | 1503 | | FNSQDR | | | QDR | | | | | WB |
| | | | KDS | | | | | | | | |
| 1052 | DRB1_ | 1057 | LKSFNF | 3 | 1293 | FNFFNS | 0.94 | Sequence | 0.322298 | 4.16 | <= |
| | 1302 | | FNSQDR | | | QDR | | | | | WB |
| | | | KDS | | | | | | | | |
| 1052 | DRB1_ | 1057 | LKSFNF | 3 | 1293 | FNFFNS | 0.587 | Sequence | 0.323346 | 3.8 | <= |
| | 0803 | | FNSQDR | | | QDR | | | | | WB |
| | | | KDS | | | | | | | | |
| 1053 | DRB1_ | 1058 | KSFNFF | 2 | 1293 | FNFFNS | 0.933 | Sequence | 0.266479 | 4.38 | <= |
| | 0103 | | NSQDRK | | | QDR | | | | | WB |
| | | | DSL | | | | | | | | |
| 1053 | DRB1_ | 1058 | KSFNFF | 2 | 1293 | FNFFNS | 0.82 | Sequence | 0.261344 | 4.78 | <= |
| | 0402 | | NSQDRK | | | QDR | | | | | WB |
| | | | DSL | | | | | | | | |
| 1053 | DRB1_ | 1058 | KSFNFF | 4 | 1294 | FFNSQD | 0.893 | Sequence | 0.433893 | 3.9 | <= |
| | 0403 | | NSQDRK | | | RKD | | | | | WB |
| | | | DSL | | | | | | | | |
| 1053 | DRB1_ | 1058 | KSFNFF | 4 | 1294 | FFNSQD | 0.933 | Sequence | 0.556508 | 1.49 | <= |
| | 0405 | | NSQDRK | | | RKD | | | | | WB |
| | | | DSL | | | | | | | | |
| 1053 | DRB1_ | 1058 | KSFNFF | 2 | 1293 | FNFFNS | 0.973 | Sequence | 0.517574 | 3.39 | <= |
| | 1301 | | NSQDRK | | | QDR | | | | | WB |
| | | | DSL | | | | | | | | |
| 1053 | DRB1_ | 1058 | KSFNFF | 2 | 1293 | FNFFNS | 1 | Sequence | 0.441563 | 2.04 | <= |
| | 1501 | | NSQDRK | | | QDR | | | | | WB |
| | | | DSL | | | | | | | | |
| 1053 | DRB1_ | 1058 | KSFNFF | 2 | 1293 | FNFFNS | 1 | Sequence | 0.827161 | 0.35 | <= |
| | 1601 | | NSQDRK | | | QDR | | | | | SB |
| | | | DSL | | | | | | | | |
| 1053 | DRB1_ | 1058 | KSFNFF | 2 | 1293 | FNFFNS | 1 | Sequence | 0.383209 | 1.63 | <= |
| | 1503 | | NSQDRK | | | QDR | | | | | WB |
| | | | DSL | | | | | | | | |
| 1054 | DRB1_ | 1059 | SFNFFN | 3 | 1294 | FFNSQD | 0.987 | Sequence | 0.411611 | 4.3 | <= |
| | 0403 | | SQDRKD | | | RKD | | | | | WB |
| | | | SLF | | | | | | | | |
| 1054 | DRB1_ | 1059 | SFNFFN | 3 | 1294 | FFNSQD | 1 | Sequence | 0.542083 | 1.58 | <= |
| | 0405 | | SQDRKD | | | RKD | | | | | WB |
| | | | SLF | | | | | | | | |
| 1054 | DRB1_ | 1059 | SFNFFN | 1 | 1293 | FNFFNS | 0.967 | Sequence | 0.382471 | 3.96 | <= |
| | 1601 | | SQDRKD | | | QDR | | | | | WB |
| | | | SLF | | | | | | | | |
| 1055 | DRB1_ | 1060 | FNFFNS | 2 | 1294 | FFNSQD | 1 | Sequence | 0.259476 | 4.72 | <= |
| | 0405 | | QDRKDS | | | RKD | | | | | WB |
| | | | LFK | | | | | | | | |
| 1055 | DRB1_ | 1060 | FNFFNS | 3 | 1295 | FNSQDR | 1 | Sequence | 0.459744 | 3.52 | <= |
| | 1101 | | QDRKDS | | | KDS | | | | | WB |
| | | | LFK | | | | | | | | |
| 1062 | DRB1_ | 1061 | DRKDSL | 5 | 1296 | LFKIKK | 0.947 | Sequence | 0.543317 | 2.92 | <= |
| | 0801 | | FKIKKF | | | FSS | | | | | WB |
| | | | SSG | | | | | | | | |
| 1062 | DRB1_ | 1061 | DRKDSL | 5 | 1296 | LFKIKK | 1 | Sequence | 0.757762 | 1.18 | <= |
| | 1101 | | FKIKKF | | | FSS | | | | | WB |
| | | | SSG | | | | | | | | |
| 1062 | DRB1_ | 1061 | DRKDSL | 5 | 1296 | LFKIKK | 1 | Sequence | 0.872829 | 0.49 | <= |
| | 1104 | | FKIKKF | | | FSS | | | | | SB |
| | | | SSG | | | | | | | | |
| 1062 | DRB1_ | 1061 | DRKDSL | 5 | 1296 | LFKIKK | 1 | Sequence | 0.634706 | 1.96 | <= |
| | 1301 | | FKIKKF | | | FSS | | | | | WB |
| | | | SSG | | | | | | | | |
| 1062 | DRB1_ | 1061 | DRKDSL | 5 | 1296 | LFKIKK | 1 | Sequence | 0.411741 | 2.77 | <= |
| | 1401 | | FKIKKF | | | FSS | | | | | WB |
| | | | SSG | | | | | | | | |
| 1062 | DRB1_ | 1061 | DRKDSL | 5 | 1296 | LFKIKK | 1 | Sequence | 0.411741 | 2.77 | <= |
| | 1454 | | FKIKKF | | | FSS | | | | | WB |
| | | | SSG | | | | | | | | |
| 1063 | DRB1_ | 1062 | RKDSLF | 4 | 1296 | LFKIKK | 0.92 | Sequence | 0.592282 | 2.32 | <= |
| | 0801 | | KIKKFS | | | FSS | | | | | WB |
| | | | SGF | | | | | | | | |
| 1063 | DRB1_ | 1062 | RKDSLF | 4 | 1296 | LFKIKK | 1 | Sequence | 0.820044 | 0.85 | <= |
| | 1101 | | KIKKFS | | | FSS | | | | | SB |
| | | | SGF | | | | | | | | |
| 1063 | DRB1_ | 1062 | RKDSLF | 4 | 1296 | LFKIKK | 1 | Sequence | 0.909381 | 0.33 | <= |
| | 1104 | | KIKKFS | | | FSS | | | | | SB |
| | | | SGF | | | | | | | | |
| 1063 | DRB1_ | 1062 | RKDSLF | 4 | 1296 | LFKIKK | 1 | Sequence | 0.710015 | 1.23 | <= |
| | 1301 | | KIKKFS | | | FSS | | | | | WB |
| | | | SGF | | | | | | | | |
| 1063 | DRB1_ | 1062 | RKDSLF | 4 | 1296 | LFKIKK | 0.993 | Sequence | 0.452138 | 2.24 | <= |
| | 1401 | | KIKKFS | | | FSS | | | | | WB |
| | | | SGF | | | | | | | | |
| 1063 | DRB1_ | 1062 | RKDSLF | 4 | 1296 | LFKIKK | 0.993 | Sequence | 0.452138 | 2.24 | <= |
| | 1454 | | KIKKFS | | | FSS | | | | | WB |
| | | | SGF | | | | | | | | |
| 1063 | DRB1_ | 1062 | RKDSLF | 4 | 1296 | LFKIKK | 0.967 | Sequence | 0.17338 | 4.1 | <= |
| | 1402 | | KIKKFS | | | FSS | | | | | WB |
| | | | SGF | | | | | | | | |
| 1063 | DRB1_ | 1062 | RKDSLF | 4 | 1296 | LFKIKK | 1 | Sequence | 0.328476 | 4.55 | <= |
| | 1303 | | KIKKFS | | | FSS | | | | | WB |
| | | | SGF | | | | | | | | |
| 1063 | DRB1_ | 1062 | RKDSLF | 4 | 1296 | LFKIKK | 0.94 | Sequence | 0.334743 | 3.78 | <= |
| | 1302 | | KIKKFS | | | FSS | | | | | WB |
| | | | SGF | | | | | | | | |
| 1063 | DRB1_ | 1062 | RKDSLF | 4 | 1296 | LFKIKK | 0.76 | Sequence | 0.310295 | 4.17 | <= |
| | 0803 | | KIKKFS | | | FSS | | | | | WB |
| | | | SGF | | | | | | | | |
| 1064 | DRB1_ | 1063 | KDSLFK | 3 | 1296 | LFKIKK | 0.887 | Sequence | 0.688259 | 1.4 | <= |
| | 0801 | | IKKFSS | | | FSS | | | | | WB |
| | | | GFK | | | | | | | | |
| 1064 | DRB1_ | 1063 | KDSLFK | 3 | 1296 | LFKIKK | 1 | Sequence | 0.89138 | 0.48 | <= |
| | 1101 | | IKKFSS | | | FSS | | | | | SB |
| | | | GFK | | | | | | | | |
| 1064 | DRB1_ | 1063 | KDSLFK | 3 | 1296 | LFKIKK | 1 | Sequence | 0.944074 | 0.16 | <= |
| | 1104 | | IKKFSS | | | FSS | | | | | SB |
| | | | GFK | | | | | | | | |
| 1064 | DRB1_ | 1063 | KDSLFK | 3 | 1296 | LFKIKK | 0.987 | Sequence | 0.802557 | 0.6 | <= |
| | 1301 | | IKKFSS | | | FSS | | | | | SB |
| | | | GFK | | | | | | | | |
| 1064 | DRB1_ | 1063 | KDSLFK | 3 | 1296 | LFKIKK | 0.993 | Sequence | 0.513421 | 1.62 | <= |
| | 1401 | | IKKFSS | | | FSS | | | | | WB |
| | | | GFK | | | | | | | | |
| 1064 | DRB1_ | 1063 | KDSLFK | 3 | 1296 | LFKIKK | 0.993 | Sequence | 0.513421 | 1.62 | <= |
| | 1454 | | IKKFSS | | | FSS | | | | | WB |
| | | | GFK | | | | | | | | |
| 1064 | DRB1_ | 1063 | KDSLFK | 3 | 1296 | LFKIKK | 0.967 | Sequence | 0.242823 | 1.93 | <= |
| | 1402 | | IKKFSS | | | FSS | | | | | WB |
| | | | GFK | | | | | | | | |
| 1064 | DRB1_ | 1063 | KDSLFK | 3 | 1296 | LFKIKK | 1 | Sequence | 0.438968 | 3.04 | <= |
| | 1303 | | IKKFSS | | | FSS | | | | | WB |
| | | | GFK | | | | | | | | |
| 1064 | DRB1_ | 1063 | KDSLFK | 3 | 1296 | LFKIKK | 0.94 | Sequence | 0.42917 | 1.82 | <= |
| | 1302 | | IKKFSS | | | FSS | | | | | WB |
| | | | GFK | | | | | | | | |
| 1064 | DRB1_ | 1063 | KDSLFK | 3 | 1296 | LFKIKK | 0.707 | Sequence | 0.398162 | 2.28 | <= |
| | 0803 | | IKKFSS | | | FSS | | | | | WB |
| | | | GFK | | | | | | | | |
| 1065 | DRB1_ | 1064 | DSLFKI | 2 | 1296 | LFKIKK | 0.973 | Sequence | 0.616102 | 2.07 | <= |
| | 1101 | | KKFSSG | | | FSS | | | | | WB |
| | | | FKF | | | | | | | | |
| 1065 | DRB1_ | 1064 | DSLFKI | 2 | 1296 | LFKIKK | 1 | Sequence | 0.809243 | 0.84 | <= |
| | 1104 | | KKFSSG | | | FSS | | | | | SB |
| | | | FKF | | | | | | | | |
| 1065 | DRB1_ | 1064 | DSLFKI | 2 | 1296 | LFKIKK | 0.94 | Sequence | 0.563461 | 2.76 | <= |
| | 1301 | | KKFSSG | | | FSS | | | | | WB |
| | | | FKF | | | | | | | | |
| 1079 | DRB1_ | 1065 | FNFYDW | 5 | 1297 | WKFVGE | 0.967 | Sequence | 0.249456 | 4.78 | <= |
| | 0701 | | KFVGEY | | | YNL | | | | | WB |
| | | | NLE | | | | | | | | |
| 1079 | DRB1_ | 1065 | FNFYDW | 5 | 1297 | WKFVGE | 0.96 | Sequence | 0.380692 | 4.28 | <= |
| | 0901 | | KFVGEY | | | YNL | | | | | WB |
| | | | NLE | | | | | | | | |
| 1080 | DRB1_ | 1066 | NFYDWK | 4 | 1297 | WKFVGE | 0.993 | Sequence | 0.4292 | 2.33 | <= |
| | 0701 | | FVGEYN | | | YNL | | | | | WB |
| | | | LEP | | | | | | | | |
| 1080 | DRB1_ | 1066 | NFYDWK | 4 | 1297 | WKFVGE | 0.98 | Sequence | 0.528597 | 1.9 | <= |
| | 0901 | | FVGEYN | | | YNL | | | | | WB |
| | | | LEP | | | | | | | | |
| 1081 | DRB1_ | 1067 | FYDWKF | 5 | 1298 | FVGEYN | 0.953 | Sequence | 0.350557 | 3.69 | <= |
| | 0401 | | VGEYNL | | | LEP | | | | | WB |
| | | | EPD | | | | | | | | |
| 1081 | DRB1_ | 1067 | FYDWKF | 3 | 1297 | WKFVGE | 1 | Sequence | 0.537951 | 1.53 | <= |
| | 0701 | | VGEYNL | | | YNL | | | | | WB |
| | | | EPD | | | | | | | | |
| 1081 | DRB1_ | 1067 | FYDWKF | 3 | 1297 | WKFVGE | 0.987 | Sequence | 0.616503 | 1.11 | <= |
| | 0901 | | VGEYNL | | | YNL | | | | | WB |
| | | | EPD | | | | | | | | |
| 1081 | DRB1_ | 1067 | FYDWKF | 5 | 1298 | FVGEYN | 0.947 | Sequence | 0.335657 | 4.71 | <= |
| | 0408 | | VGEYNL | | | LEP | | | | | WB |
| | | | EPD | | | | | | | | |
| 1082 | DRB1_ | 1068 | YDWKFV | 4 | 1298 | FVGEYN | 0.98 | Sequence | 0.405424 | 2.97 | <= |
| | 0401 | | GEYNLE | | | LEP | | | | | WB |
| | | | PDL | | | | | | | | |
| 1082 | DRB1_ | 1068 | YDWKFV | 2 | 1297 | WKFVGE | 1 | Sequence | 0.394474 | 2.66 | <= |
| | 0701 | | GEYNLE | | | YNL | | | | | WB |
| | | | PDL | | | | | | | | |
| 1082 | DRB1_ | 1068 | YDWKFV | 2 | 1297 | WKFVGE | 0.967 | Sequence | 0.520411 | 2 | <= |
| | 0901 | | GEYNLE | | | YNL | | | | | WB |
| | | | PDL | | | | | | | | |
| 1082 | DRB1_ | 1068 | YDWKFV | 4 | 1298 | FVGEYN | 0.973 | Sequence | 0.383262 | 3.92 | <= |
| | 0408 | | GEYNLE | | | LEP | | | | | WB |
| | | | PDL | | | | | | | | |
| 1083 | DRB1_ | 1069 | DWKFVG | 3 | 1298 | FVGEYN | 1 | Sequence | 0.323917 | 4.11 | <= |
| | 0401 | | EYNLEP | | | LEP | | | | | WB |
| | | | DLL | | | | | | | | |
| 1085 | DRB1_ | 1070 | KFVGEY | 5 | 1299 | YNLEPD | 0.98 | Sequence | 0.414455 | 2.88 | <= |
| | 0401 | | NLEPDL | | | LLI | | | | | WB |
| | | | LIG | | | | | | | | |
| 1085 | DRB1_ | 1070 | KFVGEY | 5 | 1299 | YNLEPD | 0.993 | Sequence | 0.338469 | 3.39 | <= |
| | 0405 | | NLEPDL | | | LLI | | | | | WB |
| | | | LIG | | | | | | | | |
| 1085 | DRB1_ | 1070 | KFVGEY | 5 | 1299 | YNLEPD | 0.973 | Sequence | 0.37126 | 4.11 | <= |
| | 0408 | | NLEPDL | | | LLI | | | | | WB |
| | | | LIG | | | | | | | | |
| 1086 | DRB1_ | 1071 | FVGEYN | 4 | 1299 | YNLEPD | 1 | Sequence | 0.482794 | 2.67 | <= |
| | 0101 | | LEPDLL | | | LLI | | | | | WB |
| | | | IGS | | | | | | | | |
| 1086 | DRB1_ | 1071 | FVGEYN | 4 | 1299 | YNLEPD | 0.867 | Sequence | 0.122655 | 4.01 | <= |
| | 0305 | | LEPDLL | | | LLI | | | | | WB |
| | | | IGS | | | | | | | | |
| 1086 | DRB1_ | 1071 | FVGEYN | 4 | 1299 | YNLEPD | 1 | Sequence | 0.598876 | 1.37 | <= |
| | 0401 | | LEPDLL | | | LLI | | | | | WB |
| | | | IGS | | | | | | | | |
| 1086 | DRB1_ | 1071 | FVGEYN | 4 | 1299 | YNLEPD | 1 | Sequence | 0.452195 | 2.22 | <= |
| | 0405 | | LEPDLL | | | LLI | | | | | WB |
| | | | IGS | | | | | | | | |
| 1086 | DRB1_ | 1071 | FVGEYN | 4 | 1299 | YNLEPD | 1 | Sequence | 0.428335 | 3.36 | <= |
| | 1601 | | LEPDLL | | | LLI | | | | | WB |
| | | | IGS | | | | | | | | |
| 1086 | DRB1_ | 1071 | FVGEYN | 4 | 1299 | YNLEPD | 1 | Sequence | 0.541007 | 2.15 | <= |
| | 0408 | | LEPDLL | | | LLI | | | | | WB |
| | | | IGS | | | | | | | | |
| 1087 | DRB1_ | 1072 | VGEYNL | 3 | 1299 | YNLEPD | 1 | Sequence | 0.636983 | 1.68 | <= |
| | 0101 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.279128 | 4.15 | <= |
| | 0103 | | EPDLLI | | | LLI | | | | | WB |
| | | | GSD | | | | | | | | |
| 1087 | DRB1_ | 1072 | VGEYNL | 3 | 1299 | YNLEPD | 0.78 | Sequence | 0.177335 | 2.8 | <= |
| | 0305 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.71772 | 0.75 | <= |
| | 0401 | | EPDLLI | | | LLI | | | | | SB |
| | | | GSD | | | | | | | | |
| 1087 | DRB1_ | 1072 | VGEYNL | 3 | 1299 | YNLEPD | 0.973 | Sequence | 0.440088 | 3.8 | <= |
| | 0403 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.5539 | 1.5 | <= |
| | 0405 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.267768 | 4.42 | <= |
| | 0701 | | EPDLLI | | | LLI | | | | | WB |
| | | | GSD | | | | | | | | |
| 1087 | DRB1_ | 1072 | VGEYNL | 3 | 1299 | YNLEPD | 0.987 | Sequence | 0.392848 | 4.02 | <= |
| | 0901 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.450015 | 3.74 | <= |
| | 1001 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.551309 | 1.98 | <= |
| | 1601 | | EPDLLI | | | LLI | | | | | WB |
| | | | GSD | | | | | | | | |
| 1087 | DRB1_ | 1072 | VGEYNL | 3 | 1299 | YNLEPD | 0.873 | Sequence | 0.168603 | 4.31 | <= |
| | 1402 | | EPDLLI | | | LLI | | | | | WB |
| | | | GSD | | | | | | | | |
| 1087 | DRB1_ | 1072 | VGEYNL | 3 | 1299 | YNLEPD | 1 | Sequence | 0.656024 | 1.26 | <= |
| | 0408 | | EPDLLI | | | LLI | | | | | WB |
| | | | GSD | | | | | | | | |
| 1088 | DRB1_ | 1073 | GEYNLE | 2 | 1299 | YNLEPD | 1 | Sequence | 0.335597 | 4.08 | <= |
| | 0101 | | PDLLIG | | | LLI | | | | | WB |
| | | | SDG | | | | | | | | |
| 1088 | DRB1_ | 1073 | GEYNLE | 2 | 1299 | YNLEPD | 0.507 | Sequence | 0.106668 | 4.55 | <= |
| | 0305 | | PDLLIG | | | LLI | | | | | WB |
| | | | SDG | | | | | | | | |
| 1088 | DRB1_ | 1073 | GEYNLE | 2 | 1299 | YNLEPD | 0.98 | Sequence | 0.456449 | 2.47 | <= |
| | 0401 | | PDLLIG | | | LLI | | | | | WB |
| | | | SDG | | | | | | | | |
| 1088 | DRB1_ | 1073 | GEYNLE | 2 | 1299 | YNLEPD | 0.993 | Sequence | 0.336154 | 3.42 | <= |
| | 0405 | | PDLLIG | | | LLI | | | | | WB |
| | | | SDG | | | | | | | | |
| 1088 | DRB1_ | 1073 | GEYNLE | 2 | 1299 | YNLEPD | 1 | Sequence | 0.398073 | 3.71 | <= |
| | 0408 | | PDLLIG | | | LLI | | | | | WB |
| | | | SDG | | | | | | | | |
| 1099 | DRB1_ | 1074 | GSDGIY | 5 | 1300 | YSPIWR | 0.967 | Sequence | 0.341795 | 4.09 | <= |
| | 1401 | | SPIWRN | | | NNF | | | | | WB |
| | | | NFT | | | | | | | | |
| 1099 | DRB1_ | 1074 | GSDGIY | 5 | 1300 | YSPIWR | 0.967 | Sequence | 0.341795 | 4.09 | <= |
| | 1454 | | SPIWRN | | | NNF | | | | | WB |
| | | | NFT | | | | | | | | |
| 1100 | DRB1_ | 1075 | SDGIYS | 4 | 1300 | YSPIWR | 0.993 | Sequence | 0.378789 | 3.33 | <= |
| | 1401 | | PIWRNN | | | NNF | | | | | WB |
| | | | FTI | | | | | | | | |
| 1100 | DRB1_ | 1075 | SDGIYS | 4 | 1300 | YSPIWR | 0.993 | Sequence | 0.378789 | 3.33 | <= |
| | 1454 | | PIWRNN | | | NNF | | | | | WB |
| | | | FTI | | | | | | | | |
| 1101 | DRB1_ | 1076 | DGIYSP | 3 | 1300 | YSPIWR | 0.953 | Sequence | 0.399688 | 2.95 | <= |
| | 1401 | | IWRNNF | | | NNF | | | | | WB |
| | | | TIY | | | | | | | | |
| 1101 | DRB1_ | 1076 | DGIYSP | 3 | 1300 | YSPIWR | 0.953 | Sequence | 0.399688 | 2.95 | <= |
| | 1454 | | IWRNNF | | | NNF | | | | | WB |
| | | | TIY | | | | | | | | |
| 1101 | DRB1_ | 1076 | DGIYSP | 3 | 1300 | YSPIWR | 0.893 | Sequence | 0.301334 | 4.84 | <= |
| | 1302 | | IWRNNF | | | NNF | | | | | WB |
| | | | TIY | | | | | | | | |
| 1103 | DRB1_ | 1077 | IYSPIW | 5 | 1301 | WRNNFT | 0.7 | Sequence | 0.322517 | 4.15 | <= |
| | 1302 | | RNNFTI | | | IYI | | | | | WB |
| | | | YIS | | | | | | | | |
| 1104 | DRB1_ | 1078 | YSPIWR | 4 | 1301 | WRNNFT | 0.787 | Sequence | 0.342122 | 3.56 | <= |
| | 1302 | | NNFTIY | | | IYI | | | | | WB |
| | | | ISW | | | | | | | | |
| 1105 | DRB1_ | 1079 | SPIWRN | 3 | 1301 | WRNNFT | 0.96 | Sequence | 0.168679 | 4.31 | <= |
| | 1402 | | NFTIYI | | | IYI | | | | | WB |
| | | | SWN | | | | | | | | |
| 1105 | DRB1_ | 1079 | SPIWRN | 3 | 1301 | WRNNFT | 0.887 | Sequence | 0.380293 | 2.65 | <= |
| | 1302 | | NFTIYI | | | IYI | | | | | WB |
| | | | SWN | | | | | | | | |
| 1107 | DRB1_ | 1080 | IWRNNF | 5 | 1302 | FTIYIS | 1 | Sequence | 0.315095 | 3.13 | <= |
| | 1501 | | TIYISW | | | WNF | | | | | WB |
| | | | NFF | | | | | | | | |
| 1107 | DRB1_ | 1080 | IWRNNF | 5 | 1302 | FTIYIS | 0.98 | Sequence | 0.193147 | 4.29 | <= |
| | 1503 | | TIYISW | | | WNF | | | | | WB |
| | | | NFF | | | | | | | | |
| 1108 | DRB1_ | 1081 | WRNNFT | 4 | 1302 | FTIYIS | 1 | Sequence | 0.458065 | 1.93 | <= |
| | 1501 | | IYISWN | | | WNF | | | | | WB |
| | | | FFA | | | | | | | | |
| 1108 | DRB1_ | 1081 | WRNNFT | 4 | 1302 | FTIYIS | 0.987 | Sequence | 0.298629 | 2.43 | <= |
| | 1503 | | IYISWN | | | WNF | | | | | WB |
| | | | FFA | | | | | | | | |
| 1109 | DRB1_ | 1082 | RNNFTI | 3 | 1302 | FTIYIS | 0.92 | Sequence | 0.645161 | 1.01 | <= |
| | 1501 | | YISWNF | | | WNF | | | | | WB |
| | | | FAP | | | | | | | | |
| 1109 | DRB1_ | 1082 | RNNFTI | 3 | 1302 | FTIYIS | 0.987 | Sequence | 0.504368 | 2.45 | <= |
| | 1601 | | YISWNF | | | WNF | | | | | WB |
| | | | FAP | | | | | | | | |
| 1109 | DRB1_ | 1082 | RNNFTI | 3 | 1302 | FTIYIS | 0.947 | Sequence | 0.435104 | 1.29 | <= |
| | 1503 | | YISWNF | | | WNF | | | | | WB |
| | | | FAP | | | | | | | | |
| 1110 | DRB1_ | 1083 | NNFTIY | 2 | 1302 | FTIYIS | 0.787 | Sequence | 0.380449 | 2.51 | <= |
| | 1501 | | ISWNFF | | | WNF | | | | | WB |
| | | | API | | | | | | | | |
| 1110 | DRB1_ | 1083 | NNFTIY | 2 | 1302 | FTIYIS | 0.8 | Sequence | 0.228741 | 3.48 | <= |
| | 1503 | | ISWNFF | | | WNF | | | | | WB |
| | | | API | | | | | | | | |
| 1111 | DRB1_ | 1084 | NFTIYI | 3 | 1303 | IYISWN | 0.853 | Sequence | 0.304395 | 3.26 | <= |
| | 1501 | | SWNFFA | | | FFA | | | | | WB |
| | | | PIK | | | | | | | | |
| 1113 | DRB1_ | 1085 | TIYISW | 5 | 1304 | WNFFAP | 0.893 | Sequence | 0.257965 | 4.54 | <= |
| | 0103 | | NFFAPI | | | IKA | | | | | WB |
| | | | KAS | | | | | | | | |
| 1113 | DRB1_ | 1085 | TIYISW | 5 | 1304 | WNFFAP | 0.827 | Sequence | 0.359501 | 4.78 | <= |
| | 0901 | | NFFAPI | | | IKA | | | | | WB |
| | | | KAS | | | | | | | | |
| 1114 | DRB1_ | 1086 | IYISWN | 4 | 1304 | WNFFAP | 0.987 | Sequence | 0.354645 | 3.87 | <= |
| | 0101 | | FFAPIK | | | IKA | | | | | WB |
| | | | ASF | | | | | | | | |
| 1114 | DRB1_ | 1086 | IYISWN | 4 | 1304 | WNFFAP | 0.94 | Sequence | 0.29751 | 3.83 | <= |
| | 0103 | | FFAPIK | | | IKA | | | | | WB |
| | | | ASF | | | | | | | | |
| 1114 | DRB1_ | 1086 | IYISWN | 4 | 1304 | WNFFAP | 0.98 | Sequence | 0.339915 | 4.65 | <= |
| | 1601 | | FFAPIK | | | IKA | | | | | WB |
| | | | ASF | | | | | | | | |
| 1115 | DRB1_ | 1087 | YISWNF | 3 | 1304 | WNFFAP | 0.993 | Sequence | 0.490853 | 2.6 | <= |
| | 0101 | | FAPIKA | | | IKA | | | | | WB |
| | | | SFE | | | | | | | | |
| 1115 | DRB1_ | 1087 | YISWNF | 3 | 1304 | WNFFAP | 0.993 | Sequence | 0.390732 | 4.31 | <= |
| | 0102 | | FAPIKA | | | IKA | | | | | WB |
| | | | SFE | | | | | | | | |
| 1115 | DRB1_ | 1087 | YISWNF | 3 | 1304 | WNFFAP | 0.98 | Sequence | 0.425566 | 2.22 | <= |
| | 0103 | | FAPIKA | | | IKA | | | | | WB |
| | | | SFE | | | | | | | | |
| 1115 | DRB1_ | 1087 | YISWNF | 3 | 1304 | WNFFAP | 0.933 | Sequence | 0.248845 | 4.79 | <= |
| | 0701 | | FAPIKA | | | IKA | | | | | WB |
| | | | SFE | | | | | | | | |
| 1115 | DRB1_ | 1087 | YISWNF | 3 | 1304 | WNFFAP | 0.827 | Sequence | 0.429774 | 3.32 | <= |
| | 0901 | | FAPIKA | | | IKA | | | | | WB |
| | | | SFE | | | | | | | | |
| 1115 | DRB1_ | 1087 | YISWNF | 3 | 1304 | WNFFAP | 0.947 | Sequence | 0.485426 | 3.21 | <= |
| | 1001 | | FAPIKA | | | IKA | | | | | WB |
| | | | SFE | | | | | | | | |
| 1115 | DRB1_ | 1087 | YISWNF | 3 | 1304 | WNFFAP | 0.973 | Sequence | 0.456413 | 3.01 | <= |
| | 1601 | | FAPIKA | | | IKA | | | | | WB |
| | | | SFE | | | | | | | | |
| 1116 | DRB1_ | 1088 | ISWNFF | 2 | 1304 | WNFFAP | 0.953 | Sequence | 0.279358 | 4.8 | <= |
| | 0101 | | APIKAS | | | IKA | | | | | WB |
| | | | FEN | | | | | | | | |
| 1116 | DRB1_ | 1088 | ISWNFF | 2 | 1304 | WNFFAP | 0.88 | Sequence | 0.278881 | 4.15 | <= |
| | 0103 | | APIKAS | | | IKA | | | | | WB |
| | | | FEN | | | | | | | | |
| 1116 | DRB1_ | 1088 | ISWNFF | 5 | 1305 | FAPIKA | 0.987 | Sequence | 0.363493 | 3.08 | <= |
| | 0405 | | APIKAS | | | SFE | | | | | WB |
| | | | FEN | | | | | | | | |
| 1116 | DRB1_ | 1088 | ISWNFF | 2 | 1304 | WNFFAP | 0.56 | Sequence | 0.416339 | 3.56 | <= |
| | 0901 | | APIKAS | | | IKA | | | | | WB |
| | | | FEN | | | | | | | | |
| 1116 | DRB1_ | 1088 | ISWNFF | 2 | 1304 | WNFFAP | 0.547 | Sequence | 0.462442 | 3.54 | <= |
| | 1001 | | APIKAS | | | IKA | | | | | WB |
| | | | FEN | | | | | | | | |
| 1117 | DRB1_ | 1089 | SWNFFA | 4 | 1305 | FAPIKA | 0.993 | Sequence | 0.445805 | 3.7 | <= |
| | 0403 | | PIKASF | | | SFE | | | | | WB |
| | | | ENN | | | | | | | | |
| 1117 | DRB1_ | 1089 | SWNFFA | 4 | 1305 | FAPIKA | 0.993 | Sequence | 0.569154 | 1.43 | <= |
| | 0405 | | PIKASF | | | SFE | | | | | WB |
| | | | ENN | | | | | | | | |
| 1117 | DRB1_ | 1089 | SWNFFA | 4 | 1305 | FAPIKA | 0.987 | Sequence | 0.571118 | 2.57 | <= |
| | 0801 | | PIKASF | | | SFE | | | | | WB |
| | | | ENN | | | | | | | | |
| 1117 | DRB1_ | 1089 | SWNFFA | 4 | 1305 | FAPIKA | 0.673 | Sequence | 0.37889 | 4.32 | <= |
| | 0901 | | PIKASF | | | SFE | | | | | WB |
| | | | ENN | | | | | | | | |
| 1117 | DRB1_ | 1089 | SWNFFA | 4 | 1305 | FAPIKA | 0.913 | Sequence | 0.528963 | 2.65 | <= |
| | 1001 | | PIKASF | | | SFE | | | | | WB |
| | | | ENN | | | | | | | | |
| 1117 | DRB1_ | 1089 | SWNFFA | 4 | 1305 | FAPIKA | 0.867 | Sequence | 0.346322 | 4.27 | <= |
| | 1303 | | PIKASF | | | SFE | | | | | WB |
| | | | ENN | | | | | | | | |
| 1117 | DRB1_ | 1089 | SWNFFA | 4 | 1305 | FAPIKA | 0.9 | Sequence | 0.351858 | 3.11 | <= |
| | 0803 | | PIKASF | | | SFE | | | | | WB |
| | | | ENN | | | | | | | | |
| 1118 | DRB1_ | 1090 | WNFFAP | 3 | 1305 | FAPIKA | 1 | Sequence | 0.571198 | 2.12 | <= |
| | 0403 | | IKASFE | | | SFE | | | | | WB |
| | | | NNK | | | | | | | | |
| 1118 | DRB1_ | 1090 | WNFFAP | 3 | 1305 | FAPIKA | 1 | Sequence | 0.687058 | 0.91 | <= |
| | 0405 | | IKASFE | | | SFE | | | | | SB |
| | | | NNK | | | | | | | | |
| 1118 | DRB1_ | 1090 | WNFFAP | 3 | 1305 | FAPIKA | 0.98 | Sequence | 0.268734 | 4.4 | <= |
| | 0701 | | IKASFE | | | SFE | | | | | WB |
| | | | NNK | | | | | | | | |
| 1118 | DRB1_ | 1090 | WNFFAP | 3 | 1305 | FAPIKA | 1 | Sequence | 0.717907 | 1.16 | <= |
| | 0801 | | IKASFE | | | SFE | | | | | WB |
| | | | NNK | | | | | | | | |
| 1118 | DRB1_ | 1090 | WNFFAP | 3 | 1305 | FAPIKA | 0.933 | Sequence | 0.40313 | 3.81 | <= |
| | 0901 | | IKASFE | | | SFE | | | | | WB |
| | | | NNK | | | | | | | | |
| 1118 | DRB1_ | 1090 | WNFFAP | 3 | 1305 | FAPIKA | 1 | Sequence | 0.681064 | 1.25 | <= |
| | 1001 | | IKASFE | | | SFE | | | | | WB |
| | | | NNK | | | | | | | | |
| 1118 | DRB1_ | 1090 | WNFFAP | 3 | 1305 | FAPIKA | 0.98 | Sequence | 0.47457 | 2.66 | <= |
| | 1303 | | IKASFE | | | SFE | | | | | WB |
| | | | NNK | | | | | | | | |
| 1118 | DRB1_ | 1090 | WNFFAP | 3 | 1305 | FAPIKA | 0.973 | Sequence | 0.451364 | 1.56 | <= |
| | 0803 | | IKASFE | | | SFE | | | | | WB |
| | | | NNK | | | | | | | | |
| 1118 | DRB1_ | 1090 | WNFFAP | 3 | 1305 | FAPIKA | 1 | Sequence | 0.398607 | 3.71 | <= |
| | 0408 | | IKASFE | | | SFE | | | | | WB |
| | | | NNK | | | | | | | | |
| 1119 | DRB1_ | 1091 | NFFAPI | 2 | 1305 | FAPIKA | 0.993 | Sequence | 0.413958 | 4.26 | <= |
| | 0403 | | KASFEN | | | SFE | | | | | WB |
| | | | NKD | | | | | | | | |
| 1119 | DRB1_ | 1091 | NFFAPI | 2 | 1305 | FAPIKA | 0.993 | Sequence | 0.513944 | 1.77 | <= |
| | 0405 | | KASFEN | | | SFE | | | | | WB |
| | | | NKD | | | | | | | | |
| 1119 | DRB1_ | 1091 | NFFAPI | 2 | 1305 | FAPIKA | 0.993 | Sequence | 0.577359 | 2.49 | <= |
| | 0801 | | KASFEN | | | SFE | | | | | WB |
| | | | NKD | | | | | | | | |
| 1119 | DRB1_ | 1091 | NFFAPI | 2 | 1305 | FAPIKA | 1 | Sequence | 0.541656 | 2.49 | <= |
| | 1001 | | KASFEN | | | SFE | | | | | WB |
| | | | NKD | | | | | | | | |
| 1119 | DRB1_ | 1091 | NFFAPI | 2 | 1305 | FAPIKA | 0.947 | Sequence | 0.326841 | 4.58 | <= |
| | 1303 | | KASFEN | | | SFE | | | | | WB |
| | | | NKD | | | | | | | | |
| 1119 | DRB1_ | 1091 | NFFAPI | 2 | 1305 | FAPIKA | 0.953 | Sequence | 0.326617 | 3.72 | <= |
| | 0803 | | KASFEN | | | SFE | | | | | WB |
| | | | NKD | | | | | | | | |
| 1123 | DRB1_ | 1092 | PIKASF | 5 | 1306 | FENNKD | 0.973 | Sequence | 0.1323 | 3.75 | <= |
| | 0305 | | ENNKDT | | | TNY | | | | | WB |
| | | | NYD | | | | | | | | |
| 1123 | DRB1_ | 1092 | PIKASF | 5 | 1306 | FENNKD | 0.933 | Sequence | 0.164224 | 4.51 | <= |
| | 1402 | | ENNKDT | | | TNY | | | | | WB |
| | | | NYD | | | | | | | | |
| 1123 | DRB1_ | 1092 | PIKASF | 5 | 1306 | FENNKD | 0.987 | Sequence | 0.351884 | 3.3 | <= |
| | 1302 | | ENNKDT | | | TNY | | | | | WB |
| | | | NYD | | | | | | | | |
| 1124 | DRB1_ | 1093 | IKASFE | 4 | 1306 | FENNKD | 0.987 | Sequence | 0.155654 | 3.19 | <= |
| | 0305 | | NNKDTN | | | TNY | | | | | WB |
| | | | YDL | | | | | | | | |
| 1124 | DRB1_ | 1093 | IKASFE | 4 | 1306 | FENNKD | 0.973 | Sequence | 0.18617 | 3.59 | <= |
| | 1402 | | NNKDTN | | | TNY | | | | | WB |
| | | | YDL | | | | | | | | |
| 1124 | DRB1_ | 1093 | IKASFE | 4 | 1306 | FENNKD | 1 | Sequence | 0.395654 | 2.36 | <= |
| | 1302 | | NNKDTN | | | TNY | | | | | WB |
| | | | YDL | | | | | | | | |
| 1125 | DRB1_ | 1094 | KASFEN | 3 | 1306 | FENNKD | 0.987 | Sequence | 0.158858 | 3.12 | <= |
| | 0305 | | NKDTNY | | | TNY | | | | | W |
| | | | DLI | | | | | | | | B |
| 1125 | DRB1_ | 1094 | KASFEN | 3 | 1306 | FENNKD | 0.993 | Sequence | 0.186363 | 3.58 | <= |
| | 1402 | | NKDTNY | | | TNY | | | | | WB |
| | | | DLI | | | | | | | | |
| 1125 | DRB1_ | 1094 | KASFEN | 3 | 1306 | FENNKD | 1 | Sequence | 0.406351 | 2.17 | <= |
| | 1302 | | NKDTNY | | | TNY | | | | | WB |
| | | | DLI | | | | | | | | |
| 1130 | DRB1_ | 1095 | NNKDTN | 4 | 1307 | TNYDLI | 1 | Sequence | 0.249541 | 4.53 | <= |
| | 0301 | | YDLIIN | | | INR | | | | | WB |
| | | | RKT | | | | | | | | |
| 1131 | DRB1_ | 1096 | NKDTNY | 5 | 1308 | YDLIIN | 1 | Sequence | 0.540772 | 1.33 | <= |
| | 0103 | | DLIINR | | | RKT | | | | | WB |
| | | | KTK | | | | | | | | |
| 1131 | DRB1_ | 1096 | NKDTNY | 3 | 1307 | TNYDLI | 1 | Sequence | 0.347254 | 3.27 | <= |
| | 0301 | | DLIINR | | | INR | | | | | WB |
| | | | KTK | | | | | | | | |
| 1131 | DRB1_ | 1096 | NKDTNY | 5 | 1308 | YDLIIN | 1 | Sequence | 0.631037 | 1.37 | <= |
| | 1601 | | DLIINR | | | RKT | | | | | WB |
| | | | KTK | | | | | | | | |
| 1131 | DRB1_ | 1096 | NKDTNY | 5 | 1308 | YDLIIN | 1 | Sequence | 0.378915 | 3.98 | <= |
| | 0408 | | DLIINR | | | RKT | | | | | WB |
| | | | KTK | | | | | | | | |
| 1132 | DRB1_ | 1097 | KDTNYD | 4 | 1308 | YDLIIN | 1 | Sequence | 0.516276 | 2.41 | <= |
| | 0101 | | LIINRK | | | RKT | | | | | WB |
| | | | TKK | | | | | | | | |
| 1132 | DRB1_ | 1097 | KDTNYD | 4 | 1308 | YDLIIN | 1 | Sequence | 0.793214 | 0.33 | <= |
| | 0103 | | LIINRK | | | RKT | | | | | SB |
| | | | TKK | | | | | | | | |
| 1132 | DRB1_ | 1097 | KDTNYD | 4 | 1308 | YDLIIN | 1 | Sequence | 0.496902 | 2.13 | <= |
| | 0401 | | LIINRK | | | RKT | | | | | WB |
| | | | TKK | | | | | | | | |
| 1132 | DRB1_ | 1097 | KDTNYD | 4 | 1308 | YDLIIN | 0.993 | Sequence | 0.364462 | 2.92 | <= |
| | 0402 | | LIINRK | | | RKT | | | | | WB |
| | | | TKK | | | | | | | | |
| 1132 | DRB1_ | 1097 | KDTNYD | 4 | 1308 | YDLIIN | 1 | Sequence | 0.268003 | 4.54 | <= |
| | 0405 | | LIINRK | | | RKT | | | | | WB |
| | | | TKK | | | | | | | | |
| 1132 | DRB1_ | 1097 | KDTNYD | 4 | 1308 | YDLIIN | 0.98 | Sequence | 0.492215 | 3.69 | <= |
| | 0801 | | LIINRK | | | RKT | | | | | WB |
| | | | TKK | | | | | | | | |
| 1132 | DRB1_ | 1097 | KDTNYD | 4 | 1308 | YDLIIN | 1 | Sequence | 0.525019 | 2.69 | <= |
| | 1001 | | LIINRK | | | RKT | | | | | WB |
| | | | TKK | | | | | | | | |
| 1132 | DRB1_ | 1097 | KDTNYD | 4 | 1308 | YDLIIN | 0.987 | Sequence | 0.578217 | 2.37 | <= |
| | 1101 | | LIINRK | | | RKT | | | | | WB |
| | | | TKK | | | | | | | | |
| 1132 | DRB1_ | 1097 | KDTNYD | 4 | 1308 | YDLIIN | 1 | Sequence | 0.830536 | 0.34 | <= |
| | 1601 | | LIINRK | | | RKT | | | | | SB |
| | | | TKK | | | | | | | | |
| 1132 | DRB1_ | 1097 | KDTNYD | 4 | 1308 | YDLIIN | 0.947 | Sequence | 0.29509 | 4.63 | <= |
| | 0803 | | LIINRK | | | RKT | | | | | WB |
| | | | TKK | | | | | | | | |
| 1132 | DRB1_ | 1097 | KDTNYD | 4 | 1308 | YDLIIN | 1 | Sequence | 0.627323 | 1.44 | <= |
| | 0408 | | LIINRK | | | RKT | | | | | WB |
| | | | TKK | | | | | | | | |

Table 4 below shows predicted human MHCII binding of peptides derived from BB0838 determined using NetMHCIIpan (V 4.0). The prediction outcome for each molecule includes the following columns in the table: Pos Residue number (starting from 923); MI-C molecule name; Peptide Amino acid sequence; Of Starting position offset of the optimal binding core (starting from 0); Core Binding core register; Core_Rel Reliability of the binding core, expressed as the fraction of networks in the ensemble selecting the optimal core; Score_EL Eluted ligand prediction score; % Rank_EL Percentile rank of eluted ligand prediction score; BindLevel (SB: strong binder, WB: weak binder). The peptide will be identified as a strong binder if the % Rank is below the 1% threshold for the strong binders. The peptide will be identified as a weak binder if the 0% Rank is above the 1% threshold of the strong binders but below the 50% threshold for the weak binders.

TABLE 4
Predicted human CD4+ T cell epitopes within B. burgdorferi LptD 922-972

| | | SEQ | | | SEQ | | | | | | |
|---|---|---|---|---|---|---|---|---|---|---|---|
| | | ID | | | ID | | Core_ | | Score_ | %Rank_ | Bind |
| Pos | MHC | NO: | Peptide | Of | NO: | Core | Rel | Identity | EL | EL | Level |
| 923 | DRB1_ | 1000 | NVFDFQFLF | 4 | 1274 | FQFLFAM | 0.993 | Sequence | 0.305403 | 3.89 | ⇐WB |
| | 0405 | | AMKDEF | | | KD | | | | | |
| 923 | DRB1_ | 1000 | NVFDFQFLF | 4 | 1274 | FQFLFAM | 1 | Sequence | 0.510395 | 2.87 | ⇐WB |
| | 1001 | | AMKDEF | | | KD | | | | | |
| 924 | DRB1_ | 1001 | VFDFQFLFA | 3 | 1274 | FQFLFAM | 1 | Sequence | 0.27607 | 4.85 | ⇐WB |
| | 0101 | | MKDEFE | | | KD | | | | | |
| 924 | DRB1_ | 1001 | VFDFQFLFA | 3 | 1274 | FQFLFAM | 0.9 | Sequence | 0.428955 | 2.41 | ⇐WB |
| | 0405 | | MKDEFE | | | KD | | | | | |
| 924 | DRB1_ | 1001 | VFDFQFLFA | 3 | 1274 | FQFLFAM | 1 | Sequence | 0.621245 | 1.71 | ⇐WB |
| | 1001 | | MKDEFE | | | KD | | | | | |
| 925 | DRB1_ | 1002 | FDFQFLFAM | 5 | 1275 | LFAMKD | 0.86 | Sequence | 0.486759 | 3.13 | ⇐WB |
| | 0403 | | KDEFEY | | | EFE | | | | | |
| 925 | DRB1_ | 1002 | FDFQFLFAM | 5 | 1275 | LFAMKD | 0.747 | Sequence | 0.486413 | 1.95 | ⇐WB |
| | 0405 | | KDEFEY | | | EFE | | | | | |
| 925 | DRB1_ | 1002 | FDFQFLFAM | 2 | 1274 | FQFLFAM | 0.987 | Sequence | 0.448017 | 3.78 | ⇐WB |
| | 1001 | | KDEFEY | | | KD | | | | | |
| 926 | DRB1_ | 1003 | DFQFLFAMK | 4 | 1275 | LFAMKD | 0.987 | Sequence | 0.680627 | 1.17 | ⇐WB |
| | 0403 | | DEFEYN | | | EFE | | | | | |
| 926 | DRB1_ | 1003 | DFQFLFAMK | 4 | 1275 | LFAMKD | 0.993 | Sequence | 0.366935 | 4.32 | ⇐WB |
| | 0404 | | DEFEYN | | | EFE | | | | | |
| 926 | DRB1_ | 1003 | DFQFLFAMK | 4 | 1275 | LFAMKD | 0.98 | Sequence | 0.64421 | 1.08 | ⇐WB |
| | 0405 | | DEFEYN | | | EFE | | | | | |
| 926 | DRB1_ | 1003 | DFQFLFAMK | 3 | 1276 | FLFAMK | 0.893 | Sequence | 0.333476 | 4.29 | ⇐WB |
| | 1401 | | DEFEYN | | | DEF | | | | | |
| 926 | DRB1_ | 1003 | DFQFLFAMK | 3 | 1276 | FLFAMK | 0.893 | Sequence | 0.333476 | 4.29 | ⇐WB |
| | 1454 | | DEFEYN | | | DEF | | | | | |
| 927 | DRB1_ | 1004 | FQFLFAMKD | 3 | 1275 | LFAMKD | 1 | Sequence | 0.693216 | 1.09 | ⇐WB |
| | 0403 | | EFEYNY | | | EFE | | | | | |
| 927 | DRB1_ | 1004 | FQFLFAMKD | 3 | 1275 | LFAMKD | 0.993 | Sequence | 0.417478 | 3.62 | ⇐WB |
| | 0404 | | EFEYNY | | | EFE | | | | | |
| 927 | DRB1_ | 1004 | FQFLFAMKD | 3 | 1275 | LFAMKD | 1 | Sequence | 0.651081 | 1.05 | ⇐WB |
| | 0405 | | EFEYNY | | | EFE | | | | | |
| 928 | DRB1_ | 1005 | QFLFAMKDE | 2 | 1275 | LFAMKD | 1 | Sequence | 0.537304 | 2.47 | ⇐WB |
| | 0403 | | FEYNYD | | | EFE | | | | | |
| 928 | DRB1_ | 1005 | QFLFAMKDE | 2 | 1275 | LFAMKD | 1 | Sequence | 0.467705 | 2.09 | ⇐WB |
| | 0405 | | FEYNYD | | | EFE | | | | | |
| 933 | DRB1_ | 1006 | MKDEFEYN | 4 | 1277 | FEYNYDP | 0.827 | Sequence | 0.172456 | 2.88 | ⇐WB |
| | 0305 | | YDPLKGD | | | LK | | | | | |
| 933 | DRB1_ | 1006 | MKDEFEYN | 4 | 1277 | FEYNYDP | 0.973 | Sequence | 0.285239 | 4.82 | ⇐WB |
| | 0401 | | YDPLKGD | | | LK | | | | | |
| 933 | DRB1_ | 1006 | MKDEFEYN | 4 | 1277 | FEYNYDP | 0.927 | Sequence | 0.200938 | 3.05 | ⇐WB |
| | 1402 | | YDPLKGD | | | LK | | | | | |
| 934 | DRB1_ | 1007 | KDEFEYNYD | 3 | 1277 | FEYNYDP | 0.633 | Sequence | 0.199042 | 2.48 | ⇐WB |
| | 0305 | | PLKGDF | | | LK | | | | | |
| 934 | DRB1_ | 1007 | KDEFEYNYD | 3 | 1277 | FEYNYDP | 0.84 | Sequence | 0.289904 | 4.72 | ⇐WB |
| | 0401 | | PLKGDF | | | LK | | | | | |
| 934 | DRB1_ | 1007 | KDEFEYNYD | 3 | 1277 | FEYNYDP | 0.907 | Sequence | 0.300157 | 4.88 | ⇐WB |
| | 1302 | | PLKGDF | | | LK | | | | | |
| 934 | DRB1_ | 1007 | KDEFEYNYD | 5 | 1278 | YNYDPL | 0.76 | Sequence | 0.420759 | 3.26 | ⇐WB |
| | 1303 | | PLKGDF | | | KGD | | | | | |
| 934 | DRB1_ | 1007 | KDEFEYNYD | 3 | 1277 | FEYNYDP | 0.84 | Sequence | 0.217644 | 2.57 | ⇐WB |
| | 1402 | | PLKGDF | | | LK | | | | | |
| 935 | DRB1_ | 1008 | DEFEYNYDP | 4 | 1278 | YNYDPL | 0.667 | Sequence | 0.160477 | 3.09 | ⇐WB |
| | 0305 | | LKGDFS | | | KGD | | | | | |
| 935 | DRB1_ | 1008 | DEFEYNYDP | 4 | 1278 | YNYDPL | 0.947 | Sequence | 0.524326 | 2.16 | ⇐WB |
| | 1303 | | LKGDFS | | | KGD | | | | | |
| 936 | DRB1_ | 1009 | EFEYNYDPL | 5 | 1279 | YDPLKG | 0.993 | Sequence | 0.412952 | 3.29 | ⇐WB |
| | 0101 | | KGDFSK | | | DFS | | | | | |
| 936 | DRB1_ | 1009 | EFEYNYDPL | 3 | 1278 | YNYDPL | 0.953 | Sequence | 0.177471 | 2.8 | ⇐WB |
| | 0305 | | KGDFSK | | | KGD | | | | | |
| 936 | DRB1_ | 1009 | EFEYNYDPL | 3 | 1278 | YNYDPL | 0.793 | Sequence | 0.259511 | 4.72 | ⇐WB |
| | 0405 | | KGDFSK | | | KGD | | | | | |
| 936 | DRB1_ | 1009 | EFEYNYDPL | 3 | 1278 | YNYDPL | 0.88 | Sequence | 0.285332 | 4.96 | ⇐WB |
| | 0803 | | KGDFSK | | | KGD | | | | | |
| 936 | DRB1_ | 1009 | EFEYNYDPL | 5 | 1279 | YDPLKG | 0.953 | Sequence | 0.52626 | 2.68 | ⇐WB |
| | 1001 | | KGDFSK | | | DFS | | | | | |
| 936 | DRB1_ | 1009 | EFEYNYDPL | 3 | 1278 | YNYDPL | 0.987 | Sequence | 0.635007 | 1.33 | ⇐WB |
| | 1303 | | KGDFSK | | | KGD | | | | | |
| 937 | DRB1_ | 1010 | FEYNYDPLK | 4 | 1279 | YDPLKG | 1 | Sequence | 0.560414 | 2.12 | ⇐WB |
| | 0101 | | GDFSKI | | | DFS | | | | | |
| 937 | DRB1_ | 1010 | FEYNYDPLK | 4 | 1279 | YDPLKG | 0.987 | Sequence | 0.599202 | 1.92 | ⇐WB |
| | 1001 | | GDFSKI | | | DFS | | | | | |
| 937 | DRB1_ | 1010 | FEYNYDPLK | 2 | 1278 | YNYDPL | 0.967 | Sequence | 0.387503 | 3.67 | ⇐WB |
| | 1303 | | GDFSKI | | | KGD | | | | | |
| 938 | DRB1_ | 1011 | EYNYDPLKG | 3 | 1279 | YDPLKG | 1 | Sequence | 0.705123 | 1.33 | ⇐WB |
| | 0101 | | DFSKIG | | | DFS | | | | | |
| 938 | DRB1_ | 1011 | EYNYDPLKG | 3 | 1279 | YDPLKG | 1 | Sequence | 0.464316 | 3.45 | ⇐WB |
| | 0102 | | DFSKIG | | | DFS | | | | | |
| 938 | DRB1_ | 1011 | EYNYDPLKG | 3 | 1279 | YDPLKG | 0.98 | Sequence | 0.312996 | 3.58 | ⇐WB |
| | 0103 | | DFSKIG | | | DFS | | | | | |
| 938 | DRB1_ | 1011 | EYNYDPLKG | 3 | 1279 | YDPLKG | 1 | Sequence | 0.679572 | 1.26 | ⇐WB |
| | 1001 | | DFSKIG | | | DFS | | | | | |
| 939 | DRB1_ | 1012 | YNYDPLKG | 2 | 1279 | YDPLKG | 1 | Sequence | 0.323518 | 4.22 | ⇐WB |
| | 0101 | | DFSKIGT | | | DFS | | | | | |
| 939 | DRB1_ | 1012 | YNYDPLKG | 2 | 1279 | YDPLKG | 0.967 | Sequence | 0.410043 | 4.44 | ⇐WB |
| | 1001 | | DFSKIGT | | | DFS | | | | | |
| 953 | DRB1_ | 1013 | TTTKLVPYS | 4 | 1280 | LVPYSLD | 1 | Sequence | 0.448318 | 1.97 | ⇐WB |
| | 0402 | | LDSSYK | | | SS | | | | | |
| 954 | DRB1_ | 1014 | TTKLVPYSL | 3 | 1280 | LVPYSLD | 1 | Sequence | 0.566322 | 1.15 | ⇐WB |
| | 0402 | | DSSYKK | | | SS | | | | | |
| 954 | DRB1_ | 1014 | TTKLVPYSL | 3 | 1280 | LVPYSLD | 0.987 | Sequence | 0.213327 | 4.71 | ⇐WB |
| | 1501 | | DSSYKK | | | SS | | | | | |
| 955 | DRB1_ | 1015 | TKLVPYSLD | 5 | 1281 | YSLDSSY | 0.967 | Sequence | 0.252634 | 4.48 | ⇐WB |
| | 0301 | | SSYKKE | | | KK | | | | | |
| 955 | DRB1_ | 1015 | TKLVPYSLD | 5 | 1281 | YSLDSSY | 0.98 | Sequence | 0.225106 | 2.18 | ⇐WB |
| | 0305 | | SSYKKE | | | KK | | | | | |
| 955 | DRB1_ | 1015 | TKLVPYSLD | 2 | 1280 | LVPYSLD | 0.967 | Sequence | 0.379301 | 2.73 | ⇐WB |
| | 0402 | | SSYKKE | | | SS | | | | | |
| 956 | DRB1_ | 1016 | KLVPYSLDS | 4 | 1281 | YSLDSSY | 0.987 | Sequence | 0.281707 | 4.03 | ⇐WB |
| 0 | 301 | | SYKKEL | | | KK | | | | | |
| 956 | DRB1_ | 1016 | KLVPYSLDS | 4 | 1281 | YSLDSSY | 0.993 | Sequence | 0.250266 | 1.9 | ⇐WB |
| | 0305 | | SYKKEL | | | KK | | | | | |
| 957 | DRB1_ | 1017 | LVPYSLDSS | 3 | 1281 | YSLDSSY | 1 | Sequence | 0.302538 | 3.76 | ⇐WB |
| | 0301 | | YKKELY | | | KK | | | | | |
| 957 | DRB1_ | 1017 | LVPYSLDSS | 3 | 1281 | YSLDSSY | 0.98 | Sequence | 0.266742 | 1.74 | ⇐WB |
| | 0305 | | YKKELY | | | KK | | | | | |

### EXAMPLES

The following Examples are illustrative and are not intended to limit the scope of the claimed subject matter.

The technology disclosed and exemplified herein has profound implications for diagnosis and treatment of Lyme disease and post-treatment Lyme disease syndrome (PTLDS). T cell responses to B. burgdorferi can now be tracked during and after antibiotic therapy and the powerful T cell arm of the immune system can now be harnessed to hunt down and destroy any bacterial antigens that are sequestered in immune-privileged sites, such as dense fibrotic tissue, following antibiotic therapy. A similar approach is used to treat other hard-to-treat infectious diseases such as tuberculosis and is now being adopted for new types of cancer therapy.

### Example 1—Identification of Highly Immunogenic Epitope of B. burgdorferi

Using an immunopeptidomics approach we have recently identified and validated a highly immunogenic CD4+ T cell epitope derived from a protein involved in chemotaxis produced during B. burgdorferi infection (Peptide 1 (SEQ ID NO: 1)). FIG. 1. (MCP4442-462), hereafter Peptide 1 (SEQ ID NO: 1) is a methyl-accepting chemotaxis protein that is highly conserved amongst both sensu lato and sensu stricto strains of Lyme disease spirochetes (FIG. 3, FIG. 5). Peptide 1 is highly immunogenic in mice infected with B. burgdorferi (FIG. 4), validating Peptide 1 as target of CD4+ T cell responses to B. burgdorferi infection. Peptide 1 is one of a number of peptides derived from MCP4 predicted to bind to MHC class II alleles in mice and/or humans (FIG. 5). It is anticipated that immunization with Peptide 1 will lead to protective immunity in mice and humans. It is also anticipated that other epitopes derived from MCP4 are immunogenic in mice and humans infected with B. burgdorferi.

### Example 2—Test the Therapeutic Potential of Generating Immune Responses to Peptide 1 in Mice

Mice will be immunized with one or more of peptide 1 (SEQ ID NO: 1) and/or Peptide 2 (SEQ ID NO: 2) and tested for protection against B. burgdorferi infection. It is anticipated that immunized mice will exhibit fewer or no symptoms of infection compared to unimmunized controls mice. It is further anticipated that mice immunized with a vaccine composition comprising SEQ ID NO: 1 and/or SEQ ID NO: 2, or a polypeptide at least 90% identical to SEQ ID NO: 1 and/or a polypeptide at least 90% identical to SEQ ID NO: 2, will exhibit fewer or no symptoms of infection compared to mice immunized with prior art vaccines. It is further anticipated that mice immunized with a vaccine composition comprising SEQ ID NO: 1 and/or SEQ ID NO: 2, or a polypeptide at least 90% identical to SEQ ID NO: 1 and/or a polypeptide at least 90% identical to SEQ ID NO: 2, will exhibit an increase in CD4+ T-cell population as compared to unimmunized control mice, or mice immunized with prior art vaccines.

### Example 3—Determine Immune Responses to Target Antigen in Human Patients During Active Infection and in Patients with Post-Treatment Lyme Disease Syndrome (PTLDS)

Biobanks comprising Lyme disease patient samples will be evaluated to measure adaptive T and B cell responses to the target antigen (SEQ ID NO: 1 and/or SEQ ID NO: 2, or a polypeptide at least 90% identical to SEQ ID NO: 1 and/or a polypeptide at least 90% identical to SEQ ID NO: 2) during early infection, late infection, and post-antibiotics (PTLDS). It is anticipated that strong T cell response will be exhibited against SEQ ID NO: 1 and/or SEQ ID NO: 2 as compared to other antigens evaluated.

Lyme disease patients will be vaccinated with a composition comprising SEQ ID NO: 1 and/or SEQ ID NO: 2, or a polypeptide at least 90% identical to SEQ ID NO: 1 and/or a polypeptide at least 90% identical to SEQ ID NO: 2, and samples will be evaluated to measure adaptive T and B cell responses to the target antigen (SEQ ID NO: 1 and/or SEQ ID NO: 2, or a polypeptide at least 90% identical to SEQ ID NO: 1 and/or a polypeptide at least 90% identical to SEQ ID NO: 2) during early infection, late infection, and post-antibiotics (PTLDS). It is anticipated that strong T cell response will be exhibited against SEQ ID NO: 1 and/or SEQ ID NO: 2 as compared to unvaccinated controls, or as compared to other antigens evaluated.

### Example 4—Develop Diagnostic Reagents that can be Used to Measure the Evolution of CD4+ T Cell Responses to B. burgdorferi Before and After Antibiotic Therapy

A panel of tetramer reagents will be generated that can be used to fluorescently label individual CD4+ T cell responses to the target antigen in a wide range of genetic backgrounds. This set of tools will be used to generate a diagnostic platform to measure T cell responses to B. burgdorferi in patients both before and after antibiotic therapy. Exemplary diagnostic reagents include ELISpot assays, MCH tetramer assays, and T cell proliferation assays.

### Example 5—Identification of Highly Immunogenic Epitope of B. burgdorferi

Using an immunopeptidomics approach as described above and in FIG. 1 we have recently identified several peptides bound to MHC class II molecules expressed on murine macrophages cocultured with Borrelia burgdorferi (FIG. 6). The most abundant peptide of those listed in FIG. 6 is derived from BB0838 (LptD₉₂₃₋₉₃₄), hereinafter peptide 2 (SEQ ID NO: 2). LptD is an outer membrane protein (FIG. 7) involved in translocation of lipoproteins such as OspA and OspC from the periplasm to the outer membrane and is essential for cell growth. The LptD₉₂₃₋₉₃₄ epitope is located at the interface between the central β-barrel domain and the C-terminal β-barrel/plug domain (FIG. 7). Based on structure modeling, this epitope is localized at or near the cell surface, within the outer membrane-spanning β-barrel (FIG. 7). LptD is highly conserved amongst both sensu lato and sensu stricto strains of Lyme disease spirochetes, and the region surrounding the LptD₉₂₃₋₉₃₄ epitope includes numerous promiscuous epitopes, or peptides predicted to bind to a large number of human HLADRB1 alleles (FIG. 8). Peptide 2 is one of a number of peptides derived from LptD predicted to bind to MHC class II alleles in humans (FIG. 8 and Table 3). It is anticipated that immunization with Peptide 2 will lead to protective immunity in humans and mice. It also anticipated that other epitopes derived from LptD are immunogenic in humans and mice infected with B. burgdorferi. In some embodiments, and pursuant to the methods disclosed herein, T and B cell reactivity to LptD in humans with early and late Lyme disease, and in patients with PTLDS will be measured.

### Example 6—Identification of Bb-Derived MHC Class II Epitopes that are Presented by Antigen Presenting Cells (APCs) in Mouse Lymph Nodes During Infection, and by Human and Mouse APCs Stimulated with Bb In Vitro

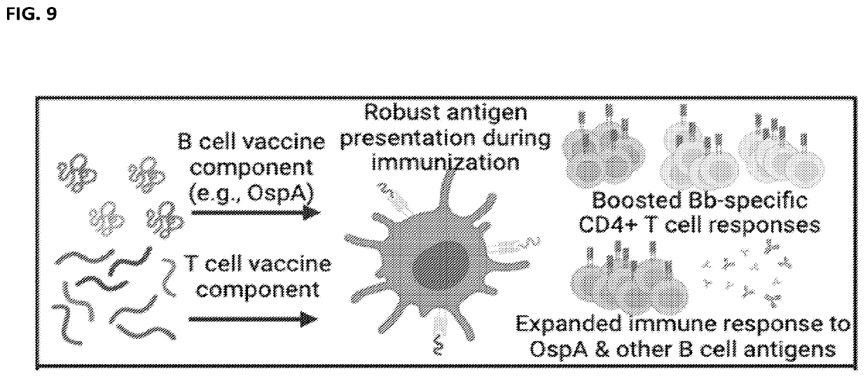

There is an urgent and unmet need for a safe and effective Lyme disease (LD) vaccine. Current efforts are underway to generate a next-generation LD vaccine for use in humans. The first-generation vaccine, LYMErix, targeted the Borrelia burgdorferi (Bb) outer surface protein A (OspA) immunogen, and is the basis for a current vaccine in clinical trials. While safe and somewhat effective, LYMErix and the current vaccine candidate targeting OspA share several weaknesses, including rapid down-regulation of the target antigen (OspA) during tick-to-host transmission, poor T-cell-dependent antibody responses to the immunogen, the need for multiple injections, limited cross-protection against different LD serotypes that express antigenically variable OspA types, and a loss of protective immunity ~6-12 months following initial immunization. Our discovery will address these weaknesses by using T-cell-directed LD vaccine component directed against conserved and constitutively expressed Bb MHC class epitopes we have recently identified. When used in conjunction with a B cell vaccine component using OspA or other outer surface proteins (Osps) as immunogens, this component will broaden and strengthen long-term protection against infection with antigenically diverse Bb genospecies (See FIG. 9).

Using an immunopeptidomics approach as described above and in FIG. 1 we have identified Bb-derived MHC class II epitopes that are presented by antigen presenting cells (APCs) in mouse lymph nodes during infection, and by human and mouse APCs stimulated with Bb in vitro (see Table 5 below). These MHC-II peptides will be synthesized, will be validated using mass spectrometry, and a subset are being tested for CD4+ T cell reactivity in infected and/or immunized mice. A vaccine component will be developed directed at conserved Bb MHC-II epitopes from proteins identified by LC-MS/MS (Table 5 below) to boost T cell, B cell, and antibody responses to humans or dogs immunized with Osp-based vaccines. It is believed that this will result in elevated levels of long-lived memory T cells, memory B cells, and plasma cells; higher-affinity and expanded antibody responses; and cross-protection against BQ sensu stricto (Bbss) and Db sensu lato (Bbsl) isolates with different OspA serotypes compared with Osp vaccination alone.

TABLE 5
MHC class II peptides identified by LC-MS/MS

| | | | | |
|---|---|---|---|---|
| | SEQ ID | | | |
| Gene ID | NO: | Name | Peptide | Source |

New peptides

| | | | | |
|---|---|---|---|---|
| BB_0283 | 7 | FlgE | FDVDSDRHLVNPANGMRIQGWMARDLEGEKVINTASDIED | Human |
| | | | | APCs |
| LRB56_ | 8 | Hypothetical | VFQFSVDIAKNFDNIEDGIKEYESKINKFEYRSGWEA | Human |
| 0950 | | protein | | APCs |
| BB_0389 | 9 | rpoB | AIDEEKKCIAQANAAFNSNGKYLEDLVSVRISGDYTTTSPTN | Human |
| | | | | APCs |
| BB_0604 | 10 | L-lactate | KNTISLSKGIIACSPYILIVTFIVLVSPLENKI | Human |
| | | permease | | APCs |
| BB_0578 | 11 | Mcp1 | KASISTLKVGFESLQRIL | Human |
| | | | | APCs |
| BB_0548 | 12 | polA | KNIKYEDVIQKNDNFANISL | Human |
| | | | | APCs |
| BB_0104 | 13 | HtrA | IVFAEEKDNTVRALQDSFRE | Human |
| | | | | APCs |
| BB_0679 | 14 | Ribose/ | ALNFLGPAIAVFISTLIF | Human |
| | | galactose | | APCs |
| | | ABC | | |
| | | transporter, | | |
| | | permease | | |
| | | protein | | |
| BB_0559 | 15 | crr | LEYLKEHSESVITPVVIANSDEVSSIEYSFGRLENDSEYILS | Human |
| | | | | APCs |
| BB_E09 | 16 | S2/P23 | YVTWAKSGDLRDIKDKNNN | Human |
| | | family | | APCs |
| | | protein | | |
| BB_P04 | 17 | Hypothetical | NGVIIKAGGGPPTAINIYAL | Human |
| | | protein | | APCs |
| BB_0612 | 18 | ClpX | ATTLTEAGYVGEDVE | Human |
| | | | | APCs |
| BB_RS06830 | 19 | ERF family | KIESDLILY | Human |
| | | protein | | APCs |
| BB_R28 | 20 | MlpD | TFKQVVQEALKGGIDG | Human |
| | | Lipoprotein | | APCs |
| BB_B23 | 21 | Putative | ESIGPIFNQL | Human |
| | | guanine/ | | APCs |
| | | xanthine | | |
| | | permease | | |
| BB_Q05 | 22 | lipoprotein | SEIIRNSGQI | Human |
| | | P35 | | APCs |

Peptides identified previously

| | | | | |
|---|---|---|---|---|
| BB0838 | 23 | LptD | NVFDFQFLFAMK | Mouse |
| | | | | APCs |
| BB_0559 | 24 | PtgA (crr) | HSESVITPVVIANSDE | Mouse |
| | | | | APCs |
| BB_0178 | 25 | MnmG | KPMILTRTSSYIGVLIDDLVTKGTKEPYRMFTSRAEHR | Mouse |
| | | | | APCs |
| BB_0680 | 26 | Mcp4 | KASLEVASSSQNLSSSALQQA | Mouse |
| | | | | Lymph |
| | | | | nodes |

### REFERENCES

Pietikäinen A, Backman I, Henningsson A J, Hytönen J. Clinical performance and analytical accuracy of a C6 peptide-based point-of-care lateral flow immunoassay in Lyme borreliosis serology. Diagn Microbiol Infect Dis. 2022 May; 103(1):115657. doi: 10.1016/j.diagmicrobio.2022.115657. Epub 2022 Feb. 3. PMID: 35228132.
Camire A C, Hatke A L, King V L, Millership J, Ritter D M, Sobell N, Weber A, Marconi R T. Comparative analysis of antibody responses to outer surface protein (Osp)A and OspC in dogs vaccinated with Lyme disease vaccines. Vet J. 2021 July; 273:105676. doi: 10.1016/j.tvjl.2021.105676. Epub 2021 Apr. 14. PMID: 34148599; PMCID: PMC8254658.

### Numbered Clauses

Clause 1. A polypeptide comprising or consisting of a fragment of BB0838 (LptD), or variant thereof.

Clause 2. A composition comprising the polypeptide of clause 1, optionally, wherein the polypeptide comprises SEQ ID NO: 2, or a polypeptide 90% identical to SEQ ID NO: 2, and an adjuvant.

Clause 3. The composition of clause 2, wherein the polypeptide is SEQ ID NO: 2.

Clause 4. The composition any of the previous clauses, comprising at least one additional polypeptide.

Clause 5. The composition of any of the previous clauses, wherein the at least one additional polypeptide comprises OspA or OspC.

Clause 6. The composition of any one of clauses 2-5, wherein the composition is in lyophilized form.

Clause 7. A method of treating Lyme disease, or a method for the preventative treatment of Lyme disease, the method comprising: administering to a subject the polypeptide of clause 1, or the composition of any of clauses 2-6.

Clause 8. The method of clause 7, wherein the subject is asymptomatic for Lyme disease, but may have come in contact with a blacklegged tick selected from Ixodes scapularis or Ixodes pacificus.

Clause 9. The method of clause 8, wherein the possible contact was within 3 months.

Clause 10. The method of clause 8, wherein the possible contact was within 2 months.

Clause 11. The method of clause 8, wherein the possible contact was within 1 month.

Clause 12. The method of clause 8, wherein the possible contact was within 2 weeks.

Clause 13. The methods of clause 8, wherein the possible contact was within 1 week.

Clause 14. The method of clause 8, wherein the possible contact was within 3 days.

Clause 15. The method of clause 8, wherein the possible contact was within 1 day.

Clause 16. The method of any one of clauses 7-15, wherein the subject has not previously been treated for Lyme disease.

Clause 17. The method of clause 7, wherein the subject is asymptomatic for Lyme disease and has previously been treated for Lyme disease.

Clause 18. The method of clause 17, wherein the subject previously tested positive for Lyme disease by standard two-tier (STT) testing.

Clause 19. The method of clause 18, wherein the subject has previously been treated for Lyme disease and exhibits one or more of the following symptoms: erythema migrans, facial palsy, and arthritis; and/or the subject tests positive for Lyme disease in standard two-tier test (STT).

Clause 20. The method of clauses 19, wherein the subject has not received treatment for Lyme disease for at least 1 year.

Clause 21. The method of clauses 19, wherein the subject has not received treatment for Lyme disease for at least 5 years.

Clause 22. The method of clauses 19, wherein the subject has not received treatment for Lyme disease for at least 10 years.

Clause 23. The method of clauses 19, wherein the subject has not received treatment for Lyme disease for at least 15 years.

Clause 24. The method of clauses 7-23, wherein the Lyme disease is caused by a Borrelia sp. bacteria.

Clause 25. The method of any one of clauses 7-23, wherein the Lyme disease is caused by Borrelia burgdorferi.

Clause 26. A method for testing a subject for the presence of Borrelia sp. bacteria, the method comprising: contacting a subject sample to the polypeptide of clause 1.

Clause 27. The method of clause 26, wherein the polypeptide comprises SEQ ID NO: 2.

Clause 28. The method of clause 27, wherein the polypeptide is SEQ ID NO: 2.

Clause 29. The method of clause 26, wherein the polypeptide is linked to a solid support.

Clause 30. The method of clause 26, wherein the subject sample comprises a blood or serum sample.

Clause 31. A kit comprising the polypeptide of clause 1 linked to a solid support.

Clause 32. A polypeptide comprising or consisting of a fragment of BB0680 (MCP4), or variant thereof.

Clause 33. A composition comprising the polypeptide of clause 32, optionally, wherein the polypeptide comprises SEQ ID NO: 1, or a polypeptide 90% identical to SEQ ID NO: 1, and an adjuvant.

Clause 34. The composition of clause 33, wherein the polypeptide is SEQ ID NO: 1.

Clause 35. The composition clause 33 or 34, comprising at least one additional polypeptide.

Clause 36. The composition of any one of clauses 33-35, wherein the at least one additional polypeptide comprises OspA or OspC.

Clause 37. The composition of any one of clauses 33-36, wherein the composition is in lyophilized form.

Clause 38. A method of treating Lyme disease, or a method for the preventative treatment of Lyme disease, the method comprising: administering to a subject the polypeptide of clause 32, or the composition of any of clauses 33-37.

Clause 39. The method of clause 38, wherein the subject is asymptomatic for Lyme disease, but may have come in contact with a blacklegged tick selected from Ixodes scapularis or Ixodes pacificus.

Clause 40. The method of clause 39, wherein the possible contact was within 3 months.

Clause 41. The method of clause 39, wherein the possible contact was within 2 months.

Clause 42. The method of clause 39, wherein the possible contact was within 1 month.

Clause 43. The method of clause 39, wherein the possible contact was within 2 weeks.

Clause 44. The methods of clause 39, wherein the possible contact was within 1 week.

Clause 45. The method of clause 39, wherein the possible contact was within 3 days.

Clause 46. The method of clause 39, wherein the possible contact was within 1 day.

Clause 47. The method of any one of clauses 38-46, wherein the subject has not previously been treated for Lyme disease.

Clause 48. The method of clause 38, wherein the subject is asymptomatic for Lyme disease and has previously been treated for Lyme disease.

Clause 49. The method of clause 48, wherein the subject previously tested positive for Lyme disease by standard two-tier (STT) testing.

Clause 50. The method of clause 49, wherein the subject has previously been treated for Lyme disease and exhibits one or more of the following symptoms: erythema migrans, facial palsy, and arthritis; and/or the subject tests positive for Lyme disease in standard two-tier test (STT).

Clause 51. The method of clauses 49, wherein the subject has not received treatment for Lyme disease for at least 1 year.

Clause 52. The method of clauses 49, wherein the subject has not received treatment for Lyme disease for at least 5 years.

Clause 53. The method of clauses 49, wherein the subject has not received treatment for Lyme disease for at least 10 years.

Clause 54. The method of clauses 49, wherein the subject has not received treatment for Lyme disease for at least 15 years.

Clause 55. The method of any one of clauses 38-54, wherein the Lyme disease is caused by a Borrelia sp. bacteria.

Clause 56. The method of any one of clauses 38-54, wherein the Lyme disease is caused by Borrelia burgdorferi.

Clause 57. A method for testing a subject for the presence of Borrelia sp. bacteria, the method comprising: contacting a subject sample to the polypeptide of clause 32.

Clause 58. The method of clause 57, wherein the polypeptide comprises SEQ ID NO: 1.

Clause 59. The method of clause 58, wherein the polypeptide is SEQ ID NO: 1.

Clause 60. The method of clause 57, wherein the polypeptide is linked to a solid support.

Clause 61. The method of clause 57, wherein the subject sample comprises a blood or serum sample.

Clause 62. A kit comprising the polypeptide of clause 32 linked to a solid support.

Clause 63. The composition of clause 4 or 5, wherein the at least one additional polypeptide comprises a polypeptide comprising or consisting of an amino acid sequence of SEQ ID NO: 1, or a polypeptide comprising or consisting of an amino acid sequence having 90% identity to SEQ ID NO: 1.

Clause 64. The composition of clause 4, 5, or 63, wherein the one additional polypeptide comprises: a polypeptide comprising or consisting of an amino acid sequence of SEQ ID NO: 3, or a polypeptide comprising or consisting of an amino acid sequence having 90% identity to SEQ ID NO: 3, a polypeptide comprising or consisting of an amino acid sequence of SEQ ID NO: 4, or a polypeptide comprising or consisting of an amino acid sequence having 90% identity to SEQ ID NO: 4, or a combination thereof.

Clause 65. The composition of clause 35 or 36, wherein the at least one additional polypeptide comprises a polypeptide comprising or consisting of the amino acid sequence of SEQ ID NO: 2, or a polypeptide comprising or consisting of an amino acid sequence having 90% identity to SEQ ID NO: 2.

Clause 66. The composition of clause 35, 36, or 65, wherein the one additional polypeptide comprises: a polypeptide comprising or consisting of an amino acid sequence of SEQ ID NO: 3, or a polypeptide comprising or consisting of an amino acid sequence having 90% identity to SEQ ID NO: 3, a polypeptide comprising or consisting of an amino acid sequence of SEQ ID NO: 4, or a polypeptide comprising or consisting of an amino acid sequence having 90% identity to SEQ ID NO: 4, or a combination thereof.

Clause 67. A composition comprising one or more of: a polypeptide at least 90% or 95% identical to SEQ ID NO: 1, a polypeptide at least 90% or 95% identical to SEQ ID NO: 2, a polypeptide at least 90% or 95% identical to SEQ ID NO: 3, or a polypeptide at least 90% or 95% identical to SEQ ID NO: 4.

Clause 68. The composition of clause 67, further comprising an adjuvant.

Clause 69. A method of treating Lyme disease, or a method for the preventative treatment of Lyme disease, the method comprising: administering to a subject the composition of clause 67.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A polypeptide comprising or consisting of a fragment of BB0838 (LptD), or variant thereof, the fragment comprising SEQ ID NO:2 or an amino acid sequence having at least 90% sequence identity to SEQ ID NO: 2.

2. A composition comprising the polypeptide of claim 1 and an adjuvant.

3. (canceled)

4. The composition of claim 2, comprising at least one additional polypeptide.

5. The composition of claim 4, wherein the at least one additional polypeptide comprises OspA or OspC.

6. The composition of claim 2, wherein the composition is in lyophilized form.

7. A method of treating Lyme disease, or a method for the preventative treatment of Lyme disease, the method comprising: administering to a subject the composition of claim 2.

8. The method of claim 7, wherein the subject is asymptomatic for Lyme disease, but may have come in contact with a blacklegged tick selected from Ixodes scapularis or Ixodes pacificus.

9. The method of claim 8, wherein the composition is administered within 3 months of the possible contact.

10.-15. (canceled)

16. The method of claim 7, wherein the subject has not previously been treated for Lyme disease.

17. The method of claim 7, wherein the subject is asymptomatic for Lyme disease and has previously been treated for Lyme disease.

18. The method of claim 17, wherein the subject previously tested positive for Lyme disease by standard two-tier (STT) testing.

19. The method of claim 18, wherein the subject has previously been treated for Lyme disease and exhibits one or more of the following symptoms: erythema migrans, facial palsy, and arthritis; and/or the subject tests positive for Lyme disease in standard two-tier test (STT).

20. The method of claim 19, wherein the subject has not received treatment for Lyme disease for at least 1 year.

21.-23. (canceled)

24. The method of claim 7, wherein the Lyme disease is caused by a Borrelia sp. bacteria.

25. The method of claim 7, wherein the Lyme disease is caused by Borrelia burgdorferi.

26. A method for testing a subject for the presence of Borrelia sp. bacteria, the method comprising: contacting a subject sample to the polypeptide of claim 1.

27. (canceled)

28. (canceled)

29. The method of claim 26, wherein the polypeptide is linked to a solid support and/or wherein the subject sample comprises a blood or serum sample.

30. (canceled)

31. A kit comprising the polypeptide of claim 1 linked to a solid support.

32.-63. (canceled)

64. The composition of claim 4, wherein the one additional polypeptide comprises: a polypeptide comprising or consisting of an amino acid sequence of SEQ ID NO: 3, or a polypeptide comprising or consisting of an amino acid sequence having 90% identity to SEQ ID NO: 3, a polypeptide comprising or consisting of an amino acid sequence of SEQ ID NO: 4, or a polypeptide comprising or consisting of an amino acid sequence having 90% identity to SEQ ID NO: 4, or a combination thereof.

65.-69. (canceled)
